(19) United States
(12) Patent Application Publication
(10) Pub. No.: US 2017/0226507 A1
(43) Pub. Date: Aug. 10, 2017

(54) COORDINATE CONTROL OF PATHOGENIC SIGNALING BY THE MIR-130/301 FAMILY IN PULMONARY HYPERTENSION AND FIBROPROLIFERATIVE DISEASES

(71) Applicant: The Brigham and Women's Hospital, Inc., Boston, MA (US)

(72) Inventors: Stephen Y. CHAN, Boston, MA (US); Thomas BERTERO, Boston, MA (US)

(73) Assignee: The Brigham and Women's Hospital, Inc., Boston, MA (US)

(21) Appl. No.: 15/308,777

(22) PCT Filed: May 5, 2015
(86) PCT No.: PCT/US15/29286

§ 371 Date: Nov. 3, 2016

Related U.S. Application Data
(60) Provisional application No. 61/988,550, filed on May 5, 2014.

(51) Int. Cl. C12N 15/113

(52) U.S. Cl. CPC C12N 15/113; C12N 2310/113; C12N 2310/141; C12N 2310/315; C12N 2310/322; C12N 2310/321; C12N 2310/3231

(57) ABSTRACT

The present invention relates to methods, kits and compositions to treat hypertension in a subject comprising inhibiting activity or expression of at least one microRNA.

### RELATED APPLICATIONS

This application claims benefit under 35 U.S.C. §119(e) of the U.S. Provisional Application No. 61/988,550, filed May 5, 2014, the content of which is incorporated herein by reference in its entirety.

### TECHNICAL FIELD

The present disclosure relates generally to methods to treat hypertension and fibrotic disease by using inhibiting one or more members of miR-130 and miR-301 families. Other aspects relates to compositions, methods and kits comprising inhibitors of miR-130/301 family, and methods, assays and arrays to assess a subject amenable to treatment with an inhibitor of miR-130/301 use in a method to to treat hypertension and fibrotic disease in a subject.

### BACKGROUND

Pulmonary hypertension (PH) is a complex vascular disease involving disparate molecular pathways spanning multiple cell types. MicroRNAs (miRNAs) may coordinately regulate these processes, but their integrative functions have been challenging to define with conventional approaches. As the number and complexity of known pathways known to be involved in PH has expanded, an understanding of global disease gene architecture has become a more pressing need.

### SUMMARY

Aspects disclosed herein are based, in part, on inventors' discovery that the microRNA-130/301 family acts as a unique, overarching pathogenic lynchpin, controlling multiple target genes in order to coordinate broad control over vascular tone, stiffness, and pulmonary hypertension (PH). The inventors' also discovered that inhibiting multiple members of the miR-130/301 family surprisingly and unexpectedly provides enhanced response in disease amelioration, as compared with inhibition of a single miRNA alone.

Accordingly, in one aspect, the disclosure provides a method of method of inhibiting, preventing or treating pulmonary hypertension (PH) or pulmonary arterial hypertension (PAH) or a symptom thereof in a subject. Generally the method comprises inhibiting activity of at least one member (e.g., one, two, three, four, five or more members) of microRNA-130/301 family in the subject. The disclosure also provides a method of inhibiting, preventing or treating a fibrotic or fibroproliferative disease or a symptom thereof in a subject in need thereof. Generally the method comprises inhibiting activity of at least one member (e.g., one, two, three, four, five or more members) of microRNA-130/301 family in the subject. The disclosure further provides a method of inhibiting or reducing extracellular matrix deposition or vascular/tissue stiffness in a subject in need thereof. Generally the method comprises inhibiting activity of at least one member (e.g., one, two, three, four, five or more members) of microRNA-130/301 family in the subject.

As noted-above, inhibiting multiple members of the miR-130/301 family surprisingly and unexpectedly provides enhanced response in disease amelioration, as compared with inhibition of a single miRNA alone. Thus, in some embodiments, the method comprises inhibiting the activity of at least two of, at least three of, at least four or at least five of miR-130a, miR-130b, miR-301a, miR-301b, and miR-454. Accordingly, in some embodiments, the method comprises inhibiting the activity of at least two members of miR-130/301 family, for example, method comprises inhibiting the activity of at least miR-130a and miR-130b; at least miR-130a and miR-301a; at least miR-130a and miR-301b; at least miR-130b and miR-301a; at least miR-130b and miR-301b; at least miR-301a and miR-301b; at least miR-130a and miR-454; at least miR-130b and miR-454; at least miR-301a and miR-454; or at least miR-301b and miR-454. In some embodiments, the method comprises inhibiting the activity of at least three members of miR-130/301 family, for example, method comprises inhibiting the activity of at least miR-130a, miR-130b and miR-301a; at least miR-130a, miR-130b and miR-301b; at least miR-130b, miR-301a and miR-301b; at least miR-130a, miR-130b and miR-454; at least miR-130a, miR-301a and miR-454; at least miR-130a, miR-301b and miR-454; at least miR-130b, miR-301a and miR-454; at least miR-130b, miR-301b and miR-454; or at least miR-301a, miR-301b and miR-454. In some embodiments, the method comprises inhibiting the activity of four members of the miR-130/301 family, for example, method comprises inhibiting the activity of miR-130a, miR-130b, miR-301a and miR-301b; miR-130a, miR-130b, miR-301a and miR-454; miR-130a, miR-130b, miR-301b and miR-454; miR-130a, miR-301a, miR-301b and miR-454; or miR-130b, miR-301a, miR-301b and miR-454.

In embodiments of the various aspects disclosed herein, the method comprises administering to the subject an effective amount of an agent that inhibits the activity of at least one member (e.g., one, two, three, four, five or more members) of microRNA-130/301 family. Without limitations the miR-130/301 family member can be selected from the group consisting of miR-130a, miR-130b, miR-301a, miR-301b, and any combinations thereof. In some embodiments, the miR-130/301 family member includes miR-454, which has the same seed sequence.

The agent for inhibiting the activity of miR-130/301 can be, for example, a small molecule, nucleic acid, nucleic acid analogue, peptide, protein, antibody, or variants and fragments thereof. In some embodiments, the nucleic acid agent can be DNA, RNA, nucleic acid analogue, peptide nucleic acid (PNA), pseudo-complementary PNA (pcPNA), locked nucleic acid (LNA) or analogue thereof. In some embodiments, the nucleic acid agent can be a small inhibitory RNA (RNAi), siRNA, microRNA, shRNA, miRNA and analogues and homologues and variants thereof effective in gene silencing.

In some embodiments, the agent is an oligonucleotide which comprises a nucleotide sequence which is substantially complementary to at least part of a miR-130/301 nucleotide sequence. For example, the oligonucleotide can comprise a nucleotide sequence which is substantially complementary to at least part of a pre-miR-130/301 nucleotide sequence, for example, pre-miR-130a, pre-miR-130b, pre-miR-301a and/or pre-miR-301b sequence. In some embodiments, the oligonucleotide can comprises a nucleotide sequence which is substantially complementary to at least part of a mature miR-130/301 sequence, for example, mature miR-130a, miR-130b, miR-301a and/or miR-301b sequence.

In some embodiments, the agent is an oligonucleotide which comprises a nucleotide sequence which is substantially complementary to seed sequence of miR-130/301. In some embodiments, the oligonucleotide comprises a nucleotide sequence which is substantially complementary to seed sequence of miR-130a, miR-130b, miR-301a and/or miR-301b sequence. In some embodiments, the oligonucleotide comprises a nucleotide sequence which is substantially complementary to 5′-AGUGCAA-3′ (SEQ ID NO: 1).

In some embodiments, the agent is an oligonucleotide comprising a nucleotide sequence complementary to the seed sequence, i.e., complementarity to the sequence 5′-AGUGCAA-3′ (SEQ ID NO: 1). Accordingly, in some embodiments, the agent is an oligonucleotide comprising the nucleotide sequence 5′-TTGCACT-3′ (SEQ ID NO: 2) or 5′-ATTGCACT-3′ (SEQ ID NO: 3).

In some embodiments, the oligonucleotide comprises nucleic acid modification. Exemplary nucleic acid modifications include, but are not limited to, nucleobase modifications, sugar modifications, inter-sugar linkage modifications, backbone modifications, and any combinations thereof. Nucleic acid modifications are described below in more detail.

In some embodiments, the agent is an antagomir, fully 2′-O-methoxyethyl (2′-MOE), 2′-F/MOE mixmer, fully LNA, LNA/DNA mixmer, a tiny LNA or a combination thereof. In some embodiments, the agent is a Tiny LNA oligonucleotide. As used herein, the term “tiny LNA” refers to a short, e.g., 6, 7, 8, 9, 10, 11 or 12-mer oligonucleotide that is comprised entirely of locked nucleic acid monomers. Tiny LNAs have been demonstrated to be effective to inhibit miRNA mediated gene suppression in vivo and are described in Obad et al., (Nature Genetics, 2010, 43(4): 371-380, content of which is incorporated herein by reference. In some embodiments, the agent is a shortmer. As used herein, the term “shortmer” refers to a short, e.g., 6, 7, 8, 9, 10, 11 or 12-mer oligonucleotide that is comprised entirely of 2′-MOE modified nucleic acid monomers.

In some embodiments, the oligonucleotide agent can be encoded by an expression vector.

In some embodiments, administration of the agent is prophylactic administration and in alternative embodiments, administration is therapeutic administration. In some embodiments, the methods and compositions as disclosed herein can be administered to a subject, where the subject is, for example, a mammal such as a human.

The method can further comprise selecting a subject for treatment for PH or PAH before onset of said administration, wherein the subject has elevated level of at least one member (e.g., one, two, three, four or five or more members) of miR-130/301 family relative to a control or reference level. Accordingly, in some embodiments, the method further comprises assaying a biological sample from the subject for elvated levels of one or more of (e.g., one, two, three, four, five or more of) miR130/301 family members relative to a reference or control level and selecting the subject who has elevated levels of at least one member (e.g., one, two, three, four, five or more members) of miR-130/301 family. For example, selecting the subject for treatment who has elevated level of at least one of miR-130a, miR-130b, miR-301a, and miR-301b relative to control or reference level.

In some embodiments, the method further comprises co-administering the agent with an additional therapeutic agent. In some embodiments, the additional thereapeutic agent is for treatment of hypertension, e.g., PH or PAH. In some other embodiments, the additional therapeutic agent is for treatment of a fibrotic or fibroproliferative disease. In still some other embodiments, the therapeutic agent is for inhibiting or reducing extracellular matrix deposition or vascular stiffness.

The disclosure also provides an isolated or synthetic oligonucleotide which can be used as an inhibitor of miR-130/301, i.e., an-anti-miR-130/301 agent. In some embodiments, the oligonucleotide comprises the nucleotide sequence 5′-TTGCACT-3′ (SEQ ID NO: 2) or 5′-ATTGCACT-3′ (SEQ ID NO: 3).

In some embodiments, the anti-miR-130/301 agent can be formulated as a pharmaceutical composition, i.e., a composition comprising the anti-miR-130/301 agent and a pharmaceutically acceptable carrier.

The disclosure further provides an assay to determine if a subject is at risk of pulmonary hypertension or PAH or a fibrotic or fibroproliferative disease. The assay comprising contacting a biological sample obtained from the subject with one or more probes to detect the levels of at least one (e.g., one, two, three, four or more) of microRNA-130a, microRNA-130b, microRNA-301a and microRNA-301b, wherein the level of at least one (e.g., one, two, three, four or more) of microRNA-130a, microRNA-130b, microRNA-301a or microRNA-301b above a predefined reference level identifies the subject predicted to be at risk of pulmonary hypertension or PAH or a fibrotic or fibroproliferative disease. In some embodiments, the assay comprises detecting the level of at leas one (e.g., one, two or three) of microRNA-130b, microRNA-301a and microRNA-301b. In some embodiments, the method comprises detecting the level of at least one of (e.g., one, two or three) of microRNA-130b, microRNA-301a and microRNA-301b.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1D show that in silico network-based analysis identifies miR-130/301 family as a master regulator of subordinate miRNA pathways and PH. (FIG. 1A) The expanded PH network is color-coded according to functional pathway. Encircled areas represent architectural clusters based on a spectral partition-based clustering algorithm. The miR-130/301 family was ranked the highest by a miRNA spanning score, reflecting the most robust systems-level control over the expanded PH network as a whole. Direct targets of the miR-130/301 family (28 enlarged nodes) span all 8 gene clusters and 13 functional pathways. (FIG. 1B) The miR-130/301 family members share the same seed sequence 5′-AGUGCAA-3′ (SEQ ID NO: 1). Sequences shown in FIG. 1B are miR-130a (CAGUGCAAUGUUAAAAGGGCAU (SEQ ID NO: 4)), miR-130b CAGUGCAAUGAUGAAAGGGCAU (SEQ ID NO: 5)), miR-301a (CAGUGCAAUAGUAUUGUCAAAGC (SEQ ID NO: 6)), and miR-301b (CAGUGCAAUGAUAUUGUCAAAGC (SEQ ID NO: 7)). (FIG. 1C) Direct targets of the miR-130/301 family, color-coded by functional pathway. (FIG. 1D) Predictions of downstream pathways connected to the miR-130/301 family and carrying the broadest influence on the expanded PH network, according to four scoring algorithms: (1) MiRNA Spanning Score: miRNA were ranked based on their network influence (see also Table 2), (2) Target Spanning Score: Targets of miR-130/301 were ranked based on their network influence and the robustness of their relationship to the miR-130/301 family (see also Table 3); (3) Gene Spanning Score: All network nodes were ranked based on their network influence (see also Table 4); (4) Shared miRNA Influence Score: miRNA were ranked based on the overlap of their PH-relevant target pool with the miR-130/301 family (see also Table 5).

FIGS. 2A-2D show that up-regulation of the miR-130/301 family by hypoxia is mediated by HIF-2α and POU5F1/OCT4. (FIG. 2A) As demonstrated by RT-qPCR, hypoxia (0.2% O₂ for 24 hours) up-regulated the miR-130/301 family in both human PAECs and PASMCs as compared with normoxia (21% O2 for 24 hours). (FIG. 2B) In PAECs transfected with siRNA control (si-NC) or siRNA specific for HIF-1α (si-HIF-1α), hypoxia up-regulated all miR-130/301 family members compared with normoxia. In contrast, in normoxia, miR-130/301 family members were down-regulated during HIF-2α knockdown (si-HIF-2α) compared with control. Moreover, during HIF-2αknockdown, family members were not induced by hypoxia. (FIG. 2C) In normoxic PAECs, lentiviral transduction with a constitutively expressed HIF-1αtransgene carrying a proline-to-alanine mutation (pHIF1) did not alter miRNA expression compared with transduction with empty vector (pEmpty) alone. In contrast, hypoxia up-regulated all miR-130/301 family members compared to normoxia in the presence of pEmpty alone. (FIG. 2D) During HIF-2α(si-HIF-2α) or POU5F1/OCT4 (si-OCT4) knockdown in PAECs, miR-130/301 family members were not induced by hypoxia, in contrast to cells transfected with siRNA control (si-NC). In each panel, for each miRNA, mean expression in normoxic control groups (21% O₂ in FIG. 2A; si-NC 21% O₂ in FIG. 2B, FIG. 2D; pEmpty 21% O₂ in (FIG. 2C) was assigned a fold change of 1, to which relevant samples were compared. Data are presented as mean±S.D. (*P<0.05 ** P<0.01).

FIGS. 3A-3F show that The miR-130/301 family is induced in multiple models of PH. (FIG. 3A) By RT-qPCR, miR-130/301 expression was increased in homogenized lung in mice suffering from PH induced by treatment with the VEGF receptor antagonist SU5416 in combination with chronic hypoxia (10% O₂ for 3 weeks; n=12) as compared with normoxia (21% O₂; n=12). (FIG. 3B) miR-130/301 progressively increased in homogenized lung of rats suffering from experimental PH induced by monocrotaline administration (n=8 rats/treatment group; wk=week(s)). (FIG. 3C) miR-130/301 was increased in homogenized lung of juvenile sheep suffering from shunt-induced PAH (Shunt; n=8) as compared with sham control groups (n=8). For each experiment, mean expression in control groups was assigned a fold change of 1, to which relevant samples were compared. In situ hybridization of serially sectioned formalin-fixed paraffin-embedded mouse lung (FIG. 3D) and quantification of <100 μm pulmonary vessels (5 vessels per mouse) (FIG. 3E) revealed that miR-130a was up-regulated in small pulmonary vessels (<100 μm) exposed to hypoxia with or without SU5416 (10% O₂) as compared with normoxia (21% O₂) with or without SU5416. (FIG. 3F) Intensity of miR-130a stain in situ positively correlated with pulmonary vessel thickness. Statistically significant differences are indicated (*P<0.05; ** P<0.01). In physiologic experiments (FIGS. 3A-3C), data are expressed as mean±SEM, while in in situ quantitations (FIGS. 3E-3F), data are expressed as mean±SD. Tissue scale bar 50 μm.

FIGS. 4A-4C show that expression of the miR-130/301 family is up-regulated in diverse forms of human PAH. In situ hybridization of human lung (FIG. 4A) and quantification of <200 μm pulmonary vessels (10 vessels per patient) (FIG. 4B) revealed increased miR-130a expression in diseased pulmonary vessels of 6 patients suffering from PAH stemming from a variety of causes as well as in vessels from 13 patients with scleroderma-induced PAH as compared with 4 healthy controls (no PH). Red arrow denotes intimal staining; black arrow denotes medial staining. (FIG. 4C) Increasingly higher plasma levels of miR-130/301 family members were observed in patients with increasing hemodynamic severity of PH. Here, 5 non-PH controls (mean pulmonary arterial pressure or mPAP<25 mmHg) were compared with two groups of PH patients stratified by hemodynamic severity—7 patients with mPAP between 25 to 45 mmHg and 7 patients with mPAP>45 mmHg. Statistically significant differences are indicated (*P<0.05; **P<0.01). In physiologic experiments (FIGS. 4A-4B), data are expressed as mean±SEM, while in in situ quantitations (panel B), data are expressed as mean±SD. Tissue scale bar 50 μm.

FIGS. 5A-5F show that The miR-130/301 family represses PPARγ in order to control proliferation in PAECs via the apelin/miR-424/503/FGF2 regulatory axis. In normoxia (21% O₂) or hypoxia (0.2% O₂, 24 hours), either forced expression of miR-130a mimic versus control (NC) or inhibition of miR-130a (anti-miR-130a) versus inhibition of the entire miR-130/301 family (tiny-LNA-130) versus control (NC) was performed in cultured PAECs (FIGS. 5A-5D). Immunoblotting (FIG. 5A), gel densitometry (FIG. 5B), and RT-qPCR (FIG. 5C and FIG. 5D) revealed that forced miR-130a expression decreased PPARγ, a direct target of miR-130/301 family (FIG. 13); decreased apelin (FIGS. 5A-5B), miR-424 (FIG. 5C), and miR-503 (FIG. 5D); and increased FGF2 (FIGS. 5A-5B). Importantly, inhibition of the entire miR-130/301 family reversed these downstream gene/subordinate miRNA alterations to a greater extent than inhibition of miR-130a alone (FIGS. 5A-5D), thus demonstrating the importance of the coordinated actions of this miRNA family. (FIG. 5E) As assessed by BrdU incorporation in exponentially growing PAECs, proliferation was augmented during forced expression of miR-130a mimics but decreased during expression of miR-424 or miR-503. Consistent with the direct dependence of miR-130a on miR-424 and miR-503 in PAECs, miR-130a-induced proliferation was reversed when miR-424, miR-503, or both together were simultaneously expressed. (FIG. 5F) In PAECs, we have validated that the miR-130/301-PPARγregulatory axis controls cellular proliferation by repressing apelin and miR-424/503 leading to an increase of FGF2. In (FIGS. 5C-5D), for each miRNA, mean expression in control groups (miR-NC or Anti-miR-NC) was assigned a fold change of 1, to which relevant samples were compared. In all panels, data are expressed as mean±SD (*P<0.05; ** P<0.01).

FIGS. 6A-6E show that The miR-130/301 family represses PPARγ in order to control proliferation in PASMCs via the STAT3/miR-204 regulatory axis. In normoxia (21% O₂) or hypoxia (0.2% O₂, 24 hours), either forced expression of miR-130a mimic versus control (NC) or inhibition of miR-130a (anti-miR-130a) versus inhibition of the entire miR-130/301 family (tiny-LNA-130) versus control (NC) was performed in cultured PASMCs (FIGS. 6A-6D). Immunoblotting (FIG. 6A), gel densitometry (FIG. 6B) and RT-qPCR (FIG. 6C) demonstrated that forced miR-130a expression decreased PPARγ, accompanied by an increase in STAT3 and activated phosphorylated STAT3 (P-STAT3; (FIGS. 6A-6B), as well as decreased miR-204 (FIG. 6C). Importantly, as in PAECs, inhibition of the entire miR-130/301 family in PASMCs reversed such downstream gene/subordinate miRNA alterations to a greater extent than inhibition of miR-130a alone (FIGS. 6A-6D), thus demonstrating the importance of the coordinated actions of this miRNA family. (FIG. 6D) As assessed by BrdU incorporation in exponentially growing PASMCs, proliferation was augmented during forced expression of miR-130a mimics but decreased during expression of miR-204. MicroRNA-130a induced proliferation was reversed when miR-204 was simultaneously expressed, thus confirming the direct dependence of miR-130a on miR-204 in this cell type. (FIG. 6E) In PASMCs, we have validated that the miR-130/301-PPARγ regulatory axis controls proliferation by repressing STAT3 expression and activity and subordinate miR-204 expression. In (FIG. 6C), for each miRNA, mean expression in control groups (miR-NC or Anti-miR-NC) was assigned a fold change of 1, to which relevant samples were compared. In all panels, data are expressed as mean±SD (*P<0.05; ** P<0.01).

FIGS. 7A-7D show that Forced expression of miR-130a induces PH in a PPARγ-dependent manner in vivo. (FIG. 7A) Serial intrapharyngeal delivery of miR-130a mimic oligonucleotide (miR-130a) increased miR-130a in whole lung in mice as compared with control (miR-NC), either with or without daily oral dosing of rosiglitazone (Rosi). See FIG. 27 for protocol design. (FIG. 7B) miR-130a increased right ventricular systolic pressure (RVSP) compared with control (miR-NC), an effect reversed by rosiglitazone. See FIG. 30A for quantification of right ventricle/(left ventricle+septum) (RV/LV+S) mass ratio. (FIG. 7C) By RT-qPCR of whole lung, miR-130a decreased miR-204, miR-322 (the mouse homolog of human miR-424) and miR-503 expression, but this effect was reversed by rosiglitazone. (FIG. 7D) Immunohistochemical (IHC) staining of <100 μm diameter pulmonary vessels was performed to quantify PPARγ, PCNA, phosphorylated STAT3 (P-STAT3), α-smooth muscle actin (α-SMA). Ten vessels were quantified for each mouse (bar graphs). Consistent with data from cultured cells (FIGS. 5-6), miR-130a decreased PPARγ expression (top row); increased PCNA-positive (second row) and P-STAT3-positive (third row) cells (quantified over 10 vessels); and increased medial thickness (bottom row) consistent with exaggerated vascular remodeling. See FIG. 30B for arteriolar density, FIG. 30C for arteriolar muscularization and FIGS. 30D-30E for gene expression by immunoblotting. In (FIGS. 7A and 7C), for each miRNA, mean expression in control groups (miR-NC) was assigned a fold change of 1, to which relevant samples were compared. In experiments involving animal tissue (FIGS. 7A-7C), data are expressed as mean±SEM, while IHC quantification (FIG. 7D) is expressed as mean±SD (*P<0.05 ** P<0.01). Scale bar 50 μm.

FIGS. 8A-8D show that The miR-130/301 family is necessary to induce PH in a hypoxic mouse model of disease. A protocol was performed to determine whether a “shortmer” inhibitor of miR-130/301 (short-130) reverses PH (hypoxia+SU5416) in mice. See FIG. 32. (FIG. 8A) While miR-130/301 progressively increased with exposure time to hypoxia+SU5416, short-130 maintained baseline miRNA expression (normoxia+SU5416). (FIG. 8B) RVSP progressively increased as mice were exposed to two weeks (Ctrl 2 weeks) and four weeks of hypoxia+SU5416 (PBS). Increased RVSP was abrogated by short-130 but not short-NC. See FIG. 35A for quantification of (RV/LV+S) mass ratio. (FIG. 8C) During hypoxia+SU5416, short-130 rescued miR-204, miR-322 (mouse homolog of human miR-424), and miR-503 expression in whole lung. (FIG. 8D) IHC staining of <100 μm diameter pulmonary vessels was performed to quantify PPARγ, PCNA, phosphorylated STAT3 (P-STAT3), α-smooth muscle actin (α-SMA). Ten vessels were quantified for each mouse (bar graphs). Inhibition of the miR-130/301 family after PH development preserved PPARγ expression (top row); decreased PCNA-positive (second row) and P-STAT3-positive (third row) cells (quantified over 10 vessels); and decreased medial thickness (bottom row) consistent with blunted vascular remodeling. See FIG. 35B for arteriolar density, FIG. 35C for arteriolar muscularization and FIGS. 35D-35E for immunoblotting. In (FIGS. 8A, 8C), for each miRNA, normoxic levels were assigned a fold change of 1, to which relevant samples were compared. Data involving animal tissue (FIGS. 8A-8C) are expressed as mean±SEM, while IHC quantification (FIG. 8D) is expressed as mean±SD (*P<0.05 ** P<0.01). Scale bar 50 μm.

FIG. 9 is a model of the actions of the miR-130/301 family in PH. Network-based bioinformatics coupled with experimental validation identifies the broad hierarchical control over proliferation exerted by the miR-130/301 family via integration of numerous subordinate miRNA pathways in multiple pulmonary vascular cell types.

FIGS. 10A-10D show up-regulation of the miR-130/301 family by inflammatory cytokines IL-1β and IL-6 as well as by siRNA knockdown of BMPR2 and CAV1. As demonstrated by RT-qPCR, exposure of PAECs or PASMCs with long/mL IL-1β (FIG. 10A) or 100 ng/mL IL-6 (FIG. 10B) for 24 hours (black bars) up-regulated miR-301a compared with vehicle-treated (gray bars) cells. Additionally, miR-301b was induced by IL-1β (n=3) in PASMCs (FIG. 10A) and in PAECs treated with IL-6 (FIG. 10B). Mean expression of miRNAs in vehicle control groups were assigned a fold change of 1, to which relevant samples were compared. (FIG. 10C) As demonstrated by RT-qPCR, siRNA knockdown of BMPR2 or CAV1 in PAECs (left graph) and PASMCs (right graph) up-regulated miR-130/301 compared with scrambled control (si-NC). However, siRNA knockdown of other factors genetically associated with PAH such as ACVR1L, ENG, KCNK3, and SMAD9 had negligible effects on miR-130/301 expression. Mean expression of miRNAs in control groups (si-NC) were assigned a fold change of 1, to which relevant samples were compared. (FIG. 10D) As demonstrated by RT-qPCR, effective siRNA knockdown was achieved in PAECs (left graph) and PASMCs (right graph) of factors genetically associated with PAH. For each gene transcript, mean expression in control groups (si-NC, light gray) were assigned a fold change of 1, to which relevant samples (transfected with a siRNA specific to that gene, black) were compared. Data are presented as mean±S.D. (*P<0.05, ** P<0.01).

FIGS. 11A-11E show that Up-regulation of the miR-130/301 family by HIF-2α is dependent upon POU5F1/OCT4. RT-qPCR expression was analyzed for EPAS1/HIF-2α and POU5F1/OCT4 in PAECs (FIGS. 11A-11B) and PASMCs (FIGS. 11C-11D) after transfection with siRNAs specific for OCT4 (si-OCT4), HIF-2α (si-HIF-2α) or siRNA control (si-NC) during 24 hour hypoxia (0.2% O₂) compared with normoxia (21% O₂). In both cell types transfected with si-NC, hypoxia up-regulated EPAS1/HIF-2α and POU5F1/OCT4 compared with normoxia. Knockdown of HIF-2α (si-HIF-2α) inhibited the induction of POU5F1/OCT4 by hypoxia (FIGS. 11B and 11D), thus confirming that HIF-2α up-regulates POU5F1/OCT4. However, knockdown of POU5F1/OCT4 (si-OCT4) did not prevent induction of HIF-2α by hypoxia. (FIG. 11E) During HIF-2α (si-HIF-2α) or POU5F1/OCT4 (si-OCT4) knockdown in PASMCs, miR-130/301 family members were not induced by hypoxia, in contrast to cells transfected with siRNA control (si-NC). In (FIG. 11A-11D), mean levels in control groups exposed to normoxia and siRNA control (21% O₂, si-NC) were assigned a fold change of 1, to which relevant samples were compared. In (FIG. 11E), control groups (21% O₂, si-NC) were separately assigned a fold change of 1 for each miRNA, to which relevant samples were compared. Data are presented as mean±SD (*P<0.05 ** P<0.01).

FIGS. 12A-12D show that expression of the miR-130/301 family is up-regulated in lung tissue of various mouse models of PH. (FIG. 12A) As demonstrated by RT-qPCR, expression of the miR-130/301 family members in homogenized lung was up-regulated in VHL−/− mice (VHL−/−; n=8) as compared with control mice (WT; n=10). (FIG. 12B) Up-regulation of miR-130b and miR-301a/b was observed in IL-6 transgenic mice in normoxia (21% O₂; n=8, light gray) as compared with wildtype littermates (21% O₂; n=8, white). For wildtype littermates, all miR-130/301 family members were up-regulated by hypoxia (10% O₂; n=8, dark gray) as compared with normoxia (21% O₂; n=8, white). Such differences in hypoxic versus normoxic miRNA expression were also observed in IL-6 transgenic mice. Notably, IL-6 transgenic mice exposed to hypoxia (10% O₂; n=8, black) displayed substantially greater expression of all miR-130/301 family members as compared with hypoxic wildtype littermates (10% O₂; n=8, dark gray). (FIG. 12C) miR-130/301 family members were up-regulated in homogenized lung of transgenic mice expressing a dominant negative BMPR2 (BMPR2X Tg; n=8) as compared with control mice (WT; n=8). In (FIGS. 12A-12C), mean miRNA levels in the WT normoxic groups were assigned a fold change of 1, to which all samples were compared. (FIG. 12D) miR-130/301 family members were up-regulated in homogenized lung of mice suffering from S. mansoni-induced PAH (n=4) as compared with non-infected control mice (Control; n=5). Mean miRNA expression in WT control groups was assigned a fold change of 1, to which all samples were compared. For each experiment PH was confirmed by assessment of RVSP. Data are presented as mean±SEM (*P<0.05, ** P<0.01).

FIGS. 13A-13B show that PPARγ is a direct target of miR-130/301 family. (FIG. 13A) Sequence alignment between miR-130a and the 3′UTR of PPARγ highlights a highly conserved putative binding site 5′-UUGCACUA-3′ (SEQ ID NO: 8). Sequences shown are Has (CCCUUCUUCCAGUUGCACUAUUCUG (SEQ ID NO: 9)), Mmu (UCCUUCUAUUGAUUGCACUAUUAUUUUG (SEQ ID NO: 10)), Rno (UCCUUCUAUCGAUUGCACUAUUAUUUUG (SEQ ID NO: 11)), Dno (UCCUCCUCCCAGUUGCACUAUUAUUUUG (SEQ ID NO: 12)), Xtr (ACUCGCCCCCAUUUGCACUAUUUCUAUA (SEQ ID NO: 13)), and hsa-mir-130a (3′-UACGGGAAAAUUGUAACGUGAC-5′ (SEQ ID NO. 14)). (FIG. 13B) A luciferase reporter assay confirmed the recognition of the binding site in the PPARγ transcript by miR-130a. HEK293T cells were transfected with a luciferase reporter construct carrying a 3′ UTR with either the predicted binding site for the miR-130/301 family encoded by the human PPARγ transcript (WT) or scrambled mutant (mut). In combination, cells were transfected with oligonucleotide mimics of miR-130a or control (miR-NC). miR-130a reduced Renilla luciferase activity as compared with miR-NC in the setting of the WT binding site, but induced no significant change in the setting of mutant binding site. Luciferase levels in cells transfected with miR-NC were normalized to 1, to which other conditions were compared. Data are presented as mean±SD (*P<0.05 ** P<0.01).

FIGS. 14A-14C show the comparison of the specificity of miR-130/301 oligonucleotide inhibitors. (FIG. 14A) Overview of the miRNA silencing approach using seed-targeting tiny locked nucleic acid (LNA) molecules. Tiny LNAs were designed as fully LNA-modified oligonucleotides complementary to the seed region of the miR-130/301 family. The high binding affinity of tiny LNA enables functional inhibition of co-expressed members of miRNA seed families, which leads to de-repression of target mRNAs. Sequences shown in FIG. 14A are miR-130a (SEQ ID NO: 4), miR-130b (SEQ ID NO: 5), miR-301a (SEQ ID NO: 6), miR-301b (SEQ ID NO: 7) and tiny-LNA-130 (ATTGCACT SEQ ID NO: 3). As show in FIG. 14A, sequences for SEQ ID NOs: 4-7 are written in the 3′->5′ direction (left to right) and SEQ ID NO: 3 is written in the 5′->3′ direction (left to right). In the setting of exposure to hypoxia (0.2% O₂; 24 h) or normoxia (21% O₂; 24 h), expression levels of miR-130/301 family members in PAECs (FIG. 14B) and PASMCs (FIG. 14C) were quantified after transfection with anti-miR-130a versus control (anti-miR-NC) (left panels) or tiny-LNA-130 versus control (tiny-LNA-NC) (right panels). Corresponding with our previous results, in both PASMCs and PAECs, RT-qPCR revealed an up-regulation of the miR-130/301 family in control treated cells in hypoxia (anti-miR-NC 0.2% O₂ or tiny-NC 0.2% O₂) compared to control cells in normoxia (anti-miR-NC 21% O₂ or tiny-NC 21% O₂). In cells treated with anti-miR-130a, miR-130a was specifically down-regulated in both hypoxia and normoxia compared with control cells transfected with anti-miR-NC. Notably, baseline expression and dynamic alterations of other miR-130/301 family members were not affected by anti-miR-130a. In contrast, in cells treated with tiny-LNA-130, all miR-130/301 family members were down-regulated in normoxia and hypoxia compared with cells treated with tiny-LNA-NC. In all panels, mean levels in control groups (Anti-miR-NC 21% O₂ or tiny-LNA-NC 21% O₂) were assigned a fold change of 1, to which relevant samples were compared. Data are presented as mean±SD (*P<0.05, ** P<0.01).

FIGS. 15A-15D shows that the miR-130/301 family promotes proliferation in PAECs and PASMCs. (FIG. 15A) Exponentially growing cells were transfected with miR-130a, anti-miR-130a, tiny-LNA-130 or matched controls and counted each day during 3 days. After 3 days, miR-130a increased significantly the number of human PAECs (left panel) and PASMCs (right panel) compared with control (miR-NC). Conversely, inhibition of miR-130a alone (anti-miR-130a) or all the family (tiny-LNA-130) decreased the number of cells compared to the respective control cells (anti-miR-NC and tiny-LNA-NC, respectively. Moreover, in PAECs and PASMCs, inhibition of the miR-130/301 family (tiny-LNA-130) led to a greater decrease in the number of cells compared with anti-miR-130a. (FIG. 15B) Exponentially growing human PAECs (left panel) and PASMCs (right panel) were transfected with miR-130a or miR-NC and pulsed with BrdU for 1 h. In both cell types, miR-130a increased BrdU incorporation and thus proliferation compared with miR-NC. (FIG. 15C) Exponentially growing human PAECs (left panel) and PASMCs (right panel) were transfected with anti-miR-130a or tiny-LNA-130 or relevant matched controls followed by a BrdU pulse for 1 hour. In both cell types, inhibition of miR-130a alone (anti-miR-130a) decreased BrdU incorporation and thus proliferation. Inhibition of the miR-130/301 family (tiny-LNA-130) led to a greater decrease in proliferation compared with anti-miR-130a, indicating functional redundancy in miR-130/301 family members. (FIG. 15D) Expression of the proliferation marker PCNA was quantified in PAECs (left panel) and PASMCs (right panel) 48 hours after transfection with miR-130a, anti-miR-130a, tiny-LNA-130 as compared with relevant controls. Correlating with results in (FIGS. 15A, 15B, 15C), immunoblotting and quantification revealed a modest increase in PCNA expression with miR-130a, a decrease with anti-miR-130a and a more substantial decrease with tiny-LNA-130. In all bar graphs (FIGS. 15A-15D), data are expressed as mean±SD (*P<0.05, ** P<0.01).

FIGS. 16A-16B show that siRNA knockdown of PPARγ induces proliferation in PAECs and PASMCs. (FIG. 16A) Exponentially growing cells were transfected with siRNA against PPARγ (si-PPARγ) or control (si-NC) and counted each day for 3 days. Knockdown of PPARγ increased cell number of PAECs (left panel) and PASMCs (right panel) compared with si-NC. (FIG. 16B) Exponentially growing PAECs (left panel) and PASMCs (right panel) were transfected with si-PPARγ or si-NC and pulsed with BrdU for 1 h. Knockdown of PPARγ increased BrdU incorporation and thus proliferation as compared with si-NC. In all panels, data are expressed as mean±SD (*P<0.05, ** P<0.01).

FIGS. 17A-17B show that Forced expression of PPARγ reverses miR-130a-dependent increases of proliferation. Forced expression of PPARγ was achieved in PAECs (left panels) and PASMCs (right panels) by lentiviral transduction with a constitutively expressed PPARγtransgene (pPPARγ) that does not encode for a 3′ untranslated region and thus missing the endogenous miR-130/301 binding site. After stable transduction of pPPARγ versus control GFP transgene alone (pGFP), exponentially growing cells were transfected with miR-130a or miR-NC followed by cell counting over three days (FIG. 17A) or BrdU pulse-labeling experiments (FIG. 17B) as described in FIGS. 15-16. In both cell types transfected with miR-NC, forced expression of PPARγ reduced the level of BrdU incorporation significantly (FIG. 17B), leading to a trend toward decreased cell number (FIG. 17A). Importantly, in both cell types transfected with miR-130a, the miR-130a-induced increases in cell number and BrdU incorporation (miR-130a+pGFP) were significantly reversed (miR-130a+pPPARγ) to baseline levels (miR-NC+pGFP), thus proving the critical importance of PPARγ repression in the proliferative actions of miR-130a. In all panels, data are expressed as mean±SD (*P<0.05, ** P<0.01).

FIGS. 18A-18B shows that the miR-130/301 family promotes apoptotic signaling in PAECs, but not PASMCs, during serum starvation. Exponentially growing PAECs (FIG. 18A) or PASMCs (FIG. 18B) were transfected with miR-130a, tiny-LNA-130 or matched controls. After two days, cells were serum-starved and assessed for the enzymatic activity of caspases 3/7 as a functional marker of apoptosis. Caspase activity was measured after 24 hours of serum deprivation. No significant modulation of caspase 3/7 activity was observed in PAECs or PASMCs grown in serum-replete conditions and transfected with miR-130a or tiny-LNA-130 as compared with control oligonucleotides (miR-NC and tiny-NC, respectively). However, induction of caspase 3/7 activity was observed in PAECs and PASMCs after 24 h of serum deprivation as compared with controls cells (24 hour starvation). In serum-starved PAECs, overexpression of miR-130a further increased caspase 3/7 activity. Conversely, inhibition of miR-130/301 family by tiny-LNA-130 partially protected cells from caspase 3/7 induction. In contrast, in serum-starved PASMCs, no significant modulation of caspase activity was observed with miR-130/301 manipulation. In all panels, mean levels in control group (miR-NC in serum-replete conditions) were assigned a fold change of 1, to which relevant samples were compared In all panels, data are expressed as mean±SD (*P<0.05, ** P<0.01).

FIGS. 19A-19D show that siRNA knockdown of PPARγ mimics the actions of the miR-130/301 family by down-regulating apelin, miR-424, and 503, and up-regulating FGF2 in PAECs. (FIGS. 19A-19C) Transcript levels of PPARγ (PPARG, left panel-FIG. 19A), apelin (APLN, right panel-FIG. 19A), and FGF2 (FIG. 19C), as well as expression of miR-424 (left panel-FIG. 19B) and miR-503 (right panel-FIG. 19B) were quantified in normoxic (21% O₂) PAECs 48 h after transfection with siRNA against PPARγ (si-PPARG) or siRNA control (si-NC). RT-qPCR confirmed PPARγ knockdown after treatment with si-PPARγ compared with control (si-NC, left panel-FIG. 19A). Under these same conditions, such PPARγ knockdown led to decreased APLN expression (right panel-A) as well as decreased miR-424 (left panel-FIG. 19B) and miR-503 (right panel-FIG. 19B) expression. (FIG. 19C) Conversely, PPARγ knockdown increased FGF2 transcript levels. (FIG. 19D) In normoxic PAECs treated with siRNA against PPARγ (PPARγ) or siRNA control (NC), immunoblotting revealed decreased protein levels of PPARγ and apelin but increased FGF2. Actin was used as a loading control. In (FIGS. 19A-19C), mean levels in control groups exposed to siRNA control (si-NC) were assigned a fold change of 1, to which relevant samples were compared. In all panels, data are expressed as mean±SD (*P<0.05, ** P<0.01).

FIGS. 20A-20H show that downstream targets dependent on PPARγ are modulated by miR-130/301 family in both PAECs and PASMCs. (FIGS. 20A-20D) Transcript levels of PPARγ-dependent targets CCND1, CDKN1A, CDKN2A, and CDKN1B were quantified in PAECs 48 h after transfection with (FIG. 20A) miR-130a or tiny-LNA-130 versus appropriated controls (miR-NC and tiny-LNA-NC respectively) or (FIG. 20B) with siRNA recognizing PPARγ (si-PPARγ) versus siRNA control (si-NC). Consistent with the known regulatory actions of PPARγ on these genes, PPARγ knockdown or miR-130a overexpression decreased CDKN1A, CDKN1B, and CDKN2A expression while increased CCND1 expression as compared with si-NC control. Conversely, inhibition of miR-130/301 family (tiny-LNA-130) increased CDKN1A, CDKN1B, and CDKN2A expression while decreased CCND1 expression as compared with control (tiny-LNA-NC). (FIGS. 20C-20D) In PAECs, forced expression of PPARγ was achieved by lentiviral transduction with a constitutively expressed PPARγ transgene (pPPARγ) that does not encode for a 3′ untranslated region and thus missing the endogenous miR-130/301 binding site. Alternatively, pharmacologic activation of PPARγ activity was achieved by 24 h treatment with rosiglitazone (10 μM). In that context, PAECs were transfected with miR-130a or miR-NC. Treatment with rosiglitazone (Rosi) (FIG. 20C) or forced expression of PPARγ (pPPARγ) (FIG. 20D) reversed the miR-130a-dependent alterations in transcript expression of CCND1, CDKN1A, CDKN2A, and CDKN1B, compared with treatment with rosiglitazone vehicle (DMSO) or transduction with a control transgene (pGFP), respectively. (FIGS. 20E-20H) The same experimental approach was performed in PASMCs. (FIGS. 20E-20F) As in PAECs, PPARγknockdown (FIG. 20F) or miR-130a overexpression (FIG. 20E) decreased CDKN1A, CDKN1B, and CDKN2A expression while increased CCND1 expression as compared with si-NC control. Conversely, inhibition of the miR-130/301 family (tiny-LNA-130) increased CDKN1A, CDKN1B and CDKN2A expression and decreased CCND1 expression as compared with control (tiny-LNA-NC). In PASMCs transfected with miR-130a, pharmacologic activation of PPARγactivity (rosiglitazone 10 μM) (FIG. 20G) or forced expression of PPARγ (pPPARγ) (FIG. 20H) reversed the miR-130a-dependent alterations in transcript expression of CCND1, CDKN1A, CDKN2A, CDKN1B, compared with treatment with rosiglitazone vehicle (DMSO) or transduction with a control transgene (pGFP). In all panels, mean levels in control groups exposed to miR-NC, tiny-LNA-NC, or siRNA control (si-NC) were assigned a fold change of 1, to which relevant samples were compared. In all panels, data are expressed as mean±SD (*P<0.05, ** P<0.01).

FIGS. 21A-21C show that the miR-130/301 family controls the mRNA expression levels of apelin, FGF2, and STAT3. In normoxia (21% O₂) or hypoxia (0.2% O₂, 24 hours), either forced expression of miR-130a mimic versus control (miR-NC) or inhibition of miR-130a (anti-miR-130a) versus inhibition of the entire miR-130/301 family (tiny-LNA-130) versus control (anti-miR-NC and tiny-LNA-NC) was performed in cultured PAECs (FIGS. 21A-21B) or PASMCs (FIG. 21C). In PAECs, RT-qPCR revealed that the miR-130/301 family decreased apelin (APLN) (FIG. 21A) and increased FGF2 (FIG. 21B). In PASMCs, RT-qPCR revealed that the miR-130/301 family increased STAT3 expression (FIG. 21C). In all panels, mean levels in control groups (21% O₂ miR-NC or 21% O₂ Anti-miR-NC) were assigned a fold change of 1, to which relevant samples were compared. In all panels, data are expressed as mean±SD (*P<0.05, ** P<0.01).

FIGS. 22A-22H show that either forced expression of PPARγ or pharmacological activation of PPARγ reverses specific miR-130a-dependent actions in PAECs. In PAECs, forced expression of PPARγ was achieved by lentiviral transduction with a constitutively expressed PPARγ transgene (pPPARγ) that does not encode for a 3′ untranslated region and thus missing the endogenous miR-130/301 binding site. Alternatively, pharmacologic activation of PPARγactivity was achieved by 24 h treatment with rosiglitazone (10 μM). In that context, PAECs were transfected with miR-130a or miR-NC. Forced expression of PPARγ (pPPARγ) or treatment with rosiglitazone (Rosi) reversed the miR-130a-dependent alterations in transcript expression of PPARγ, APLN (FIGS. 22A and 22E), miR-424 and miR-503 (FIGS. 22B and 22F), and FGF2 (FIGS. 22C and 22G) compared with PAECs transduced with a control transgene (pGFP) or treated with rosiglitazone vehicle (DMSO). (FIGS. 22D and 22H) Under these same conditions, immunoblotting revealed corresponding reversal at the protein level of these miR-130a-dependent alterations in expression. In (FIGS. 22A-22C), mean levels in control groups exposed to pGFP and miR-NC were assigned a fold change of 1, to which relevant samples were compared. In (FIGS. 22E-22G), mean levels in control groups exposed to DMSO vehicle and miR-NC were assigned a fold change of 1, to which relevant samples were compared. In all histograms, data are expressed as mean±SD (*P<0.05, ** P<0.01).

FIGS. 23A-23B show that apelin is specifically expressed in PAECs. Comparison of apelin (APLN) expression by RT-qPCR (FIG. 23A) and immunoblotting (FIG. 23B) revealed enriched expression in PAECs but not PASMCs. In (FIG. 23A), mean levels in PASMCs were assigned a fold change of 1, to which relevant levels in PAECs were compared. In (FIG. 23A), data are expressed as mean±SD (*P<0.05, ** P<0.01).

FIGS. 24A-24C show that siRNA knockdown of PPARγ mimics the actions of the miR-130/301 family by increasing STAT3 expression as well as decreasing miR-204 in PASMCs. Transcript levels of PPARγ (PPARγ, left panel-A), STAT3 (right panel-FIG. 24A), and miR-204 (FIG. 24B) were quantified in normoxic (21% O2) PASMCs 48 h after transfection with siRNA against PPARγ (si-PPARγ) or siRNA control. RT-qPCR confirmed PPARγ knockdown after treatment with si-PPARγ (n=3) compared with control (si-NC) (left panel-FIG. 24A). Such PPARγ knockdown led to increased STAT3 (right panel-A), and decreased miR-204 (FIG. 24B). (FIG. 24C) Under these same conditions, immunoblotting revealed a corresponding increase in STAT3 expression at the protein level in the context of PPARγ knockdown. In (FIG. 24A-24B), mean levels in control groups transfected with si-NC were assigned a fold change of 1, to which relevant samples were compared. In (FIGS. 24A-24B), data are expressed as mean±SD (*P<0.05, ** P<0.01).

FIGS. 25A-25F show that either forced PPARγ expression or pharmacological activation of PPARγ reverses specific miR-130a-dependent actions in PASMCs. In PASMCs, forced expression of PPARγ was achieved by lentiviral transduction with a constitutively expressed PPARγ transgene (pPPARγ) that does not encode for a 3′ untranslated region and thus missing the endogenous miR-130/301 binding site. Alternatively, pharmacologic activation of PPARγ activity was achieved by 24-hour (24 h) treatment with rosiglitazone (10 μM). In that context, PASMCs were transfected with miR-130a or miR-NC. Forced expression of PPARγ(pPPARγ) or treatment with rosiglitazone (Rosi) reversed the miR-130a-dependent alterations in transcript expression of PPARγ, STAT3 (FIGS. 25A, 25D), and miR-204 (FIGS. 25B, 25E) compared with PASMCs transduced with a control transgene (pGFP) or treated with rosiglitazone vehicle (DMSO). (FIGS. 25C and 25F) Under these same conditions, immunoblotting revealed corresponding reversal at the protein level of these miR-130a-dependent alterations in expression. In (FIGS. 25A-25B), mean levels in control groups exposed to pGFP and miR-NC were assigned a fold change of 1, to which relevant samples were compared. In (FIGS. 25D-25E), mean levels in control groups exposed to DMSO vehicle and miR-NC were assigned a fold change of 1, to which relevant samples were compared. In all histograms, data are expressed as mean±SD (*P<0.05, ** P<0.01).

FIGS. 26A-26D show that downstream targets of STAT3 are modulated by miR-130/301 in PASMCs. (FIGS. 26A-26D) Transcript levels of the STAT3 targets NFATC2 and PIM1 were quantified in PASMCs 48 h after transfection with (FIG. 26A) miR-130a or tiny-LNA-130 versus appropriated controls (miR-NC and tiny-LNA-NC, respectively) or with (FIG. 26B) siRNA specific for PPARγ (si-PPARγ) versus siRNA control (si-NC). Consistent with the modulation of STAT3 expression and activity, PPARγ knockdown or miR-130a overexpression increased NFATC2 and PIM1 expression as compared with controls. Conversely, inhibition of the miR-130/301 family (tiny-LNA-130) decreased NFATC2 and PIM1 expression as compared with control (tiny-LNA-NC). (FIGS. 26C-26D) In PASMCs, pharmacologic activation of PPARγactivity was achieved by 24 h treatment with rosiglitazone (10 μM) (FIG. 26C). Forced expression of PPARγ was achieved by lentiviral transduction with a constitutively expressed PPARγtransgene (pPPARγ) (FIG. 26D). Treatment with rosiglitazone (Rosi) or forced expression of PPARγ (pPPARγ) reversed the miR-130a-dependent alterations of NFATC2 and PIM1 as compared with PASMCs treated with rosiglitazone vehicle (DMSO) or transduced with a control transgene (pGFP). In all panels, mean levels in control groups exposed to miR-NC or siRNA control (si-NC) were assigned a fold change of 1, to which relevant samples were compared. In all panels, data are expressed as mean±SD (*P<0.05, ** P<0.01).

FIG. 27 is a protocol for forced miR-130a expression in the pulmonary tissue and pulmonary vasculature of mice. In the presence of SU5416 (days of injection are highlighted in red) but in the absence of hypoxia, intrapharyngeal injection of wildtype mice was performed serially 4 times at 1 week intervals with 1 nmol of miR-NC or 1 nmol of miR-130a (days of administration are in black). During this period, mice were treated daily by oral gavage with 20 mg/kg of rosiglitazone or vehicle. At day 25, echocardiography and right heart catheterization was performed followed by euthanasia and blood/tissue sampling.

FIGS. 28A-28C shows that intrapharyngeal injection of miR-130a oligonucleotide is restricted to delivery to the pulmonary tissue and pulmonary vasculature of mice. (FIG. 28A) By RT-qPCR, expression levels of miR-130/301 family members were analyzed in whole lung lysate of mice that underwent serial intrapharyngeal injection of miR-130a or miR-NC oligonucleotides either in the presence or absence of rosiglitazone (n=8 mice/group). Only expression of miR-130a was significantly increased by miR-130a administration. For each miRNA, mean levels in control groups (miR-NC) were assigned a fold change of 1, to which relevant samples were compared. (FIG. 28B) Formalin-fixed paraffin-embedded tissue sections of mouse lung treated as (FIG. 28A) were analyzed by in situ hybridization using probes recognizing miR-130a or scrambled control. Microscopy revealed an increase of staining intensity for miR-130a signal particularly in small pulmonary arterioles. Quantification of staining intensity (right panel) of small pulmonary arterioles (<100 μm diameter, 10 vessels per mouse) revealed significantly and specifically increased miR-130a expression after miR-130a administration. (FIG. 28C) By RT-qPCR, miR-130a expression was not altered in heart, liver, kidney, or spleen after serial intrapharyngeal administration of either miR-130a or miR-NC in the presence or absence of rosiglitazone (n=7 mice/group). For each organ, mean levels in control groups (miR-NC) were assigned a fold change of 1, to which relevant samples were compared. In all panels, data are expressed as mean±SEM (*P<0.05, ** P<0.01).

FIGS. 29A-29C show that left ventricular function is unchanged in mice administered miR-130a. By echocardiography, left ventricular ejection fraction (FIG. 29A), fractional shortening (FIG. 29B), and interventricular septal thickness at diastole (IVsd, FIG. 29C) were unchanged in mice administered miR-130a compared with miR-NC, either in the presence or absence of rosiglitazone (n=8 mice/group). Data are expressed as mean±SEM (*P<0.05, ** P<0.01).

FIGS. 30A-30E show that forced expression of miR-130a induces PH via at least a partially PPARγ-dependent manner in vivo. (FIG. 30A) As assessed by measuring the mass ratio of right ventricle/(left ventricle+septum), right ventricular remodeling was increased by serial intrapharyngeal delivery of miR-130a to mice (miR-130a, n=11 mice) as compared with control (miR-NC, n=13 mice). A non-statistically significant trend toward reversal of such right ventricular pathology was observed with rosiglitazone treatment (miR-NC+Rosi, n=9 mice; miR-130a+Rosi; n=9). (FIG. 30B) Pulmonary arteriolar density was assessed in paraffin embedded lung sections after vascular endothelial CD31-staining. The number of vessels (<100 μm) was counted in 30 high-power fields (HPF; 400×) per lung, in 8 animals per group. The density of pulmonary arterioles was decreased in mice treated with miR-130a as compared with mice treated with miR-NC. This decrease was reversed by rosiglitazone treatment. (FIG. 30C) The percentage of muscularized small (<100 μm diameter) pulmonary arterioles in the lungs from mice treated as in (FIG. 30A) increased with miR-130a administration. Such remodeling was reversed to baseline levels by rosiglitazone treatment. (FIGS. 30D and 30E) Confirming the results of FIG. 7D, immunoblotting (FIG. 30D) and quantification (FIG. 30E) from whole lung lysates revealed decreased PPARγ expression and increased Stat3 phosphorylation in mice administered miR-130a (n=5 mice) compared with miR-NC (n=4 mice). Actin was used as a loading control. Data are expressed as mean±SEM (*P<0.05, ** P<0.01).

FIGS. 31A-31E show that severity of PH in mice is comparable after either forced expression of miR-130a alone or chronic exposure to hypoxia. Wildtype mice were exposed to chronic hypoxia (10%02) for 3 weeks (n=6). In parallel, in the absence of hypoxia, intrapharyngeal injection of wildtype mice was performed serially 4 times at 1 week intervals with 1 nmol of miR-NC scrambled control (n=6) or 1 nmol of miR-130a (n=8). After 3 weeks, indices of PH were assessed by measuring, right ventricular systolic pressure (RVSP) (FIG. 31A), right ventricular remodeling as reflected by the mass ratio of right ventricle/(left ventricle+septum) (FIG. 31B), the density of pulmonary arterioles (FIG. 31C), pulmonary arteriolar remodeling [via immunohistochemical (IHC) staining of <100 μm diameter pulmonary vessels for α-smooth muscle actin (α-SMA)] (FIG. 31D), and the percentage of muscularized small (100<μm diameter) pulmonary arterioles (FIG. 31E). Both hypoxia and miR-130a similarly increased RVSP (FIG. 31A), ventricular remodeling (FIG. 31B), pulmonary arterioles remodeling (FIG. 31D), and the percentage of muscularized small pulmonary arterioles (FIG. 31E). Conversely, both hypoxia and miR-130a similarly decreased the density of pulmonary arterioles (FIG. 31C) compared with controls (normoxia (21% O₂; n=6) and administration of miR-NC, respectively). Data are expressed as mean±SEM (*P<0.05, ** P<0.01). Scale bar 50 μm.

FIG. 32 is a protocol for pharmacologic inhibition of the miR-130/301 family in the pulmonary tissue and pulmonary vasculature of mice in order to reverse PH progression in vivo. Wildtype mice were exposed to chronic hypoxia+SU5416. After two weeks of exposure and confirmation of PH by increased RVSP (Ctrl 2 weeks, n=5 mice), exposure to chronic hypoxia+SU5416 was continued in a separate cohort of mice for another 2 weeks, accompanied by serial intrapharyngeal injections (every 4 days for a total of 3 doses) with 10 mg/kg of control oligonucleotide (Short-NC, n=7 mice), “shortmer” oligonucleotide recognizing the seed sequence of the miR-130/301 family (Short-130, n=7 mice), or PBS (n=4 mice).

FIGS. 33A-33B show that Intrapharyngeal injections of shortmers are restricted to delivery to the pulmonary tissue and pulmonary vasculature of mice. (FIG. 33A) Intrapharyngeal injections of Cy5-labeled tiny-oligonucleotide (10 mg/kg) were performed in mice (n=10 mice). After two days, mice were euthanized and cryosections were obtained from from lung, heart and liver. Vascular endothelium was identified by anti-CD31 staining (green), and Cy5-labeled shortmer was localized by red stain. Delivery of shortmer was confirmed specifically in pulmonary vessels of the lung but not the heart or liver. (FIG. 33B) To directly assess the localization of the delivered oligonucleotide in the lung of injected mice, antibodies were developed recognizing the oligonucleotide modifications in the shortmer backbone (see Methods). Immunohistochemistry (IHC) of three serial sections of mouse lung injected with PBS (n=4) or Short-130 (n=7) were performed using antibody against α-SMA (smooth muscle cells), CD31 (endothelial cells) and shortmer oligonucleotide (Short-130). Shortmer oligonucletide labeling revealed that Short-130 appears to localize in both endothelial and smooth muscle cells. Scale bar 50 μm.

FIGS. 34A-34C show that left ventricular function is unchanged in mice administered Short-130. By echocardiography, left ventricular ejection fraction (FIG. 34A), fractional shortening (FIG. 34B), and interventricular septal thickness at diastole (IVsd) (FIG. 34C) were unchanged in mice exposed to hypoxia+SU5416 (4 weeks) and administered Short-130 compared with Short-NC (n=5 mice/group). Data are expressed as mean±SEM (*P<0.05, ** P<0.01).

FIGS. 35A-35E show that inhibition of the miR-130/301 family by Short-130 reverses multiple molecular, histologic, and hemodynamic indices of experimental PH in mice in vivo. (FIG. 35A) In the context of pre-existing PH in mice caused by chronic hypoxia+SU5416, as assessed by measuring the mass ratio of right ventricle/(left ventricle+septum), right ventricular remodeling was decreased by serial intrapharyngeal delivery of Short-130 (n=7 mice) as compared with control (Short-NC; n=7 mice) or PBS treatment (PBS; n=4 mice) where right ventricular remodeling increased over time. (FIG. 35B) The density of pulmonary arterioles progressively decreased after two weeks (Ctrl 2 weeks) and four weeks of hypoxia+SU5416 exposure (Short-NC) as compared with control mice (normoxia+SU5416). Treatment with Short-130 prevented the decline of pulmonary arteriolar density as compared with control (Short-NC). (FIG. 35C) The percentage of muscularized small (100<μm diameter) pulmonary arterioles in the lungs from mice treated as in (FIG. 35A) decreased with Short-130 administration (n=7 mice) as compared with control (Short-NC; n=7 mice), where arteriolar muscularization increased over time. (FIG. 35D) Confirming the results of FIG. 8D, immunoblotting (left panel) and quantification (right panel) from whole lung lysates revealed increased PPARγ expression and decreased Stat3 phosphorylation in mice administered Short-130a (n=5 mice) compared with Short-NC (n=4 mice). Actin was used as a loading control. Data are expressed as mean±SEM (*P<0.05, ** P<0.01).

FIGS. 36A-36B show that the miR-130/301 family is predicted to regulate several functional pathways in the PH network. (FIG. 36A) The miR-130/301 family targets 28 members of the PH network (blue nodes). Together with their first degree interactors (gray nodes), this subnetwork spans 177 nodes, 71% of the PH network. (FIG. 36B) MiR-130/301 family targets and their first degree interactors within the PH network cover several broad functional modules. Genes were cross-referenced with 26 PH-relevant pathways defined by the union of multiple functionally related gene sets in the KEGG, Biocarta, Reactome, and NCI PID pathway databases (see also Table 7).

FIG. 37A-37D show that the miR-130/301 family regulates the production of vasoactive factors in PAECs. In normoxia (21% O2) or hypoxia (0.2% O2, 24 h), either forced expression of miR-130a mimic versus control (NC) or inhibition of miR-130a (anti-miR-130a) versus inhibition of the entire miR-130/131 family (tiny-LNA-130) versus control (NC) was performed in cultured PAECs (FIGS. 37A-37D). RT-qPCR (A,B) and immunoblotting (FIG. 37C) revealed that forced miR-130a expression increased VEGFA (FIGS. 37A,37C) and EDN1 (FIGS. 37A,37C), and decreased NOS3 (FIGS. 37A,37C). Conversely, inhibition of the entire miR-130/301 family (tiny-LNA-130) reversed these gene expression changes compared with control (tiny-LNA-NC), particularly evident at the transcript level in hypoxic PAECs (B) and at the protein level in normoxic PAECs (FIG. 37C). In most cases, inhibition of the miRNA family was more effective than inhibition of miR-130a alone (anti-miR-130a,) versus control (anti-miR-NC) thus demonstrating the importance of the coordinated actions of this miRNA family. (FIG. 37D) As assessed by ELISA, transfection by miR-130a (left graph) increased mature endothelin-1 expression in conditioned media of PAECs during normoxia (21% O₂) and to a greater extent during hypoxia (0.2% O₂). Alternatively (right graph), in hypoxia (0.2% O₂) but not normoxia (21% O₂), inhibition of the entire miR-130/301 family (tiny-LNA-130) decreased endothelin-1 expression to a greater extent than inhibition of miR-130a alone (anti-miR-130a). In (FIGS. 37A,37B), mean expression in control groups (miR-NC, anti-miR-NC, tiny-LNA-NC) was assigned a fold change of 1, to which relevant samples were compared. In all histograms, data are expressed as mean+/−SD (*P<0.05, ** P<0.01).

FIGS. 38A-38B show that Endothelin-1 is overexpressed and correlates with the expression of miR-130/301 family members in plasma of PH patients. (FIG. 38A) Plasma levels of mature endothelin-1 were positively correlated with plasma levels of miR-130/301 family members in patient samples. Here, 5 non-PH controls (mPAP<25 mmHg) were compared with two groups of PH patients stratified by hemodynamic severity-7 patients with mPAP between 25 to 45 mmHg and 7 patients with mPAP>45 mmHg. (FIG. 38B) Plasma levels of mature endothelin-1 progressively increased with hemodynamic severity of PH, and patient stratification was performed as above.

FIGS. 39A-39E show that The miR-130/301 family modulates the production of specific vasoactive factor through PPARγ in PAECs. (FIG. 39A) RT-qPCR revealed that miR-130a-dependent expression changes in EDN1 and NOS3, but not VEGFA, in PAECs were reversed by lentiviral transduction of a PPARγ (pPPARγ) transgene as compared with control (pGFP). In this context, normoxic PAECs were transfected with miR-130a or miR-NC. (FIG. 39B) Pharmacologic activation of PPARγ activity was achieved by 24 h treatment with rosiglitazone (10 μM). Rosiglitazone treatment (Rosi) as compared with vehicle (DMSO) reversed the miR-130a-dependent alterations in transcript expression of NOS3 and EDN1 but not VEGFA. (FIG. 39C) Similarly, transcript levels of VEGFA, EDN1, and NOS3 were quantified in normoxic (21% O2) or hypoxic (0.2% O2) PAECs 48 h after transfection with siRNA against PPARγ (si-PPARγ) or siRNA control (si-NC). Under both normoxia and hypoxia, such PPARγknockdown altered EDN1 and NOS3, but not VEGFA, expression compared with si-NC. (FIG. 39D) Under the same condition as (FIGS. 39A-39C), immunoblotting revealed corresponding alterations in protein-level expression of EDN1, NOS3, and VEGFA following forced expression of PPARγ, treatment with rosiglitazone, and transfection with si-PPARγ. (FIG. 39E) As assessed by ELISA, secreted EDN1 levels in conditioned media from PAECs treated as in (FIGS. 39A-39C) were decreased following lentiviral transduction of PPARγ and in the presence of rosiglitazone, and increased 48 h after transfection with siPPARγ. In (FIGS. 39A-39B), mean expression in control groups (miR-NC and si-NC) was assigned a fold change of 1, to which relevant samples were compared. In all histograms, data are expressed as mean+/−SD (*P<0.05, ** P<0.01).

FIGS. 40A-40C show that endothelin-1 activates STAT3 phosphorylation and actinomyosin-dependent contraction of PASMCs. (FIG. 40A) A schematic diagram of co-culture experiments: conditioned media from transfected PAECs (miRNA, anti-miRNA, tiny-LNA or siRNA under normoxic or hypoxic conditions) were filtered and applied onto naïve PASMCs cultured in matrigel. At specific times, PASMCs or PASMC-matrigel cultures were arrested and analyzed for STAT3 activation (phosphorylation) or contraction, respectively. (FIG. 40B) Immunoblotting revealed that exposure of PASMCs to endothelin-1 led to a time-dependent increase of STAT3 phosphorylation (P-STAT3). (FIG. 40C) As a reflection of PASMC contractile function, application of endothelin-1 into a PASMC culture in matrigel for 4 days led to matrigel contraction as compared with vehicle control.

FIGS. 41A-41E show that The miR-130/301/PPARγ/EDN1 axis in PAECs induces paracrine activation of STAT3 and actinomyosin-dependent contraction of PASMCs. (FIG. 41A) PASMCs were treated with conditioned media from transfected PAECs (miRNA, anti-miRNA, tiny-LNA or siRNA under normoxic or hypoxic conditions). STAT3 phosphorylation increased in the context of miR-130a-expressing and PPARγ-silenced PAECs (normoxic conditioning, left and central panels) and decreased in the context of miR-130/301 inhibition (hypoxic conditioning, right panels). (FIG. 41B) The miR-130a-dependent increase of STAT3 phosphorylation in PASMCs was reversed by lentiviral transduction of a PPARγ (pPPARγ) transgene as compared with control (pGFP) in PAECs (upper panels) or by PPARγ activation via rosiglitazone in PAECs (lower panels). (FIG. 41C) PASMCs were treated as in (FIG. 41A) in the presence (+) or absence (−) of the endothelin receptor antagonist ambrisentan (10 μM). Notably, ambrisentan prevented STAT3 activation in the setting of either miR-130a expression in PAECs, thus demonstrating the downstream importance of EDN1 in such PASMC activation. (FIGS. 41D and 41E) Matrigel contraction was analyzed after treatment of PASMCs as in ((FIGS. 41A, 41B, and 41C), demonstrating that PASMC contraction is directly correlated with the observed changes in STAT3 phosphorylation. In all histograms, data are expressed as mean+/−SD (*P<0.05, ** P<0.01).

FIGS. 42A-42F show that The miR-130/301/PPARγ axis regulates endothelin-1 expression in mice suffering from experimental PH. (FIGS. 42A-42C) Serial intrapharyngeal delivery of miR-130a mimic oligonucleotide was used to force miR-130a expression in whole lung, as compared with control mimic (miR-NC), as described in Bertero et al., “Systems-level regulation of microRNA networks by miR-130/301 promotes pulmonary hypertension” (submitted manuscript). (FIG. 42A) Immunohistochemical (IHC) staining of <100 μm diameter pulmonary vessels was performed to quantify EDN1 levels. Ten vessels (<100 μm) were quantified for each mouse (bar graphs). Consistent with the regulatory mechanisms delineated in cell culture (FIGS. 37-41), miR-130a expression increased EDN1. (FIG. 42B) By ELISA, endothelin-1 in plasma was up-regulated by miR-130a and reversed to baseline level after rosiglitazone treatment. (FIG. 42C) Confirming the results of (FIG. 42A), immunoblotting from whole lung lysates revealed increased EDN1 in mice administered miR-130a (n=5 mice) compared with miR-NC (n=4 mice). (FIGS. 42D-42F) As described in Bertero et al., “Systems-level regulation of microRNA networks by miR-130/301 promotes pulmonary hypertension” (submitted manuscript), mice were exposed to chronic hypoxia accompanied by injection of SU5416 in order to induce PH. After two weeks of exposure and documented development of PH (Cntrl 2 weeks) as compared with mice exposed to normoxia+SU5416 (Normoxia), mice were treated for an additional two weeks of hypoxia+SU5416, accompanied by serial intrapharyngeal injections with a tinymer inhibitor of the miR-130/301 family (Short-130) or control inhibitor (Short-NC). (FIG. 42D) Immunohistochemical (IHC) staining was performed as in (FIG. 42A), showing decreased EDN1 expression when miR-130/301 expression was inhibited in the context of hypoxia+SU5416. (FIG. 42E) By ELISA, EDN1 in plasma was up-regulated in disease (Ctrl 2 weeks, PBS, and tiny-NC) but was decreased by Short-130. (FIG. 42F) Immunoblotting from whole lung lysates revealed decreased EDN1 in mice administered tiny-130a (n=5 mice) compared with miR-NC (n=4 mice). Data are expressed as mean+/−SEM (*P<0.05, ** P<0.01). Scale bar 50 μm.

FIGS. 43A-43G shows collagen expression and cross-linking are abnormal in multiple PH models and human patients. A-C) Mice were treated with or without the VEGF receptor antagonist SU5416 in combination with chronic hypoxia (10% O₂ for 3 weeks, n=12) in order to induce PH. Picrosirius red coloration of formalin-fixed paraffin-embedded mouse lung was imaged in parallel (top) or orthogonal (bottom) light and quantification of <100 nm pulmonary vessels (10 vessels per mouse) (A) revealed an increase in collagen expression (top) and collagen cross-linking (bottom) under disease conditions. RT-PCR (B) confirmed an increase in the expression of collagen and the collagen cross-linking gene Lox in diseased lung. At the protein level, the soluble and insoluble (cross-linked) collagen content of the lung were assessed via Sircol assay (C) and confirmed the increase in collagen expression and cross-linking in diseased mice (n=5 per group). D-F) Rats were treated with monocrotaline (3 weeks, n=8 per time point) in order to induce PH. Picrosirius red coloration of rat lung and quantification of <100 nm pulmonary vessels (10 vessels per rat) (D) demonstrated increased collagen expression (top) and collagen cross-linking (bottom), corresponding with increased disease severity (as measured by RVSP n=4 per group) (E) over time. Increased collagen concentration and collagen cross-linking in the lung was also confirmed by Sircol assay (F). G) Picrosirius red coloration of human of human lung and quantification of <200 μm pulmonary vessels (10 vessels per patient) confirmed that collagen expression and cross-linking are increased in the lungs of PH patients.

FIGS. 44A-44I shows MiR-130/301 family expression is increased by matrix stiffness in a YAP/TAZ- and OCT4-dependent fashion. A-E) Pulmonary artery adventitial fibroblasts (PAAF) were cultured on collagen coated hydrogels of varying stiffness. Expression of miR-130/301 family members was increased by matrix stiffness, as quantified by RT-PCR (A). SiRNA knockdown of the YAP and TAZ transcription factors reversed the stiffness-mediated upregulation of miR-130/301 family members (B) and of the transcription factors POU5F1 (OCT4) as well as CTGF a direct target of YAP/TAZ (C). SiRNA knockdown of POU5F1 (OCT4) did not impact stiffness-mediated upregulation of CTGF (D), but reversed stiffness-mediated upregulation of the miR-130/301 family (E). F-I) Rats were treated with monocrotaline (3 weeks, n=8 per time point) in order to induce PH (see also FIG. 43). Serial sections of rat lung were stained for miR-130a, YAP1, and OCT4 (F). Quantification of miR-130a stain intensity (G) and percentage of positively stained cells (H), revealed a correlation between miR-130a, Yap1, and Oct4 expression (dark arrows indicate the presence of stain, light arrows indicate the absence of stain). I) Serial section staining of human lung demonstrated a similar correlation between miR-130a and YAP1 expression in the lungs of PH patients.

FIGS. 45A-45K show miR-130/301 family upregulates MMP2 via downregulation of TIMP2. A) Sequence alignment between miR-130a and the 3′ UTR of TIMP2 highlights a highly conserved putative binding site 5′-UUGCACU-3′ (SEQ ID NO: 15). Sequences shown are ACCCUUGGUAGGUAUUAGACUUGCACUUU (SEQ ID NO: 16)), GCCUUUCGUAGCAUUAGACUUUGCACUUU (SEQ ID NO: 17)), GCCUUUUGUAGCAUUAGACUUUGCACUUU (SEQ ID NO: 18)), UCCCCCGGUAGCUGUUAGACUUGCACUUU (SEQ ID NO: 19), and has-miR-130a (SEQ ID NO: 4). SEQ ID NOs: 16-19 are shown in the 5′->3 direction (left to right) and SEQ ID NO: 4 is shown in the 3′->5′ direction (left to right). B) A luciferase reporter assay confirmed the recognition of the binding site in the TIMP2 transcript by miR-130a. HEK293T cells were transfected with a luciferase reporter construct carrying a 3′ UTR with either the predicted binding site for the miR-130/301 family encoded by the human TIMP2 transcript (WT) or scrambled mutant (mut). Cells were transfected with oligonucleotide mimics of miR-130a or control (miRNC). miR-130a reduced Renilla luciferase activity as compared with miR-NC in the setting of the WT binding site, but induced no significant change in the setting of the mutant binding site. Luciferase levels in cells transfected with miR-NC were normalized to 1, to which other conditions were compared. C). In three dimensional cell culture, MMP2 activation was increased by matrix stiffness in a miR-130/301 dependent fashion, as was downregulation of the MMP2 inhibitor TIMP2. D-E) Both stiffness and miR-130a overexpression were observed to upregulate collagen, CTGF and LOX (D), and these changes were reduced by inhibition of the miR-130/301 family (E). F) The collagen-content of conditioned media was increased both by stiffness and miR-130a overexpression. Conversely inhibition of miR-130/301 prevents matrix stiffness induced collagen secretion. G) In 3D cell culture knockdown of TIMP2 in PAAF by siRNA mimics the miR-130/301 effects on MMP2 activation. H-I) However, miR-130/301-dependent upregulation of collagen, CTGF, and LOX (H), as well as the collagen content of media (I), were observed to be independent of TIMP2 expression. J) In situ staining of mouse lung demonstrated that both downregulation of Timp2 and upregulation of Mmp2 were induced in hypoxia treated mice. (K) In situ staining of human lung confirmed downregulation of TIMP2, and upregulation of MMP2, in the lungs of PH patients.

FIG. 46A-46G show miR-130/301-mediated regulation of collagen expression and cross-linking is dependent on the PPARγ-APOE axis. A) Constitutive expression of PPARγreversed miR-130/301 mediated upregulation of collagen and LOX. B) Cells exposed to growth media containing APOE were resistant to miR-130a-induced upregulation of collagen, CTGF, and LOX. C) HEK293T cells were transfected with a luciferase reporter construct carrying a 3′ UTR with either the predicted binding site for the miR-130/301 family encoded by the human LRP8 transcript (WT) or scrambled mutant (mut). Cells were transfected with oligonucleotide mimics of miR-130a or control (miRNC) (see also FIG. 45B). miR-130a reduced Renilla luciferase activity as compared with miR-NC in the setting of the WT binding site, but induced no significant change in the setting of the mutant binding site. Luciferase levels in cells transfected with miR-NC were normalized to 1, to which other conditions were compared. D) RT-PCR confirmed that LRP8 expression is reduced by miR-130a and recovered by inhibition of the mIR-130/301 family.

FIGS. 47A-47F show miR-130/301-induced fibrosis is reversed by the LOX inhibitor BAPN. In the presence of SU5416 (days of injection are highlighted in red) but in the absence of hypoxia, wildtype mice received 4 serial intrapharyngeal injections at 1 week intervals with 1 nmol of miR-NC or 1 nmol of miR-130a. During this period, mice were treated with 30 mg/kg/day of BAPN in drinking water. At day 25, echocardiography and right heart catheterization was performed, followed by euthanasia and blood/tissue sampling. The LOX inhibitor BAPN lessened the mIR-130/301-mediated increase in disease severity, as quantified by RVSP (A), extent of right ventricular hypertrophy (B), and extent of pulmonary arteriole muscularization (C). D) Picrosirius red coloration and in situ staining of mouse lung revealed a decrease in miR-130a-mediated expression changes in collagen, smooth muscle actin, Yap1, and to a lesser extent Mmp2, as well as a renormalization of collagen cross-linking, when mice were treated with BAPN. RT-PCR (E) confirmed an increase in the expression of collagen and the collagen cross-linking gene Lox in diseased lung (miR-130a) compared to control lung (miR-NC) that were temper in mice treated with BAPN (miR-130+BAPN) compared to control (miR-130). F) Biochemical analysis of collagen in mice lung (n=5 per group) confirmed that miR-130a-induced collagen (Soluble) and collagen cross-linking (Insoluble) were reversed by BAPN treatment.

FIGS. 48A-48J show inhibition of the miR-130/301 family tempers the development of fibrosis. A) Wildtype mice were exposed to chronic hypoxia+SU5416. After two weeks of exposure and confirmation of PH as quantified by increased RVSP (Ctrl 2 weeks, n=5 mice), exposure to chronic hypoxia+SU5416 was continued in a separate cohort of mice for another 2 weeks, accompanied by serial intrapharyngeal injections (every 4 days for a total of 3 doses) with 10 mg/kg of control oligonucleotide (Short-NC, n=7 mice) or “shortmer” oligonucleotide recognizing the seed sequence of the miR-130/301 family (Short-130, n=7 mice). Short-130 oligonucleotide delivery reverted collagen deposition and cross-linking, as demonstrated by Picrosirius red coloration, decreased Mmp2 expression levels, nuclear Yap1 localization, and increased Timp2 and Lrp8 expression levels. RT-PCR (B) confirmed a decrease in expression of collagen and the collagen cross-linking gene Lox in Short-130 treated lung. Moreover the expression level of Ctgf, a direct target of Yap1, was also decreased. At the protein level, soluble collagen and insoluble (cross-linked) collagen of the lung were assessed via Sircol assay (C), demonstrating a decrease in collagen expression and cross-linking in Short-130 treated mice, as compared to controls. D) In Bleomycin-induced lung fibrosis in mice, expression levels of the miR-130/301 family were assess by RT-PCR. 14 days (n=10) or 21 days (n=10) after bleomycin injection, expression levels of the miR-130/301 family were significantly increased in the lungs of Bleomycin-treated mice, as compare to mice injected with PBS (n=9). E) Serial sections of mouse lung were stained for miR-130a and Yap1. Quantification of miR-130a stain intensity and percentage of positively stained cells revealed a correlation between miR-130a and Yap1 nuclear localization. F) A protocol was performed to determine whether a “shortmer” inhibitor of miR-130/301 (short-130) would prevent lung fibrosis in Bleomycin-treated mice. Following Bleomycin treatment, mice were treated every 2 days with an intra-peritoneal injection of Short-NC (n=8) or Short-130 (n=10) (20 mg/kg) for a 21 day period. Lung fibrosis was quantified by α-SMA staining, collagen staining (PIcrosirius Red), and (G) Ashcroft score. Treatment with Short-130 decreased α-SMA expression and collagen deposition and cross-linking, resulting in a decrease in Yap1 nuclear localization. RT-PCR (H) confirmed a decrease in the expression of collagen and the collagen cross-linking gene Lox. Moreover, a decrease in Ctgf (a direct target of Yap) was also observed. I) Biochemical analysis of collagen in mice lung confirmed a decreased of Soluble collagen and insoluble collagen in mice treated with Short-130 (n=10) compared to control mice (n=8). Finally, RT-PCR (J) and in situ hybridization (K) confirmed the increase in miR-130/301 family expression in the lungs of human fibrosis patients. In these patients, a correlation between miR-130a expression, Yap1 expression, and collagen cross-linking was also found.

FIGS. 49A-49F show collagen expression and cross-linking are elevated in multiple models of PH. Collagen expression (A,C,E) and cross-linking (B,D,F) were assessed in VHL−/− mice (A-B), transgenic (TgIL6) mice overexpressing IL6 (C-D), and rats treated with SU5416 in combination with chronic hypoxia (10% O₂, 3 weeks) (E-F).

FIGS. 50A-50L show tissue mechanics regulate the expression of PH-relevant miRNA though YAP/TAZ activation. Pulmonary artery smooth muscle cells (PASMCs), pulmonary artery endothelial cells (PAECs), and pulmonary artery adventitial fibroblasts (PAAF) were cultured on collagen coated hydrogels of varying stiffness (see also FIG. 44). A) Cell-type specificity of PH-relevant miRNA. B-D) Matrix stiffening induced the expression of miR-21 and miR-27a in PASMCs (B) and PAAFs (D), and of miR-424 in PAECs (C). E-F) Stiffness-mediated upregulation of miR-21 and miR-27a in PAAFs was observed to depend on YAP/TAZ expression (E) but was independent of POU5F1 (OCT4) expression (F). G-L) Expression of the mIR-130/301 family is induced by stiffness (G,J), and is dependent on both YAP/TAZ (H,K) and POU5F1 (OCT4) (I,L) expression in both PAECs and PASMCs.

FIGS. 51A-51D show miR-130/301 family modulates ECM proteins in PAECs and PASMCs. A) MMP2 activation and TIMP2 downregulation were induced by miR-130a overexpression and reversed by inhibition of the miR-130/301 family in PAECs and PASMCs. B) Collagen, CTGF, and LOX were observed to be upregulated by miR-130a overexpression in PAECs and PASMCs. C) Stiffness-mediated upregulation of collagen, CTGF, and LOX were reversed by inhibition of the miR-130/301 family in PAECs and PASMCs. D) The collagen content of conditioned media was increased by both stiffness and by miR-130a overexpression, and reversed by inhibition of the miR-130/301 family.

FIGS. 52A-52D show inhibition of collagen cross-linking tempers PH development. A) Prevention and rescue treatment model. B-E) BAPN both prevented and reversed disease severity as quantified by (B) RVSP, (C) extent of right ventricular hypertrophy, (D) expression of collagen, Yap1, and smooth muscle actin, as well as collagen cross-linking, and (E) extent of muscularization. F) Soluble and insoluble (cross-linked) collagen content of the lung was assessed via Sircol assay (n=5 per group) and demonstrated that collagen concentration was normalized by BAPN rescue treatment, and kept at baseline by preventative BAPN treatment.

FIGS. 53A-53D show inhibition of the miR-130/301 family tempers PH development and vessel fibrosis in monocrotaline treated rats. A protocol was performed to determine whether a “shortmer” inhibitor of miR-130/301 (short-130) would prevent PH development in monocrotaline treated rats. Following monocrotaline treatment, rats were treated every 2 days with an intra-peritoneal injection of Short-NC (n=8) or Short-130 (n=8) (20 mg/kg) for a 16 day period. At day 16, right heart catheterization was performed, followed by euthanasia and blood/tissue sampling.) and collagen cross-linking (insoluble) were reversed by Short-130 treatment. Delivery of Short-130 lessened the mIR-130/301-mediated increase in disease severity, as quantified by RVSP (A) and extent of right ventricular hypertrophy (B). C) Picrosirius red coloration and in situ staining of mouse lung revealed a decrease in Monocrotaline-mediated expression changes in collagen, smooth muscle actin, and Yap1, as well as a renormalization of collagen cross-linking, when rats were treated with Short-130. D) Biochemical analysis of collagen in mice lung (n=5 per group) confirmed that Monocrotaline-induced collagen (soluble) and collagen cross-linking (insoluble) were reversed by Short-130 treatment.

FIGS. 54A and 54B show miR-130/301 targets are down regulated in bleomycine-induced lung fibrosis mice model, and rescue by Short-130 treatment. A) In Bleomycin-induced lung fibrosis in mice, expression levels of Timp2, Pparγ and Lrp8 were decreased 14 days and 21 days after bleomycin injection compared to PBS treated mice as quantified by IHC. B) However, 21 days after bleomycin injection expression level of Timp2, Pparγ and Lrp8 were sustained in mice treated with Short-130 (n=10) compared to mice treated with Short NC (n=8).

FIG. 55 shows positive correlation between miR-130a expression, stiffness and Yap1 activation in mice model of lung fibrosis. Serial sections of mouse lung treated with Bleomycin were stained for Collagen (Picrosirius Red) miR-130a and Yap1. Quantification of miR-130a stain intensity and percentage of positively stained cells revealed a correlation between miR-130a and Yap1 nuclear localization and Collagen crosslinking.

FIGS. 56A-56I show pulmonary arteriolar stiffening is a hallmark of PH. A-C) Mice were exposed to hypoxia+/−SU5416 (10% O₂ for 3 weeks, n=12/group) in order to induce PH. A) Picrosirius Red stain of mouse lung tissues was imaged in parallel light to display total collagen content (top) or orthogonal light to display collagen crosslinking (bottom) (<100 μm vessel diameter; 10 vessels/animal). B) Increased total collagen and crosslinking (decreased Soluble/Insoluble ratio) in PH lung were demonstrated by Sircol assay (n=5/group). C) RT-qPCR revealed an increase in collagen isoforms and the collagen crosslinking gene LOX in PH lung (n=8-12 per group). D-H) Monocrotaline was administered to rats (3 weeks, n=6-8/time point) to induce PH. Picrosirius Red stain (D) and Sircol assay (E) revealed increased peri-arteriolar collagen crosslinking (starting at D3) evident prior to hemodynamic (right ventricular systolic pressure, RVSP) (F) and histologic (vessel thickness, <100 μm vessel diameter) (G) disease. H) RT-qPCR revealed an increase in CTGF, collagen isoforms, and LOX in PH rat lung (n=8/group). I) By Picrosirius Red stain of human lung, quantification of <200 μm pulmonary vessels (10 vessels/patient) revealed increased perivascular collagen and collagen crosslinking in PAH (controls n=8; PAH n=19). See also FIGS. 63A-63H. Data are expressed as mean±SEM (*P<0.05; ** P<0.01).

FIGS. 57A-57M show ECM stiffening induces miR-130/301 for downstream modulation of collagen deposition and crosslinking through a PPARγ-ApoE-LRP8 axis. A) A fibrosis network, composed of known fibrotic genes and their closest first degree interactors (left, Tables 9 and 10) shares a large portion of its members with a PH disease network [right, as previously described [(Bertero et al., 2014b)]. Color-coding denotes architectural network clusters. Enlarged nodes are shared by both networks, and encircled genes are miR-130/301 direct targets (per Targetscan 6.2), thus highlighting a prominent fibrotic component among the miR-130/301 targets. miR-130/301 was ranked among the top five miRNA by “spanning score” [see Experimental Procedures (Bertero et al., 2014b)] in both network contexts, reflecting the functional overlap and this miRNA family's shared, systems-level control over both networks. B-C) miR-130/301 expression was quantified in human PAAFs cultured in hydrogel of varying stiffness (B) and transfected with siRNAs (YAP/TAZ versus si-NC control) (C). D) miR-130/301 was measured in PAAFs cultured as in B) and transfected with siRNAs (POU5F1/OCT4 versus si-NC control). E-F) PAAFs were transfected with miR-NC, miR-130a, tiny-LNA-NC or tiny-LNA-130 and cultivated in soft or stiff matrix. Forced miR-130a expression or stiff matrix increased collagen transcripts, LOX, and CTGF, a marker of ECM stiffening and fibrosis (E). In high ECM stiffness, tiny-LNA-130 decreased this fibrotic gene cohort (F). G] By RT-qPCR in PAAFs, constitutive PPARγ (pPPARγ) reversed the miR-130a-induced up-regulation of collagen and LOX. H) By immunoblot, miR-130a, matrix stiffening, and PPARγ knockdown decreased ApoE while inhibition of miR-130/301 (tiny-LNA-130) increased ApoE in stiff conditions. I) PAAFs exposed to exogenous ApoE were resistant to miR-130a-induced up-regulation of collagen, CTGF, and LOX. J) Luciferase reporter assays of wild type (WT) and mutated (MUT) 3′UTR sequence of LRP8 in the presence of miR-NC or miR-130a. K) By immunoblot, LRP8 was reduced by miR-130a or matrix stiffening and preserved by miR-130/301 inhibition. L) siRNA knockdown of both LRP8 and PPARγ, increased collagen and CTGF. M) Proposed model. See also FIGS. 64A-64K and FIGS. 65A-65H. In all panels, mean expression in control groups (0.2 KPa, miR-NC, Tiny-LNA-NC or si-NC in soft matrix) was assigned a fold change of 1, to which relevant samples were compared. Data are expressed as mean±SD (*P<0.05; ** P<0.01).

FIGS. 58A-58M show miR-130/301 and PAH genetic factors converge to a feedback loop that promotes ECM remodeling. A) Schema of the experimental procedure. B-C) ECM staining by Picrosirius Red revealed that miR-130a increased collagen crosslinking, while miR-130/301 inhibition (tiny-LNA-130) or ApoE reversed such effects (C). Knockdown of YAP/TAZ decreased collagen crosslinking, while knockdown PPARγ and LRP8 (siPPARγ/LRP8) increased collagen crosslinking (C). In that context, immunofluorescence for YAP (red) and nuclei (DAPI, blue) was performed on naïve PAAFs plated on ECM remodeled by the indicated conditions. Nuclear stain relative to cytosolic stain of YAP was quantified (D-E; [n=3 experiments with ten 20× fields of view analyzed per experiment]. F-G) RT-qPCR revealed that ECM remodeled by miR-130a (F) or by siPPARγ+LRP8 (G) up-regulated miR-130/301 in naïve cells plated on remodeled ECM. Conversely, ECM remodeled by tiny-LNA-130 (F) or by si-YAP/TAZ (G) down-regulated miR-130/301 in naïve cells. H) miR-130/301 induction in naïve cells cultured on matrix remodeled by miR-130a was reversed by ApoE. I-L) Factors genetically associated with PAH (ACVRL1, BMPR2, CAV1, CBLN2, ENG, KCNK3, and SMAD9) were quantified in contexts of ECM stiffening (I) and miR-130/301 manipulation (J), while miR-130/301 expression (K) and a cohort of fibrotic genes (L) were quantified after individual knockdown of these genetic factors. M) Proposed model. See also FIGS. 66A-66C. In all panels, mean expression in control groups (miR-NC, Tiny-LNA-NC, or si-NC in soft matrix) was assigned a fold change of 1, to which relevant samples were compared. Data are expressed as mean±SD (*P<0.05; ** P<0.01).

FIGS. 59A-59F show the YAP/TAZ-miR-130/301 circuit is active in rodent and human examples of PH. A-D) Monocrotaline was administered to rats (3 weeks, n=6-8/time point) to induce PH (see FIGS. 56A-56I). In serial sections of pulmonary vasculature (A), quantification of stain intensity (B) and percentage of positively stained cells (C) revealed a correlation between miR-130a, Yap1, and Oct4 expression (D). E-F) In serial sections of human pulmonary vasculature, a similar relationship was observed between increased miR-130a and YAP in PAH (controls n=8; PAH n=19). Data are expressed as mean±SD (*P<0.05; ** P<0.01).

FIGS. 60A-60F show miR-130a induces pulmonary vascular fibrosis and is reversed by the LOX inhibitor BAPN. In the presence of SU5416, wildtype mice (n=7/group) received 4 serial intrapharyngeal injections at 1 week intervals with miR-NC or miR-130a. During this period, mice were treated with 30 mg/kg/day of BAPN. BAPN lessened the miR-130/301-mediated increase in disease severity, as quantified by RVSP (A), Fulton index (RV/LV+S) (B), and pulmonary arteriolar muscularization (C). D) Picrosirius Red stain and in situ staining of mouse lung demonstrated that BAPN blunted miR-130a-mediated increases of α-smooth muscle actin (α-SMA), YAP, as well as decreased perivascular collagen crosslinking and to a lesser extent Lrp8. By RT-qPCR (E), Ctgf, collagen isoforms, and Lox were increased in diseased lung (miR-130a), but BAPN blunted these alterations. F) BAPN decreased miR-130a-induced total collagen and crosslinked collagen (i.e., increased Soluble/Insoluble collagen) in whole lung. (n=5/group). Data are expressed as mean±SEM (*P<0.05; ** P<0.01).

FIGS. 61A-61H show miR-130/301 inhibition reverses a program of fibrotic genes and ameliorates PH. A-E) Following monocrotaline exposure, rats were treated with Short-NC (n=8) or Short-130 (n=8). Short-130 delivery decreased the miR-130/301-mediated increase in PH severity, as quantified by RVSP (A) and right ventricular hypertrophy (B). C) Short-130 decreased collagen deposition and crosslinking (Picrosirius Red) as well as reversed the monocrotaline-mediated changes in α-SMA, Pparγ, Lrp8, and Yap. D) RT-qPCR demonstrated a decrease in Ctgf, collagen isoforms, and Lox, in Short-130-treated lung. E) Short-130 decreased monocrotaline-induced collagen deposition (left) and crosslinking (right) in rat lung (n=5/group). F) After two weeks of PH induction with hypoxia+SU5416, mice were serially injected with Short-NC (n=7) or Short-130 (n=7; 10 mg/kg) along with hypoxia+SU5416 for two more weeks. Short-130 treatment decreased collagen deposition and crosslinking (Picrosirius Red) as compared to control (Short-NC). G) Transcriptomic analyses of whole lung were performed from mice exposed to hypoxia+SU5416 and treated with Short-NC (n=3) or Short-130 (n=3) versus mice in normoxia+SU5416 (Control; n=3). Sylamer analyses (van Dongen et al., 2008): The x axis represents the gene list sorted according to decreasing fold change ratio (Short-130/Short-NC); the y-axis corresponds to the fold enrichment in each gene for target sequences recognized by the seed sequence of miR-130/301. Traces represent conserved (red) and non-conserved (green) sequences complementarity to the miR-130/301 seed region versus sequences complementary to a random seed region (black). Enrichment of conserved sequences (red) demonstrated a de-repression of miR-130/301 targets by Short-130 treatment. H) Genes from this transcriptomic analysis were identified according to modulation by both hypoxia and miR-130/301 inhibition. Pathway enrichment identified ECM modification as the pathway with the lowest hypergeometric p-value. Genes are color-coded according to membership in the five pathways with lowest p-values. Enlarged genes are shared by the fibrosis network (see FIG. 57A). See also FIGS. 67A-67B. Data are expressed as mean±SEM (*P<0.05; ** P<0.01).

FIGS. 62A-62H shows pharmacological inhibition of collagen crosslinking with BAPN or pharmacological activation of APOE with LXR agonist GW3965 ameliorates PH. A-D) BAPN both prevented and reversed PH severity as quantified by (A) RVSP, (B) extent of right ventricular hypertrophy (Fulton index, RV/LV+S), and (C) extent of arteriolar muscularization. D). Picrosirius Red stain and in situ staining of mouse lung demonstrated that BAPN blunted hypoxia-mediated increases of α-smooth muscle actin (α-SMA) and YAP, as well as decreased perivascular collagen crosslinking. E-H) LXR agonist GW3965 prevented PH severity as quantified by (E) RVSP, (F) extent of right ventricular hypertrophy (Fulton index, RV/LV+S), and (G) extent of arteriolar muscularization. H). Picrosirius Red stain and in situ staining of mouse lung demonstrated that GW3965 blunted hypoxia-mediated increases of •-smooth muscle actin (α-SMA) and YAP, as well as decreased perivascular collagen crosslinking. See also FIGS. 68A-68H. Data are expressed as mean±SEM (*P<0.05; ** P<0.01).

FIGS. 63A-63H show collagen crosslinking and small arterial stiffening are hallmarks of PH. A) In knockout VHL mice (VHL−/−), picrosirius Red coloration of lung tissues was imaged in parallel light to display total collagen content (top) or orthogonal light to display collagen crosslinking (bottom) (10 vessels <100 μm per mouse). B) Increased collagen concentration (Soluble) and cross-linking (Insoluble) in the lung was also confirmed by Sircol assay (n=5/group). C-F) These results was confirmed in several mouse models of PH, including, interleukin-6 transgenic mice (TgIL6; C-D) and mice infected by S. mansoni (S. mansoni) (E-F). Quantification of Picrosirius Red staining in <100 μm pulmonary vessels (10 vessels per mouse) (C and E) revealed an increase in collagen expression and collagen crosslinking under PH conditions (n=5/group). At the protein level, the soluble and insoluble (cross-linked) collagen content of the lung were assessed via Sircol assay (D and F) and confirmed the increase in collagen expression and cross-linking in diseased mice (n=5/group). G) Picrosirius Red stain of rat lung treated with the VEGF receptor antagonist SU5416 in combination with chronic hypoxia (10% O₂ for 3 weeks) as compared with normoxia+SU5416 revealed an increase in collagen expression and collagen cross-linking under disease conditions (n=5/group). H) In juvenile sheep with PAH secondary to a surgically placed pulmonary artery-aortic shunt (n=8) as compared with sham surgical controls (n=8), Sircol assays revealed an increase in collagen expression and collagen cross-linking under disease conditions. (See also FIGS. 56A-56I). Data are expressed as mean±SEM (*P<0.05; ** P<0.01).

FIGS. 64A-64K show tissue mechanics regulate the expression of miR-130/301 in a YAP-dependent and TAZ-dependent manner. Pulmonary artery smooth muscle cells (PASMCs), pulmonary artery endothelial cells (PAECs), and pulmonary artery adventitial fibroblasts (PAAF) were cultured on collagen-coated hydrogels of varying stiffness. Expression of the miR-130/301 family was induced by stiffness (A,D), and was dependent on YAP/TAZ (B,E) and POU5F1/OCT4 (C,F) in both PAECs and PASMCs. (G-H). In human PAAFs, siRNA knockdown of YAP (G) or TAZ (H) transcription factors did not reverse the ECM stiffness-mediated up-regulation of miR-130/301 family members. (I) As demonstrated by immunoblot, siRNA knockdown of YAP and TAZ was effective in human PAAFs. J-K) CTGF and POU5F1/OCT4 transcripts were quantified in PAAFs transfected with siRNAs to YAP/TAZ (J) or POU5F1/OCT4 (K). In all panels, mean expression in control groups (si-NC on soft matrix) was assigned a fold change of 1, to which relevant samples were compared. Data are expressed as mean±SD (*P<0.05; ** P<0.01).

FIGS. 65A-65H show the miR-130/301 family modulates ECM properties through the PPARγ-APOE-LRP8 regulatory axis in pulmonary arterial cells. A) Collagen, CTGF, and LOX were up-regulated by miR-130a overexpression in PAECs (left graph) and PASMCs (right graph). B) Stiffness-mediated up-regulation of collagen, CTGF, and LOX was reversed by inhibition of the miR-130/301 family (tiny-LNA-130) in PAECs (left graph) and PASMCs (right graph). C-D) In either PAECs (left graph), PASMCs (right graph), or PAAFs (D) the collagen content of conditioned media was increased by both stiffness and by miR-130a overexpression, and reversed by inhibition of the miR-130/301 family. E) In PAAFs, the collagen-content of conditioned media was increased by miR-130a and reversed by constitutive PPARγ or ApoE. F-G) In PAAFs, siRNA knockdown of PPARγ (F) or siRNA knockdown of LRP8 (G) increased collagen and LOX expression in soft matrix. H) The collagen-content of conditioned media was increased by either si-PPAR or si-LRP8 but increased to a larger extent by siPPAR+siLRP8 together. In all panels, mean expression in control groups was assigned a fold change of 1, to which relevant samples were compared. Data are expressed as mean±SD (*P<0.05; ** P<0.01).

FIGS. 66A-66C show miR-130/301-dependant ECM modification activates the feedback loop. A-B) RT-qPCR analysis of naïve cells (none transfected PAAFs) plated on ECM previously remodeled by the indicated conditions revealed that either ECM remodeled by miR-130a overexpression (A) or ECM remodeled by knockdown of PPARγ+LRP8 (B) up-regulated collagen, LOX, CTGF, and POU5F1/OCT4. Conversely, ECM remodeled by inhibition of the miR-130/301 family (tiny-LNA-130) (A) or ECM remodeled by knockdown of YAP/TAZ (B) down-regulated collagen, LOX, CTGF, and POU5F1/OCT4. C) Finally, consistent with preventing the induction of miR-130/301 family members in naïve cells cultured on matrix remodeled by miR-130a, addition of ApoE to remodeled ECM (miR-130a+ApoE cells) led to negligible alterations in transcript expression of collagen, LOX, CTGF, and POU5F1/OCT4. In all panels, mean expression in control groups was assigned a fold change of 1, to which relevant samples were compared. Data are expressed as mean±SD (*P<0.05; ** P<0.01).

FIGS. 67A-67B show short-130 inhibits the increased expression of miR-130/301 family members in lung tissue of monocrotaline-exposed rats. Male Sprague-Dawley rats (10-14 week old, N=5/group) were injected with vehicle control or 60 mg/kg monocrotaline at time 0 followed by 5 intraperitoneal injections (every 3 days) of control or miR-130/301 shortmer oligonucleotides (20 mg/kg/dose). Three days after the last injection, shortmer delivery and miRNA expression in whole lung tissue were assessed by immunochemistry (A) and by RT-qPCR (B), respectively. Data are expressed as mean±SEM (*P<0.05; ** P<0.01).

FIGS. 68A-68H show pharmacological inhibition of collagen crosslinking with BAPN or pharmacological activation of APOE with LXR agonist GW3965 ameliorates PH. A) Schema for preventing (prevention) or reducing existing hypoxic PH (rescue) with BAPN. B) In situ staining of mouse lung demonstrated that BAPN either prevented or rescued hypoxia mediated decreases of Lrp8 and Pparγ. By RT-qPCR (C), Ctgf, collagen isoforms, and Lox were increased in diseased lung (Vehicle), but BAPN blunted these alterations. D) By Sircol assay (n=5/group), whole lung collagen content (left graph) and crosslinked collagen (decreased Soluble/Insoluble collagen ratio, right graph) were nearly normalized by BAPN rescue treatment and largely maintained at baseline by BAPN prevention treatment. F) Schema for preventing hypoxic PH with GW3965. G) In situ staining of mouse lung demonstrated that GW3965 prevented hypoxia mediated decreases of Lrp8 and Pparγ. By RT-qPCR (H), Ctgf, collagen isoforms, and Lox were increased in diseased lung (Control), but Gw3965 blunted these alterations. I) By Sircol assay (n=5/group), whole lung collagen content (left graph) and crosslinked collagen (decreased Soluble/Insoluble collagen ratio, right graph) were nearly normalized by GW3965 treatment. Data are expressed as mean±SEM (*P<0.05; ** P<0.01).

FIGS. 69A-69D show network-based approaches reveal miR-130/301 fibrotic activity throughout a human diseasome. A) Strategy used to identify miRNAs that exert systems-level control over fibrosis. B) A fibrosis network, composed of known fibrotic genes (seed genes, circles) and their closest first-degree interactors (triangles). Color-coding denotes inclusion in known annotated pathways relevant to fibrosis and the extracellular matrix (from the GO, Kegg, Reactome, NCBI PID, and Biocarta databases), and demonstrates the relevance of incorporated first-degree interactors to fibrotic processes. miR-130/301 was ranked among the top five miRNAs by spanning score in this network (targets encircled in black), reflecting its robust, systems-level control over fibrosis and matrix remodeling. C) Based on transcriptomic profiling and network construction (see Methods), 137 human disease networks were grouped into cohorts, according to overlap with the fibrosis network. MiRNAs were then scored by one-way inverse correlation (one-way ANOVA) of their average assigned spanning score rank across cohorts. miR-130/301 was ranked among the top five miRNAs, underlining its importance to diseases with a strong fibrotic component (red box). D) An abbreviated human diseasome was generated from 137 disease networks (indexed in Table 14). Edges between diseases denote significant overlap (hypergeometric p-value<0.1). Diseases in red were ranked highly based on their interconnectedness with the fibrosis network and the miR-130/301 family (top 25%, as ranked in Table 14), and all were found to share a distinct cohort of fibrotic genes embedded in the overlap with the PH network. The prevalence of fibrotic diseases predicted to involve miR-130/301 provides evidence for a shared fibrotic program controlled by this miRNA family in a wide range of human pathology beyond the pulmonary vasculature (see Table 16).

FIGS. 70A-70F show positive correlation of ECM stiffening and YAP/TAZ-dependent expression of miR-130/301 in mice and humans suffering from lung fibrosis. A mouse model of lung fibrosis (bleomycin-induction, n=9-10/group) was analyzed. miR-130/301 was significantly increased by RT-qPCR (A), and serial sections of lung (B) displayed increased collagen (Picrosirius Red), miR-130a, and YAP by in situ hybridization. C) In diseased lung, miR-130a and YAP nuclear localization was positively correlated with collagen crosslinking. D) In situ staining for miR-130a and fibroblast markers (vimentin and α-SMA) was performed by fluorescent microscopy. Vimentin/miR-130a and α-SMA/miR-130a positive cells were increased in bleomycin-treated lung tissue (n=8 per groups; 5 20× fields per slide were quantified). E-F) In the lungs of patients with idiopathic pulmonary fibrosis, miR-130/301 and YAP1 were increased by RT-qPCR (E; [controls n=5, fibrosis n=15]) and in situ stain (F; [controls n=8, fibrosis n=10]). (See also FIGS. 76A-76E). Data are expressed as mean±SEM (*P<0.05; ** P<0.01).

FIGS. 71A-71E show positive correlation of ECM stiffening and YAP/TAZ-dependent expression of miR-130/301 in mice and humans suffering from liver fibrosis. A mouse model of liver fibrosis (CCl₄-induction, n=9-10/group) was analyzed. miR-130/301 was significantly increased by RT-qPCR (A), and serial sections of lung (B) displayed increased collagen (Picrosirius Red), miR-130a, and YAP by in situ hybridization. C) In diseased lung, miR-130a and YAP nuclear localization was positively correlated with collagen crosslinking. D) In situ staining for miR-130a miR-130a and stellate cell markers (desmin and α-SMA) was performed by fluorescent microscopy. Desmin/miR-130a and α-SMA/miR-130a positive cells were increased in CCl₄-treated liver (n=8 per groups; 5 20× fields per slide were quantified). E) Similarly, in situ stain demonstrated an increase in miR-130a and YAP in fibrotic liver tissue of patients (n=4 per group) with non-alcoholic steatohepatitis (NASH+fibrosis) (see also FIGS. 77A-77D). Data are expressed as mean±SEM (*P<0.05; ** P<0.01).

FIGS. 72A-72D show miR-130/301 overexpression activates ECM remodeling and liver fibrosis progression in mouse. A) As assessed by RT-qPCR, serial intraperitoneal delivery of miR-130a mimic oligonucleotide (miR-130a) increased miR-130a in whole liver in mice as compared with control (miR-NC), either with or without weekly injection of suboptimal CCl₄ dose. B) By Metavir score, either a suboptimal dose of CCl₄ or miR-130a independently increased liver fibrosis, while miR-130a+CCl₄ even more robustly increased such fibrosis. C-D) In situ hybridization of miR-130a (top row) confirmed effective delivery in the liver of mice. Immunohistochemistry also revealed that miR-130a decreased Lrp8 and Pparγ as well as slightly increased YAP nuclear localization and collagen deposition and crosslinking. Moreover, miR-130a+CCl₄ more robustly decreased Lrp8 and Pparγ as well as more robustly increased YAP nuclear localization, collagen deposition, and crosslinking.

FIGS. 73A-73F show miR-130/301 inhibition ameliorates liver fibrosis. In a model of liver fibrosis (CCl₄-induction), mice were treated with Short-NC or Short-130 (n=8/10 per group). Short-130 delivery (n=8 per group; A) and activity (n=5 per group; B) were confirmed. Short-130 decreased fibrosis, as assessed by α-SMA and collagen staining (C), as well as Metavir score (D). By in situ stain (C), Short-130 decreased vascular YAP nuclear localization and increased Pparγ and Lrp8. Short-130 also decreased transcript expression of Ctgf, collagen isoforms, and Lox (E) as well as decreased collagen deposition and collagen crosslinking (F). Data are expressed as mean±SEM (*P<0.05; ** P<0.01).

FIGS. 74A-74F show miR-130/301 inhibition ameliorates pulmonary fibrosis. In a model of lung fibrosis (bleomycin-induction), mice were treated with control (Short-NC) or miR-130/301 inhibitor (Short-130) (n=8/10 per group). Short-130 delivery (n=8 per group; A) and activity (n=5 per group; B) were confirmed. Short-130 decreased fibrosis, as assessed by α-SMA and collagen staining (C), as well as Ashcroft score (D). By in situ stain (C), Short-130 decreased vascular YAP nuclear localization and increased Pparγ and Lrp8. Short-130 also decreased transcript expression of Ctgf, collagen isoforms, and Lox (E) as well as decreased collagen deposition and collagen crosslinking (F). Data are expressed as mean±SEM (*P<0.05; ** P<0.01).

FIGS. 75A-75D show pharmacological activation of APOE with LXR agonist GW3965 ameliorates pulmonary fibrosis. In a model of lung fibrosis (bleomycin exposure), mice were treated with the LXR agonist GW3965 or control (n=6 per group) via dietary intake (100 mg/kg). GW3965 ameliorated the degree of lung fibrosis as quantified by (B) Aschcroft score and α-SMA labeling (A). Picrosirius Red stain and in situ staining of mouse lung (A) demonstrated that GW3965 blunted bleomycin-mediated increases of collagen crosslinking and YAP, as well as decreased of Lrp8 and Pparγ expression. C) By RT-qPCR, Ctgf, collagen isoforms, and Lox were increased in diseased lung (bleomycin), but GW3965 reduced such increased expression. D) Unifying model of the central role of the YAP/TAZ-miR-130/301 circuit in control of extracellular matrix plasticity across related diseases. Data are expressed as mean±SEM (*P<0.05; ** P<0.01).

FIGS. 76A-76E show a positive correlation exists between miR-130a expression, collagen crosslinking, and Yap1 activation in a mouse model of bleomycin-induced lung fibrosis and in lung fibrosis patients. A) In bleomycin-induced lung fibrosis in mice, expression levels of α-SMA, Pparγ, and Lrp8 were assessed by immunohistochemistry. Twenty-one days (n=10) after bleomycin injection, levels of Pparγ and Lrp8 were significantly decreased by bleomycin as compared with PBS (n=9). B) RT-qPCR confirmed an increase in collagen and the collagen cross-linking gene Lox in diseased lung. C) Serial lung sections derived from mice treated with bleomycin were stained for collagen (Picrosirius Red), miR-130a, and Yap1 (see also FIG. 2). In this panel (C), a higher magnification is presented. D-E) Serial lung sections from patients suffering from idiopathic pulmonary fibrosis were stained for collagen (Picrosirius Red), Yap1, and miR-130a (D). Quantification of miR-130a stain intensity and percentage of positively stained parenchymal cells revealed a correlation among miR-130a, Yap1 nuclear localization, and collagen crosslinking (E). Data are expressed as mean±SEM (*P<0.05; ** P<0.01).

FIGS. 77A-77D show a positive correlation exists between miR-130a expression, collagen crosslinking, and Yap1 activation in a mouse model of CCl₄-induced liver fibrosis and in liver fibrosis patients. A) In CCl₄-induced liver fibrosis in mice, expression levels of α-SMA, Pparγ, and Lrp8 were assessed by immunohistochemistry. Four weeks (n=10) or six weeks (n=9) after CCl₄ exposure, levels of Pparγ and Lrp8 were significantly decreased as compared with vehicle control (n=9, Oil). B) RT-qPCR confirmed an increase in collagen and the collagen cross-linking gene Lox in diseased liver. C-D) Serial hepatic sections derived from patients suffering from liver disease with varying levels of fibrosis were stained for YAP1 and miR-130a (C). Quantification of miR-130a stain intensity and percentage of YAP1 nuclear positive cells revealed a correlation among miR-130a and Yap1 nuclear localization (D). Data are expressed as mean±SEM (*P<0.05; ** P<0.01).

### DETAILED DESCRIPTION

Aspects disclosed herein are based, in part, on inventors' discovery that levels of multiple members of the miR-130/301 family are elevated in subject having pulmonary hypertension or pulmonary hypertension pulmonary arterial hypertension, and inhibition of multiple family members simultaneously has the most robust response in disease amelioration, as compared with single miRNA inhibition alone.

Accordingly, the disclosure provides a method of inhibiting, preventing or treating pulmonary hypertension (PH) or pulmonary artiral hypertension (PAH) or a symptom thereof in a subject. Generally the method comprises inhibiting activity or function of at least one member (e.g., one, two, three, four, five or more members) of microRNA-130/301 family in the subject.

Without limitations, the method disclosed herein can be performed for any type of pulmonary hypertension such as revised in the World Health Organisation Classification of pulmonary hypertension and selected from the group consisting of Pulmonary arterial hypertension that develops as sporadic disease (idiopathic), as an inherited disorder (familial), or in association with certain conditions (collagen vascular diseases, congenital systemic-to-pulmonary shunts (large, small, repaired, or nonrepaired), portal hypertension, human immunodeficiency virus (HIV) infection, ingestion of drugs or dietary products and toxins (anorexigens, rapeseed oil, L-tryptophan, methamphetamine, and cocaine), or in association with other conditions (thyroid disorders, glycogen storage disease, Gaucher disease, hereditary hemorrhagic telangiectasia, hemoglobinopathies, myeloproliferative disorders, and splenectomy)), or associated with significant venous or capillary involvement (pulmonary veno—occlusive disease and pulmonary capillary hemangiomatosis) and persistent pulmonary hypertension of the newborn; Pulmonary venous hypertension (left-sided atrial or ventricular heart disease, left-sided valvular heart disease); Pulmonary hypertension associated with hypoxemia (chronic obstructive pulmonary disease, Interstitial lung disease; Sleep-disordered breathing, alveolar hypoventilation disorders, Long-term exposure to high altitudes, developmental abnormalities); Pulmonary hypertension due to chronic thrombotic or embolic disease (thromboembolic obstruction of proximal pulmonary arteries, thromboembolic obstruction of distal pulmonary arteries, Pulmonary embolism (tumor, parasites, foreign material)); Miscellaneous (sarcoidosis, pulmonary Langerhans′-cell histiocytosis, lymphangiomatosis, compression of pulmonary vessels (adenopathy, tumor, fibrosing mediastinitis)

The inventors have also discovered that miR-130/301 family also targets fibrotic diseases. Accordingly, the disclosure also provides a method of inhibiting, preventing or treating pulmonary fibrotic or or fibroproliferative disease or a symptom thereof in a subject, wherein the method comprises by comprises inhibiting activity or function of at least one member (e.g., one, two, three, four, five or more members) of microRNA-130/301 family in the subject.

The inventors have further discovered that miR-130/301 family can also modulate extracellular matrix deposition and vascular/tissue stiffness as well. Thus the disclosure also provides a method of modulating extracellular matrix deposition or vascular/tissue stiffness in a subject in need thereof. The method comprising inhibiting activity or function of at least one member (e.g., one, two, three, four, five or more members) of microRNA-130/301 family in the subject. In some embodiments, modulating extracellular matrix deposition or vascular/tissue stiffness means inhibiting or reducing extracellular matrix deposition or vascular/tissue stiffness.

Extracellular matrix deposition is thought to play roles in cell adhesion, cell signaling, bone mineralization, inflammatory responses, regulation of embryogenesis, regulation of tissue differentiation and/or maturation (e.g. airway branching in lung development), tissue degradation, and establishing cell polarity. Dynamic remodeling of the extracellular matrix (ECM) is essential for development, wound healing and normal organ homeostasis. Life-threatening pathological conditions arise when ECM remodeling becomes excessive or uncontrolled. Thus, the methods disclosed herein for modulating extracellular matrix deposition can also be used for diagnosis and/or treatment cancer/metastasis.

As used herein, the term “activity” or “function” in reference to microRNA refers to binding (or hybridizing) to a polynuclotide (e.g., mRNA) which comprises a nucleotide sequence which is substantially complementary to the miRNA seed sequence. In context of miR-130/301, activity means binding to a polynuclotide (e.g., mRNA) which comprises a nucleotide sequence which is substantially complementary to 5′-AGUGCAA-3′ (SEQ ID NO: 1). In some embodiments, in context of miR-130/301, activity means binding to a polynuclotide (e.g., mRNA) which comprises a nucleotide sequence 5′-UUGCAC-3′ or 5′-UUGCACUA-3′. In some emodiments, in context of miR-130/301, activity means binding to a polynuclotide (e.g., mRNA) which comprises a nucleotide sequence which is substantially complementary to the miRNA-130/301 family seed sequence, i.e., comprises nucleotide sequence which is fully complementary to the miRNA-130/301 family seed sequence or can have one, two, three, four, five or more mismatches to the miRNA-130/301 family seed sequence.

In some emodiments, in context of miR-130/301, activity means binding to a polynuclotide (e.g., mRNA) which comprises a nucleotide sequence which is substantially complementary to the miRNA-130/301 family seed sequence, i.e., comprises nucleotide sequence which is fully complementary to the miRNA-130/301 family seed sequence or can have one, two, three, four, five or more mismatches to the miRNA-130/301 family seed sequence.

As used herein, the phrase “inhibiting the activity” in reference to a miR-130/301 family member includes inhibiting or reducing the expression of the miR-130/301 family member.

In the various embodiments of the methods disclosed herein, the method comprises administering to the subject an effective amount of an agent that inhibits the activity of at least one member (e.g., one, two, three, four, five or more members) of microRNA-130/301 family. Without limitations the miR-130/301 family member can be selected from the group consisting of miR-130a, miR-130b, miR-301a, miR-301b, and any combinations thereof. In some embodiments, the miR-130/301 family member includes miR-454, which has the same seed sequence.

The inventros have discovered inter alia that inhibition of multiple family members simultaneously can provide most robust response in disease amelioration, as compared with single miRNA inhibition alone. Thus, in various embodiments, the method comprises inhibiting the activity of at least two of, at least three of, at least four or at least five of miR-130a, miR-130b, miR-301a, miR-301b, and miR-454. In some embodiments, the method comprises inhibiting the activity of at least two members of miR-130/301 family, for example, method comprises inhibiting the activity of at least miR-130a and miR-130b; at least miR-130a and miR-301a; at least miR-130a and miR-301b; at least miR-130b and miR-301a; at least miR-130b and miR-301b; at least miR-301a and miR-301b; at least miR-130a and miR-454; at least miR-130b and miR-454; at least miR-301a and miR-454; or at least miR-301b and miR-454. In some embodiments, the method comprises inhibiting the activity of at least three members of miR-130/301 family, for example, method comprises inhibiting the activity of at least miR-130a, miR-130b and miR-301a; at least miR-130a, miR-130b and miR-301b; at least miR-130b, miR-301a and miR-301b; at least miR-130a, miR-130b and miR-454; at least miR-130a, miR-301a and miR-454; at least miR-130a, miR-301b and miR-454; at least miR-130b, miR-301a and miR-454; at least miR-130b, miR-301b and miR-454; or at least miR-301a, miR-301b and miR-454. In some embodiments, the method comprises inhibiting the activity of four members of the miR-130/301 family, for example, method comprises inhibiting the activity of miR-130a, miR-130b, miR-301a and miR-301b; .miR-130a, miR-130b, miR-301a and miR-454; miR-130a, miR-130b, miR-301b and miR-454; miR-130a, miR-301a, miR-301b and miR-454; or miR-130b, miR-301a, miR-301b and miR-454.

The agent that inhibits miR-130/301 is also referred to as an anti-miR, e.g., anti-miR-130/301 agent, herein. The terms “anti-miR,” “anti-miR agent,” “antimir” “microRNA inhibitor” or “miR inhibitor” is synonymous and refers to any agent which interferes with the function of the target miRNA. Without limitations, the agent for inhibiting the activity of miR-130/301 can be any molecule, compound or composition that inhibits, reduces or interferes with binding of the miR-130/301 family member to its target mRNA. Alternatively, or in addition, the agent can be any molecule, compound or composition that increases the level of miRNA-130/301 targeted mRNA or the product encoded therein. The agent can also be a molecule, compound or composition which decreases, inhibits, or silences the expression of the miR-130/301.

In some embodiments, the anti-miR-130/301 agent inhibits the activity of at least two of, at least three of, at least four or at least five of miR-130a, miR-130b, miR-301a, miR-301b, and miR-454. Accordingly, in some embodiments, the anti-miR-130/301 agent inhibits the activity of at least two members of miR-130/301 family, for example, the anti-miR-130/301 agent inhibits the activity of at least miR-130a and miR-130b; at least miR-130a and miR-301a; at least miR-130a and miR-301b; at least miR-130b and miR-301a; at least miR-130b and miR-301b; at least miR-301a and miR-301b; at least miR-130a and miR-454; at least miR-130b and miR-454; at least miR-301a and miR-454; or at least miR-301b and miR-454. In some embodiments, the anti-miR-130/301 agent inhibits the activity of at least three members of miR-130/301 family, for example, anti-miR-130/301 agent inhibits the activity of at least miR-130a, miR-130b and miR-301a; at least miR-130a, miR-130b and miR-301b; at least miR-130b, miR-301a and miR-301b; at least miR-130a, miR-130b and miR-454; at least miR-130a, miR-301a and miR-454; at least miR-130a, miR-301b and miR-454; at least miR-130b, miR-301a and miR-454; at least miR-130b, miR-301b and miR-454; or at least miR-301a, miR-301b and miR-454. In some embodiments, the anti-miR-130/301 agent inhibits the activity of four members of the miR-130/301 family, for example, anti-miR-130/301 agent inhibits activity of miR-130a, miR-130b, miR-301a and miR-301b; .miR-130a, miR-130b, miR-301a and miR-454; miR-130a, miR-130b, miR-301b and miR-454; miR-130a, miR-301a, miR-301b and miR-454; or miR-130b, miR-301a, miR-301b and miR-454.

The terms “decrease”, “inhibit”, and “silence” in context of expression of a target (e.g., miR-130/301) refer to the at least partial suppression of the expression of the gene encoding the target whose expression is to inhibited as manifested by a reduction of the amount of mRNA transcribed from gene which may be isolated from a first cell or group of cells in which the gene is transcribed and which has or have been treated such that the expression of the gene is inhibited, as compared to a second cell or group of cells substantially identical to the first cell or group of cells but which has or have not been so treated (control cells).

Alternatively, the degree of inhibition can be given in terms of a reduction of a parameter that is functionally linked to gene transcription, e.g. the amount of protein encoded by the gene which is secreted by a cell, or the number of cells displaying a certain phenotype, e.g apoptosis. In principle, gene silencing can be determined in any cell expressing the target, either constitutively or by genomic engineering, and by any appropriate assay. For example, in certain instances, expression of the gene is suppressed by at least about 20%, 25%, 35%, or 50% by administration of the anti-miR-130/301 agent. In some embodiment, the gene is suppressed by at least about 60%, 70%, or 80% by administration of the anti-miR-130/301 agent. In some embodiments, the gene is suppressed by at least about 85%, 90%, or 95% by administration of the anti-miR-130/301 agent.

Exemplary agents for inhibiting the activity of miR-130/301 can be, for example, a small molecule, nucleic acid, nucleic acid analogue, peptide, protein, antibody, or variants and fragments thereof. In some embodiments, the nucleic acid agent can be DNA, RNA, nucleic acid analogue, peptide nucleic acid (PNA), pseudo-complementary PNA (pcPNA), locked nucleic acid (LNA) or analogue thereof.

In some embodiments, the agent that inhibits the activity of a miR-130/301 family member is a nucleic acid, for example, an oligonucleotide. Exemplary nucleic acids useful as inhibitors of miR-130/301 include, but are not limited to, small inhibitory RNA (RNAi), siRNA, antisense oligonucleotides, microRNA, shRNA, miRNA, and analogues and homologues and variants thereof effective in gene silencing.

In some embodiments, an anti-miR is an oligonucleotide or modified oligonucleotide that interferes with the activity of a specific miRNA, e.g., at least one member (e.g., one, two, three, font, five or more members) of miR-130/301 family. In some embodiments, where an anti-miR is an oligonucleotide, an anti-miR can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the pre-miRNA or mature strand (or strands) of the miRNA to be targeted. In addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences. In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands. Furthermore, microRNA inhibitors can be linked to conjugate moieties in order to facilitate uptake of the inhibitor into a cell.

An anti-miR agent encompassed for use in the methods, compositions and kits as disclosed herein is a nucleic acid, or analogue or mimetic thereof which comprises a nucleotide sequence that is substantially complementary to at least a portion of the miR-130a, miR-103b, miR-301a, and/or miR-301b nucleotide sequence. In some embodiments, the anti-miR-130/301 agent is an oligonucleotide, wherein the oligonucleotide comprises a sequence that is substantially complementary to at least a portion of sequence 5′-AGUGCAA-3′ (SEQ ID NO: 1). For example, the oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, or 8) consecutive nucleotides of sequence 5′-AGUGCAA-3′ (SEQ ID NO: 1).

In some embodiments, the anti-miR-130/301 agent is an oligonucleotide, wherein the oligonucleotide comprises a sequence that is substantially complementary to at least a portion of sequence selected from:

(human miR-130a)
(SEQ ID NO: 20)
UGCUGCUGGCCAGAGCUCUUUUCACAUUGUGCUACUGUCUGCACCUGUCA
CUAGCAGUGCAAUGUUAAAAGGGCAUUGGCCGUGUAGUG;
(human miR-130b)
(SEQ ID NO: 21)
GGCCUGCCCGACACUCUUUCCCUGUUGCACUACUAUAGGCCGCUGGGAAG
CAGUGCAAUGAUGAAAGGGCAUCGGUCAGGUC;
(human miR-301a)
(SEQ ID NO: 22)
ACUGCUAACGAAUGCUCUGACUUUAUUGCACUACUGUACUUUACAGCUAG
CAGUGCAAUAGUAUUGUCAAAGCAUCUGAAAGCAGG;
(human miR-301b)
(SEQ ID NO: 23)
GCCGCAGGUGCUCUGACGAGGUUGCACUACUGUGCUCUGAGAAGCAGUGC
AAUGAUAUUGUCAAAGCAUCUGGGACCA;
(mouse miR-130a)
(SEQ ID NO: 24)
GAGCUCUUUUCACAUUGUGCUACUGUCUAACGUGUACCGAGCAGUGCAAU
GUUAAAAGGGCAUC;
and
(mouse miR-130b)
(SEQ ID NO: 25)
CAGUGGGCUUGUUGGACACUCUUUCCCUGUUGCACUACUGUGGGCCUCUG
GGAAGCAAUGAUGAAAGGGCAUCUGUCGGGCC.

For example, the oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of a sequence selected from: human miR-130a (SEQ ID NO: 20); human miR-130b (SEQ ID NO: 21); human miR-301a (SEQ ID NO: 22); human miR-301b (SEQ ID NO: 23); mouse miR-130a (SEQ ID NO: 24) and mouse miR-130b (SEQ ID NO: 25).

In some embodiments, the anti-miR-130/301 agent is an oligonucleotide, wherein the oligonucleotide comprises a sequence that is substantially complementary to at least a portion of sequence selected from: has-miR-130a-3p (cagugcaauguuaaaagggcau) (SEQ ID NO: 4); has-miR-130b-3p (cagugcaaugaugaaagggcau) (SEQ ID NO: 5); has-miR-301a-3p (cagugcaauaguauugucaaagc) (SEQ ID NO: 6); has-miR-301b-3p (cagugcaaugauauugucaaagc) (SEQ ID NO: 13) and has-miR-454-3p (uagugcaauauugcuuauagggu) (SEQ ID NO: 26).

For example, the oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of a sequence selected from: has-miR-130a-3p (SEQ ID NO: 4); has-miR-130b-3p (SEQ ID NO: 5); has-miR-301a-3p (SEQ ID NO: 6); has-miR-301b-3p (SEQ ID NO: 6) and has-miR-454-3p (SEQ ID NO: 26).

In some embodiments, the agent is an oligonucleotide, wherein the oligonucleotide comprises the sequence 5′-TTGCACT-3′ (SEQ ID NO: 2) or 5′-ATTGCACT-3′ (SEQ ID NO: 3).

As discussed herein, encompassed for use in the methods, compositions and kits herein are anti-miR-130/301 agents which can be any or a combination of RNA interference molecules, for example anti-miRs, oligonucleotides, LNA, miRNA, siRNA, shRNA, or proteins, small molecules, nucleic acids, nucleic acid analogues, aptamers, antibodies, peptides and variants and analogues thereof. In some embodiments, where the anti-miR-130/301 agent is an antibody, the antibody can be a recombinant antibody, humanized antibody, chimeric antibody, modified antibody, monoclonal antibody, polyclonal antibody, miniantibody, dimeric miniantibody, minibody, diabody or tribody or antigen-binding variants, analogues or modified versions thereof.

In some embodiments, the anti-miR-130/301 agent is an antisense oligonucleotide. One of skill in the art is well aware that single-stranded oligonucleotides can hybridize to a complementary target sequence and prevent access of the translation machinery to the target RNA transcript, thereby preventing protein synthesis. The single-stranded oligonucleotide can also hybridize to a complementary RNA and the RNA target can be subsequently cleaved by an enzyme such as RNase H and thus preventing translation of target RNA. Alternatively, or in addition to, the single-stranded oligonucleotide can modulate the expression of a target sequence via RISC mediated cleavage of the target sequence, i.e., the single-stranded oligonucleotide acts as a single-stranded RNAi agent. A “single-stranded RNAi agent” as used herein, is an RNAi agent which is made up of a single molecule. A single-stranded RNAi agent can include a duplexed region, formed by intra-strand pairing, e.g., it can be, or include, a hairpin or pan-handle structure.

In some embodiments, the anti-miR-130/301 agent is RNA-interference or RNA interference molecule, including, but not limited to double-stranded RNA, such as siRNA, double-stranded DNA or single-stranded DNA. In some embodiments, an anti-miR-130/301 agent is a single-stranded RNA (ssRNA), a form of RNA endogenously found in eukaryotic cells as the product of DNA transcription. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

Numerous specific siRNA molecules have been designed that have been shown to inhibit gene expression (Ratcliff et al. Science 276:1558-1560, 1997; Waterhouse et al. Nature 411:834-842, 2001). In addition, specific siRNA molecules have been shown to inhibit, for example, HIV-1 entry to a cell by targeting the host CD4 protein expression in target cells thereby reducing the entry sites for HIV-1 which targets cells expressing CD4 (Novina et al. Nature Medicine, 8:681-686, 2002). Short interfering RNA have further been designed and successfully used to silence expression of Fas to reduce Fas-mediated apoptosis in vivo (Song et al. Nature Medicine 9:347-351, 2003).

It has been shown in plants that longer, about 24-26 nt siRNA, correlates with systemic silencing and methylation of homologous DNA. Conversely, the about 21-22 nt short siRNA class correlates with mRNA degradation but not with systemic signaling or methylation (Hamilton et al. EMBO J. 2002 Sep. 2; 21(17):4671-9). These findings reveal an unexpected level of complexity in the RNA silencing pathway in plants that may also apply in animals. In higher order eukaryotes, DNA is methylated at cytosines located 5′ to guanosine in the CpG dinucleotide. This modification has important regulatory effects on gene expression, especially when involving CpG-rich areas known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosomes of females. Aberrant methylation of normally unmethylated CpG islands has been documented as a relatively frequent event in immortalized and transformed cells and has been associated with transcriptional inactivation of defined tumor suppressor genes in human cancers. In this last situation, promoter region hypermethylation stands as an alternative to coding region mutations in eliminating tumor suppression gene function (Herman, et al.). The use of siRNA molecules for directing methylation of a target gene is described in U.S. Provisional Application No. 60/447,013, filed Feb. 13, 2003, referred to in U.S. Patent Application Publication No. 20040091918.

It is also known that the RNA interference does not have to match perfectly to its target sequence. Preferably, however, the 5′ and middle part of the antisense (guide) strand of the siRNA is perfectly complementary to the target nucleic acid sequence.

The RNA interference-inducing molecule functioning as anti-miR-130/301 agent includes RNA molecules that have natural or modified nucleotides, natural ribose sugars or modified sugars and natural or modified phosphate backbone. Accordingly, the RNA interference-inducing molecules functioning as anti-miR-130/301 agen includes, but are not limited to, unmodified and modified double stranded (ds) RNA molecules including short-temporal RNA (stRNA), small interfering RNA (siRNA), short-hairpin RNA (shRNA), microRNA (miRNA), and double-stranded RNA (dsRNA), (see, e.g. Baulcombe, Science 297:2002-2003, 2002). The dsRNA molecules, e.g. siRNA, also may contain 3′ overhangs, preferably 3′UU or 3′TT overhangs. In one embodiment, the siRNA molecules do not include RNA molecules that comprise ssRNA greater than about 30-40 bases, about 40-50 bases, about 50 bases or more. In one embodiment, the siRNA molecules have a double stranded structure. In one embodiment, the siRNA molecules are double stranded for more than about 25%, more than about 50%, more than about 60%, more than about 70%, more than about 80% or more than about 90% of their length.

Anti-miRs, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the compositions and methods disclosed herein. Anti-miRs can be used to efficiently silence endogenous miRNAs by forming duplexes comprising the anti-miR and endogenous miRNA, thereby preventing miRNA-induced gene silencing.

In some embodiments, the anti-miR-130/301 agent is an antagomir. Antagomirs are oligonucleotide anti-miRs that harbor various modifications for RNAse protection and pharmacologic properties, such as enhanced tissue and cellular uptake. They differ from normal RNA by, for example, complete 2′-O-methylation of sugar, phosphorothioate intersugar linkage and, for example, a cholesterol-moiety at 3′-end. In some embodiments, antagomir comprises a 2′-O-methylmodification at all nucleotides, a cholesterol moiety at 3′-end, two phosphorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

In some embodiments, the anti-miR-130/301 agent is ribozyme. In some embodiments, the the anti-miR-130/301 agent is ribozyme that cleaves the target microRNA. Ribozymes are oligonucleotides having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). At least six basic varieties of naturally-occurring enzymatic RNAs are known presently. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Methods of producing a ribozyme targeted to any target sequence are known in the art. Ribozymes can be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to up-regulate genes that are suppressed by the binding of a transcription factor. Examples of the utilization of decoy oligonucleotides can be found in Mann et al., J. Clin. Invest., 2000, 106: 1071-1075, which is expressly incorporated by reference herein, in its entirety. Thus, in some embodiments, the anti-miR-130/301 agent is a decoy oligonucleotide.

The inventors have demonstrated that the miR-130/301 family can modulate the activity and/or expression level of a number of different targets. Without wishing to be bound by a theory, miR-130/301 can modulate the activity or expression of proliferator-activated receptor gamma (PPARγ), apelin (APLN), nitric oxide synthase(NOS3), TIMP2, miR-424, miR-503, miR-204, COL1A1, COL3A1, CTGF, LOX, FGF2, STAT3, phosphorylated STAT3, vascular growth factor-A (VEGFA), endothelin-1 (EDN1, MMP2, APOE, LRP8, or a nucleic acid encoding the same. For example, the inventors have shown that increased levels of miR-130/301 family decrease the level of peroxisome proliferator-activated receptor gamma (PPARγ), apelin, nitric oxide synthase (NOS3), TIMP2, miR-424, miR-503, and miR-204, or a nucleic acid encoding the same relative to a control or reference level. The inventors have also shown that increased levels of miR-130/301 family increase the level of FGF2, STAT3, phosphorylated STAT3, vascular growth factor-A (VEGFA), endothelin-1 (EDN1), COL1A1, COL3A1, CTGF, LOX, and MMP2, or a nucleic acid encoding the same relative to a control or reference level.

Thus, inhibiting the activity of miR-130/301 family can increase the activity or expression of PPARγ, TIMP2, miR-204, miR-424, miR-503, LRP8, NOS3, Apelin, or a nucleic acid encoding same. Alternatively, or in addition, inhibiting the activity of miR-130/301 family can decrease the activity or expression of one or more of MMP2, EDN1, COL1A1, COL3A1, CTGF, LOX, VEGFA, FGF2, STAT3, Phospho-STAT3, or a nucleic acid encoding the same. Accordingly, in some embodiments, inhibiting the activity of miR-130/301 increases the activity or expression of one or more of PPARγ, apelin, nitric oxide synthase (NOS3), TIMP2, miR-424, miR-503, miR-204, LRP8, or a nucleic acid encoding the same. In some embodiments, inhibiting the activity of miR-130/301 decreases the activity or expression of one or more of MMP2, EDN1, COL1A1, COL3A1, CTGF, LOX, VEGFA, FGF2, STAT3, Phospho-STAT3, or a nucleic acid encoding the same.

The inventors have also discovered that the miR-130/301 family modulates PPARγ with distinct cell type-specific consequences. For example, to promote proliferation, miR-130/301 modulates apeline-miR424/503-FGF2 signaling in pulmonary arterial endothelial cells and separately modulates STAT-3-miR-204 signaling in pulmonary arterial smooth muscle cells. Thus, the anti-miR-130/301 agent can modulate the activity or expression of a miR-130/301 target in cell type-specific manner. For example, the anti-miR-130/301 agent increases apelin, miR-424, miR-503, or a nucleic acid encoding the same in a pulmonary arterial endothelial cell (PAEC). In contrast, the inhibitor increases miR-204 or a nucleic acid encoding the same in a pulmonary arterial smooth muscle cell (PASMC). The anti-miR-130/301 agent decreases FGF2 or the nucleic acid encoding FGF2 in PAEC, but decreases STAT3 or the nucleic acid encoding STAT3 in a PASMC.

The inventors have further shown that miR-130/301 family expression is increased by matrix stiffness in a YAP/TAZ- and OCT4-dependent fashion. Thus, in some embodiments, the anti-miR-130/301 agent inhibits the activity or expression of YAP/TAZ or OCT4.

In another aspect, the disclosure provides an isolated or synthetic oligonucleotide which can be used as an inhibitor of miR-130/301, i.e., an-anti-miR-130/301 agent. The oligonucleotide can hybridize to a complementary RNA, e.g., mRNA, pre-mRNA, microRNA, or pre-microRNA and reduce the activity, expression, or amount of the complementary RNA, e.g., target RNA. This can be by reducing access of the translation machinery to the target RNA transcript, thereby preventing protein synthesis. The oligonucleotide can induce cleavage of the complementary RNA by an enzyme, such RISC mediated cleavage or RNase H and thus reducing the amount of the target RNA. The oligonucleotide itself can cleave the complementary RNA, e.g., a ribozyme, RISC mediated cleavage or RNase H and thus reducing the amount of the target RNA. The oligonucleotide, by hybridizing to the target RNA, can inhibit binding of the target RNA to another complementary strand.

In some embodiments, the oligonucleotide comprises a sequence that is substantially complementary to at least a portion of sequence 5′-AGUGCAA-3′ (SEQ ID NO: 1). For example, the oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, or 8) consecutive nucleotides of sequence: 5′-AGUGCAA-3′ (SEQ ID NO: 1).

In some embodiments, the oligonucleotide comprises a sequence that is substantially complementary to at least a portion of sequence selected from: human miR-130a (SEQ ID NO: 20); human miR-130b (SEQ ID NO: 21); human miR-301a (SEQ ID NO: 22); human miR-301b (SEQ ID NO: 23); mouse miR-130a (SEQ ID NO: 24); mouse miR-130b (SEQ ID NO: 25); and UCUGUUUAUCACCAGAUCCUAGAACCCUAUCAAUAUUGUCUCUGCUGUGUAAAUA GUUCUGAGUAGUGCAAUAUUGCUUAUAGGGUUUUGGUGUUUGGAAAGAACAAUG GGCAGG (miR-454) (SEQ ID NO: 27).

For example, the oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of a sequence selected from: human miR-130a (SEQ ID NO: 20); human miR-130b (SEQ ID NO: 21); human miR-301a (SEQ ID NO: 22); human miR-301b (SEQ ID NO: 23); mouse miR-130a (SEQ ID NO: 24); mouse miR-130b (SEQ ID NO: 25); and miR-454 (SEQ ID NO: 27).

In some embodiments, the oligonucleotide comprises a sequence that is substantially complementary to at least a portion of sequence selected from the group consisting of has-miR-130a-3p (SEQ ID NO: 4); has-miR-130b-3p (SEQ ID NO: 5); has-miR-301a-3p (SEQ ID NO: 6); has-miR-301b-3p (SEQ ID NO: 7) and has-miR-454-3p (SEQ ID NO: 26). For example, the oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of a sequence selected from the group consisting of has-miR-130a-3p (SEQ ID NO: 4); has-miR-130b-3p (SEQ ID NO: 5); has-miR-301a-3p (SEQ ID NO: 6); has-miR-301b-3p (SEQ ID NO: 7) and has-miR-454-3p (SEQ ID NO: 26). In some embodiments, the oligonucleotide comprises the sequence 5′-TTGCACT-3′ (SEQ ID NO: 2) or 5′-ATTGCACT-3′ (SEQ ID NO: 3).

### Oligonucleotides

As used herein, the term “nucleic acid” or “oligonucleotide” or “polynucleotide” refers to a polymer or an oligomer of nucleotide or nucleoside monomers consisting of nucleobases, sugars and intersugar linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

The oligonucleotide can be single-stranded or double-stranded. A single-stranded oligonucleotide can have double-stranded regions and a double-stranded oligonucleotide can have single-stranded regions. The oligonucleotide can have a hairpin structure or have a dumbbell structure. The oligonucleotide can be circular, e.g., wherein the 5′end of the oligonucleotide is linked to the 3′ end of the oligonucleotide. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe that can hybridize to the target sequence under stringent hybridization conditions. Thus, a nucleic acid also encompasses a probe that hybridizes under stringent hybridization conditions.

The oligonucleotides described herein can comprise any oligonucleotide modification described herein and below. In some embodiments, the oligonucleotide comprises at least one modification. In some embodiments, the modification is selected from the group consisting of a sugar modification, a non-phosphodiester inter-sugar (or inter-nucleoside) linkage, nucleobase modification, and ligand conjugation.

In some embodiments, the oligonucleotide comprises at least two different modifications selected from the group consisting of a sugar modification, a non-phosphodiester inter-sugar linkage, nucleobase modification, and ligand conjugation. In some embodiments, the at least two different modifications are present in the same subunit of the oligonucleotide, e.g. present in the same nucleotide.

As used herein, an oligonucleotide can be of any length. In some embodiments, oligonucleotides can range from about 6 to 100 nucleotides in length. In various related embodiments, the oligonucleotide can range in length from about 10 to about 50 nucleotides, from about 10 to about 35 nucleotides, from about 15 to about 30 nucleotides, from about 20 to about 30 nucleotides in length. In some embodiments, oligonucleotide is from about 8 to about 39 nucleotides in length. In some embodiments, the oligonucleotide is 6 to 25 nucleotides in length (e.g., 6, 7, 8, 9, 10, 11, 12, 13, 14, 15, 16, 18, 19, 20, 21, 22, 23, or 24 nucleotides in length). In some embodiments the oligonucleotide is 25-30 nucleotides. In some embodiments, the single-stranded oligonucleotide is 15 to 29 nucleotides in length. In some other embodiments, the oligonucleotide is from about 18 to about 25 nucleotides in length. In some embodiments, the oligonucleotide is about 23 nucleotides in length. In some embodiments, a single-stranded oligonucleotide is 6, 7, 8, 9, 10, 11, 12, 13, 14, 15, 16 17, 18, or 19 nucleotides in length.

The oligonucleotide can be completely DNA, completely RNA, or comprise both RNA and DNA nucleotides. It is to be understood that when the oligonucleotide is completely DNA, RNA, or a mix of both, the oligonucleotide can comprise one or more oligonucleotide modifications described herein.

An oligonucleotide can be a chimeric oligonucleotide. As used herein, a “chimeric” oligonucleotide” or “chimera” refers to an oligonucleotide which contains two or more chemically distinct regions, each made up of at least one monomer unit, i.e., a modified or unmodified nucleotide in the case of an oligonucleotide. Chimeric oligonucleotides can be described as having a particular motif. In some embodiments, the motifs include, but are not limited to, an alternating motif, a gapped motif, a hemi-mer motif, a uniformly fully modified motif and a positionally modified motif. As used herein, the phrase “chemically distinct region” refers to an oligonucleotide region which is different from other regions by having a modification that is not present elsewhere in the oligonucleotide or by not having a modification that is present elsewhere in the oligonucleotide. An oligonucleotide can comprise two or more chemically distinct regions. As used herein, a region that comprises no modifications is also considered chemically distinct.

A chemically distinct region can be repeated within an oligonucleotide. Thus, a pattern of chemically distinct regions in an oligonucleotide can be realized such that a first chemically distinct region is followed by one or more second chemically distinct regions. This sequence of chemically distinct regions can be repeated one or more times. Preferably, the sequence is repeated more than one time. Both strands of a double-stranded oligonucleotides can comprise these sequences. Each chemically distinct region can actually comprise as little as a single nucleotide. In some embodiments, each chemically distinct region comprises 1, 2, 3, 4, 5, 6, 7, 8, 9, 10, 11, 12, 13, 14, 15, 16, 17 or 18 nucleotides.

In some embodiments, alternating nucleotides comprise the same modification, e.g. all the odd number nucleotides in a strand have the same modification and/or all the even number nucleotides in a strand have the similar modification to the first strand. In some embodiments, all the odd number nucleotides in an oligonucleotide have the same modification and all the even numbered nucleotides have a modification that is not present in the odd number nucleotides and vice versa.

When the oligonucleotide is double-stranded and both strands of the double-stranded oligonucleotide comprise the alternating modification patterns, nucleotides of one strand can be complementary in position to nucleotides of the second strand which are similarly modified. In an alternative embodiment, there is a phase shift between the patterns of modifications of the first strand, respectively, relative to the pattern of similar modifications of the second strand. Preferably, the shift is such that the similarly modified nucleotides of the first strand and second strand are not in complementary position to each other. In some embodiments, the first strand has an alternating modification pattern wherein alternating nucleotides comprise a 2′-modification, e.g., 2′-O-Methyl modification. In some embodiments, the first strand comprises an alternating 2′-O-Methyl modification and the second strand comprises an alternating 2′-fluoro modification. In other embodiments, both strands of a double-stranded oligonucleotide comprise alternating 2′-O-methyl modifications. When both strands of a double-stranded oligonucleotide comprise alternating 2′-O-methyl modifications, such 2′-modified nucleotides can be in complementary position in the duplex region. Alternatively, such 2′-modified nucleotides may not be in complementary positions in the duplex region.

In some embodiments, the oligonucleotide comprises two chemically distinct regions, wherein each region is 1-10 (e.g., 1, 2, 3, 4, 5, 6, 7, 8, 9, or 10) nucleotides in length. In other embodiments, the oligonucleotide comprises three chemically distinct regions. The middle region is about 5-15, (e.g., 5, 6, 7, 8, 9, 10, 11, 12, 13, 14 or 15) nucleotide in length and each flanking or wing region is independently 1-10 (e.g., 1, 2, 3, 4, 5, 6, 7, 8, 9 or 10) nucleotides in length. All three regions can have different modifications or the wing regions can be similarly modified to each other. In some embodiments, the wing regions are of equal length, e.g. 1, 2, 3, 4, 5, 6, 7, 8, 9 or 10 nucleotides long.

As used herein the term “alternating motif” refers to an oligonucleotide comprising at least two different chemically distinct regions that alternate for essentially the entire sequence of the oligonucleotide. In an alternating motif length of each region is independent of the length of other regions.

As used herein, the term “uniformly fully modified motif” refers to an oligonucleotide wherein all nucleotides in the oligonucleotide have at least one modification that is the same.

As used herein, the term “hemi-mer motif” refers to an oligonucleotide having two chemically distinct regions, wherein one region is at the 5′ end of the oligonucleotide and the other region is at the 3 end of the oligonucleotide. In some embodiments, length of each chemically distinct region is independently 1 nucleotide to 1 nucleotide less than the length of the oligonucleotide.

As used herein the term “gapped motif” refers to an oligonucleotide having three chemically distinct regions. In some embodiments, the gapped motif is a symmetric gapped motif, wherein the two outer chemically distinct regions (wing regions) are identically modified. In another embodiment, the gapped motif is an asymmetric gaped motif in that the three regions are chemically distinct from each other

As used herein the term “positionally modified motif” refers to an oligonucleotide having three or more chemically distinct regions. Positionally modified oligonucleotides are distinguished from gapped motifs, hemi-mer motifs, blockmer motifs and alternating motifs because the pattern of regional substitution defined by any positional motif does not fit into the definition provided herein for one of these other motifs. The term positionally modified oligonucleotides includes many different specific substitution patterns.

In some embodiments, oligonucleotide comprises two or more chemically distinct regions and has a structure as described in International Application No. PCT/US09/038433, filed Mar. 26, 2009, content of which is incorporated herein by reference in its entirety. In some embodiments, the single-stranded oligonucleotide has a ZXY structure, such as is described in International Application No. PCT/US2004/07070 filed on Mar. 8, 2004, content of which is incorporated herein by reference in its entirety.

In some embodiments of the various aspect described herein, the oligonucleotides can include one or more oligonucleotide or nucleic acid modifications. Unmodified oligonucleotides can be less than optimal in some applications, e.g., unmodified oligonucleotides can be prone to degradation by e.g., cellular nucleases. However, chemical modifications to one or more of the subunits of oligonucleotide can confer improved properties, e.g., can render oligonucleotides more stable to nucleases. Typical oligonucleotide modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone, e.g. peptide nucleic acid (PNA); (vi) modification of the 3′ end or 5′ end of the oligonucleotide, e.g., removal, modification or replacement of a terminal phosphate group or conjugation of a moiety, e.g., conjugation of a ligand, to either the 3′ or 5′ end of oligonucleotide; and (vii) modification of the sugar, e.g., six membered rings.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule. As described below, modifications, e.g., those described herein, can be provided as asymmetrical modifications.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

Modifications of Phosphate Group:

The phosphate group in the intersugar linkage can be modified by replacing one of the oxygens with a different substituent. One result of this modification to RNA phosphate intersugar linkages can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the intersugar linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

Replacement of the Phosphate Group:

The phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′—O—C5′), thioethers (C3′-S—C5′), thioacetamido (C3′-N(H)—C(═O)—CH₂—S—C5′, C3′—O—P(O)—O—SS—C5′, C3′—CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Replacement of Ribophosphate Backbone:

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. In some embodiments, the oligonucleotide is a peptide nucleic acid, e.g., the ribophosphate backbone of the oligonucleotide is completely replaced by peptide nucleic acid (PNA).

Some exemplary intersugar linkage modifications include phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH2), diinethylene-sulfoxide (—CH₂—SO—CH₂), dimethylene-sulfone (—CH2-SO₂—CH₂), 2′-O-alkyl, and 2′-deoxy-2′-fluoro phosphorothioate.

Sugar Modifications:

An oligonucleotide can include modification of all or some of the sugar groups of the nucleic acid. For example, the 2′ position (H, DNA; or OH, RNA) can be modified with a number of different “oxy” or “deoxy” substituents. While not being bound by theory, enhanced stability is expected since the 2′-hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the oxygen at the 2′ position is connected by (CH₂)ₙ, wherein n=1-4, to the 4′ carbon of the same ribose sugar, preferably n is 1 (LNA) or 2 (ENA); O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

Examples of “deoxy” modifications include halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

The sugar group can also contain one or more carbons that possess the opposite stereochemical configuration than that of the corresponding carbon in the ribose sugar. Thus, an oligonucleotide can include nucleotides containing e.g., arabinose, as the sugar. Similarly, a modification at the 2′ position can be present in the arabinose configuration The term “arabinose configuration” refers to the placement of a substituent on the C2′ of ribose in the same configuration as the 2′-OH is in the arabinose.

A nucleotide can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Oligonucleotides can also include abasic sugars, i.e., monomers which lack a nucleobase at C-1′ or has other chemical groups in place of a nucleobase at C1′. See for example U.S. Pat. No. 5,998,203, contents of which are herein incorporated in their entirety. These abasic sugars can also be further containing modifications at one or more of the constituent sugar atoms. Oligonucleotides can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in the a configuration.

Oligonucleotide modifications can also include acyclic nucleotides, wherein a CC bonds between ribose carbons (e.g., C1′-C2′, C2′-C3′, C3′-C4′, C4′-04′, C1′-04′) is absent and/or at least one of ribose carbons or oxygen (e.g., Cr, C2′, C3′, C4′ or 04′) are independently or in combination absent from the nucleotide. In some embodiments, acyclic nucleotide is

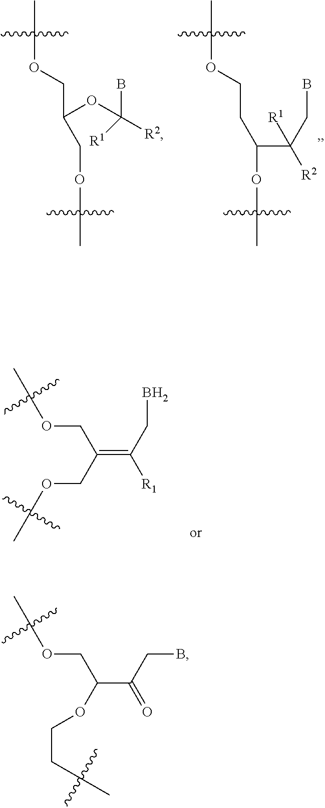

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

Preferred sugar modifications are 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), and 2′-O-dimethylaminoethyloxyethyl (2′-O-DMAEOE).

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration. The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(R′), phosphorous containing linkage, optionally substituted aryl, optionally substituted heteroaryl, optionally substituted heterocyclic or optionally substituted cycloalkyl, where R′ is hydrogen, halogen, alkyl, alkenyl, alkynyl, alkoxy, aryl, heteroaryl, cyclyl, or heterocyclyl, each of which can be optionally substituted. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the oligonucleotide.

Nucleobase Modifications:

Adenine, cytosine, guanine, thymine and uracil are the most common bases (or nucleobases) found in nucleic acids. These bases can be modified or replaced to provide oligonucleotides having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the above modifications. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligonucleotide can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, 0⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any modified or nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

Terminal Modifications:

In vivo applications of oligonucleotides can be limited due to presence of nucleases in the serum and/or blood. Thus, in certain instances it is preferable to modify the 3′, 5′ or both ends of an oligonucleotide to make the oligonucleotide resistant against exonucleases. In some embodiments, the oligonucleotide comprises a cap structure at 3′ (3′-cap), 5′ (5′-cap) or both ends. In some embodiments, oligonucleotide comprises a 3′-cap. In another embodiment, oligonucleotide comprises a 5′-cap. In yet another embodiment, oligonucleotide comprises both a 3′ cap and a 5′ cap. It is to be understood that when an oligonucleotide comprises both a 3′ cap and a 5′ cap, such caps can be same or they can be different.

As used herein, “cap structure” refers to chemical modifications, which have been incorporated at either terminus of oligonucleotide. See for example U.S. Pat. No. 5,998,203 and International Patent Publication WO03/70918, contents of which are herein incorporated in their entireties.

Exemplary 5′-caps include, but are not limited to, ligands, 5′-5′-inverted nucleotide, 5′-5′-inverted abasic nucleotide residue, 2′-5′ linkage, 5′-amino, 5′-amino-alkyl phosphate, 5′-hexylphosphate, 5′-aminohexyl phosphate, bridging and/or non-bridging 5′-phosphoramidate, bridging and/or non-bridging 5′-phosphorothioate and/or 5′-phosphorodithioate, bridging or non bridging 5′-methylphosphonate, non-phosphodiester intersugar linkage between the end two nucleotides, 4′,5′-methylene nucleotide, I-(beta-D-erythrofuranosyl) nucleotide, 4′-thio nucleotide, carbocyclic nucleotide, 1,5-anhydrohexitol nucleotide, L-nucleotides, alpha-nucleotides, modified nucleobase nucleotide, phosphorodithioate linkage, threo-pentofuranosyl nucleotide, acyclic nucleotide, acyclic 3,4-dihydroxybutyl nucleotide, acyclic 3,5-dihydroxypentyl nucleotide, 5′-mercapto nucleotide and 5′-1,4-butanediol phosphate.

Exemplary 3′-caps include, but are not limited to, ligands, 3′-3′-inverted nucleotide, 3′-3′-inverted abasic nucleotide residue, 3′-2′-inverted nucleotide moiety, 3′-2′-inverted abasic moiety, 2′-5′-linkage, 3′-amino, 3′-amino-alkyl phosphate, 3′-hexylphosphate, 3′-aminohexyl phosphate, bridging and/or non-bridging 3′-phosphoramidate, bridging and/or non-bridging 3′-phosphorothioate and/or 3′-phosphorodithioate, bridging or non bridging 3′-methylphosphonate, non-phosphodiester intersugar linkage between the end two nucleotides, I-(beta-D-erythrofuranosyl) nucleotide, 4′-thio nucleotide, carbocyclic nucleotide, 1,5-anhydrohexitol nucleotide, L-nucleotides, alpha-nucleotides, modified nucleobase nucleotide, phosphorodithioate linkage, threo-pentofuranosyl nucleotide, acyclic nucleotide, acyclic 3,4-dihydroxybutyl nucleotide, acyclic 3,5-dihydroxypentyl nucleotide, and 3′-1,4-butanediol phosphate. For more details see Beaucage and Iyer, 1993, Tetrahedron 49, 1925, incorporated by reference herein. Other 3′ and/or 5′ caps amenable to the invention are described in U.S. Provisional Application No. 61/223,665, filed Jul. 7, 2009, contents of which are herein incorporated in their entirety.

The 3′ and/or 5′ ends of an oligonucleotide can also be conjugated to other functional molecular entities such as labeling moieties, e.g., fluorophore (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

Terminal modifications useful for modulating activity include modification of the 5′ end with phosphate or phosphate analogs. For example, in some embodiments the 5′ end of the oligonucleotide can be phosphorylated or includes a phosphoryl analog at the 5′ terminus. The 5′-phosphate modifications can include those which are compatible with RISC mediated gene silencing. Modifications at the 5′-terminal end can also be useful in stimulating or inhibiting the immune system of a subject. In some embodiments, the 5′-end of the oligonucleotide comprises

the modification

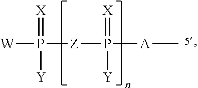

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments n is 1 or 2. It is understood that A is replacing the oxygen linked to 5′ carbon of sugar.

In some embodiments, one or both hydrogen on C5′ of the 5′-terminal nucleotides can be replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)2(X)P—O[CH₂)ₐ—P(X)(OH)—O]_{b}-5′, ((HO)₂(X)P—[—(CH₂)ₐ—O—P(X)(OH)—O]_{b}-5; dialkyl terminal phosphates and phosphate mimics: HO[CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[CH₂)ₐ—P(X)(OH)—O]_{b}-5′, H₂N[—P(CH₂)ₐ—P(X)(OH)—O]_{b}-5′, H[(CH₂)ₐ—P(X)(OH)—O]_{b}-5′, Me₂N[—(CH₂)ₐ—P(X)(OH)—O]_{b}-5′, wherein a and b are each independently 1-10. Other embodiments include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

A wide variety of entities, e.g., ligands, can be coupled to the oligonucleotides described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA) (SEQ ID NO: 28); AALAEALAEALAEALAEALAEALAAAAGGC (EALA) (SEQ ID NO: 29); ALEALAEALEALAEA (SEQ ID NO: 30); GLFEAIEGFIENGWEGMIWDYG (INF-7) (SEQ ID NO: 31); GLFGAIAGFIENGWEGMIDGWYG (Inf HA-2) (SEQ ID NO: 32); GLFEAIEGFIENGWEGMIDGWYGCGLFEAIEGFIENGWEGMID GWYGC (diINF-7) (SEQ ID NO: 33); GLFEAIEGFIENGWEGMIDGGCGLFEAIEGFIENGWEGMIDGGC (diINF-3) (SEQ ID NO: 34); GLFGALAEALAEALAEHLAEALAEALEALAAGGSC (GLF) (SEQ ID NO: 35); GLFEAIEGFIENGWEGLAEALAEALEALAAGGSC (GALA-INF3) (SEQ ID NO: 36); GLF EAI EGFI ENGW EGnI DG K GLF EAI EGFI ENGW EGnI DG (INF-5, n is norleucine) (SEQ ID NO: 37); LFEALLELLESLWELLLEA (JTS-1) (SEQ ID NO: 38); GLFKALLKLLKSLWKLLLKA (ppTG1) (SEQ ID NO: 39); GLFRALLRLLRSLWRLLLRA (ppTG20) (SEQ ID NO: 40); WEAKLAKALAKALAKHLAKALAKALKACEA (KALA) (SEQ ID NO: 41); GLFFEAIAEFIEGGWEGLIEGC (HA) (SEQ ID NO: 42); GIGAVLKVLTTGLPALISWIKRKRQQ (Melittin) (SEQ ID NO: 43); H₅WYG (SEQ ID NO: 44); and CHK₆HC (SEQ ID NO: 45).

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine.

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 2007/0036865; and 2004/0198687, content of all of is incorporated herein by reference in its entirety.

Exemplary cell permeation peptides include, but are not limited to, RQIKIWFQNRRMKWKK (penetratin) (SEQ ID NO: 46); GRKKRRQRRRPPQC (Tat fragment 48-60) (SEQ ID NO: 47); GALFLGWLGAAGSTMGAWSQPKKKRKV (signal sequence based peptide) (SEQ ID NO: 48); LLIILRRRIRKQAHAHSK (PVEC) (SEQ ID NO: 49); GWTLNSAGYLLKINLKALAALAKKIL (transportan) (SEQ ID NO: 50); KLALKLALKALKAALKLA (amphiphilic model peptide) (SEQ ID NO: 51); RRRRRRRRR (Arg9) (SEQ ID NO: 52); KFFKFFKFFK (Bacterial cell wall permeating peptide) (SEQ ID NO: 53); LLGDFFRKSKEKIGKEFKRIVQRIKDFLRNLVPRTES (LL-37) (SEQ ID NO: 54); SWLSKTAKKLENSAKKRISEGIAIAIQGGPR (cecropin P1) (SEQ ID NO: 55); ACYCRIPACIAGERRYGTCIYQGRLWAFCC (α-defensin) (SEQ ID NO: 56); DHYNCVSSGGQCLYSACPIFTKIQGTCYRGKAKCCK (β-defensin) (SEQ ID NO: 57); RRRPRPPYLPRPRPPPFFPPRLPPRIPPGFPPRFPPRFPGKR-NH2 (PR-39) (SEQ ID NO: 58); ILPWKWPWWPWRR-NH2 (indolicidin) (SEQ ID NO: 59); AAVALLPAVLLALLAP (RFGF) (SEQ ID NO: 60); AALLPVLLAAP (RFGF analogue) (SEQ ID NO: 61); and RKCRIVVIRVCR (bactenecin) (SEQ ID NO: 62).

Exemplary cationic groups include, but are not limited to, protonated amino groups, derived from e.g., 0-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactose (GalNAc), multivalent GalNAc, e.g. GalNAc2 and GalNAc3; D-mannose, multivalent mannose, multivalent lactose, N-acetyl-galactosamine, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,410,104; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the oligoncucleotide. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligonucleotides that comprise a number of phosphorothioate intersugar linkages are also known to bind to serum protein, thus short oligonucleotides, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

Ligands can be coupled to the oligonucleotides at various places, for example, 3′-end, 5′-end, and/or at an internal position. When two or more ligands are present, the ligand can be on opposite ends of an oligonucleotide. In preferred embodiments, the ligand is attached to the oligonucleotides via an intervening tether/linker. The ligand or tethered ligand can be present on a monomer when said monomer is incorporated into the growing strand. In some embodiments, the ligand can be incorporated via coupling to a “precursor” monomer after said “precursor” monomer has been incorporated into the growing strand. For example, a monomer having, e.g., an amino-terminated tether (i.e., having no associated ligand), e.g., monomer-linker-NH₂ can be incorporated into a growing oligonucleotide strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

For double-stranded oligonucleotides, ligands can be attached to one or both strands.

In some embodiments, ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligomeric compounds. Generally, an oligomeric compound is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative U.S. patents that teach the preparation of oligonucleotide conjugates include, but are not limited to, U.S. Pat. Nos. 4,828,979; 4,948,882; 5,218, 105; 5,525,465; 5,541,313; 5,545,730; 5,552,538; 5,578, 717, 5,580,731; 5,580,731; 5,591,584; 5,109,124; 5,118,802; 5,138,045; 5,414,077; 5,486,603; 5,512,439; 5,578, 718; 5,608,046; 4,587,044; 4,605,735; 4,667,025; 4,762, 779; 4,789,737; 4,824,941; 4,835,263; 4,876,335; 4,904, 582; 4,958,013; 5,082,830; 5,112,963; 5,214,136; 5,082, 830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510, 475; 5,512,667; 5,514,785; 5,565,552; 5,567,810; 5,574, 142; 5,585,481; 5,587,371; 5,595,726; 5,597,696; 5,599, 923; 5,599,928; 5,672,662; 5,688,941; 5,714,166; 6,153,737; 6,172,208; 6,300,319; 6,335,434; 6,335,437; 6,395, 437; 6,444,806; 6,486,308; 6,525,031; 6,528,631; 6,559, 279; content all of which is incorporated by reference in its entirety.

Ligand Carriers:

In some embodiments, the ligands, e.g. endosomolytic ligands, targeting ligands or other ligands, are linked to a monomer which is then incorporated into the growing oligonucleotide strand during chemical synthesis. Such monomers are also referred to as carrier monomers herein. The carrier monomer is a cyclic group or acyclic group; preferably, the cyclic group is selected from the group consisting of pyrrolidinyl, pyrazolinyl, pyrazolidinyl, imidazolinyl, imidazolidinyl, piperidinyl, piperazinyl, [1,3]-dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone. In some embodiments, the cyclic carrier monomer is based on pyrrolidinyl such as 4-hydroxyproline or a derivative thereof.

Exemplary ligands and ligand conjugated monomers amenable to the invention are described in U.S. patent application Ser. No. 10/916,185, filed Aug. 10, 2004; Ser. No. 10/946,873, filed Sep. 21, 2004; Ser. No. 10/985,426, filed Nov. 9, 2004; Ser. No. 10/833,934, filed Aug. 3, 2007; Ser. No. 11/115,989 filed Apr. 27, 2005, Ser. No. 11/119,533, filed Apr. 29, 2005; Ser. No. 11/197,753, filed Aug. 4, 2005; Ser. No. 11/944,227, filed Nov. 21, 2007; Ser. No. 12/328,528, filed Dec. 4, 2008; and Ser. No. 12/328,537, filed Dec. 4, 2008, contents which are herein incorporated in their entireties by reference for all purposes. Ligands and ligand conjugated monomers amenable to the invention are also described in International Application Nos. PCT/US04/001461, filed Jan. 21, 2004; PCT/US04/010586, filed Apr. 5, 2004; PCT/US04/011255, filed Apr. 9, 2005; PCT/US05/014472, filed Apr. 27, 2005; PCT/US05/015305, filed Apr. 29, 2005; PCT/US05/027722, filed Aug. 4, 2005; PCT/US08/061289, filed Apr. 23, 2008; PCT/US08/071576, filed Jul. 30, 2008; PCT/US08/085574, filed Dec. 4, 2008 and PCT/US09/40274, filed Apr. 10, 2009, contents which are herein incorporated in their entireties by reference for all purposes.

In some embodiments, the covalent linkages between the oligonucleotide and other components, e.g. a ligand or a ligand carrying monomer can be mediated by a linker. This linker can be cleavable linker or non-cleavable linker, depending on the application. As used herein, a “cleavable linker” refers to linkers that are capable of cleavage under various conditions. Conditions suitable for cleavage can include, but are not limited to, pH, UV irradiation, enzymatic activity, temperature, hydrolysis, elimination and substitution reactions, redox reactions, and thermodynamic properties of the linkage. In some embodiments, a cleavable linker can be used to release the oligonucleotide after transport to the desired target. The intended nature of the conjugation or coupling interaction, or the desired biological effect, will determine the choice of linker group.

As used herein, the term “linker” means an organic moiety that connects two parts of a compound. Linkers typically comprise a direct bond or an atom such as oxygen or sulfur, a unit such as NR¹, C(O), C(O)NH, C(O)O, NHC(O)O, OC(O)O, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, NR¹, C(O), C(O)NH, C(O)O, NHC(O)O, OC(O)O, SO₂NH, cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, the linker is a branched linker. The branchpoint of the branched linker may be at least trivalent, but can be a tetravalent, pentavalent or hexavalent atom, or a group presenting such multiple valencies. In some embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branchpoint is glycerol or derivative thereof.

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In some embodiments, cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions).

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid celavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleaveable in an acidic environment with a pH of about 6.5 or lower (e.g., about 6.5, 6.0, 5.5, 5.0, or lower), or by agents such as enzymes that can act as a general acid.

General references for oligonucleotide modification are discussed below. The anti-miR-130/301 agents and oligonucleotides used in accordance with this invention can be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3, 2′-O-Methyloligoribonucleotides: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Hell). Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

Modification of the Phosphate Group References:

The preparation of phosphinate oligonucleotides is described in U.S. Pat. No. 5,508,270. The preparation of alkyl phosphonate oligonucleotides is described in U.S. Pat. No. 4,469,863. The preparation of phosphoramidite oligonucleotides is described in U.S. Pat. No. 5,256,775 or U.S. Pat. No. 5,366,878. The preparation of phosphotriester oligonucleotides is described in U.S. Pat. No. 5,023,243. The preparation of boranophosphate oligonucleotide is described in U.S. Pat. Nos. 5,130,302 and 5,177,198. The preparation of 3′-Deoxy-3′-amino phosphoramidate oligonucleotides is described in U.S. Pat. No. 5,476,925. 3′-Deoxy-3′-methylenephosphonate oligonucleotides is described in An, H, et al. J. Org. Chem. 2001, 66, 2789-2801. Preparation of sulfur bridged nucleotides is described in Sproat et al. Nucleosides Nucleotides 1988, 7, 651 and Crosstick et al. Tetrahedron Lett. 1989, 30, 4693.

Methylenemethylimino linked oligonucleosides, also identified herein as MMI linked oligonucleosides, methylenedimethylhydrazo linked oligonucleosides, also identified herein as MDH linked oligonucleosides, and methylenecarbonylamino linked oligonucleosides, also identified herein as amide-3 linked oligonucleosides, and methyleneaminocarbonyl linked oligonucleosides, also identified herein as amide-4 linked oligonucleosides as well as mixed intersugar linkage compounds having, as for instance, alternating MMI and PO or PS linkages can be prepared as is described in U.S. Pat. Nos. 5,378,825, 5,386,023, 5,489,677 and in International Application Nos. PCT/US92/04294 and PCT/US92/04305 (published as WO 92/20822 WO and 92/20823, respectively). Formacetal and thioformacetal linked oligonucleosides can be prepared as is described in U.S. Pat. Nos. 5,264,562 and 5,264,564. Ethylene oxide linked oligonucleosides can be prepared as is described in U.S. Pat. No. 5,223,618. Siloxane replacements are described in Cormier,J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 1 1972, 1991. Carbamate replacements are described in Stirchak, E. P. Nucleic Acids Res. 1989, 17, 6129.

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They can also be prepared in accordance with U.S. Pat. No. 5,539,083.

Methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH2), diinethylene-sulfoxide (—CH₂—SO—CH₂), dimethylene-sulfone (—CH2-SO₂—CH₂), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., 5, 714, 606 to Acevedo, et al, U.S. Pat. No. 5,378,825 to Cook, et al., U.S. Pat. Nos. 5,672,697 and 5,466,786 to Buhr, et al., U.S. Pat. No. 5,777,092 to Cook, et al., U.S. Pat. No. 5,602,240 to De Mesmacker, et al., U.S. Pat. No. 5,610,289 to Cook, et al. and U.S. Pat. No. 5,858,988 to Wang, also describe nucleic acid analogs for enhanced nuclease stability and cellular uptake.

Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety.

Terminal Modification References:

Terminal modifications are described in Manoharan, M. et al. Antisense and Nucleic Acid Drug Development 12, 103-128 (2002) and references therein.

As oligonucleotides are polymers of subunits or monomers, many of the modifications described herein can occur at a position which is repeated within an oligonucleotide, e.g., a modification of a nucleobase, a sugar, a phosphate moiety, or the non-bridging oxygen of a phosphate moiety. It is not necessary for all positions in a given oligonucleotide to be uniformly modified, and in fact more than one of the aforementioned modifications can be incorporated in a single oligonucleotide or even at a single nucleoside within an oligonucleotide.

In some cases the modification will occur at all of the subject positions in the oligonucleotide but in many, and in fact in most cases it will not. By way of example, a modification can occur at a 3′ or 5′ terminus position, can occur in the internal region, can occur in 3′, 5′ or both terminal regions, e.g. at a position on a termus nucleotide or in the terminal 2, 3, 4, 5, 6, 7, 8, 9, or 10 nucleotides of an oligonucleotide. In some embodiments, the terminus nucleotide does not comprise a modification.

In some embodiments, the terminus nucleotide or the terminal 2, 3, 4, 5, 6, 7, 8, 9, or 10 nucleotides of at least one end of the oligonucleotide all comprise at least one modification. In some embodiments, the modification is same. In some embodiments, the terminus nucleotide or the last 2, 3, 4, 5, 6, 7, 8, 9, or 10 nucleotides at both ends of the oligonucleotide all comprise at least one modification. It is to be understood that type of modification and number of modified nucleotides on one end of the oligonucleotide is independent of type of modification and number of modified nucleotides on the other end of the oligonucleotide.

When the oligonucleotide is double-stranded or partially double-stranded, a modification can occur in the double strand region, the single strand region, or in both the double- and single-stranded regions. In some embodiments, a modification described herein does not occur in the region corresponding to the target cleavage site region. For example, a phosphorothioate modification at a non-bridging oxygen position can occur at one or both termini, can occur in a terminal regions, e.g., at a position on a terminal nucleotide or in the last 2, 3, 4, 5, 6, 7, 8, 9, or 10 nucleotides of a strand, or can occur in double strand and single strand regions, particularly at termini.

Some modifications can preferably be included on an oligonucleotide at a particular location, e.g., at an internal position of a strand, or on the 5′ or 3′ end of an oligonucleotide. A preferred location of a modification on an oligonucleotide can confer preferred properties on the oligonucleotide. For example, preferred locations of particular modifications can confer increased resistance to endonuclease or exonuclease activity.

In some embodiments, the oligonucleotide comprises a mix of LNA and DNA monomers, e.g., a LNA/DNA mixmer. The LNA and DNA monomers can be arranged in any pattern. In some embodiments, the LNA and DNA monomers are arranged in an alternative pattern, e.g., a LNA monomer followed by a DNA monomer. This alternating pattern can be repeated for the full length of the oligonucleotide.

In some embodiments, the oligonucleotide comprises at least 1 (e.g., 1, 2, 3, 4, or 5) LNA nucleotides at the 5′ end (i.e., the first 1, 2, 3, 4, or 5 nucleotides at the 5′ end are LNA nucleotides). In some embodiments, the oligonucleotide comprises at least 1 (e.g., 1, 2, 3, 4, or 5) LNA nucleotides at the 3′ end (i.e., the first 1, 2, 3, 4, or 5 nucleotides at the 3′ end are LNA nucleotides). In some embodiments, the oligonucleotide comprises LNA nucleotides at all positions. In some embodiments, the oligonucleotide comprises LNA nucleotides at all positions and phosphorothioate inter-sugar linkages at all positions.

In some embodiments, the oligonucleotide comprises 2′-MOE modifications at all positions and phosphorothioate inter-sugar linkages at all positions.

In some embodiments, the oligonucleotide comprises a mix of 2′-F and 2′-MOE modified nucleotides.

In some embodiments, the oligonucleotide comprises at least 1 (e.g., 1, 2, 3, 4, or 5) 2′-F modified nucleotides at the 5′ end (i.e., the first 1, 2, 3, 4, or 5 nucleotides at the 5′ end are 2′-F modified nucleotides). In some embodiments, the oligonucleotide comprises at least 1 (e.g., 1, 2, 3, 4, or 5) 2′-F modified nucleotides at the 3′ end (i.e., the first 1, 2, 3, 4, or 5 nucleotides at the 3′ end are 2′-F modified nucleotides).

In some embodiments, the oligonucleotide comprises, independently, at least 1 (e.g., 1, 2, 3, 4, or 5) 2′-F modified nucleotides at the 5′ end and at the 3′ end and 2′-MOE modified nucleotides at all other positions.

In some embodiments, the oligonucleotide comprises two 2′-F modified nucleotides at the 5′ end and at the 3′ end and 2′-MOE modified nucleotides at all other positions, e.g., a 2′-F/2′-MOE mixmer.

In some embodiments, the oligonucleotide comprises two 2′-F modified nucleotides at the 5′ end and at the 3′ end, 2′-MOE modified nucleotides at all other positions, and phosphorothioate inter-sugar linkages at all positions.

The oligonucleotides described herein can be prepared using solution-phase or solid-phase organic synthesis, or enzymatically by methods known in the art. Organic synthesis offers the advantage that the oligonucleotide strands comprising non-natural or modified nucleotides can be easily prepared. Any other means for such synthesis known in the art can additionally or alternatively be employed. It is also known to use similar techniques to prepare other oligonucleotides, such as the phosphorothioates, phosphorodithioates and alkylated derivatives. The double-stranded oligonucleotide compounds of the invention can be prepared using a two-step procedure. First, the individual strands of the double-stranded molecule are prepared separately. Then, the component strands are annealed.

Regardless of the method of synthesis, the oligonucleotide can be prepared in a solution (e.g., an aqueous and/or organic solution) that is appropriate for formulation. For example, the oligonucleotide preparation can be precipitated and redissolved in pure double-distilled water, and lyophilized. The dried oligonucleotide can then be resuspended in a solution appropriate for the intended formulation process.

Teachings regarding the synthesis of particular modified oligonucleotides can be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N−2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having beta-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups can be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N−2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

Additionally, in some embodiments, the oligonucleotides can be recombinantly produced or synthesized in vitro by a variety of techniques well known to one of ordinary skill in the art. For example, the oligonucleotide can be obtained by preparing a recombinant version thereof (i.e., by using the techniques of genetic engineering to produce a recombinant nucleic acid which can then be isolated or purified by techniques well known to one of ordinary skill in the art). This approach involves growing a culture of host cells in a suitable culture medium, and purifying the oligonucleotide from the cells or the culture in which the cells are grown. For example, the methods include a process for producing an oligonucleotide in which a host cell, containing a suitable expression vector that includes a nucleic acid encoding the oligonucleotide, is cultured under conditions that allow expression of the encoded oligonucleotide. The oligonucleotide can be recovered from the culture, from the culture medium or from a lysate prepared from the host cells, and further purified. The host cell can be a higher eukaryotic host cell such as a mammalian cell, a lower eukaryotic host cell such as a yeast cell, or the host cell can be a prokaryotic cell such as a bacterial cell. Introduction of a vector containing the nucleic acid encoding the oligonucleotide into the host cell can be effected by calcium phosphate transfection, DEAE-dextran mediated transfection, or electroporation (Davis, L. et al., Basic Methods in Molecular Biology (1986)).

Any host/vector system can be used to express one or more of the oligonucleotides. These include, but are not limited to, eukaryotic hosts such as HeLa cells and yeast, as well as prokaryotic host such as E. coli and B. subtilis. An anti-miR-130/301 agent can be expressed in mammalian cells, yeast, bacteria, or other cells where the anti-miR-130/301 agent is under the control of an appropriate promoter. Appropriate cloning and expression vectors for use with prokaryotic and eukaryotic hosts are described by Sambrook et al., Molecular Cloning: A Laboratory Manual, 4^{th} Ed, Vols 1 to 3, Cold Spring Harbor, N.Y. (2012). In the preferred embodiment, the miRNA is expressed in mammalian cells. Examples of mammalian expression systems include C127, monkey COS cells, Chinese Hamster Ovary (CHO) cells, human kidney 293 cells, human epidermal A43 1 cells, human Colo205 cells, 3T3 cells, CV-1 cells, other transformed primate cell lines, normal diploid cells, cell strains derived from in vitro culture of primary tissue, primary explants, HeLa cells, mouse L cells, BILK, HL-60, U937, HaK or Jurkat cells.

Mammalian expression vectors will comprise an origin of replication, a suitable promoter, polyadenylation site, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements. Potentially suitable yeast strains include Saccharomyces cerevsiae, Schizosaccharomyces pombe, Klayveromyces strains, Candida, or any yeast strain capable of expressing miRNA. Potentially suitable bacterial strains include Escherichia coli, Bacillus subtilis, Salmonella typhimurium, or any bacterial strain capable of expressing miRNA.

In another approach, genomic DNA encoding an anti-miR-130/301 agent is isolated, the genomic DNA is expressed in a mammalian expression system, and anti-miR-130/301 agent is purified and modified as necessary for administration to a subject.

### Delivery of Anti-miR-130/301 Agents

In general, any method of delivering a nucleic acid molecule can be adapted for use with the anti-miR130/301 agents described herein (see e.g., Akhtar S, and Julian R L., 1992, Trends Cell. Biol. 2 (5):139-144 and WO94/02595, which are incorporated herein by reference in their entireties). However, there are three factors that are important to consider in order to successfully delivering a nucleic acid agent in vivo: (a) biological stability of the delivered molecule, (2) preventing non-specific effects, and (3) accumulation of the delivered molecule in the target tissue. The non-specific effects of a nucleic acid agent can be minimized by local administration, for example by direct injection or implantation into a tissue (as a non-limiting example, a tumor) or topically administering the preparation. Local administration to a treatment site maximizes local concentration of the agent, limits the exposure of the agent to systemic tissues that may otherwise be harmed by the agent or that can degrade the agent, and permits a lower total dose of the agent to be administered. Several studies have shown successful knockdown of gene products when an iRNA agent is administered locally. For example, intraocular delivery of a VEGF dsRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of a dsRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering a nucleic acid agent systemically for the treatment of a disease, the nucleic acid agent can be modified or alternatively delivered using a drug delivery system; both methods act to prevent the rapid degradation of the nucleic acid agent by endo- and exo-nucleases in vivo. In an alternative embodiment, the nucleic acid agent can be delivered using drug delivery systems such as a nanoparticle, a dendrimer, a polymer, liposomes, or a cationic delivery system. Positively charged cationic delivery systems facilitate binding of a nucleic acid molecule (negatively charged) and also enhance interactions at the negatively charged cell membrane to permit efficient uptake of the nucleic acid agent by the cell. Cationic lipids, dendrimers, or polymers can either be bound to a nucleic acid agent, or induced to form a vesicle or micelle (see e.g., Kim S H., et al (2008) Journal of Controlled Release 129 (2):107-116) that encases a nucleic acid agent. The formation of vesicles or micelles further prevents degradation of the nucleic acid agent when administered systemically. Methods for making and administering cationic-nucleic acid complexes are well within the abilities of one skilled in the art (see e.g., Sorensen, D R., et al (2003) J. Mol. Biol. 327:761-766; Verma, U N., et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of nucleic acids and cyclodextrins can be found in U.S. Pat. No. 7,427,605, which is herein incorporated by reference in its entirety.

In another aspect, an anti-miR-130/301 agent can be expressed from transcription units inserted into DNA or RNA vectors (see, e.g., Couture, A, et al., TIG., 1996, 12:5-10; Skillern, A., et al., International PCT Publication No. WO 00/22113, Conrad, PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA, 1995, 92:1292).

The individual strand or strands of a dsRNA can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

RNA expression vectors are generally DNA plasmids or viral vectors. Expression vectors compatible with eukaryotic cells, preferably those compatible with vertebrate cells, can be used to produce recombinant constructs for the expression of an iRNA as described herein. Eukaryotic cell expression vectors are well known in the art and are available from a number of commercial sources. Typically, such vectors are provided containing convenient restriction sites for insertion of the desired nucleic acid segment. Delivery of iRNA expressing vectors can be systemic, such as by intravenous or intramuscular administration, by administration to target cells ex-planted from the patient followed by reintroduction into the patient, or by any other means that allows for introduction into a desired target cell. Vectors are described below in more detail.

As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al., Molecular Cloning: A Laboratory Manual, 4^{th} Ed, Vols 1 to 3, Cold Spring Harbor, N.Y. (2012). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′-non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, 4^{th} Ed, Vols 1 to 3, Cold Spring Harbor, N.Y. (2012).

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g EPV and EBV vectors. Constructs for the recombinant expression of an iRNA will generally require regulatory elements, e.g., promoters, enhancers, etc., to ensure the expression of the anti-miR-130/301 agent in target cells. Other aspects to consider for vectors and constructs are further described below.

Vectors useful for the delivery of anti-miR-130/301 agent will include regulatory elements (promoter, enhancer, etc.) sufficient for expression of the anti-miR-130/301 agent in the desired target cell or tissue. The regulatory elements can be chosen to provide either constitutive or regulated/inducible expression.

Expression of the anti-miR-130/301 agent can be precisely regulated, for example, by using an inducible regulatory sequence that is sensitive to certain physiological regulators, e.g., circulating glucose levels, or hormones (Docherty et al., 1994, FASEB J. 8:20-24). Such inducible expression systems, suitable for the control of dsRNA expression in cells or in mammals include, for example, regulation by ecdysone, by estrogen, progesterone, tetracycline, chemical inducers of dimerization, and isopropyl-beta-D1-thiogalactopyranoside (IPTG). A person skilled in the art would be able to choose the appropriate regulatory/promoter sequence based on the intended use of the iRNA transgene.

In a specific embodiment, viral vectors that contain nucleic acid sequences encoding the anti-miR-130/301 agent can be used. For example, a retroviral vector can be used. These retroviral vectors contain the components necessary for the correct packaging of the viral genome and integration into the host cell DNA. More detail about retroviral vectors can be found, for example, in Boesen et al., Biotherapy 6:291-302 (1994), which describes the use of a retroviral vector to deliver the mdrl gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). Lentiviral vectors contemplated for use include, for example, the HIV based vectors described in U.S. Pat. Nos. 6,143,520; 5,665,557; and 5,981,276, which are herein incorporated by reference.

Adenoviruses are also contemplated for use in delivery of anti-miR-130/301 agents. Adenoviruses are especially attractive vehicles, e.g., for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995).

Use of Adeno-associated virus (AAV) vectors is also contemplated (Walsh et al., Proc. Soc. Exp. Biol. Med. 204:289-300 (1993); U.S. Pat. No. 5,436,146). In one embodiment, the CD33 protein can be expressed from a recombinant AAV vector having, for example, either the U6 or H1 RNA promoters, or the cytomegalovirus (CMV) promoter. Suitable AAV vectors for expressing the CD33 protein, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

Another preferred viral vector is a pox virus such as a vaccinia virus, for example an attenuated vaccinia such as Modified Virus Ankara (MVA) or NYVAC, an avipox such as fowl pox or canary pox.

### Formulations

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. Thus, in some embodiments, the anti-miR-130/301 agent or an expression encoding the same can be formulated as a liposome. As used herein, a liposome is a structure having lipid-containing membranes enclosing an aqueous interior. Generally, liposomes are a vesicle composed of amphiphilic lipids arranged in a spherical bilayer or bilayers. Liposomes can have one or more lipid membranes. In some embodiments, liposomes have an average diameter of less than about 100 nm. More preferred embodiments provide liposomes having an average diameter from about 30-70 nm and most preferably about 40-60 nm. Oligolamellar large vesicles and multilamellar vesicles have multiple, usually concentric, membrane layers and are typically larger than 100 nm. Liposomes with several nonconcentric membranes, i.e., several smaller vesicles contained within a larger vesicle, are termed multivesicular vesicles.

Liposomes can further comprise one or more additional lipids and/or other components such as sterols, e.g., cholesterol. Additional lipids can be included in the liposome compositions for a variety of purposes, such as to prevent lipid oxidation, to stabilize the bilayer, to reduce aggregation during formation or to attach ligands onto the liposome surface. Any of a number of additional lipids and/or other components can be present, including amphipathic, neutral, cationic, anionic lipids, and programmable fusion lipids. Such lipids and/or components can be used alone or in combination. One or more components of the liposome can comprise a ligand, e.g., a targeting ligand.

In order to traverse intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent can act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al. S. T. P. Pharma. Sci., 1994, 4(6):466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describes PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes can include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

Liposome compositions can be prepared by a variety of methods that are known in the art. See e.g., U.S. Pat. Nos. 4,235,871; 4,737,323; 4,897,355 and 5,171,678; published International Applications WO 96/14057 and WO 96/37194; Felgner, P. L. et al., Proc. Natl. Acad. Sci., USA (1987) 8:7413-7417, Bangham, et al. M Mol. Biol. (1965) 23:238, Olson, et al. Biochim. Biophys. Acta (1979) 557:9, Szoka, et al. Proc. Natl. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757.

Transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

In some embodiments, the anti-miR-130/301 agent or an expression vector encoding same can be fully encapsulated in the lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964. For SNALP formulation, the particle size is at least 30 nm, at least 40 nm, at least 50 nm, at least 60 nm, at least 70 nm, at least 80 nm, at least 90 nm, at least 100 nm, at least 110 nm, and at least 120 nm. The suitable range is typically about at least 50 nm to about at least 110 nm, about at least 60 nm to about at least 100 nm, or about at least 80 nm to about at least 90 nm.

In one embodiment, the lipid to drug ratio (mass/mass ratio) (e.g., lipid to nucleic acid) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid can be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech Gi), or a mixture thereof. The cationic lipid can comprise from about 20 mol % to about 50 mol % or about 40 mol % of the total lipid present in the particle.

The non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C]₈). The conjugated lipid that prevents aggregation of particles can be from 0.01 mol % to about 20 mol % or about 2 mol % of the total lipid present in the particle.

In some embodiments, the nati-miR-130/301 agent, or an expression vector encoding same can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y. Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials can also be included in emulsion formulations and can contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment the anti-miR-130/301 agent or an expression vector encoding same can be formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems,

Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (P0310), hexaglycerol pentaoleate (P0500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of anti-miR-130/301 agents or expression vectors encoding same from the gastrointestinal tract, as well as improve the local cellular uptake of anti-miR-130/301 agents or expression vectors.

In some embodiments, the anti-miR-130/301 agent or expression vector encoding same can be prepared and formulated as micelles. As used herein, “micelles” are a particular type of molecular assembly in which amphipathic molecules are arranged in a spherical structure such that all hydrophobic portions on the molecules are directed inward, leaving the hydrophilic portions in contact with the surrounding aqueous phase. The converse arrangement exists if the environment is hydrophobic.

In some embodiments, the formulations comprises micelles formed from an anti-miR-130/301 agent or expression vector encoding same and at least one amphiphilic carrier, in which the micelles have an average diameter of less than about 100 nm, preferably. More preferred embodiments provide micelles having an average diameter less than about 50 nm, and even more preferred embodiments provide micelles having an average diameter less than about 30 nm, or even less than about 20 nm.

Micelle formulations can be prepared by mixing an aqueous solution of the anti-miR-130/301 agent or expression vector encoding same, an alkali metal C₈ to C₂₂ alkyl sulphate, and an amphiphilic carrier. The amphiphilic carrier can be added at the same time or after addition of the alkali metal alkyl sulphate. Micelles will form with substantially any kind of mixing of the ingredients but vigorous mixing in order to provide smaller size micelles.

In some embodiments, the the anti-miR-130/301 agent or expression vector encoding same can be prepared and formulated as lipid particles, e.g., formulated lipid particles (FLiPs) comprising (a) an anti-miR-130/301 agent or expression vector encoding same, where said anti-miR-130/301 agent or expression vector encoding same has been conjugated to a lipophile and (b) at least one lipid component, for example an emulsion, liposome, isolated lipoprotein, reconstituted lipoprotein or phospholipid, to which the conjugated anti-miR-130/301 agent or expression vector encoding same has been aggregated, admixed or associated. The stoichiometry of anti-miR-130/301 agent or expression vector encoding same to the lipid component can be 1:1. Alternatively the stoichiometry can be 1:many, many:1 or many:many, where many is two or more.

The FLiP can comprise triacylglycerols, phospholipids, glycerol and one or several lipid-binding proteins aggregated, admixed or associated via a lipophilic linker molecule with an oligonucleotide. Surprisingly, it has been found that due to said one or several lipid-binding proteins in combination with the above mentioned lipids, the FLiPs show affinity to liver, gut, kidney, steroidogenic organs, heart, lung and/or muscle tissue. These FLiPs can therefore serve as carrier for oligonucleotides to these tissues. For example, lipid-conjugated oligonucleotides, e.g., cholesterol-conjugated oligonucleotides, bind to HDL and LDL lipoprotein particles which mediate cellular uptake upon binding to their respective receptors thus directing oligonucleotide delivery into liver, gut, kidney and steroidogenic organs, see Wolfrum et al. Nature Biotech. (2007), 25:1145-1157.

The FLiP can be a lipid particle comprising 15-25% triacylglycerol, about 0.5-2% phospholipids and 1-3% glycerol, and one or several lipid-binding proteins. FLiPs can be a lipid particle having about 15-25% triacylglycerol, about 1-2% phospholipids, about 2-3% glycerol, and one or several lipid-binding proteins. In some embodiments, the lipid particle comprises about 20% triacylglycerol, about 1.2% phospholipids and about 2.25% glycerol, and one or several lipid-binding proteins.

Another suitable lipid component for FLiPs is lipoproteins, for example isolated lipoproteins or more preferably reconstituted lipoprotieins. Exemplary lipoproteins include chylomicrons, VLDL (Very Low Density Lipoproteins), IDL (Intermediate Density Lipoproteins), LDL (Low Density Lipoproteins) and HDL (High Density Lipoproteins). Methods of producing reconstituted lipoproteins are known in the art, for example see A. Jones, Experimental Lung Res. 6, 255-270 (1984), U.S. Pat. Nos. 4,643,988 and 5,128,318, PCT publication WO87/02062, Canadian Pat. No. 2,138,925. Other methods of producing reconstituted lipoproteins, especially for apolipoproteins A-I, A-II, A-IV, apoC and apoE have been described in A. Jonas, Methods in Enzymology 128, 553-582 (1986) and G. Franceschini et al. J. Biol. Chem., 260(30), 16321-25 (1985).

One preferred lipid component for FLiP is Intralipid. Intralipid® is a brand name for the first safe fat emulsion for human use. Intralipid® 20% (a 20% intravenous fat emulsion) is made up of 20% soybean oil, 1.2% egg yolk phospholipids, 2.25% glycerin, and water for injection. It is further within the present invention that other suitable oils, such as saflower oil, can serve to produce the lipid component of the FLiP.

FLiP can range in size from about 20-50 nm or about 30-50 nm, e.g., about 35 nm or about 40 nm. In some embodiments, the FLiP has a particle size of at least about 100 nm. FLiPs can alternatively be between about 100-150 nm, e.g., about 110 nm, about 120 nm, about 130 nm, or about 140 nm, whether characterized as liposome- or emulsion-based. Multiple FLiPs can also be aggregated and delivered together; therefore the size can be larger than 100 nm.

The process for making the lipid particles comprises the steps of: (a) mixing a lipid component with one or several lipophile (e.g. cholesterol) anti-miR-130/301 agents or expression vectors encoding same that can be chemically modified; and (b) fractionating this mixture. In some embodiments, the process comprises the additional step of selecting the fraction with particle size of 30-50 nm, preferably of about 40 nm in size.

Some exemplary lipid particle formulations amenable to the invention are described in U.S. patent application Ser. No. 12/412,206, filed Mar. 26, 2009, contents of which are herein incorporated by reference in their entirety.

Yeast cell wall particles: In some embodiments, the anti-miR-130/301 agent or expression vector encoding same can be formulated in yeast cell wall particles (“YCWP”). A yeast cell wall particle comprises an extracted yeast cell wall exterior and a core, the core comprising a payload (e.g., anti-miR-130/301 agent or expression vector encoding same). Exterior of the particle comprises yeast glucans (e.g. beta glucans, beta-1,3-glucans, beta-1,6-glucans), yeast mannans, or combinations thereof. Yeast cell wall particles are typically spherical particles about 1-4 μm in diameter.

Preparation of yeast cell wall particles is known in the art, and is described, for example in U.S. Pat. Nos. 4,992,540; 5,082,936; 5,028,703; 5,032,401; 5,322,841; 5,401,727; 5,504,079; 5,607,677; 5,741,495; 5,830,463; 5,968,811; 6,444,448; and 6,476,003, U.S. Pat. App. Pub. Nos. 2003/0216346 and 2004/0014715, and Int. App. Pub. No. WO 2002/12348, contents of which are herein incorporated by reference in their entirety. Applications of yeast cell like particles for drug delivery are described, for example in U.S. Pat. Nos. 5,032,401; 5,607,677; 5,741,495; and 5,830,463, and U.S. Pat. Pub Nos. 2005/0281781 and 2008/0044438, contents of which are herein incorporated by reference in their entirety. U.S. Pat. App. Pub. No. 2009/0226528, contents of which are herein incorporated by reference, describes formulation of nucleic acids with yeast cell wall particles for delivery of oligonucleotide to cells.

Delivery of 2′OMe- and PS-modified 21-nt siRNAs to the lung has been demonstrated with a methoxypolyethylene glycol (mPEG)-functionalized lipopolyamine (Staramine) delivery system following i.v. administration. (Polach, K J, Matar, M, Rice, J, Slobodkin, G, Sparks, J, Congo, R et al. (2011). Delivery of siRNA to the Mouse Lung via a Functionalized Lipopolyamine. Mol Ther, 2012, vol. 20 (1), pp 91-100, content of which is incorporated herein by reference in its entirety) Biodistribution studies showed the lung retained the greatest amount of siRNA at 24 and 48 hours when compared to liver, kidney and spleen on a per gram of tissue basis. Staramine functionalized with a monodisperse (mPEG) (515 Da), as opposed to the polydisperse form, further improved lung targeting. In an attempt to identify the lung cell type targeted by these i.v.-dosed nanocomplexes. Accordingly, in some embodiments, the anti-miR-130/301 agent or expression vector encoding same can be formulated in a Staramine nanoparticle delivery system. The Staramine delivery system (nanocomplex) comprises a lipopolyamine (Staramine) comprising a head group containing a free amine, an endosomalytic core and lipid tails. The amine group allows for covalent attachment of molecules of interest, such as methoxy polyethylene glycol (Staramine-mPEG), that can modify the in vivo behavior of the nanocomplex. Structures of Staramine and mPEG modified Staramine are shown in Polach et al. Useful lipopolymaines similar to Staramine are also described, for example, in Int. Patent Application Publication No. WO2010108108, content of which is incorporated herein by reference.

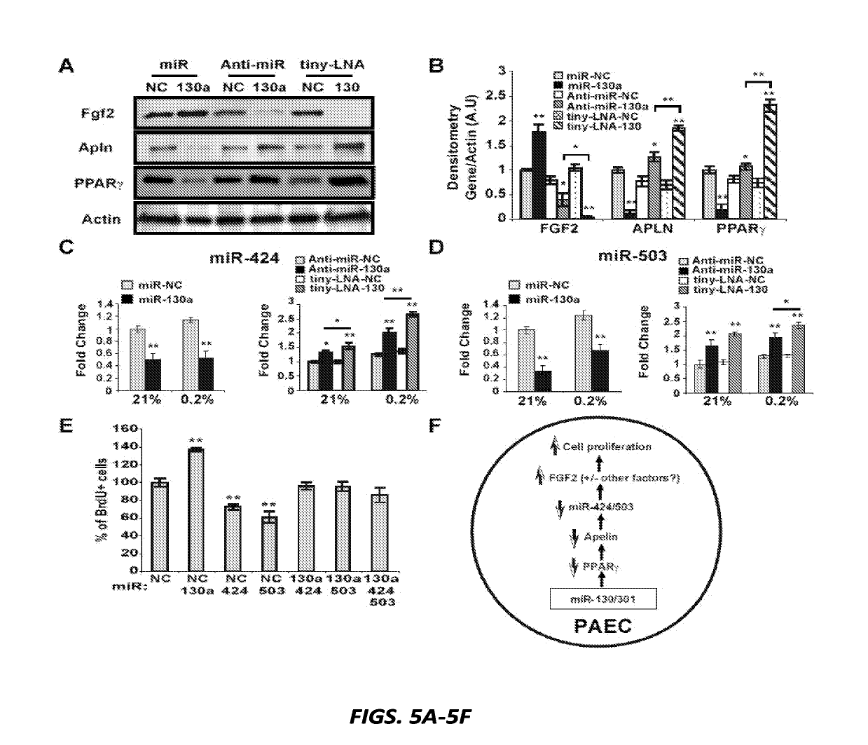

In some embodiments, anti-miR-130/301 agents can be formulated as golden lipid nanoparticles for therapeutic delivery, e.g., as disclosed in 2012/0128777, 2012/0244075 and 2013/0011339, and in Shi et al., Solid lipid nanoparticles loaded with anti-microRNA oligonucleotides (AMOs) for suppression of microRNA-21 functions in human lung cancer cells, Pharm Res, 2012; 29(1); 97-109, which are incorporated herein in its entirety by reference. In some embodiments, chemical modifications that improve the stability, biodistribution and delivery of anti-miR-130/301 agents is encompassed, for example as disclosed in FIG. 5 in Broderick et al., Gene Therapy, 2011, 18; 1104-1110.

In one embodiment, a vector encoding an anti-miR-130/301 agent is delivered into a specific target cell. Nucleic acid sequences necessary for expression in prokaryotes usually include a promoter, an operator (optional), often along with other sequences. Eukaryotic cells are known to utilize promoters, enhancers, and termination and polyadenylation signals.

One can also use localization sequences to deliver and release an anti-miR-130/301 agent intracellularly to a cell compartment of interest. Typically, the delivery system first binds to a specific receptor on the cell. Thereafter, the targeted cell internalizes the delivery system, which is bound to the cell. For example, membrane proteins on the cell surface, including receptors and antigens can be internalized by receptor mediated endocytosis after interaction with the ligand to the receptor or antibodies. (Dautry-Varsat, A., et al., Sci. Am. 250:52-58 (1984)). One can also include sequences or moieties that disrupt endosomes and lysosomes. See, e.g., Cristiano, R. J., et al., Proc. Natl. Acad. Sci. USA 90:11548-11552 (1993); Wagner, E., et al., Proc. Natl. Acad. Sci. USA 89:6099-6103 (1992); Cotten, M., et al., Proc. Natl. Acad. Sci. USA 89:6094-6098 (1992).

In some embodiments, an anti-miR-130/301 agent can be complexed with desired targeting moieties by mixing an anti-miR-130/301 with the targeting moiety in the presence of complexing agents. Examples of such complexing agents include, but are not limited to, poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. In some embodiments, the complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DE AE-albumin and DEAE-dextran, polymethylacrylate, polyhexylacrylate, poly(D,L-lactic acid), poly(DL-lactic-co-glycolic acid (PLGA), alginate, and polyethyleneglycol (PEG), and polyethylenimine.

In alternative embodiments, an anti-miR-130/301 complexing agent is protamine or an RNA-binding domain, such as an siRNA-binding fragment or nucleic acid binding fragment of protamine. Protamine is a polycationic peptide with molecular weight about 4000-4500 Da. Protamine is a small basic nucleic acid binding protein, which serves to condense the animal's genomic DNA for packaging into the restrictive volume of a sperm head (Warrant, R. W., et al., Nature 271:130-135 (1978); Krawetz, S. A., et al., Genomics 5:639-645 (1989)). The positive charges of the protamine can strongly interact with negative charges of the phosphate backbone of nucleic acid, such as RNA, resulting in a neutral and stable interference RNA-protamine complex. In one embodiment, the protamine fragment is encoded by a nucleic acid sequence disclosed in International Patent Application: PCT/US05/029111, which is incorporated herein in its entirety by reference. The methods, reagents and references that describe a preparation of a nucleic acid-protamine complex in detail are disclosed in the U.S. Patent Application Publication Nos. US2002/0132990 and US2004/0023902, and are herein incorporated by reference in their entirety.

In some embodiments of the various aspects disclosed herein, the anti-miR-130/301 agent is targeted to specific cells, for example cells expressing one or more members of miR-103/301 family. However, the fact that miR-130/301 is present in the plasma of subjects, it is not essential to have the anti-miR-103/301 agent targeted to any particular cell, as an anti-miR-103/301 present in the plasma will have effect at inhibiting the effect of miR-103/301 circulating in the blood. In some embodiments, where the anti-miR-130/301 agent is an anti-miR, the anti-miR can be fused to a cell targeting moiety or protein, as disclosed in the International Patent Application PCT/US05/029111 which is incorporated herein in its entirety by reference. In such embodiments, the target moiety specifically brings the delivery system to the target cell. The particular target moiety for delivering the anti-miR-130/301 agent can be determined empirically based upon the present disclosure and depending upon the target cell.

In some embodiments, the anti-miR-130/301 agent can be delivered to a limited number of cells thereby limiting, for example, potential side effects of therapies using the anti-miR-130/301 agent. The particular cell surface targets that are chosen for the targeting moiety will depend upon the target cell. Cells can be specifically targeted, for example, by use of antibodies against unique proteins, lipids or carbohydrates that are present on the cell surface. A skilled artisan can readily determine such molecules based on the general knowledge in the art.

The strategy for choosing the targeting moiety is very adaptable. For example, any cell-specific antigen, including proteins, carbohydrates and lipids can be used to create an antibody that can be used to target the anti-miR-130/301 agent to a specific cell type according to the methods described herein.

In some embodiments, a binding domain is used to complex the targeting moiety to an anti-miR-130/301 agent. In some embodiments, the binding domain is selected from the nucleic acid binding domains present in proteins selected from the group consisting of GCN4, Fos, Jun, TFIIS, FMRI, yeast protein HX, Vigillin, Merl, bacterial polynucleotide phosphorylase, ribosomal protein S3, and heat shock protein.

Additional exemplary formulations for oligonucleotides are described in U.S. Pat. Nos. 4,897,355; 4,394,448; 4,235,871; 4,231,877; 4,224,179; 4,753,788; 4,673,567; 4,247,411; 4,814,270; 5,567,434; 5,552,157; 5,565,213; 5,738,868; 5,795,587; 5,922,859; and 6,077,663, Int. App. Nos. PCT/US07/079203, filed Sep. 21, 2007; PCT/US07/080331, filed Oct. 3, 2007; U.S. patent application Ser. No. 12/123,922, filed May 28, 2008; U.S. Pat. Pub. Nos. 2006/0240093 and 2007/0135372 and U.S. Provisional App. Nos. 61/018,616, filed Jan. 2, 2008; 61/039,748, filed Mar. 26, 2008; 61/045,228, filed Apr. 15, 2008; 61/047,087, filed Apr. 22, 2008; 61/051,528, filed May 21, 2008; and 61/113,179 (filed Nov. 10, 2008), contents of which are herein incorporated by reference in their entirety. Behr (1994) Bioconjugate Chem. 5:382-389, and Lewis et al. (1996) PNAS 93:3176-3181), also describe formulations for oligonucleotides that are amenable to the invention, contents of which are herein incorporated by reference in their entirety.

A formulated oligonucleotide composition can assume a variety of states. In some examples, the composition can be at least partially crystalline, uniformly crystalline, and/or anhydrous (e.g., less than 80, 50, 30, 20, or 10% water). In another example, the oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a micro particle as can be appropriate for a crystalline composition). Generally, the oligonucleotide composition is formulated in a manner that is compatible with the intended method of administration.

In particular embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An oligonucleotide preparation can be formulated in combination with another agent, e.g., another therapeutic agent or an agent that stabilizes the oligonucleotide, e.g., a protein that complex with oligonucleotide to form an oligonucleotide-protein complex. Still other agents include chelators, e.g., EDTA (e.g., to remove divalent cations such as Mg²⁺), salts, DNAse inhibitors, RNAse inhibitors (e.g., a broad specificity RNAse inhibitor such as RNAsin) and so forth.

### Pharmaceutical Compositions

For administering to a subject, the anti-miR-130/301 agent or an expression vector encoding same can be formulated in pharmaceutically acceptable compositions. Thus, the disclosure also provides pharmaceutically acceptable compositions which comprise a therapeutically-effective amount of an anti-miR-130/301 agent or an expression encoding same, formulated together with one or more pharmaceutically acceptable carriers (additives) and/or diluents. As described in detail below, the pharmaceutical compositions of can be pecially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, agents can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Determination of a therapeutically effective amount is well within the capability of those skilled in the art. Generally, a therapeutically effective amount can vary with the subject's history, age, condition, sex, as well as the severity and type of the medical condition in the subject, and administration of other agents alleviate the disease or disorder to be treated.

Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The carrier can be in the form of a solid, semi-solid or liquid diluent, cream or a capsule.

The amount of oligonucleotide which can be combined with a carrier material to produce a single dosage form will generally be that amount of the compound which produces a therapeutic effect. Generally out of one hundred percent, this amount will range from about 0.1% to 99% of oligonucleotide, preferably from about 5% to about 70%, most preferably from 10% to about 30%.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. As used herein, the term ED denotes effective dose and is used in connection with animal models. The term EC denotes effective concentration and is used in connection with in vitro models.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized.

The therapeutically effective dose can be estimated initially from cell culture assays. A dose can be formulated in animal models to achieve a circulating plasma concentration range that includes the IC50 (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay.

The dosage can be determined by a physician and adjusted, as necessary, to suit observed effects of the treatment. Generally, the compositions are administered so that the agent is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 tmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In some embodiments, the compositions are administered at a dosage so that the agent has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered everyday or every third, fourth, fifth, or sixth day. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In some embodiments, the pharmaceutical composition further includes at least a second therapeutic agent (e.g., an agent other than anti-miR-130/301 agent). Exemplary therapeutic agents that can formulated with an anti-miR-130/301 agent include, but are not limited to, those found in Harrison's Principles of Internal Medicine, 17^{th} Edition, 2008, McGraw-Hill N.Y., N.Y.; Physicians Desk Reference, 63^{rd} Edition, 2008, Thomson Reuters, N.Y., N.Y.; Goodman & Gilman's The Pharmacological Basis of Therapeutics, 11^{th} Edition, 2005, McGraw-Hill N.Y., N.Y.; United States Pharmacopeia, The National Formulary, USP-32 NF-27, 2008, U.S. Pharmacopeia, Rockville, Md., the complete contents of all of which are incorporated herein by reference.

In some embodiments, the second therapeutic agent is an anti-hypertension agent or anti-hypertensive. In some embodiments, the second therapeutic agent is a therapeutic agent known in the art for treating fibrotic or fibroproliferative disease. In some embodiments, the second therapeutic agent is a therapeutic agent known in the art for inhibiting extracellualar matrix deposition or vascular stiffness.

### Combinations Therapy

In some embodiments, the anti-miR-130/301 agent can be administrated to a subject in combination with a pharmaceutically active agent, e.g., a second therapeutic agent. Exemplary pharmaceutically active compound include, but are not limited to, those found in Harrison's Principles of Internal Medicine, 13^{th} Edition, Eds. T. R. Harrison et al. McGraw-Hill N.Y., N.Y.; Physicians Desk Reference, 50^{th} Edition, 1997, Oradell N.J., Medical Economics Co.; Pharmacological Basis of Therapeutics, 8^{th} Edition, Goodman and Gilman, 1990; and United States Pharmacopeia, The National Formulary, USP XII NF XVII, 1990, the complete contents of all of which are incorporated herein by reference. The anti-miR-130/301 agent and the the second therapeutic agent can be administrated to the subject in the same pharmaceutical composition or in different pharmaceutical compositions (at the same time or at different times).

In some embodiments, the second therapeutic agent is an anti-hypertension agent or anti-hypertensive. Anti-hypertensives are a class of drugs that are used to treat hypertension (high blood pressure). Antihypertensive therapy seeks to prevent the complications of high blood pressure, such as stroke and myocardial infarction. Exemplary types of anti-hypertension agents include, but are not limited to, statins, diuretics, adrenergic receptor antagonists, calcium channel blockers, renin inhibitors, ACE inhibitors, angiotensin II receptor antagonists, aldosterone antagonists, vasodilators; alpha-2-agonists, and any combination thereof.

Exemplary anti-hypertension agents include, but are not limited to, bumetanide; ethacrynic acid; furosemide; torsemide; epitizide; hydrochlorothiazide; chlorothiazide; bendroflumethiazide; indapamide; chlorthalidone; metolazone; amiloride; triamterene; spironolactone; atenolol; metoprolol; nadolol; oxprenolol; pindolol; propranolol; timolol; doxazosin; phentolamine; indoramin; phenoxybenzamine; prazosin; terazosin; tolazoline; bucindolol; carvedilol; labetalol; amlodipine; felodipine; isradipine; lercanidipine; nicardipine; nifedipine; nimodipine; nitrendipine; diltiazem; verapamil; Aliskiren; captopril; enalapril; fosinopril; lisinopril; perindopril; quinapril; ramipril; trandolapril; benazepril; candesartan; eprosartan; irbesartan; losartan; olmesartan; telmisartan; valsartan; eplerenone; spironolactone; sodium nitroprusside; hydralazine; hydralazine derivatives; Clonidine; Guanabenz; Methyldopa; Moxonidine; Guanethidine; Reserpine; atorvastatin; fluvastatin; lovastatin; pitavastatin; pravastatin; rosuvastatin; simvastatin; and any combinations thereof.

In some embodiments, an anti-miR-103/301 agent can be administered in combination, or sequentially to (either before or after) administration of the anti-miR with a PDE-5 inhibitor, or other agent which increases endogenous cGMP, which is a secondary messenger for ANP. In some embodiments, a PDE-5 inhibitor is selected from the group consisting of: drug tadalafil (CIALIS™, ADCIRCA™), sildenafil (VIAGRA™), sildenafil citrate, zaprinast, LASSBio596, E-4010, and vardenafil, or a combination thereof. Examples of PDE5 inhibitors which can be used include, without limitation, pyrimidine and pyrimidinone derivatives, such as the compounds described in U.S. Pat. Nos. 6,677,335, 6,458,951, 6,251,904, 6,787,548, 5,294, 612, 5,250,534, 6,469,012, WO 94/28902, WO96/16657, EP0702555, and Eddahibi, Br. J. Pharmacol., 125(4): 681688 (1988); griseolic acid derivatives, such as the compounds disclosed in U.S. Pat. No. 4,460,765; 1-arylnaphthalene ligands, such as those described in Ukita, J. Med. Chem. 42(7): 1293-1305 (1999); quinazoline derivatives, such as 4-**3′,4′-(methylenedioxy)benzyl]amino]-6-methoxyquinazoline) and compounds described in U.S. Pat. Nos. 3,932,407, 4,146,718, and RE31,617; pyrroloquinolones and pyrrolopyridinones, such as those described in U.S. Pat. Nos. 6,686,349, 6,635,638, 6,818,646, US20050113402; carboline derivatives, such the compounds described in U.S. Pat. Nos. 6,492,358, 6,462,047, 6,821,975, 6,306,870, 6,117,881, 6,043,252, 3,819,631, US20030166641, WO 97/43287, Daugan et al, J Med. Chem, 46(21):4533-42 (2003), and Daugan et al, J Med. Chem, 9; 46(21):4525-32 (2003); imidazo derivatives, such as the compounds disclosed in U.S. Pat. Nos. 6,130,333, 6,566,360, 6,362,178, 6,582,351, US20050070541, and US20040067945; and compounds described in U.S. Pat. Nos. 6,825,197, 6,943,166, 5,981,527, 6,576,644, 5,859,009, 6,943,253, 6,864,253, 5,869,516, 5,488,055, 6,140,329, 5,859,006, 6,143,777, WO 96/16644, WO 01/19802, WO 96/26940, Dunn, Org. Proc. Res. Dev, 9: 88-97 (2005), and Bi et al, Bioorg Med Chem. Lett, 11(18):2461-4 (2001). Content of all of the above is incorporated herein by reference in its entirety.

Additional exemplary PDE5 inhibitors include, but are not limited to, zaprinast; MY-5445; dipyridamole; sulindac sulfone; vinpocetine; FR229934; 1-methyl-3-isobutyl-8-(methylamino)xanthine; furazlocillin; Sch-51866; E4021; GF-196960; IC-351; T-1032; sildenafil; tadalafil; vardenafil; DMPPO; RX-RA-69; KT-734; SKF-96231; ER-21355; BF/GP-385; NM-702; PLX650; PLX134; PLX369; PLX788; vesnarinone; sildenafil or a related compound disclosed in U.S. Pat. Nos. 5,346,901, 5,250,534, or 6,469,012; tadalafil or a related compound disclosed in U.S. Pat. Nos. 5,859,006, 6,140,329, 6,821,975, or 6,943,166; or vardenafil or a related compound disclosed in U.S. Pat. No. 6,362,178. Content of all of the above is incorporated herein by reference in its entirety.

In some embodiments, the second therapeutic agent is a therapeutic agent known in the art for treating fibrotic or fibroproliferative disease. In some embodiments, the second therapeutic agent is a therapeutic agent known in the art for inhibiting extracellualar matrix deposition or vascular stiffness Assays

The disclosure further provides an assay to determine if a subject is at risk of PH or PAH or a fibrotic or fibroproliferative disease. The assay comprising measuring or quantifying the amounts or expression of at least one (e.g., one, two, three, four or more) of microRNA-130a, microRNA-130b, microRNA-301a and microRNA-301b in a biological sample obtained from a subject; and comparing the measured or quantified amounts or level with a reference value, and if the amounts are increased relative to the reference value, identifying the subject as having an increased probability of having pulmonary hypertension or a fibrotic or fibroproliferative disease.

In some embodiments, the assay comprises detecting the level of at least one (e.g., one, two or three) of microRNA-130b, microRNA-301a and microRNA-301b.

In some embodiments, the assay comprises contacting a biological sample obtained from the subject with one or more probes to detect the levels of at least one (e.g., one, two, three, four or more) of microRNA-130a, microRNA-130b, microRNA-301a and microRNA-301b, wherein the level of at least one (e.g., one, two, three, four or more) of microRNA-130a, microRNA-130b, microRNA-301a or microRNA-301b above a predefined reference level identifies the subject predicted to be at risk of pulmonary hypertension or PAH or a fibrotic or fibroproliferative disease.

In some embodiments, the biological sample is a tissue samples, such as blood sample, e.g., plasma sample. In some embodiments of the methods, systems and assays as disclosed herein miR-130/301 levels can be determined by any methods known by persons of ordinary skill in the art. For example, miR-130/301 levels can be determined using a nucleic acid probe in, for example, Northern blot analysis, PCR, RT-PCR, quantitative RT-PCR, or other methods to determine expression levels of nucleic acids in a biological sample. Any means to analyze a miRNA expression profile known by persons of ordinary skill in the art can be used in the methods, systems and assays as disclosed herein, for example by microarray assay.

Another aspect of the present invention relates to a method, system and assay to identify subjects amenable to treatment with an anti-miR-130/301 agent as disclosed herein. In one embodiment, the present invention provides for an assay which enables one to measure, or quantify, the amount of at least one member (e.g, one, two, three, four or more members) of miR-130/301 family in a biological sample, e.g., a blood or plasma sample, obtained from a subject; and compare the measured, or quantified amount with a reference value, and if the amount of the member is increased relative to the reference value, the subject is identified as having an increased probability of having, or at risk of having PH, PAH, fibrotic or or fibroproliferative disease disease. In such embodiments, the subject can be administered an anti-miR-130/301 agent as disclosed herein. In some embodiments, subjects with normal or high levels (e.g., above a predefined reference value) of miR-130/301 are amenable to treatment with an anti-miR-130/301 agent as disclosed herein.

Accordingly, one aspect of the present invention relates to an assay comprising: (a) contacting a biological sample obtained from a subject with a detectable antibody specific for miR-130/310 family member or detectable nucleic acid complementary to at least part of miR-130/301 family member; (b) washing the sample to remove unbound antibody or unbound nucleic acid; (c) measuring the intensity of the signal from the bound, detectable antibody or bound detectable nucleic acid; (d) comparing the measured intensity of the signal with a reference value and if the measured intensity is normal and/or increased relative to the reference value; the subject is identified as having an increased probability of having PH, PAH, fibrotic or fibroproliferative disease.

In some embodiments, the reference value is the signal of bound antibody or bound nucleic acid in a sample from a healthy subject, i.e., a subject who does not have PH, PAH, fibrotic or fibroproliferative disease.

In some embodiments, the subject is recommended a treatment with an anti-miR-130/301 agent where the content from the display module produces a signal indicative of the subject having an increased probability of having PH, PAH, fibrotic or fibroproliferative disease.

In some embodiments, a subject is not recommended for treatment with an anti-miR-130/301 agent where the content from the display module produces a signal indicative of the subject having an increased probability of having PH, PAH, fibrotic or fibroproliferative disease.

In some embodiments, a predetermined level of miR-130/301 family member is the level of miR-130/301 family member expression from a subject, or a representative pool of healthy subjects. In some embodiments, a pre-determined standard of miR-130/301 family member is the level of miR-130/301 family member expression from a subject or a representative pool or cohort of healthy subjects.

Another aspect of the present invention relates to a method, system and assay to identify subjects amenable to treatment with an anti-miR-130/301 agent as disclosed herein. In one embodiment, the present invention provides for an assay which enables one to measure, or quantify, the amount of one or more (e.g., one, two, three, four or more) of miR-130a, miR-130b, miR-301a and miR-301b in a biological sample, e.g., a blood or plasma sample, obtained from a subject; and compare the measured, or quantified amount, of one or more (e.g., one, two, three, four or more) of miR-130a, miR-130b, miR-301a and miR-301b with a reference value, and if the amount of one or more (e.g., one, two, three, four or more) of miR-130a, miR-130b, miR-301a and miR-301b is increased relative to the reference value, the subject is identified as having an increased probability of having, or at risk of having high blood pressure, hypertension or cardiovascular disease. In such embodiments, the subject can be administered an anti-miR-130/301 agent as disclosed herein. In some embodiments, subjects with normal or high levels (e.g., above a predefined reference value) of one or more (e.g., one, two, three, four or more) of miR-130a, miR-130b, miR-301a and miR-301b are amenable to treatment with an anti-miR-130/301 agent as disclosed herein, or where it is desirable to increase ANP levels in the subject.

Accordingly, one aspect of the present invention relates to an assay comprising: (a) contacting a biological sample obtained from a subject with a detectable antibody specific for or detectable nucleic acid complementary to at least part of one or more (e.g., one, two, three, four or more) of miR-130a, miR-130b, miR-301a and miR-301b; (b) washing the sample to remove unbound antibody or unbound nucleic acid; (c) measuring the intensity of the signal from the bound, detectable antibody or bound detectable nucleic acid; (d) comparing the measured intensity of the signal with a reference value and if the measured intensity is normal and/or increased relative to the reference value; the subject is identified as having an increased probability of having PH, PAH, fibrotic or fibroproliferative disease.

Another aspect of the present invention relates to a system for obtaining data from at least one test sample obtained from at least one subject, the system comprising: (a) a determination module configured to receive said at least one test sample and perform at least one analysis on said at least one test sample to determine the level of expression of one or more (e.g., one, two, three, four or more) of miR-130a, miR-130b, miR-301a and miR-301b; (b) a storage device configured to store data output from said determination module; and (c) a display module for displaying a content based in part on the data output from said determination module, wherein the content comprises a signal indicative of the presence of at least one of these conditions determined by the determination module, or a signal indicative of the absence of at least one of these conditions determined by the determination module. In some embodiments, the level of miRNA is determined with the levels of one or more other biomarkers.

In some embodiments, the content displayed from the display module of the system as disclosed herein can further comprise a signal indicative of the subject being recommended to receive a particular treatment regimen, for example, if the subject has increased expression or level of one or more (e.g., one, two, three, four or more) of miR-130a, miR-130b, miR-301a and miR-301b, a signal is produced to recommend the subject be administered a composition comprising an anti-miR-130/301 agent as disclosed herein.

In some embodiments, the subject is recommended a treatment with a composition comprising an anti-miR-103/301 agent where the content from the display module produces a signal indicative of increased level or expression miR-130a. In some embodiments, the subject is recommended a treatment with a composition comprising an anti-miR-103/301 agent where the content from the display module produces a signal indicative of increased level or expression miR-130b. In some embodiments, the subject is recommended a treatment with a composition comprising an anti-miR-103/301 agent where the content from the display module produces a signal indicative of increased level or expression miR-301a. In some embodiments, the subject is recommended a treatment with a composition comprising an anti-miR-103/301 agent where the content from the display module produces a signal indicative of increased level or expression miR-301b.

In some embodiments, the subject is recommended a treatment with a composition comprising an anti-miR-103/301 agent where the content from the display module produces a signal indicative of increased level or expression at least two of miR-130a, miR-130b, miR-130b, miR-301a and miR-301b. For example increased level or expression of miR-130a and miR-130b, miR-130a and miR-301a, miR-130a and miR-301b, miR-130b and miR-301a, miR-130b and miR-301b, or miR-301a and miR-301b.

In some embodiments, the subject is recommended a treatment with a composition comprising an anti-miR-103/301 agent where the content from the display module produces a signal indicative of increased level or expression at least three of miR-130a, miR-130b, miR-130b, miR-301a and miR-301b. For example increased level or expression of miR-130a, miR-130b and miR-301a; miR-130a, miR-130b and miR-301b; miR-130a, miR-301a and miR-301b; or miR-130b, miR-301a and miR-301b.

In some embodiments, the subject is recommended a treatment with a composition comprising an anti-miR-103/301 agent where the content from the display module produces a signal indicative of increased level or expression all four of miR-130a, miR-130b, miR-130b, miR-301a and miR-301b.

In some embodiments, the methods and assay can be carried out in an automated and/or high-throughput system. One aspect of the present invention relates to a computerized system for processing the assays as disclosed herein and identifying the level of expression of one or more members of miR-130/301 family, for example expression level of one or more (e.g., one, two, three, four or more) of miR-130a, miR-130b, miR-301a and miR-301b.

In some embodiments, a computer system can include: (a) at least one memory containing at least one computer program adapted to control the operation of the computer system to implement a method that includes: (i) receiving data of the level of expression or intensity of signal of measured mRNA of one or more (e.g., one, two, three, four or more) of miR-130a, miR-130b, miR-301a and miR-301b, (ii) generating a report of intensity of expression or intensity of signal of measured one or more (e.g., one, two, three, four or more) of miR-130a, miR-130b, miR-301a and miR-301b mRNA in a biological sample and optionally a reference level for one or more (e.g., one, two, three, four or more) of miR-130a, miR-130b, miR-301a and miR-301b mRNA signal intensity; and (b) at least one processor for executing the computer program.

In some embodiments, a computer system can include: (a) at least one memory containing at least one computer program adapted to control the operation of the computer system to implement a method that includes: (i) receiving data of the level of expression or intensity of signal of measured one or more (e.g., one, two, three, four or more) of miR-130a, miR-130b, miR-301a and miR-301b levels (ii) generating a report of intensity of expression or intensity of signal of measured one or more (e.g., one, two, three, four or more) of miR-130a, miR-130b, miR-301a and miR-301b levels in a biological sample and optionally a reference level for one or more (e.g., one, two, three, four or more) of miR-130a, miR-130b, miR-301a and miR-301b level signal intensity; and (b) at least one processor for executing the computer program.

The computer system can include one or more general or special purpose processors and associated memory, including volatile and non-volatile memory devices. The computer system memory can store software or computer programs for controlling the operation of the computer system to make a special purpose computer system according to the invention or to implement a system to perform the methods and analysis according to the invention.

In some embodiments, a computer system can include, for example, an Intel or AMD x86 based single or multi-core central processing unit (CPU), an ARM processor or similar computer processor for processing the data. The CPU or microprocessor can be any conventional general purpose single- or multi-chip microprocessor such as an Intel and AMD processor, a SPARC processor, or an ARM processor. In addition, the microprocessor may be any conventional or special purpose microprocessor such as a digital signal processor or a graphics processor. The microprocessor typically has conventional address lines, conventional data lines, and one or more conventional control lines. As described below, the software according to the invention can be executed on dedicated system or on a general purpose computer having a DOS, CPM, Windows, Unix, Linix or other operating system. The system can include non-volatile memory, such as disk memory and solid state memory for storing computer programs, software and data and volatile memory, such as high speed ram for executing programs and software.

Computer-readable physical storage media useful in various embodiments of the invention can include any physical computer-readable storage medium, e.g., solid state memory (such as flash memory), magnetic and optical computer-readable storage media and devices, and memory that uses other persistent storage technologies. In some embodiments, a computer readable media can be any tangible media that allows computer programs and data to be accessed by a computer. Computer readable media can include volatile and nonvolatile, removable and non-removable tangible media implemented in any method or technology capable of storing information such as computer readable instructions, program modules, programs, data, data structures, and database information. In some embodiments of the invention, computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks), Blue-ray, USB drives, micro-SD drives, or other optical storage media, magnetic cassettes, magnetic tape, magnetic disk storage or other magnetic storage media, other types of volatile and non-volatile memory, and any other tangible medium which can be used to store information and which can read by a computer including and any suitable combination of the foregoing.

The present invention can be implemented on a stand-alone computer or as part of a networked computer system. In a stand-alone computer, all the software and data can reside on local memory devices, for example an optical disk or flash memory device can be used to store the computer software for implementing the invention as well as the data. In alternative embodiments, the software or the data or both can be accessed through a network connection to remote devices. In one embodiment, the invention can use a client-server environment over a network, e.g., a public network such as the internet or a private network to connect to data and resources stored in remote and/or centrally located locations. In this embodiment, a server such as a web server can provide access, either open access, pay as you go or subscription based access to the information provided according to the invention. In a client server environment, a client computer executing a client software or program, such as a web browser, connects to the server over the network. The client software provides a user interface for a user of the invention to input data and information and receive access to data and information. The client software can be viewed on a local computer display or other output device and can allow the user to input information, such as by using a computer keyboard, mouse or other input device. The server executes one or more computer programs that receives data input through the client software, processes data according to the invention and outputs data to the user, as well as provide access to local and remote computer resources. For example, the user interface can include a graphical user interface comprising an access element, such as a text box, that permits entry of data from the assay, e.g., the data from a positive reference cancer cell, as well as a display element that can provide a graphical read out of the results of a comparison with a cancer cell with a known metastatic potential or invasive capacity, or data sets transmitted to or made available by a processor following execution of the instructions encoded on a computer-readable medium.

Embodiments of the invention also provide for systems (and computer readable medium providing instructions for causing computer systems) to perform a method for determining quality assurance of a pluripotent stem cell population according to the methods as disclosed herein.

In some embodiments of the invention, the computer system software can include one or more functional modules, which can be defined by computer executable instructions recorded on computer readable media and which cause a computer to perform, when executed, a method according to one or more embodiments of the invention. The modules can be segregated by function for the sake of clarity, however, it should be understood that the modules need not correspond to discreet blocks of code and the described functions can be carried out by the execution of various software code portions stored on various media and executed at various times. Furthermore, it should be appreciated that the modules can perform other functions, thus the modules are not limited to having any particular function or set of functions. In some embodiments, functional modules are, for example, but are not limited to, an array module, a determination module, a storage module, a reference comparison module, a normalization module, and a display module to display the results (e.g., the invasive potential of the test cancer cell population). The functional modules can be executed using one or multiple computers, and by using one or multiple computer networks.

The information embodied on one or more computer-readable media can include data, computer software or programs, and program instructions that, as a result of being executed by a computer, transform the computer to special purpose machine and can cause the computer to perform one or more of the functions described herein. Such instructions can be originally written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied can reside on one or more of the components of a computer system or a network of computer systems according to the invention.

In some embodiments, a computer-readable media can be transportable such that the instructions stored thereon can be loaded onto any computer resource to implement the aspects of the present invention discussed herein. In addition, it should be appreciated that the instructions stored on computer readable media are not limited to instructions embodied as part of an application program running on a host computer. Rather, the instructions may be embodied as any type of computer code (e.g., object code, software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

In some embodiments, a system as disclosed herein, can receive data of intensity of expression of one or more (e.g., one, two, three, four or more) of miR-130a, miR-130b, miR-301a and miR-301b expression from any method of determining the level of expression. Where the quantity to be measured is protein expression, the system as disclosed herein can be configured to receive data from an automated protein analysis systems, for example, using immunoassay, for example western blot analysis or ELISA, or a high through-put protein detection method, for example but are not limited to automated immunohistochemistry apparatus, for example, robotically automated immunodetection apparatus which in an automated system can perform immunohistochemistry procedure and detect intensity of immunostaining, such as intensity of an antibody staining of the substrates and produce output data. Examples of such automated immunohistochemistry apparatus are commercially available, and can be readily adapted to automatically detect the level of protein expression in the assay as disclosed herein, and include, for example but not limited to such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx6000™, for Drug Discovery labs. Both systems BioGenex systems perform “All-in-One, All-at-Once” functions for cell and tissue testing, such as Immunohistochemistry (IHC) and In Situ Hybridization (ISH).

In some embodiments, a system as disclosed herein, can receive data of intensity of protein expression of NT-proANP from an automated ELISA system (e.g. DSX® or DK® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters), or adapted systems thereof for detecting cells on the separated substrates as disclosed herein.

In some embodiments, a system as disclosed herein, can receive data can receive data of intensity of mRNA expression of one or more of miR-130/301 family members from any method of determining gene or nucleic acid expression. In some embodiments, the system as disclosed herein can be configured to receive data from an automated gene expression analysis system, e.g., an automated protein expression analysis including but not limited Mass Spectrometry systems including MALDI-TOF, or Matrix Assisted Laser Desorption Ionization—Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array.

In some embodiments of the present invention, an automated gene expression analysis system can record the data electronically or digitally, annotated and retrieved from databases including, but not limited to GenBank (NCBI) protein and DNA databases such as genome, ESTs, SNPS, Traces, Celara, Ventor Reads, Watson reads, HGTS, etc.; Swiss Institute of Bioinformatics databases, such as ENZYME, PROSITE, SWISS-2DPAGE, Swiss-Prot and TrEMBL databases; the Melanie software package or the ExPASy WWW server, etc., the SWISS-MODEL, Swiss-Shop and other network-based computational tools; the Comprehensive Microbial Resource database (The institute of Genomic Research). The resulting information can be stored in a relational data base that may be employed to determine homologies between the reference data or genes or proteins within and among genomes.

In some embodiments, a system as disclosed herein, can receive data can receive data from an allele-specific PCR. The term “allele-specific PCR” refers to PCR techniques where the primer pairs are chosen such that amplification is dependent upon the input template nucleic acid containing the polymorphism of interest. In such embodiments, primer pairs are chosen such that at least one primer is an allele-specific oligonucleotide primer. In some sub-embodiments of the present invention, allele-specific primers are chosen so that amplification creates a restriction site, facilitating identification of a polymorphic site. In other embodiments of the present invention, amplification of the target polynucleotide is by multiplex PCR (Wallace et al. (PCT Application WO89/10414)). Through the use of multiplex PCR, a multiplicity of regions of a target polynucleotide can be amplified simultaneously. This is particularly advantageous in embodiments where more than one SNP is to be detected.

In another embodiment, multiplex PCR procedures using allele-specific primers can be used to simultaneously amplify multiple regions of a target nucleic acid (PCT Application WO89/10414), enabling amplification only if a particular allele is present in a sample. Other embodiments using alternative primer-guided nucleotide incorporation procedures for assaying polymorphic sites in DNA can be used, and have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Nat. Acad. Sci. (U.S.A.) 88:1143-1147 (1991); Bajaj et al. (U.S. Pat. No. 5,846,710); Prezant, T. R. et al., Hum Mutat. 1: 159-164 (1992); Ugozzoli, L. et al., GATA 9:107-112 47 (1992); Nyr6n, P. et al., Anal. Biochem. 208:171-175 (1993)).

Other known nucleic acid amplification procedures include transcription-based amplification systems (Malek, L. T. et al., U.S. Pat. No. 5,130,238; Davey, C. et al., European Patent Application 329,822; Schuster et al.) U.S. Pat. No. 5,169,766; Miller, H. I. et al., PCT—Application WO89/06700; Kwoh, D. et al., Proc. Natl. Acad. Sci. (U.S.A.) 86:1173 Z1989); Gingeras, T. R. et al., PCT Application WO88/10315)), or isothermal amplification methods (Walker, G. T. et al., Proc. Natl. 4cad Sci. (U.S.A) 89:392-396 (1992)) can also be used.

In some embodiments, a system as disclosed herein, can receive data from any genotyping assay known by persons of ordinary skill in the art, including, but not limited to, those disclosed in U.S. Pat. No. 6,472,157; U.S. Patent Application Publications 20020016293, 20030099960, 20040203034; WO 0180896, all of which are hereby incorporated by reference, or by linkage disequlibrium, restriction fragment length polymorphism” (RFLP) analysis, single strand conformational polymorphism (SSCP), RNaseI for mismatch detection, SNP mapping (Davis et al, Methods Mol Biology, 2006; 351; 75-92); Nanogen Nano Chip, (keen-Kim et al, 2006; Expert Rev Mol Diagnostic, 6; 287-294); Rolling circle amplification (RCA) combined with circularable oligonucleotide probes (c-probes) for the detection of nucleic acids (Zhang et al, 2006: 363; 61-70), luminex XMAP system for detecting multiple SNPs in a single reaction vessel (Dunbar S A, Clin Chim Acta, 2006; 363; 71-82; Dunbar et al, Methods Mol Med, 2005; 114:147-1471), enzymatic mutation detection methods (Yeung et al, Biotechniques, 2005; 38; 749-758), matrix-assisted laser desorption ionization/time-of-flight (MALDI-TOF) mass spectrometric (MS) analysis, long-range PCR (LR-PCR), genotype assays disclosed in Kwok, Hum Mut 2002; 9; 315-323 and Kwok, Annu Rev Genomic Hum Genetics, 2001; 2; 235-58, (which are incorporated herein in their entirety by reference), INVADER® Assay (Gut et al, Hum Mutat, 2001; 17:475-92, Shi et al, Clin Chem, 2001, 47, 164-92, and Olivier et al, Mutat Res, 2005; 573:103-110), the method utilizing FLAP endonucleases (U.S. Pat. No. 6,706,476) and the SNPlex genoptyping systems (Tobler et al, J. Biomol Tech, 2005; 16; 398-406) and other such genotyping assays known to one of ordinary skill in the art.

Examples of suitable connection technologies for use with the present invention include, for example parallel interfaces (e.g., PATA), serial interfaces (e.g., SATA, USB, Firewire,), local area networks (LAN), wide area networks (WAN), Internet, Intranet, and Extranet, and wireless (e.g., Blue Tooth, Zigbee, WiFi, WiMAX, 3G, 4G) communication technologies

Storage devices are also commonly referred to in the art as “computer-readable physical storage media” which is useful in various embodiments, and can include any physical computer-readable storage medium, e.g., magnetic and optical computer-readable storage media, among others. Carrier waves and other signal-based storage or transmission media are not included within the scope of storage devices or physical computer-readable storage media encompassed by the term and useful according to the invention. The storage device is adapted or configured for having recorded thereon cytokine level information. Such information can be provided in digital form that can be transmitted and read electronically, e.g., via the Internet, on diskette, via USB (universal serial bus) or via any other suitable mode of communication.

As used herein, “stored” refers to a process for recording information, e.g., data, programs and instructions, on the storage device that can be read back at a later time. Those skilled in the art can readily adopt any of the presently known methods for recording information on known media to contribute to the data of (i) the level of expression of one or more (e.g., one, two, three, four or more) of miR-130a, miR-130b, miR-301a and miR-301b mRNA or (ii) expression level of one or more (e.g., one, two, three, four or more) of miR-130a, miR-130b, miR-301a and miR-301; and generate a report of the presence or absence, or amount of (i) the expression of one or more (e.g., one, two, three, four or more) of miR-130a, miR-130b, miR-301a and miR-301b mRNA or (ii) expression level of one or more (e.g., one, two, three, four or more) of miR-130a, miR-130b, miR-301a and miR-301b.

A variety of software programs and formats can be used to store information on the storage device. Any number of data processor structuring formats (e.g., text file or database) can be employed to obtain or create a medium having recorded scorecard thereon.

In some embodiment, the system has a processor for running one or more programs, e.g., where the programs can include an operating system (e.g., UNIX, Windows), a relational database management system, an application program, and a World Wide Web server program. The application program can be a World Wide Web application that includes the executable code necessary for generation of database language statements (e.g., Structured Query Language (SQL) statements). The executables can include embedded SQL statements. In addition, the World Wide Web application can include a configuration file which contains pointers and addresses to the various software entities that provide the World Wide Web server functions as well as the various external and internal databases which can be accessed to service user requests. The Configuration file can also direct requests for server resources to the appropriate hardware devices, as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the system as disclosed herein can be used to compare the data of intensity of one or more of (i) the level of expression of one or more (e.g., one, two, three, four or more) of miR-130a, miR-130b, miR-301a and miR-301b mRNA or (ii) expression level of one or more (e.g., one, two, three, four or more) of miR-130a, miR-130b, miR-301a and miR-301b; and generate a report of the presence or absence, or amount of (i) the expression of one or more (e.g., one, two, three, four or more) of miR-130a, miR-130b, miR-301a and miR-301b mRNA or (ii) expression of one or more (e.g., one, two, three, four or more) of miR-130a, miR-130b, miR-301a and miR-301b.

In some embodiments of this aspect and all other aspects of the present invention, the system can compare the data in a “comparison module” which can use a variety of available software programs and formats for the comparison operative to compare sequence information determined in the determination module to reference data. In one embodiment, the comparison module is configured to use pattern recognition techniques to compare levels of expression (e.g., mRNA levels and/or protein levels) as well as compare sequence information from one or more entries to one or more reference data patterns. The comparison module may be configured using existing commercially-available or freely-available software for comparing patterns, and may be optimized for particular data comparisons that are conducted. The comparison module can also provide computer readable information related to the level or amount of intensity of expression of the level of expression of one or more (e.g., one, two, three, four or more) of miR-130a, miR-130b, miR-301a and miR-301b mRNA; or expression level of one or more (e.g., one, two, three, four or more) of miR-130a, miR-130b, miR-301a and miR-301b and the like as disclosed herein.

By providing data of the intensity of expression of mRNA or expression level of miRNA in computer-readable form, one can use the data to compare with data within the storage device. For example, search programs can be used to identify relevant reference data (i.e. data of appropriate reference cancer cell lines) that match the same type of cancer as the cancer of the test cancer cell population. The comparison made in computer-readable form provides computer readable content which can be processed by a variety of means. The content can be retrieved from the comparison module, the retrieved content.

In some embodiments, the comparison module provides computer readable comparison result that can be processed in computer readable form by predefined criteria, or criteria defined by a user, to provide a report which comprises content based in part on the comparison result that may be stored and output as requested by a user using a display module. In some embodiments, a display module enables display of a content based in part on the comparison result for the user, wherein the content is a report indicative of the results of the comparison of the intensity of expression of (i) the level of expression of one or more (e.g., one, two, three, four or more) of miR-130a, miR-130b, miR-301a and miR-301b mRNA; and/or (ii) expression level of one or more (e.g., one, two, three, four or more) of miR-130a, miR-130b, miR-301a and miR-301b.

In some embodiments of this aspect and all other aspects of the present invention, the comparison module, or any other module of the invention, can include an operating system (e.g., UNIX, Windows) on which runs a relational database management system, a World Wide Web application, and a World Wide Web server. World Wide Web application can includes the executable code necessary for generation of database language statements [e.g., Standard Query Language (SQL) statements]. The executables can include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using an HTML interface provided by Web browsers and Web servers. In other embodiments of the invention, other interfaces, such as HTTP, FTP, SSH and VPN based interfaces can be used to connect to the Internet databases.

In some embodiments of this aspect and all other aspects of the present invention, a computer-readable media can be transportable such that the instructions stored thereon, such as computer programs and software, can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement aspects of the present invention. The computer executable instructions can be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The computer instructions can be implemented in software, firmware or hardware and include any type of programmed step undertaken by modules of the information processing system. The computer system can be connected to a local area network (LAN) or a wide area network (WAN). One example of the local area network can be a corporate computing network, including access to the Internet, to which computers and computing devices comprising the data processing system are connected. In one embodiment, the LAN uses the industry standard Transmission Control Protocol/Internet Protocol (TCP/IP) network protocols for communication. Transmission Control Protocol Transmission Control Protocol (TCP) can be used as a transport layer protocol to provide a reliable, connection-oriented, transport layer link among computer systems. The network layer provides services to the transport layer. Using a two-way handshaking scheme, TCP provides the mechanism for establishing, maintaining, and terminating logical connections among computer systems. TCP transport layer uses IP as its network layer protocol. Additionally, TCP provides protocol ports to distinguish multiple programs executing on a single device by including the destination and source port number with each message. TCP performs functions such as transmission of byte streams, data flow definitions, data acknowledgments, lost or corrupt data re-transmissions, and multiplexing multiple connections through a single network connection. Finally, TCP is responsible for encapsulating information into a datagram structure. In alternative embodiments, the LAN can conform to other network standards, including, but not limited to, the International Standards Organization's Open Systems Interconnection, IBM's SNA, Novell's Netware, and Banyan VINES.

In some embodiments, the computer system as described herein can include any type of electronically connected group of computers including, for instance, the following networks: Internet, Intranet, Local Area Networks (LAN) or Wide Area Networks (WAN). In addition, the connectivity to the network may be, for example, remote modem, Ethernet (IEEE 802.3), Token Ring (IEEE 802.5), Fiber Distributed Datalink Interface (FDDI) or Asynchronous Transfer Mode (ATM). The computing devices can be desktop devices, servers, portable computers, hand-held computing devices, smart phones, set-top devices, or any other desired type or configuration. As used herein, a network includes one or more of the following, including a public internet, a private internet, a secure internet, a private network, a public network, a value-added network, an intranet, an extranet and combinations of the foregoing.

In some embodiments of this aspect and all other aspects of the present invention, a comparison module provides computer readable data that can be processed in computer readable form by predefined criteria, or criteria defined by a user, to provide a retrieved content that may be stored and output as requested by a user using a display module.

In accordance with some embodiments of the invention, the computerized system can include or be operatively connected to an output module. In some embodiments, the output module is a display module, such as computer monitor, touch screen or video display system. The display module allows user instructions to be presented to the user of the system, to view inputs to the system and for the system to display the results to the user as part of a user interface. Optionally, the computerized system can include or be operative connected to a printing device for producing printed copies of information output by the system.

In some embodiments, the results can be displayed on a display module or printed in a report, e.g., a to indicate any one or more of (i) the level of expression of one or more (e.g., one, two, three, four or more) of miR-130a, miR-130b, miR-301a and miR-301b mRNA; and/or (ii) expression level of one or more (e.g., one, two, three, four or more) of miR-130a, miR-130b, miR-301a and miR-301b or any other report envisioned by the end user.

In some embodiments, the report is a hard copy printed from a printer. In alternative embodiments, the computerized system can use light or sound to report the result. In some embodiments, the report can also present text, either verbally or written, giving a recommendation of if a subject is amenable to treatment with an anti-miR-130/301 agent as disclosed herein. In other embodiments, the report provides just values or numerical scores for the presence of any one or more of the (i) the level of expression of one or more (e.g., one, two, three, four or more) of miR-130a, miR-130b, miR-301a and miR-301b mRNA; and/or (ii) expression level of one or more (e.g., one, two, three, four or more) of miR-130a, miR-130b, miR-301a and miR-301b.

In some embodiments of this aspect and all other aspects of the present invention, the report data from the comparison module can be displayed on a computer monitor as one or more pages of the printed report. In one embodiment of the invention, a page of the retrieved content can be displayed through printable media. The display module can be any device or system adapted for display of computer readable information to a user. The display module can include speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc.

In some embodiments of the present invention, a World Wide Web browser can be used to provide a user interface to allow the user to interact with the system to input information, construct requests and to display retrieved content. In addition, the various functional modules of the system can be adapted to use a web browser to provide a user interface. Using a Web browser, a user can construct requests for retrieving data from data sources, such as data bases and interact with the comparison module to perform comparisons and pattern matching. The user can point to and click on user interface elements such as buttons, pull down menus, scroll bars, etc. conventionally employed in graphical user interfaces to interact with the system and cause the system to perform the methods of the invention. The requests formulated with the user's Web browser can be transmitted over a network to a Web application that can process or format the request to produce a query of one or more database that can be employed to provide the pertinent information related to the tumor type, the retrieved content, process this information and output the results, e.g. at least one of any of the following: % invasion, % invasion under specific conditions (e.g., culture time, presence of drugs, tumor of different geometry, e.g., with or without hypoxic cells). In some embodiments, these values or their combination can exhibit strong correlation with invasive capacity of cells in the patients. In some embodiments, output information of the % invasion, % invasion under specific conditions (e.g., culture time, presence of drugs, tumor of different geometry, e.g., with or without hypoxic cells) can vary with different tumor types, and can be determined by one of ordinary skill in the art by comparing the numbers across a range of highly metastatic cancer cell lines as disclosed herein.

### Kits

In another aspect, the disclosure provides kits for the practice of the methods of this invention. The kits preferably include one or more containers containing an anti-miR-130/301 agent and a pharmaceutically acceptable excipient. The kit can optionally contain additional therapeutics to be co-administered with the anti-miR-130/301 agent. The kit can comprise instructions for administration of an anti-miR-130/301 agent to a subject with a PH, PAH or fibrotic disease or disorder. The kits can also optionally include appropriate systems (e.g. opaque containers) or stabilizers (e.g. antioxidants) to prevent degradation of the anti-miR-130/301 agent by light or other adverse conditions.

The kits can optionally include instructional materials containing directions (i.e., protocols) providing for the use of anti-miR-130/301 agents for the treatment of PH, PAH, fibrotic or fibroproliferative disease disease in a mammal. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media can include addresses to internet sites that provide such instructional materials.

Exemplary embodiments of the various aspects disclosed herein can be described by one or more of the following numbered paragraphs:
1. A method of inhibiting, preventing or treating pulmonary hypertension (PH) or pulmonary arterial hypertension (PAH) or a symptom thereof in a subject in need thereof, comprising inhibiting activity or expression of at least one of (e.g., one, two, three, four, or five of) microRNA-130a, microRNA-130b, microRNA-301a, microRNA-301b, and microRNA-454.
2. A method for inhibiting, preventing or treating a fibrotic or fibroproliferative disease or a symptom thereof in a subject in need thereof, comprising inhibiting activity or expression of at least one of (e.g., one, two, three, four, or five of) microRNA-130a, microRNA-130b, microRNA-301a, microRNA-301b, and microRNA-454.
3. The method of paragraphs 2, wherein the fibrotic or fibroproliferative disease is cancer/metastasis.
4. A method of modulating extracellular matrix deposition or vascular/tissue stiffness in a subject in need thereof, comprising inhibiting activity or expression of at least one of (e.g., one, two, three, four, or five of) microRNA-130a, microRNA-130b, microRNA-301a, microRNA-301b, and microRNA-454.
5. The method of paragraph 4, wherein said modulating matrix deposition or vascular/tissue stiffness is inhibiting or reducing extracellular matrix deposition or vascular/tissue stiffness.
6. The method of any of paragraphs 1-5, wherein the method comprises administering to the subject an effective amount of an inhibitor of that inhibits the activity of at least two of (e.g., two, three, four, or five of) microRNA-130a, microRNA-130b, microRNA-301a, microRNA-301b, and microRNA-454.
7. The method of any of paragraphs 1-6, wherein the method comprises administering to the subject an effective amount of an inhibitor of that inhibits the activity of at least three of (e.g., two, three, four of) microRNA-130a, microRNA-130b, microRNA-301a, microRNA-301b, and microRNA-454.
8. The method of any of paragraphs 1-7, wherein the method comprises administering to the subject an effective amount of an inhibitor of that inhibits the activity of at least four of (e.g., two, three, four of) microRNA-130a, microRNA-130b, microRNA-301a, microRNA-301b, and microRNA-454.
9. The method of any of paragraphs 1-8, wherein the method comprises inhibiting the activity of microRNA-130a, microRNA-130b, microRNA-301a, and microRNA-301b.
10. The method of any of paragraphs 1-9, wherein the method comprises inhibiting the activity of microRNA-130a, microRNA-130b, microRNA-301a, microRNA-301b, and microRNA-454.
11. The method of any of paragraphs 1-10, wherein the inhibitor is selected from the group consisting of small molecules, nucleic acids, nucleic acid analogues, peptides, proteins, antibodies, antigen binding fragments of antibodies, and any combinations thereof.
12. The method of any of paragraphs 1-11, wherein the inhibitor is an oligonucleotide.
13. The method of any of paragraphs 1-12, wherein the inhibitor is an anti-miR, antagomir, antisense oligonucleotide, ribozyme, siRNA, or shRNA.
14. The method of any of paragraphs 1-13, wherein the inhibitor comprise a nucleotide sequence that is substantially complementary to at least a portion of nucleic acid sequence selected from the group consisting of: human miR-130a (SEQ ID NO: 20); human miR-130b (SEQ ID NO: 21); human miR-301a (SEQ ID NO: 22); human miR-301b (SEQ ID NO: 23); mouse miR-130a (SEQ ID NO: 24); mouse miR-130b (SEQ ID NO: 25); miR-454 (SEQ ID NO: 27); and any combinations thereof.
15. The method of any of paragraphs 1-14, wherein the inhibitor comprises a nucleotide sequence that is substantially complementary to at least a portion of nucleic acid sequence selected from the group consisting of has-miR-130a-3p (cagugcaauguuaaaagggcau) (SEQ ID NO: 4), has-miR-130b-3p (cagugcaaugaugaaagggcau) (SEQ ID NO: 5), has-miR-301a-3p (cagugcaauaguauugucaaagc) (SEQ ID NO: 6), has-miR-301b-3p (cagugcaaugauauugucaaagc) (SEQ ID NO: 7), has-miR-454-3p (uagugcaauauugcuuauagggu) (SEQ ID NO: 26), and any combinations thereof.
16. The method of any of paragraphs 1-15, wherein the inhibitor comprises the nucleotide sequence 5′-TTGCACT-3′ (SEQ ID NO: 2) or 5′-ATTGCACT-3′ (SEQ ID NO: 3)
17. The method of any of paragraphs 1-16, wherein the inhibitor inhibits the activity of microRNA-130a, microRNA-130b, microRNA-301a, microRNA-301b, and microRNA-454.
18. The method of any of paragraphs 1-17, wherein the inhibitor comprises a modification selected from the group consisting of nucleobase modifications, sugar modifications, inter-sugar linkage modifications, backbone modifications, and any combinations thereof.
19. The method of any of paragraphs 1-18, wherein the inhibitor comprises a ligand.
20. The method of any of paragraphs 1-19, wherein the inhibitor is from 6 to 50 nucleotides in length.
21. The method of any of paragraphs 1-20, wherein the inhibitor is a single-stranded nucleic acid.
22. The method of any of paragraphs 1-21, wherein the inhibitor is encoded by an expression vector.
23. The method of any of paragraphs 1-22, wherein the inhibitor is formulated in a lipid delivery vehicle.
24. The method of any of paragraphs 1-23, wherein the inhibitor is formulated as a Staramine nanocomplex.
25. The method of any of paragraphs 1-24, wherein the inhibitor decreases the amount or expression of the at least one member of miR-130/301 relative to a control or reference level.
26. The method of any of paragraphs 1-25, wherein the inhibitor increases the amount of peroxisome proliferator-activated receptor gamma (PPARγ), a nucleic acid encoding PPARγ, apelin, a nucleic acid encoding apelin, nitric oxide synthase(NOS3), a nucleic acid encoding NOS3, TIMP2, a nucleic acid encoding TIMP2, miR-424, miR-503, miR-204, COL1A1, a nucleic acid encoding COL1A1, COL3A1, a nucleic acid encoding COL3A1, CTGF, a nucleic acid encoding CTGF, LOX or a nucleic acid encoding LOX relative to a control or reference level.
27. The method of any of paragraphs 1-26, wherein the nucleic acid encoding PPARγ, apelin, NOS3, TMP2, CTGF or LOX is mRNA.
28. The method of any of paragraphs 1-27, wherein the inhibitor increases the amount of apelin, the nucleic acid encoding apelin, or miR-424/503 in a pulmonary arterial endothelial cell (PAEC).
29. The method of any of paragraphs 1-28, wherein the inhibitor increases the amount of miR-204 in a pulmonary arterial smooth muscle cell (PASMC).
30. The method of any of paragraphs 1-29, wherein inhibitor decreases the amount of FGF2, a nucleic acid encoding FGF2, STAT3, a nucleic acid encoding STAT3, phosphorylated STATA3, vascular growth factor-A (VEGFA), a nucleic acid encoding VEGFA, endothelin-1 (EDN1), a nucleic acid encoding EDN1, MMP2 or a nucleic acid encoding MMP2 relative to a control or reference level.
31. The method of any of paragraphs 1-30, wherein the nucleic acid encoding FGF2, STAT3, VEGFA EDN1 or MMP2 is mRNA.
32. The method of any of paragraphs 1-31, wherein the inhibitor decreases the amount of FGF2 or the nucleic acid encoding FGF2 in PAEC.
33. The method of any of paragraphs 1-32, wherein the inhibitor decreases the amount of STAT3 or the nucleic acid encoding STAT3 in a PASMC.
34. The method of any of paragraphs 1-33, wherein the inhibitor decreases cellular contraction.
35. The method of any of paragraphs 1-34, wherein the inhibitor decreases cellular proliferation.
36. The method of any of paragraphs 1-35, further comprising selecting a subject for treatment before onset of said administering, comprising assaying a biological sample from the subject for miR-130/301 and selecting the subject who has elevated level of at least one (e.g., one, two, three, four, five, six, seven, or eight) member of miR-130/301 family.
37. The method of paragraph 36, wherein the miR-130/301 family member is selected from the group consisting of miR-130a, miR-130b, miR-301a, miR-301b, and any combinations thereof.
38. The method of any of paragraphs 1-37, further comprising co-administering a therapeutic agent, wherein the therapeutic agent is for treatment of pulmonary hypertension, PAH or fibrotic disease.
39. The method of any of paragraphs 1-38, wherein the therapeutic agent is for treatment of pulmonary hypertension and is selected from the group consisting of statins, diuretics, adrenergic receptor antagonists, PDE5 inhibitors, calcium channel blockers, renin inhibitors, ACE inhibitors, angiotensin II receptor antagonists, aldosterone antagonists, vasodilators; alpha-2-agonists, and any combination thereof.
40. The method of any of paragraphs 1-39, wherein the therapeutic agent is selected from the group consisting of bumetanide; ethacrynic acid; furosemide; torsemide; epitizide; hydrochlorothiazide; chlorothiazide; bendroflumethiazide; indapamide; chlorthalidone; metolazone; amiloride; triamterene; spironolactone; atenolol; metoprolol; nadolol; oxprenolol; pindolol; propranolol; timolol; doxazosin; phentolamine; indoramin; phenoxybenzamine; prazosin; terazosin; tolazoline; bucindolol; carvedilol; labetalol; amlodipine; felodipine; isradipine; lercanidipine; nicardipine; nifedipine; nimodipine; nitrendipine; diltiazem; verapamil; Aliskiren; captopril; enalapril; fosinopril; lisinopril; perindopril; quinapril; ramipril; trandolapril; benazepril; candesartan; eprosartan; irbesartan; losartan; olmesartan; telmisartan; valsartan; eplerenone; spironolactone; sodium nitroprusside; hydralazine; hydralazine derivatives; Clonidine; Guanabenz; Methyldopa; Moxonidine; Guanethidine; Reserpine; atorvastatin; fluvastatin; lovastatin; pitavastatin; pravastatin; rosuvastatin; simvastatin; and any combinations thereof.
41. A synthetic oligonucleotide comprising a nucleotide sequence that is substantially complementary to a at least a portion of nucleic acid sequence 5′-AGUGCAA-3′ (SEQ ID NO: 1)
42. The oligonucleotide of paragraph 41, wherein the oligonucleotide comprises a nucleotide sequence that is substantially complementary to at least a portion of nucleic acid sequence selected from the group consisting of cagugcaauguuaaaagggcau (hsa-miR-130a-3p), (cagugcaaugaugaaagggcau (hsa-miR-130b-3p), cagugcaauaguauugucaaagc (has-miR-301a-3p), cagugcaaugauauugucaaagc (hsa-miR-301b-3p), uagugcaauauugcuuauagggu (hsa-miR-454-3p), and any combinations thereof.
43. The oligonucleotide of any of paragraphs 41-42, wherein the oligonucleotide comprises the nucleotide sequence 5′-TTGCACT-3′(SEQ ID NO: 2) or 5′-ATTGCACT-3′ (SEQ ID NO: 3).
44. The oligonucleotide of any of paragraphs 41-43, wherein the oligonucleotide comprises a modification selected from the group consisting of nucleobase modifications, sugar modifications, inter-sugar linkage modifications, backbone modifications, and any combinations thereof.
45. The oligonucleotide of any of paragraphs 41-44, wherein the oligonucleotide comprises a ligand.
46. The oligonucleotide of any of paragraphs 41-45, wherein the oligonucleotide is from 6 to 50 nucleotides in length.
47. The oligonucleotide of any of paragraphs 31-46, wherein the oligonucleotide is an anti-miR, antagomir, antisense oligonucleotide, ribozyme, siRNA, or shRNA.
48. A kit comprising a synthetic oligonucleotide of any of paragraphs 41-47.
49. A pharmaceutical composition comprising a synthetic oligonucleotide of any of paragraphs 41-47.
50. An expression vector encoding an oligonucleotide of any of paragraphs 31-47.
51. A pharmaceutical composition comprising an expression vector of paragraph 50.
52. An assay comprising: measuring or quantifying the amounts of microRNA-130a, microRNA-130b, microRNA-301a and microRNA-301b in a biological sample obtained from a subject; and comparing the measured or quantified amounts with a reference value, and if the amounts are increased relative to the reference value, identifying the subject as having an increased probability of having pulmonary hypertension or a fibrotic or fibroproliferative disease.
53. An assay to determine if a subject is at risk of pulmonary hypertension, or PAH, or a fibrotic or fibroproliferative disease, or increaded extracellular matrix deposition, the assay comprising contacting a biological sample obtained from the subject with probes to detect the levels of microRNA-130a, microRNA-130b, microRNA-301a and microRNA-301b, wherein the level of microRNA-130a, microRNA-130b, microRNA-301a and microRNA-301b above a predefined reference level identifies the subject predicted to be at risk of pulmonary hypertension or PAH, a fibrotic or fibroproliferative disease, or increased extracellular matrix deposition.
54. The assay of paragraph 53, wherein the fibrotic or fibroproliferative disease is cancer/metastasis.

### Some Selected Definition

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The terms “microRNA” or “miRNA” or “mir” or “miR” are used interchangeably herein, are endogenous RNAs, some of which are known to regulate the expression of protein-coding genes at the posttranscriptional level. As used herein, the term “microRNA” refers to any type of micro-interfering RNA, including but not limited to, endogenous microRNA and artificial microRNA. Typically, endogenous microRNA are small RNAs encoded in the genome which are capable of modulating the productive utilization of mRNA. A mature miRNA is a single-stranded RNA molecule of about 21-23 nucleotides in length which is complementary to a target sequence, and hybridizes to the target RNA sequence to inhibit expression of a gene which encodes a miRNA target sequence. miRNAs themselves are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. MicroRNA sequences have been described in publications such as, Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into the precursor molecule.

A mature miRNA is produced as a result of a series of miRNA maturation steps; first a gene encoding the miRNA is transcribed. The gene encoding the miRNA is typically much longer than the processed mature miRNA molecule; miRNAs are first transcribed as primary transcripts or “pri-miRNA” with a cap and poly-A tail, which is subsequently processed to short, about 70-nucleotide “stem-loop structures” known as “pre-miRNA” in the cell nucleus. This processing is performed in animals by a protein complex known as the Microprocessor complex, consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). This complex is responsible for the gene silencing observed due to miRNA expression and RNA interference. The pathway is different for miRNAs derived from intronic stem-loops; these are processed by Drosha but not by Dicer. In some instances, a given region of DNA and its complementary strand can both function as templates to give rise to at least two miRNAs. Mature miRNAs can direct the cleavage of mRNA or they can interfere with translation of the mRNA, either of which results in reduced protein accumulation, rendering miRNAs capable of modulating gene expression and related cellular activities.

The term “pri-miRNA” refers to a precursor to a mature miRNA molecule which comprises; (i) a microRNA sequence and (ii) stem-loop component which are both flanked (i.e. surrounded on each side) by “microRNA flanking sequences”, where each flanking sequence typically ends in either a cap or poly-A tail. A pri-microRNA, (also referred to as large RNA precursors), are composed of any type of nucleic acid based molecule capable of accommodating the microRNA flanking sequences and the microRNA sequence. Examples of pri-miRNAs and the individual components of such precursors (flanking sequences and microRNA sequence) are provided herein. The nucleotide sequence of the pri-miRNA precursor and its stem-loop components can vary widely. In one aspect a pre-miRNA molecule can be an isolated nucleic acid; including microRNA flanking sequences and comprising a stem-loop structure and a microRNA sequence incorporated therein. A pri-miRNA molecule can be processed in vivo or in vitro to an intermediate species caller “pre-miRNA”, which is further processed to produce a mature miRNA.

The term “pre-miRNA” refers to the intermediate miRNA species in the processing of a pri-miRNA to mature miRNA, where pri-miRNA is processed to pre-miRNA in the nucleus, whereupon pre-miRNA translocates to the cytoplasm where it undergoes additional processing in the cytoplasm to form mature miRNA. Pre-miRNAs are generally about 70 nucleotides long, but can be less than 70 nucleotides or more than 70 nucleotides.

The term “microRNA flanking sequence” as used herein refers to nucleotide sequences including microRNA processing elements. MicroRNA processing elements are the minimal nucleic acid sequences which contribute to the production of mature miRNA from precursor microRNA. Often these elements are located within a 40 nucleotide sequence that flanks a microRNA stem-loop structure. In some instances the microRNA processing elements are found within a stretch of nucleotide sequences of between 5 and 4,000 nucleotides in length that flank a microRNA stem-loop structure. Thus, in some embodiments the flanking sequences are 5-4,000 nucleotides in length. As a result, the length of the precursor molecule can be, in some instances at least about 150 nucleotides or 270 nucleotides in length. The total length of the precursor molecule, however, can be greater or less than these values. In other embodiments the minimal length of the microRNA flanking sequence is 10, 20, 30, 40, 50, 60, 70, 80, 90, 100, 150, 200 and any integer there between. In other embodiments the maximal length of the microRNA flanking sequence is 2, 000, 2,100, 2, 200, 2,300, 2,400, 2,500, 2,600, 2,700, 2,800, 2,900, 3, 000, 3,100, 3,200, 3,300, 3,400, 3,500, 3,600, 3,700, 3,800, 3,900 4,000 and any integer there between.

MicroRNA flanking sequences can be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. microRNA flanking sequences within the pri-miRNA molecule can flank one or both sides of the stem-loop structure encompassing the microRNA sequence. Thus, one end (i.e., 5′) of the stem-loop structure can be adjacent to a single flanking sequence and the other end (i.e., 3′) of the stem-loop structure cannot be adjacent to a flanking sequence. Preferred structures have flanking sequences on both ends of the stem-loop structure. The flanking sequences can be directly adjacent to one or both ends of the stem-loop structure or can be connected to the stem-loop structure through a linker, additional nucleotides or other molecules.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem can include one or more base mismatches. Alternatively, the base-pairing can be exact, i.e. not include any mismatches. In some instances the precursor microRNA molecule can include more than one stem-loop structure. The multiple stem-loop structures can be linked to one another through a linker, such as, for example, a nucleic acid linker or by a microRNA flanking sequence or other molecule or some combination thereof.

Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways. As used herein, the term “miRNA mimetic” refers to an artificial miRNA or RNAi (RNA interference molecule) which is flanked by the stem-loop like structures of a pri-miRNA.

The term “artificial microRNA” includes any type of RNA sequence, other than endogenous microRNA, which is capable of modulating the productive utilization of mRNA. For instance, the term artificial microRNA also encompasses a nucleic acid sequence which would be previously identified as siRNA, where the siRNA is incorporated into a vector and surrounded by miRNA flanking sequences as described herein.

As used herein, “double stranded RNA” or “dsRNA” refers to RNA molecules that are comprised of two substantially complementary strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

As used herein, “gene silencing” or “gene silenced” by a miRNA and/or RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein a “siRNA” refers to a nucleic acid that forms a double stranded RNA, which double stranded RNA has the ability to reduce or inhibit expression of a particular gene or target gene when the siRNA is expressed in the same cell as the gene or target gene. The double stranded RNA siRNA can be formed by the complementary strands. The complementary portions of the siRNA that hybridize to form the double stranded molecule typically have substantial or complete identity. In one embodiment, a siRNA refers to a nucleic acid that has substantial or complete identity to sequence of a target gene and forms a double stranded RNA. The sequence of the siRNA can correspond to the full length target gene, or to a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The term “biological sample” as used herein means a sample of biological tissue or fluid that comprises nucleic acids. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

The term “tissue” refers to a group or layer of similarly specialized cells which together perform certain special functions. The term “tissue-specific” refers to a source of cells from a specific tissue. The term “tissue” is intended to include, blood, blood preparations such as plasma and serum, bones, joints, muscles, smooth muscles, and organs.

The terms “disease” or “disorder” are used interchangeably herein, and refer to any alteration in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition or affliction.

The terms “subject” and “individual” are used interchangeably herein, and mean a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of PH, PAH, or fibrotic or fibroproliferative disease.

In addition, the methods described herein can be used to treat domesticated animals and/or pets. A subject can be male or female. A subject can be one who has been previously diagnosed with or identified as suffering from or having of PH, PAH, or fibrotic or fibroproliferative disease, but need not have already undergone treatment.

In some embodiments, subject amenable to treatment with the anti-miR-130/301 agents have a blood pressure of above 120/80 mm Hg, above 115/75 mm Hg. In some embodiments, subject amenable to treatment with the anti-miR-130/301 agents have pre-hypertension, where their pre-hypertension is a systolic pressure ranges from 120 to 139 mm Hg or a diastolic pressure ranges from 80 to 89 mm Hg. In some embodiments, subject amenable to treatment with the anti-miR-130/301 agents have stage 1 or Stage 2 hypertension, where subjects with stage 1 hypertension have a systolic pressure ranging from about 90-100 mm Hg to about 159 mm Hg or a diastolic pressure ranging from 90 to 99 mm Hg, and subjects with stage 2 hypertension have a systolic pressure of 160 mm Hg or higher or a diastolic pressure of 100 mm Hg or higher.

The term “effective amount” as used herein refers to the amount of therapeutic agent of pharmaceutical composition to alleviate at least one of the symptoms of the disease or disorder.

The term “gene” as used herein refers to a genomic gene comprising transcriptional and/or translational regulatory sequences and/or a coding region and/or non-translated sequences (e.g., introns, 5′- and 3′-untranslated sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto. The term “encoding nucleic acid” is intended to include the term “gene.”

The term “probe” as used herein refers to an oligonucleotide capable of binding to a target nucleic acid of complementary sequence through one or more types of chemical bonds, usually through complementary base pairing, usually through hydrogen bond formation. Probes can bind target sequences lacking complete complementarily with the probe sequence depending upon the stringency of the hybridization conditions. There can be any number of base pair mismatches which will interfere with hybridization between the target sequence and single stranded target nucleic acids, but a probe will bind a selected target specifically, i.e. to the substantial exclusion of non-target nucleic acids under at least one set of conditions. A probe can be single stranded or partially single and partially double stranded. A probe will generally be detectably labeled or carry a moiety that permits signal detection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see below) are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. The term “substantially homologous” refers to sequences that are at least 90%, at least 95% identical, at least 97% identical or at least 99% identical. Homologous sequences can be the same functional gene in different species.

Determination of homologs of the genes or peptides can be easily ascertained by the skilled artisan. The terms “homology”, “identity” and “similarity” refer to the degree of sequence similarity between two peptides or between two optimally aligned nucleic acid molecules. Homology and identity can each be determined by comparing a position in each sequence which can be aligned for purposes of comparison. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with a sequence of the present application.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or l) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the; percentage of sequence identity is calculated by comparing the reference sequence to the sequence which can include deletions or additions which total 20 percent or less of the reference sequence over the comparison window. The reference sequence can be a subset of a larger sequence. The term “similarity”, when used to describe a polypeptide, is determined by comparing the amino acid sequence and the conserved amino acid substitutes of one polypeptide to the sequence of a second polypeptide.

The term “vectors” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression or the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome.

As used herein, the terms “treat” or “treatment” or “treating” as used herein refers to therapeutic treatment, wherein the object is to prevent or slow the development of the disease, such as slow down the development of or reducing at least one adverse effect or symptom of a the disease or disorder. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with PH, PAH, or fibrotic or fibroproliferative disease, as well as those likely to develop such a condition due to genetic susceptibility or other factors such as weight, diet and health. In some embodiments, the term to treat also encompasses preventative measures and/or prophylactic treatment, which includes administering a pharmaceutical composition as disclosed herein to prevent the onset of a disease or disorder.

In some embodiment, the term “treating” when used in reference to a treatment of PH, PAH or fibrotic or fibroproliferative disease or disorder is used to refer to the reduction of a symptom and/or a biochemical marker of PH, PAH or fibrotic or fibroproliferative disease or disorder, for example a reduction in at least one biochemical marker of PH, PAH or fibrotic or fibroproliferative disease or disorder by at least about 10% would be considered an effective treatment. Subjects amenable to treatment according to the methods as disclosed herein can be identified by any method to diagnose PH, PAH, fibrotic or fibroproliferative disease.

The term “effective amount” as used herein refers to the amount of therapeutic agent of pharmaceutical composition to alleviate at least one or more symptom of the disease or disorder, and relates to a sufficient amount of pharmacological composition to provide the desired effect.

The phrase “therapeutically effective amount” as used herein, means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically significant reduction in a symptom or clinical marker associated with PH, PAH or fibrotic or fibroproliferative disease or disorder.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

The term “hypertension” is also referred to as “HTN” or “high blood pressure” or “arterial hypertension” refers to a medical condition in which the blood pressure in the arteries is elevated. This requires the heart to work harder than normal to circulate blood through the blood vessels. Blood pressure is summarised by two measurements, systolic and diastolic, which depend on whether the heart muscle is contracting (systole) or relaxed between beats (diastole) and equate to a maximum and minimum pressure, respectively. Normal blood pressure at rest is within the range of 100-140 mmHg systolic (top reading) and 60-90 mmHg diastolic (bottom reading). High blood pressure is said to be present if it is persistently at or above 140/90 mmHg. Without wishing to be bound by theory, hypertension is classified as either primary (essential) hypertension or secondary hypertension; about 90-95% of cases are categorized as “primary hypertension” which means high blood pressure with no obvious underlying medical cause. The remaining 5-10% of cases (secondary hypertension) are caused by other conditions that affect the kidneys, arteries, heart or endocrine system. Hypertension is a major risk factor for stroke, myocardial infarction (heart attacks), heart failure or chronic heart failure (CHF), aneurysms of the arteries (e.g. aortic aneurysm), peripheral arterial disease and is a cause of chronic kidney disease. Even moderate elevation of arterial blood pressure is associated with a shortened life expectancy. Dietary and lifestyle changes can improve blood pressure control and decrease the risk of associated health complications, although drug treatment is often necessary in people for whom lifestyle changes prove ineffective or insufficient.

Methods of diagnosing essential or secondary hypertension are well known to those of skill in the art (see, e.g., Isselbacher et al. (1994) Harrison's Principles of Internal Medicine, 13.sup.th Ed., McGraw-Hill, Inc., New York). Physical examination and laboratory tests are directed at (1) uncovering correctable secondary forms of hypertension; (2) establishing a pretreament baseline, (3) assessing factors which may influence the type or therapy or which may be adversely modified by therapy, (4) determining if target organ damage is present and (5) determining whether other risk factors for the development or arteriosclerotic cardiovascular diseases are present.

As used herein, the term “pulmonary hypertension” or “PH” includes groups 1 to 4 according the updated clinical classification (Group 1: Pulmonary arterial hypertension (PAH); Group 2: Pulmonary hypertension with left heart disease; Group 3: Pulmonary hypertension associated with lung diseases and/or hypoxemia; Group 4: Pulmonary hypertension owing to chronic thrombotic and/or embolic disease) (Updated clinical classification of pulmonary hypertension. Simonneau G, Robbins I M, Beghetti M, Channick R N, Delcroix M, Denton C P, Elliott C G, Gaine S P, Gladwin M T, Jing Z C, Krowka M J, Langleben D, Nakanishi N, Souza R. J Am Coll Cardiol. 2009; 54 (Suppl): S43-54).

As used herein, the term “pulmonary arterial hypertension” or “PAH” is intended to include idiopathic PAH, familial PAH, pulmonary veno-occlusive disease (PVOD), pulmonary capillary hemangiomatosis (PCH), persistent pulmonary hypertension of the newborn, or PAH associated with another disease or condition, such as, but not limited to, collagen vascular disease, congenital systemic-to-pulmonary shunts (including Eisenmenger's syndrome), portal hypertension, HIV infection, drugs and toxins, thyroid disorders, glycogen storage disease, Gaucher disease, hereditary hemorrhagic telangiectasia, hemoglobinopathies, myeloproliferative disorders, or splenectomy.

A “fibrotic” disease or a “fibroproliferative” disease refers to a disease characterized by scar formation and the over production of collagen or other extracellular matrix (ECM) by connective tissue, i.e., a disease or disorder characterized by an increase in fibrous connective tissue in an organ or tissue, such as increases in collagen or other extracellular matrix components relative to non afflicted controls. Fibrotic disease occurs as a result of tissue damage. It can occur in virtually every organ of the body. Examples of fibrotic or fibroproliferative diseases include, but are not limited to, idiopathic pulmonary fibrosis, fibrotic interstitial lung disease, interstitial pneumonia, fibrotic variant of non-specific interstitial pneumonia, cystic fibrosis, lung fibrosis, silicosis, asbestosis, asthma, chronic obstructive pulmonary lung disease (COPD), pulmonary arterial hypertension, liver fibrosis, liver cirrhosis, renal fibrosis, glomerulosclerosis, x kidney fibrosis, diabetic nephropathy, heart disease, fibrotic valvular heart disease, systemic fibrosis, rheumatoid arthritis, excessive scarring resulting from surgery, chemotherapeutic drug-induced fibrosis, radiation-induced fibrosis, macular degeneration, retinal and vitreal retinopathy, atherosclerosis, and restenosis. Fibrotic disease or disorder, fibroproliferative disease or disorder and fibrosis are used interchangeably herein.

“Fibrotic disease” or conditions include, but are not limited to hepatic cirrhosis, congestive heart failure, fibrotic lung disease, photo-aging, cystic fibrosis of the pancreas and lungs, injection fibrosis, which can occur as a complication of intramuscular injections, endomyocardial fibrosis, idiopathic pulmonary fibrosis of the lung, mediastinal fibrosis, myelofibrosis, retroperitoneal fibrosis, progressive massive fibrosis (a complication of coal workers' pneumoconiosis, nephrogenic systemic fibrosis, scleroderma, kidney fibrosis, fibrosis related to organ transplants, scars, burns and the like.

Liver (hepatic) fibrosis, for example, occurs as a part of the wound-healing response to chronic liver injury. Fibrosis occurs as a complication of haemochromatosis, Wilson's disease, alcoholism, schistosomiasis, viral hepatitis, bile duct obstruction, exposure to toxins, and matabolic disorders. This formation of scar tissue is believed to represent an attempt by the body to encapsulate the injured tissue. Liver fibrosis is characterized by the accumulation of extracellular matrix that can be distinguished qualitatively from that in normal liver. Left unchecked, hepatic fibrosis progresses to cirrhosis (defined by the presence of encapsulated nodules), liver failure, and death.

In some embodiments, the fibrotic disease can be selected from the group consisting of: atherosclerosis, asthma, cardiac fibrosis, organ transplant fibrosis, colloid and hypertrophic scar, muscle fibrosis, pancreatic fibrosis, bone-marrow fibrosis, liver fibrosis, cirrhosis of liver and gallbladder, scleroderma, pulmonary fibrosis, diffuse parenchymal lung disease, idiopathic interstitial fibrosis, interstitial pneumonitis, desquamative interstitial pneumonia, respiratory bronchiolitis, interstitial lung disease, acute interstitial pneumonitis, nonspecific interstitial pneumonia, cryptogenic organizing pneumonia, lymphocytic interstitial pneumonia, renal fibrosis, or chronic kidney disease.

In some embodiments the fibroproliferative disease or disorder is cancer or metastasis. As used herein, the term “cancer” refers to an uncontrolled growth of cells that may interfere with the normal functioning of the bodily organs and systems. Cancers that migrate from their original location and seed vital organs can eventually lead to the death of the subject through the functional deterioration of the affected organs. Metastasis is a cancer cell or group of cancer cells, distinct from the primary tumor location resulting from the dissemination of cancer cells from the primary tumor to other parts of the body. At the time of diagnosis of the primary tumor mass, the subject may be monitored for the presence of in transit metastases, e.g., cancer cells in the process of dissemination. As used herein, the term cancer, includes, but is not limited to the following types of cancer, breast cancer, biliary tract cancer, bladder cancer, brain cancer including Glioblastomas and medulloblastomas; cervical cancer; choriocarcinoma; colon cancer; endometrial cancer; esophageal cancer, gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma, Wilms tumor. Examples of cancer include but are not limited to, carcinoma, including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, Glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Other cancers will be known to the artisan.

As used herein, the term “cancer” includes, but is not limited to, solid tumors and blood born tumors. The term cancer refers to disease of skin, tissues, organs, bone, cartilage, blood and vessels. The term “cancer” further encompasses primary and metastatic cancers. Examples of cancers that can be treated with the compounds of the invention include, but are not limited to, carcinoma, including that of the bladder, breast, colon, kidney, lung, ovary, pancreas, stomach, cervix, thyroid, and skin, including squamous cell carcinoma; hematopoietic tumors of lymphoid lineage, including, but not limited to, leukemia, acute lymphocytic leukemia, acute lymphoblastic leukemia, B-cell lymphoma, T-cell lymphoma, Hodgkins lymphoma, non-Hodgkins lymphoma, hairy cell lymphoma, and Burketts lymphoma; hematopoietic tumors of myeloid lineage including, but not limited to, acute and chronic myelogenous leukemias and promyelocytic leukemia; tumors of mesenchymal origin including, but not limited to, fibrosarcoma, rhabdomyosarcoma, and osteosarcoma; other tumors including melanoma, seminoma, tetratocarcinoma, neuroblastoma, and glioma; tumors of the central and peripheral nervous system including, but not limited to, astrocytoma, neuroblastoma, glioma, and schwannomas; and other tumors including, but not limited to, xenoderma, pigmentosum, keratoactanthoma, thyroid follicular cancer, and teratocarcinoma. The methods disclosed herein are useful for treating patients who have been previously treated for cancer, as well as those who have not previously been treated for cancer. Indeed, the methods and compositions of this invention can be used in first-line and second-line cancer treatments.

As used herein, the term “precancerous condition” has its ordinary meaning, i.e., an unregulated growth without metastasis, and includes various forms of hyperplasia and benign hypertrophy. Accordingly, a “precancerous condition” is a disease, syndrome, or finding that, if left untreated, can lead to cancer. It is a generalized state associated with a significantly increased risk of cancer. Premalignant lesion is a morphologically altered tissue in which cancer is more likely to occur than its apparently normal counterpart. Examples of pre-malignant conditions include, but are not limited to, oral leukoplakia, actinic keratosis (solar keratosis), Barrett's esophagus, atrophic gastritis, benign hyperplasia of the prostate, precancerous polyps of the colon or rectum, gastric epithelial dysplasia, adenomatous dysplasia, hereditary nonpolyposis colon cancer syndrome (HNPCC), Barrett's esophagus, bladder dysplasia, precancerous cervical conditions, and cervical dysplasia.

As used herein, the terms “administer”, “administering,” and “introducing” are used interchangeably herein and refer to the the placement of a composition into a subject by a method or route which results in at least partial localization of the composition at a desired site such that desired effect is produced. The compound or composition can be administered by any appropriate route known in the art which results in an effective treatment in the subject, including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration therapeutic compositions other than directly into a tumor such that it enters the animal's system and, thus, is subject to metabolism and other like processes.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

The term “agent” refers to any entity which is normally not present or not present at the levels being administered to a cell, tissue or subject. Agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. Agents can be known to have a desired activity and/or property, or can be selected from a library of diverse compounds.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The technology described herein is further illustrated by the following examples which in no way should be construed as being further limiting, The contents of all cited references, including literature references, issued patents, published patent applications, and co-pending patent applications, cited throughout this application are hereby expressly incorporated by reference.

The technology described herein has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: Systems-Level Regulation of MicroRNA Networks by miR-130/301Promotes Pulmonary Hypertension

Pulmonary hypertension (PH) is a vascular disease involving disparate molecular pathways spanning multiple cell types. MicroRNAs (miRNAs) may coordinately regulate these processes, but their integrative functions have been challenging to define with conventional approaches. Guided by analysis of the molecular network architecture specific to PH, the miR-130/301 family was predicted as a master regulator of cellular proliferation in PH via control of subordinate miRNA pathways with previously unidentified connections to one another. In validation of this model, up-regulation of miR-130/301 expression was confirmed in diseased pulmonary vessels and plasma from mammalian and human PH subjects. The miR-130/301 family targeted PPARγ with distinct cell type-specific consequences. To promote proliferation, these miRNAs modulated apelin-miR-424/503-FGF2 signaling in pulmonary arterial endothelial cells and separately modulated STAT3-miR-204 signaling in pulmonary arterial smooth muscle cells. Finally, we found the miR-130/301 family necessary and sufficient to control this entire pathogenic hierarchy and promote PH in mice. Such results clarify our deficient understanding of the systems-level regulation of miRNA-disease gene networks in PH with broad implications on miRNA-based therapeutics in this disease. Furthermore, these findings provide critical validation for the evolving application of network theory to the discovery of the miRNA-based origins of PH and other diseases.

We identified the miR-130/301 family, the top-ranked miRNA family derived from our network-based predictions, as such a molecular integrator in PH—repressing its direct target PPARγ in order to control the STAT3-miR-204 pathway in PASMCs, the apelin-miR-424/503-FGF2 pathway in PAECs, and ultimately, cellular proliferation and downstream hemodynamic manifestations of PH in vivo. These findings have important implications in advancing our understanding of the network hierarchy of PH, guiding miRNA-based therapeutic strategies in this disease, and offering critical support for the application of network theory to define such higher-order regulation in complex diseases such as PH.

### Results

In Silico Prediction of the miR-130/301 Family as a Master Regulator of PH:

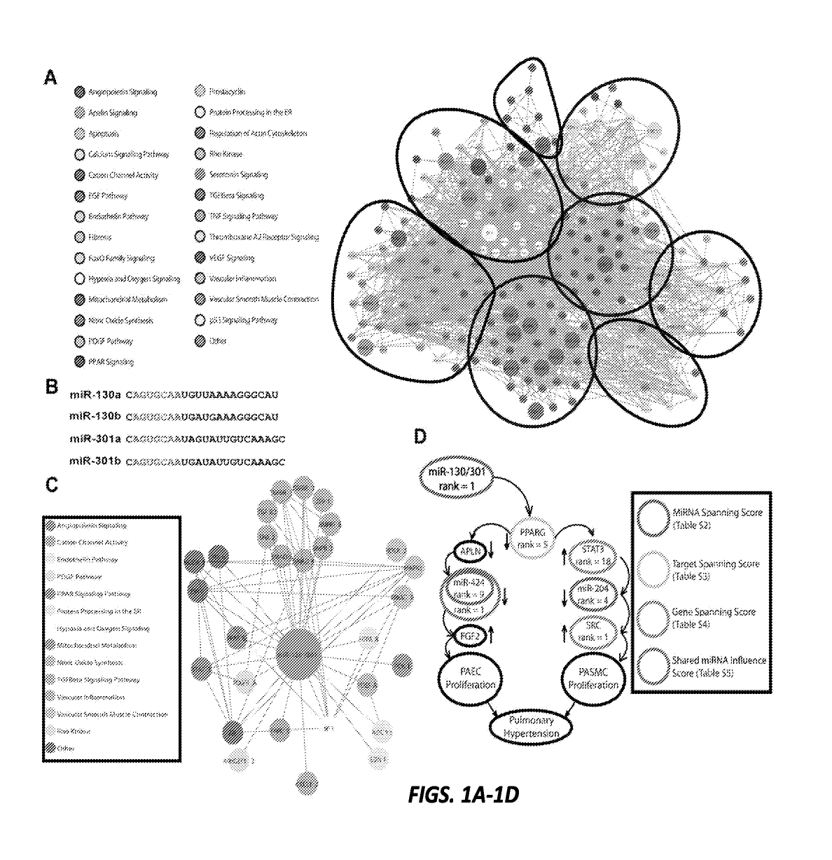

To leverage the advancing principles of network theory for determining systems-level molecular regulators of PH pathogenesis, a network of genes and interactions were constructed in silico based on curated seed genes with known importance in PH [as previously described (9)] and their first degree interactors (see Methods for details). Interactions were defined from a master list of functional molecular associations (i.e., the “consolidated interactome”) catalogued from a set of databases containing all known human gene and molecular interactions (10). The complete network were refer to as the “expanded PH network,” containing 249 nodes and 2,274 edges with the largest connected component encompassing all 249 nodes (FIG. 1A, Table 1).

TABLE 1
Genes in the expanded PH network. Genes are annotated by
architectural cluster and PH-relevant functional pathway.

| Cluster | Gene | Functional Pathway |
|---|---|---|
| Cluster 1 | AKT1 | TNF Signaling Pathway |
| | ANGPT4 | Angiopoietin Signaling |
| | BCL2 | Apoptosis |
| | BIRC4 | Apoptosis |
| | KIAA1967 | Apoptosis |
| | CAMK2G | Calcium Signaling Pathway |
| | KCNA5 | Cation Channel Activity |
| | KCND2 | Cation Channel Activity |
| | KCNIP2 | Cation Channel Activity |
| | TRPC1 | Cation Channel Activity |
| | TRPC3 | Cation Channel Activity |
| | TRPC6 | Cation Channel Activity |
| | SOD2 | Mitochondrial Metabolism |
| | GCH1 | Nitric Oxide Synthesis |
| | GUCY1A2 | Nitric Oxide Synthesis |
| | GUCY1A3 | Nitric Oxide Synthesis |
| | GUCY1B2 | Nitric Oxide Synthesis |
| | GUCY1B3 | Nitric Oxide Synthesis |
| | NOS1 | Nitric Oxide Synthesis |
| | NOS2A | Nitric Oxide Synthesis |
| | NOS3 | Nitric Oxide Synthesis |
| | PDE1A | Nitric Oxide Synthesis |
| | PDE1C | Nitric Oxide Synthesis |
| | PDE2A | Nitric Oxide Synthesis |
| | PDE3A | Nitric Oxide Synthesis |
| | PDE6D | Nitric Oxide Synthesis |
| | PDE7A | Niitric Oxide Signaling |
| | PDE9A | Nitric Oxide Synthesis |
| | PTGIR | Prostacyclin |
| | HSP90AA1 | Protein Processing in the ER |
| | CHUK | TNF Signaling Pathway |
| | GTF2I | TNF Signaling Pathway |
| | IKBKE | TNF Signaling Pathway |
| | MAP1LC3B | TNF Signaling Pathway |
| | MAPK3 | TNF Signaling Pathway |
| | MAPK8 | TNF Signaling Pathway |
| | NFKB1 | TNF Signaling Pathway |
| | NFKB2 | TNF Signaling Pathway |
| | NFKBIE | TNF Signaling Pathway |
| | RELA | TNF Signaling Pathway |
| | TNFSF11 | TNF Signaling Pathway |
| | MAP3K8 | TGFbeta Signaling |
| | IL1B | Vascular Inflammation |
| | VCAM1 | Vascular Inflammation |
| | ROCK2 | Vascular Smooth Muscle Contraction |
| | ADCY5 | Vascular Smooth Muscle Contraction |
| | AGTR1 | Vascular Smooth Muscle Contraction |
| | CACNA1C | Vascular Smooth Muscle Contraction |
| | CALM1 | Vascular Smooth Muscle Contraction |
| | FGF2 | Vascular Smooth Muscle Contraction |
| | PRKACA | Vascular Smooth Muscle Contraction |
| | PRKACB | Vascular Smooth Muscle Contraction |
| | PRKACG | Vascular Smooth Muscle Contraction |
| | PRKCA | Vascular Smooth Muscle Contraction |
| | PRKCB1 | Vascular Smooth Muscle Contraction |
| | PRKCZ | Vascular Smooth Muscle Contraction |
| | PRKG1 | Vascular Smooth Muscle Contraction |
| | MAPK1 | Vascular Smooth Muscle Contraction |
| | DYNLL1 | Other |
| | DYNLT1 | Other |
| | GFAP | Other |
| | HIP2 | Other |
| | HMGCR | Other |
| | MBP | Other |
| | UBC | Other |
| Cluster 2 | MME | Fibrosis |
| | MMP1 | Fibrosis |
| | MMP14 | Fibrosis |
| | MMP2 | Fibrosis |
| | MMP3 | Fibrosis |
| | MMP9 | Fibrosis |
| | EPAS1 | Hypoxia and Oxygen Homeostasis |
| | FOS | Hypoxia and Oxygen Homeostasis |
| | HIF1A | Hypoxia and Oxygen Homeostasis |
| | HMOX1 | Hypoxia and Oxygen Homeostasis |
| | JUN | Hypoxia and Oxygen Homeostasis |
| | RBX1 | Hypoxia and Oxygen Homeostasis |
| | SERPINE1 | Hypoxia and Oxygen Homeostasis |
| | SMAD3 | Hypoxia and Oxygen Homeostasis |
| | SP1 | Hypoxia and Oxygen Homeostasis |
| | VHL | Hypoxia and Oxygen Homeostasis |
| | PDE5A | Nitric Oxide Synthesis |
| | CDKN2A | P53 Signaling Pathway |
| | EP300 | P53 Signaling Pathway |
| | PML | P53 Signaling Pathway |
| | THBS1 | P53 Signaling Pathway |
| | TP53 | P53 Signaling Pathway |
| | APOE | PPAR Signaling Pathway |
| | ILK | PPAR Signaling Pathway |
| | LRP1 | PPAR Signaling Pathway |
| | PPARG | PPAR Signaling Pathway |
| | RXRA | PPAR Signaling Pathway |
| | PTGIS | Prostacyclin |
| | PTGS2 | Prostacyclin |
| | AR | Rho Kinase |
| | CEBPB | Vascular Inflammation |
| | CYP1B1 | Vascular Inflammation |
| | NFATC2 | Vascular Inflammation |
| | NFATC3 | Vascular Inflammation |
| | VTNR | Vascular Inflammation |
| | EGR1 | Other |
| | ESR1 | Other |
| | NCOA3 | Other |
| | NOTCH1 | Other |
| | S100A11 | Other |
| | VIP | Other |
| Cluster 3 | APLN | Apelin Signaling |
| | MDFI | Apelin Signaling |
| | ANGPT1 | Angiopoietin Signaling |
| | ANGPT2 | Angiopoietin Signaling |
| | BMX | Angiopoietin Signaling |
| | FN1 | Angiopoietin Signaling |
| | STAT1 | Angiopoeitin Signaling |
| | STAT3 | Angiopoietin Signaling |
| | TEK | Angiopoietin Signaling |
| | EGF | EGF Pathway |
| | EGFR | EGF Pathway |
| | TNC | Fibrosis |
| | PDGFA | PDGF Pathway |
| | PDGFB | PDGF Pathway |
| | PDGFRA | PDGF Pathway |
| | PDGFRB | PDGF Pathway |
| | IL13 | Vascular Inflammation |
| | IL1A | Vascular Inflammation |
| | IL6 | Vascular Inflammation |
| | CAV1 | VEGF Signaling |
| | FYN | VEGF Signaling |
| | GRB2 | VEGF Signaling |
| | ITGB3 | VEGF Signaling |
| | KDR | VEGF Signaling |
| | PIK3R1 | VEGF Signaling |
| | PTK2B | VEGF Signaling |
| | PTPN1 | VEGF Signaling |
| | PTPN11 | VEGF Signaling |
| | SHC1 | VEGF Signaling |
| | SRC | VEGF Signaling |
| | VEGFA | VEGF Signaling |
| | PTEN | Other |
| | SH3KBP1 | Other |
| | SNX2 | Other |
| | GNB2L1 | Other |
| | HLA-DRA | Other |
| | HLA-DRB5 | Other |
| | HTATIP | Other |
| | IGF1R | Other |
| Cluster 4 | ACVRL1 | TGFbeta Signaling |
| | BMP2 | TGFbeta Signaling |
| | BMP4 | TGFbeta Signaling |
| | BMP6 | TGFbeta Signaling |
| | BMP7 | TGFbeta Signaling |
| | BMPR1A | TGFbeta Signaling |
| | BMPR1B | TGFbeta Signaling |
| | BMPR2 | TGFbeta Signaling |
| | DCN | TGFbeta Signaling |
| | EIF3I | TGFbeta Signaling |
| | ENG | TGFbeta Signaling |
| | FOXG1 | TGFbeta Signaling |
| | GDF5 | TGFbeta Signaling |
| | GREM1 | TGFbeta Signaling |
| | GREM2 | TGFbeta Signaling |
| | PARD3 | TGFbeta Signaling |
| | PPP1CA | TGFbeta Signaling |
| | SKIL | TGFbeta Signaling |
| | SMAD1 | TGFbeta Signaling |
| | SMAD2 | TGFbeta Signaling |
| | SMAD4 | TGFbeta Signaling |
| | SMAD5 | TGFbeta Signaling |
| | SMAD6 | TGFbeta Signaling |
| | SMAD7 | TGFbeta Signaling |
| | SMAD9 | TGFbeta Signaling |
| | TGFBI | TGFbeta Signaling |
| | TGFB1 | TGFbeta Signaling |
| | TGFB2 | TGFbeta Signaling |
| | TGFBR1 | TGFbeta Signaling |
| | TGFBR2 | TGFbeta Signaling |
| | TGFBR3 | TGFbeta Signaling |
| | ZEB1 | TGFbeta Signaling |
| | BTBD2 | TGFbeta Signaling |
| | HIPK2 | Other |
| | PABPC1 | Other |
| | SNRP70 | Other |
| Cluster 5 | ADCY1 | Endothelin Signaling |
| | EDN1 | Endothelin Signaling |
| | EDN2 | Endothelin Signaling |
| | EDN3 | Endothelin Signaling |
| | EDNRA | Endothelin Signaling |
| | PDE6G | Nitric Oxide Synthesis |
| | HTR1B | Serotonin Signaling |
| | HTR2B | Serotonin Signaling |
| | SLC6A4 | Serotonin Signaling |
| | ADRBK1 | Thromboxane A2 Receptor Signaling |
| | GNAI2 | Thromboxane A2 Receptor Signaling |
| | TBXA2R | Thromboxane A2 Receptor Signaling |
| | TGM2 | Thromboxane A2 Receptor Signaling |
| | CCL2 | Vascular Inflammation |
| | CCL3 | Vascular Inflammation |
| | CCL5 | Vascular Inflammation |
| | CCR1 | Vascular Inflammation |
| | CX3CL1 | Vascular Inflammation |
| | CX3CR1 | Vascular Inflammation |
| | CXCL12 | Vascular Inflammation |
| | HLA-B | Other |
| | PDCD6IP | Other |
| Cluster 6 | ACTA1 | Regulation of Actin Cytoskeleton |
| | ACTG1 | Regulation of Actin Cytoskeleton |
| | CFL1 | Regulation of Actin Cytoskeleton |
| | CFL2 | Regulation of Actin Cytoskeleton |
| | EZR | Regulation of Actin Cytoskeleton |
| | LIMK1 | Regulation of Actin Cytoskeleton |
| | LIMK2 | Regulation of Actin Cytoskeleton |
| | ARHGEF12 | Rho Kinase |
| | PAK1 | Rho Kinase |
| | RAC1 | Rho Kinase |
| | RHOA | Rho Kinase |
| | RHOB | Rho Kinase |
| | RHOG | Rho Kinase |
| | ROCK1 | Rho Kinase |
| | ICAM1 | Vascular Inflammation |
| | SELP | Vascular Inflammation |
| | ADD1 | Other |
| Cluster 7 | RTN4 | Other |
| | CSNK1A1 | FoxO Family Signaling |
| | CTNNB1 | FoxO Family Signaling |
| | HSPA5 | Protein Processing in the ER |
| | HSPA8 | Protein Processing in the ER |
| | STUB1 | Protein Processing in the ER |
| | ACTB | Regulation of Actin Cytoskeleton |
| | REM1 | Regulation of Actin Cytoskeleton |
| | CDKN1B | Vascular Inflammation |
| | GSK3B | VEGF Signaling |
| | RPS27A | Other |
| Cluster 8 | DLAT | Mitochondrial Metabolism |
| | FASN | Mitochondrial Metabolism |
| | MLYCD | Mitochondrial Metabolism |
| | PDHA1 | Mitochondrial Metabolism |
| | PDHA2 | Mitochondrial Metabolism |
| | PDHB | Mitochondrial Metabolism |
| | PDHX | Mitochondrial Metabolism |
| | PDK1 | Mitochondrial Metabolism |
| | PDK2 | Mitochondrial Metabolism |
| | PDK3 | Mitochondrial Metabolism |
| | PDK4 | Mitochondrial Metabolism |

To examine the possibility that a single miRNA may exert higher-order control over the expanded PH network as a whole, miRNAs were ranked based on the number and the spread of their predicted targets within this PH network. We chose the well-validated TargetScan 6.2 algorithm for miRNA target prediction, given its high degree of sensitivity and specificity relative to other algorithms of its kind (11). Because miRNAs often regulate multiple targets within the same functional pathway especially in PH (9), we reasoned that a miRNA was likely to have a robust association with the PH network if the size of its PH-relevant target pool was larger than would be expected by chance. Thus, we ranked conserved miRNAs according to a hypergeometric analysis of their number of PH-relevant targets, accounting for expanded PH network size and the total number of targets for each miRNA. We also reasoned that a miRNA with a holistic effect on PH progression would have a target pool that spanned a wide segment of the network. To capture that activity, inventors used a spectral partitioning-based algorithm (12) to divide the expanded PH network into clusters based on density of edges among genes in each cluster (encircled clusters as shown in FIG. 1A). MiRNAs were then ranked based on the number of distinct clusters in which they had at least one target. By combining the hypergeometric analysis and the cluster-based analysis into a single score, a “miRNA spanning score” was developed to reveal the capability of a given miRNA to modulate a diverse array of processes within the expanded PH network (Table 2). The miR-130/301 family was given the highest miRNA spanning score, both for having a large PH-relevant target pool (28 predicted targets depicted in FIG. 1C, listed in Table 3) and for having targets in every major network cluster (8 clusters). Of the eight miR-130/301 family members listed in TargetScan, we chose four members to study (miR-130a/b and miR-301a/b), based on their conserved expression in rodent and human pulmonary vascular cell types. Members of this miRNA family have been studied to a modest degree, mainly as isolated factors in cancer progression (13, 14) and non-PH related diseases (15-18). Notably, these miRNAs share the same seed sequence (nucleotides 2-8) and consequently putative targets (FIG. 1B), but they are encoded at separate chromosomal loci, indicating that distinct transcriptional events modulate their expression. Though it was not factored into our mathematical assessment of these miRNAs, it is worth noting that the 28 predicted targets of the miR-130/301 family represented a total of 13 functional pathways embedded throughout the network, based on annotation of gene function derived from the KEGG, Reactome, and NCBI BioSystems databases as well reports in the scientific literature (FIG. 1A, Table 1).

TABLE 2
Top thirty miRNA families ranked by their miRNA spanning score

| | Number | | | | |
|---|---|---|---|---|---|
| | of Clus- | Targets | Targets | | |
| | ters Tar- | in Net- | in Tar- | p- | Overall |
| miRNA | geted | work | getScan | value | Score |
| miR- | 8 | 28 | 638 | 0.00006 | 1.84437 |
| 130ac/301ab/301b/ | | | | | |
| 301b-3p/454/721/ | | | | | |
| 4295/3666 | | | | | |
| miR-27abc/27a-3p | 8 | 33 | 846 | 0.00007 | 1.83098 |
| miR-135ab/135a-5p | 8 | 24 | 515 | 0.00013 | 1.777211 |
| miR-204/204b/211 | 8 | 23 | 481 | 0.00014 | 1.770774 |
| miR-200bc/429/ | 6 | 32 | 771 | 0.00007 | 1.58098 |
| 548a | | | | | |
| miR-155 | 7 | 17 | 329 | 0.00032 | 1.57397 |
| miR-33ab/33-5p | 6 | 17 | 300 | 0.00013 | 1.527211 |
| miR-22/22-3p | 8 | 16 | 362 | 0.00257 | 1.518013 |
| miR- | 8 | 30 | 908 | 0.00467 | 1.466137 |
| 15abc/16/16abc/195/ | | | | | |
| 322/424/497/1907 | | | | | |
| miR-150/5127 | 6 | 13 | 202 | 0.00028 | 1.460568 |
| miR-320abcd/4429 | 7 | 22 | 557 | 0.00176 | 1.425897 |
| miR-495/1192 | 6 | 26 | 634 | 0.00047 | 1.41558 |
| miR-1ab/206/613 | 6 | 24 | 575 | 0.00054 | 1.403521 |
| miR-153 | 6 | 22 | 508 | 0.00064 | 1.388764 |
| miR-181abcd/4262 | 6 | 31 | 844 | 0.00069 | 1.38223 |
| miR-23abc/23b-3p | 7 | 28 | 809 | 0.00298 | 1.380157 |
| miR-221/222/222ab/ | 7 | 14 | 307 | 0.00344 | 1.367688 |
| 1928 | | | | | |
| miR-182 | 8 | 25 | 792 | 0.01507 | 1.364377 |
| miR-149 | 6 | 16 | 325 | 0.00098 | 1.351755 |
| miR-128/128ab | 8 | 23 | 726 | 0.01991 | 1.340186 |
| miR-17/17-5p/20ab/ | 8 | 26 | 853 | 0.02052 | 1.337565 |
| 20b-5p/93/106ab/ | | | | | |
| 427/518a-3p/519d | | | | | |
| miR-218/218a | 7 | 23 | 649 | 0.0051 | 1.333486 |
| miR-96/507/1271 | 8 | 23 | 732 | 0.02176 | 1.332468 |
| miR-374ab | 6 | 20 | 471 | 0.00126 | 1.329926 |
| miR-410/344de/ | 6 | 20 | 480 | 0.00148 | 1.315948 |
| 344b-1-3p | | | | | |
| miR-139-5p | 6 | 13 | 254 | 0.00176 | 1.300897 |
| miR-29abcd | 7 | 24 | 721 | 0.01016 | 1.273621 |
| miR-132/212/212- | 6 | 14 | 301 | 0.00265 | 1.265351 |
| 3p | | | | | |
| miR-375 | 6 | 10 | 179 | 0.00323 | 1.248159 |
| miR-145 | 6 | 20 | 517 | 0.00396 | 1.230461 |

In Table 2, this spanning score considers (a) the fraction of network clusters targeted by each miRNA and (b) the hypergeometric p-value of the overlap of the target pool of the miRNA with the expanded PH network. P-values were normalized by the theoretical maximum p-value, defined as the reciprocal of the number of simulations used to estimate the distribution (in this case, 100,000 simulations were used).

TABLE 3
PH-relevant targets of the miR-130/301 family ranked
according to their target spanning score.

| | | Normalized | | |
|---|---|---|---|---|
| | p- | Betweenness | Interacting | Overall |
| Gene | value | Centrality Score | Clusters | Score |
| SMAD4 | 0.0010 | 0.012537 | 7 | 1.637537 |
| ESR1 | 0.0115 | 0.020162 | 7 | 1.379987 |
| SMAD5 | 0.0007 | 0.003846 | 4 | 1.292572 |
| MAPK1 | 0.0351 | 0.020854 | 7 | 1.259528 |
| PPARG | 0.0281 | 0.003426 | 6 | 1.141249 |
| TGFBR1 | 0.0393 | 0.017649 | 6 | 1.119051 |
| SP1 | 0.1572 | 0.037286 | 7 | 1.113172 |
| AR | 0.0507 | 0.004285 | 6 | 1.078033 |
| PRKACB | 0.2429 | 0.010019 | 7 | 1.038662 |
| STAT3 | 0.3270 | 0.013249 | 7 | 1.009612 |
| NCOA3 | 0.0132 | 0.003565 | 4 | 0.973422 |
| TGFBR2 | 0.0481 | 0.015402 | 5 | 0.969866 |
| EDN1 | 0.1735 | 0.006163 | 6 | 0.946338 |
| TGFB2 | 0.0023 | 0.002050 | 2 | 0.911618 |
| PTEN | 0.2854 | 0.000788 | 6 | 0.886925 |
| BMPR1B | 0.0889 | 0.004058 | 4 | 0.766833 |
| CXCL12 | 0.3441 | 0.001432 | 5 | 0.742261 |
| HSPA8 | 0.6523 | 0.012750 | 5 | 0.684138 |
| ADCY1 | 0.0869 | 0.008517 | 3 | 0.648762 |
| BMPR2 | 0.1065 | 0.009363 | 3 | 0.627526 |
| PDE5A | 0.1055 | 0.003419 | 3 | 0.622606 |
| PDK1 | 0.6186 | 0.004293 | 4 | 0.556440 |
| SNX2 | 0.0634 | 0.000022 | 2 | 0.549500 |
| PDGFRA | 0.1024 | 0.001626 | 2 | 0.499051 |
| ZEB1 | 0.5120 | 0.000079 | 3 | 0.447762 |
| ROCK2 | 0.2248 | 0.000918 | 2 | 0.412969 |
| ARHGEF12 | 0.4499 | 0.000086 | 2 | 0.336807 |
| TRPC3 | 0.4569 | 0.000583 | 1 | 0.210628 |

In Table 3, this score considers (a) the normalized shortest path betweenness centrality score of the gene, (b) the fraction of network clusters with which the gene interacts, and (c) the hypergeometric p-value for the overlap of the gene's first degree neighborhood with the miR-130/301 target pool. P-values were normalized by the theoretical maximum p-value, defined as the reciprocal of the number of simulations used to estimate the distribution (in this case, 100,000 simulations were used). Betweenness centrality scores were normalized by the theoretical maximum score, defined as ½(N−1)(N−2), where N is the number of nodes in the network.

Analysis of Network Architecture Predicts Important Interconnections of miR-130/301 with Downstream miRNA-Gene Target Pathways in PH:

To predict important target genes, subordinate miRNAs, and related downstream pathways of miR-130/301 that may be the most influential in the progression of PH, we performed three additional analyses to interrogate, as broadly as possible, the distinct mechanisms by which this miRNA family could exert its global control of PH (FIG. 1D). First, to discern which of the miR-130/301 targets likely carry the broadest influence of the PH network, we ranked each miR-130/301 target based on architectural features representing the span of its network influence and the robustness of its association with the miR-130/301 family (Table 3). We refer to these features together as the “target spanning score,” incorporating: (a) the number of clusters in which the target had at least one first-degree interactor, (b) a hypergeometric p-value for the overlap of the target's first-degree neighborhood and the miR-130/301 predicted target pool, and (c) the target's shortest path betweenness centrality score, which measures the number of pairwise shortest paths between network nodes that require this target gene as a bridge. The top-ranked targets would therefore be expected to be highly influential in the context of PH (a high betweenness centrality score and a large number of interacting clusters) and highly sensitive to the regulatory effects of the miR-130/301 family (carrying a first-degree neighborhood of genes targeted multiple times by this miRNA family). The transforming growth factor/bone morphogenetic protein (TGF/BMP) pathway members SMAD4 and SMAD5 as well as the peroxisome proliferator-activated receptor gamma (PPARγ) were included among the top five genes. Importantly, their rankings together were consistent with prior reports linking upstream BMP signaling to PPARγ and PASMC proliferation (19) and other reports linking PPARγ directly to PH development (20-22). However, the full spectrum of the regulation and downstream actions of PPARγ in PH remains undescribed (23). Thus, we chose to focus further on the downstream biology of PPARγ in relation to the miR-130/301 family which seemed most relevant for an in-depth network-based interrogation.

Second, we reasoned that, regardless of its status as a direct target of the miR-130/301 family, a highly influential gene in the expanded PH network with a wide sphere of network influence should share functional overlap with the miR-130/301 family which regulates the network on a similarly broad scale. Thus, to cast an even wider net for capturing all genes highly active in the expanded PH network as a whole, we ranked all nodes in the expanded PH network according to their influence (“gene spanning score”; Table 4). Again, genes were scored on three architectural features reflecting their influence on the network as a whole: (a) their shortest path betweenness centrality score, (b) the number of architectural clusters in which they had at least one first-degree interactor, and (c) the degree of interactions by each gene in the network. The top-ranked gene was SRC, a non-receptor tyrosine kinase known to be associated with both STAT3 (the 18th ranked gene in this list) and miR-204 (the 4th ranked miRNA by miRNA spanning score, Table 2) in order to control PASMC proliferation in PH (7). Thus, while none of these factors have been linked to the miR-130/301 family, the substantial overlap of functional influence in the expanded PH network between STAT3/miR-204/SRC and the miR-130/301 family predicted their functional association.

TABLE 4
Top thirty expanded PH network genes ranked
according to their gene spanning score.

| | | Normalized | | |
|---|---|---|---|---|
| | | Betweenness | Number of | |
| | | Centrality | Clusters | Overall |
| Gene | Degree | Score | Targeted | Score |
| SRC | 77 | 0.065136 | 7 | 1.940136 |
| AKT1 | 63 | 0.032708 | 8 | 1.850889 |
| MAPK3 | 60 | 0.027858 | 8 | 1.807079 |
| SP1 | 66 | 0.037286 | 7 | 1.769428 |
| NFKB1 | 65 | 0.037156 | 7 | 1.756312 |
| EGFR | 64 | 0.037173 | 7 | 1.743342 |
| JUN | 55 | 0.023414 | 8 | 1.737699 |
| RELA | 62 | 0.024686 | 7 | 1.704881 |
| PRKCA | 59 | 0.046845 | 7 | 1.688079 |
| MAPK1 | 60 | 0.020854 | 7 | 1.675075 |
| PIK3R1 | 50 | 0.018902 | 8 | 1.668252 |
| GRB2 | 57 | 0.026861 | 7 | 1.642121 |
| PRKACA | 52 | 0.046974 | 7 | 1.597298 |
| SMAD2 | 53 | 0.023723 | 7 | 1.587034 |
| TP53 | 52 | 0.023825 | 7 | 1.574149 |
| SMAD3 | 51 | 0.025814 | 6 | 1.568022 |
| ESR1 | 47 | 0.020162 | 7 | 1.557499 |
| STAT3 | 45 | 0.013249 | 7 | 1.550586 |
| CAV1 | 47 | 0.018252 | 7 | 1.503641 |
| SMAD4 | 45 | 0.012537 | 7 | 1.471952 |
| CALM1 | 42 | 0.029327 | 7 | 1.449781 |
| MAPK8 | 43 | 0.014189 | 7 | 1.447630 |
| SMAD1 | 42 | 0.016646 | 7 | 1.437101 |
| ACTB | 38 | 0.015426 | 7 | 1.383932 |
| RHOA | 46 | 0.028132 | 6 | 1.375534 |
| CTNNB1 | 47 | 0.012331 | 6 | 1.372721 |
| EP300 | 45 | 0.016247 | 6 | 1.350662 |
| FOS | 44 | 0.028030 | 6 | 1.349459 |
| UBC | 35 | 0.013388 | 7 | 1.342933 |
| YWHAZ | 34 | 0.021789 | 7 | 1.338347 |

In Table 4, this score which considers (a) the degree of the gene in the expanded PH network, (b) its shortest path betweenness centrality score in the expanded PH network, and (c) the fraction of network clusters with which it interacts. Degrees were normalized by the theoretical maximum degree, defined as N−1, where N is the number of nodes in the network. Betweenness centrality scores were normalized by the theoretical maximum score, defined as ½(N−1)(N−2), where N is the number of nodes in the network.

Finally, to capture additional miRNAs that may be associated with the miR-130/301-dependent regulation of PH, we ranked all conserved miRNAs represented in the TargetScan algorithm according a hypergeometric analysis of the overlap of their PH-relevant target pools with the PH-relevant target pool of the miR-130/301 family (“shared miRNA influence score”; Table 5). The top-ranked set of miRNAs included miR-424, a miRNA also ranked highly (top 10) by miRNA spanning score (Table 2). Thus, although miR-424 had not previously been associated with the miR-130/301 family, these rankings indirectly connected these miRNAs by their broad spheres of influence in PH. Specifically, along with miR-503, miR-424 is known to be dysregulated in PH by alterations in the peptide ligand apelin (APLN), thus inciting up-regulation of fibroblast growth factor-2 (FGF2) in PAECs (8). Furthermore, while itself never previously connected to the miR-130/301 family, APLN has been associated with both miR-424 and miR-503 (8) as well as PPARγ in PAECs (24). Consequently, the generation of the expanded PH network coupled with computational analyses of network architecture not only predicted the broad influence of the miR-130/301 family in PH pathogenesis but also provided a detailed map of integrated miRNA-gene target pathways by which this miRNA family may exert its influence.

TABLE 5
Top thirty miRNA families ranked according
to their shared miRNA influence score

| | | Targets and | |
|---|---|---|---|
| | | 1st Degree | Overlap with |
| | p- | Interactors | miR-130/301 |
| miRNA | value | in PH Network | Target Pool |
| miR-15abc/16/16abc/195/ | 0.00507 | 155 | 24 |
| 322/424/497/1907 | | | |
| miR-30abcdef/30abe- | 0.0057 | 145 | 23 |
| 5p/384-5p | | | |
| miR-23abc/23b-3p | 0.0068 | 157 | 24 |
| miR-197 | 0.0053 | 67 | 14 |
| miR-340-5p | 0.00629 | 168 | 25 |
| miR-141/200a | 0.00631 | 127 | 21 |
| miR-196abc | 0.00634 | 68 | 14 |
| miR-140/140-5p/876- | 0.00638 | 83 | 16 |
| 3p/1244 | | | |
| miR-431 | 0.007 | 68 | 14 |
| miR-342-3p | 0.0074 | 69 | 14 |
| miR-142-3p | 0.00823 | 70 | 14 |
| miR-346 | 0.0083 | 85 | 16 |
| miR-486-5p/3107 | 0.0096 | 71 | 14 |
| miR-223 | 0.01057 | 96 | 17 |
| miR-146ac/146b-5p | 0.01065 | 50 | 11 |
| miR-33ab/33-5p | 0.01074 | 122 | 20 |
| miR-490-3p | 0.01109 | 43 | 10 |
| miR-758 | 0.01141 | 72 | 14 |
| miR-1ab/206/613 | 0.01154 | 162 | 24 |
| miR-873 | 0.012 | 80 | 15 |
| miR-384/384-3p | 0.01234 | 97 | 17 |
| miR-876-5p/3167 | 0.0128 | 89 | 16 |
| miR-224 | 0.01369 | 81 | 15 |
| miR-143/1721/4770 | 0.01374 | 115 | 19 |
| miR-376c/741-5p | 0.01513 | 90 | 16 |
| miR-190/190ab | 0.01521 | 82 | 15 |
| miR-132/212/212-3p | 0.01553 | 116 | 19 |
| miR-192/215 | 0.01622 | 20 | 6 |
| miR-326/330/330-5p | 0.01688 | 83 | 15 |
| miR-148ab-3p/152 | 0.01733 | 117 | 19 |

In Table 5, this score considers the hypergeometric p-value of the overlap of their wider target pool (defined as their pool of direct targets and all first degree interactors of those targets), with the direct target pool of the miR-130/301 family. P-values were normalized by the theoretical maximum p-value, defined as the reciprocal of the number of simulations used to estimate the distribution (in this case, 100,000 simulations were used).

The miR-130/301 Family is Up-Regulated by Multiple Triggers of PH, Including Hypoxia Via a Dependence on HIP-2α and POU5F1/OCT4:

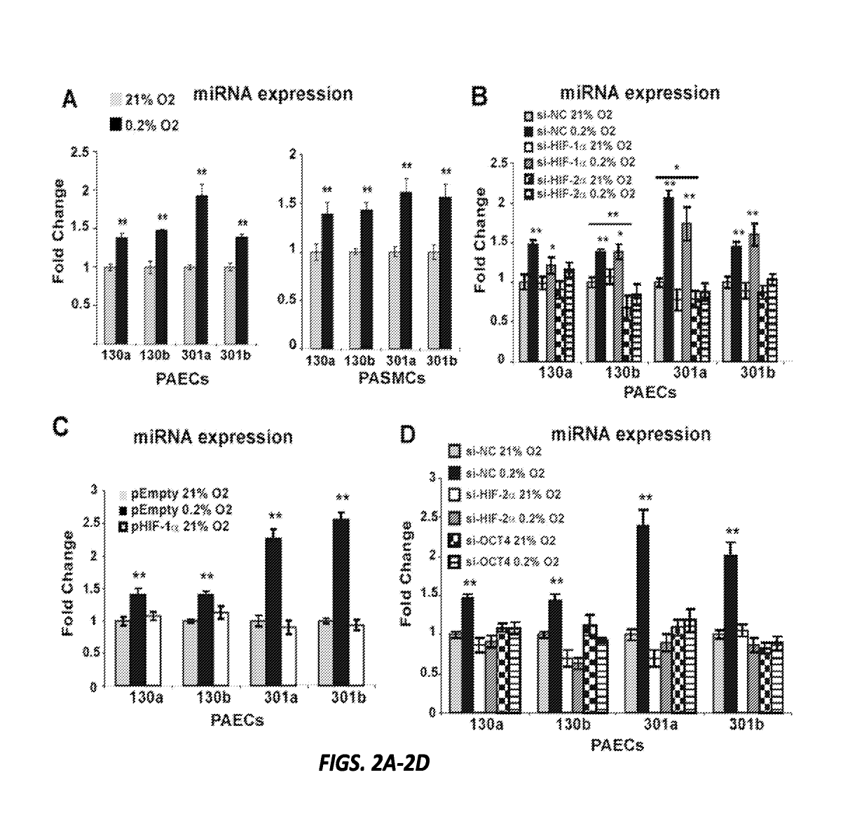

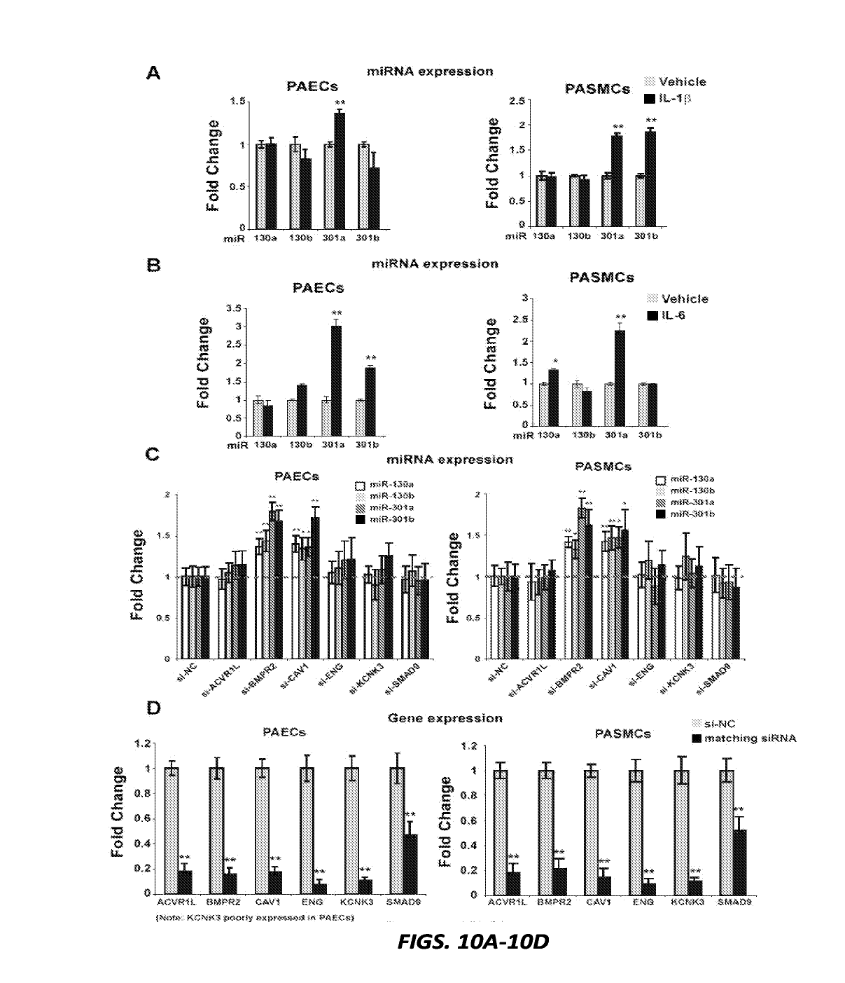

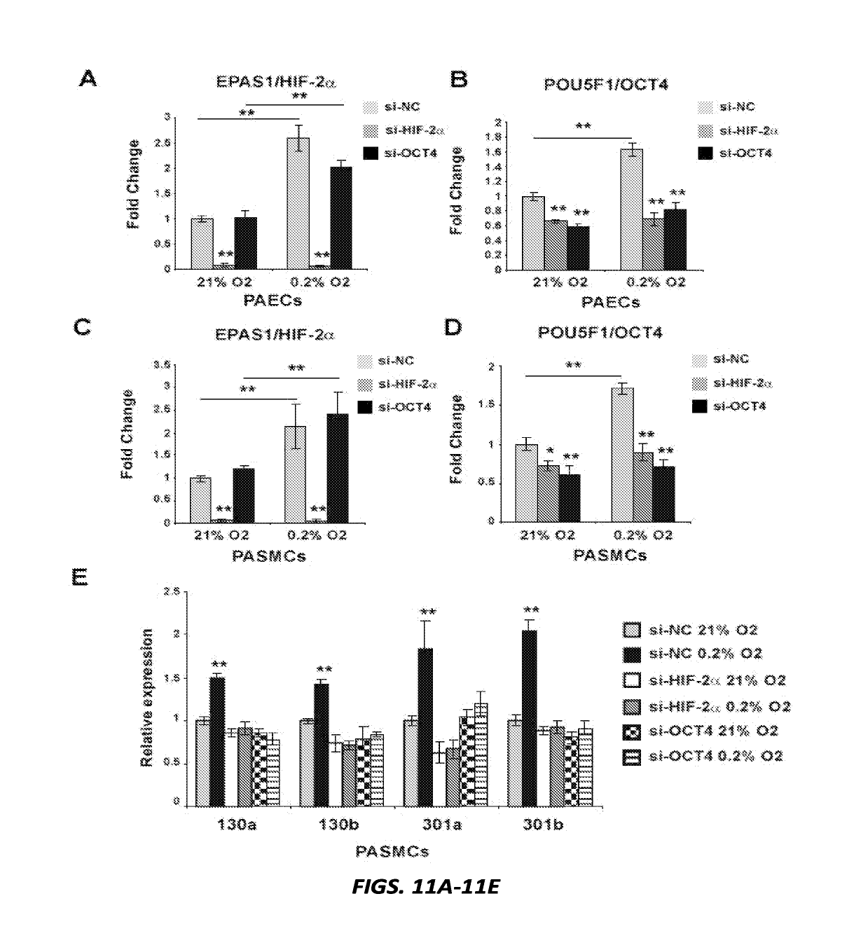

To begin to validate this molecular model, we wanted to determine if the miR-130/301 family is coordinately regulated by known triggers of PH. First, we investigated whether genes previously linked to hereditary PAH [as reviewed by (25)] may regulate this miRNA family. In both human PAECs and PASMCs (FIGS. 10C-10D), we found that siRNA-mediated knockdown of BMPR2 or CAV1 increased miR-130/301 expression, while inhibition of ALK1, ENG, KCNK3, and SMAD9 had negligible effects on this miRNA family. Next, we found that multiple acquired PH triggers, including hypoxia (FIG. 2A) and inflammatory cytokines, interleukin-1β (IL-1β, FIG. 10A) and interleukin-6 (IL-6, FIG. 10B), up-regulate miR-130/301 expression as a whole but with some specific differences in family member profile in each context. Consistent with our results (FIG. 10; Table 2), some miR-130/301 family members have been reported to be transcribed by a NF-κB-dependent mechanism during inflammatory cytokine stimulation (13). Alternatively, we found that knockdown of a master transcription factor of hypoxia, HIF-2 abut not HIF-1α prevented the increase of these miRNAs in hypoxia (FIG. 2B). In corroboration, forced expression of HIF-1α was insufficient to up-regulate any miR-130/301 family members (FIG. 2C). No identifiable binding sites were predicted for HIF-2α in the promoter region of any miR-130/301 family members (Table 6). However, a putative binding sequence was predicted in all family member promoter sites for the transcription factor POU5F1/OCT4, a factor known to be modulated by HIF-2α but not HIF-1α (26) and known to be up-regulated in PAH (27). Consequently, we found that POU5F1/OCT4 was up-regulated in hypoxia and was dependent on HIF-2α in PAECs and PASMCs (FIGS. 11A-11D). Furthermore, siRNA knockdown of POU5F1/OCT4 inhibited miR-130/301 induction by hypoxia in PAECs (FIG. 2D) and PASMCs (FIG. 11E), thus establishing the critical importance of the HIF-2α-POU5F1/OCT4 regulatory axis in miR-130/301 expression. Moreover, taken together, these data demonstrated the broad convergence of both genetically associated and acquired PH triggers on coordinated regulation of this key miRNA family.

TABLE 6
Transcription factors predicted to bind the promoters
of the different members of miR-130/301 family

| miR-130a | miR-130b | miR-301a | miR-301b | |
|---|---|---|---|---|
| Transcription | Transcription | Transcription | Transcription | |
| Factors | Factors | Factors | Factors | |
| (From | (From | (From | (From | |
| ChIPBase) | ChIPBase) | ChIPBase) | ChIPBase) | Shared |
| BAF155 | AP-2alpha | AP-2alpha | AP-2alpha | BAF155 |
| BAF170 | AP-2gamma | AP-2gamma | AP-2gamma | CDX2 |
| CDX2 | AR | BAF155 | AR | CEBPB |
| CEBPB | BAF155 | BAF170 | BAF155 | ETS1 |
| CTCF | BRG1 | BATF | BRG1 | HEY1 |
| ETS1 | CBP | BDP1 | CBP | HNF4A |
| FOXH1 | CDX2 | BRF1 | CDX2 | JUN |
| FOXP2 | CEBPA | BRG1 | CEBPA | NFKB |
| GATA6 | CEBPB | CBP | CEBPB | NFYB |
| GR | CTCF | CDX2 | CTCF | OCT4 |
| HEY1 | E2F4 | CEBPB | E2F4 | PU.1 |
| HNF4A | E2F6 | E2F1 | E2F6 | TAF1 |
| JUN | EBF | E2F4 | EBF | YY1 |
| NANOG | ERalpha | E2F6 | ERalpha | |
| NFYB | ERG | ERalpha | ERG | |
| NFKB | ETS1 | ERG | ETS1 | |
| NRSF | FOS | ETS1 | FOS | |
| OCT-4 | FOSL2 | FOS | FOSL2 | |
| EP300 | FOXP2 | GABP | FOXP2 | |
| P63 | GATA1 | GATA6 | GATA1 | |
| PU.1 | GATA2 | GTF2B | GATA2 | |
| RAD21 | HEY1 | HA-E2F1 | HEY1 | |
| RPC155 | HFN4A | HEY1 | FMF4A | |
| RXRA | INI1 | FMF4A | INI1 | |
| SMAD2/3 | IRF4 | INI1 | IRF4 | |
| SMAD3 | JUN | IRF4 | JUN | |
| SMAD4 | JUND | JUN | JUND | |
| TAF1 | MAX | JUND | MAX | |
| YY1 | MED12 | MAX | MED12 | |
| | MYC | MED12 | MYC | |
| | NFYA | MYC | NFYA | |
| | NFYB | NANOG | NFYB | |
| | NFKB | NFYA | NFKB | |
| | NRSF | NFYB | NRSF | |
| | OCT-4 | NFKB | OCT-4 | |
| | PAX5-C20 | OCT-4 | PAX5-C20 | |
| | POU2F2 | PAX5-C20 | POU2F2 | |
| | PU.1 | PAX5-N19 | PU.1 | |
| | RAD21 | PBX3 | RAD21 | |
| | SETDB1 | POU2F2 | SETDB1 | |
| | SIN3AK-20 | PU.1 | SIN3AK-20 | |
| | SIRT6 | RPC155 | SIRT6 | |
| | SIX5 | SIN3AK-20 | SIX5 | |
| | SMAD3 | SP1 | SMAD3 | |
| | SP1 | SPDEF | SP1 | |
| | SREBP1 | SREBP1 | SREBP1 | |
| | STAT1 | SREBP2 | STAT1 | |
| | STAT2 | STAT1 | STAT2 | |
| | TAF1 | TAF1 | TAF1 | |
| | TCF12 | TCF12 | TCF12 | |
| | TCF7L2 | TCF7L2 | TCF7L2 | |
| | TR4 | USF1 | TR4 | |
| | YY1 | YY1 | YY1 | |
| | ZNF263 | | ZNF263 | |

In Table 6, of twelve regulators with binding sites shared among promoters of all miR-130/301 members, only POU5F1/OCT4 (OCT4) was previously found to be a transcriptional target of HIF-2α, thus singling it out as a potential mediator of HIF-2α-dependent up-regulation of the miR-130/301 family.

The miR-130/301 Family is Up-Regulated in PH In Vivo in Animals and Humans:

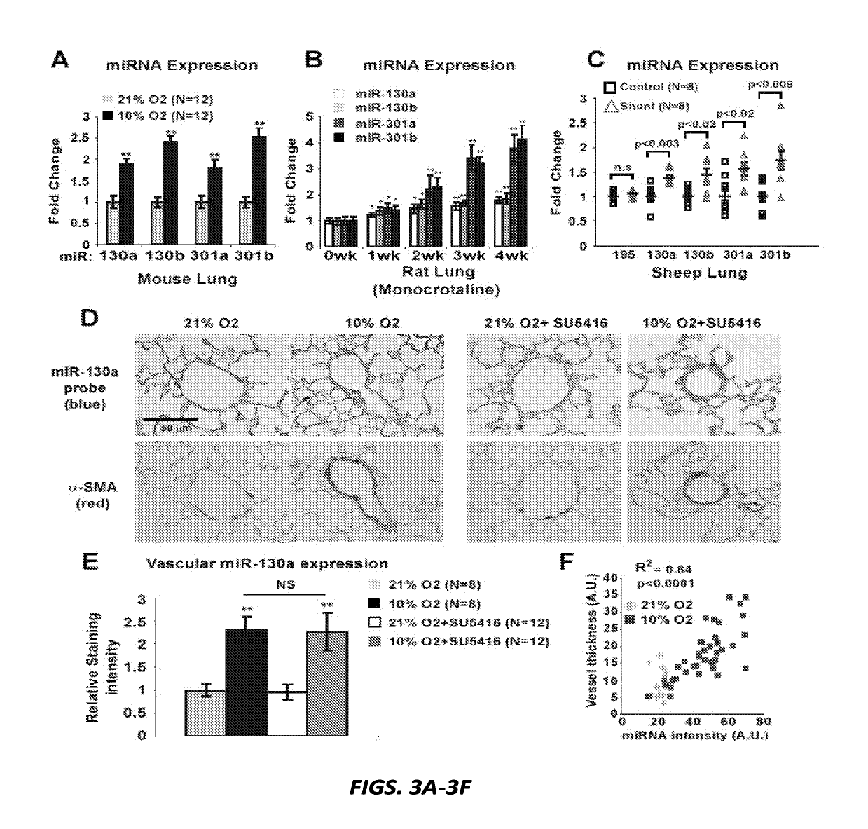

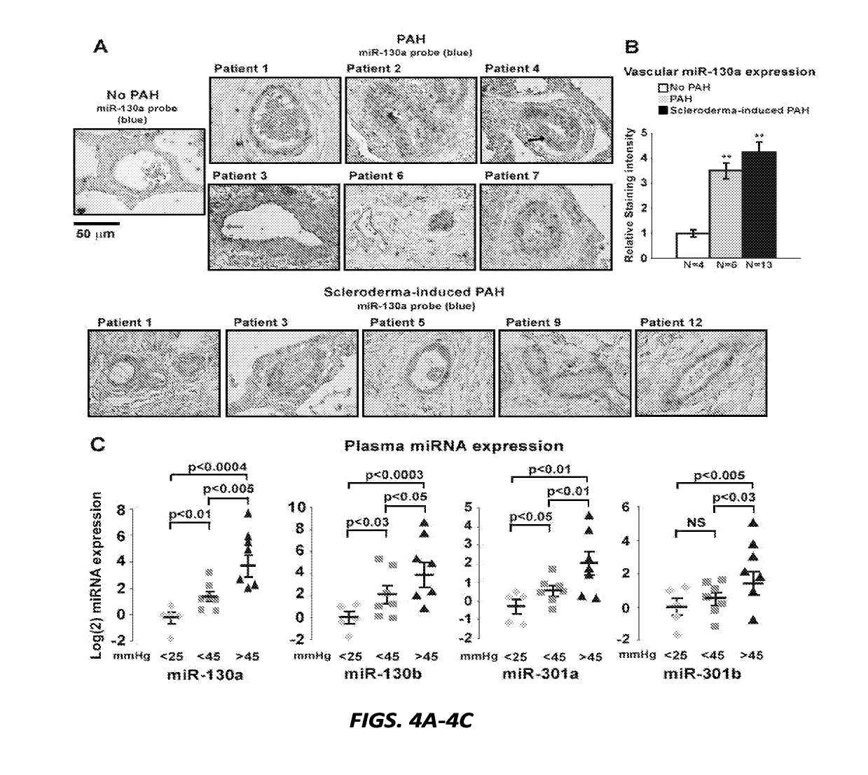

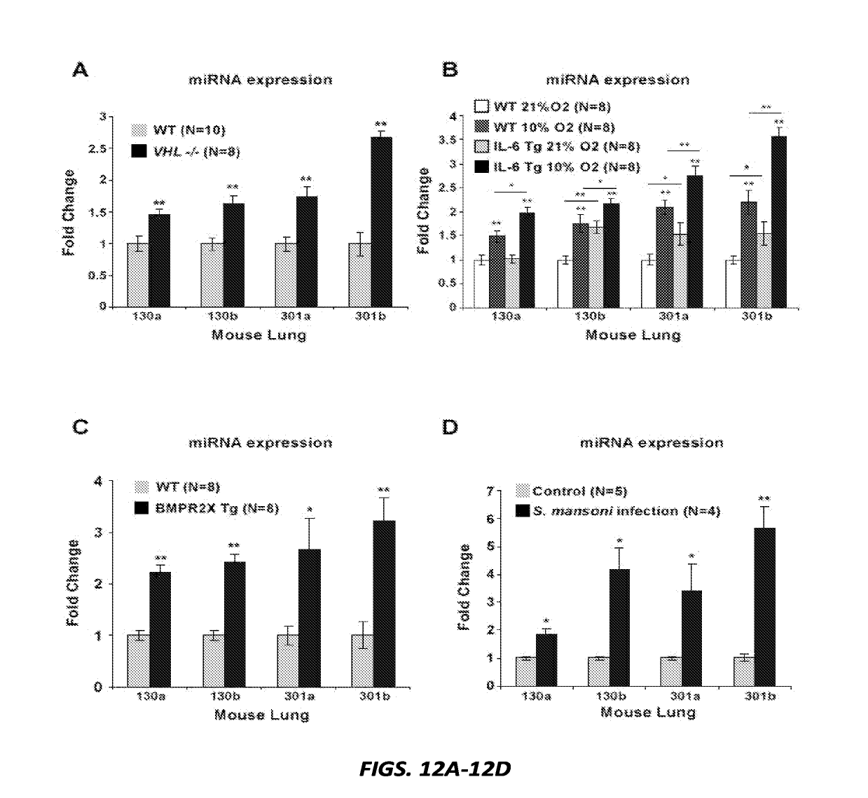

Again correlating with our network-based predictions of the broad actions of miR-130/301 in PH, we found consistent up-regulation of this miRNA family in whole lung tissue of eight disparate, yet well-established, animals models of experimental PH (FIGS. 3A-3C; FIGS. 12A-12D). These included hypoxia-driven models [mice treated with chronic hypoxia alone, mice treated with chronic hypoxia and the VEGF receptor antagonist SU5416 (28), and VHL-null mice (9)]; inflammatory-driven models [transgenic IL-6 mice (29), monocrotaline-treated rat, and S. mansoni-infected mice (30)]; a genetic model of BMPR2 deficiency [transgenic mice overexpressing a dominant-negative mutant BMPR2 (BMPR2X) (31)]; and a shunt model of congenital heart disease [juvenile lambs with a surgically placed PA-aortic shunt at late gestation (32)]. In situ staining also revealed increased miR-130a, the most abundantly expressed of the miRNA family members (data not shown), throughout the vascular wall of the small (<100 μm diameter) pulmonary arterioles of mice suffering from PH induced by chronic hypoxia+SU5416 as compared with normoxic mice (FIGS. 3D-3E). Notably, pulmonary vascular staining intensity for miR-130a linearly correlated with muscular arteriolar wall thickness (α-smooth muscle actin stain) in these PH mice (FIG. 3F). miR-130a expression was also up-regulated in the small diseased pulmonary vessels in human lung afflicted with severe forms of PH (pulmonary arterial hypertension or PAH) as compared with non-diseased human tissue (FIGS. 4A-4B). Members of the miR-130/301 family were also significantly elevated in plasma sampled adjacent to the main pulmonary artery from PH individuals (mean pulmonary arterial pressures, mPAP>25 mmHg) as compared with non-PH individuals (mPAP<25 mmHg, FIG. 4C). Further stratification of PH individuals based on pulmonary arterial pressures (e.g., moderate elevation of mPAP between 25 to 45 mmHg as compared with elevation of mPAP>45 mmHg) revealed an increasing level of miRNA family member expression with hemodynamic severity. Thus, in animal models and human examples of PH in vivo, expression of the miR-130/301 family is consistently elevated in pulmonary tissue and small diseased pulmonary vessels—a pattern that positively correlates with histologic and hemodynamic severity of disease.

The miR-130/301 Family Regulates Pulmonary Vascular Cell Proliferation Via Repression of its Target PPARγ:

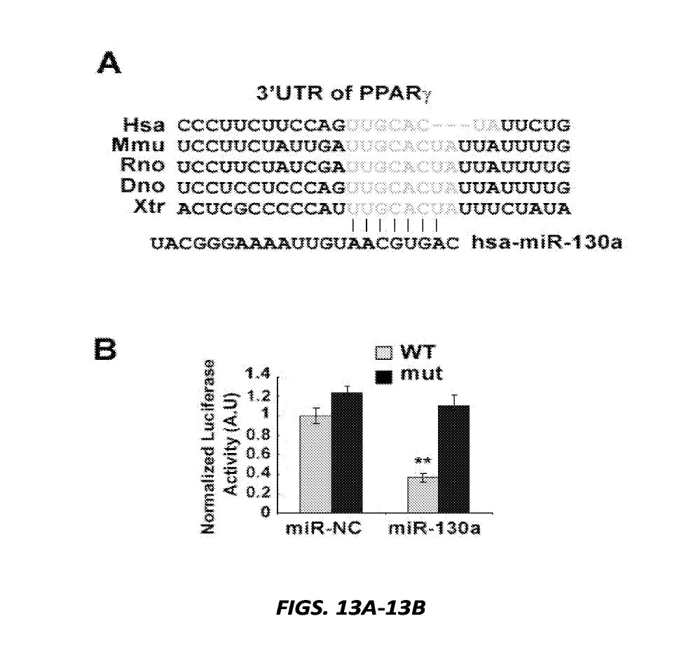

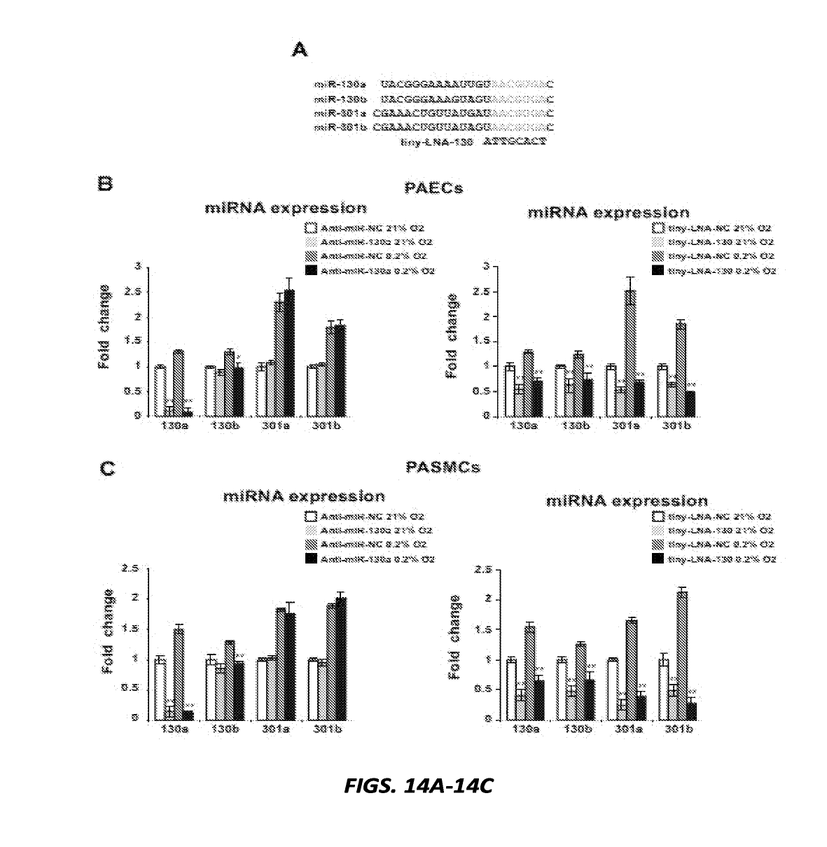

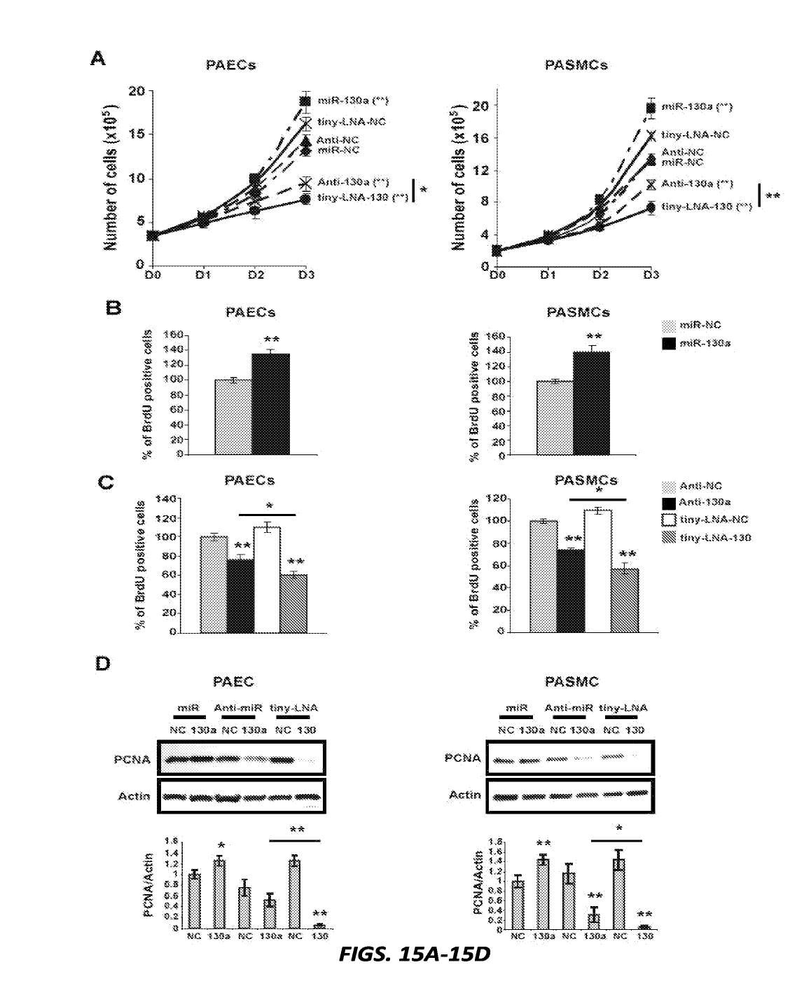

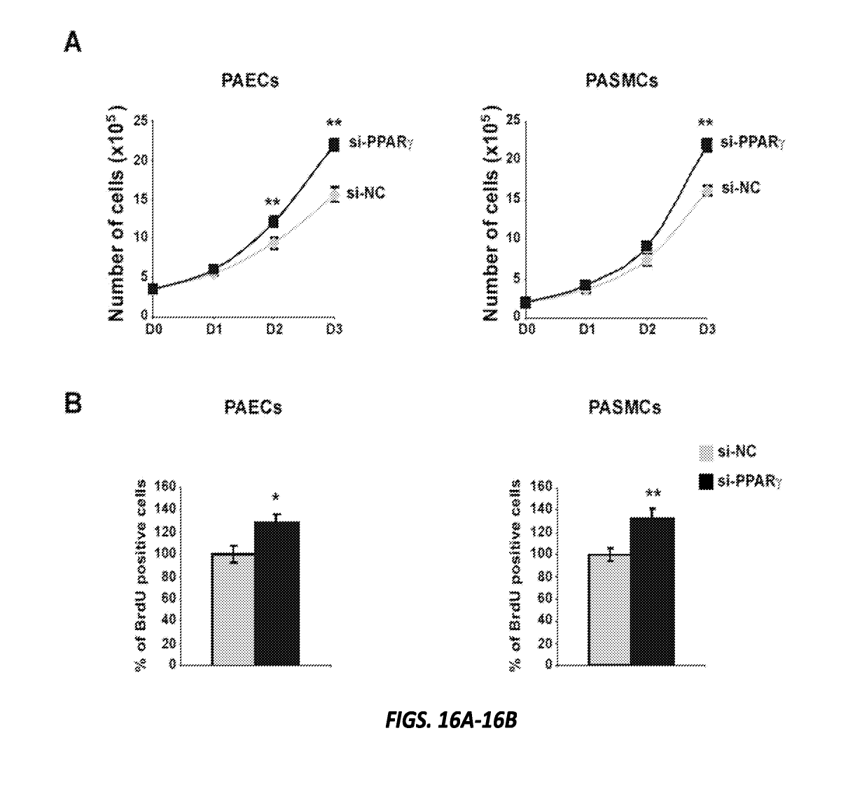

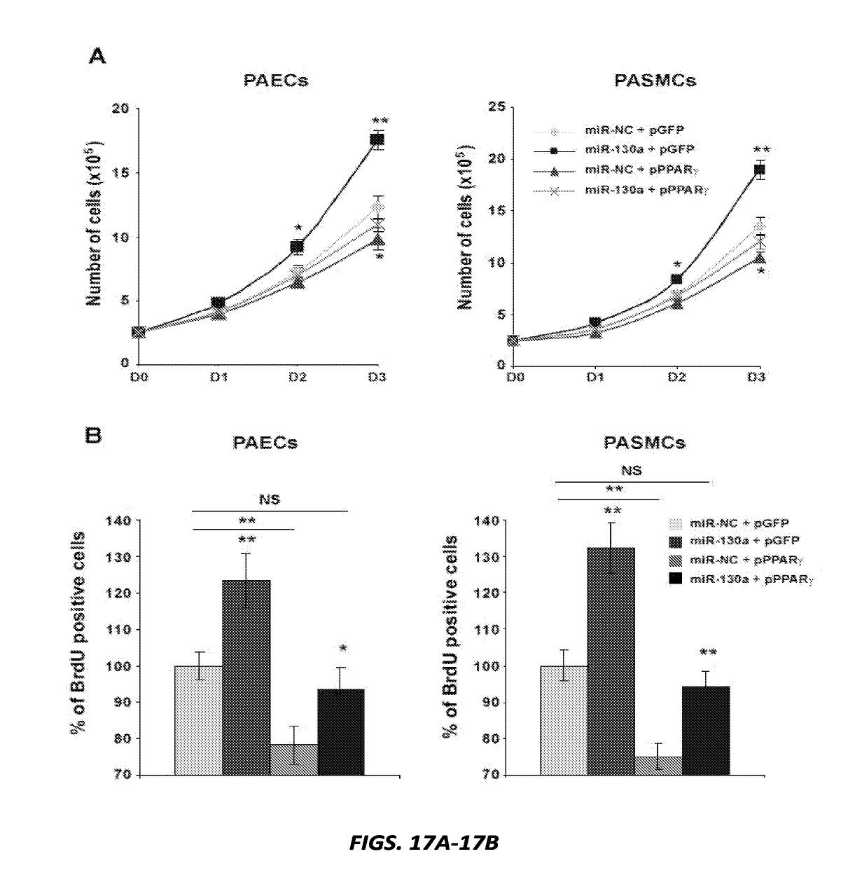

We next focused our attention on PPARγ, a direct target of the miR-130/301 family that was predicted by our network-based approach to mediate the broad actions of this miRNA family specifically in the PH network. Consistent with prior reports in different cellular contexts (33), we confirmed a sequence in the 3′ untranslated region of the PPARγ transcript as a direct binding target for miR-130a (FIG. 13). Forced expression of miR-130a in PAECs down-regulated PPARγ (FIGS. 5A-5B). Short locked nucleic acid oligonucleotides (e.g., “tiny-LNAs”) with antisense complementarity only to the seed sequence of this miRNA family (tiny-LNA-130, FIG. 14A) (34) were validated in PAECs and PASMCs as effective inhibitors of the entire miR-130/301 family (FIGS. 14B-14C). Tiny-LNA-130 increased PPARγexpression (FIGS. 5A-5B), notably to a greater extent than an inhibitor of miR-130a alone. As demonstrated by increased cell number, BrdU incorporation, and increased expression of the proliferation marker PCNA, forced expression of miR-130a increased cell proliferation in both PAECs and PASMCs (FIG. 15), which was phenocopied by siRNA knockdown of PPARγ (FIG. 16). Conversely, inhibition of the entire miR-130/301 family was substantially more effective in preventing proliferation than inhibition of miR-130a alone (FIG. 15). Importantly, forced expression of PPARγ in both cell types reversed the proliferative phenotype induced by miR-130a alone (FIG. 17). Thus, we confirmed that PPARγ carries an essential role in mediating the proliferative effects of the miR-130/301 family on pulmonary vascular cell types.

The miR-130/301-PPARγ Regulatory Axis Controls Context-Specific PAEC Proliferation and Apoptotic Signaling by Regulating Apelin, miR-424/503, and FGF2:

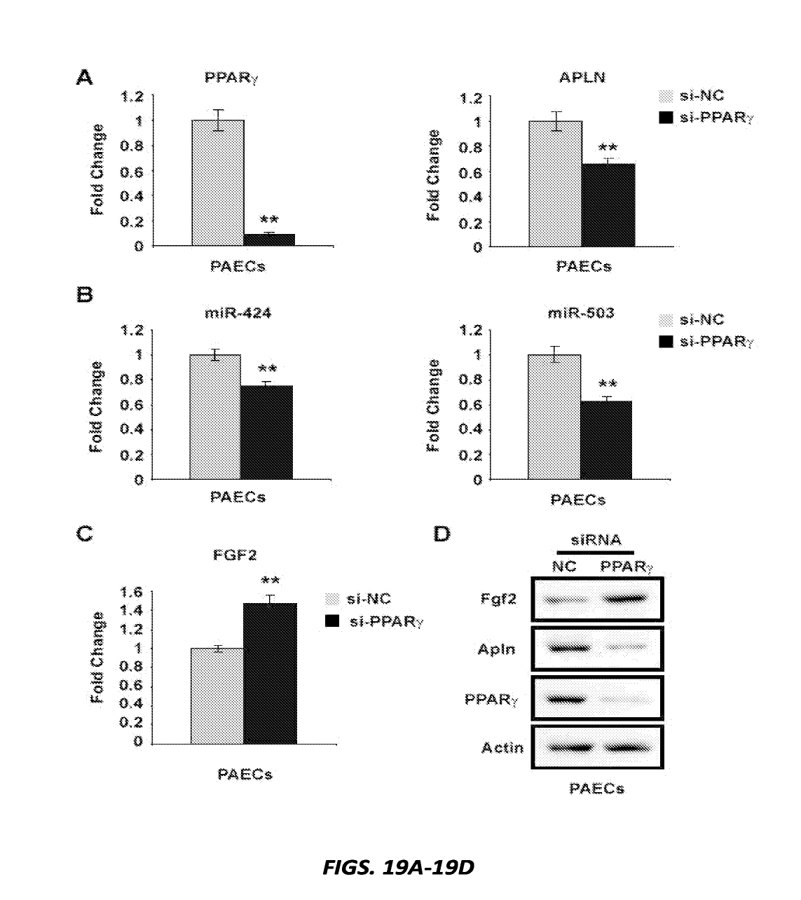

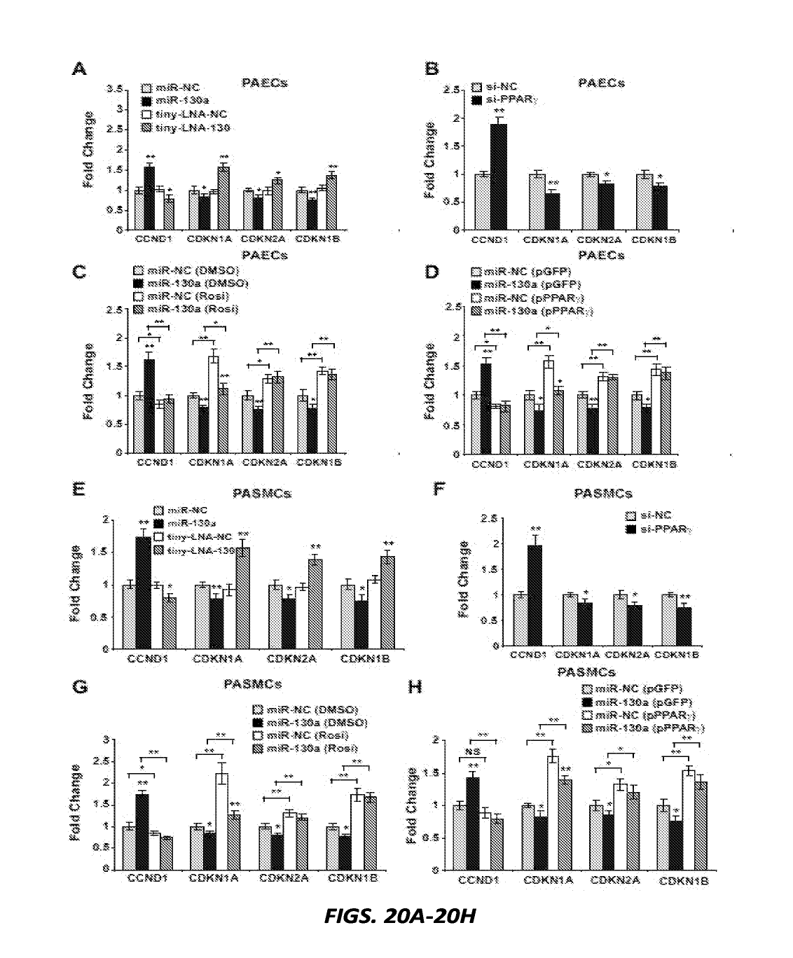

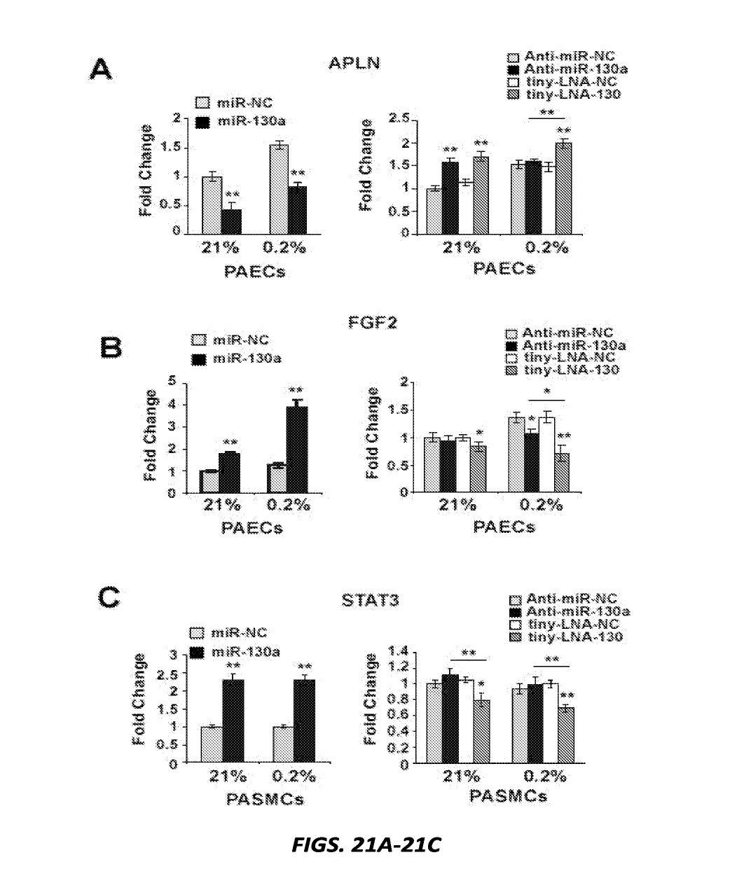

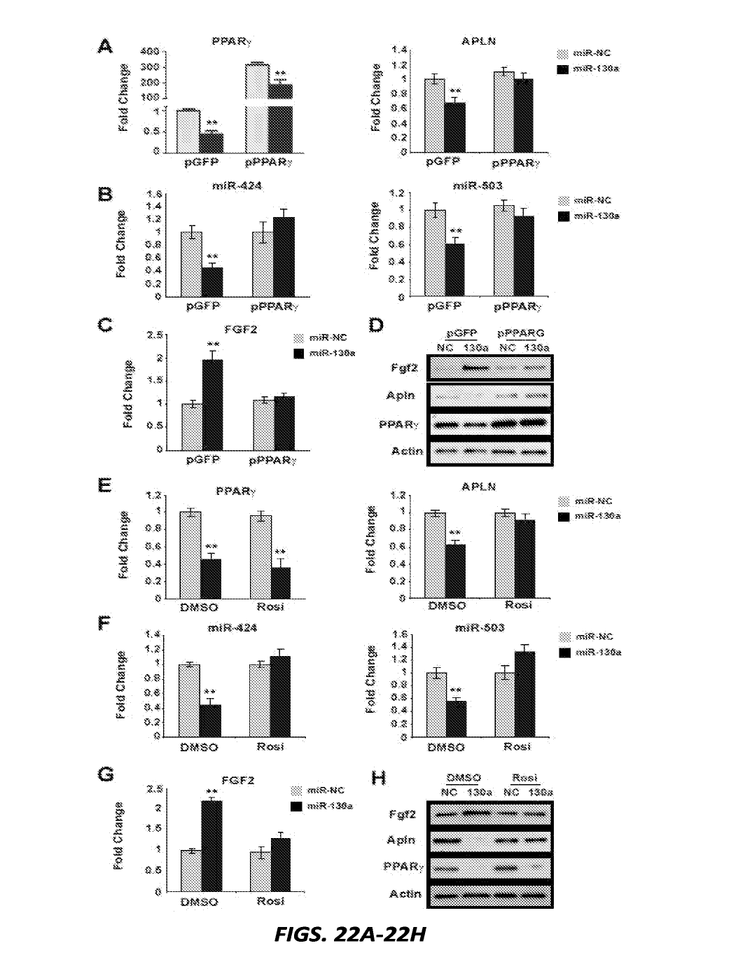

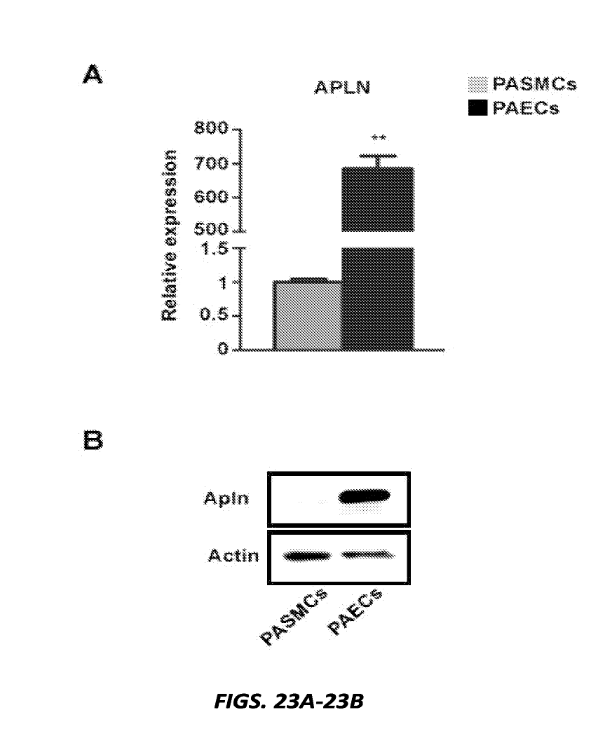

To further validate our in silico model of the miR-130/301 family, we aimed to determine whether a functional connection truly exists between the miR-130/301 family and the PH-specific pathway connecting apelin, miR-424/503, and FGF2. Because APLN expression is endothelial-specific (FIG. 23), we reasoned that this regulatory axis may be readily apparent in PAECs. In PAECs, forced expression of miR-130a (or siRNA knockdown of PPARγ, FIG. 19) down-regulated apelin, miR-424, and miR-503 (FIGS. 5A-5D and FIG. 21A), and up-regulated FGF2 (FIGS. 5A-5B and FIG. 21B). Converse regulation of this molecular pathway was observed by inhibition of the miR-130/301 family (FIGS. 5A-5D, FIG. 21)—effects that were again more robust than inhibition of miR-130a alone and effects that were apparent during both normoxia and hypoxia. Demonstrating the essential role of PPARγ in these responses, miR-130a-dependent gene alterations were rescued by restoring PPARγ function by either forced PPARγ expression (FIGS. 22A-22D) or pharmacologic PPARγ activation (rosiglitazone) (FIGS. 22E-22H). Notably, we found that a number of PPARγ-dependent target genes related to the cell cycle, such as CCND1, CDKN1A, CDKN2A, and CDKN2B (19, 35-37), were also modulated by manipulation of the miR-130/301 family or PPARγ (FIGS. 20A-20D). However, proving the critical role of miR-424/503 in this regulatory network, forced expression of miR-424 or miR-503 individually—and to a better extent when both miRNAs were expressed together—reversed the proliferative response to miR-130a (FIG. 5E). Thus, in PAECs, the miR-130/301-PPARγ regulatory axis controls cellular proliferation by repressing apelin and its subordinate miRNAs (miR-424/503, FIG. 5F), thus validating one arm of the PH-specific hierarchy of miRNA regulation predicted by network analysis.

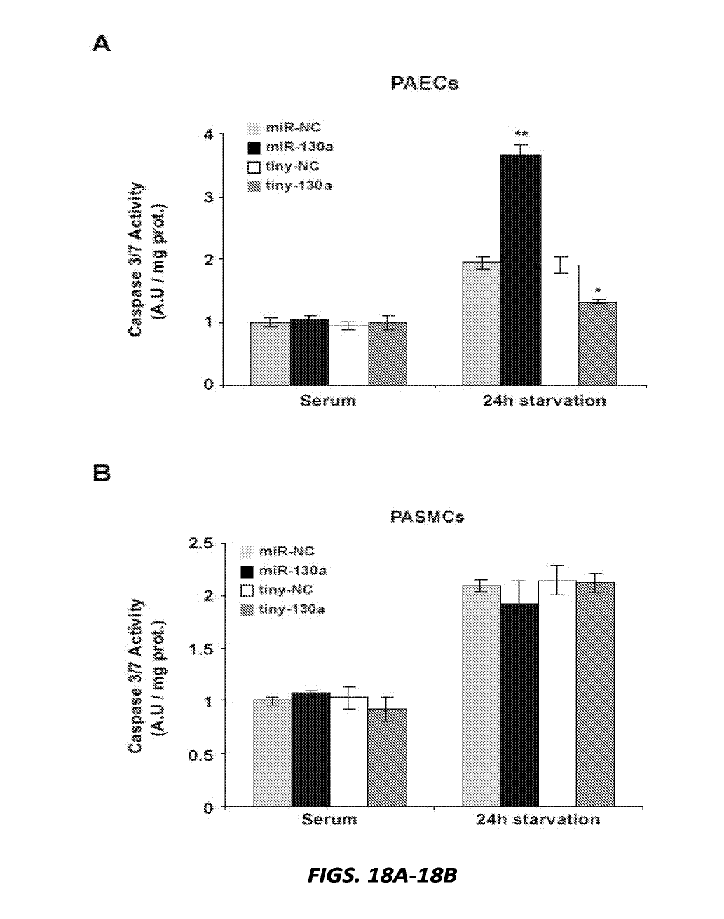

Beyond the network-based predictions regarding endothelial proliferation, we found that forced expression of miR-130a increased apoptotic caspase signaling in serum-starved cultured PAECs, while inhibition of miR-130/301 protected against these effects (FIG. 18A). These observations are consistent with prior observations of reduced APLN causing endothelial apoptosis (38) as an inciting pathogenic event to allow for selection of apoptosis-resistant PAECs in PAH (39). Notably, however, this miRNA family did not exert similar control over apoptotic activity in serum-starved PASMCs (FIG. 18B) nor were these actions evident in the presence of serum (FIG. 18A). Thus, this dual nature of miR-130/301 promoting context-specific apoptotic signaling and proliferation in PAECs indicates both the complexity and the substantially broad control over multiple disparate pathways converging to promote PH.

The miR-130/301-PPARγ Controls PASMC Proliferation Via Regulation of miR-204 and STAT3:

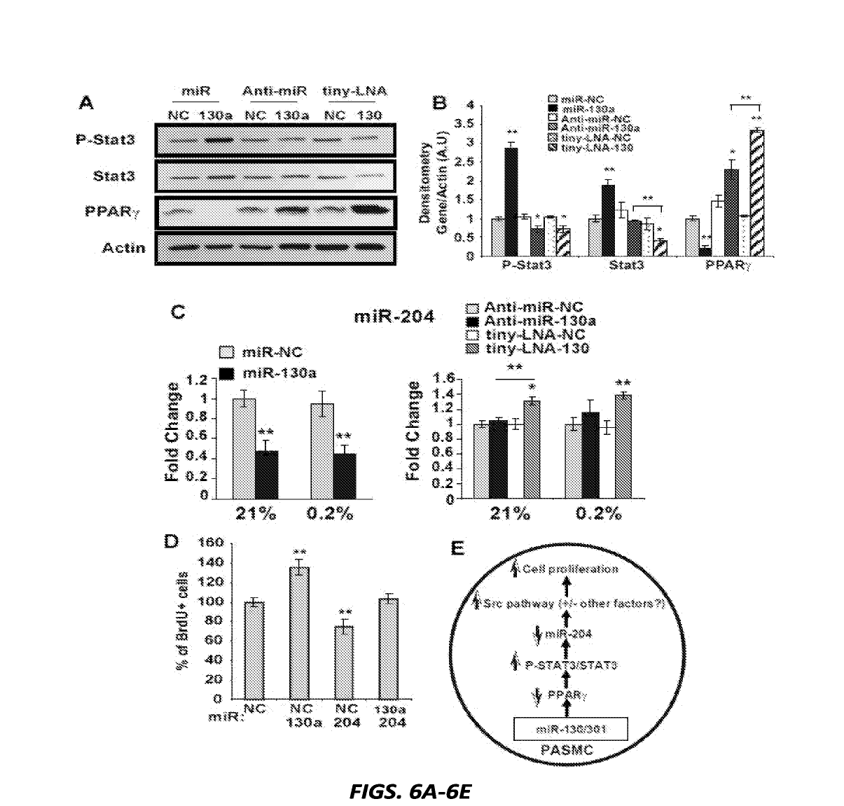

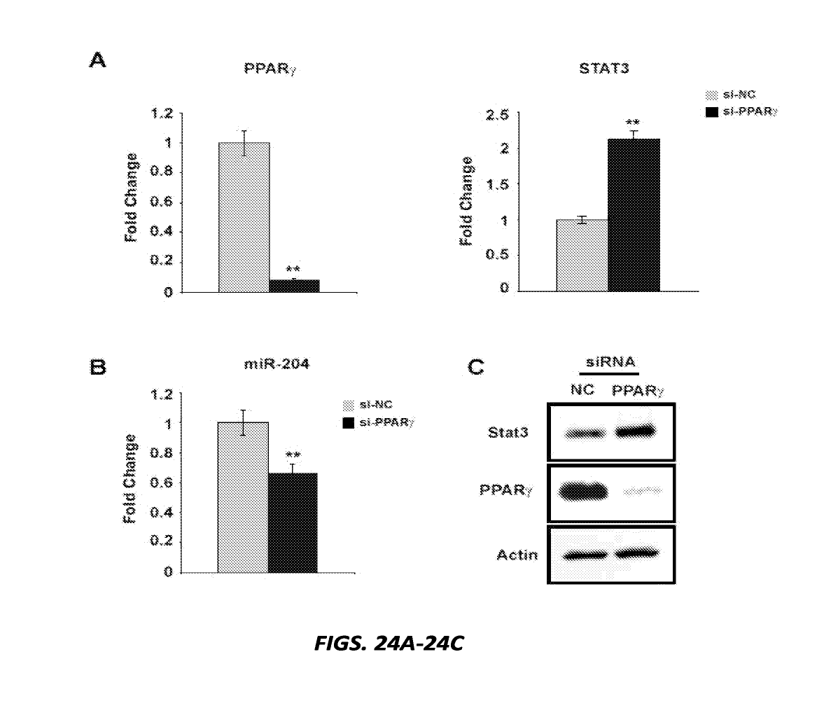

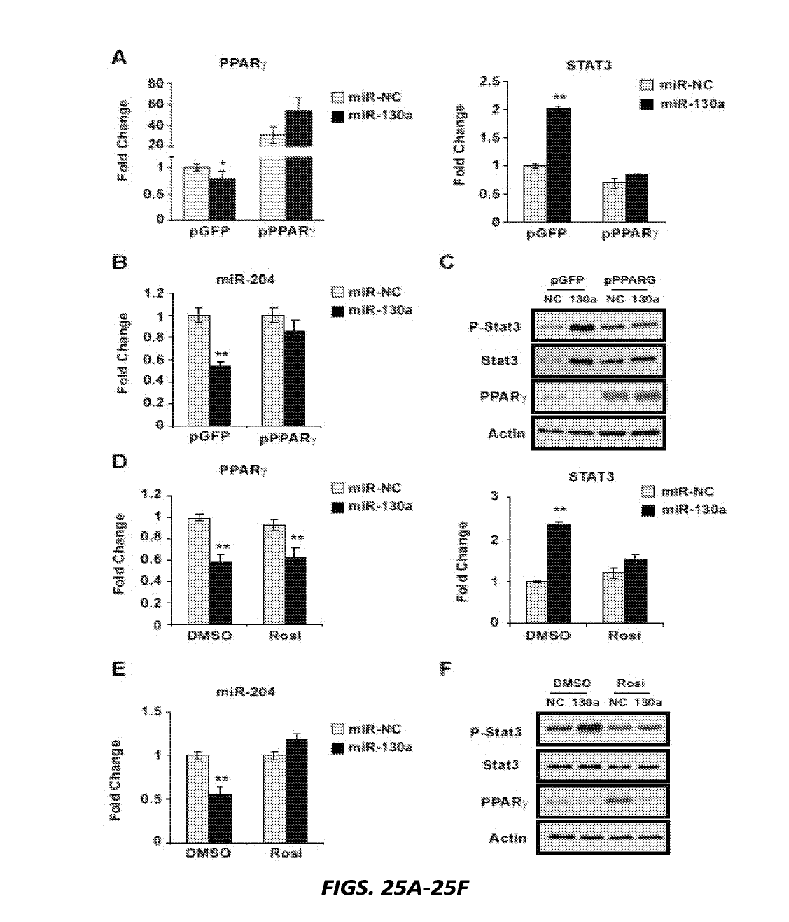

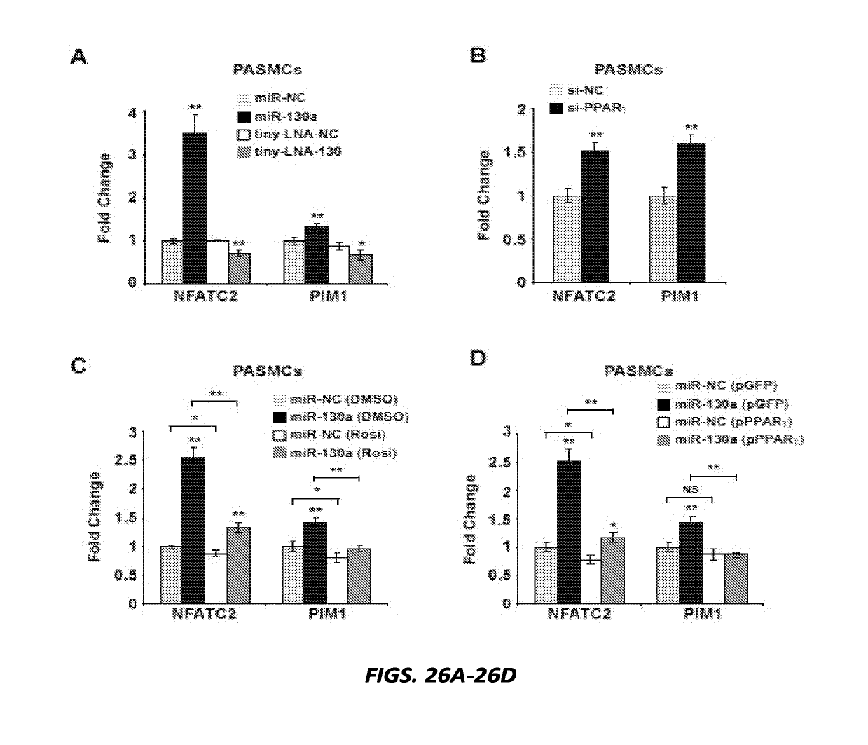

We then interrogated the functional relationship between the miR-130/301 family and the STAT3/miR-204/Src kinase pathway, as predicted by our network-based modeling. Given prior reports that PPARγ represses STAT3 transcription (40) and the localized actions of miR-204 in PASMCs (7), we further hypothesized that, in PASMCs, miR-130/301-dependent repression of PPARγ up-regulates STAT3 expression and/or activity and thus down-regulates miR-204. Correspondingly, in PASMCs, forced expression of miR-130a (or siRNA knockdown of PPARγ, FIG. 24) decreased PPARγ, accompanied by increased STAT3 expression and decreased miR-204 (FIGS. 6A-6C and FIG. 21C). As in PAECs, these expression changes were reversed by either forced expression of PPARγ (FIGS. 25A-25C) or rosiglitazone (FIGS. 25D-25F), and converse regulation of this molecular pathway was observed during normoxia and hypoxia by inhibition of the miR-130/301 family but not miR-130a alone (FIGS. 6A-6C). Notably, we analyzed other downstream targets of STAT3 such as NFATC2 and PIM1 (FIG. 26), known to be important in proliferation (7, 41) as well as other PH-relevant phenotypes (42). Manipulation of either miR-130/301 or PPARγ modulated these target genes, suggesting a broad impact of the miR-130/301-PPARγ-STAT3 axis in control of PASMC-specific phenotypes beyond proliferation alone. Nonetheless, as with miR-424/503 in PAECs, forced miR-204 expression prevented the specific proliferative response of PASMCs to miR-130a (FIG. 6D). Thus, validating the second arm of network predictions in PASMCs, the miR-130/301-PPARγ regulatory axis controls proliferation primarily by repressing STAT3 expression and activity and subordinate miR-204 expression (FIG. 6E).

The miR-130/301 Family Controls PPARγ and Subordinate miRNA Pathways to Promote PH In Vivo:

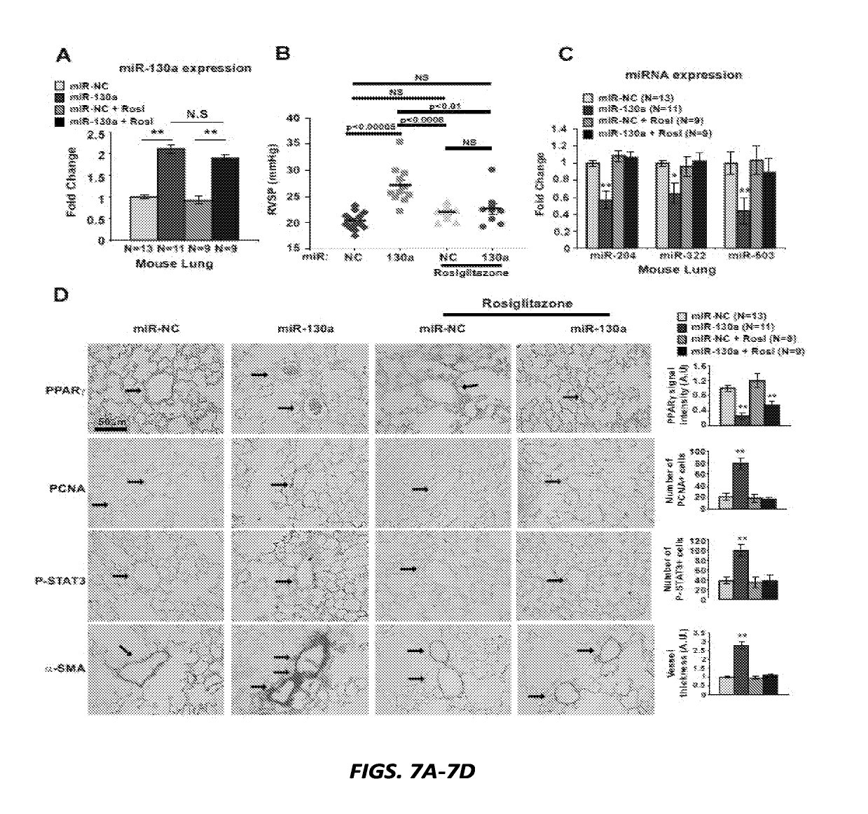

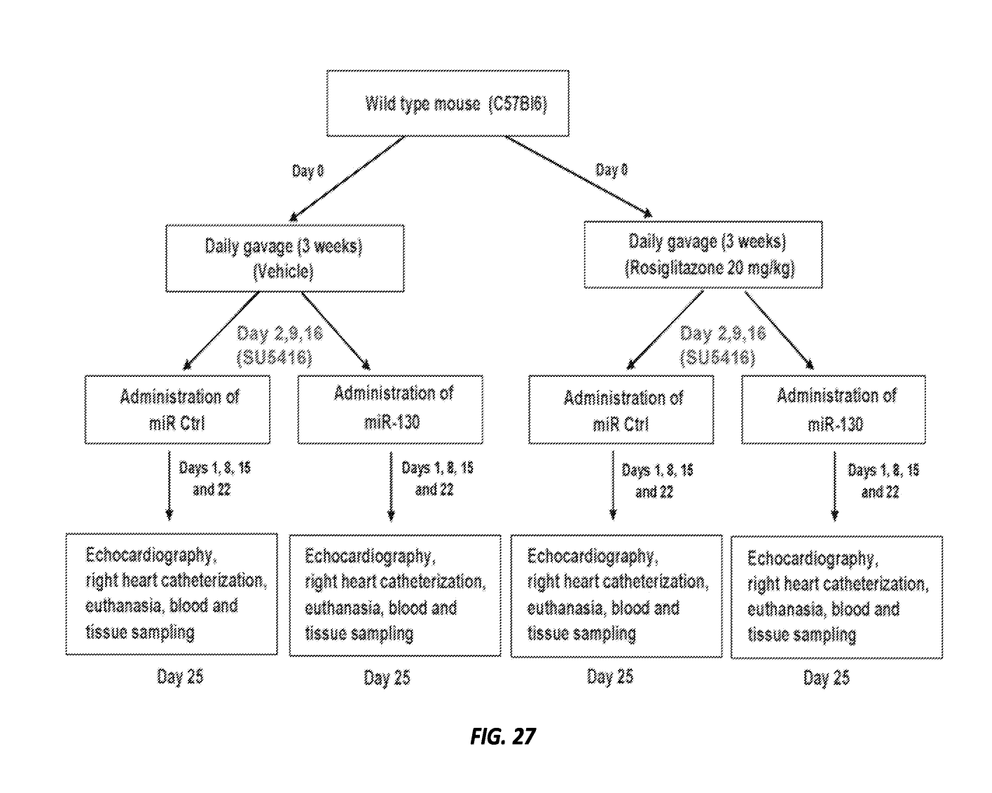

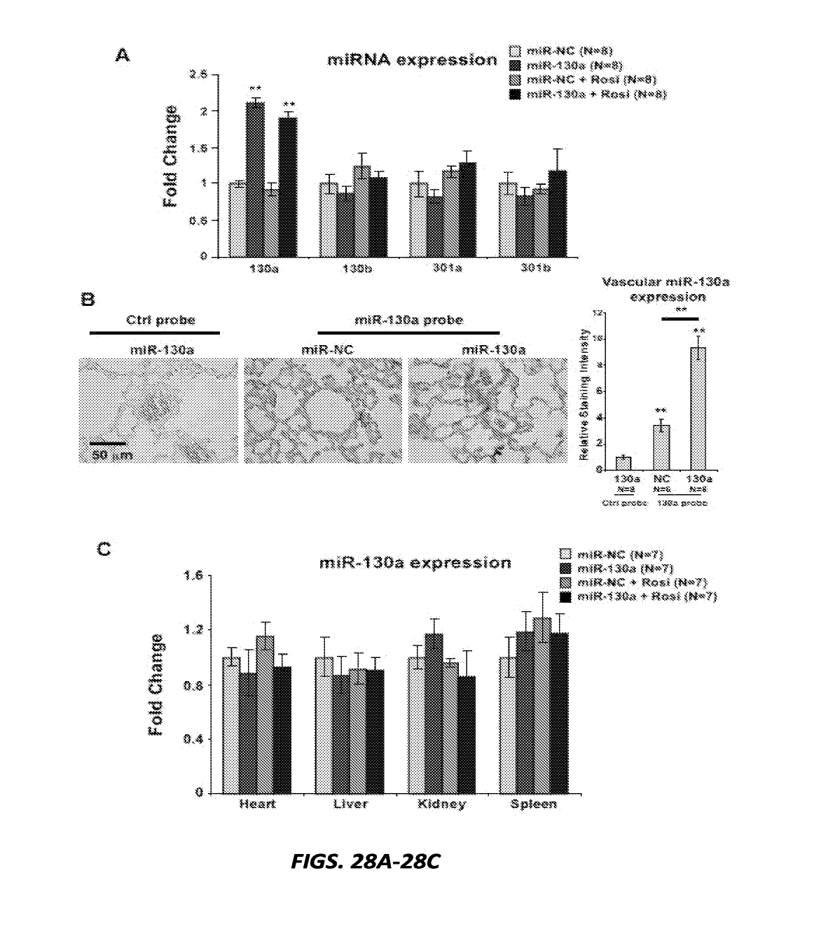

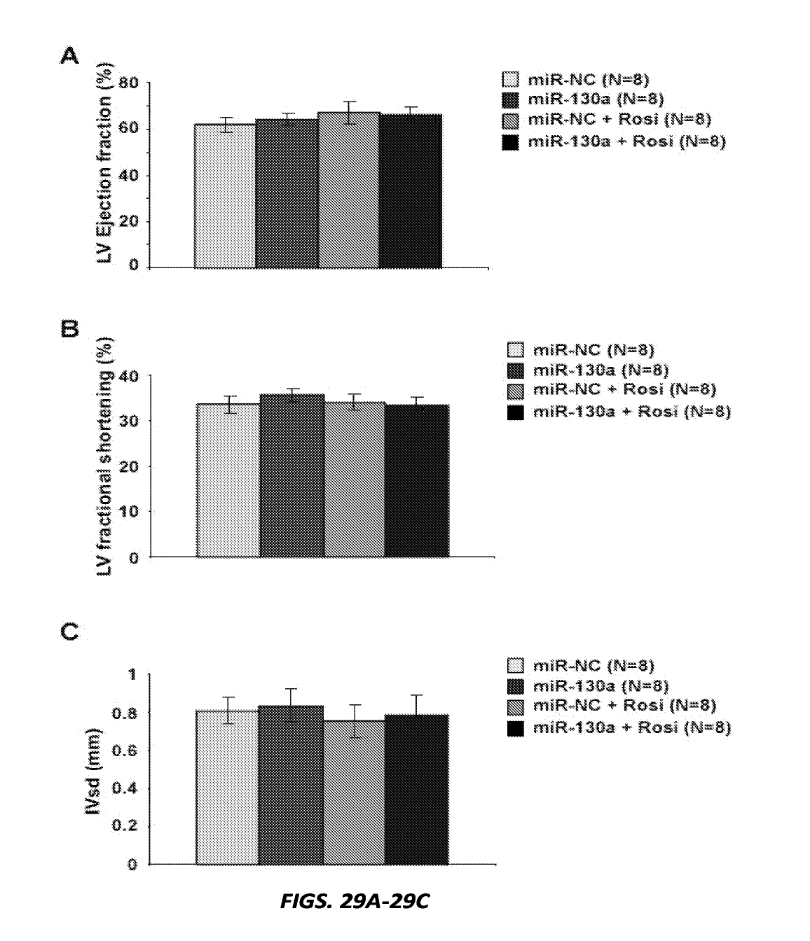

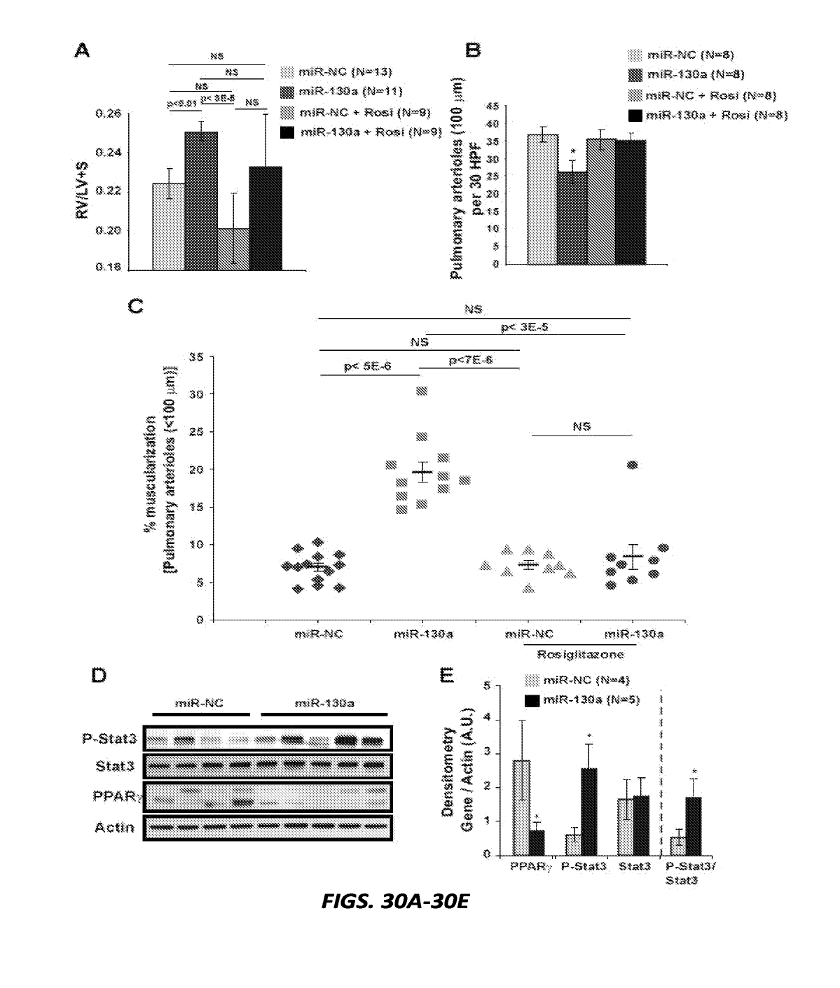

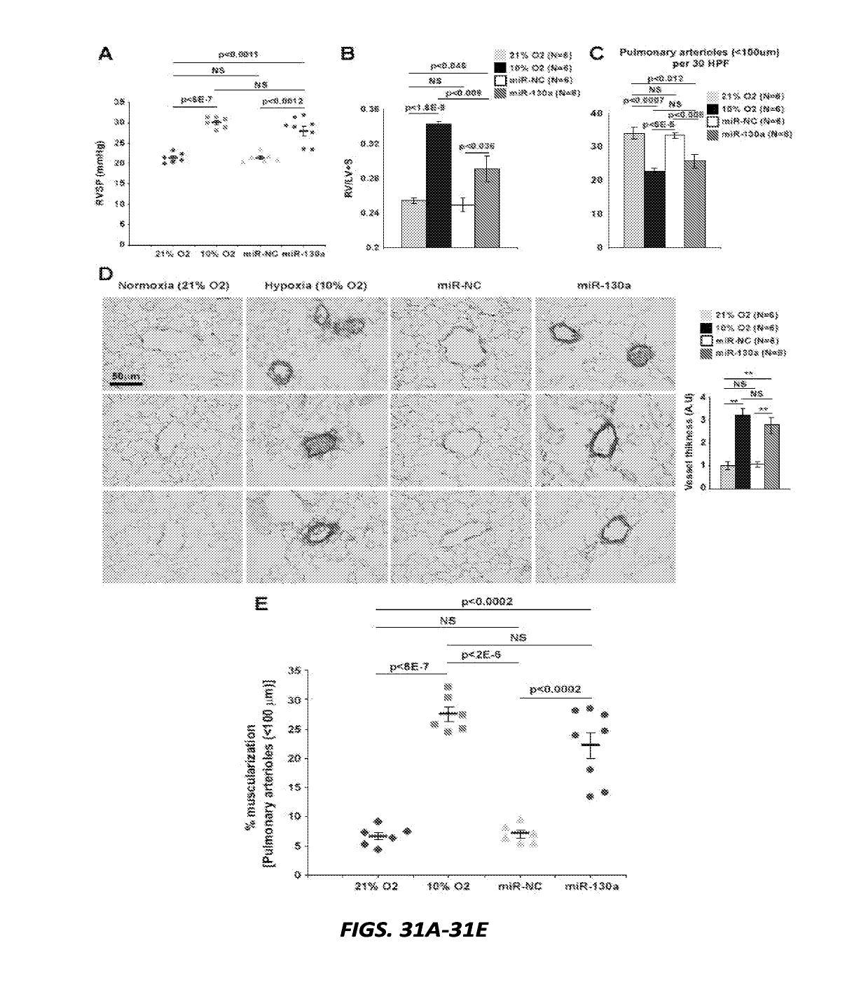

Based on the integrated functions of the miR-130/301 family as delineated above, we wanted to determine whether chronic miR-130/301 induction is necessary and sufficient to promote PH. To maintain consistency in these experiments, a mouse model of PH was employed via treatment with SU5416 and chronic hypoxia (28). First, in the presence of SU5416, chronic pulmonary expression of miR-130a in wildtype mice was studied in place of chronic hypoxia. MiRNA delivery was achieved by 4 serial weekly intrapharyngeal injections of liposomally encapsulated miR-130a oligonucleotide mimics, as adapted from prior protocols (7) (FIG. 27). Such delivery resulted in up-regulation of miR-130a (but not other family members) in whole lung tissue (FIG. 7A, FIG. 28A) and in small pulmonary vessels (FIG. 28B). This delivery also repressed expression of pulmonary vascular PPARγ (as quantified by immunohistochemistry [IHC] methods as previously described (9) [FIG. 7D] and by immunoblot [FIGS. 30D-30E]). Notably, such local delivery confined expression changes predominantly to the lung (FIG. 28C), without alteration in left ventricular function as assessed by echocardiography (FIG. 29). In turn, miR-130a expression up-regulated pulmonary vascular STAT3 phosphorylation (IHC [FIG. 7D] and immunoblot [FIGS. 30D-30E]) and repressed miR-204, miR-322 (the homolog of miR-424 in mice), and miR-503 expression (FIG. 7C), thus leading to vascular proliferation as reflected by PCNA labeling (FIG. 7D). As a result, chronic miR-130a expression increased pulmonary vascular remodeling as demonstrated by increased medial thickness in small (<100 μm diameter) pulmonary arterioles (α-smooth muscle actin stain, FIG. 7D), increased percentage of muscularized arterioles (FIG. 30C) and decreased the number of small pulmonary arterioles (FIG. 30B). In turn, pulmonary vascular expression of miR-130a significantly increased right ventricular remodeling (increased RV/LV+S mass ratio, FIG. 30A) and right ventricular systolic pressure (RVSP), a hemodynamic surrogate for pulmonary arterial pressure (FIG. 7B). Importantly, similar PH manifestations were observed in mice treated with miR-130a without SU5416 (FIG. 31). Notably, miR-130a-dependent gene expression changes as well as most histologic and hemodynamic alterations (FIG. 7 and FIG. 30) were reversed by rosiglitazone, thus demonstrating the necessary role of PPARγ for these pathogenic events in vivo. Thus, miR-130a expression depends, at least in part, upon repression of PPARγ to control a predicted hierarchy of subordinate miRNA pathways in vivo in order to promote pulmonary vascular disease.

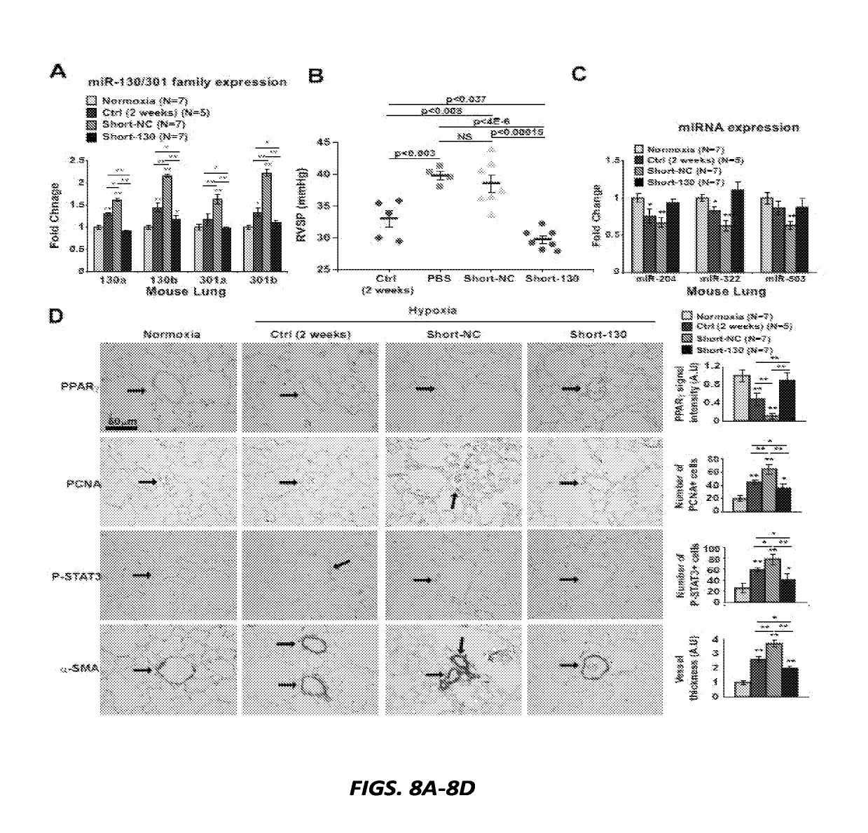

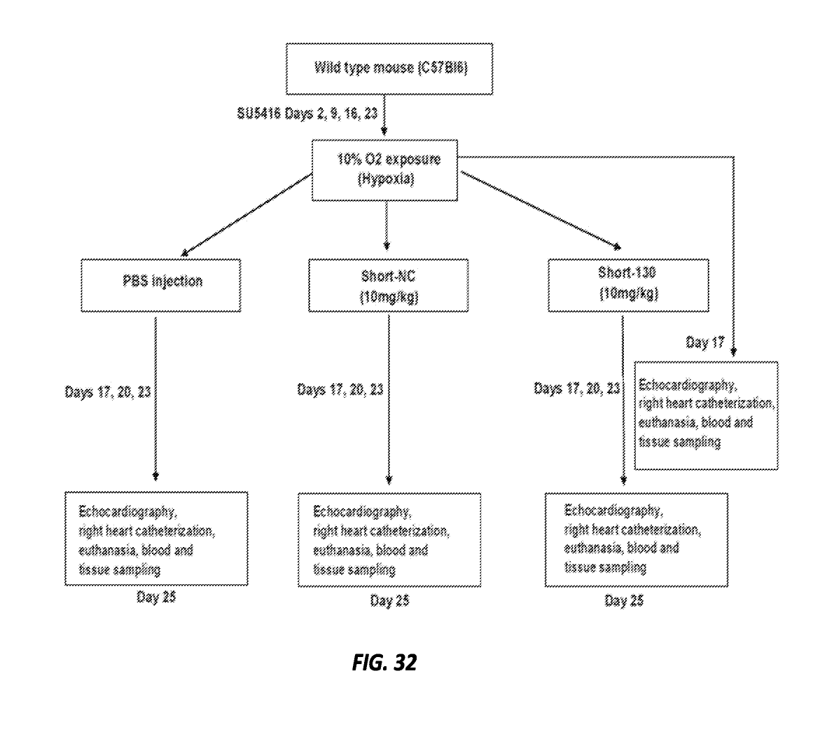

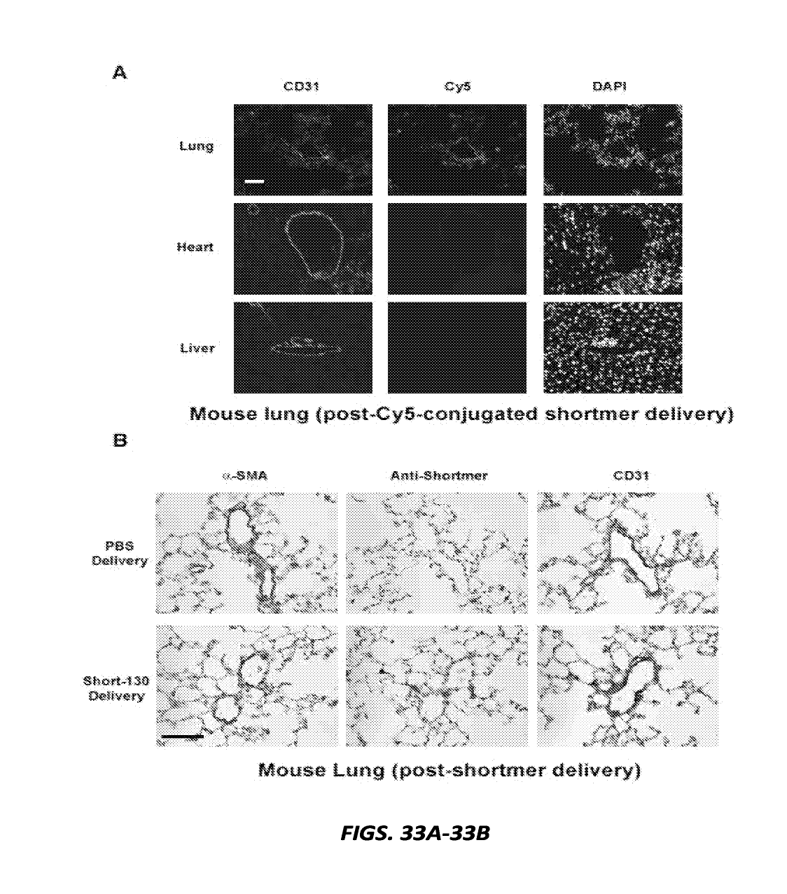

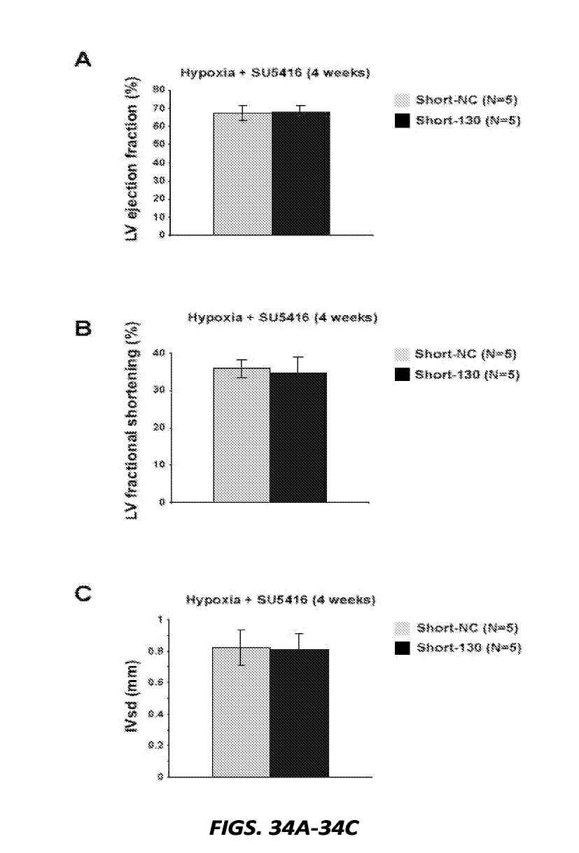

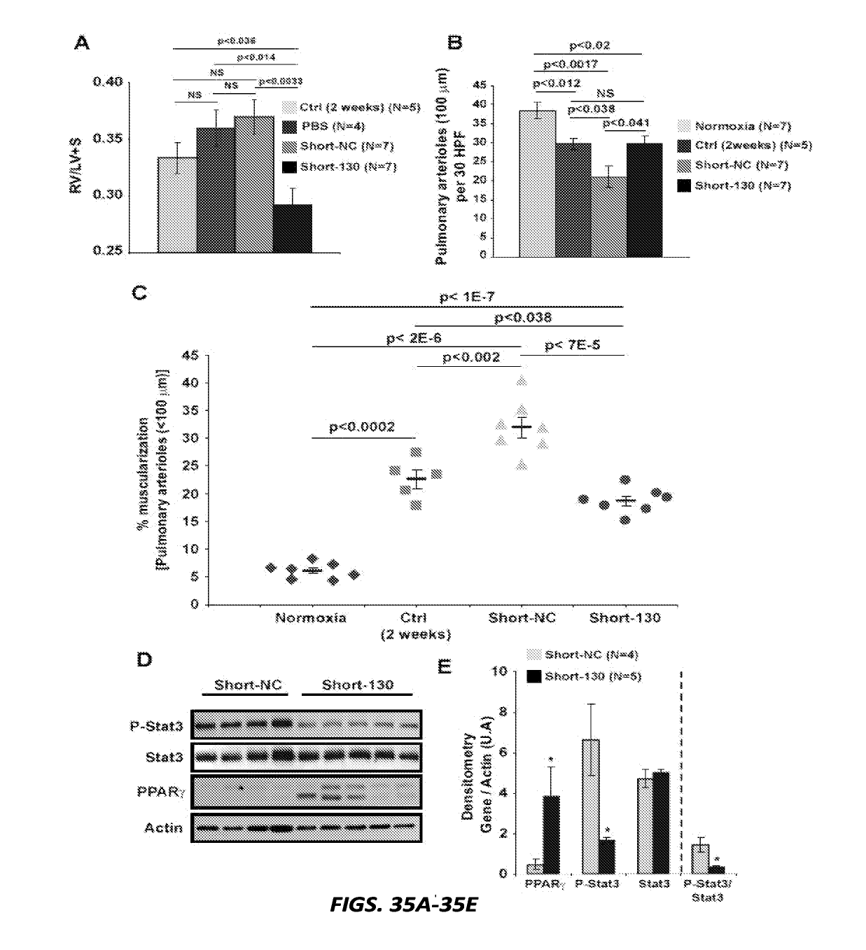

To determine whether miR-130/301 is necessary for manifestation of PH under hypoxic stimuli, inhibition of this miRNA family in the pulmonary vasculature was achieved by pharmacologic means. After two weeks of SU5416 and chronic hypoxia, PH was confirmed in mice by RVSP elevation (FIG. 8B) as well as right ventricular remodeling (FIG. 35A) and pulmonary arteriolar remodeling (FIG. 8D and FIGS. 35B-35C). This treatment was followed by two additional weeks of hypoxia+SU5416 in the setting of 3 serial intrapharyngeal injections of a chemically-modified, short anti-miR complementary to the seed sequence (e.g., “shortmer”) or scrambled control (FIG. 32). Shortmer delivery was confirmed specifically in pulmonary tissue as assessed by repression of the miR-130-301 family members to baseline, non-diseased conditions in whole lung by RT-qPCR (FIG. 8A), in lung vasculature by immunohistochemical staining of the shortmer backbone (FIG. 33B), and in lung vasculature by visualization of a fluorescent Cy5-labeled shortmer (FIG. 33A). Consistent with our mode of intrapharyngeal delivery, such shortmer expression was not seen in other organs beyond the lung (FIG. 33A), including the left ventricle where function that was unaffected with such administration (FIG. 34). In direct correlation with our mechanistic findings defined in cultured PAECs and PASMCs (FIGS. 5-6), during disease provocation, inhibition of the miR-130/301 family (short-130) de-repressed pulmonary vascular PPARγ expression in order to decrease STAT3 phosphorylation (by IHC [FIG. 8D] and immunoblot [FIGS. 35D-35E]), increase expression of its subordinate miRNAs miR-204, miR-424, and miR-503 (FIG. 8C), and decrease vascular proliferation as reflected by PCNA expression (FIG. 8D). Importantly, such molecular events markedly reduced pulmonary vascular remodeling (reflected by medial thickness [FIG. 8D] and percent of muscularized arterioles [FIG. 35C] after IHC stain for α-smooth muscle actin), loss of small pulmonary arterioles (FIG. 35B), right ventricular remodeling (FIG. 35A), and RVSP (FIG. 8B), with a trend toward levels even lower than the starting point of disease (2 week time point). Thus, considering both gain-of-function and loss-of-function experimentation, we conclude that chronic induction of endogenous miR-130/301 family members is necessary and sufficient to promote PH in vivo, and such robust actions depend upon hierarchical control of PPARγ and subordinate miRNAs, as predicted by our network-based analyses.

### Discussion

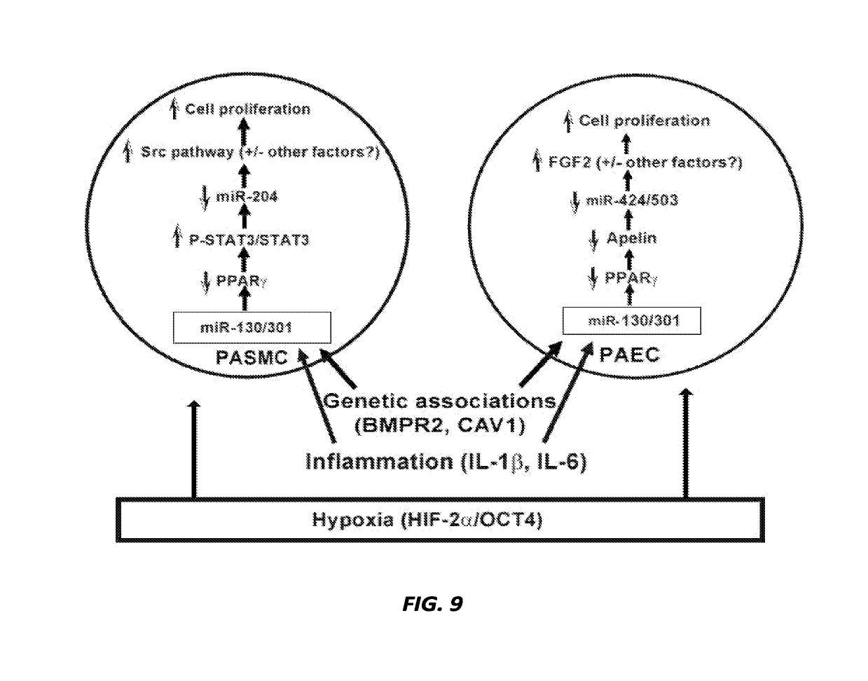

In this study, driven by an advanced analysis of the molecular network architecture specific for PH, we identified unique systems-level regulation of this disease by miR-130/301, orchestrating subordinate miRNA and target gene networks to control cellular proliferation and PH manifestation in vivo. Importantly, the suppression of miR-204 and miR-424/503 by the miR-130/301 family represents the first description to our knowledge of a functional hierarchy of seemingly disparate miRNAs relevant to PH. Furthermore, we found that such broad control extends not only to multiple signaling pathways but also across disparate cell types. Thus, these data (FIG. 9) emphasize that a single set of upstream master miRNAs integrates the control of pulmonary vascular cross-talk and PH-relevant phenotypes via interconnected mechanisms that had previously been missed by conventional discovery strategies. Accordingly, such findings have important implications for our fundamental understanding of the upstream origins of PH, for improvements in diagnostic and therapeutic applications in this disease, and for the increased use of in silico network theory to decipher additional systems-level regulatory mechanisms of miRNAs in complex disease networks.

These findings provide critical validation for the evolving application of network theory to the discovery of the combinatorial origins of PH and other diseases. To date, systems biology approaches have been developed to elucidate the higher-order regulation of human disease gene networks mainly in single cell contexts amenable to high-throughput profiling, such as in cancer (43). However, such profiling has not been possible in diseases such as PH, where diseased tissue is challenging to obtain and pathogenesis spans multiple heterogeneous cell types [as we review in (44)]. Consequently, substantial concern has been raised regarding the ability to discern the key factors—miRNAs or otherwise—controlling PH phenotypes among the exponentially increasing interconnections being mapped among PH-relevant genes, effectors, and cell types. On the other hand, we and others have proposed computational analyses of the overarching architecture of existing integrated molecular disease networks to bypass some of these obstacles [as reviewed by (10)]. However, in vivo validation of these theories had been limited until now. Here, primarily based on statistical analyses of available network architecture, our use of network theory and experimental biology indeed predicts and confirms an expansive miRNA-based disease network in PH and supports the notion that available molecular maps in PH are adequate for such modeling. Mapping of the complete disease interactome would be ideal, but anatomic inaccessibility of pulmonary vascular cell types in vivo and financial infeasibility for extensive measurements limit such data in PH (45). The level of over-determination and redundancy in complex biological networks (10) may be a key factor in allowing us to perceive such higher-order regulation even if some genes or pathways are missing from the current interactome maps. Consequently, future endeavors to map similar miRNA-target networks in PH and other diseases hold great promise for rapidly identifying other master regulators otherwise hidden in the architecture of existing disease networks.

The actions of the miR-130/301 family as a collective rather than reliance on one individual miRNA may underlie their combinatorial robustness of action in PH. The cohesive up-regulation of the miR-130/301 family as a unit despite separate chromosomal locations reflects the evolutionary selection pressure to coordinate their actions. The importance of their simultaneous actions is further emphasized by the intricate yet distinct methods by which these miRNAs are induced under various PH triggers. These include a NF-κB-dependence during inflammatory cytokine stimulation (13), a dependence on BMPR2 (FIG. 10C and FIG. 12C) and CAV1 (FIG. 10), and a unique HIF-2α-POU5F1/OCT4 dependence during hypoxia (FIG. 2). The intriguing dependence of miR-130/301 expression on BMPR2 and CAV1 sets the stage for deeper mechanistic interrogation, perhaps regarding whether endogenous mutations in both of these genes are functionally linked to PAH via this miRNA family. Furthermore, given the previously described association of PH with a gain-of-function mutation of HIF-2α seen in persons with Chuvash polycythemia (46), it is tempting to speculate the importance of the miR-130/301 family in the pathobiology of this genetic predisposition as well. Pathogenic alterations of other unidentified regulatory factors may up-regulate this miRNA family in the context of other PH triggers. Intriguing possibilities may include dehydroepiandrosterone (DHEA) (47) or the DNA damage effector Poly(ADP-ribose) polymerase-1 (PARP-1) (48), both of which influence the STAT3-miR-204 axis in PH. Ultimately, such convergent biology (FIG. 1) could reflect synergism among disparate stimuli to up-regulate miR-130/301 more robustly during PH progression—actions that may be best delineated through further network-based bioinformatics analyses. To coordinate miRNA function, principles of redundancy and synergy may also figure prominently but have been poorly defined in vivo, especially in PH. For the miR-130/301 family that shares the same “seed sequence,” our primary data emphasize the biologic importance of target binding redundancy to allow for the maintenance of miRNA action without relying upon one factor alone. Such results also highlight the importance for strategic inhibition of multiple related miRNAs for maximum therapeutic effect. Finally, future work can be envisioned to interrogate the synergy of action of multiple miR-130/301 family members that bind the same target with varying efficiencies (i.e., typically determined by “non-seed sequences”) and may depend upon coordinated effects on miRNA binding and mRNA structure to maximize target gene repression.

Importantly, while this report focused mainly on proliferative actions of miR-130/301, our experiments validated only a small minority of the network predictions. Thus, the true breadth of influence by the miR-130/301 family in PH may be even more extensive. Specifically, our findings expand upon the importance of PPARγ in PH (19-21, 24, 49), revealing a more complete spectrum of its miRNA-dependent functions in the pulmonary vasculature. The known pleiotropy of PPARγ influencing vasoconstriction, metabolism, and extracellular matrix deposition in the peripheral vasculature and as suggested by our work examining additional PPARγ targets responsive to miR-130/301 (FIG. 20) further suggests that this miRNA family controls even more PH-specific phenotypes than elucidated here. Moreover, our data should prompt future work to determine a more detailed molecular explanation of how the miR-130/301-PPARγ axis controls both context-specific apoptotic signaling (FIG. 18) and proliferation (FIG. 5) in PAECs in order to converge upon promoting PH. Beyond PPARγ, additional miR-130/301 targets may regulate a variety of PH-relevant pathways including TGF/BMP signaling, Rho-kinase activation, vascular smooth muscle contraction, and mitochondrial metabolism, among others (FIG. 1C). Moreover, beyond the miR-130/301 family, both subordinate miRNAs miR-424 and miR-204 themselves were predicted in the top-10 miRNAs by miRNA spanning score as comparably broad regulators of the expanded PH network (Table 2). Taken together, convergent regulation of all of these factors by the miR-130/301 family may offer additional layers of cooperativity and ultimately may allow for both more precise and more robust control over disease manifestation than most previously characterized PH disease genes.

A molecular appreciation of the systems-level actions of the miR-130/301 family could also aid the development of more effective clinical management strategies for PH. From a diagnostic angle, expanding upon prior studies (50), we found that expression of the miR-130/301 family in pulmonary arterial plasma increased with worsening hemodynamic severity of PH (FIG. 4C). Coupled with the related functions of these miRNAs in PH, such results provide a foundation for a larger study aimed at characterizing this miRNA family as a profile of PH biomarkers which could offer more specificity as compared with a single miRNA alone. From a therapeutic angle, the unique application of shortmer technology (34) to pulmonary vascular disease in vivo could impact substantially the rational treatment of PH by inhibiting multiple, but related, miRNAs for greater amelioration of disease. Moreover, a rational combination of pharmacologic PPARγ activation along with repression of this miRNA family could have even synergistic effects on protection and reversal of this disease. Consequently, we envision that further experimental validation of miRNA network architecture may have a dramatic impact on systems pharmacology approaches in PH which otherwise have not yet been pursued in great depth.

In summary, through advanced analysis and validation of disease network architecture, we have defined a higher order of miRNA network regulation in PH by the miR-130/301 family, thus addressing a notable deficiency in reductionist experimentation and carrying broad implications for miRNA-based diagnostics and therapeutics. Consequently, future applications of miRNA network theory should rapidly define additional upstream origins of PH and perhaps other disease conditions that link complex miRNA signaling pathways to final disease manifestations.

### References for Example 1

1. Matkovich S J, Hu Y, Dorn G W. Regulation of cardiac microRNAs by cardiac microRNAs. Circ Res. 2013; 113:62-71.
2. Song S J, Poliseno L, Song M S, et al. MicroRNA-antagonism regulates breast cancer stemness and metastasis via TET-family-dependent chromatin remodeling. Cell. 2013; 154:311-324.
3. Shi H, Xu J, Zhang G, et al. Walking the interactome to identify human miRNA-disease associations through the functional link between miRNA targets and disease genes. BMC Syst Biol. 2013; 7:101.
4. Lai X, Bhattacharya A, Schmitz U, Kunz M, Vera J, Wolkenhauer O. A Systems Biology Approach to Study MicroRNA-Mediated Gene Regulatory Networks. Biomed Res Int. 2013; 2013:703849.
5. Gurtan A M, Sharp P A. The role of miRNAs in regulating gene expression networks. J Mol Biol. 2013; 425:3582-3600.
6. White K, Loscalzo J, Chan S Y. Holding our breath: The emerging and anticipated roles of microRNA in pulmonary hypertension. Pulm Circ. 2012; 2:278-290.
7. Courboulin A, Paulin R, Giguere N J, et al. Role for miR-204 in human pulmonary arterial hypertension. J Exp Med. 2011; 208:535-548.
8. Kim J, Kang Y, Kojima Y, et al. An endothelial apelin-FGF link mediated by miR-424 and miR-503 is disrupted in pulmonary arterial hypertension. Nat Med. 2013; 19:74-82.
9. Parikh V N, Jin R C, Rabello S, et al. MicroRNA-21 Integrates Pathogenic Signaling to Control Pulmonary Hypertension: Results of a Network Bioinformatics Approach. Circulation. 2012; 125:1520-1532.
10. Barabasi A L, Gulbahce N, Loscalzo J. Network medicine: a network-based approach to human disease. Nat Rev Genet. 2011; 12:56-68.
11. Friedman R C, Farh K K, Burge C B, Bartel D P. Most mammalian mRNAs are conserved targets of microRNAs. Genome Res. 2009; 19:92-105.
12. Newman M E. Modularity and community structure in networks. Proc Natl Acad Sci USA. 2006; 103:8577-8582.
13. Lu Z, Li Y, Takwi, A, et al. miR-301a as an NF-kappaB activator in pancreatic cancer cells. EMBO J. 2011; 30:57-67.
14. Kovaleva V, Mora R, Park Y J, et al. miRNA-130a targets ATG2B and DICER1 to inhibit autophagy and trigger killing of chronic lymphocytic leukemia cells. Cancer Res. 2012; 72:1763-1772.
15. Hager M, Pedersen C C, Larsen M T, et al. MicroRNA-130a-mediated down-regulation of Smad4 contributes to reduced sensitivity to TGF-beta1 stimulation in granulocytic precursors. Blood. 2011; 118:6649-6659.
16. Chen Y, Gorski D H. Regulation of angiogenesis through a microRNA (miR-130a) that down-regulates antiangiogenic homeobox genes GAX and HOXA5. Blood. 2008; 111:1217-1226.
17. Wu W H, Hu C P, Chen X P, et al. MicroRNA-130a mediates proliferation of vascular smooth muscle cells in hypertension. Am J Hypertens. 2011; 24:1087-1093.
18. Mujahid S, Nielsen H C, Volpe M V. MiR-221 and miR-130a regulate lung airway and vascular development. PLoS One. 2013; 8:e55911.
19. Hansmann G, de Jesus Perez V A, Alastalo T P, et al. An antiproliferative BMP-2/PPARgamma/apoE axis in human and murine SMCs and its role in pulmonary hypertension. J Clin Invest. 2008; 118:1846-1857.
20. Crossno J T, Garat C V, Reusch J E, et al. Rosiglitazone attenuates hypoxia-induced pulmonary arterial remodeling. Am J Physiol Lung Cell Mol Physiol. 2007; 292:L885-897.
21. Nisbet R E, Bland J M, Kleinhenz D J, et al. Rosiglitazone attenuates chronic hypoxia-induced pulmonary hypertension in a mouse model. Am J Respir Cell Mol Biol. 2010; 42:482-490.
22. Liu Y, Tian X Y, Mao G, et al. Peroxisome proliferator-activated receptor-gamma ameliorates pulmonary arterial hypertension by inhibiting 5-hydroxytryptamine 2B receptor. Hypertension. 2012; 60:1471-1478.
23. Rabinovitch M. PPARgamma and the pathobiology of pulmonary arterial hypertension. Adv Exp Med Biol. 2010; 661:447-458.
24. Alastalo T P, Li M, Perez Vde J, et al. Disruption of PPARgamma/beta-catenin-mediated regulation of apelin impairs BMP-induced mouse and human pulmonary arterial E C survival. J Clin Invest. 2011; 121:3735-3746.
25. Soubrier F, Chung W K, Machado R, et al. Genetics and genomics of pulmonary arterial hypertension. J Am Coll Cardiol. 2013; 62:D13-21.
26. Covello K L, Kehler J, Yu H, et al. HIF-2 alpha regulates Oct-4: effects of hypoxia on stem cell function, embryonic development, and tumor growth. Genes Dev. 2006; 20:557-570.
27. Firth A L, Yao W, Remillard C V, Ogawa A, Yuan J X. Upregulation of Oct-4 isoforms in pulmonary artery smooth muscle cells from patients with pulmonary arterial hypertension. Am J Physiol Lung Cell Mol Physiol. 2010; 298:L548-557.
28. Ciuclan L, Bonneau O, Hussey M, et al. A Novel Murine Model of Severe Pulmonary Arterial Hypertension. Am J Respir Crit Care Med. 2011; 184(10):1171-1182.
29. Steiner M K, Syrkina O L, Kolliputi N, Mark E J, Hales C A, Waxman A B. Interleukin-6 overexpression induces pulmonary hypertension. Circ Res. 2009; 104:236-244, 228p following 244.
30. Graham B B, Mentink-Kane M M, El-Haddad H, et al. Schistosomiasis-induced experimental pulmonary hypertension: role of interleukin-13 signaling. Am J Pathol. 2010; 177:1549-1561.
31. West J, Fagan K, Steudel W, et al. Pulmonary hypertension in transgenic mice expressing a dominant-negative BMPRII gene in smooth muscle. Circ Res. 2004; 94:1109-1114.
32. Reddy V, Meyrick B, Wong J, et al. In utero placement of aortopulmonary shunts. A model of postnatal pulmonary hypertension with increased pulmonary blood flow in lambs. Circulation. 1995; 92:606-613.
33. Lee E K, Lee M J, Abdelmohsen K, et al. miR-130 suppresses adipogenesis by inhibiting peroxisome proliferator-activated receptor gamma expression. Mol Cell Biol. 2011; 31:626-638.
34. Obad S, dos Santos C O, Petri A, et al. Silencing of microRNA families by seed-targeting tiny LNAs. Nat Genet. 2011; 43:371-378.
35. Hansmann G, Wagner R, Schellong S, et al. Pulmonary arterial hypertension is linked to insulin resistance and reversed by peroxisome proliferator-activated receptor-gamma activation. Circulation. 2007; 115:1275-1284.
36. Hansmann G, Zamanian R T. PPARgamma activation: a potential treatment for pulmonary hypertension. Sci Transl Med. 2009; 1:12ps14.
37. Li, M, Li, Z, Sun, X, et al. Heme oxygenase-1/p21WAF1 mediates peroxisome proliferator-activated receptor-gamma signaling inhibition of proliferation of rat pulmonary artery smooth muscle cells. FEBS J. 2010; 277:1543-1550.
38. Teichert-Kuliszewska K, Kutryk M, Kuliszewski M, et al. Bone morphogenetic protein receptor-2 signaling promotes pulmonary arterial endothelial cell survival: implications for loss-of-function mutations in the pathogenesis of pulmonary hypertension. Circ Res. 2006; 98:209-217.
39. Sakao S, Taraseviciene-Stewart L, Lee J, Wood K, Cool C, Voelkel N. Initial apoptosis is followed by increased proliferation of apoptosis-resistant endothelial cells. FASEB Journal. 2005; 19:1178-1180.
40. Wang L H, Yang X Y, Zhang X, et al. Transcriptional inactivation of STAT3 by PPARgamma suppresses IL-6-responsive multiple myeloma cells. Immunity. 2004; 20:205-218.
41. Paulin R, Courboulin A, Meloche J, et al. Signal transducers and activators of transcription-3/piml axis plays a critical role in the pathogenesis of human pulmonary arterial hypertension. Circulation. 2011; 123:1205-1215.
42. Malenfant S, Neyron A S, Paulin R, et al. Signal transduction in the development of pulmonary arterial hypertension. Pulm Circ. 2013; 3:278-293.
43. Lee M J, Ye A S, Gardino, A K, et al. Sequential application of anticancer drugs enhances cell death by rewiring apoptotic signaling networks. Cell. 2012; 149:780-794.
44. Chan S Y, Loscalzo J. The emerging paradigm of network medicine in the study of human disease. Circ Res. 2012; 111:359-374.
45. Chan S Y, Loscalzo J. Pathogenic mechanisms of pulmonary arterial hypertension. J Mol Cell Cardiol. 2008; 44:14-30.
46. Hickey M M, Richardson T, Wang T, et al. The von Hippel-Lindau Chuvash mutation promotes pulmonary hypertension and fibrosis in mice. J Clin Invest. 2010; 120:827-839.
47. Paulin R, Meloche J, Jacob M H, Bisserier M, Courboulin A, Bonnet S. Dehydroepiandrosterone inhibits the Src/STAT3 constitutive activation in pulmonary arterial hypertension. Am J Physiol Heart Circ Physiol. 2011; 301:H1798-1809
48. Meloche J, Pflieger A, Vaillancourt M, et al. Role for DNA Damage Signaling in Pulmonary Arterial Hypertension. Circulation. 2013; 129(7):786-797.
49. Kang B Y, Kleinhenz J M, Murphy T C, Hart C M. The PPARgamma ligand rosiglitazone attenuates hypoxia-induced endothelin signaling in vitro and in vivo. Am J Physiol Lung Cell Mol Physiol. 2011; 301:L881-891.
50. Wei C, Henderson H, Spradley C, et al. Circulating miRNAs as potential marker for pulmonary hypertension. PLoS One. 2013; 8:e64396.

### Example 2

### Methods

Network Design.

A complete set of methods to construct the initial curated and expanded PH networks and to perform spectral partition-based clustering of those networks are included in Supplemental Data. Supplemental Data also contain detailed methods for analysis of network architecture, including calculations for the miRNA spanning score, target spanning score, gene spanning score, and shared miRNA influence score.

Oligonucleotides and Transfection.

Pre-miRNA oligonucleotides (pre-miR-130a, pre-miR-204, pre-miR-424, and pre-miR-503 and negative control pre-miR-NC1 and premiR-NC2, Life Technologies), anti-miRNAs oligonucleotides (anti-miR-130a and anti-miR-NC, ThermoScientific), tiny LNA oligonucleotides (tiny-130: 5′-ATTGCACT-3′ (SEQ ID NO: 3), and tiny-NC 5′-TCATACTA-3′ (SEQ ID NO: 51), Exiqon), siRNAs (PPARγ and scrambled control, Santa Cruz Biotechnology; HIF-1α, HIF-2α, and scrambled control, Life Technologies) were commercially purchased. PAECs and PASMCs were plated and transfected 24 h later at 70-80% confluence using pre-miRNA (5 nM), anti-miRNA (20 nM), tiny-LNA (20 nM) or siRNA (25 nM) with Lipofectamine 2000 reagent (Life Technologies), according to the manufacturers' instructions.

Forced Expression of miR-130a in Mouse Lung.

Eight-week-old mice (C57Bl6) were injected with SU5416 weekly (20 mg/kg/dose; Sigma-Aldrich), accompanied by 4 intrapharyngeal injections (once by week) of 1 nmol of miR-control (pre-miR-NC) or miR-130a (pre-miR-130a) mixed in 100 uL PBS solution containing 5% Lipofectamine 2000 (Life Technologies). Three days after the last injection, right heart catheterization was performed as previously described (9), followed by harvest of lung tissue for RNA extraction or paraffin embedding, as described above. Rosiglitazone (Cayman Chemicals) was dissolved in 0.25% carboxymethyl cellulose medium viscosity aqueous solution. This rosiglitazone solution versus vehicle control was delivered daily (20 mg/kg/day) by oral gavage to mice for 3 weeks.

Inhibition of the miR-130/301 Family in Mouse Lung.

Eight-week-old mice (C57Bl6) were injected with SU5416 SU5416 (20 mg/kg/dose; Sigma-Aldrich), followed by exposure to normobaric hypoxia (10% O2; OxyCycler chamber, Biospherix Ltd, Redfield, N.Y.) for 2 weeks. After two weeks and confirmation of PH development in five mice (right heart catheterization), mice were further treated with 3 intrapharyngeal injections (every 4 days) of control or miR-130/301 shortmer oligonucleotides, designed as fully modified antisense oligonucleotides complementary to the seed sequence of the miR-130/301 miRNA family (10 mg/kg/dose; Regulus). Specifically, the control and miR-130/301 shortmer oligonucleotides were non-toxic, lipid-permeable, high-affinity oligonucleotides. The miR-130/301 shortmer carried a sequence complementary to the active site of the miR-130/301 miRNA family, containing a phosphorothioate backbone and modifications (fluoro, methoxyethyl, and bicyclic sugar) at the sugar 2′ position. Three day after the last injection, right heart catheterization was performed followed by harvest of lung tissue for RNA extraction or paraffin embedding, as described above.

Statistics.

Cell culture experiments were performed at least three times and at least in triplicate for each replicate. The number of animals in each group was calculated to measure at least a 20% difference between the means of experimental and control groups with a power of 80% and standard deviation of 10%. The number of unique patient samples for this study was determined primarily by clinical availability. RT-qPCR on human plasma, in situ expression/histologic analyses of both mouse and human tissue, and pulmonary vascular hemodynamics in mice were performed in a blinded fashion. Numerical quantifications for in vitro experiments using cultured cells or in situ quantifications of transcript/miRNA expression represent mean±standard deviation (SD). Numerical quantifications for physiologic experiments using mice or human reagents represent mean±standard error of the mean (SEM). Immunoblot images are representative of experiments that have been repeated at least three times. Micrographs are representative of experiments in each relevant cohort of mice. Paired samples were compared by Student's t test (two-tailed). Comparison of multiple samples was performed by ANOVA followed by Student Newman-Keuls post hoc testing. Correlation analyses were performed by Pearson correlation coefficient calculation, as previously described (8). A P value less than 0.05 was considered significant.

Network Design.

To construct the initial PH network, we used a previously described curated set of PH-relevant genes (1). These genes were selected based on their co-occurrence with the search term “pulmonary hypertension” in the Medline (PubMed) database. As we described (1), interactions between network genes were annotated according to a master list of protein-protein interactions (2-6), protein-DNA interactions (7), kinase-substrate interactions (8, 9), and metabolic interactions (10) drawn from several consolidated databases. These interactions together formed a “consolidated interactome,” consisting of 11,643 nodes and 113,765 edges. These edges represented a wide range of functional relationships including transcriptional, translational, and protein level associations. Notably, the consolidated interactome used for these analyses was further expanded to include information detailed in the Reactome Functional Interaction Network (11), which reports coverage of roughly 50% of the human proteome. When mapped onto this expanded consolidated interactome, the curated PH-relevant genes formed a network consisting of 115 nodes and 398 edges, with a largest connected component (LCC) of 105. We refer to the network formed from this set as the “curated PH network.”

In order to capture novel disease genes that may not have been reported in the literature, we also constructed an expanded network which included genes that could be shown to interact extensively with curated PH genes but which were not present in a literature search at the time of network construction. We refer to this larger network as the “expanded PH network” (listed in Table 1). In order to generate the expanded PH network, we first defined a set of “PH interactors.” An interactor was defined as any node in the consolidated interactome that (a) shared at least one edge with at least one gene in the curated PH network, and (b) was not itself a member of the curated PH network. We then ranked these interactors by their shortest-path betweenness centrality score, considering only shortest paths between curated PH network genes. Broadly speaking, the shortest-path betweennness centrality (12) of a node v in a network G represents the amount of network connectivity that would be lost were v to be removed from G.

C_{B}(v)=Σ_{t≠t}σₛₜ(v)/σₛₜ Eq 1:

Where v is a PH interactor, s and t are curated PH network genes, σₛₜ represents the number of shortest paths from node s to node t in the consolidated interactome, and σₛₜ(v) represents the number of such paths that must pass through node v. This process was performed iteratively. For each iteration, the highest scoring node was incorporated into the PH network, and the betweenness centrality scores of the remaining interactors were recomputed to reflect this change. When the normalized betweenness centrality score of the highest scoring interactor fell below a fixed threshold, no further nodes were added to the network. The resulting network contained 249 nodes and 2,274 edges, with the LCC encompassing all 249 nodes.

MiRNA Target Prediction:

MiRNA target prediction was performed using the well-validated algorithm, TargetScan 6.2 (Conserved) algorithm (13). The TargetScan algorithm detects mRNA transcripts with conserved complimentarily to the “seed sequence” (nucleotides 2-7) of a given miRNA. Because of this, miRNAs that share a seed sequence are grouped together as a family and regarded as a single unit by the algorithm. For this reason, we do not distinguish between miRNAs belonging to the same family in any of our statistical analyses.

Network Clustering:

Clustering was performed using the Reactome FI analysis tool in the Cytoscape 2.8.1 environment (11, 14). This tool uses a spectral partition-based clustering algorithm which partitions networks in order to maximize the density of connectivity within a group (or “cluster”) of genes. This is measured by the modularity (Q) of the graph. Modularity here is defined as the fraction of edges within a given cluster, minus the fraction that would be expected by chance if the edges in the network were distributed randomly (15). This is computed as follows:

Q=Σᵢ^{c}(eᵢ−aᵢ²) Eq 2a:

eᵢ=ΣⱼAᵢⱼ/2mδ(cᵢ,cⱼ) Eq 2b:

aᵢ=kᵢ/2m Eq 2c:

Where Aᵢⱼ represents the adjacency matrix of graph G, kᵢ represents the degree of node i in G, m represents the total number of edges in G, c represents the number of clusters into which G is to be partitioned, eᵢ represents the fraction of edges for which both vertices are contained in cluster i, and aᵢ represents the fraction of edges for which at least one vertex is contained in cluster i. In order to cluster the network optimally, the algorithm aims to maximize modularity. When modularity is high, it indicates that the graph has been partitioned in such a way that the number of edges that fall within clusters is large, and the number that fall between clusters is comparatively small. Thus, this algorithm ensures that clusters are chosen such that the boundaries of each cluster cross as few edges as possible.

### Analyses of Network Architecture

Identification of miRNAs that Broadly Regulate the Expanded PH Network (miRNA Spanning Score):

To identify miRNAs that might serve as broad regulators of multiple nodes and pathways embedded throughout the PH network, we designed an in silico network-based strategy to rank miRNAs according to two criteria: (a) the reliability of their predicted interaction with PH-relevant genes and (b) the breadth of their impact on the PH network as a whole. To do so, we defined a metric that we refer to as the “miRNA spanning score.” This score is computed as follows:

SPAN(miR)=(CLUST_{miR}/CLUST_{MAX})+(log(p_{miR})/log(p_{MIN})) Eq 3:

CLUST_{miR} represents the number of clusters containing at least one target of the miRNA miR and p_{miR} represents the hypergeometric p-value for the number of PH-relevant targets in the miR target pool. In order to ensure that each component of the score (i.e. number of targeted clusters and hypergeometric p-value) carries roughly equal weight, the components are normalized by their theoretical maximum value. Thus, CLUST_{MAX} represents the total number of clusters in the expanded PH network, and p_{MIN} represents the theoretical minimum p-value, defined as the reciprocal of the number of simulations used to estimate the hypergeometric distribution (100,000 simulations in this case). In this way, all components of the score are computed out of 1, and the overall miRNA spanning score has a maximum possible value of 2. The first term of the score, CLUST_{miR}/CLUST_{MAX}, gives a measure of the miRNA's spread within the network. A miRNA whose targets are distributed throughout all clusters in the network is more likely to influence a wider range of disease-relevant processes, compared with a miRNA whose targets are concentrated within a single network cluster. The second term, log(p_{miR})/log(p_{MIN})), gives a measure of prediction accuracy. Because miRNAs tend to target multiple genes within a given pathway (16), a tendency that is is particularly evident in PH (1), it is reasonable to expect a PH-relevant miRNA to target a large number of PH network genes, relative to the size of its target pool. By relying on the p-value, rather than the absolute number of PH-relevant targets, we avoid rewarding miRNAs for simply having a large number of targets in the TargetScan database. Together, these two measures provide a means of assessing the reliability of the prediction that a given miRNA is involved in disease, as well as a means of estimating the fraction of the network that will be affected, directly or indirectly, when the expression of this miRNA is altered. MiRNAs that perform well, according to this metric, are thus good candidates for exerting a powerful and widespread effect on the PH network.

Identification of downstream pathways controlled by the miR-130/301 family in PH: In order to predict which miR-130/301 family targets and related downstream pathways may be the most influential in the progression of PH, we performed several different analyses. These strategies were chosen to interrogate, as broadly as possible, the various methods by which a single miRNA could exert global control over a related gene network(s) specific to PH.

(1) Target Spanning Score:

To discern which of the miR-130/301 targets likely exerts the broadest influence over the PH network, we developed a spanning score for network nodes, modified from the miRNA spanning score described above (eq. 3). For gene targets, this was computed as follows:

SPAN(v)=(CLUSTᵥ/CLUST_{MAX})+C_{B}(v)+(log(p_{miR})/log(p_{MIN})) Eq 4:

Where C_{B} represents the shortest-path betweenness centrality score of node v, normalized according to the theoretical maximum betweenness centrality score, defined as ½(N−1)(N−2) for an undirected network of N nodes (i.e. the total number of non-redundant node pairs), pmiR represents the hypergeometric p-value of the number of targets in v's first degree neighborhood that are also targeted by miR-130/301, and CLUSTv, CLUST_{MAX}, and p_{MIN} are defined as described above. This score provides a measure both gene v's influence over the network as a whole, as well as miR-130/301's influence over gene v and its immediate neighbors. Genes that perform well, according to this metric, can be said to be both highly influential in the disease network as a whole and highly sensitive to the influence of the miR-130/301 family. Thus, they are ideal “bridges” between the activity of the miR-130/301 family and the rest of the expanded PH network.

(2) Gene Spanning Score:

To capture non-target genes that may nonetheless participate in the miR-130/301 regulatory pathway, we also ranked all genes in the expanded PH network according to their gene spanning score. For non-targets, this was computed as follows:

SPAN(v)=(CLUSTv/CLUST_{MAX})+C_{B}(v)+C_{D}(v) Eq 5:

Where C_{D} represents the degree of node v in the network, normalized according to the theoretical maximum degree, defined as N−1 for a network of N nodes (i.e., the total number of nodes not equal to v), and CLUSTᵥ, CLUST_{MAX}, and C_{B}(v) are all computed as discussed above. Genes that perform well, according to this metric, can be predicted to exert robust control over the PH network. While they need not be targeted directly by the miR-130/301 family, their wide sphere of influence makes them statistically more likely to participate in those functional pathways that participate in the miR-130/301-mediated regulation of disease.

(3) Shared miRNA Influence Score:

To capture miRNAs that may be associated with the miR-130/301-dependent regulation of PH, we ranked each conserved miRNA in the TargetScan database according to the hypergeometric p-value of the overlap of its pool of targets and first-degree target interactors with those genes also directly targeted by miR-130/301. MiRNA that perform well according to this metric can be said to have a high degree of functional overlap with the miR-130/301 family.

Plasmids:

3′UTR sequence from PPARγ was cloned in the pSI-CHECK-2 vector (Promega) downstream of the Renilla luciferase using XhoI and NotI restrictions sites. Mutagenesis of the putative miR-130-family binding sites was performed using the QuickChange multisite-directed mutagenesis kit (Stratagene/Agilent) according to the manufacturer's protocol. Coding sequence of PPARγ (BC006811) was amplified by polymerase chain reaction (PCR) using high-fidelity polymerase Phusion (Thermo Fisher Scientific) from an MGC cDNA clone (clone ID: 3447380) and inserted in the pCDH-CMV-MCS-EF1-copGFP (System Biosciences) using NheI and NotI restrictions sites. To achieve constitutive expression of HIF-1α, a mutant HIF-1α expression construct that carries alanine substitutions at proline sites targeted by prolyl-hydroxylases (HA-HIF1alpha P402A/P564ApBabe-puro, a generous gift from W. G. Kaelin (17)) was used. The lentiviral parent vector expressing GFP was used as a control. Stable expression of these constructs in PAECs or PASMCs was achieved by lentiviral transduction. All cloned plasmids were confirmed by DNA sequencing.

Cell Culture and Reagents:

Primary human pulmonary arterial endothelial cells (PAECs) were purchased and propagated in EGM-2 cell culture media (Lonza). Experiments were performed at passages 3 to 6. Primary human pulmonary arterial smooth muscle cells (PASMCs) were purchased and propagated in SmGM-2 cell culture media (Lonza). Experiments were performed at passages 3 to 9. HEK293T cells (American Type Culture Collection) were used and cultivated in DMEM containing 10% fetal bovine serum (FBS). Cells were cultured at 37° C. in a humidified 5% CO₂ atmosphere. A modular hypoxia chamber at 0.2% oxygen, 94.8% nitrogen and 5% carbon dioxide was used during 24 h for hypoxic conditions. Cultured cells were exposed to recombinant human IL-1α at 10 ng·mL-1 (Peprotech). Recombinant human IL-6 (Peprotech) was used at 100 ng·mL-1. Rosiglitazone (Sigma) was used at 10 μM.

Human Plasma Sampling: To collect blood from subjects from the main pulmonary artery, clinically indicated right heart catheterization procedures were performed by standard protocol via a right internal jugular approach under fluoroscopic guidance. The catheter was positioned into the main pulmonary artery, as confirmed by fluoroscopy and hemodynamic waveforms. Blood was then drawn from the distal catheter port and collected in standard vacutainer tubes with K+-EDTA anticoagulant. Plasma was extracted after standard centrifugation of blood, followed by storage at −80° C.

Animals:

All animal treatments and analyses were conducted in a controlled and non-blinded manner.

VHL flox/flox; Cre-ER mice (C57Bl6 background, >10 backcrosses) were a generous gift from W. G. Kaelin (Dana Farber Cancer Institute, Boston). Conditional inactivation of VHL was performed by treating 3-week-old VHL flox/flox; Cre-ER mice with 2 mg of tamoxifen (Sigma Aldrich) by intraperitoneal injection every other day for 2 doses as we previously described (18), followed by tissue harvest at 10-13 weeks of age. Tissue from tamoxifen-treated, gender-(male), and age-matched VHL flox/flox mice (without the Cre-ER transgene) was used as wildtype comparison (referred to as VHL WT).

IL-6 transgenic mice (C57Bl6 background) have been described previously (19).

BMPR2X transgenic mice: Briefly, we used the Rosa26-rtTA2×TetO7-Bmpr2R899X FVB/N mice previously described (20). Control mice expressed the Rosa26-rtTA2 gene only, but were otherwise identically treated. Expression of transgene occurred only after initiation of doxycycline. Adult mice (12-16 weeks of age) were fed doxycycline in normal (low fat) chow for 6 weeks. Echocardiogram and RVSP were performed at 6 weeks as previously described (21) and the animals sacrificed for harvesting of tissues. Lungs were snap frozen in liquid nitrogen immediately upon sacrifice.

Schistosoma mansoni-infected mice: Mice were exposed to Schistosoma mansoni ova to cause experimental PH, using a published technique (22, 23). In brief, eight week old C57Bl6/J mice were used for the study. Experimentally exposed mice were intraperitoneally sensitized on day one and then intravenously challenged on day 14 with S. mansoni eggs, at a dose of 175 eggs/gram body weight at each timpoint. S. mansoni eggs were obtained from homogenized and purified livers of Swiss-Webster mice infected with S. mansoni cercariae, provided by the Biomedical Research Institute (Rockville, Md.). On day 21, right ventricular catheterization was performed followed by tissue and blood collection. The mice were anesthetized with ketamine/xylazine and ventilated through a transtracheal catheter. The abdominal and thoracic cavities were opened, and a 1Fr pressure-volume catheter (Millar PVR-1035, Millar Instruments) was placed through the right ventricle apex to transduce the pressure. Blood samples (400 μl) were drawn after catheterization using into a syringe containing 100 μl 0.5M EDTA at pH 8.0. Blood samples were centrifuged at 2000 g for 20 minutes at 4° C. to separate plasma. The remaining blood was flushed out of the lungs with PBS, the right bronchus was sutured, and 1% agarose was instilled into the left lung through the transtracheal catheter. The left lung was removed, formalin-fixed, and processed for paraffin embedding for histology. The right lungs were removed and snap frozen. The right ventricle free wall was dissected off of the heart and weighed relative to the septum and left ventricle to measure hypertrophy (the Fulton index).

Wildtype mice exposed to chronic hypoxia+/−SU5416: Eight-week-old littermate mice (C57Bl6) were serially injected intraperitoneally with SU5416 (20 mg/kg/weekly dose; Sigma-Aldrich), followed by exposure to normobaric hypoxia (10% O₂; OxyCycler chamber, Biospherix Ltd, Redfield, N.Y.) or normoxia (21% O₂) for 3 weeks. Right heart catheterization was then performed followed by harvest of lung tissue for RNA extraction or paraffin embedding. Separately, mice were treated in chronic hypoxia as compared with normoxia without SU5416 administration.

Monocrotaline-treated rats: As previously described (1), male Sprague-Dawley rats (10-14 week old) were injected with 60 mg/kg monocrotaline at time 0; at 0-4 weeks post-exposure, right heart catheterization was performed followed by harvest of lung tissue for RNA extraction or paraffin embedding, as described below (section: Tissue harvest).

Surgical placement of PA-aortic shunts in juvenile lambs: As previously described (24), pregnant mixed-breed Western ewes (137-141 d gestation, term=145 d) were anesthetized. Fetal exposure was obtained through the horn of the uterus; a left lateral thoracotomy was performed on the fetal lamb. With the use of side biting vascular clamps, an 8.0-mm vascular graft was anastomosed between the ascending aorta and main pulmonary artery of the fetal lambs. Four weeks after spontaneous delivery shunt and control (provided by twin pregnancy or age-matched) lambs were anesthetized and catheters were placed to measure hemodynamics including left pulmonary blood flow. After baseline hemodynamics were obtained, peripheral lung was obtained for analysis. At the end of the protocol, all lambs were euthanized with a lethal injection of sodium pentobarbital followed by bilateral thoracotomy as described in the National Institutes of Health Guidelines for the Care and Use of Laboratory Animals.

In Silico Determination of Transcription Factors Predicted to Bind the Promoters of the Different Members of miR-130/301 Family:

We consulted ChIPBase (25), a large database of chromatin immunoprecipitation with next generation DNA sequencing (ChIP-Seq) data, to determine which transcription factors might be modulating miR-130/301 family expression under hypoxia. For each member of the miR-130/301 family, we composed a list of all transcription factors thought to target miRNA promoters based on ChIP-Seq experiments performed in human tissues and cell lines. We then cross-referenced these lists to produce a final set of twelve transcription factors thought to target the promoters of at least four out of the five miR-130/301 family members. Finally, we further narrowed this list by consulting the Transcription Factor Encyclopedia for a curated list of known HIF-2α transcriptional targets (26).

Lentivirus Production:

HEK293T cells were transfected using Lipofectamine 2000 (Life Technologies) with lentiviral plasmids along with packaging plasmids (pPACK, System Biosciences), according to the manufacturer's instructions. Virus was harvested, sterile filtered (0.45 um), and utilized for subsequent infection of PAECs or PASMCs (24-48 hour incubation) for gene transduction.

miRNA Target Validation by Luciferase Assay:

Adapted from our previously published protocol (18), HEK293T cells were plated in 96-well plates and transfected with 200 ng of pSICHECK-2 constructs and 5 nM of pre-miRNAs using Lipofectamine 2000 (Life Technologies). The medium was replaced 8 hours after transfection with fresh medium containing 10% FCS, L-glutamine. 48 hours after transfection, firefly and Renilla Luciferase activities were measured using the Dual-Glo™ Luciferase assay (Promega).

BrdU Proliferation Assay:

Exponentially growing cells were transfected, collected at the indicated times and counted. For proliferation assays, 5-bromo-2-uridine (BrdU) was added to the cell culture medium for 1 hour, and BrdU incorporated into the DNA was revealed using a detection kit (BrdU Cell Proliferation Assay Kit #6813, Cell Signaling) according to the manufacturer's instructions.

Caspase 3/7 Assay:

Caspase 3,7 activity was quantified using the Caspase-Glo 3/7 Assay (Promega), according to manufacturer's instructions. Briefly, cells were plated in triplicate in 96-well plates and transfected with the appropriate oligonucleotide. Forty-eight hours after transfection, cells were cultured in the presence or absence of serum for twenty-four hours. Cells were then incubated for 1 h with the caspase substrates, and luminescence was quantified.

Plasma RNA Extraction:

To remove any residual cellular contents, thawed plasma samples were further centrifuged (13,000 rpm×10 minutes), and plasma supernatant was aliquoted into 100 ul volumes for storage at −80° C. or further analysis. Specifically, to a standard volume of each plasma supernatant (100 μl), 2 picomoles were added of a chemically synthesized miRNA duplex mimic of microRNA-422b (miR-422b) (Life Technologies). Given minimal expression and minimal changes of expression of endogenous miR-422b (27), equivalent levels of exogenously added miR-422b were used for quantitative normalization of miRNA plasma levels, as previously described (27). Plasma miRNA extraction was performed using a microRNA extraction kit (MicroRNA Extraction System, Benevbio).

Messenger RNA and miRNA Extraction:

Cells were homogenized in 2 ml of QiaZol reagent (Qiagen). Total RNAs including small RNAs were extracted using the miRNeasy kit (Qiagen) according to the manufacturer's instructions. Total RNA concentration was determined using a ND-1000 micro-spectrophotometer (NanoDrop Technologies).

Quantitative RT-PCR of Mature miRNAs:

Mature miRNA expression was evaluated using TaqMan MicroRNA Assays (Life Technologies/Applied Biosystems) and the Applied Biosystems 7900HT Fast Real Time PCR device (Life Technologies/Applied Biosystems). Expression levels were normalized to RNU48 or snoR55 for human or mouse experiments, respectively, and calculated using the comparative Ct method (2^{-ΔΔCt}) as we previously described (1).

Quantitative RT-PCR of Messenger RNAs:

Messenger RNAs were reverse transcribed using the Multiscript RT kit (Life Technologies) to generate cDNA. cDNA was amplified via fluorescently labeled Taqman primer sets using an Applied Biosystems 7900HT Fast Real Time PCR device, as we previously described (1). Fold-change of RNA species was calculated using the formula (2^{-ΔΔCt}), normalized to actin expression.

Immunoblotting and Antibodies:

Cells were lysed in RIPA buffer (Santa Cruz Biotechnology) and the protein concentration determined using a Bradford assay (Biorad). Protein lysate (40 mg) were resolved by SDS-PAGE and transferred onto a PVDF membrane (Biorad). Membranes were blocked in 5% non-fat milk in TN buffer (50 mM Tris-HCl pH 7.4, 150 mM NaCl) and incubated in the presence of the primary and then secondary antibodies. After washing in TN buffer containing 0.1% Tween, immunoreactive bands were visualized with the ECL system (Amersham Biosciences). Primary polyclonal or monoclonal antibodies to APLN (sc 33469), EDN1 (sc 21625), FGF2 (sc 79), NOS3 (sc 654), PPARγ (sc 7273) and VEGFA (sc 152) were obtained from Santa Cruz Biotechnology. Primary antibodies for STAT3 (#9139) and P-STAT3 (Tyr-705; #9145) were purchased from Cell Signaling. Primary antibodies against actin (ab 3280), α-smooth-muscle-actin (ab 32575), CD31 (ab28364), and PCNA (ab 29) were obtained from Abcam. Appropriate secondary antibodies (anti-rabbit and anti-mouse) coupled to HRP were used (Santa Cruz Biotechnology).

Tissue Harvest:

After physiological measurements, blood was extracted by cardiac puncture for future analyses (hematocrit, plasma extraction). By direct right ventricular puncture, the pulmonary vessels were gently flushed with 1 cc of saline to remove the majority of blood cells, prior to harvesting cardiopulmonary tissue. The heart was removed, followed by dissection and weighing of the right ventricle (RV) and of the left ventricle+septum (LV+S). Organs were then harvested for histological preparation or flash frozen in liquid N2 for subsequent homogenization and extraction of RNA and/or protein. To further process lung tissue specifically, prior to excision, lungs were flushed with PBS at constant low pressure (˜10 mmHg) via right ventricular cannulation, followed by tracheal inflation of the left lung with 10% neutral-buffered formalin (Sigma-Aldrich) at a pressure of −20 cm H2O. After excision and 16 hours of fixation in 10% neutral-buffered formalin at 25° C., lung tissues were paraffin-embedded via an ethanol-xylene dehydration series, before being sliced into 5 μm sections (Hypercenter XP System and Embedding Center, Shandon).

In Situ Hybridization:

The protocol for in situ hybridization for miRNA detection was based on a prior report (28). Specifically, 5 μm sagittal lung sections were probed using a 3′ fluorescein isothiocyanate (FITC) labeled miRCURY LNA hsa-miR-130a detection probe (Exiqon). The miRCURY LNA scramble-miR probe was used as negative control. Following re-hydration (Sigma) lungs were formaldehyde-fixed (4% formaldehyde, Sigma) before inactivation of endogenous enzymes by acetylation buffer [873 uL of triethanolamine (Sigma) and 375 uL acetic anhydride (Fisher) in 75 ml distilled water]. Probe annealing (25 nM LNA probe) was performed in hybridization buffer (Sigma, H7782) for 16 hours at RNA-Tm-22° C. (62° C.). Following serial washes with 2×SSC, 1×SSC and 0.5×SSC (Sigma) at 62° C., immunolabeling was performed with an anti-FITC biotin-conjugated antibody for overnight at 4° C. (1:400; Sigma-Aldrich). For detection, development was achieved by adding streptavidin-biotinylated alkaline phosphatase complex (Vector Labs) followed by Nitro blue tetrazolium chloride/5-Bromo-4-chloro-3-indolyl phosphate substrate solution (NBT/BCIP, Roche), and positive staining was evident by a blue color. MiR-130a expression was quantified in the vascular wall of 15-20 resistance pulmonary arteries (<100 μm external diameter in rodents and <200 um external diameter in humans) using ImageJ software (NIH).

Immunohistochemistry of Human and Mouse Lung:

Lung sections (5 μm) were deparaffinized and high temperature antigen retrieval was performed followed by blocking in TBS/BSA 5%, 10% goat serum and exposure to primary antibody and biotinylated secondary antibody (Vectastain ABC kit, Vector Labs). In most cases, color development was achieved by adding streptavidin-biotinylated alkaline phosphatase complex (Vector Labs) followed by Vector Red alkaline phosphatase substrate solution (Vector Labs). Levamisole was added to block endogenous alkaline phosphatase activity (Vector Labs). Pictures were obtained using an Olympus Bx51 microscope (40× objective). Small pulmonary vessels (<100 μm diameter) present in a given tissue section (>10 vessels per section) that were not associated with bronchial airways were selected for analysis (N>5 animals per group). Intensity of staining was quantified using ImageJ software (NIH). Degree of pulmonary arteriolar muscularization was assessed in paraffin-embedded lung sections stained for α-SMA by calculation of the proportion of fully and partially muscularized peripheral (<100 μm diameter) pulmonary arterioles to total peripheral pulmonary arterioles, as previously described (29). Medial thickness was also measured in α-SMA stained vessels (<100 μm diameter) using ImageJ software (NIH) and expressed as arbitrary units. All measurements were performed blinded to condition.

Immunohistochemistry to detect shortmer delivery was performed commercially (Wax It Histology Services, Inc.). Specifically, formalin fixed paraffin embedded mouse lung sections were deparaffinised in xylene & rehydrated in graded alcohols. Sections were retrieved in Proteinase K, pH8.0, treated with hydrogen peroxide in methanol for blocking endogenous peroxidase activity, and blocked with protein block to reduce background staining. Sections were then incubated with the primary anti-shortmer antibody E5746-B3A (Regulus Therapeutics) at 1:1000 (0.73 μg/ml) or corresponding negative isotype control Rabbit IgG overnight at 4° C. The following day, the sections were incubated with the HRP Labelled Polymer. Positive staining was visualized with DAB chromagen. Sections were counterstained in Mayer's haematoxylin, blued in lithium carbonate, dehydrated in graded alcohols, cleared in xylene, and mounted with permount.

Pulmonary Arterial Density Quantification:

For quantitative assessment of pulmonary arterial density, a previously published study protocols were adapted here (30, 31), lungs were paraffin embedded, sectioned, and stained for CD31 in order to identify vascular endothelium. The number of CD31+pulmonary vessels (<100 μm diameter) was counted in 30 high-power fields (400×) per mouse lung. Fields containing large airways or major pulmonary arteries were avoided to maintain consistency of counts between sections.

Fluorescent Microscopy of Mouse Lung:

To process lung tissue specifically for fluorescence microscopy, prior to excision, lungs were flushed with PBS at constant low pressure (˜10 mmHg) via right ventricular cannulation, followed by tracheal inflation of the left lung in optimal cutting temperature medium (OCT) (Tissue-Tek, Sakura) at a pressure of ˜20 cm H2O, followed by cryopreservation in ethanol/dry ice. Tissue was then further embedded in OCT, frozen solid in cryomolds, sectioned on a Microm HM 550 at 10 μm, and stored at −80° C. Cryosections were then air dried for 10 min at room temperature and rehydrated in 1×PBS for 15 min at room temperature. Sections were washed in 1×PBS, blocked in 1×PBS/BSA3% supplemented with 10% heat-inactivated goat serum for 1 h at room temperature and then probed with appropriate primary antibody overnight at 4° C. After incubation, slides were washed 3 times with 1×PBS, blocked for 1 h at room temperature and probed with appropriate secondary antibodies in dark room, 1 h at room temperature. After washing, slides were mounted in mounting medium containing DAPI (Vectashield, Vector Labs) and sealed with nail polish.

### References for Example 2

1. Parikh V N, Jin R C, Rabello S, et al. MicroRNA-21 Integrates Pathogenic Signaling to Control Pulmonary Hypertension: Results of a Network Bioinformatics Approach.

Circulation. 2012; 125:1520-1532.
2. Rual J F, Venkatesan K, Hao T, et al. Towards a proteome-scale map of the human protein-protein interaction network. Nature. 2005; 437:1173-1178.
3. Stelzl U, Worm U, Lalowski M, et al. A human protein-protein interaction network: a resource for annotating the proteome. Cell. 2005; 122:957-968.
4. Venkatesan K, Rual J F, Vazquez A, et al. An empirical framework for binary interactome mapping. Nat Methods. 2009; 6:83-90.
5. Aranda B, Achuthan P, Alam-Faruque Y, et al. The IntAct molecular interaction database in 2010. Nucleic Acids Res. 2010; 38:D525-531.
6. Ceol A, Chatr Aryamontri A, Licata L, et al. MINT, the molecular interaction database:
2009 update. Nucleic Acids Res. 2009; 38:D532-539.
7. Wingender E, Chen X, Fricke E, et al. The TRANSFAC system on gene expression regulation. Nucleic Acids Res. 2001; 29:281-283.
8. Diella F, Gould C M, Chica C, Via A, Gibson T J. Phospho. ELM: a database of phosphorylation sites—update 2008. Nucleic Acids Res. 2008; 36:D240-244.
9. Hornbeck P V, Chabra I, Kornhauser J M, Skrzypek E, Zhang B. PhosphoSite: A bioinformatics resource dedicated to physiological protein phosphorylation. Proteomics. 2004; 4:1551-1561.
10. Lee D S, Park J, Kay K A, Christakis N A, Oltvai Z N, Barabasi A L. The implications of human metabolic network topology for disease comorbidity. Proc Natl Acad Sci USA. 2008; 105:9880-9885.
11. Wu G, Feng X, Stein L. A human functional protein interaction network and its application to cancer data analysis. Genome Biol. 2010; 11:R53.
12. Brandes U. On variants of shortest-path betweenness centrality and their generic computation. Social Networks. 2008; 30:2.
13. Friedman R C, Farh K K, Burge C B, Bartel D P. Most mammalian mRNAs are conserved targets of microRNAs. Genome Res. 2009; 19:92-105.
14. Shannon P, Markiel A, Ozier O, et al. Cytoscape: a software environment for integrated models of biomolecular interaction networks. Genome Res. 2003; 13:2498-2504.
15. Newman M E. Modularity and community structure in networks. Proc Natl Acad Sci USA. 2006; 103:8577-8582.
16. Shirdel E A, Xie W, Mak T W, Jurisica I. NAViGaTing the micronome—using multiple microRNA prediction databases to identify signalling pathway-associated microRNAs. PLoS One. 2011; 6:e17429.
17. Ivan M, Kondo K, Yang H, et al. HlFalpha targeted for VHL-mediated destruction by proline hydroxylation: implications for O2 sensing. Science. 2001; 292:464-468.
18. Chan S Y, Zhang Y Y, Hemann C, Mahoney C E, Zweier J L, Loscalzo J. MicroRNA-210 controls mitochondrial metabolism during hypoxia by repressing the iron-sulfur cluster assembly proteins ISCU1/2. Cell Metab. 2009; 10:273-284.
19. Steiner M K, Syrkina O L, Kolliputi N, Mark E J, Hales C A, Waxman A B. Interleukin-6 overexpression induces pulmonary hypertension. Circ Res. 2009; 104:236-244, 228p following 244.
20. West J, Niswender K D, Johnson J A, et al. A potential role for insulin resistance in experimental pulmonary hypertension. Eur Respir J. 2013; 41:861-871.
21. West J, Harral J, Lane K, et al. Mice expressing BMPR2R899× transgene in smooth muscle develop pulmonary vascular lesions. Am J Physiol Lung Cell Mol Physiol. 2008; 295:L744-755.
22. Graham B B, Chabon J, Kumar R, et al. Protective role of IL-6 in vascular remodeling in Schistosoma pulmonary hypertension. Am J Respir Cell Mol Biol. 2013; 49:951-959.
23. Graham B B, Chabon J, Gebreab L, et al. Transforming growth factor-beta signaling promotes pulmonary hypertension caused by Schistosoma mansoni. Circulation. 2013; 128:1354-1364.
24. Reddy V, Meyrick B, Wong J, et al. In utero placement of aortopulmonary shunts. A model of postnatal pulmonary hypertension with increased pulmonary blood flow in lambs. Circulation. 1995; 92:606-613.
25. Yang J H, Li J H, Jiang S, Zhou H, Qu L H. ChIPBase: a database for decoding the transcriptional regulation of long non-coding RNA and microRNA genes from ChIP-Seq data. Nucleic Acids Res. 2013; 41:D177-187.
26. Yusuf D, Butland S L, Swanson M I, et al. The transcription factor encyclopedia. Genome Biol. 2012; 13:R24.
27. Baggish A L, Hale A, Weiner R B, et al. Dynamic Regulation of Circulating MicroRNA during Acute Exhaustive Exercise and Sustained Aerobic Exercise Training J Physiol. 2011; 589:3983-3994.
28. Pena J T, Sohn-Lee C, Rouhanifard S H, et al. miRNA in situ hybridization in formaldehyde and EDC-fixed tissues. Nat Methods. 2009; 6:139-141.
29. Hansmann G, Wagner R, Schellong S, et al. Pulmonary arterial hypertension is linked to insulin resistance and reversed by peroxisome proliferator-activated receptor-gamma activation. Circulation. 2007; 115:1275-1284.
30. Thebaud B, Ladha F, Michelakis E D, et al. Vascular endothelial growth factor gene therapy increases survival, promotes lung angiogenesis, and prevents alveolar damage in hyperoxia-induced lung injury: evidence that angiogenesis participates in alveolarization. Circulation. 2005; 112:2477-2486.
31. Xia Y, Yang X R, Fu Z, et al. Classical transient receptor potential 1 and 6 contribute to hypoxic pulmonary hypertension through differential regulation of pulmonary vascular functions. Hypertension. 2014; 63:173-180.
32. Obad S, dos Santos C O, Petri A, et al. Silencing of microRNA families by seed-targeting tiny LNAs. Nat Genet. 2011; 43:371-378.

### Example 3: The miR-130/301-PPARγ Regulatory Axis Promotes Vasoconstriction in Pulmonary Hypertension

Pulmonary hypertension (PH) is a complex disorder, spanning several heterogeneous cell types throughout the vasculature. As the number and complexity of known pathways known to be involved in PH has expanded, an understanding of global disease gene architecture has become a more pressing need. Previously, we demonstrated that the miR-130/301 microRNA family, acting through its target peroxisome proliferator-activated receptor gamma (PPARγ), serves as a broad regulator of PH by modulating two pro-proliferative pathways across multiple cell types. Here, we employed targeted network theory to identify additional pathogenic pathways regulated by miR-130/301, including those involving vasomotor tone. Based on these predictions, we demonstrated that the miR-130/301-PPARγ axis regulates a panel of vasoactive factors in vivo and in vitro that communicate between diseased PAECs and PASMCs. Of these, we found that endothelin-1, a paracrine factor that modulates vasoconstriction, serves as an integral point of communication between the miR-130/301-PPARγ axis in PAECs and STAT3-mediated vasoconstriction in PASMCs. These findings support the notion that miRNA-based actions previously missed by conventional approaches can be discerned from available molecular information embedded in the PH disease gene network. In doing so, they clarify the expanded role of the miR-130/301 family in the global regulation of PH and further emphasize the importance of molecular crosstalk among the diverse cellular populations involved in PH.

We constructed a network of PH-relevant miR-130/301 family targets and their closest interactors in order to further investigate the integrative actions of this miRNA family. We catalogued the primary functions of this gene network in the context of the pulmonary vasculature, and guided by these predictions, we demonstrated an additional role for the miR-130/301-PPARγ axis in PH as a regulator of intercellular crosstalk and vasoconstriction, as mediated through up-regulation of circulating EDN1.

### Results

The miR-130/301 Family Targets a Wide Range of PH-Relevant Functional Pathways in Silico:

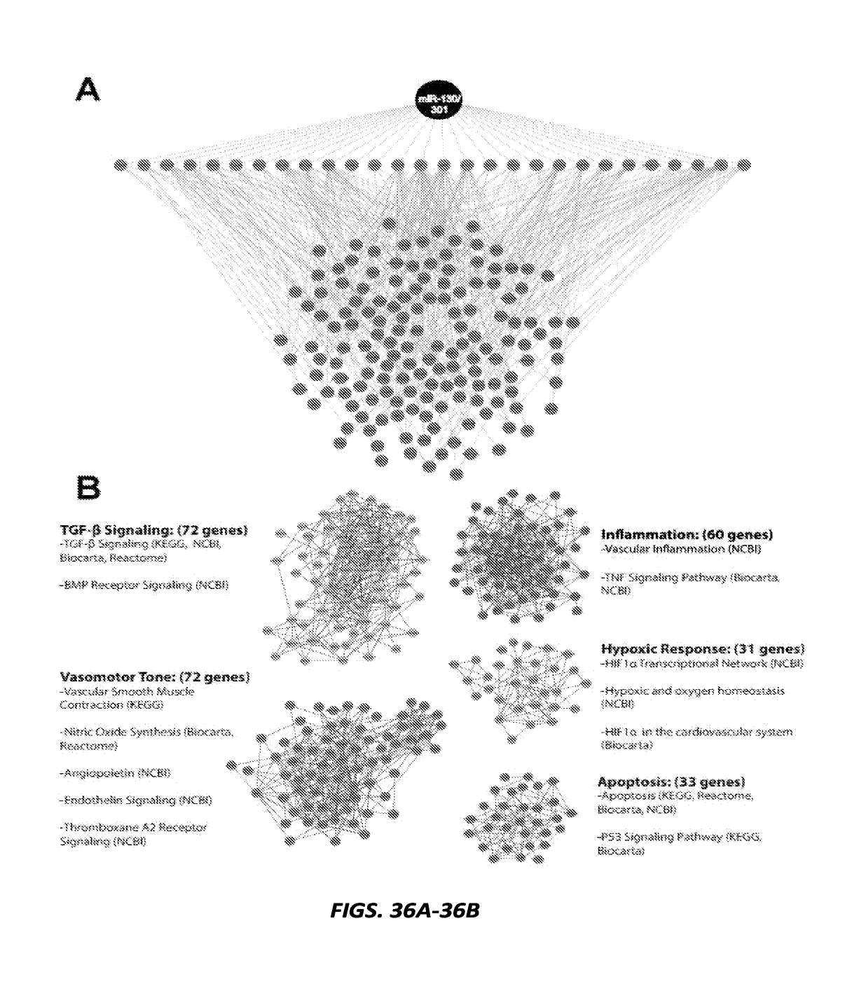

Previously, we reported that miR-130/301 family members are coordinately up-regulated by both hypoxia and inflammatory cytokine stimulation in the diseased pulmonary vasculature (Bertero et al., “Systems-level regulation of microRNA networks by miR-130/301 promotes pulmonary hypertension,” submitted manuscript). As result, PPARγ expression is suppressed, thus allowing for regulation of both endothelial and smooth muscle proliferation. Beyond proliferation, however, additional functions of this pathway are suggested by the broad cohort of predicted PH-relevant miR-130/301 family targets (5), as well as the known pleiotropy of PPARγ in vascular biology as a whole (4). In order to investigate this farther, we returned to our in silico model of the PH functional network. As previously described (Bertero et al., “Systems-level regulation of microRNA networks by miR-130/301 promotes pulmonary hypertension,” submitted manuscript), this network was built using a combination of literature-based curation and network expansion in silico, resulting in a set of 249 genes with known and predicted relevance to PH pathogenesis. Network edges are based on functional molecular associations curated from a variety of human gene and molecular interaction databases (15). We first compared our PH network genes against the TargetScan 6.2 miRNA target prediction algorithm in order to generate a comprehensive set of PH-relevant miR-130/301 family targets. These targets and their first-degree interactors cover more than 70% of the PH network (177 genes), emphasizing the global role that the miR-130/301 family plays in the regulation of this disease (FIG. 36A).

To determine the broad landscape of PH-relevant functions predicted to be controlled by this miRNA family, we next cross-referenced this pool of PH-relevant miR-130/301 family targets and interactors with several curated pathway databases including the Kyoto Encyclopedia of Genes and Genomes (KEGG), Reactome, Biocarta, and the NCI Pathway Interaction Database (NCI PID) (16-19). We found that this miR-130/301 family subnetwork encompasses several broad functional modules (with overlap)—the largest of which are detailed in FIG. 36B (see also Table 7) and include vascular inflammation, vasomotor tone, and control of the hypoxic response. While each targeted module may represent a valid component of the miR-130/301-mediated response, we focused here on the subset of miR-130/301 family targets and interactors that participate in vasoconstriction and control of vasomotor tone, given their inherent importance in control of vascular cell cross-talk. Along with the TGF-β signaling pathway, this module accounts for the largest contingent of the miR-130/301 target subnetwork, and contains several factors, such as STAT3, that we previously established as downstream targets of the miR-130/301-PPARγ axis in the context of PH (Bertero et al., “Systems-level regulation of microRNA networks by miR-130/301 promotes pulmonary hypertension,” submitted manuscript).

TABLE 7
Functions of miR-130/301 Family Targets and First Degree Interactors

| Pathway | Members in miR-130/301 Target Module | Databases |
|---|---|---|
| TGF-β | TGFB1 TGFB2 YWHAQ SMAD9 SMAD4 | KEGG (16) |
| Signaling | SMAD5 FOXG1 SMAD7 SMAD1 SMAD2 | Biocarta (17) |
| | SMAD3 CTNNB1 BMPR1A BMPR1B EGR1 | Reactome (18) |
| | PTPN1 CAV1 DCN EIF3I MAP3K8 CEBPB | NCI PID (19) |
| | CALM1 FOS JUN GDF5 SERPINE1 MAPK3 | |
| | MAPK1 PRKG1 PML PRKACA MAPK8 | |
| | SMAD6 TGFBR2 TGFBR1 ACVRL1 SHC1 | |
| | ENG PRKCA SP1 GRB2 FYN SRC FOXO1 | |
| | PPARG PARD3 SKIL RBX1 HSPA8 RHOA | |
| | EP300 ZEB1 CSNK1A1 BMP7 BMP6 BMP4 | |
| | TP53 BMP2 PPP1CA TGFBR3 ESR1 ROCK1 | |
| | ROCK2 GSK3B PRKCZ BMPR2 RAC1 BCL2 | |
| | YWHAZ PAK1 AR BTBD2 | |
| TNF | TNFSF11 PDGFA FYN NFKB2 NFKBIE AKT1 | Biocarta (17) |
| Signaling | IKBKE CAV1 YWHAZ CEBPB MAP3K8 | NCI PID (19) |
| Pathway | STAT1 CALM1 RAN YWHAQ JUN GNB2L1 | |
| | MAPK3 MAPK1 PRKG1 EGR1 PIK3R1 PML | |
| | NFKB1 RELA GTF2I FOS SRC LIMK1 RAC1 | |
| | PRKCA CHUK PPARG PRKCZ PTPN1 EP300 | |
| | MAPK8 TP53 CSNK1A1 ESR1 PTPN11 GSK3B | |
| | PRKACA HSP90AA1 PAK1 BCL2 | |
| Endothelin | EGR1 EGFR AKT1 PTK2B TRPC6 EDNRA | NCI PID (19) |
| Signaling | GNAI2 YWHAZ MAP3K8 CALM1 YWHAQ | |
| | JUN MAPK3 MAPK1 PRKG1 PIK3R1 EDN1 | |
| | SRC NOS3 SHC1 FOS PRKCA PPARG PRKCZ | |
| | PTPN1 EP300 MAPK8 ADCY1 ADCY5 | |
| | CSNK1A1 GSK3B PTEN GRB2 PRKACA | |
| | MMP1 BCL2 | |
| VEGF | HIF1A CTNNB1 AKT1 PTK2B RHOA CAV1 | KEGG (16) |
| Signaling | CALM1 FYN MAPK3 MAPK1 PIK3R1 SRC | Reactome (18) |
| | ITGB3 NOS3 SHC1 RAC1 PRKCA VEGFA | NCI PID (19) |
| | PTPN1 EPAS1 VHL PTPN11 ROCK1 GSK3B | |
| | GRB2 PRKACA HSP90AA1 PAK1 | |
| Hypoxia and | SMAD4 HIF1A SMAD3 VHL SERPINE1 | Biocarta (17) |
| Oxygen | CXCL12 STAT1 ENG JUN GNB2L1 PIK3R1 | NCI PID (19) |
| Homeostasis | EDN1 MAPK8 NOS1 CDKN2A FOS SP1 | |
| | VEGFA RBX1 EP300 EPAS1 TP53 AKT1 PTEN | |
| | HSP90AA1 BCL2 | |
| Apoptosis | DYNLL1 HSPA5 BIRC4 CTNNB1 AKT1 ILIA | KEGG (16) |
| | ADD1 JUN MAPK3 MAPK1 PIK3R1 NFKB1 | Biocarta (17) |
| | MAPK8 LIMK1 RPS27A PRKCA CHUK RELA | Reactome (18) |
| | TP53 BMX ROCK1 PRKACG PRKACA | NCI PID (19) |
| | PRKACB BCL2 | |
| PDGF | PDGFRB PDGFRA SRC FYN HIF1A AKT1 | Biocarta (17) |
| Pathway | CAV1 STAT3 STAT1 FOS JUN MAPK3 | NCI PID (19) |
| | MAPK1 PIK3R1 PDGFA PDGFB SHC1 RAC1 | |
| | PRKCA PTPN1 LRP1 PTPN11 PTEN GRB2 | |
| Vascular | EDNRA EDN1 CALM1 FGF2 MAPK3 | KEGG (16) |
| Smooth | GUCY1A3 MAPK1 PRKG1 GUCY1A2 | |
| Muscle | ARHGEF12 PRKCA GUCY1B3 PRKCZ RHOA | |
| Contraction | PRKCB1 ADCY5 ADCY1 PPP1CA ROCK1 | |
| | ROCK2 PRKACG PRKACA PRKACB | |
| Vascular | CCL2 CDKN1B CCR1 PTK2B ILIA GNAI2 | NCI PID (19) |
| Inflammation | ADRBK1 CEBPB CYP1B1 STAT1 MAPK1 | |
| | NFKB2 RAC1 PRKCZ STAT3 RHOA CXCL12 | |
| | AKT1 ROCK1 PTEN GRB2 | |
| Nitric Oxide | NOS1 N053 PDE6D CALM1 PDE1C PDE1A | Biocarta (17) |
| Synthesis | GCH1 PDE3A PDE9A PDE2A PRKCA | Reactome (18) |
| | GUCY1A3 GUCY1A2 AKT1 PDE6G GUCY1B2 | |
| | GUCY1B3 HSP90AA1 PDE5A CAV1 | |
| Regulation of | PDGFRB PDGFRA LIMK2 LIMK1 EGFR | KEGG (16) |
| Actin | ARHGEF12 ITGB3 MAPK3 MAPK1 PIK3R1 | |
| Cytoskeleton | PDGFA PDGFB RAC1 RHOA FN1 PPP1CA | |
| | ROCK1 ROCK2 ACTB PAK1 | |
| Angiopoietin | AKT1 FOXO1 STAT3 STAT1 FYN MAPK3 | NCI PID (19) |
| Signaling | MAPK1 PIK3R1 RELA NOS3 SHC1 RAC1 | |
| | MAPK8 FN1 BMX PTPN11 GRB2 PAK1 | |
| Calcium | PDGFRB PDGFRA NOS3 ADCY1 NOS1 | KEGG (16) |
| Signaling | PDE1C PDE1A CAMK2G EGFR PRKACG | |
| Pathway | PRKCA PTK2B TRPC1 PRKACA EDNRA | |
| | PRKACB TBXA2R CALM1 | |
| Cation | NOS1 ADCY1 MAPK3 CXCL12 NOS3 | Biocarta (17) |
| Channel | HSP90AA1 TRPC3 GUCY1A3 TRPC1 TRPC6 | |
| Activity | GUCY1A2 GUCY1B3 EDN1 PRKG1 CAV1 | |
| Thromboxane | TBXA2R NOS3 RAC1 PRKCA EGFR FYN | NCI PID (19) |
| A2 Receptor | ROCK1 SRC AKT1 PRKCZ PRKG1 PRKACA | |
| Signaling | RHOA GNAI2 ADRBK1 | |
| P53 Signaling | CDKN2A TP53 MAPK3 STAT1 AKT1 HIF1A | KEGG (16) |
| Pathway | CTNNB1 PTEN PML HSP90AA1 EP300 | Biocarta (17) |
| | SERPINE1 BCL2 | |
| EGF Pathway | SRC SHC1 GRB2 PTPN11 RPS27A EGFR | Reactome (18) |
| | SH3KBP1 MAPK3 MAPK1 PIK3R1 | |
| FoxO Family | YWHAZ CSNK1A1 RAN CDKN1B YWHAQ | NCI PID (19) |
| Signaling | CTNNB1 AKT1 FOXO1 MAPK8 EP300 | |
| Rho Kinase | SRC ARHGEF12 PIK3R1 RAC1 ROCK1 | Biocarta (17) |
| | MAPK3 AR RHOA RHOG PAK1 | Reactome (18) |
| PPAR | APOE ILK RXRA PPARG LRP1 UBC MMP1 | KEGG (16) |
| Signaling | | |
| Pathway | | |
| Protein | HSPA5 STUB1 HSPA8 RBX1 HSP90AA1 | KEGG (16) |
| Processing in | MAPK8 BCL2 | |
| the ER | | |
| Mitochondrial | PDHA2 PDHAl PDK2 PDK1 DLAT | KEGG (16) |
| Metabolism | Reactome (18) | |
| Fibrosis | MME MMP2 MMP1 | Reactome (18) |
| | | NCI PID (19) |
| Prostacyclin | — | Reactome (18) |
| | | NCI PID (19) |
| Serotonin | — | NCI PID (19) |
| Signaling | | |

The miR-130/301 Family Regulates the Production of Vasoactive Factors in PAECs:

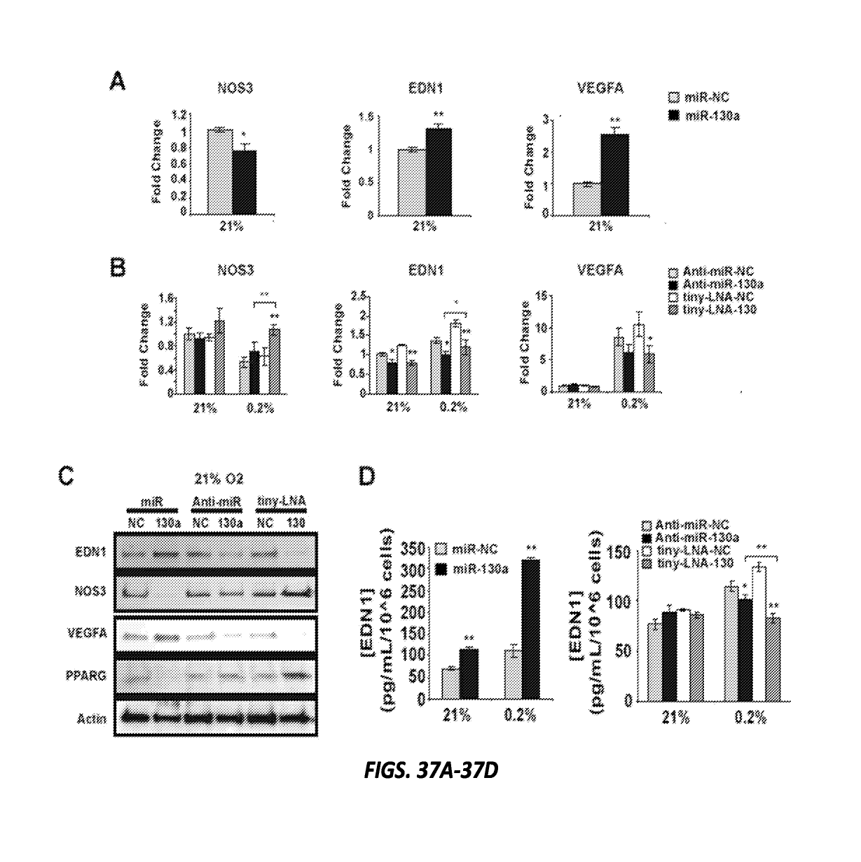

In order to validate experimentally the predicted importance of miR-130/301 on vasomotor tone, we performed gain-of-function and loss-of-function approaches interrogating the regulation of a panel of downstream vasoactive factors known to be modulated in PH. In normoxic (21% O₂) PAECs, forced expression of miR-130a resulted in the intracellular up-regulation of vascular endothelial growth factor-A (VEGFA), and endothelin-1 (EDN1), as well as the down-regulation of endothelial nitric oxide synthase (NOS3) (FIG. 37A, 37C). Additionally, the use of “tinymers,” short locked nucleic acid (LNA), oligonucleotides with selective antisense complementarity to the miR-130/301 family seed sequence 18, allowed for suppression of these miRNAs (as demonstrated in Bertero et al., “Systems-level regulation of microRNA networks by miR-130/301 promotes pulmonary hypertension,” submitted manuscript) and reversed these effects. At the transcript level, this effect was more robust under hypoxic conditions when miR-130/301 family members were endogenously up-regulated (FIG. 37B); however, at the protein level, such an effect was evident at normoxic conditions (FIG. 37C). Notably, tiny-LNA-130 up-regulated these factors to a greater extent than an inhibitor of miR-130a alone, emphasizing the importance of coordinated action by this miRNA family.

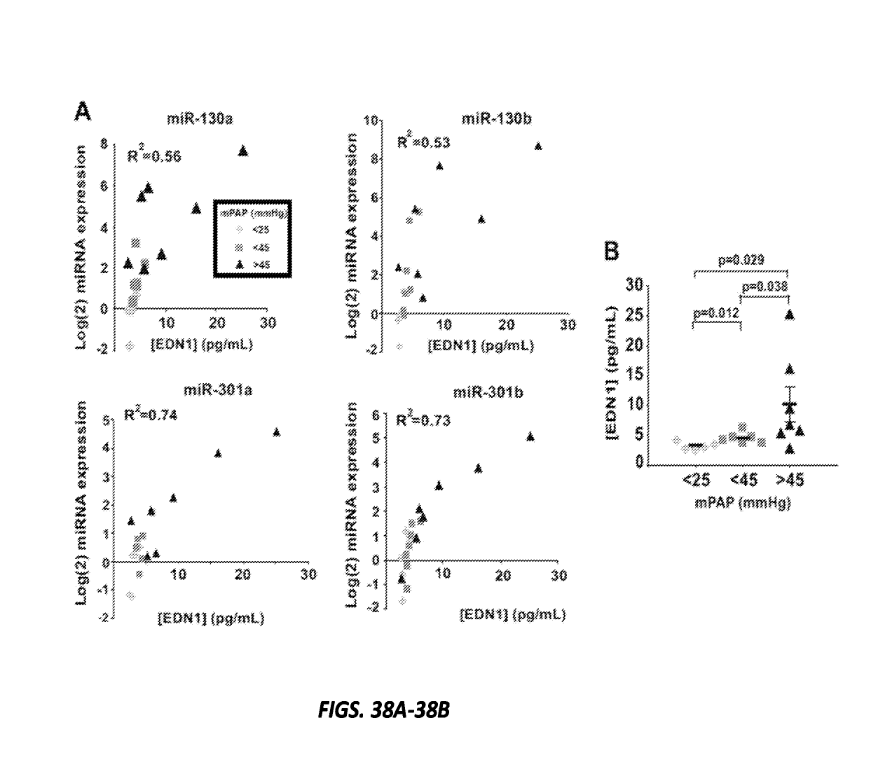

In the case of EDN1, released EDN1 levels were elevated in conditioned media after forced expression of miR-130a, corresponding with the intracellular increase in EDN1 expression. These levels were reciprocally decreased by tinymer inhibition in hypoxia (FIG. 37D). Similarly, in plasma derived from PH patients (demographics described in Bertero et al., “Systems-level regulation of microRNA networks by miR-130/301 promotes pulmonary hypertension,” submitted manuscript), a positive linear correlation was observed between circulating levels of EDN1 and individual miR-130/301 family members (FIG. 38A). Circulating EDN1 levels were also correlated with the hemodynamic severity of disease, as assessed by mean pulmonary artery pressure (mPAP) (FIG. 38B). Taken together, these data suggest that elevated EDN1 expression in PH is associated, at least in part, with the activities of the miR-130/301 family.

The miR-130/301 Family Depends Critically on PPARγ for Control of Vasomotor Tone:

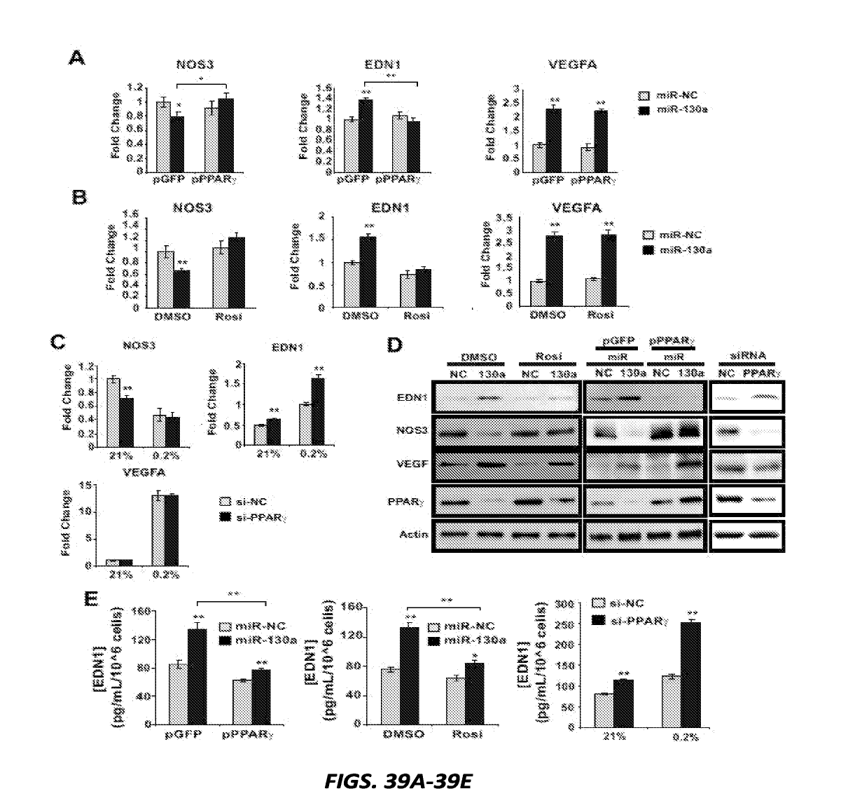

Prior reports have indicated that knockdown of PPARγ induced both PAEC proliferation and EDN1 up-regulation, while forced PPARγ expression under hypoxia attenuated these effects, suggesting a link between these factors (13). Given the up-regulation of EDN1 by miR-130/301 and the known repression of PPARγ by this miRNA family, we investigated whether this relationship, as well was the control of other vasoactive factors, depends on the miR-130/301/PPARγ axis. First, in PAECs, we employed a lentiviral transduction system to express a PPARγ transgene which did not encode for the miR-130/301 family binding site and thus was unaffected by this miRNA family. During forced expression of miR-130a in PAECs, PPARγtransgene expression reversed mIR-130a-mediated alterations in EDN1 and NOS3 (FIG. 39A, 39D). Similarly, in cells treated with the PPARγ agonist rosiglitazone, these miR-130a-dependent changes in EDN1 and NOS3 were reversed (FIG. 39B, 39D). Finally, siRNA knockdown of PPARγ induced expression changes in both EDN1 and NOS3, in the absence of stimulation by the miR-130/301 family (FIG. 39C, 39D). Moreover, levels of secreted EDN1 in conditioned media were consistent with intracellular EDN1 expression in all cases (FIG. 39E). In contrast, in all cases of PPARγ manipulation, VEGFA expression remained unchanged, relative to its expression in cells treated with miR-130a alone. Together, these findings reveal that PPARγserves as a critical link between the miR-130/301 family and EDN1 and NOS3, key effectors controlling pulmonary vasomotor tone. However, other effectors under the control of the miR-130/301 family, such as VEGFA, are regulated independently of PPARγ, highlighting the importance of additional miR-130/301-mediated pathways in other aspects of vascular function.

The miR-130/301/PPARγ/EDN1 Axis Induces Paracrine Activation of STAT3 and Actinomyosin-Dependent Contraction of PASMCs:

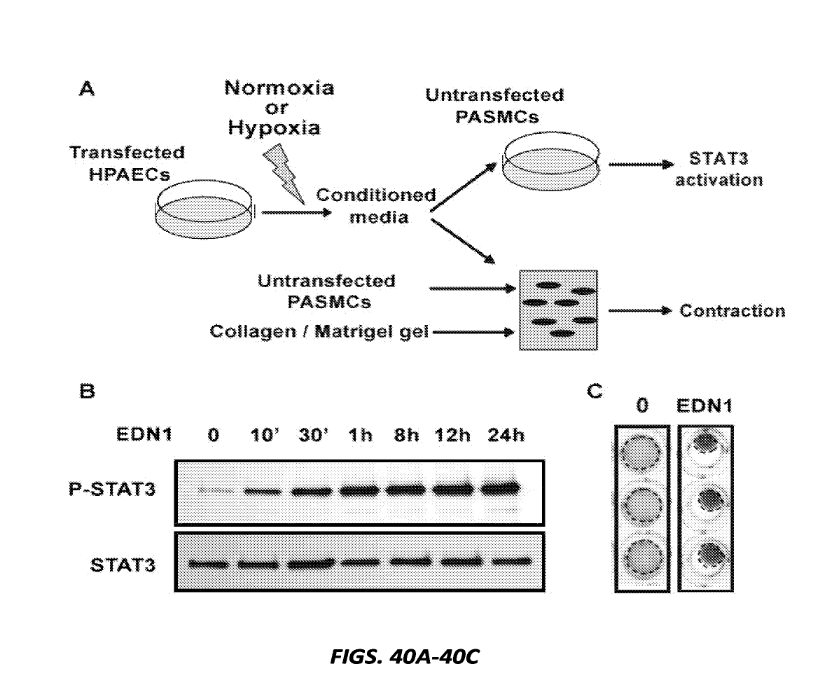

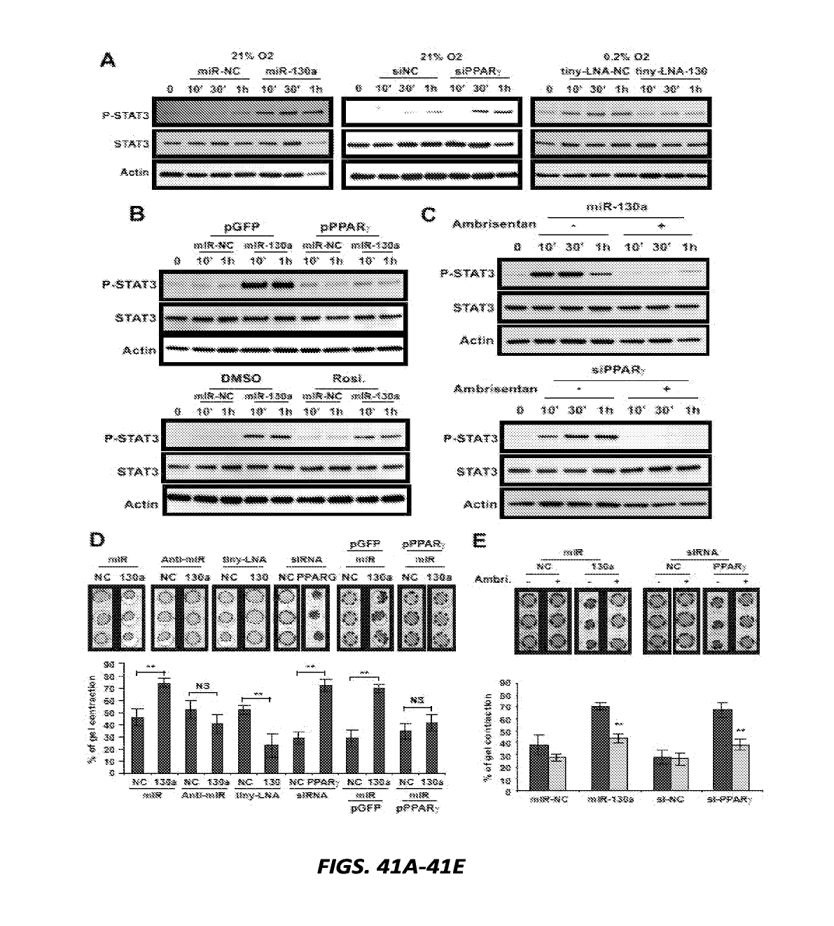

Considering this regulation of EDN1 in PAECs, we hypothesized that the miR-130/301-PPARγ axis controls paracrine activation of contractile vascular function. STAT3, a transcriptional activator that acts in response to cytokine and growth factor stimulation, has been previously linked to proliferation, vasoconstriction, and resistance to apoptosis in PASMCs (21,22). Given the miR-130/301-PPARγ-mediated up-regulation of STAT3 we previously reported in PASMCs (Bertero et al., “Systems-level regulation of microRNA networks by miR-130/301 promotes pulmonary hypertension,” submitted manuscript), we next investigated whether the miR-130/301/PPARγ/EDN1 axis might regulate PASMC function in a STAT3-dependent fashion. We found that direct EDN1 exposure activated STAT3 activity in PASMCs, as demonstrated by increased STAT3 tyrosine phosphorylation (Y705) (FIG. 40B), and increased actinomyosin contraction (FIG. 40C), as reflected by a previously validated in vitro assay of cellular contraction (FIG. 40A) (21,22). Similarly, conditioned media from either PAECs expressing miR-130a or PAECs exposed to siRNA specific for PPARγ increased PASMC-specific STAT3 tyrosine phosphorylation (FIG. 41A) and actinomyosin contraction (FIG. 41D). Conversely, PASMC-specific STAT3 phosphorylation (FIG. 41A) and contraction (FIG. 41D) were abrogated by conditioned media harvested after inhibition of the miR-130/301 family (tiny-LNA-130) in hypoxic PAECs as compared with media derived from hypoxic PAEC control cells transfected with scrambled control (tiny-LNA-NC). Demonstrating the direct importance of PPARγ in such a contractile phenotype, STAT3 phosphorylation (FIG. 41B) and contraction (FIG. 41D) were also reversed in the presence of miR-130a via augmentation of PPARγ activity in PAECs either via forced PPARγtransgene expression or via rosiglitazone exposure. Finally, demonstrating the essential role of EDN1 in this paracrine axis, ambrisentan, a pharmacological antagonist of endothelin receptors, abolished the activation of STAT3 (FIG. 41C) and contraction of PASMCs (FIG. 41E) in the presence of conditioned media from PAECs transfected with either miR-130a or with siRNA specific for PPARγ. Taken together, these data demonstrate that the miR-130/301-PPARγ axis activates PASMC contraction via paracrine stimulation by secreted EDN1.

The miR-130/301-PPARγ Axis Regulates EDN1 Expression In Vivo:

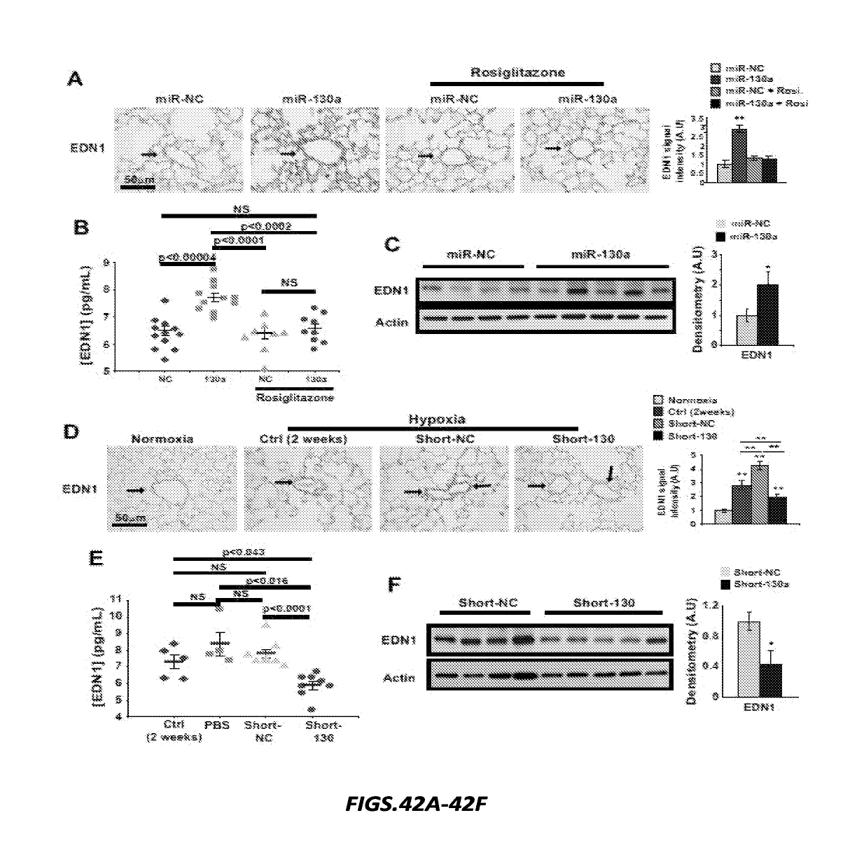

To determine whether this regulatory axis is active in vivo, a series of gain-of-function and loss-of-function experiments were performed to manipulate this miRNA family and its downstream effectors in a mouse model of PH—namely via treatment with the VEGF receptor antagonist SU5416 and chronic hypoxia (25). First, in the presence of SU5416, chronic pulmonary expression of miR-130a in wildtype mice was studied in place of chronic hypoxia. MiRNA was delivered via 4 weekly intrapharyngeal injections of liposomally encapsulated miR-130a oligonucleotide mimics, as adapted from prior protocols (22). As we previously described, this protocol resulted in the up-regulation of miR-130a, with subsequent PPARγ suppression, in whole lung tissue and in small pulmonary vessels, thus leading to increased vascular STAT3 phosphorylation, vascular remodeling, and increased right ventricular systolic pressure (RVSP), a hemodynamic surrogate for pulmonary artery pressure (Bertero et al., “Systems-level regulation of microRNA networks by miR-130/301 promotes pulmonary hypertension,” submitted manuscript). Here, we found that EDN1 was up-regulated under these conditions in both small pulmonary vessels and plasma (FIGS. 42A-42C), an effect which was reversed by PPARγ activation via rosiglitazone.

Conversely, inhibition of the miR-130/301 family in the pulmonary vasculature was achieved by pharmacologic means. As we previously described (Bertero et al., “Systems-level regulation of microRNA networks by miR-130/301 promotes pulmonary hypertension,” submitted manuscript) after two weeks of treatment with hypoxia+SU5416, RVSP elevation was confirmed, followed by two additional weeks of hypoxia+SU5416 in the setting of 3 serial intrapharyngeal injections of either a fully-modified, short anti-miR complementary to the seed sequence (e.g. “shortmer”) or scrambled control. In that setting, we previously found a de-repression of PPARγ, a decrease in phosphorylated STAT3, and protection against the histologic and hemodynamic consequences of PH. Correspondingly, EDN1 expression was decreased in both vascular tissue and plasma (FIGS. 42D-42F). Thus, considering both gain-of-function and loss-of-function experimentation, we conclude that chronic induction of endogenous miR-130/301 family members is necessary and sufficient to increase pathogenic expression of EDN1 in vivo, and such robust actions depend, at least in part, upon hierarchical control of PPARγ.

### Discussion

In this study, we expanded our analysis of PH gene network architecture to predict and experimentally validate the importance of the miR-130/301 family and its direct target PPARγ in specific control of pulmonary vascular cell crosstalk and vasomotor tone. Specifically, we found that, in addition to vascular proliferation, this family regulates a panel of vasomotor effectors throughout the vasculature, allowing it to modulate intercellular communication between the disparate cell types involved in PH. Moreover, we validated the intricate control over EDN1 which, when up-regulated by miR-130/301, induces STAT3 activation and vasoconstriction in PASMCs. Taken together, these data further characterize the miR-130/301 family as master regulators of a hierarchy of pathogenic pathways relevant to the pulmonary vasculature and PH. Accordingly, our study provides critical experimental support that complex miRNA-based relationships deeply embedded in the PH disease gene network can be deciphered accurately from available molecular disease maps.

While the pool of known PH-relevant genes and triggers is growing rapidly, our understanding of any type of a global regulatory structure has been limited in the context of PH. Guided by network theory, we now provide proof of extensive control of PH by miR-130/301 that extends beyond vascular proliferation alone (Bertero et al., “Systems-level regulation of microRNA networks by miR-130/301 promotes pulmonary hypertension,” submitted manuscript) and impacts vasoconstriction. While it is known that diseased PAECs secrete a variety of growth and vasoactive factors, the extent of their contribution to PASMC dysfunction is not well understood (2). In that vein, our data position the miR-130/301 family at a key checkpoint coordinating the intercellular crosstalk between PAECs and PASMCs via controlling released vasoactive factors. Additionally, while our research has primarily focused on the PPARγ-dependent actions of the miR-130/301 family, we have also demonstrated its PPARγ-independent regulation of other vasoactive factors such as VEGF, suggesting that this miRNA family may have other direct targets, outside of PPARγ, with immediate relevance to this disease. Future applications of advanced network theory may prove invaluable in elucidating the complete systems-wide control of PH by the miR-130/301 family even beyond proliferation and vasomotor tone. Such analyses may also establish the importance of this miRNA family in other disease conditions and could identify critical miRNA-based commonalities across the spectrum of cardiovascular diseases and human disease in general that otherwise have been missed by conventional scientific approaches.

From a clinical perspective, the up-regulation of circulating miR-130/301 in PH has been confirmed by multiple sources (including our group (Bertero et al., “Systems-level regulation of microRNA networks by miR-130/301 promotes pulmonary hypertension,” submitted manuscript) and Wei and colleagues (27)), thus establishing this miRNA family as potential PH biomarkers. The strong correlation between miR-130/301 family expression and the released levels of EDN1 further supports the notion of circulating miR-130/301 levels as prognostic markers of PH severity. Moreover, given the miR-130/301-dependence of EDN1 up-regulation, it may be reasonable to expect that miR-130/301 plasma levels increase even before the initiation of vasoconstriction and vascular hyperplasia—thus allowing this this miRNA family to be detected in advance of symptom onset. This type of temporal separation between circulating miRNA expression and the expression more traditional biomarkers has been demonstrated in other contexts, such as the early release of inflammatory miRNA into circulation following strenuous exercise (28), and may be useful in detecting disease before it has become severe. Finally, these results suggest that the miR-130/301 family could be a powerful therapeutic target, potentially when used in combination with an endothelin receptor antagonist, or other vasodilatory medications.

In summary, by extending our analysis and validation of disease gene network architecture, we have broadened our understanding of this miRNA family in the control of vascular paracrine signaling and vasomotor tone during PH. These results set the stage for future work to provide structure to the growing pool of miRNA, genes, and pathways that make up the PH diseaseome and to apply such finding for diagnostic and therapeutic gains.

### Methods

Cell Culture and Reagents:

Primary human pulmonary arterial endothelial cells (PAECs) were purchased and propagated in EGM-2 cell culture media (Lonza). Experiments were performed at passages 3 to 6. Primary human pulmonary arterial smooth muscle cells (PASMCs) were purchased and propagated in SmGM-2 cell culture media (Lonza). Experiments were performed at passages 3 to 9. HEK293T cells (American Type Culture Collection) were used and cultivated in DMEM containing 10% fetal bovine serum (FBS). Cells were cultured at 37° C. in a humidified 5% CO2 atmosphere. A modular hypoxia chamber at 0.2% oxygen, 94.8% nitrogen and 5% carbon dioxide was used during 24 h for hypoxic conditions. Cultured cells were exposed to recombinant human EDN1 at 1 nM. Rosiglitazone (Sigma) was used at 10 mM. Ambriesantan was used at 10 μM.

Lentivirus Production:

HEK293T cells were transfected using Lipofectamine 2000 (Life Technologies) with lentiviral plasmids along with packaging plasmids (pPACK, System Biosciences), according to the manufacturer's instructions. Virus was harvested, sterile filtered (0.45 um), and utilized for subsequent infection of PAECs or PASMCs (24-48 hour incubation) for gene transduction.

Oligonucleotides and Transfection:

Pre-miRNA oligonucleotides (pre-miR-130a and negative control pre-miR-NC1 and premiR-NC2), anti-miRNAs oligonucleotides (anti-miR-130a and anti-miR-NC), and custom designed tiny LNA oligonucleotides (tiny-130: 5′-ATTGCACT-3′ (SEQ ID NO: 3), and tiny-NC 5′-TCATACTA-3′ (SEQ ID NO: 51)) were purchased from Life Technologies/Ambion, ThermoScientific/Dharmacon, and Exiqon, respectively. siRNAs for PPARγ and scrambled control were purchased from Santa Cruz Biotechnology. PAEC and PASMC were plated and transfected 24 h later at 70-80% confluence using pre-miRNA (5 nM), anti-miRNA (20 nM), tiny-LNA (20 nM) or siRNA (25 nM) and Lipofectamine 2000 reagent (Life Technologies), according to the manufacturers' instructions.

Plasmids:

Coding sequence of PPARγ (BC006811) was amplified by polymerase chain reaction (PCR) using high-fidelity polymerase Phusion (Thermo Fisher Scientific) from an MGC cDNA clone (clone ID: 3447380) and inserted in the pCDH-CMV-MCS-EF1-copGFP (System Biosciences) using NheI and NotI restrictions sites. The lentiviral parent vector expressing GFP was used as a control. Stable expression of these constructs in PAECs or PASMCs was achieved by lentiviral transduction. All cloned plasmids were confirmed by DNA sequencing.

Quantification of Endothelin-1 by ELISA:

Twenty-four hours after transfection PAECs were washed in PBS, and medium was replaced by media without serum. Twenty-four hour later conditioned media from PAEC transfected as indicated were collected and endothelin-1 was measured by ELISA kit (Enzo biosciences) according to the manufacturer's protocol. Human plasma sample (see below) and mouse plasma were also tested for endothelin-1 level by ELISA.

Contraction and Co-Culture Assays:

50000 PASMC were embedded in 100 ul of matrix gel as previous described (Gagiolli et al) and plated into well of a 96 well plate. After 1 h at 37° C., these matrices were overlaid with 100 ul of conditioned PAECs serum-free medium (transfected with siRNA, miRNA or anti-miRNA and supplemented with Ambriesentan 10 uM). Every 12 h, medium was changed and at day 4 the gels were photographed and the relative diameter of the well and the gel were measured using ImageJ. The percentage contraction was calculated using the formula 100*(well diameter−gel diamter)/well diameter. For the co-culture experiment, conditioned media from PAEC transfected as indicated were applied on PASMCs or Fibroblast previously starved (24 h). Cells were collected at different time and analyzed by western blotting.

Messenger RNA Extraction:

Cells were homogenized in 2 ml of QiaZol reagent (Qiagen). Total RNAs including small RNAs were extracted using the miRNeasy kit (Qiagen) according to the manufacturer's instructions. Total RNA concentration was determined using a ND-1000 micro-spectrophotometer (NanoDrop Technologies).

Quantitative RT-PCR of Messenger RNAs:

Messenger RNAs were reverse transcribed using the Multiscript RT kit (Life Technologies) to generate cDNA. cDNA was amplified via fluorescently labeled Taqman primer sets using an Applied Biosystems 7900HT Fast Real Time PCR device, as we previously described (29). Fold-change of RNA species was calculated using the formula (2^{-ΔΔCt}), normalized to actin expression.

Immunoblotting and Antibodies:

Cells were lysed in RIPA buffer (Santa Cruz Biotechnology) and the protein concentration determined using a Bradford assay (Biorad). Protein lysate (40 mg) were resolved by SDS-PAGE and transferred onto a PVDF membrane (Biorad). Membranes were blocked in 5% non-fat milk in TN buffer (50 mM Tris-HCl pH 7.4, 150 mM NaCl) and incubated in the presence of the primary and then secondary antibodies. After washing in TN buffer containing 0.1% Tween, immunoreactive bands were visualized with the ECL system (Amersham Biosciences). Primary polyclonal or monoclonal antibodies to EDN1 (sc 21625), NOS3 (sc 654), PPARγ (sc 7273) and VEGFA (sc 152) were obtained from Santa Cruz Biotechnology. Primary antibodies for STAT3 (#9139) and P-STAT3 (Tyr-705; #9145) were purchased from Cell Signaling. Primary antibodies against actin (ab 3280), was obtained from Abcam. Appropriate secondary antibodies (anti-rabbit and anti-mouse) coupled to HRP were used (Santa Cruz Biotechnology).

Human Plasma Sampling:

To collect blood from subjects from the main pulmonary artery, clinically indicated right heart catheterization procedures were performed by standard protocol via a right internal jugular approach under fluoroscopic guidance. The catheter was positioned into the main pulmonary artery, as confirmed by fluoroscopy and hemodynamic waveforms. Blood was then drawn from the distal catheter port and collected in standard vacutainer tubes with K+-EDTA anticoagulant. Plasma was extracted after standard centrifugation of blood, followed by storage at −80° C.

Forced Expression of miR-130a in Mouse Lung:

Eight-week-old mice (C57Bl6) were injected with SU5416 (20 mg/kg; Sigma-Aldrich), followed by 4 intrapharyngeal injections (once by week) of 1 nmol of miR-control (pre-miR-NC) or miR-130a (pre-miR-130a) mixed in 100 uL PBS solution containing 5% Lipofectamine 2000 (Life Technologies). Three days after the last injection, right heart catheterization was performed as previously described (11), followed by harvest of lung tissue for RNA extraction or paraffin embedding, as described above. Rosiglitazone (Cayman Chemicals) was dissolved in 0.25% carboxymethyl cellulose medium viscosity aqueous solution. This rosiglitazone solution versus vehicle control was delivered daily (20 mg/kg/day) by oral gavage to mice for 3 weeks.

Inhibition of the miR-130/301 Family in Mouse Lung:

Eight-week-old mice (C57Bl6) were injected with SU5416 (20 mg/kg; Sigma-Aldrich), followed by exposure to normobaric hypoxia (10% O2; OxyCycler chamber, Biospherix Ltd, Redfield, N.Y.) for 2 weeks. After two weeks and confirmation of PH development in five mice (right heart catheterization), mice were further treated with 3 intrapharyngeal injections (every 4 days) of control or miR-130/301 shortmer oligonucleotides, designed as fully modified antisense oligonucleotides complementary to the seed sequence of the miR-130/301 miRNA family (10 mg/kg; Regulus). Specifically, the control and miR-130/301 shortmer oligonucleotides were non-toxic, lipid-permeable, high-affinity oligonucleotides. The miR-130/301 shortmer carried a sequence complementary to the active site of the miR-130/301 miRNA family, containing a phosphorothioate backbone and modifications (fluoro, methoxyethyl, and bicyclic sugar) at the sugar 2′ position. Three day after the last injection, right heart catheterization was performed followed by harvest of lung tissue for RNA extraction or paraffin embedding, as described above.

Tissue Harvest:

After physiological measurements, blood was extracted by cardiac puncture for future analyses (hematocrit, plasma extraction). By direct right ventricular puncture, the pulmonary vessels were gently flushed with 1 cc of saline to remove the majority of blood cells, prior to harvesting cardiopulmonary tissue. The heart was removed, followed by dissection and weighing of the right ventricle (RV) and of the left ventricle+septum (LV+S). Organs were then harvested for histological preparation or flash frozen in liquid N2 for subsequent homogenization and extraction of RNA and/or protein. To further process lung tissue specifically, prior to excision, lungs were flushed with PBS at constant low pressure (˜10 mmHg) via right ventricular cannulation, followed by tracheal inflation of the left lung with 10% neutral-buffered formalin (Sigma-Aldrich) at a pressure of ˜20 cm H2O. After excision and 16 hours of fixation in 10% neutral-buffered formalin at 25° C., lung tissues were paraffin-embedded via an ethanol-xylene dehydration series, before being sliced into 5 μm sections (Hypercenter XP System and Embedding Center, Shandon).

Immunohistochemistry of Mouse Lung:

Lung sections (5 um) were deparaffinized and high temperature antigen retrieval was performed followed by blocking in TBS/BSA 5%, 10% goat serum and exposure to primary antibody and biotinylated secondary antibody (Vectastain ABC kit, Vector Labs). In most cases, color development was achieved by adding streptavidin-biotinylated alkaline phosphatase complex (Vector Labs) followed by Vector Red alkaline phosphatase substrate solution (Vector Labs). Levamisole was added to block endogenous alkaline phosphatase activity (Vector Labs). Pictures were obtained using an Olympus Bx51 microscope (40× objective). Small pulmonary vessels (<100 μm diameter) present in a given tissue section (>10 vessels per section) that were not associated with bronchial airways were selected for analysis (N>5 animals per group). Intensity of staining was quantified using ImageJ software (NIH).

Statistics

Cell culture experiments were performed at least three times and at least in triplicate for each replicate. The number of animals in each group was calculated to measure at least a 20% difference between the means of experimental and control groups with a power of 80% and standard deviation of 10%. The number of unique patient samples for this study was determined primarily by clinical availability. RT-qPCR on human plasma, in situ expression/histologic analyses of both mouse and human tissue, and pulmonary vascular hemodynamics in mice were performed in a blinded fashion. Numerical quantifications for in vitro experiments using 3D cultured cells or in situ quantifications of transcript/miRNA expression represent mean±standard deviation (SD). Numerical quantifications for physiologic experiments using mice or human reagents represent mean±standard error of the mean (SEM). Immunoblot images are representative of experiments that have been repeated at least three times. Micrographs are representative of experiments in each relevant cohort of mice. Paired samples were compared by Student's t test. Comparison of multiple samples was performed by ANOVA followed by Student Newman-Keuls post hoc testing. Correlation analyses were performed by Pearson correlation coefficient calculation, as previously described in reference 31.

### References for Example 3

1. Schermuly R T, Ghofrani H A, Wilkins M R, Grimminger F. Mechanisms of disease: Pulmonary arterial hypertension. Nat Rev Cardiol. 2011; 8:443-455.
2. Humbert M, Montani D, Perros F, Dorfmuller P, Adnot S, Eddahibi S. Endothelial cell dysfunction and cross talk between endothelium and smooth muscle cells in pulmonary arterial hypertension. Vascul Pharmacol. 2008; 49:113-118.
3. Giaid A, Yanagisawa M, Langleben D, et al. Expression of endothelin-1 in the lungs of patients with pulmonary hypertension. N Engl J Med. 1993; 328:1732-1739.
4. Markewitz B A, Farrukh I S, Chen Y, Li Y, Michael J R. Regulation of endothelin-1 synthesis in pulmonary arterial smooth mucle cells. Effects of transforming growth factor-beta and hypoxia. 2001; 49:200-206.
5. Pulido T, Adzerikoho I, Channick R N, et al. Macitentan and morbidity and mortality in pulmonary arterial hypertension. N Engl J Med. 2013; 369:809-818.
6. Chua B H, Krebs C J, Chua C C, Diglio C A. Endothelin stimulates protein synthesis in smooth muscle cells. Am J Physiol. 1992; 262:E412-416.
7. Stewart D J, Levy R D, Cernacek P, Langleben D. Increased plasma endothelin-1 in pulmonary hypertension: Marker or mediator of disease? Ann Intern Med. 1991; 114:464-469.
8. Bando K, Vijayaraghavan P, Turrentine M W, et al. Dynamic changes of endothelin-1, nitric oxide, and cyclic gmp in patients with congenital heart disease. Circulation. 1997; 96:11-346-351.
9. Chen Y F, Oparil S. Endothelin and pulmonary hypertension. J Cardiovasc Pharmacol. 2000; 35: S49-53.
10. Rubin L J. Endothelin receptor antagonists for the treatment of pulmonary artery hypertension. Life Sci. 2012; 91:517-521.
11. Kim E K, Lee J H, Oh, Y M, Lee Y S, Lee S D. Rosiglitazone attenuates hypoxia-induced pulmonary arterial hypertension in rats. Respirology. 2010; 15:659-668.
12. Kang B Y, Kleinhenz J M, Murphy T C, Hart C M. The PPARgamma ligand rosiglitazone attenuates hypoxia-induced endothelin signaling in vitro and in vivo. Am J Physiol Lung Cell Mol Physiol. 2011; 301:L881-891.
13. Kang B Y, Park K K, Green D E, et al. Hypoxia mediates mutual repression between microrna-27a and ppargamma in the pulmonary vasculature. PloS One. 2013; 8:e79503.
14. Din S, Sarathchandra P, Yacoub M H, Chester A H. Interaction between bone morphogenic proteins and endothelin-1 in human pulmonary artery smooth muscle. Vascul Pharmacol. 2009; 51:344-349.
15. Barabasi A L, Gulbahce N, Loscalzo J. Network medicine: a network-based approach to human disease. Nat Rev Genet. 2011; 12:56-68.
16. Kanehisa M, Goto S. KEGG: Kyoto Encyclopedia of Genes and Genomes. Nucleic Acids Res. 2000; 28:27-30.
17. Nishimura D. BioCarta. Biotechnol Softw I J. 2001; 2(3):117-120.
18. Matthews L, Gopinath G, Gillespie M, et al. Reactome knowledgebase of human biological pathways and processes. Nucleic Acids Res. 2008; 37:D619-22.
19. Schaefer C F, Anthony K, Krupa S, et al. PID: The Pathway Interaction Database. Nucleic Acids Res. 2009; 37:D674-9.
20. Obad S, dos Santos C O, Petri A, et al. Silencing of microRNA families by seed-targeting tiny LNAs. Nat Genet. 2011; 43:371-378.
21. Paulin R, Courboulin A, Meloche J, Mainguy V, de la Roque E D, Saksouk N. Signal transducers and activators of transcription-3/piml axis plays a critical role in the pathogenesis of human pulmonary arterial hypertension. Circulation. 2011; 123:1205-1215.
22. Courboulin A, Paulin R, Giguere N J, et al. Role for mir-204 in human pulmonary arterial hypertension. J Exp Med. 2011; 208:535-548.
23. Gaggioli C, Hooper S, Hidalgo-Carcedo C, et al. Fibroblast-led collective invasion of carcinoma cells with differing roles for rhogtpases in leading and following cells. Nat Cell Biol. 2007; 9:1392-1400.
24. Sanz-Moreno V, Gaggioli C, Yeo M, et al. Rock and jak1 signaling cooperate to control actomyosin contractility in tumor cells and stroma. Cancer Cell. 2011; 20:229-245.
25. Ciuclan L, Bonneau O, Hussey M, et al. A Novel murine model of severe pulmonary arterial hypertension. Am J Respir Crit Care Med. 2011; 184:1171-82.
26. Hampl V, Herget J. Role of nitric oxide in the pathogenesis of chronic pulmonary hypetension. Physiol Rev. 2000; 80:1337-13372.
27. Wei C, Henderson H, Spradley C, Li L, Kim I K, Kumar S, et al. Circulating miRNAs as potential marker for pulmonary hypertension. PLoS One. 2013; 8:e64396.
28. Baggish A L, Hale A, Weiner R B, et al. Dynamic regulation of circulating microRNA during acute exhaustive exercise and sustained aerobic exercise training. 2011; 589:3983-3994.
29. Parikh V N, Jin R C, Rabello S, et al. MicroRNA-21 Integrates Pathogenic Signaling to Control Pulmonary Hypertension: Results of a Network Bioinformatics Approach. Circulation. 2012; 125:1520-1532.
30. Kajstura J, Rota M, Hall S R, et al. Evidence for human lung stem cells. N Engl J Med. 2011; 364:1795-1806.
31. Kim J, Kang Y, Kojima Y, et al. An endothelial apelin-FGF link mediated by miR-424 and miR-503 is disrupted in pulmonary arterial hypertension. Nat Med. 2013; 19:74-82.

### Example 4: The miR-130/301 Family Modulates Matrix Deposition

Cell Culture and Reagents: Primary human pulmonary arterial adventitial fibroblast cells (PAAFs) were purchased and propagated in FGM cell culture media (Lonza). Experiments were performed at passages 3 to 6. Primary human pulmonary fibroblast cells (PFs) were purchased and propagated in FGM cell culture media (Lonza). Primary human pulmonary arterial endothelial cells (PAECs) were purchased and propagated in EGM-2 cell culture media (Lonza). Experiments were performed at passages 3 to 6. Primary human pulmonary arterial smooth muscle cells (PASMCs) were purchased and propagated in SmGM-2 cell culture media (Lonza). Experiments were performed at passages 3 to 9. HEK293T cells (American Type Culture Collection) were used and cultivated in DMEM containing 10% fetal bovine serum (FBS). Cells were propagated in coated collagen plastic (50 ug/mL) at 37° C. in a humidified 5% CO₂ atmosphere. Collagen-coated hydrogel were purchased.

3D cell culture: Protocol was adapted from previously published protocol (Aragona M et al, 2013 Cell). Cells were embedded in a mixture of Growth Factor Reduced Matrigel (BD Biosciences) and Collagen-I (BD Biosciencres). Collagen-I solution was neutralized on ice with 0.1M NaOH in PBS and adjusted with 0.1N HCl to bring the pH of the solution to 7.5. The Collagen-I solution was then mixed on ice with Matrigel to obtain a final concentration of 1.2 mg/ml (soft matrices) or 3 mg/ml (stiff matrices). Cells were trypsinized, counted, resuspended in growth medium; then, 1 volume of cells was mixed with 1 volume of the ECM mix. 6-well plated were precoated with 1 ml of cellfree 50% medium/50% ECM, and left in the incubator until gelled; then, cells were seeded in drop on top of the pregelled ECM. After gelling, wells were supplemented with normal growth medium, which was changed every 2 days during the experiments.

Oligonucleotides and Transfection: Pre-miRNA oligonucleotides (pre-miR-130a, negative control pre-miR-NC1 and premiR-NC2) and custom designed tiny LNA oligonucleotides (tiny-130: 5′-CCACTCCC-3′ (SEQ ID NO: 52), and tiny-NC 5′-TTCTCCTAAT-3′ (SEQ ID NO: 54)) were purchased from Life Technologies/Ambion and Exiqon, respectively. siRNAs for PPARγ and OCT4/POU5F1 were purchased from Santa Cruz Biotechnology. siRNA for TIMP2 was purchased from Life Technologies. On Target Plus siRNAs for YAP1, TAZ (WWTR1), LRP8 and scrambled control were purchased from ThermoScientific/Dhermacon. PAAF, PF, PAEC and PASMC were plated in collagen-coated dish (50 ug/mL) and transfected 24 h later at 70-80% confluence using pre-miRNA (5 nM), tiny-LNA (20 nM) or siRNA (25 nM) and Lipofectamine 2000 reagent (Life Technologies), according to the manufacturers' instructions. 8 hours after transfection cells were trypsinized and re-plated in the different hydrogel/3D-matrix.

Plasmids: 3′UTR sequence from TIMP2 and LRP8 were purchased as gene fragment block (IDT) and cloned in the pSI-CHECK-2 vector (Promega) downstream of the Renilla luciferase using XhoI and NotI restrictions sites. Mutagenesis of the putative miR-130-family binding sites were performed using Gene fragment blocks containing mutated nucleotide in position 2, 4 and 6 in the “seed” biding site (IDT). Coding sequence of PPARγ (BC006811) was amplified by polymerase chain reaction (PCR) using high-fidelity polymerase Phusion (Thermo Fisher Scientific) from an MGC cDNA clone (clone ID: 3447380) and inserted in the pCDH-CMV-MCS-EF1-copGFP (System Biosciences) using NheI and NotI restrictions sites. The lentiviral parent vector expressing GFP was used as a control. Stable expression of these constructs in PAAFs, PFs, PAECs or PASMCs was achieved by lentiviral transduction. All cloned plasmids were confirmed by DNA sequencing.

Lentivirus production: HEK293T cells were transfected using Lipofectamine 2000 (Life Technologies) with lentiviral plasmids along with packaging plasmids (pPACK, System Biosciences), according to the manufacturer's instructions. Virus was harvested, sterile filtered (0.45 um), and utilized for subsequent infection of PAAFs, PFs, PAECs or PASMCs (24-48 hour incubation) for gene transduction.

miRNA target validation by luciferase assay: Adapted from our previously published protocol, HEK293T cells were plated in 96-well plates and transfected with 200 ng of pSICHECK-2 constructs and 5 nM of pre-miRNAs using Lipofectamine 2000 (Life Technologies). The medium was replaced 8 hours after transfection with fresh medium containing 10% FCS, L-glutamine. 48 hours after transfection, firefly and Renilla Luciferase activities were measured using the Dual-Glo™ Luciferase assay (Promega).

Messenger RNA and miRNA extraction: Cells were homogenized in 1 ml of QiaZol reagent (Qiagen). Total RNAs including small RNAs were extracted using the miRNeasy kit (Qiagen) according to the manufacturer's instructions. Total RNA concentration was determined using a ND-1000 micro-spectrophotometer (NanoDrop Technologies).

Quantitative RT-PCR of mature miRNAs: Mature miRNA expression was evaluated using TaqMan MicroRNA Assays (Life Technologies/Applied Biosystems) and the Applied Biosystems 7900HT Fast Real Time PCR device (Life Technologies/Applied Biosystems). Expression levels were normalized to RNU48 or snoR55 for human or mouse experiments, respectively, and calculated using the comparative Ct method (2^{-Ct}).

Quantitative RT-PCR of messenger RNAs: Messenger RNAs were reverse transcribed using the Multiscript RT kit (Life Technologies) to generate cDNA. cDNA was amplified via fluorescently labeled Taqman primer sets using an Applied Biosystems 7900HT Fast Real Time PCR device. Fold-change of RNA species was calculated using the formula (2^{-Ct}) normalized to actin expression.

Immunoblotting and antibodies: Cells were lysed in Laemmeli buffer. Protein lysate were resolved by SDS-PAGE and transferred onto a PVDF membrane (Biorad). Membranes were blocked in 5% non-fat milk in TN buffer (50 mM Tris-HCl pH 7.4, 150 mM NaCl) or 5% BSA in TN buffer and incubated in the presence of the primary and then secondary antibodies. After washing in TN buffer containing 0.1% Tween, immunoreactive bands were visualized with the ECL system (Amersham Biosciences). A primary antibody to YAP/TAZ ( ) was obtained from Cell Signaling. Primary antibody to LRP8 (sc 21625), TIMP2 (sc 79) and Actin (ab 3280) were obtained from Abcam. Primary antibody to were obtained from Santa Cruz Biotechnology. A primary antibodies for MMP2 (#9139) was purchased from Millipore. Appropriate secondary antibodies (anti-rabbit and anti-mouse) coupled to HRP were used (Santa Cruz Biotechnology).

Zymography: 10 μL of concentrated (Amicon Ultra 3K, Millipore) conditioned media was loaded on 10% SDS-polyacrylamide gels containing type-I collagen (Biorad). Following electrophoresis, proteins were renatured by incubating gels in 2.5% Triton X-100 for 1 h at 37° C. Gels were then washed three times in distilled water, and incubated in substrate buffer (50 mM Tris pH 7.4 and 5 mM CaCl2) at 37° C. for 24 h with gentle shaking. Gels were stained with 0.1% Coomassie blue R-250 (Sigma) and destained in 7% acetic acid. Enzymatic activities appear as cleared bands in a dark background.

Tissue harvest: After physiological measurements, blood was extracted by cardiac puncture for future analyses (hematocrit, plasma extraction). By direct right ventricular puncture, the pulmonary vessels were gently flushed with 1 cc of saline to remove the majority of blood cells, prior to harvesting cardiopulmonary tissue. The heart was removed, followed by dissection and weighing of the right ventricle (RV) and of the left ventricle+septum (LV+S). Organs were then harvested for histological preparation or flash frozen in liquid N2 for subsequent homogenization and extraction of RNA and/or protein. To further process lung tissue specifically, prior to excision, lungs were flushed with PBS at constant low pressure (˜10 mmHg) via right ventricular cannulation, followed by tracheal inflation of the left lung with 10% neutral-buffered formalin (Sigma-Aldrich) at a pressure of ˜20 cm H₂O. After excision and 16 hours of fixation in 10% neutral-buffered formalin at 25° C., lung tissues were paraffin-embedded via an ethanol-xylene dehydration series, before being sliced into 5 μm sections (Hypercenter XP System and Embedding Center, Shandon).

Lung collagen determination: Protocol was adapted from previous published protocol (Hu et al, 2012 Cell). Mice lung were weighed, minced, and incubated in 0.5 M acetic acid at 4 C. After overnight digestion, the acetic acid-soluble and insoluble fractions were isolated by centrifugation. The soluble fraction was stored at −80 C, while the insoluble fraction was digested by overnight incubation in 6M hydrochloric acid at 85 C. Concentrations of soluble and insoluble (gelatinous) collagen fractions were determined using a Sircol Soluble Collagen Assay Kit (Biocolor) with a colorimetric reaction (measured at 550 nm). Values were converted into collagen amounts (mg/ml) using a provided collagen reference standard.

In situ hybridization: The protocol for in situ hybridization for miRNA detection was based on a prior report (6). Specifically, 5 mm sagittal lung sections were probed using a 3′ fluorescein isothiocyanate (FITC) labeled miRCURY LNA hsa-miR-130a detection probe (Exiqon). The miRCURY LNA scramble-miR probe was used as negative control. Following proteinase K digestion (Sigma) for 15 minutes at 25° C., lungs were formaldehyde-(4% formaldehyde, Sigma) and EDC-fixed [300 mM NaCl, 0.1 M 1-methylimidazole (Sigma)] before inactivation of endogenous enzymes by acetylation buffer [873 uL of triethanolamine (Sigma) and 375 uL acetic anhydride (Fisher) in 75 ml distilled water]. Probe annealing (25 nM LNA probe) was performed in hybridization buffer (Sigma, H7782) for 16 hours at RNA-Tm-22° C. (62° C.). Following serial washes with 2×SSC, 1×SSC and 0.5×SSC (Sigma) at 62° C., immunolabeling was performed with an anti-FITC horseradish peroxidase-conjugated secondary antibody for 1 hour at 25° C. (1:800; DAKO). For detection, tyramide amplification was performed with the Individual Indirect Tyramide Reagent (PerkinElmer) followed by application of the Neutravidin Alkaline Phosphatase detection system (1:200; Thermo Scientific), per the manufacturers' instructions. Visualization was performed with the chromagen Nitro blue tetrazolium chloride/5-Bromo-4-chloro-3-indolyl phosphate (NBT/BCIP, Roche), and positive staining was evident by a purple/dark-purple color. MiR-130a expression was quantified in the vascular wall of 15-20 resistance pulmonary arteries (<100 μm external diameter in rodents and <200 um external diameter in humans) using ImageJ software (NIH).

Picrosirius Red coloration and quantification: Picrosirius red analysis was achieved through the use of 5 mm paraffin sections stained with 0.1% picrosirius red (Direct Red80, Sigma) and counterstained with Weigert's hematoxylin to reveal fibrillar collagen. The sections were then serially imaged using with an analyzer and polarizer oriented parallel and orthogonal to each other. Microscope conditions (lamp brightness, condenser opening, objective, zoom, exposure time and gain parameters) were constant throughout the imaging of all samples. A minimal threshold was set on appropriate control sections for each experiment in which only the light passing through the orthogonally oriented polarizers representing fibrous structures only (ie. excluding residual light from the black background) was included. The threshold was maintained for all images across all conditions within each experiment. The area of the transferred regions that was covered by the minimal thresholded light was calculated and a minimum 10 sections/vessels per condition were averaged together (Image J software).

Immunohistochemistry of human, rat and mouse lung: Lung sections (5 um) were deparaffinized and high temperature antigen retrieval was performed followed by blocking in TBS/BSA 5%, 10% goat serum and exposure to primary antibody and biotinylated secondary antibody (Vectastain ABC kit, Vector Labs). A primary antibody against YAP1 (1/200) was obtained from Cell Signaling. Primay antibody against TIMP2 (1/25), LRP8 (1/100) α-SMA (1/400) were purchased from Abcam. A primary antibody against MMP2 (1/250) was purchased from Milipore. A primary antibody against OCT4 (1/50) was purchased from Santa Cruz. In most cases, color development was achieved by adding streptavidin-biotinylated alkaline phosphatase complex (Vector Labs) followed by Vector Red alkaline phosphatase substrate solution (Vector Labs). Levamisole was added to block endogenous alkaline phosphatase activity (Vector Labs). Pictures were obtained using an Olympus Bx51 microscope. Small pulmonary vessels (<100 μm diameter) present in a given tissue section (>10 vessels per section) that were not associated with bronchial airways were selected for analysis (N>5 animals per group). Intensity of staining was quantified using ImageJ software (NIH). Degree of pulmonary arteriolar muscularization was assessed in paraffin-embedded lung sections stained for α-SMA by calculation of the proportion of fully and partially muscularized peripheral (<100 μm diameter) pulmonary arterioles to total peripheral pulmonary arterioles, as previously described (7). Medial thickness was also measured in α-SMA stained vessels (<100 μm diameter) using ImageJ software (NIH) and expressed as arbitrary units. All measurements were performed blinded to condition.

### Animals

Pulmonary Hypertension Models:

Eight-week-old littermate mice (C57Bl6) were injected—or not—with SU5416 (20 mg/kg; Sigma-Aldrich), followed by exposure to normobaric hypoxia (10% O2; OxyCycler chamber, Biospherix Ltd, Redfield, N.Y.) or normoxia (21% O2) for 3 weeks, right heart catheterization was performed followed by harvest of lung tissue for RNA extraction or paraffin embedding. Male Sprague-Dawley rats (10-14 week old) were injected with 60 mg/kg monocrotaline at time 0; at 0-4 weeks post-exposure, right heart catheterization was performed followed by harvest of lung tissue for RNA extraction or paraffin embedding, as described below (section: Tissue harvest).

Male Sprague-Dawley rats (10-14 week old) were injected with SU5416 (20 mg/kg; Sigma-Aldrich), followed by exposure to normobaric hypoxia (10% O2; OxyCycler chamber, Biospherix Ltd, Redfield, N.Y.) or normoxia (21% O2) for 3 weeks, right heart catheterization was performed followed by harvest of lung tissue for RNA extraction or paraffin embedding,

Lung Fibrosis Model:

C57BL/6 mice (7-8 weeks old) were exposed to 0.035 U of bleomycin via oropharyngeal route. In the control group, saline (PBS) was administered via the same route. Mice were killed 14 days or 21 days after bleomycin administration.

Forced expression of miR-130a in mouse lung: Eight-week-old mice (C57Bl6) were injected with SU5416 (20 mg/kg; Sigma-Aldrich), followed by 4 intrapharyngeal injections (once by week) of 1 nmol of miR-control (pre-miR-NC) or miR-130a (pre-miR-130a) mixed in 100 uL PBS solution containing 5% Lipofectamine 2000 (Life Technologies). Three days after the last injection, right heart catheterization was performed as previously described (12), followed by harvest of lung tissue for RNA extraction or paraffin embedding, as described above. BAPN (Sigma-Aldrich) was dissolved in water (30 mg/kg/day).

Inhibition of the miR-130/301 family in mouse model of PH: Eight-week-old mice (C57Bl6) were injected with SU5416 (20 mg/kg; Sigma-Aldrich), followed by exposure to normobaric hypoxia (10% O2; OxyCycler chamber, Biospherix Ltd, Redfield, N.Y.) for 2 weeks. After two weeks and confirmation of PH development in five mice (right heart catheterization), mice were further treated with 3 intrapharyngeal injections (every 4 days) of control or miR-130/301 shortmer oligonucleotides, designed as fully modified antisense oligonucleotides complementary to the seed sequence of the miR-130/301 miRNA family (10 mg/kg; Regulus). Specifically, the control and miR-130/301 shortmer oligonucleotides were non-toxic, lipid-permeable, high-affinity oligonucleotides. The miR-130/301 shortmer carried a sequence complementary to the active site of the miR-130/301 miRNA family, containing a phosphorothioate backbone and modifications (fluoro, methoxyethyl, and bicyclic sugar) at the sugar 2′ position. Three day after the last injection, right heart catheterization was performed followed by harvest of lung tissue for RNA extraction or paraffin embedding, as described above.

Inhibition of the miR-130/301 family in rat lung: Male Sprague-Dawley rats (10-14 week old) were injected with 60 mg/kg monocrotaline at time 0 followed by 5 intraperitoneal injections (every 3 days) of control or miR-130/301 shortmer oligonucleotides (20 mg/kg; Regulus). Three day after the last injection, right heart catheterization was performed followed by harvest of lung tissue for RNA extraction or paraffin embedding, as described above.

Inhibition of the miR-130/301 family in mouse model of pulmonary fibrosis.: Eight-week-old mice (C57Bl6) were injected with bleomycin (1.5 U/kg Sigma Aldrich) followed by 10 intraperitoneal injections (every 2 days) of control or miR-130/301 shortmer oligonucleotides (20 mg/kg; Regulus). Two day after the last injection lung tissue was harvest for RNA extraction or paraffin embedding.

Statistics: Cell culture experiments were performed at least three times and at least in triplicate for each replicate. The number of animals in each group was calculated to measure at least a 20% difference between the means of experimental and control groups with a power of 80% and standard deviation of 10%. The number of unique patient samples for this study was determined primarily by clinical availability. In situ expression/histologic analyses of both mouse and human tissue, and pulmonary vascular hemodynamics in mice were performed in a blinded fashion. Numerical quantifications for in vitro experiments using cultured cells or in situ quantifications of transcript/miRNA expression represent mean±standard deviation (SD). Numerical quantifications for physiologic experiments using mice or human reagents represent mean±standard error of the mean (SEM). Immunoblot images are representative of experiments that have been repeated at least three times. Micrographs are representative of experiments in each relevant cohort of mice. Paired samples were compared by Student's t test 2 tailed. A P value less than 0.05 was considered significant.

Studies approval.: ll animal experiments were approved by the Harvard Center for Comparative Medicine.

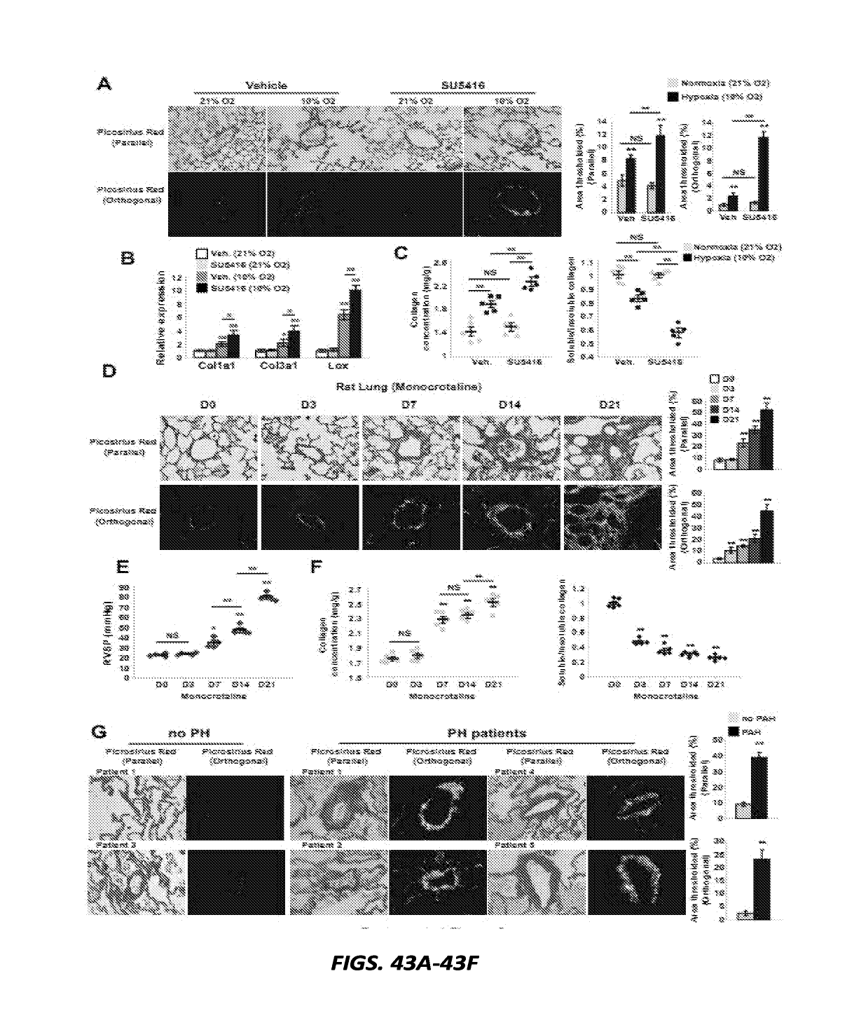

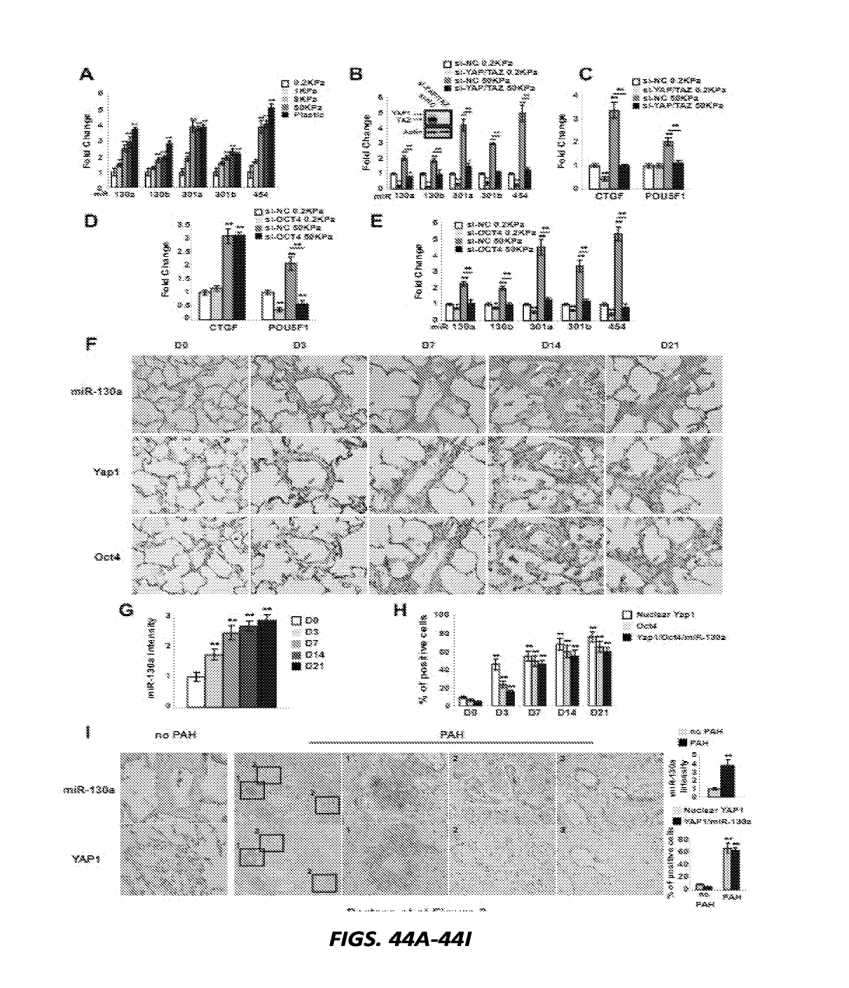

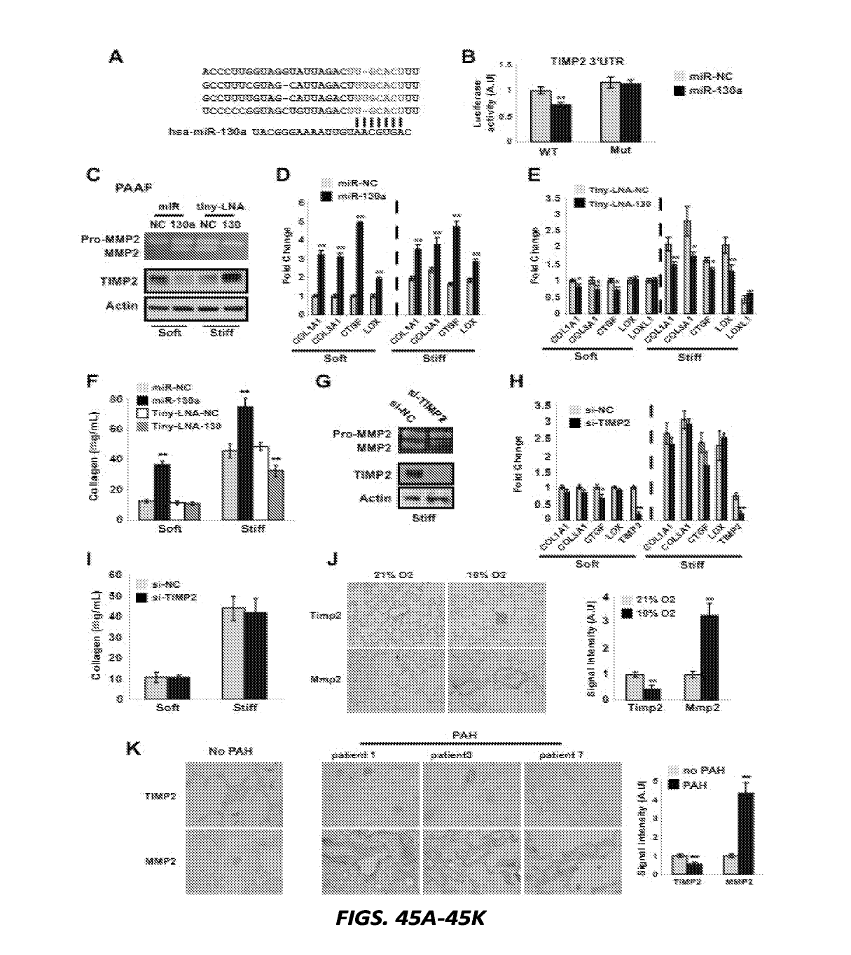

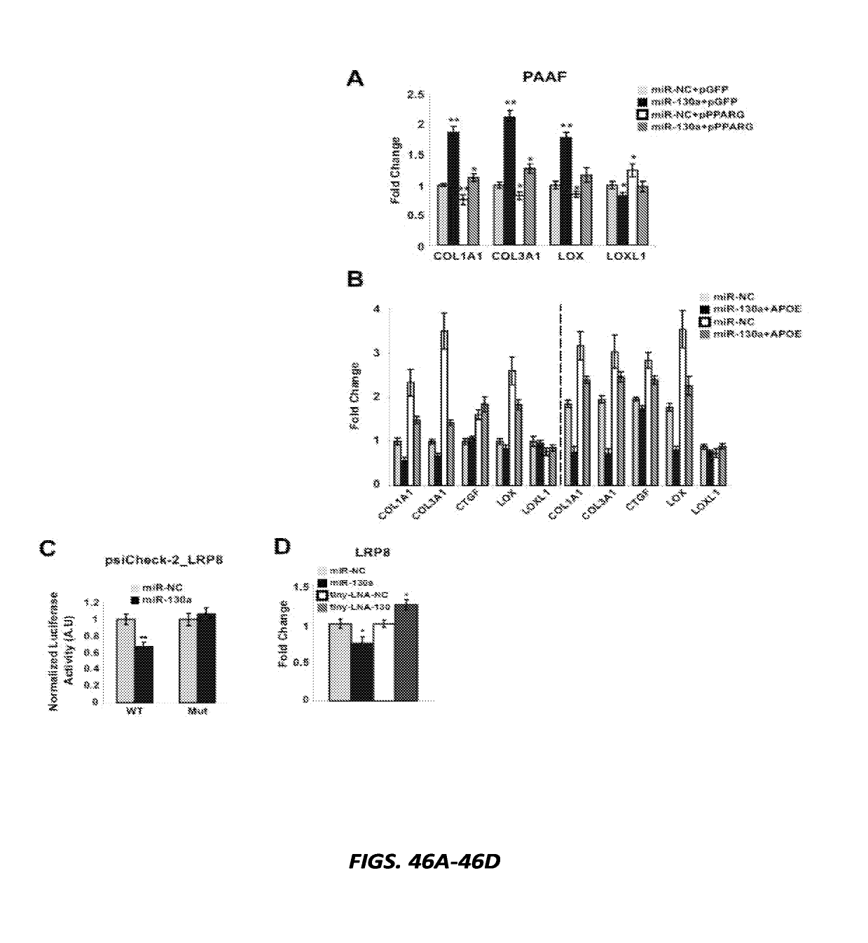

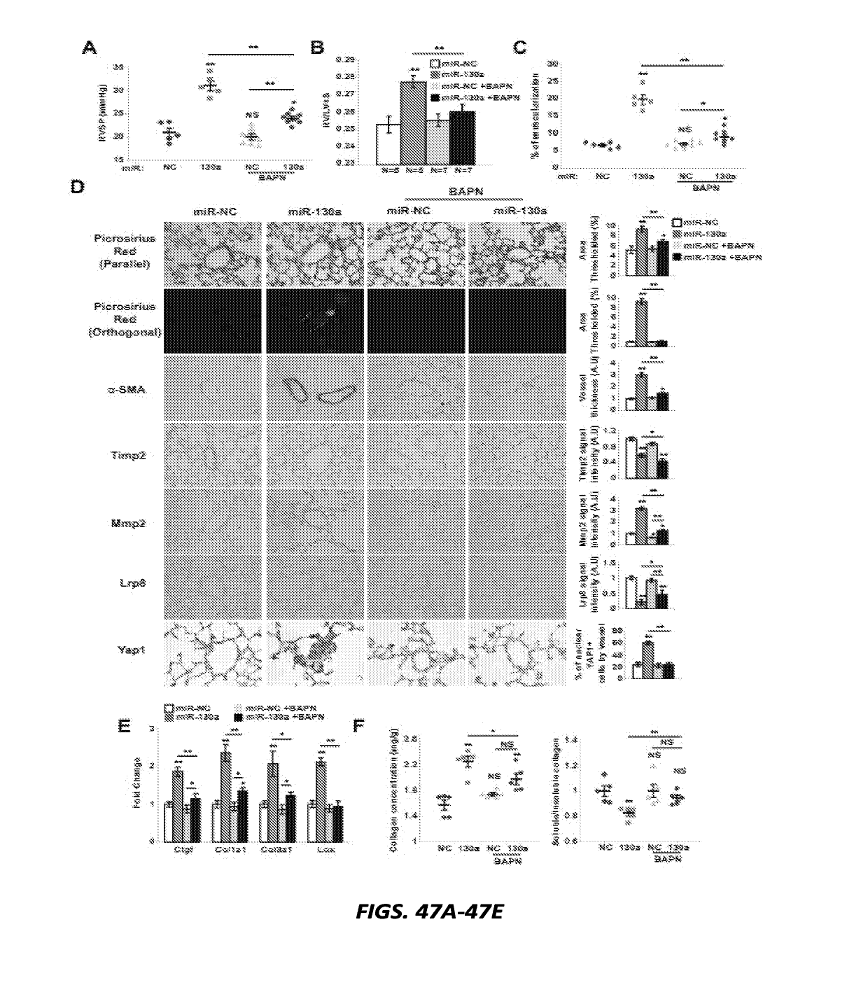

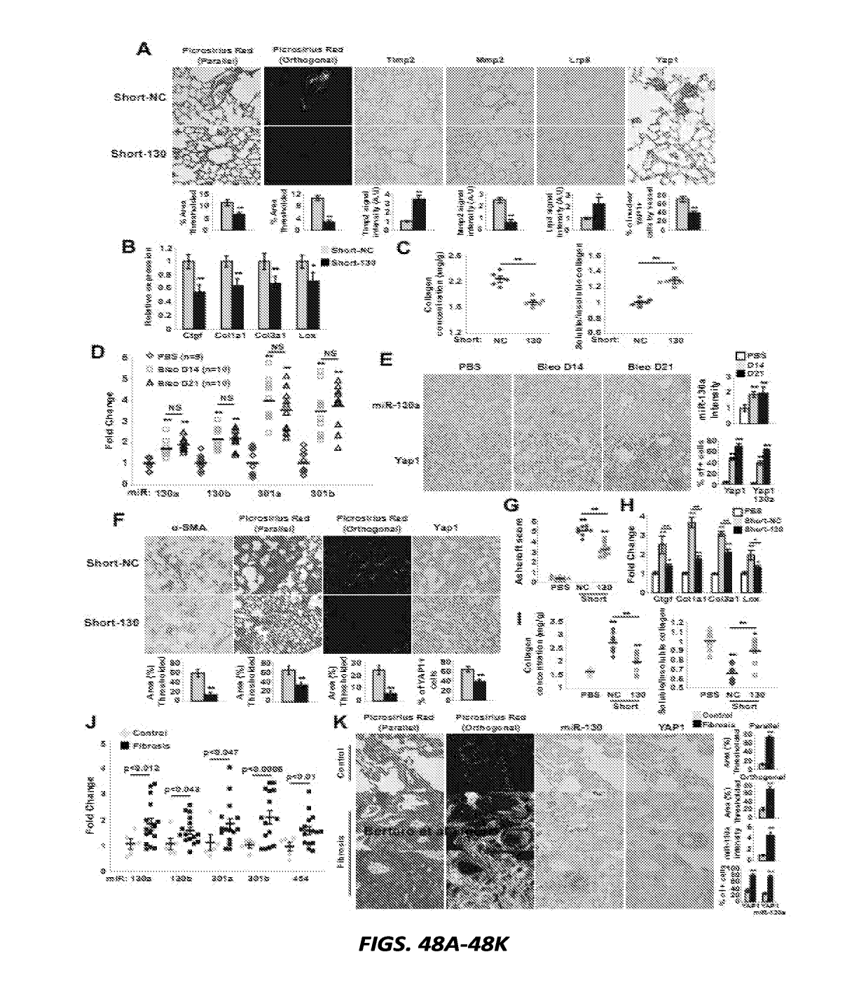

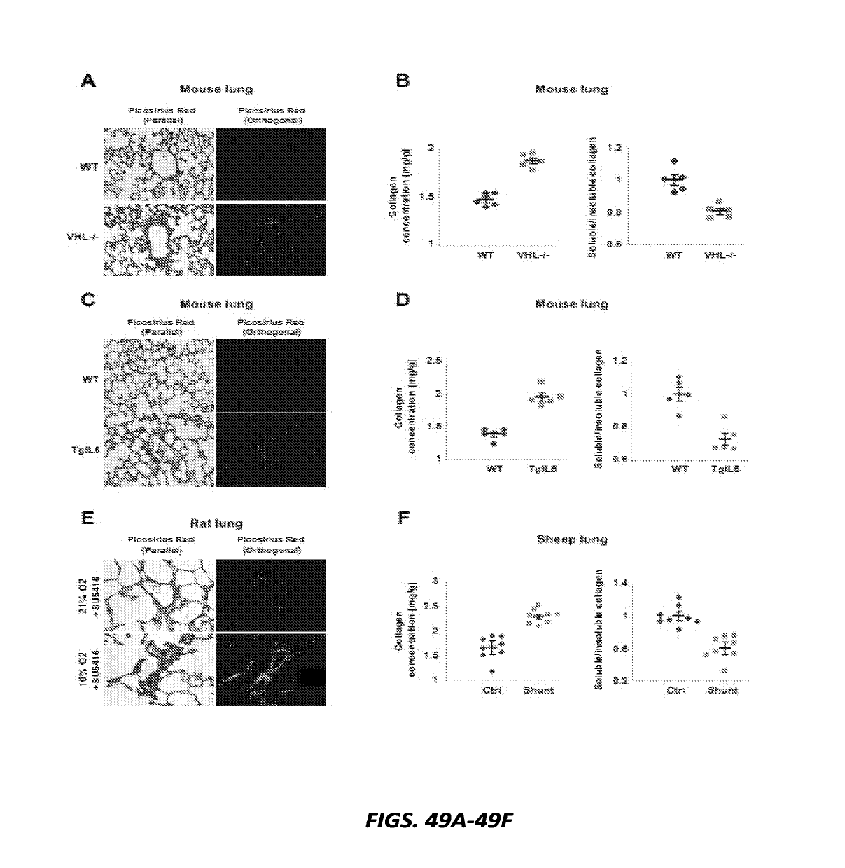

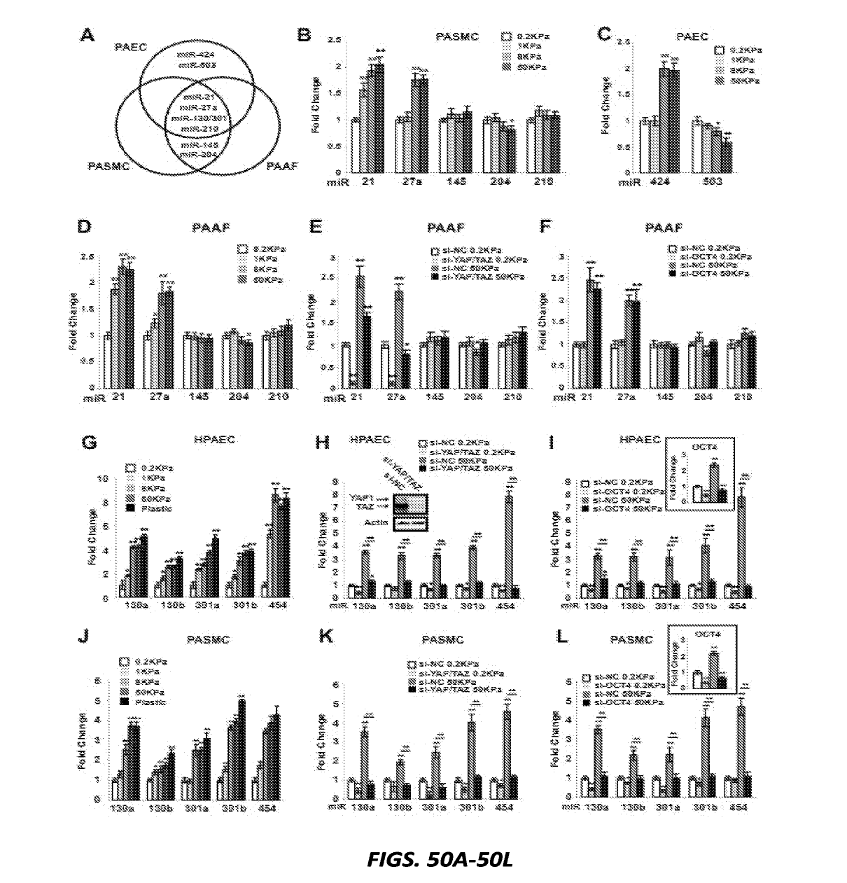

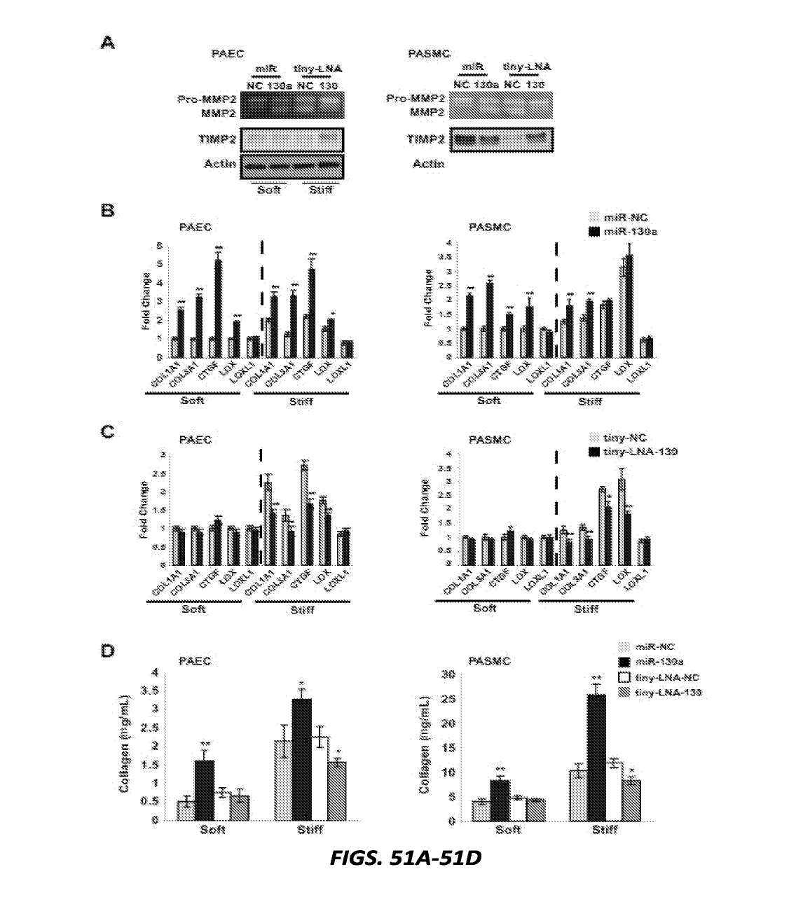

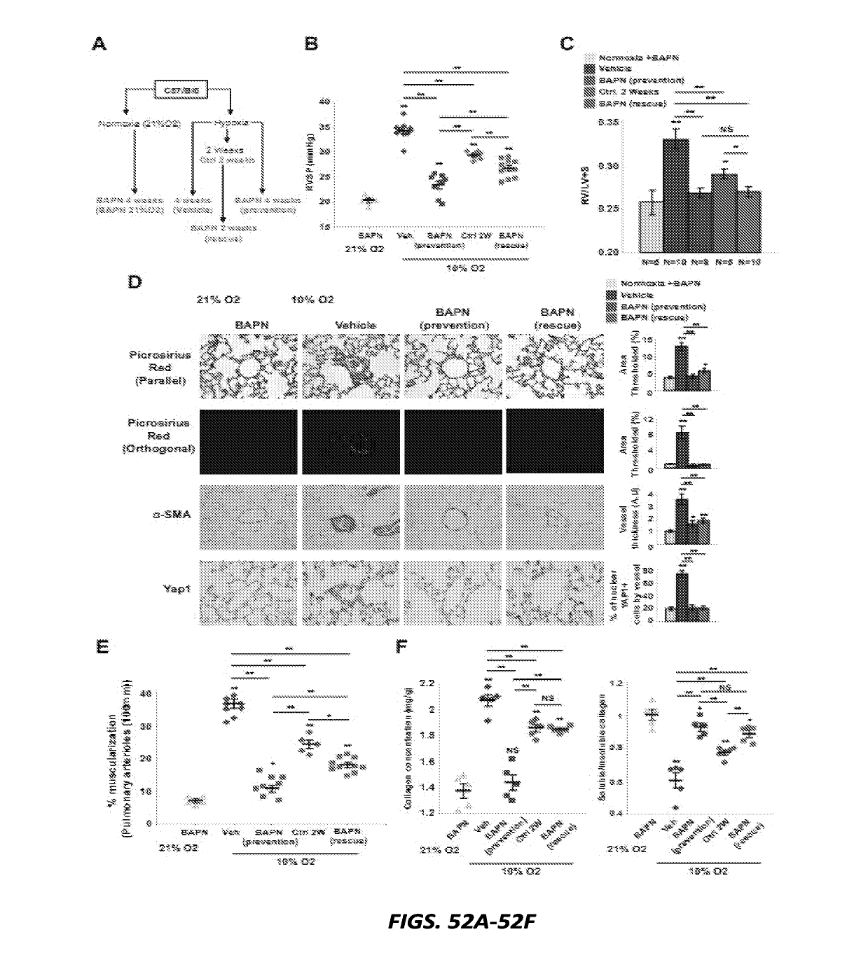

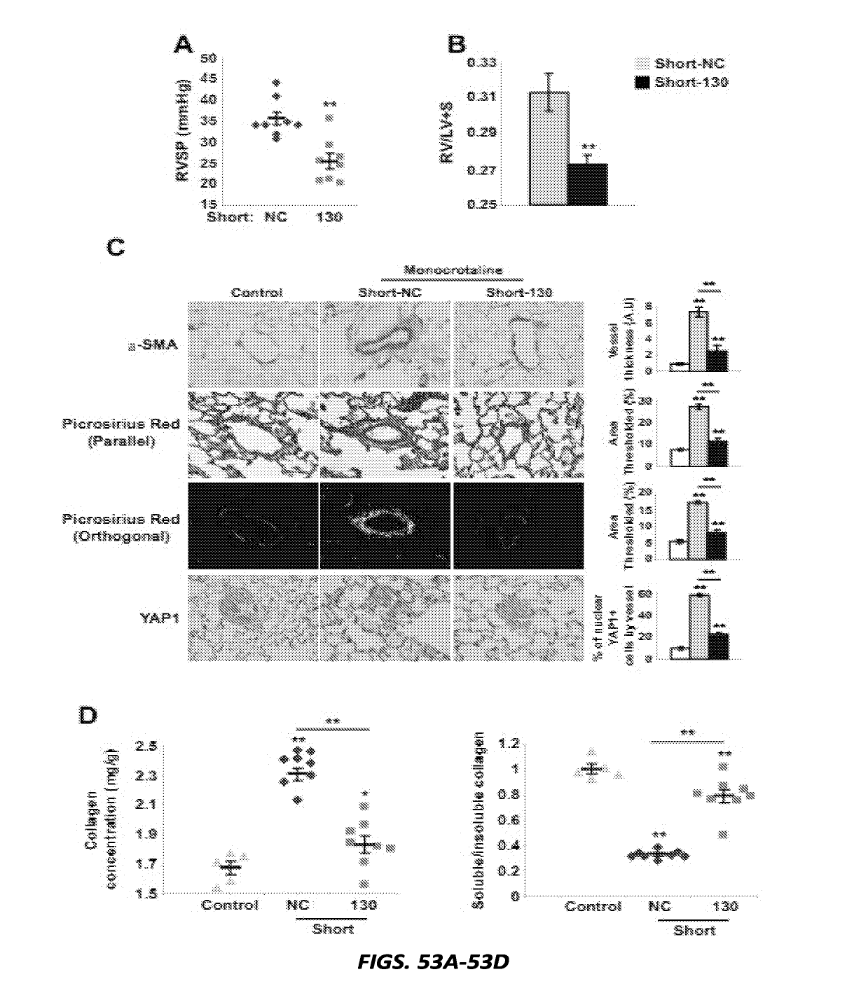

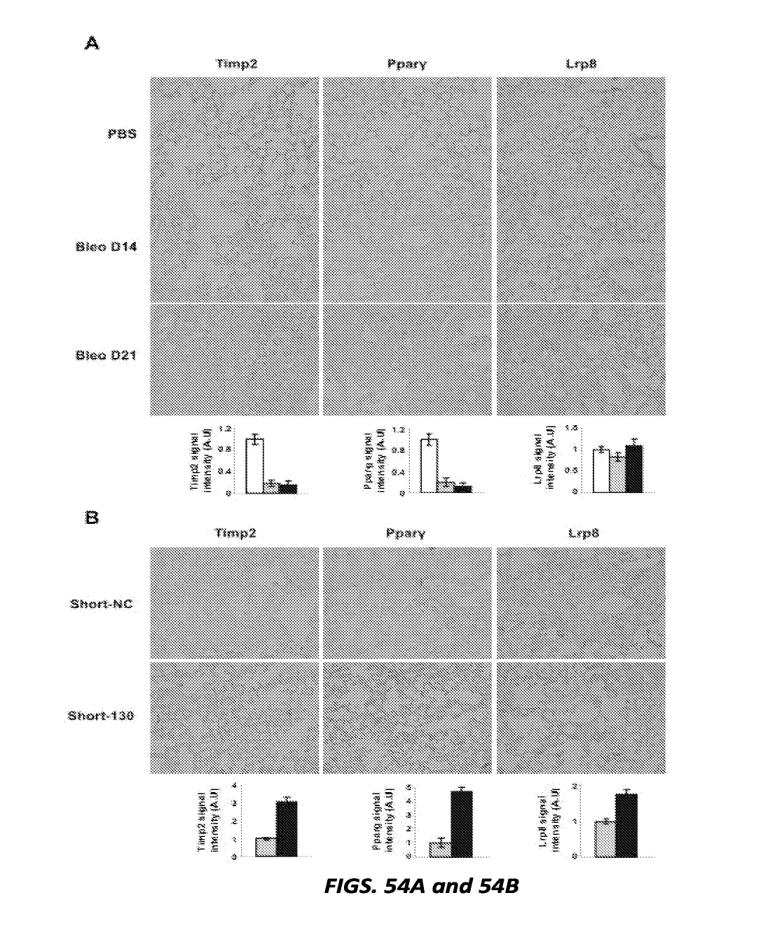

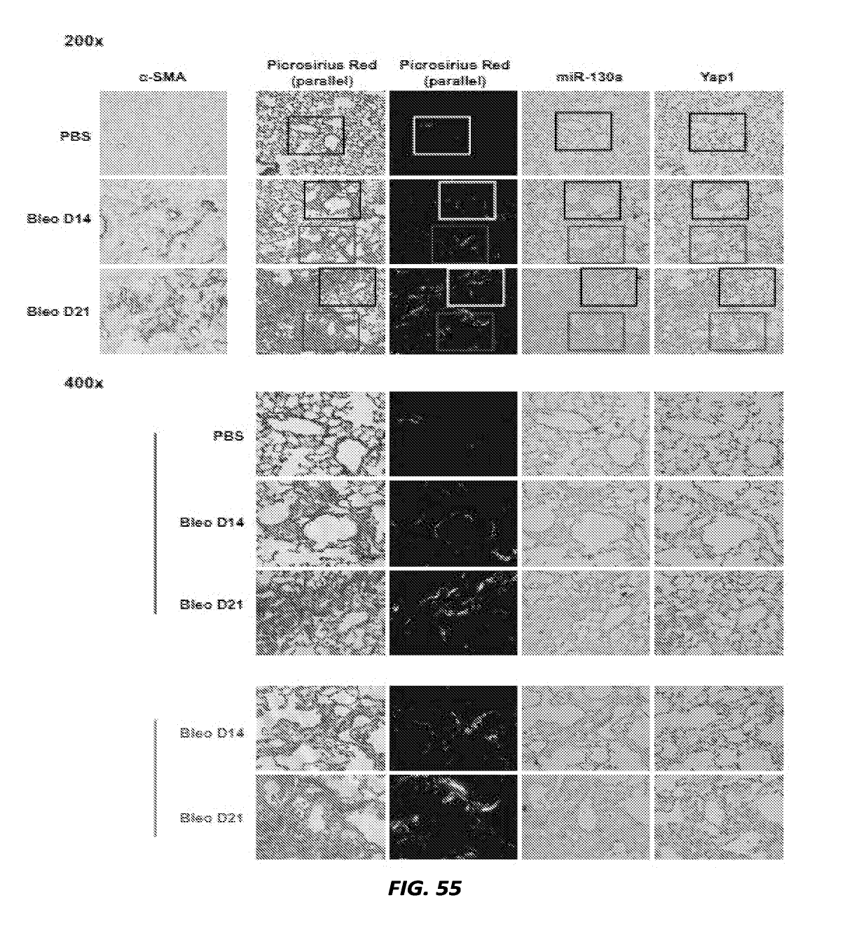

All experimental procedures involving the use of human tissue and plasma were approved by The Partners Healthcare and Boston Children's Hospital Institutional Review Boards and the New England Organ Bank. Ethical approval for this study conformed to the standards of the Declaration of Helsinki. Informed consent was obtained for right heart catheterization and blood sampling. For formalin-fixed paraffin-embedded lung samples, human PH specimens were collected from unused or discarded surgical samples; non-diseased human lung specimens from the New England Organ Bank have been described (62). Data is shown in FIGS. 43A-55.

### Example 5: Matrix Crosslinking Promotes Pulmonary Hypertension Through Mechanoactivation of the MicroRNA-130/301 Family

Pulmonary hypertension (PH) is a deadly but enigmatic vascular disease of increasing prevalence worldwide (Chan and Loscalzo, 2008a). PH can be induced by myriad triggers, and a growing number of molecular pathways, as well as pathogenic crosstalk between pulmonary artery endothelial cells (PAECs) and pulmonary artery smooth muscle cells (PASMCs), have been described to influence PH. A great majority of the molecular targets chosen for clinical testing primarily affect the hyperproliferative, vasoconstrictive, and pro-thrombotic phenotypes that predominate the end-stage of disease (Boutet et al., 2008; Stenmark and Rabinovitch, 2010). However, while contemporary therapies improve patient survival and quality of life (Apostolopoulou et al., 2007; Badesch et al., 2007; Galie et al., 2006; Gan et al., 2007a; McLaughlin et al., 2005), they fail to target the still enigmatic origins of PH and neither reverse or prevent disease.

Remodeling of the extracellular matrix (ECM) may be an upstream cause of PH that has only recently been explored. Vascular stiffness in the proximal and distal pulmonary arterial tree occurs in various forms of PH (Lammers et al., 2012; Wang and Chesler, 2011), and such stiffness is an index of disease progression (Gan et al., 2007b; Mahapatra et al., 2006). Yet, additional molecular insights are only just emerging. In both health (Schafer and Werner, 2008) and disease (Butcher et al., 2009) in tissue contexts beyond PH, ECM remodeling is a complex process, occurring through changes in the balance between matrix (e.g., collagen and elastin) deposition and matrix degradation, and through collagen crosslinking enzymes such as lysyl oxidase (LOX). In that context, two related transcriptional coactivators, YAP (Yes Associated Protein 1) and TAZ (Transcriptional Coactivator with PDZ-Binding Motif), are crucial for mechanotransduction, a process that converts extracellular mechanical cues into intracellular signaling (Dupont et al., 2011) and regulates cellular proliferation, and survival (Cordenonsi et al., 2011; Harvey et al., 2013), ECM remodeling (Fujii et al., 2012; Liu et al., 2015; Tang et al., 2013) as well as organ growth (Pan, 2010). However, two undefined concepts remain: 1) the pathways of mechanotransduction that regulate ECM remodeling upstream and downstream of YAP/TAZ; and 2) their exact relation to PH.

MicroRNAs (miRNAs) are essential mediators of multiple cellular processes involving cell-cell and cell-matrix interactions (Valastyan and Weinberg, 2011). Individual miRNAs often control multiple target genes and phenotypes, making them attractive candidates as upstream “master” regulators of seemingly diverse processes. Crosstalk between miRNA biology and the biomechanical ECM properties, however, has been largely unexplored. Here, we found that peri-arteriolar ECM modification is an early event of PH progression in vivo, driven by biomechanical induction in fibroblasts of a central regulatory circuit including the microRNA-130/301 (miR-130/301) family and YAP/TAZ. Recently, we described the pro-proliferative actions of miR-130/301 in PH (Bertero et al., 2014b). Yet, beyond proliferation, we now found that the miR-130/301 family engages a network of related gene targets including PPARγ-APOE-LRP8 axis and LOX activity to coordinate ECM remodeling and sustain stiffening. Emphasizing the relevance of ECM remodeling in hereditary PH, factors genetically associated with PH were found to communicate with this YAP/TAZ-miR-130/301 feedback loop. Finally, in vivo pharmacologic inhibition of microRNA-130/301, APOE, or LOX activity prevented and reversed this fibrotic pathophenotype and PH. Consequently, we present a new therapeutic concept in PH, focused on reduction of microRNA-dependent ECM stiffening and tailored manipulation of the miR-130/301-YAP/TAZ feedback circuit.

### Results

PH is Characterized by a Programmatic Shift in Fibrotic Gene Expression and Early Initiation of Peri-Arteriolar Collagen Remodeling:

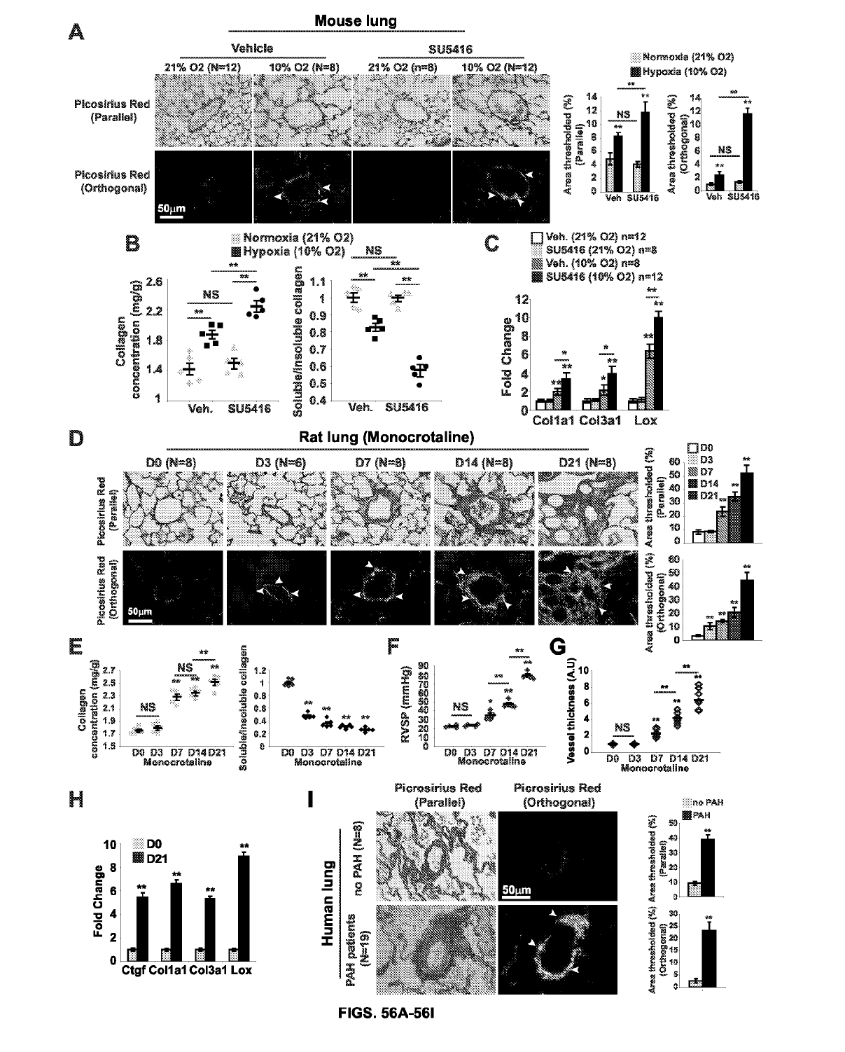

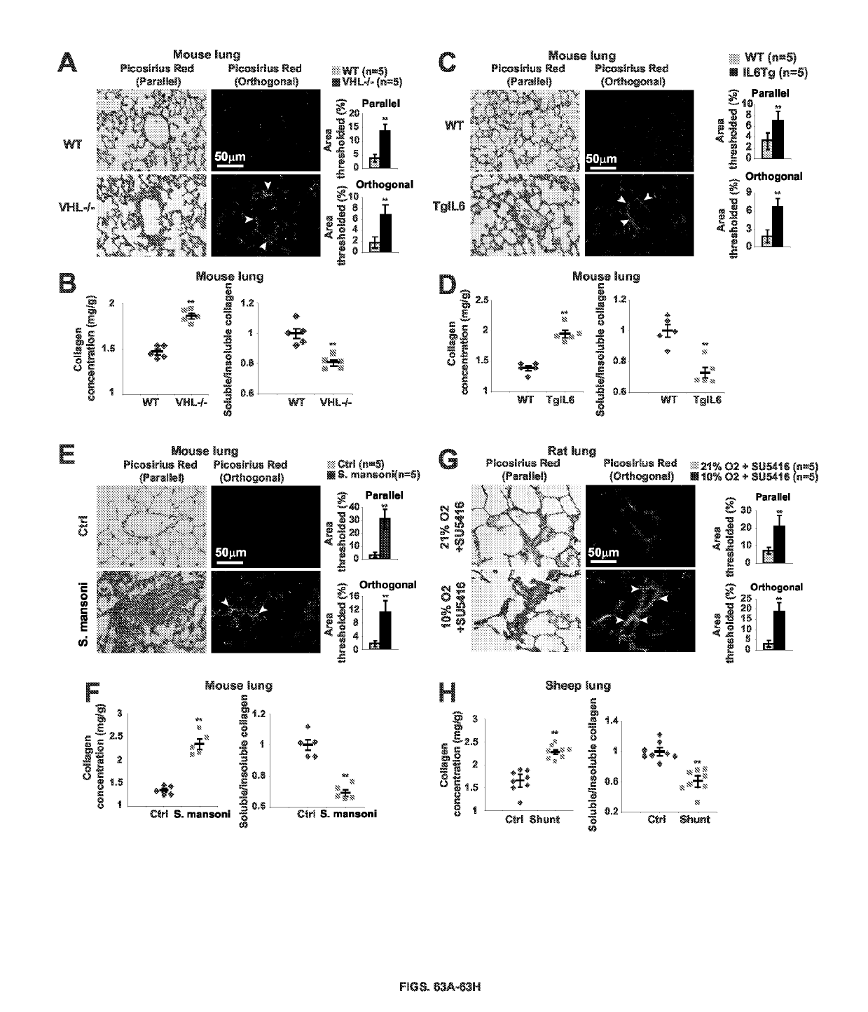

To characterize initially the relevance of fibrosis in PH, we performed a transcriptomic analysis of PH lung tissue from mice (chronic hypoxia with administration of the VEGF receptor antagonist SU5416) (Ciuclan et al., 2011). RNA-sequencing coupled with pathway enrichment revealed dysregulated genes involved in ECM plasticity specifically (index pathway #4 ECM organization, #7 ECM receptor interaction; Table 8) as well as pathways linked to ECM stiffening and the collagen crosslinking enzyme LOX (index #1 and #18; Table 8). We also analyzed eight different animal models of PH—including hypoxia-driven models (mice exposed to chronic hypoxia alone, mice and rats exposed to chronic hypoxia+SU5416 (FIGS. 56A-C, FIGS. 63A-63H), and VHL-null mice (FIGS. 63A-63H); inflammatory-driven models (mice expressing transgenic IL-6 (FIGS. 63A-63H), monocrotaline-exposed rats (FIGS. 56D-H), and Schistosoma mansoni-infected mice (FIGS. 63A-63H); and a surgical lamb model of congenital heart disease (FIG. 63). In each model, pulmonary collagen content and crosslinked fractions were increased, as assessed by biochemical analysis of pulmonary tissue and in situ Picrosirius Red stain. Moreover, in monocrotaline-induced PH in rats, the early phases of PH development (3 days post-monocrotaline exposure) were characterized by increased peri-arteriolar collagen crosslinking (FIG. 56D-E) prior to hemodynamic PH manifestation (as assessed by right ventricular systolic pressure, RVSP) (FIG. 56F) or increase of medial thickening (FIG. 56G). Similar perivascular fibrosis was observed in human PAH tissue [cohorts described in (Bertero et al., 2014b), FIG. 56I]. Together, these results demonstrate that peri-arteriolar ECM remodeling is an early process in a number of PH subtypes and is associated with a programmatic shift in a network of ECM-related genes.

ECM Stiffness is a Mechanical Stimulus for miR-130/301 Family Expression Via YAP/TAZ Signaling.

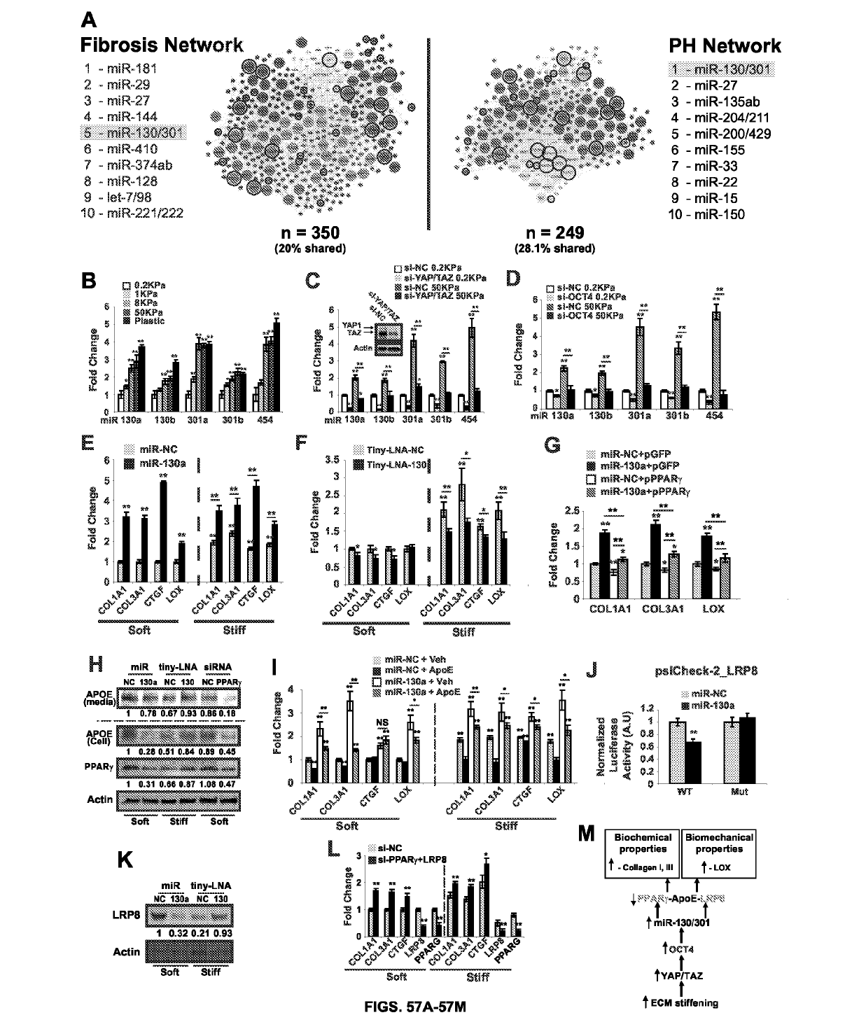

Previously, we found that miR-130/301 family members (miR-130a/b; miR-301a/b, and miR-454), which share the same putative gene target pool, promote cellular proliferation (Bertero et al., 2014b). Yet, based on the number and architectural distribution of predicted miRNA targets within a network of PH-relevant genes (“miRNA spanning score” as in Experimental Procedures), these miRNAs were predicted to exert functions that best span the entire extent of these PH-relevant genes (Bertero et al., 2014b). Among the predicted pool of miR-130/301 target genes, a broader component of factors related ECM remodeling was evident (FIG. 57A, encircled genes). Thus, to delineate further the connections among this miRNA family, the ECM, and PH, we constructed in silico a “fibrosis network” based on curated seed genes known to be causatively involved in ECM remodeling (Table 9) and their first degree interactors (Table 10, see Experimental Procedures). The final “fibrosis network” included 350 genes and 1459 interactions (FIG. 57A, Table 10) and displayed substantial identity (FIG. 57A, encircled and enlarged genes) with a previously described PH network (Bertero et al., 2014b) constructed from a similarly curated set of PH-specific disease genes and their closest first-degree interactors. Specifically, 70 members of the fibrosis network appeared in the PH network (28.3% of PH nodes). Furthermore, miR-130/301 members were highly-ranked by spanning score (Rank #5 among all conserved miRNAs in TargetScan 6.2) indicating their control over multiple pathways in the fibrosis network (FIG. 57A, Table 11). Thus, as predicted by network analysis, the miR-130/301 family carries both overlapping and systems-wide effects in both the PH and fibrosis networks.

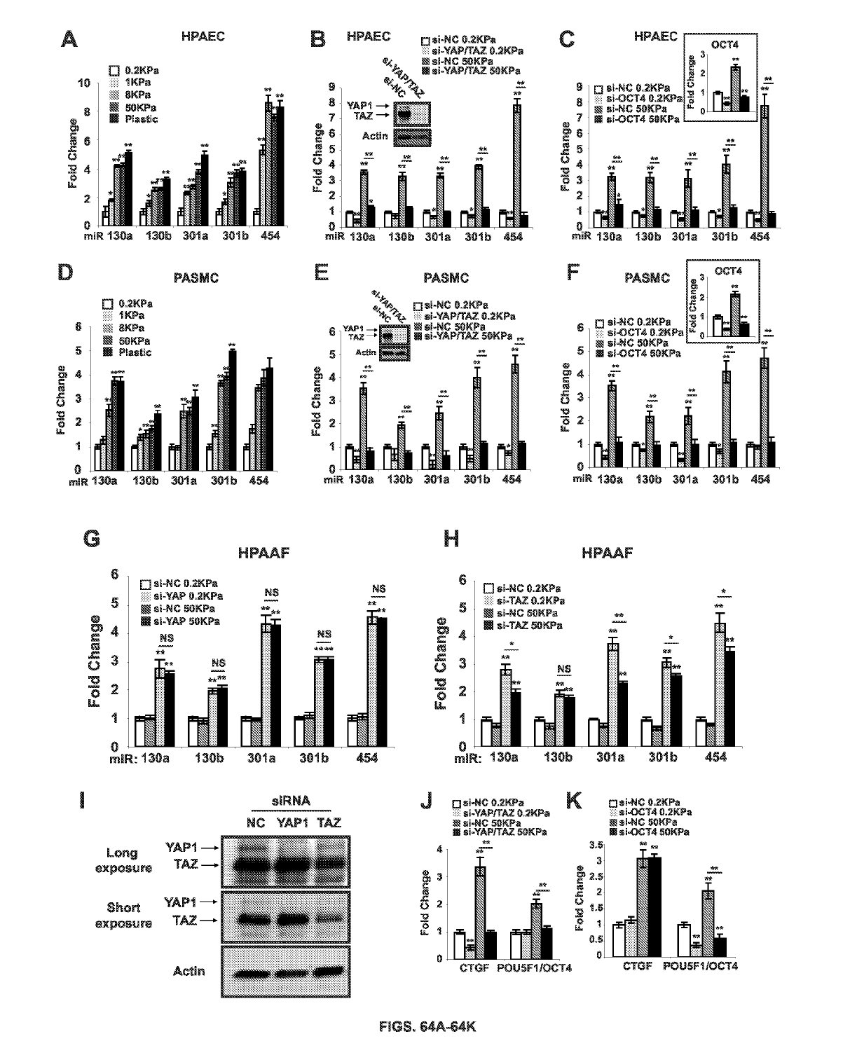

To begin to interrogate experimentally the connection of miR-130/301 to the ECM, we found that miR-130/301 members were up-regulated in pulmonary arterial adventitial fibroblasts (PAAFs) after culture in hydrogels of increasing stiffness (FIG. 2B). Only siRNA knockdown of the mechanosensitive factors YAP and TAZ together (FIG. 57C), but not separately (FIG. 64), reversed this up-regulation, indicating the dependence of miR-130/301 induction upon coordinated activation of YAP/TAZ. No YAP/TAZ-specific TEAD binding site was predicted in the promoter regions of these miRNAs. Instead, we postulated that YAP/TAZ may induce miR-130/301 via increase of the transcription factor POU5F1/OCT4, a target of YAP/TAZ (Lian et al., 2010) and a factor that up-regulates miR-130/301 in hypoxia (Bertero et al., 2014b). Indeed, POU5F1/OCT4 was induced by matrix stiffness in PAAFs and reversed by YAP/TAZ knockdown (FIGS. 64A-64K), and knockdown of POU5F1/OCT4 (FIGS. 64A-64K) reversed miR-130/301 mechanoinduction (FIG. 57D). Similar findings were observed in other pulmonary vascular cell types (FIGS. 64A-64K). Together, these results indicate that, across cell types, coordinated up-regulation of the miR-130/301 family by mechanical ECM stiffening is mediated by a unique YAP/TAZ- and POU5F1/OCT4-dependent pathway.

The miR-130/301 Family Controls a Cohort of Factors Relevant to Fibrosis in the Lung.

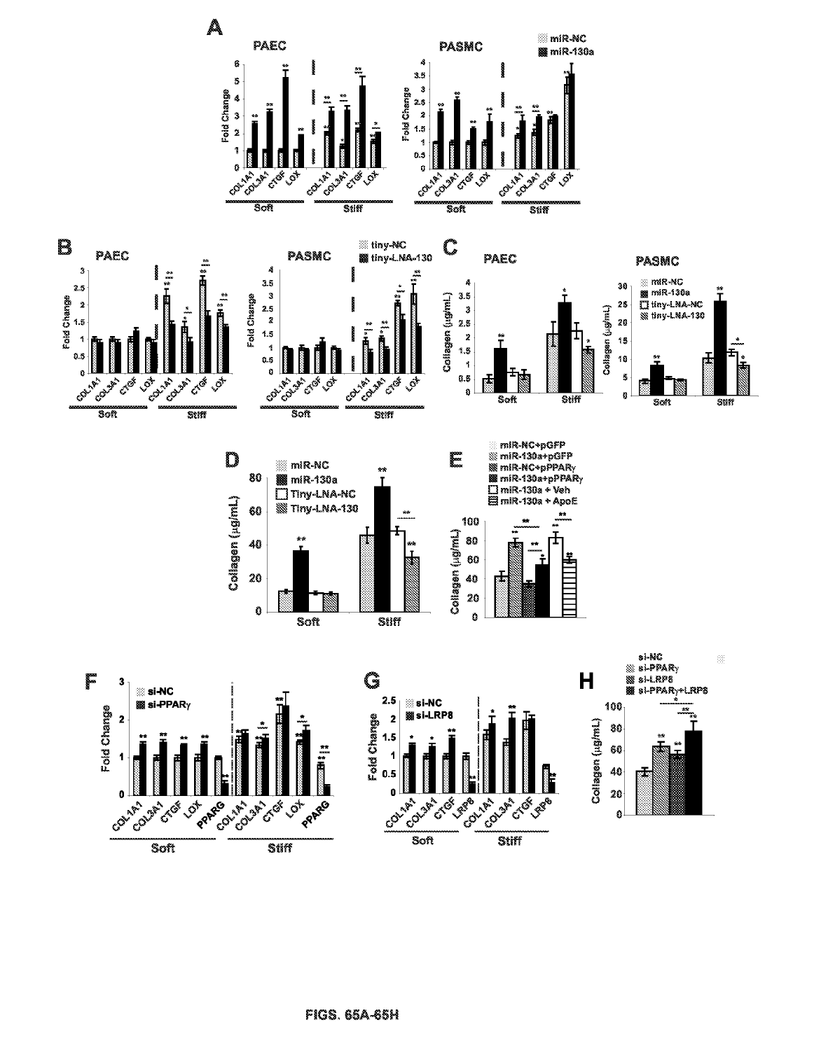

Downstream of YAP/TAZ, we examined the consequent effects of miR-130/301 on known fibrotic factors within the lung as well as biochemical properties of the ECM. In cultured PAAFs, either forced miR-130a expression or high ECM stiffness increased transcripts encoding collagen isoforms, LOX, and CTGF, a marker of ECM stiffening and fibrosis (FIG. 57E), as well as increased secretion of collagen (FIGS. 65A-65G). Conversely, in high ECM stiffness, short locked nucleic acid inhibitory oligonucleotides (“tiny-LNAs”) with antisense complementarity to the miR-130/301 seed sequence (tiny-LNA-130) decreased transcript expression of this fibrotic gene cohort (FIG. 57F) and decreased collagen secretion (FIGS. 65A-65G). Thus, these results define miR-130/301 as a broad molecular regulator of ECM modification.

miR-130/301 Controls Collagen Deposition and Crosslinking Via the PPARγ-APOE-LRP8 Axis.

To elucidate molecular factors facilitating collagen deposition and crosslinking downstream of miR-130/301, the peroxisome proliferator-activated receptor gamma (PPARγ) was studied—a known target of miR-130/301 in control of cellular proliferation (Bertero et al., 2014b), with a known role in ECM modification in other contexts (Wei et al., 2012). Constitutive PPARγ disrupted the miR-130/301-mediated up-regulation of collagen and LOX in PAAFs (FIG. 57G). Notably, miR-130a overexpression or PPARγ knockdown (FIG. 57H) decreased apolipoprotein E (ApoE), a direct target of PPARγ (Hansmann et al., 2008) involved in ECM remodeling (Kothapalli et al., 2012). Conversely, miR-130/301 inhibition in stiff ECM increased ApoE (FIG. 57H). Furthermore, PAAFs exposed to media enriched with ApoE were similarly resistant to miR-130/301 activity (FIG. 57I). Constitutive PPARγ (pPPARγ) or ApoE treatment disrupted the miR-130/301-mediated up-regulation of secreted collagen (FIG. 65), indicating that miR-130/301 critically relies on PPARγ and ApoE to control collagen deposition and remodeling.

Given the role of ApoE and a predicted miR-130/301 binding site in the apolipoprotein E receptor LRP8 transcript [TargetScan 6.2 (Friedman et al., 2009)], we considered that LRP8 may also function prominently here. Via standard luciferase assay, we confirmed LRP8 as a direct miR-130/301 target (FIG. 57J). In cultured PAAFs, miR-130a decreased LRP8 expression, while inhibition of miR-130/301 increased LRP8 (FIG. 57K). As with PPARγ knockdown, LRP8 knockdown increased collagen expression (FIGS. 65A-65G). Importantly, knockdown of PPARγ and LRP8 together induced a more robust response than either knockdown alone, indicating their coordinated roles in controlling collagen expression (FIG. 57L and FIGS. 65A-65G). Similar findings were observed in other pulmonary vascular cell types (FIGS. 65A and 65B)

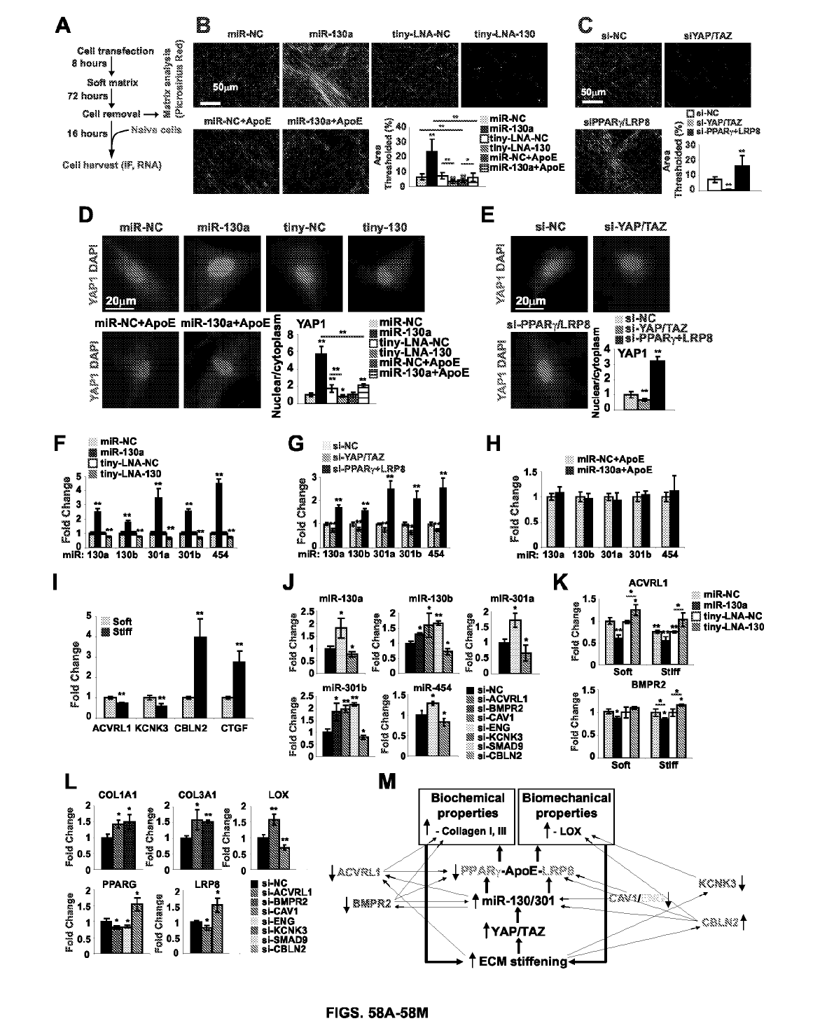

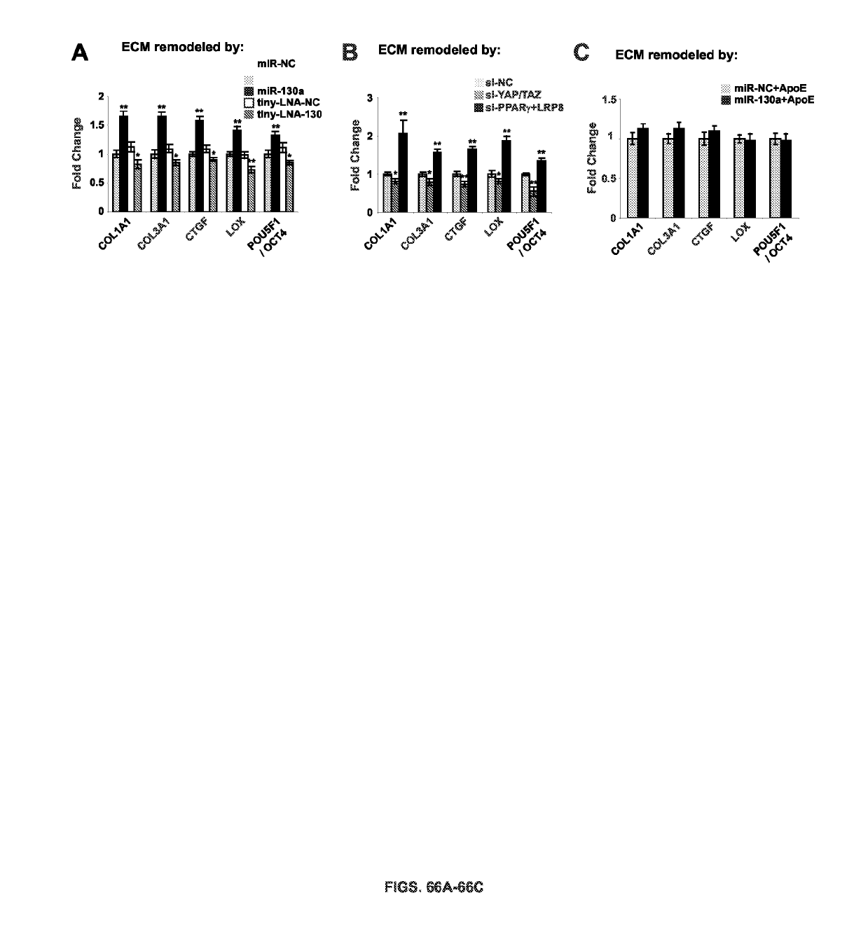

Finally, we postulated that miR-130/301 may be central to a feedback loop amplifying fibroblast activation and ECM stiffening. PAAFs were transfected with miR-130a mimic oligonucleotides or siRNAs for PPARγ and LRP8 and cultured in soft matrix to remodel the ECM. Cells were then removed from this ECM and replaced with non-transfected, naïve PAAFs (FIG. 58A; analyses of remodeled ECM in FIG. 58B-C). After 16 hours, ECM remodeling induced YAP nuclear localization (FIG. 58D-E) and increased POU5F1/OCT4 (FIGS. 66A and 66B), miR-130/301 (FIG. 58F-G), and downstream fibrosis-relevant genes (FIGS. 66A and 66B) in naive PAAFs. Conversely, ECM modification resulting from miR-130/301 inhibition or siRNA knockdown of YAP/TAZ decreased YAP nuclear localization (FIG. 58D-E), with a consequent reduction in POU5F1/OCT4, the fibrosis gene cohort (FIGS. 66A and 66B), and miR-130/301 (FIG. 58F-G). Moreover, the ECM modifications activating downstream fibrosis-relevant genes and miR-130/301 were reversed by ApoE (FIGS. 58B, 58D, 58H, 66A and 66B). Taken together, these results reveal that miR-130/301 and YAP/TAZ act in a feedback-driven, self-amplifying regulatory loop that integrates multiple direct target genes including PPARγ and LRP8 in order to regulate coordinately ECM remodeling and stiffening.

Factors Genetically Associated with PAH Exert Convergent Actions on the YAP/TAZ-miR-130/301 Circuit and ECM Plasticity.

We wanted to identify the originating triggers that activate the YAP/TAZ-miR-130/301 feedback circuit and thus initiate this fibrotic pathophenotype in PH. Disparate genes have been identified that predispose to hereditary PAH [such as ACVRL1, BMPR2, CAV1, ENG, SMAD9, as well as CBLN2 and KCNK3, as reviewed in (Austin and Loyd, 2014)], but together, these factors are not otherwise known to carry overlapping functions. We investigated if these genes may trigger this feedback loop and share a role in ECM plasticity. First, among these genes in PAAFs, ECM stiffening decreased ACVRL1 and KCNK3, factors with loss-of-function mutations in PAH (Chaumais et al., 2013; Harrison et al., 2003)), but increased CBLN2, a factor up-regulated in PAH kindreds (Germain et al., 2013) (FIG. 581). Second, siRNA-mediated knockdown of a separate subset of this gene cohort, ENG, CAV1 and BMPR2 (all with known loss-of-function mutations in PAH), induced miR-130/301 expression (FIG. 58J). Third, miR-130a in both stiff and soft ECM decreased ACVRL1 and BMPR2, a predicted direct target of miR-130/301 (Targetscan 6.2) (FIG. 58K). Conversely, miR-130/301 inhibition up-regulated ACVRL1 and BMPR2, restoring ACVRL1 in stiff matrix to its basal level. Finally, we found that knockdown of ACVRL1 and BMPR2 increased collagen; knockdown of KCNK3 increased LOX while knockdown of CBLN2 decreased LOX; and knockdown of ACVRL1, ENG, CAV1, and CBLN2 modulated the PPARγ-LRP8 axis (FIG. 58L). Thus, a network of upstream factors linked to hereditary PAH converges upon the YAP/TAZ-miR-130/301-PPARγ-LRP8 axis in order to remodel ECM (FIG. 58M). As such, these findings define the unique central importance of this ECM-relevant molecular circuitry among seemingly disparate genetic factors in PAH, thus solidifying the principle of miR-130/301-induced vascular stiffness as a primary driver of PAH in vivo.

miR-130/301 Reprograms Fibrotic Gene Expression to Increase Collagen Deposition and Collagen Crosslinking in PH In Vivo.

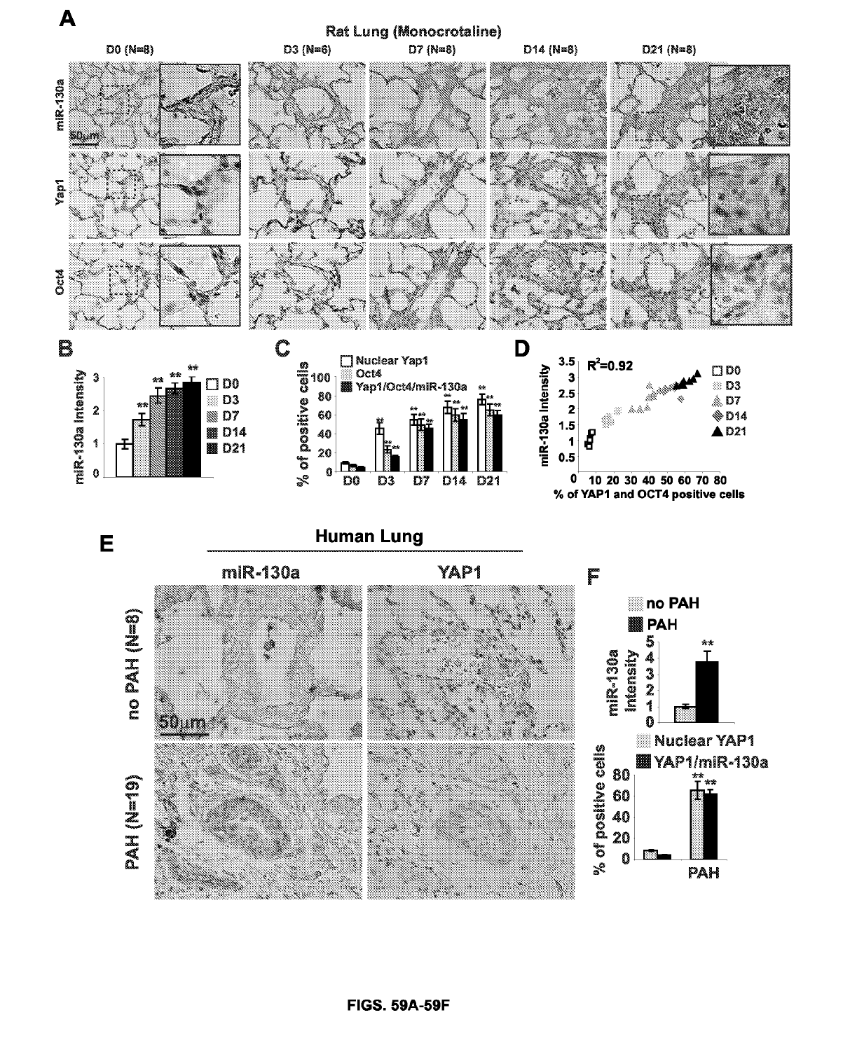

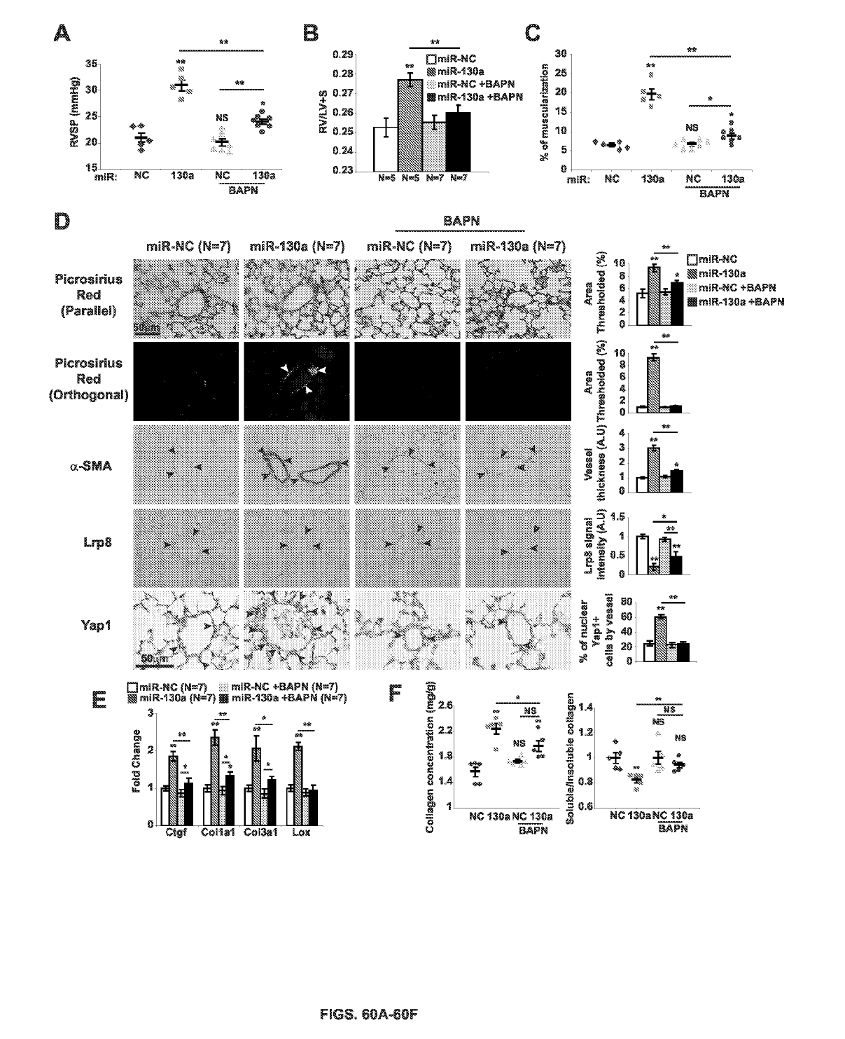

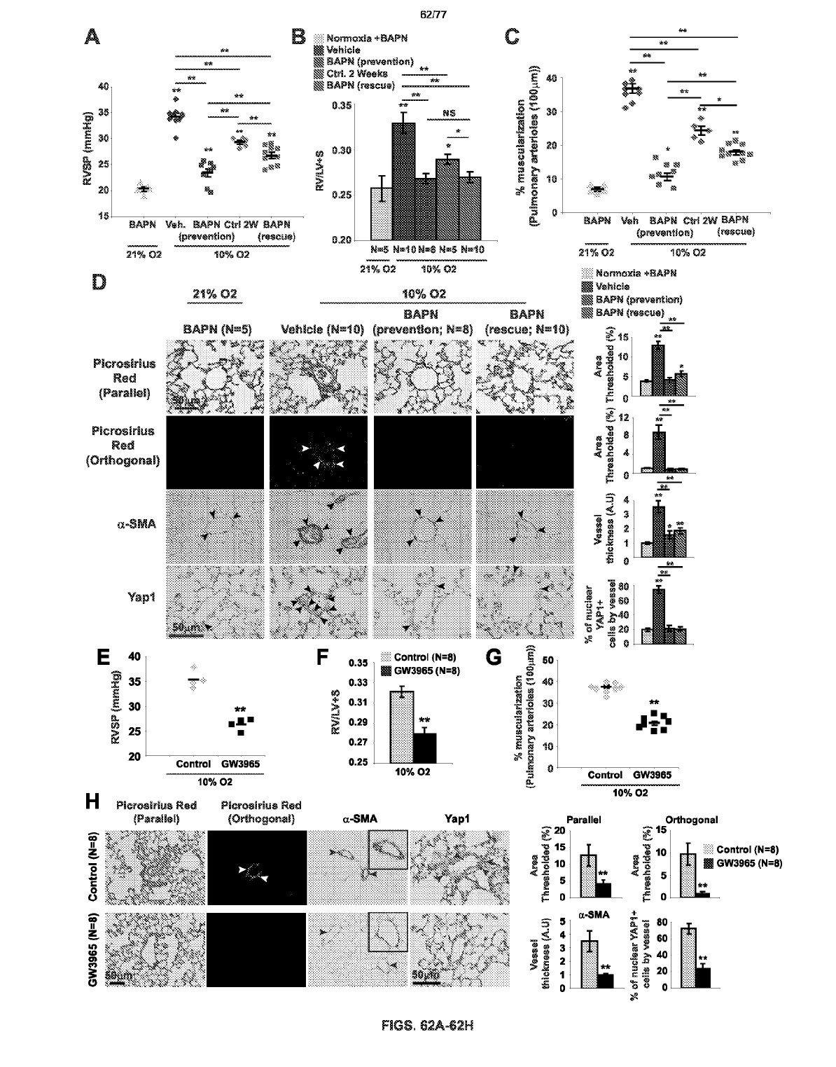

We wanted to determine whether the YAP/TAZ-miR-130/301 loop is active in PH across rodent models and human patients in vivo. In monocrotaline-treated rats with PH (FIG. 59A-D) and human patients [as described in (Bertero et al., 2014b)] (FIG. 59E-F), in situ peri-arteriolar staining displayed a strong positive correlation among YAP, POU5F1/OCT4, and miR-130a expression. To demonstrate the participation in vivo of miR-130/301 on collagen deposition and collagen crosslinking, miR-130a mimic oligonucleotides were delivered in mice via serial intrapharyngal delivery (FIG. 60A-C) to promote PH, as previously described (Bertero et al., 2014b). miR-130a-dependent PH was accompanied by vascular YAP nuclear localization, YAP activation (as reflected by Ctgf expression), and increased collagen deposition and crosslinking (FIG. 60D-F). Conversely, inhibition of the collagen crosslinking enzyme LOX by BAPN treatment (as in FIGS. 62A-62H) in miR-130-exposed mice reversed YAP activation as well as downstream PH manifestations (FIGS. 60A-60F) in a manner similar to its effects on hypoxia-induced PH (FIGS. 62A-62H). Thus, miR-130a is sufficient to promote LOX-dependent, vascular fibrosis in PH.

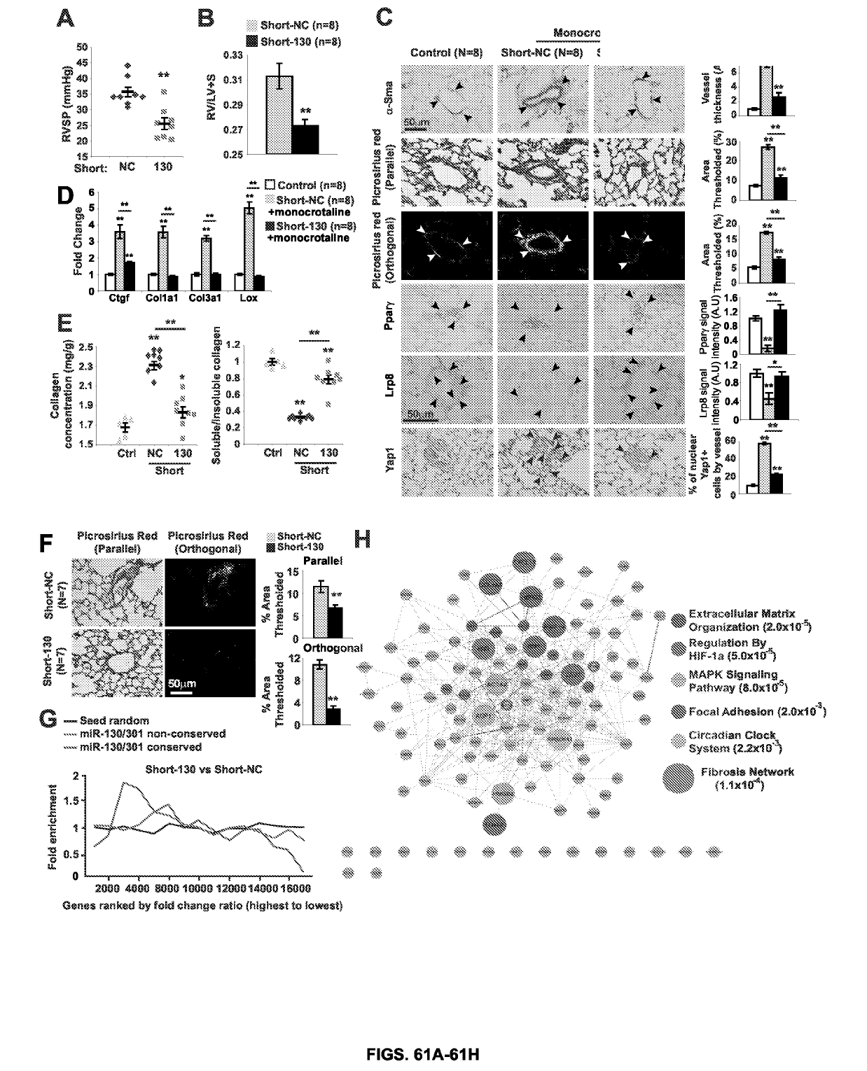

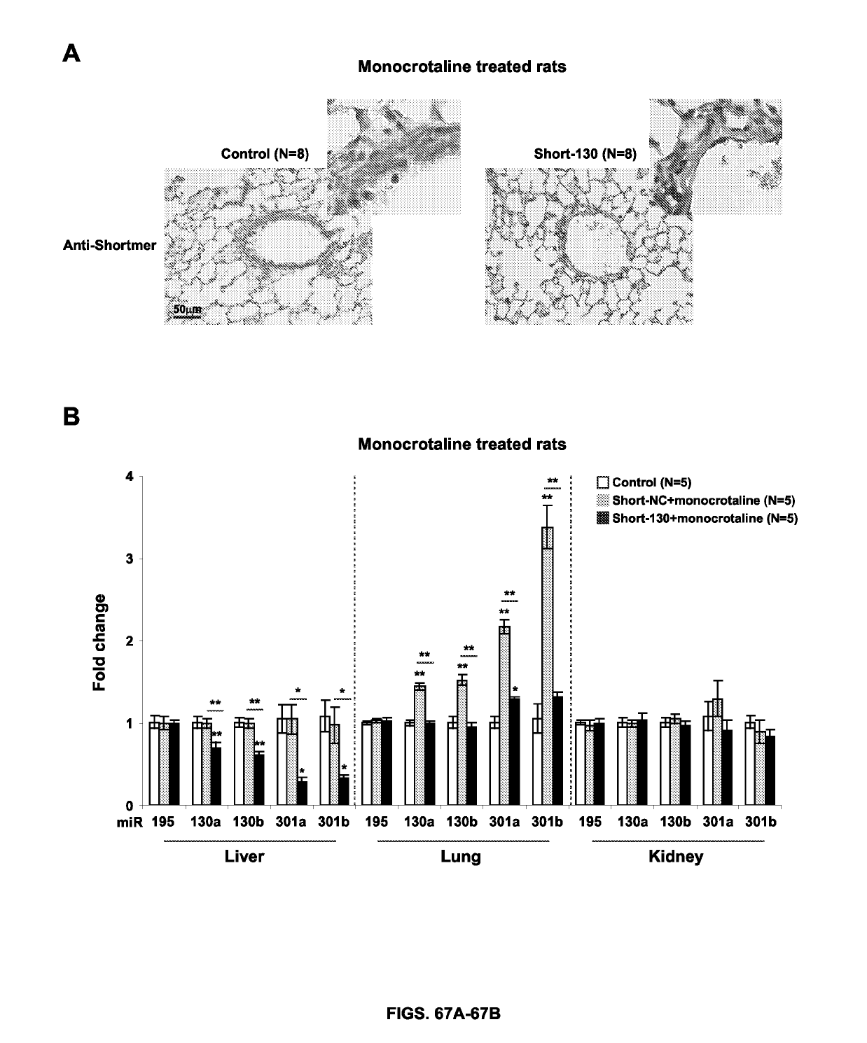

To determine whether inhibition of miR-130/301 ameliorates vessel fibrosis, hypoxia+SU5416-exposed mice and monocrotaline-exposed rats were examined (FIGS. 61A-61H). Rodents were serially administered a “shortmer” oligonucleotide recognizing the miR-130/301 seed sequence (Short-130), thus inhibiting all miR-130/301 members in vivo, as demonstrated in mouse lung (Bertero et al., 2014b) and rat lung (FIGS. 67A and 67B). In monocrotaline-exposed rats, Short-130 markedly reduced hemodynamic and histologic severity of PH (FIG. 61A-C) and reversed the decrease of Lrp8 and Pparγ in diseased control pulmonary arterioles (Short-NC) (FIG. 61C). miR-130/301 inhibition also reversed collagen deposition and crosslinking (FIG. 6C-E) as well as reduced YAP nuclear localization (FIG. 61C) and YAP activation, as reflected by decreased Ctgf (FIG. 61D). In hypoxia+SU5416-exposed mice, miR-130/301 inhibition also reversed collagen deposition and crosslinking (FIG. 61F). Furthermore, although whole lung transcriptomics likely captured only a subset of the miR-130/301 targets affecting the diseased pulmonary vasculature, transcriptomic analyses of whole lung from mice with hypoxia+SU5416-induced PH revealed a generalized de-repression of miR-130/301 targets by Short-130 (FIG. 61G, Table 12). Pathway enrichment of genes revealed pronounced representation of several pathways known to be involved in fibrosis (FIG. 61H, Table 13). Thus, the miR-130/301 family induces a programmatic molecular shift toward the fibrotic pathophenotype in vivo. In sum, miR-130/301 is both necessary and sufficient to promote pulmonary vascular stiffening in PH via, at least, a partially LOX-dependent manner.

Either Pharmacologic Activation of APOE with LXR Agonist GW3965 or Pharmacologic Inhibition of LOX-Dependent Collagen Crosslinking Decreases Peri-Arteriolar Fibrosis and Improves PH In Vivo.

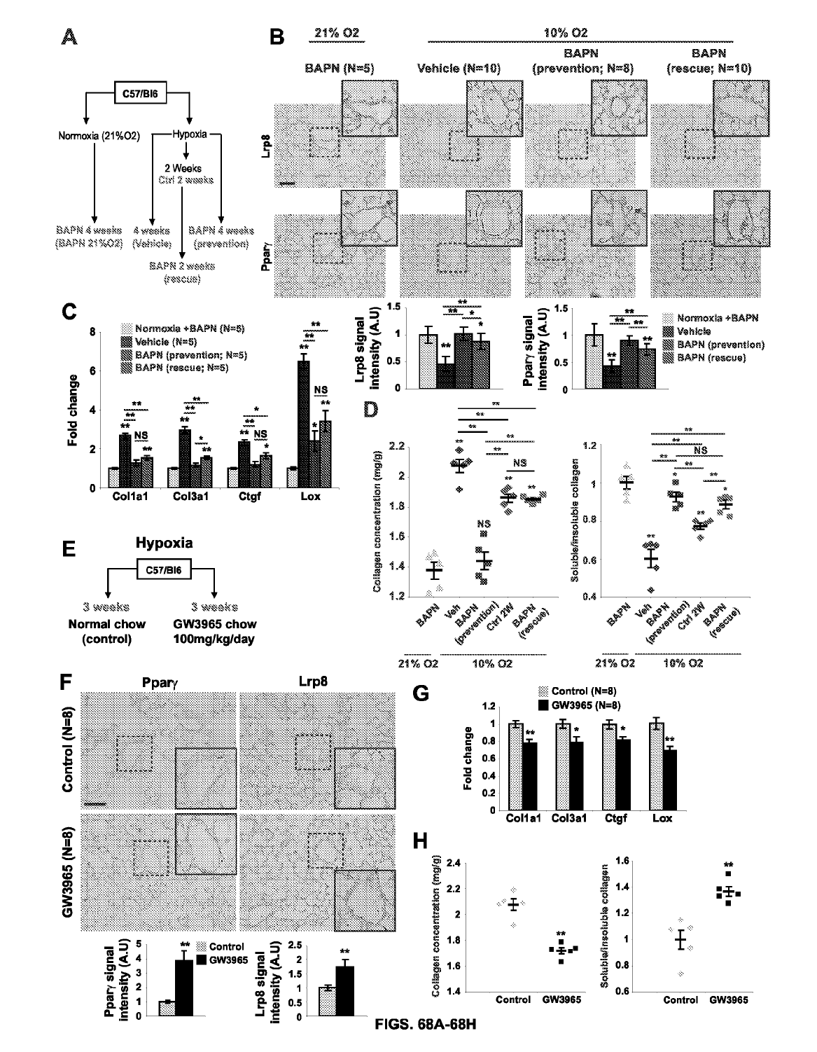

Finally, we wanted to determine if ApoE activity and/or LOX-dependent vascular stiffening were essential for ECM stiffening and PH development by miR-130/301. To assess the importance of ApoE activity, we attempted to prevent PH in mice via treatment with daily dietary ingestion of the liver-X nuclear hormone receptor (LXR) agonist GW3965, a known pharmacologic inducer of ApoE (Pencheva et al., 2014), simultaneously with hypoxia (FIGS. 62A-62H and 68A-H). Alternatively, experiments were designed to either prevent or reverse hypoxia-induced PH in mice via serial treatment with a pharmacologic inhibitor of LOX (Levental et al., 2009), β-aminopropionitrile BAPN, administered either simultaneously with hypoxia exposure (“prevention”) or after disease development (“reversal”) (FIGS. 62A-62H and 68A-H). In all cases of GW3965 or BAPN treatment, collagen crosslinking (FIG. 62D, 62H, FIG. 68) was inhibited, leading to decreased indices of PH, as reflected by RVSP (FIG. 62A, 62E), right ventricular remodeling (RV/LV+S ratio; FIG. 62B, 62F) and pulmonary arteriolar muscularization (FIG. 62C, 62G). GW3965 or BAPN treatment also reversed Pparγ and Lrp8 down-regulation (FIGS. 68A-68H) as well as reduced YAP nuclear localization (FIG. 62D, 62H) and YAP activation, as reflected by decreased Ctgf (FIGS. 68A-68H). Thus, considering the convergent data regarding pharmacologic manipulation of miR-130/301, APOE, and LOX, we can conclude that the YAP-TAZ-miR-130/301 circuit and its downstream network of targets act as master regulators of ECM remodeling in PH and represent a new set of targets for tailored molecular therapy in this disease.

### Discussion

In this study, we have found that mechanical forces act through a YAP/TAZ-miR-130/301 feedback loop to promote PH via vascular stiffening. This molecular circuit is active across diverse forms of PH—not only in acquired triggers of PH but also with factors genetically associated with this disease. Thus, this work highlights both the fundamental significance of ECM plasticity and the attractive potential of tailoring therapy to both this molecular circuit and the related downstream PPARγ-APOE-LRP8-LOX axis.

Our findings clarify the direct contribution of vascular ECM remodeling to PH pathogenesis and the molecular hierarchy promoting these pivotal events. Together, the early development of vascular fibrosis in numerous PH subtypes, the connection of ECM remodeling to factors that are genetically linked to PH, and the prevention and rescue of existing PH in mice with the LOX inhibitor BAPN define ECM remodeling as an initiating origin of PH rather than merely a consequence. In particular, while many factors genetically associated with PH have known relevance in bone morphogenetic protein (BMP) signaling, the pathogenic actions of two more recently identified effectors, CBLN2 (Germain et al., 2013) and the potassium channel KCNK3 (Chaumais et al., 2013), had not been linked to BMP-relevant factors or even to the same functional pathway. Thus, we envision that any one of several genetic mutations in these factors may activate the YAP/TAZ-miR-130/301 circuit, triggering a self-amplifying feedback process that reinforces pulmonary vascular stiffening and PH. Future work will be imperative to determine if other genomic polymorphisms associated with ECM biology are also linked to hereditary PAH. Moreover, given our prior findings that miR-130/301 members are also up-regulated by hypoxia and inflammatory cytokines (Bertero et al., 2014a; Bertero et al., 2014b), the “two-hit” hypothesis in hereditary PAH (Chan and Loscalzo, 2008b) could be explained in part by convergent effects of disease triggers causing additive or synergistic amplification of miR-130/301-specific pathogenic actions. The miR-130/301 family is also the first “mechanosensitive” miRNA family reported in a context beyond cancer and metastasis (Chou et al., 2013; Mouw et al., 2014). Given its adjustable, feedback-driven properties, the YAP/TAZ-miR-130/301 circuit may be partly responsible for individualized “tuning” of ECM remodeling depending upon each PH subtype. Moreover, given that YAP/TAZ are also induced by shear stress (Kim et al., 2014), miR-130/301 may also be responsive to increased pulmonary vascular flow such as in cases of congenital heart disease where PAH secondary to shunting predominates.

Distal to the YAP/TAZ-miR-130/301 circuit, vascular stiffening and its molecular controls may induce a number of independent downstream molecular processes promoting PH progression. Beyond its direct hemodynamic effects on pulmonary arterial pressure, arteriolar stiffening may also promote inflammatory signaling, cellular proliferation, apoptosis resistance, and cellular migration, as reported in cancer (Lu et al., 2012). The implication of PPARγ and APOE in ECM remodeling here in PH and prior reports of these molecules in PH pro-proliferative phenotypes (Bertero et al., 2014b; Hansmann et al., 2008) provide molecular support for such overlapping pathogenic processes. It is possible that naturally occurring loss-of-function APOE mutations, such as APOE4, may predispose to PH development. Alternatively, given its importance in the migration of metastatic cancer cells (Pencheva et al., 2014; Pencheva et al., 2012), the APOE-LRP8 partnership may also control vascular cell migration in PH. Finally, beyond the PPARγ-APOE-LRP8 axis, it is possible that an even more complex and wide-reaching interactome exists among miR-130/301, additional miRNAs, and their targets, all coincident with large portions of the same fibrotic gene network (FIG. 57A).

Finally, the applied paradigm of the YAP/TAZ-miR-130/301 circuit in ECM remodeling may greatly advance currently suboptimal clinical management strategies in PH. From a diagnostic perspective, the identification of the essential functions of YAP/TAZ and miR-130/301 that span multiple PH subtypes suggests the potential ability to identify new populations at risk for PH via targeted molecular screening of such miRNA activity. From a therapeutic perspective, the YAP/TAZ-miR-130/301 circuit and downstream effectors represent multiple promising pharmacologic targets in PH. Such tailored therapeutics represent a distinct advance from the current medical paradigms of treating PH solely with vasodilators. First, the prospect of targeting ECM plasticity may usher in a new era of addressing an overarching and early initiating origin of PH rather than just the end-stage consequence of disease. Second, our findings indicate the utility of modulating multiple, independent points in the same functional pathway and thus suggest the potential of additive or synergistic combination for more robust effect on downstream PH manifestations. As such, the application of shortmers (Obad et al., 2011) in vivo could impact substantially the rational treatment of pathogenic vascular stiffness by inhibiting multiple, but related, miRNAs rather than just a single miRNA. Our findings also indicate that factors downstream of YAP/TAZ and miR-130/301 may represent additional PH targets, thus allowing for a re-purposing of known pharmacologic agents that have only been applied previously to diseases beyond PH. For instance, consistent with prior work (Nave et al., 2014), our findings demonstrate the utility of LOX inhibition for ameliorating PH (FIGS. 62A-62G and 68A-68H), mirroring the beneficial effects of BAPN found in other pathologic circumstances of ECM stiffening such as tumor angiogenesis and neurodegenerative diseases (Rodriguez et al., 2008). Notably, chronic inhibition of LOX-dependent activity will necessitate greater tissue and temporal specificity, as it can adversely affect normal cardiovascular, neurologic, and pulmonary tissue development (Maki et al., 2005; Wilmarth and Froines, 1992) if utilized systemically and in the absence of an otherwise distinct ECM-relevant pathology. Our data also reveal that the LXR agonist GW3965 ameliorates ECM stiffening and PH manifestations in vivo (FIGS. 62A-62G and 68A-68H). While the clinical use of LXR agonists thus far have focused mainly on their roles in lipid and cholesterol metabolism in atherosclerosis, diabetes, and inflammation (Im and Osborne, 2011), application of their actions in ECM remodeling has yet to be explored in any context beyond cancer metastasis (Pencheva et al., 2014). Finally, recent descriptions of cyclic YAP-like peptides that interrupt YAP-TEAD interactions in oncogenesis could also carry therapeutic potential in PH (Zhou et al., 2014). Thus, with repression of miR-130/301, a combination of pharmacologic alterations of LOX, APOE, YAP/TAZ, and even PPARγ [e.g., rosiglitazone as described in (Bertero et al., 2014b)] could have synergistic effects on ECM-specific PH manifestations via therapeutic mechanisms that have yet to be exploited to this point.

In total, our findings highlight the importance of ECM stiffening in PH and define the control of a fibrotic gene network by the YAP/TAZ-miR-130/301 molecular circuit. Coupled with the feasibility of manipulating the expression of miR-130/301 family, APOE, and LOX in the pulmonary vasculature, these results may herald a new generation of cooperative therapeutic approaches that ameliorate vascular stiffness and thus prevent or reverse multiple forms of human PH.

### Experimental Procedures

Inhibition of miR-130/301 in a Rat Model of Monocrotaline-Induced PH:

Male Sprague-Dawley rats (10-14 week old) were injected with 60 mg/kg monocrotaline at time 0 followed by 5 intraperitoneal injections (every 3 days) of control or miR-130/301 shortmer oligonucleotides (20 mg/kg/dose; Regulus). Three days after the last injection, right heart catheterization was performed followed by harvest of lung tissue for RNA extraction or paraffin embedding.

RNA-Sequencing:

Illumina's TruSeq RNA Sample Preparation v2 kit was used to construct libraries for the samples accordingly to the manufacturer protocol. Briefly, 150 ng of total RNA input was used. The libraries were quantified using KAPA library quantification kit. Unstranded, paired-end sequencing was then done on Illumina HiSeq 2000 at 50 cycles/base-pair to generate 50 bp paired-end reads. Around 14˜19 million reads per sample were generated. Experimental data and associated designs were deposited in the NCBI Gene Expression Omnibus (http://www.ncbi.nlm.nih.gov/geo/) under series GSE61828.

Study Approval:

All animal experiments were approved by the Harvard Center for Comparative Medicine, the University of Colorado, Denver, and the Committees on Animal Research of the University of California, San Francisco. All experimental procedures involving the use of human tissue were approved by Institutional Review Boards at Partners Healthcare, Boston Children's Hospital, University of California, Los Angeles, and National Institutes of Health, as well as the New England Organ Bank. Ethical approval for this study conformed to the standards of the Declaration of Helsinki. Informed consent was obtained for right heart catheterization. For formalin-fixed paraffin-embedded lung samples, human PH specimens were collected from unused or discarded surgical samples as we previously described; non-diseased human lung specimens from the New England Organ Bank have been described (Kajstura et al., 2011).

Statistics:

Cell culture experiments were performed at least three times and at least in triplicate for each replicate. The number of animals in each group was calculated to measure at least a 20% difference between the means of experimental and control groups with a power of 80% and standard deviation of 10%. The number of unique patient samples for this study was determined primarily by clinical availability. In situ expression/histologic analyses of both rodent and human tissue, and pulmonary vascular hemodynamics in mice and rats were performed in a blinded fashion. Numerical quantifications for in vitro experiments using cultured cells or in situ quantifications of transcript/miRNA expression represent mean±standard deviation (SD). Numerical quantifications for physiologic experiments using rodents or human reagents represent mean±standard error of the mean (SEM). Immunoblot images are representative of experiments that have been repeated at least three times. Micrographs are representative of experiments in each relevant cohort. Paired samples were compared by a 2-tailed student's t test. A P-value less than 0.05 was considered significant. Correlation analyses were performed by Pearson correlation coefficient calculation.

Network Construction:

To construct the initial fibrosis network, we manually curated a set of 133 genes known fibrotic genes. Rather than compile a set of all genes known be modulated in fibrotic disease, we focused only on genes known to play a causative role in tissue fibrosis, through their involvement in matrix deposition and degradation, collagen crosslinking, and other fibrotic cellular processes. As previously described (Bertero et al., 2014), interactions between curated genes were annotated according to a master list of protein-protein, protein-DNA, kinase-substrate, and metabolic interactions, drawn from several consolidated databases, referred to here as the “consolidated interactome” (Parikh et al., 2012). In order to capture any additional fibrotic genes that may have been missed in our initial curation, we also incorporated a select number of non-curated genes demonstrated to interact with a significant number of genes in our curated set. As previously described (Bertero et al., 2014), this was performed by first defining a set of “fibrosis interactors.” An interactor was defined as any node in the consolidated interactome that (a) directly interacted with at least one member of our curated fibrosis gene set, but (b) was not itself a member of the curated set. Fibrosis interactors were then iteratively ranked by their shortest-path betweenness centrality score, considering only shortest paths between genes defined as “fibrotic” (initially members of the curated fibrosis set). With each iteration, the highest scoring node was incorporated into the fibrosis network, and defined as “fibrotic” on the next iteration. As discussed below, this same method was used to expand each of the 137 disease-specific networks. In order to prevent comparison of excessively large datasets, when the normalized betweenness centrality score of the highest scoring interactor fell below a fixed threshold of 0.10 (or, in the case of the disease-specific networks, when the number of incorporated nodes grew to twice the size of the original number of seed genes), no further nodes were added to the network. The resulting fibrosis network contained 350 nodes and 1459 edges, with a largest connected component of 339 nodes.

Network Clustering:

Clustering was performed using the Louvain method for community detection (Blondel et al., 2008), as implemented in the NetworkX package for Python 3.3. The final partition was selected so as to maximize modularity in the graph.

miRNA Target Prediction:

miRNA target prediction was performed using the TargetScan 6.2 (Conserved) algorithm (Friedman et al., 2009). The TargetScan algorithm detects mRNA with conserved complimentarily to the “seed” (nucleotides 2-7) of a given miRNA. Because of this, miRNA that share a seed are grouped together as a family and regarded as a single unit by the algorithm. For this reason, we do not distinguish between miRNA belonging to the same family in any of our statistical analyses.

miRNA Spanning Score:

In order to rank the influence of miRNA families on the fibrosis network (and other disease networks), we ranked miRNAs based on their “spanning score” (see also Table 11). As previously described (Bertero et al., 2014), this metric scores a miRNA family on three criteria: (1) the number of network genes which it targets, (2) the number of network clusters in which its targets reside, and (3) the hypergeometric p-value for the overlap of its target pool with the network. Each of these criteria is scored relatively, as a fraction of the maximum value achieved by any miRNA in our dataset for the network under consideration. This method provides a holistic assessment of the influence of a miRNA family on a given network of genes, considering not only the size, but also the spread and statistical significance of its target pool within the network.

Cell Culture and Reagents:

Primary human pulmonary arterial adventitial fibroblast cells (PAAFs) were purchased (ScienCell Research Laboratories) and grown in FGM cell culture media (Lonza). Experiments were performed at passages 3 to 6. Primary human pulmonary arterial endothelial cells (PAECs) were grown in EGM-2 cell culture media (Lonza), and experiments were performed at passages 3 to 6. Primary human pulmonary arterial smooth muscle cells (PASMCs) were cultured in SmGM-2 cell culture media (Lonza), and experiments were performed at passages 3 to 9. HEK293T cells (American Type Culture Collection) were cultivated in DMEM containing 10% fetal bovine serum (FBS). All cells were grown in collagen-coated plastic (50 ug/mL) at 37° C. in a humidified 5% CO₂ atmosphere. Collagen-coated hydrogel was purchased from Matrigen. Recombinant ApoE was purchased from PeproTech and used at a concentration of 5 μM.

Oligonucleotides and Transfection:

Pre-miRNA oligonucleotides (pre-miR-130a, negative control pre-miR-NC1, and premiR-NC2) and custom-designed tiny LNA oligonucleotides (tiny-130: 5′-ATTGCACT-3′ and tiny-NC: 5′-TCATACTA-3′ (SEQ ID NO: 51) were purchased from Life Technologies/Ambion and Exiqon, respectively. siRNAs for PPARγ, OCT4/POU5F1, and scrambled controls were purchased from Santa Cruz Biotechnology. On Target Plus siRNAs for YAP, TAZ (WWTR1), LRP8, and scrambled control were purchased from Thermo Fischer Scientific. PAAFs, PAECs, and PASMCs were plated in collagen-coated plastic (50 ug/mL) and transfected 24 h later at 70-80% confluence using pre-miRNA (5 nM), tiny-LNA (20 nM), or siRNA (25 nM) and Lipofectamine 2000 reagent (Life Technologies), according to the manufacturers' instructions. Eight hours after transfection, cells were trypsinized and re-plated in hydrogel.

Three-Dimensional Cell Culture:

This protocol was adapted from a prior publication (Aragona et al., 2013). Cells were embedded in a mixture of Growth Factor Reduced Matrigel (BD Biosciences) and Collagen-I (BD Biosciences). Collagen-I solution was neutralized on ice with 0.1M NaOH in PBS and adjusted with 0.1N HCl to bring the pH of the solution to 7.5. The Collagen-I solution was then mixed on ice with Matrigel to obtain a final concentration of 1.2 mg/ml (soft matrix) or 3 mg/ml (stiff matrix). Cells were trypsinized, counted, and resuspended in growth medium. 1:1 (v/v) mixtures were generated of cells with ECM mixture. 6-well plates were pre-coated with 1 ml of cell-free 50% medium/50% ECM, followed by solidification of the gel at 37° C. in a humidified 5% CO₂ atmosphere. Cells were then seeded on the ECM gel. Subsequently, wells were supplemented with normal growth medium 0.5% FBS, which was changed every 2 days during experimentation

Plasmids:

The 3′UTR sequences for LRP8 was purchased as gene fragment blocks (IDT) and cloned in the pSI-CHECK-2 vector (Promega) downstream of the Renilla luciferase using XhoI and NotI restrictions sites. Mutagenesis of the putative miR-130/301 family binding sites was performed using gene fragment blocks containing mutations in positions 2, 4, and 6 of the “seed” biding site (IDT). The PPARγ coding sequence (BC006811) was amplified by polymerase chain reaction (PCR) using high-fidelity polymerase Phusion (Thermo Fisher Scientific) from an MGC cDNA clone (clone ID: 3447380) and inserted in the pCDH-CMV-MCS-EF1-copGFP (System Biosciences) using NheI and NotI restrictions sites. The lentiviral parent vector expressing GFP was used as a control. Stable expression of these constructs in PAAFs, PAECs, and PASMCs was achieved by lentiviral transduction. All cloned plasmids were confirmed by DNA sequencing.

Lentivirus Production:

HEK293T cells were transfected using Lipofectamine 2000 (Life Technologies) with lentiviral plasmids along with packaging plasmids (pPACK, System Biosciences), according to the manufacturer's instructions. Virus was harvested, sterile filtered (0.45 um), and utilized for subsequent infection of PAAFs, PAECs, and PASMCs (24-48 hour incubation) for gene transduction.

miRNA Target Validation by Luciferase Assay:

Adapted from our previously published protocol, HEK293T cells were plated in 96-well plates and transfected with 200 ng of pSICHECK-2 construct and 5 nM of pre-miRNAs using Lipofectamine 2000 (Life Technologies). The medium was replaced 8 hours after transfection with fresh medium containing 10% FCS, L-glutamine. 48 hours after transfection, firefly and Renilla Luciferase activities were measured using the Dual-Glo™ Luciferase assay (Promega).

Messenger RNA and miRNA Extraction:

Cells were homogenized in 1 ml of QiaZol reagent (Qiagen). Total RNA content, including small RNAs, was extracted using the miRNeasy kit (Qiagen) according to the manufacturer's instructions. Total RNA concentration was determined using a ND-1000 micro-spectrophotometer (NanoDrop Technologies).

Quantitative RT-PCR of Mature miRNAs:

Mature miRNA expression was evaluated using TaqMan MicroRNA Assays (Life Technologies/Applied Biosystems) and the Applied Biosystems 7900HT Fast Real Time PCR device (Life Technologies/Applied Biosystems). Expression levels were normalized to RNU48 or snoR55 for human or mouse experiments, respectively, and calculated using the comparative Ct method (2^{-ΔΔCt}).

Quantitative RT-PCR of Messenger RNAs:

Messenger RNAs were reverse transcribed using the Multiscript RT kit (Life Technologies) to generate cDNA. cDNA was amplified via fluorescently labeled Taqman primer sets using an Applied Biosystems 7900HT Fast Real Time PCR device. Fold-change of RNA species was calculated using the formula (2^{-Ct}), normalized to actin expression.

Immunoblotting and Antibodies:

Cells were lysed in Laemmeli buffer. Protein lysate were resolved by SDS-PAGE and transferred onto a PVDF membrane (Biorad). Membranes were blocked in 5% non-fat milk in TN buffer (50 mM Tris-HCl pH 7.4, 150 mM NaCl) or 5% BSA in TN buffer and incubated in the presence of the primary and then secondary antibodies. After washing in TN buffer containing 0.1% Tween, immunoreactive bands were visualized with the ECL system (Amersham Biosciences). A primary antibody for YAP/TAZ (#8418; 1/1000) was obtained from Cell Signaling. Primary antibodies for APOE (ab1906; 1/100), LRP8 (ab108208; 1/1000), and Actin (ab 3280; 1/500) were obtained from Abcam. Appropriate secondary antibodies (anti-rabbit and anti-mouse) coupled to HRP were used (Santa Cruz Biotechnology).

Immunofluorescence:

After the different treatment cells were fixed with PBS/PFA 4% for 10 min and permeabilized with PBS/Triton 100×0.1% for 10 min. Then cells were incubated with anti-YAP1 (#4912; 1/100; Cell signaling) at 4° C. overnight. Secondary antibodies coupled with Alexa-594 (Life Technologies) were used at 1:500. Nuclei were counterstained with DAPI (Sigma).

ECM Remodeling Experiment:

Following treatment, PAAFs were plated atop soft matrices (see below) in normal growth medium supplemented with 0.5% FBS. After three days, 10 ug/mL of puromycin was added to the growth medium for 48 hours to detach the cells from the matrix. Matrices were washed 3 times (PBS). Naïve PAAFs were then plated on top of these matrices supplemented with normal growth medium 0.5% FBS or harvested for assessment of collagen crosslinking (Picrosirius Red stain). Sixteen hours later, cells were harvested for immunofluorescence experiments or RNA extraction.

Animals:

All animal treatments and analyses were conducted in a controlled and non-blinded manner.

Pulmonary Hypertension Models:

Eight-week-old littermate mice (C57Bl6) were exposed serially to SU5416 or vehicle control (20 mg/kg/dose/week; Sigma-Aldrich), followed by exposure to normobaric hypoxia (10% O2; OxyCycler chamber, Biospherix Ltd, Redfield, N.Y.) or normoxia (21% O2) for 3 weeks.

Male Sprague-Dawley rats (10-14 week old) were injected with 60 mg/kg monocrotaline at time 0. At 0-4 weeks post-exposure, right heart catheterization was performed followed by harvest of lung tissue for RNA extraction or paraffin embedding.

VHL flox/flox; Cre-ER mice (C57Bl6 background, >10 backcrosses) were a generous gift from W. G. Kaelin (Dana Farber Cancer Institute, Boston). Conditional inactivation of VHL was performed by treating 3-week-old VHL flox/flox, Cre-ER mice with 2 mg of tamoxifen (Sigma Aldrich), administered via intraperitoneal injection every other day for 2 doses, as previously described (Chan et al., 2009), followed by tissue harvest at 10-13 weeks of age. Tissue from tamoxifen-treated, gender-(male), and age-matched VHL flox/flox mice (without the Cre-ER transgene) was used as wildtype comparison (referred to as VHL WT).

IL-6 transgenic mice (C57Bl6 background) have been described previously (Steiner et al., 2009).

Schistosoma mansoni-infected mice: Mice were exposed to S. mansoni ova to cause experimental PH, using a published technique (Graham et al., 2013a; Graham et al., 2013b). Eight week old C57Bl6/J mice were used for the study. Experimentally exposed mice were intraperitoneally sensitized on day one and then intravenously challenged on day 14 with S. mansoni eggs, at a dose of 175 eggs/gram body weight at each time point. S. mansoni eggs were obtained from homogenized and purified livers of Swiss-Webster mice infected with S. mansoni cercariae, provided by the Biomedical Research Institute (Rockville, Md.). On day 21, right ventricular catheterization was performed followed by tissue and blood collection. The mice were anesthetized with ketamine/xylazine and ventilated through a transtracheal catheter. The abdominal and thoracic cavities were opened, and a 1Fr pressure-volume catheter (Millar PVR-1035, Millar Instruments) was placed through the right ventricle apex to transduce the pressure. Blood samples (400 μl) were drawn after catheterization using into a syringe containing 100 μL 0.5M EDTA at pH 8.0. Blood samples were centrifuged at 2000 g for 20 minutes at 4° C. to separate plasma. The remaining blood was flushed out of the lungs with PBS, the right bronchus was sutured, and 1% agarose was instilled into the left lung through the transtracheal catheter. The left lung was removed, formalin-fixed, and paraffin-embedded for histology. The right lungs were removed and snap frozen. The right ventricle free wall was dissected off of the heart and weighed relative to the septum and left ventricle to measure hypertrophy (the Fulton index).

Male Sprague-Dawley rats (10-14 week old) were injected with SU5416 (20 mg/kg; Sigma-Aldrich), followed by exposure to normobaric hypoxia (10% O2; OxyCycler chamber, Biospherix Ltd, Redfield, N.Y.) or normoxia (21% O2) for 3 weeks. Right heart catheterization was performed, followed by harvest of lung tissue for RNA extraction or paraffin embedding.

Surgical placement of PA-aortic shunts in juvenile lambs: As previously described (Reddy et al., 1995), pregnant mixed-breed Western ewes (137-141 d gestation, term=145 d) were anesthetized. Fetal exposure was obtained through the horn of the uterus; a left lateral thoracotomy was performed on the fetal lamb. With the use of side-biting vascular clamps, an 8.0-mm vascular graft was anastomosed between the ascending aorta and main pulmonary artery of the fetal lambs. Four weeks after spontaneous delivery shunt and control (provided by twin pregnancy or age-matched) lambs were anesthetized and catheters were placed to measure hemodynamics including left pulmonary blood flow. After baseline hemodynamics were obtained, peripheral lung was obtained for analysis. At the end of the protocol, all lambs were euthanized with a lethal injection of sodium pentobarbital followed by bilateral thoracotomy as described in the National Institutes of Health Guidelines for the Care and Use of Laboratory Animals.

Forced Pulmonary Expression of miR-130a in Lungs of Mice In Vivo:

Eight-week-old mice (C57Bl6) were injected with SU5416 (20 mg/kg; Sigma-Aldrich), followed by 4 intrapharyngeal injections (once by week) of 1 nmol of miR-control (pre-miR-NC) or miR-130a (pre-miR-130a) mixed in 100 uL PBS solution containing 5% Lipofectamine 2000 (Life Technologies). Such intrapharyngeal injections led to effective delivery to the pulmonary arterioles, as we previously described in detail 7. BAPN (Sigma-Aldrich) was dissolved in water and administered daily in indicated mouse cohorts (30 mg/kg/day). Three days after the last oligonucleotide injection, right heart catheterization was performed as previously described (Parikh et al., 2012), followed by harvest of lung tissue for RNA extraction or paraffin embedding, as described above.

Treatment of Mice with Oral Ingestion of the Liver-X Nuclear Hormone Receptor (LXR) Agonist GW3965:

To determine the effects of the LXR agonist GW3965 (Sigma-Aldrich) and consequent APOE induction on PH development, as previously described (Pencheva et al., 2014), mice were exposed to normobaric hypoxia and simultaneously assigned to control chow or chow supplemented with GW3965 (Research Diets, Inc.) at doses of 100 mg of drug per kilogram of mouse per day (based on average daily intake of 3.5 μm of chow). After three weeks, right heart catheterization was performed followed by harvest of lung tissue for RNA extraction or paraffin embedding.

Picrosirius Red Stain and Quantification:

Picrosirius Red stain was achieved through the use of 5 μm paraffin sections stained with 0.1% Picrosirius Red (Direct Red80, Sigma-Aldrich) and counterstained with Weigert's hematoxylin to reveal fibrillar collagen. The sections were then serially imaged using with an analyzer and polarizer oriented parallel and orthogonal to each other. Microscope conditions (lamp brightness, condenser opening, objective, zoom, exposure time, and gain parameters) were constant throughout the imaging of all samples. A minimal threshold was set on appropriate control sections for each experiment in which only the light passing through the orthogonally-oriented polarizers representing fibrous structures (i.e., excluding residual light from the black background) was included. The threshold was maintained for all images across all conditions within each experiment. The area of the transferred regions that was covered by the thresholded light was calculated and at least 10 sections/vessel per condition were averaged together (Image J software).

Measurement of Collagen Content in Lung Tissue:

This protocol was adapted from a previously published protocol (Hu et al.). Mouse lung was weighed, minced, and incubated in 0.5 M acetic acid at 4° C. After overnight digestion, the acetic acid-soluble and insoluble fractions were isolated by centrifugation. The soluble fraction was stored at −80° C., while the insoluble fraction was digested by overnight incubation in 6M hydrochloric acid at 85° C. Concentrations of soluble and insoluble (gelatinous) collagen fractions were determined using a Sircol Soluble Collagen Assay Kit (Biocolor) with a colorimetric reaction (measured at 550 nm) and a provided collagen reference standard curve.

Inhibition of miR-130/301 in a Mouse Model of PH:

Eight-week-old mice (C57Bl6) were injected with SU5416 (20 mg/kg/dose/week; Sigma-Aldrich), followed by exposure to normobaric hypoxia (10% O2; OxyCycler chamber, Biospherix Ltd, Redfield, N.Y.) for 2 weeks. After two weeks and confirmation of PH development in five mice (right heart catheterization), mice were further treated with hypoxia+SU5416, along with 3 intrapharyngeal injections (every 4 days) of control or miR-130/301 shortmer oligonucleotides, designed as fully modified antisense oligonucleotides complementary to the seed sequence of the miR-130/301 miRNA family (10 mg/kg; Regulus). Specifically, the control and miR-130/301 shortmer oligonucleotides were non-toxic, lipid-permeable, high-affinity oligonucleotides. The miR-130/301 shortmer carried a sequence complementary to the active site of the miR-130/301 miRNA family, containing a phosphorothioate backbone and modifications (fluoro, methoxyethyl, and bicyclic sugar) at the sugar 2′ position. Such intrapharyngeal injections led to effective delivery to the pulmonary arterioles, as we previously described in detail (7). Three days after the last injection, right heart catheterization was performed followed by harvest of lung tissue for RNA extraction or paraffin embedding, as described above.

Lung Tissue Harvest:

After physiological measurements by direct right ventricular puncture, the pulmonary vessels were gently flushed with 1 cc of saline to remove the majority of blood cells, prior to harvesting cardiopulmonary tissue. The heart was removed, followed by dissection and weighing of the right ventricle (RV) and of the left ventricle+septum (LV+S). Organs were then harvested for histological preparation or flash frozen in liquid N2 for subsequent homogenization and extraction of RNA and/or protein. To further process lung tissue specifically, prior to excision, lungs were flushed with PBS at constant low pressure (˜10 mmHg) via right ventricular cannulation, followed by tracheal inflation of the left lung with 10% neutral-buffered formalin (Sigma-Aldrich) at a pressure of ˜20 cm H₂O. After excision and 16 hours of fixation in 10% neutral-buffered formalin at 25° C., lung tissues were paraffin-embedded via an ethanol-xylene dehydration series, before being sliced into 5 μm sections (Hypercenter XP System and Embedding Center, Shandon).

In situ hybridization: The protocol for in situ hybridization for miRNA detection was based on a prior report (Bertero et al., 2014). Specifically, 5 μm tissues sections were probed using a 3′ fluorescein isothiocyanate (FITC) labeled miRCURY LNA hsa-miR-130a detection probe (Exiqon). The miRCURY LNA scramble-miR probe was used as negative control. Following re-hydration (Sigma) tissues were formaldehyde-fixed (4% formaldehyde, Sigma) before inactivation of endogenous enzymes by acetylation buffer [873 uL of triethanolamine (Sigma) and 375 uL acetic anhydride (Fisher) in 75 ml distilled water]. Probe annealing (25 nM LNA probe) was performed in hybridization buffer (Sigma, H7782) for 16 hours at RNA-Tm-22° C. (62° C.). Following serial washes with 2×SSC, 1×SSC, and 0.5×SSC (Sigma) at 62° C., immunolabeling was performed with an anti-FITC biotinconjugated antibody for overnight at 4° C. (1:400; Sigma-Aldrich). For detection, development was achieved by adding streptavidin-biotinylated alkaline phosphatase complex (Vector Labs) followed by Nitro blue tetrazolium chloride/5-Bromo-4-chloro-3-indolyl phosphate substrate solution (NBT/BCIP, Roche), and positive staining was evident by a blue color. MiR-130a expression was quantified in the vascular wall of 15-20 pulmonary arteries (<100 μm external diameter in rodents and <200 μm external diameter in humans) using ImageJ software (NIH).

Immunohistochemistry of Lung:

Lung sections (5 μm) were deparaffinized and high temperature antigen retrieval was performed, followed by blocking in TBS/BSA 5%, 10% goat serum and exposure to primary antibody and biotinylated secondary antibody (Vectastain ABC kit, Vector Labs). A primary antibody against YAP1 (#4912; 1/200) was obtained from Cell Signaling. Primary antibodies against, LRP8 (ab115196; 1/100), α-SMA (1/400) were purchased from Abcam. Primary antibodies against OCT4 (sc-5279; 1/50) and PPARγ (sc-7273; 1/50) were purchased from Santa Cruz. Primary antibody against anti-shortmer (E5746-B3A; 1/1000) was provided by Regulus Therapeutics. In most cases, color development was achieved by adding streptavidin-biotinylated alkaline phosphatase complex (Vector Labs) followed by Vector Red alkaline phosphatase substrate solution (Vector Labs). Levamisole was added to block endogenous alkaline phosphatase activity (Vector Labs). Pictures were obtained using an Olympus Bx51 microscope. Small pulmonary vessels (<100 μm diameter) present in a given tissue section (>10 vessels/section) that were not associated with bronchial airways were selected for analysis (N>5 animals/group). Intensity of staining was quantified using ImageJ software (NIH). Degree of pulmonary arteriolar muscularization was assessed in paraffin-embedded lung sections stained for α-SMA by calculation of the proportion of fully and partially muscularized peripheral (<100 μm diameter) pulmonary arterioles to total peripheral pulmonary arterioles, as previously described (Hansmann et al., 2007). Medial thickness was also measured in α-SMA stained vessels (<100 μm diameter) using ImageJ software (NIH) and expressed as arbitrary units. All measurements were performed blinded to condition.

RNA-Seq Data Pre-Processing and Analysis:

FastQC (FastQC), a quality control tool for high throughput sequence data, was used for checking q-scores and reads were not trimmed prior to alignments. The data were aligned against UCSC mouse genome version mm10 using STAR aligner (Dobin et al., 2013). Then, from the BAM files, gene-level counts of uniquely mapped reads were computed using SAMTools (Li et al., 2009a) and HTSeq v0.5.4p5 (Smyth et al., 2005). The gene level counts were then normalized with the R/Bioconductor package limma(Smyth et al., 2005) using the voom (Law et al., 2014)/variance stabilization method. The data were quality controlled for outliers using principal component analysis (PCA). Differential expression analysis between transcriptome profiles of experimental groups was performed using the R/Bioconductor package limma(Smyth et al., 2005) that includes methods for RNA-Seq Data analysis.

Statistical Analysis:

Transcriptomic analysis was performed in the lungs of mice exposed to hypoxia+SU5416 and treated with either Short-NC (n=3) or Short-130 (n=3) as described above, as compared with mice exposed to normoxia+SU5416. Genes that showed robust modulation in the context of hypoxia (p<0.05) and modulation in the opposite direction when miR-130/301 was suppressed (p<0.15) were cross-referenced with the consolidated interactome (CI) to form a network whose modulation under hypoxia was miR-130/301-dependent. Notably, we chose to use a less stringent p-value cutoff for modulation under conditions of miR-130/301 suppression, as these genes had already been subject to two prior screening criteria: (1) they were modulated in the context of hypoxia (p<0.05) and (2) hypoxia-induced effects were reversed when miR-130/301 was suppressed. As we believed these criteria to be fairly stringent on their own, we felt it was unnecessary to exclude genes with moderately low p-values if they met these other specifications.

Pathway Enrichment:

Pathway enrichment of this network was performed using the Reactome FI plug-in for Cytoscape 2.0. The network was cross-referenced with several databases (Kegg, Reactome, GO, NCBI PID, and Biocarta) collectively representing a broad range of functional pathways. Pathways were then ranked according to the hypergeometric p-value for their overlap with this miR-130/301-dependent network (as we have previously described (Parikh et al., 2012)).

Target Analysis.

De-repression of miR-130/301 target genes by Short-130 administration was assessed using the sylamer approach (van Dongen et al., 2008) as previously reported (Bertero et al., 2014).

TABLE 8
Pathway enrichment of genes modulated in whole lung of mice suffering from hypoxia +
SU5416-induced PH: Genes were screened in order to isolate those that showed robust
modulation under hypoxic conditions (p < 0.01). Pathway enrichment was performed
as previously described (Parikh et al., 2012), and functional pathways were ranked
according to the hypergeometric p-value of their overlap with the screened gene set.

| | | | GENES IN | # GENES IN | | |
|---|---|---|---|---|---|---|
| RANK | PATHWAY | DATABASE | PATHWAY | PATHWAY | PVAL | FDR |
| 1 | Focal adhesion | KEGG | PGF, PIK3CG, | 17 | <0.0001 | 1.00E−03 |
| | | | VEGFC, LAMB2, | | | |
| | | | LAMA2, FLNB, | | | |
| | | | RELN, ITGB7, | | | |
| | | | VAV3, VAV2, | | | |
| | | | ARHGAP5, PAK1, | | | |
| | | | COL4A4, ACTB, | | | |
| | | | COL4A2, COL5A3, | | | |
| | | | VWF | | | |
| 2 | Chemokine | KEGG | ADCY3, ADCY5, | 16 | <0.0001 | 5.00E−04 |
| | signaling | | ADCY6, GNG2, | | | |
| | pathway | | PIK3CG, CCR2, | | | |
| | | | GNGT2, WAS, | | | |
| | | | GNG10, CXCL12, | | | |
| | | | CX3CL1, VAV3, | | | |
| | | | VAV2, IKBKG, | | | |
| | | | PAK1, PLCB2 | | | |
| 3 | Leukocyte | KEGG | PIK3CG, NCF4, | 11 | <0.0001 | 3.33E−04 |
| | transendothelial | | CXCL12, VAV3, | | | |
| | migration | | VAV2, PLCG1, | | | |
| | | | PLCG2, ARHGAP5, | | | |
| | | | JAM2, ACTB, | | | |
| | | | CYBB | | | |
| 4 | Extracellular | Reactome | LTBP1, ADAMTS2, | 16 | <0.0001 | 2.50E−04 |
| | matrix | | LAMB2, LAMA2, | | | |
| | organization | | ADAM15, HSPG2, | | | |
| | | | FBLN2, DST, | | | |
| | | | ITGB7, LOXL2, | | | |
| | | | JAM2, COL8A1, | | | |
| | | | COL4A4, COL4A2, | | | |
| | | | FBN1, COL5A3 | | | |
| 5 | Regulation of | Reactome | ADCY3, ADCY5, | 7 | <0.0001 | 2.00E−04 |
| | Water Balance | | ADCY6, AQP1, | | | |
| | by Renal | | GNG2, GNGT2, | | | |
| | Aquaporins | | GNG10 | | | |
| 6 | PI3K-Akt | KEGG | PGF, GNG2, | 18 | <0.0001 | 1.67E−04 |
| | signaling | | PIK3CG, VEGFC, | | | |
| | pathway | | LAMB2, LAMA2, | | | |
| | | | GNGT2, NOS3, | | | |
| | | | GNG10, RELN, | | | |
| | | | ITGB7, NR4A1, | | | |
| | | | IKBKG, COL4A4, | | | |
| | | | COL4A2, IL2RB, | | | |
| | | | COL5A3, VWF | | | |
| 7 | ECM-receptor | KEGG | LAMB2, LAMA2, | 9 | <0.0001 | 1.43E−04 |
| | interaction | | HSPG2, RELN, | | | |
| | | | ITGB7, COL4A4, | | | |
| | | | COL4A2, COL5A3, | | | |
| | | | VWF | | | |
| 8 | Fc gamma R- | KEGG | PIK3CG, WAS, | 9 | <0.0001 | 3.75E−04 |
| | mediated | | FCGR2B, ARPC4, | | | |
| | phagocytosis | | VAV3, VAV2, | | | |
| | | | PLCG1, PLCG2, | | | |
| | | | PAK1 | | | |
| 9 | RAC1 signaling | NCBI | ACTR3, ARPC4, | 7 | <0.0001 | 5.56E−04 |
| | pathway | | ARHGAP5, PAK1, | | | |
| | | | PLCB2, RACGAP1, | | | |
| | | | CYBB | | | |
| 10 | Rap1 signaling | KEGG | ADCY3, PGF, | 13 | <0.0001 | 5.00E−04 |
| | pathway | | ADCY5, ADCY6, | | | |
| | | | PIK3CG, VEGFC, | | | |
| | | | SIPA1L1, FYB, | | | |
| | | | MRAS, VAV2, | | | |
| | | | PLCG1, PLCB2, | | | |
| | | | ACTB | | | |
| 11 | Endothelin | NCBI | ADCY3, ADCY5, | 7 | <0.0001 | 5.46E−04 |
| | signaling | | ADCY6, PIK3CG, | | | |
| | pathway | | EDNRA, NOS3, | | | |
| | | | PLCB2 | | | |
| 12 | Retrograde | KEGG | ADCY3, ADCY5, | 9 | <0.0001 | 9.17E−04 |
| | endocannabinoid | | ADCY6, GNG2, | | | |
| | signaling | | GNGT2, GNG10, | | | |
| | | | CACNA1C, MGLL, | | | |
| | | | PLCB2 | | | |
| 13 | Integration of | Reactome | ADCY3, ADCY5, | 9 | <0.0001 | 1.00E−03 |
| | energy | | ADCY6, GNG2, | | | |
| | metabolism | | GNGT2, GNG10, | | | |
| | | | KCNB1, CACNA1C, | | | |
| | | | PLCB2 | | | |
| 14 | Ras signaling | KEGG | PGF, GNG2, | 13 | <0.0001 | 1.07E−03 |
| | pathway | | PIK3CG, VEGFC, | | | |
| | | | GNGT2, GNG10, | | | |
| | | | MRAS, RASAL2, | | | |
| | | | PLCG1, PLCG2, | | | |
| | | | IKBKG, PAK1, | | | |
| | | | RASGRP4 | | | |
| 15 | Cholinergic | KEGG | ADCY3, ADCY5, | 9 | 0.0001 | 1.73E−03 |
| | synapse | | ADCY6, GNG2, | | | |
| | | | PIK3CG, GNGT2, | | | |
| | | | GNG10, CACNA1C, | | | |
| | | | PLCB2 | | | |
| 16 | Beta1 integrin | NCBI | LAMB2, LAMA2, | 7 | 0.0001 | 2.19E−03 |
| | cell surface | | F13A1, CSPG4, | | | |
| | interactions | | JAM2, COL4A4, | | | |
| | | | FBN1 | | | |
| 17 | Vascular smooth | KEGG | ADCY3, ADCY5, | 9 | 0.0001 | 2.71E−03 |
| | muscle | | ADCY6, EDNRA, | | | |
| | contraction | | MYH11, CACNA1C, | | | |
| | | | PLCB2, RAMP3, | | | |
| | | | CALD1 | | | |
| 18 | Regulation of | KEGG | PIK3CG, WAS, | 12 | 0.0001 | 2.61E−03 |
| | actin | | TMSB4X, IQGAP2, | | | |
| | cytoskeleton | | MRAS, ARPC4, | | | |
| | | | ITGB7, VAV3, | | | |
| | | | VAV2, PAK1, | | | |
| | | | RDX, ACTB | | | |
| 19 | Circadian | KEGG | ADCY3, ADCY5, | 8 | 0.0001 | 2.74E−03 |
| | entrainment | | ADCY6, GNG2, | | | |
| | | | GNGT2, GNG10, | | | |
| | | | CACNA1C, PLCB2 | | | |
| 20 | GPVI-mediated | Reactome | PIK3CG, FCER1G, | 5 | 0.0001 | 2.60E−03 |
| | activation | | VAV3, VAV2, | | | |
| | cascade | | PLCG2 | | | |
| 21 | Inflammation | NCBI | PIK3CG, ALOX5AP, | 7 | 0.0001 | 2.36E−03 |
| | mediated by | | ITGB7, PLCG1, | | | |
| | chemokine and | | PLCG2, PLCB2, | | | |
| | cytokine | | VWF | | | |
| | signaling | | | | | |
| | pathway | | | | | |
| 22 | Thyroid | KEGG | ADCY3, ATP1B2, | 7 | 0.0001 | 2.36E−03 |
| | hormone | | ADCY5, ADCY6, | | | |
| | synthesis | | GPX3, ATP1A3, | | | |
| | | | PLCB2 | | | |
| 23 | Beta5 beta6 | NCBI | EDIL3, CYR61, | 4 | 0.0001 | 2.65E−03 |
| | beta7 and beta8 | | ITGB7, FBN1 | | | |
| | integrin cell | | | | | |
| | surface | | | | | |
| | interactions | | | | | |
| 24 | MAPK signaling | KEGG | TGFBR2, NTRK2, | 13 | 0.0001 | 2.67E−03 |
| | pathway | | FLNB, MKNK2, | | | |
| | | | DUSP6, STMN1, | | | |
| | | | MRAS, MAP4K1, | | | |
| | | | NR4A1, IKBKG, | | | |
| | | | CACNA1C, PAK1, | | | |
| | | | RASGRP4 | | | |
| 25 | Gastric acid | KEGG | ADCY3, ATP1B2, | 7 | 0.0001 | 2.68E−03 |
| | secretion | | ADCY5, ADCY6, | | | |
| | | | ATP1A3, PLCB2, | | | |
| | | | ACTB | | | |

TABLE 9
Curated seed genes for the fibrosis network

| Gene | References |
|---|---|
| ACE | (Brilla et al., 1993) |
| ACTA2 | (Wynn and Ramalingam, 2012) |
| ACVR1 | (Grygielko et al., 2005) |
| ADORA2A | (Delia Latta et al., 2013) |
| AGT | (Fern et al., 1999) |
| AKT1 | (Liu et al., 2013) |
| APLN | (Reichenbach et al., 2012) |
| AQP1 | (Gao et al., 2013) |
| AQP3 | (Bedford et al., 2003) |
| BCL2 | (Safaeian et al., 2014) |
| BMP7 | (Boon et al., 2011) |
| CAV1 | (Gvaramia et al., 2013) |
| CAV2 | (de Almeida et al., 2013) |
| CCL11 | (Mangieri et al., 2012) |
| CCL2 | (Wynn and Ramalingam, 2012) |
| CCL3 | (Wynn and Ramalingam, 2012) |
| CCR2 | (Wynn and Ramalingam, 2012) |
| CEBPB | (Hu et al., 2012) |
| CFTR | (Liedtke, 1992) |
| COL1A1 | (Wynn and Ramalingam, 2012) |
| COL1A2 | (Wynn and Ramalingam, 2012) |
| COL3A1 | (Wynn and Ramalingam, 2012) |
| COL4A1 | (Wynn and Ramalingam, 2012) |
| COMP | (Kim et al., 2006) |
| CTGF | (Maher, 2012) |
| CTNNB1 | (Guo et al., 2012) |
| CXCL12 | (Tsutsumi et al., 2007) |
| CXCL6 | (Besnard et al., 2013) |
| CXCR4 | (Tsutsumi et al., 2007) |
| CYR61 | (Lai et al., 2014) |
| DCN | (Baghy et al., 2011) |
| EDN1 | (Swigris and Brown, 2010) |
| EGF | (Kisseleva and Brenner, 2011) |
| EGFR | (Kisseleva and Brenner, 2011) |
| ELN | (Shi et al., 2007) |
| ENG | (Maring et al., 2012) |
| F10 | (Wynn and Ramalingam, 2012) |
| FASLG | (Wynes et al., 2011) |
| GREM1 | (Murphy et al., 2002) |
| HAVCR1 | (Humphreys et al., 2013) |
| HGF | (Chakraborty et al., 2013) |
| IFNG | (Bonilla et al., 2006) |
| IGF1 | (Sokolovic et al., 2013) |
| IL10 | (Mandal et al., 2010) |
| IL13 | (Wynn and Ramalingam, 2012) |
| IL13RA2 | (Granel et al., 2007) |
| IL1A | (Gieling et al., 2009) |
| IL1B | (Liu et al., 2006) |
| IL4 | (Sfikakis, 2011) |
| IL5 | (Gharaee-Kermani et al., 1998) |
| ILK | (Li et al., 2009b) |
| INHBE | (Antsiferova and Werner, 2012) |
| ITGA2 | (Goodman and Picard, 2012; Margadant and Sonnenberg, |
| | 2010) |
| ITGA3 | (Goodman and Picard, 2012; Margadant and Sonnenberg, |
| | 2010) |
| ITGA4 | (Goodman and Picard, 2012; Margadant and Sonnenberg, |
| | 2010) |
| ITGAV | (Goodman and Picard, 2012; Margadant and Sonnenberg, |
| | 2010) |
| ITGB1 | (Goodman and Picard, 2012; Margadant and Sonnenberg, |
| | 2010) |
| ITGB3 | (Goodman and Picard, 2012; Margadant and Sonnenberg, |
| | 2010) |
| ITGB5 | (Goodman and Picard, 2012; Margadant and Sonnenberg, |
| | 2010) |
| ITGB6 | (Goodman and Picard, 2012; Margadant and Sonnenberg, |
| | 2010) |
| ITGB8 | (Goodman and Picard, 2012; Margadant and Sonnenberg, |
| | 2010) |
| JUN | (Avouac et al., 2012) |
| JUNB | (Gervasi et al., 2012) |
| LOX | (Lopez et al., 2010) |
| LOXL2 | (Barry-Hamilton et al., 2010) |
| LRP5 | (Lam et al., 2014) |
| LRP6 | (Ren et al., 2013) |
| LTBP1 | (Corchero et al., 2004) |
| MAPK1 | (Madala et al., 2012) |
| MAPK3 | (Madala et al., 2012) |
| MMP1 | (Checa et al., 2008) |
| MMP13 | (Uchinami et al., 2006) |
| MMP14 | (Zhou et al., 2004) |
| MMP2 | (Preaux et al., 1999) |
| MMP3 | (Yamashita et al., 2011) |
| MMP7 | (Greenlee et al., 2007) |
| MMP8 | (Garcia-Prieto et al., 2010) |
| MMP9 | (Wynn and Ramalingam, 2012) |
| MUC5B | (Putman et al., 2014) |
| MYC | (Madala et al., 2012) |
| NFE2L2 | (Yang et al., 2013) |
| NFKB1 | (Oakley et al., 2005) |
| PDGFA | (Bonner, 2004) |
| PDGFB | (Bonner, 2004) |
| PIK3CA | (Wynn and Ramalingam, 2012) |
| PLAT | (Ghosh and Vaughan, 2012) |
| PLAU | (Ghosh and Vaughan, 2012) |
| PLG | (Zhang et al., 2007) |
| PPARA | (Zardi et al., 2013) |
| PPARG | (Sime, 2008) |
| PTEN | (Liu et al., 2013) |
| RAC1 | (Bopp et al., 2013) |
| RHOA | (Zhou et al., 2011) |
| ROCK1 | (Zhang et al., 2006) |
| SDC4 | (Jiang et al., 2010) |
| SEMA7A | (De Minicis et al., 2013) |
| SERPINA1 | (Bartlett et al., 2009) |
| SERPINE1 | (Ghosh and Vaughan, 2012) |
| SIRT1 | (Huang et al., 2014) |
| SMAD2 | (Lan, 2011) |
| SMAD3 | (Wynn and Ramalingam, 2012) |
| SMAD4 | (Lan, 2011) |
| SMAD6 | (Lan, 2011) |
| SMAD7 | (Wynn and Ramalingam, 2012) |
| SNAI1 | (Boutet et al., 2006) |
| SOCS1 | (Yoshida et al., 2004) |
| SOCS3 | (Ogata et al., 2006) |
| SP1 | (Kum et al., 2007) |
| SPARC | (Trombetta-Esilva and Bradshaw, 2012) |
| SPHK1 | (Huang et al., 2013) |
| SPP1 | (Vetrone et al., 2009) |
| STAT1 | (Jeong et al., 2006) |
| STAT3 | (Prele et al., 2012) |
| STAT6 | (Yukawa et al., 2005) |
| TGFB1 | (Wynn and Ramalingam, 2012) |
| TGFB2 | (Wynn and Ramalingam, 2012) |
| TGFB3 | (Wynn and Ramalingam, 2012) |
| TGFBR1 | (Wynn and Ramalingam, 2012) |
| TGFBR2 | (Wynn and Ramalingam, 2012) |
| TGIF1 | (Wynn and Ramalingam, 2012) |
| TGM2 | (Olsen et al., 2011) |
| THBS1 | (Sweetwyne and Murphy-Ullrich, 2012) |
| THBS2 | (Sweetwyne and Murphy-Ullrich, 2012) |
| TIMP1 | (Selman et al., 2000) |
| TIMP2 | (Selman et al., 2000) |
| TIMP3 | (Selman et al., 2000) |
| TIMP4 | (Selman et al., 2000) |
| TNF | (Wynn and Ramalingam, 2012) |
| TP53 | (Ghosh et al., 2013) |
| VEGFA | (Kajdaniuk et al., 2011) |
| WNT1 | (Miao et al., 2013) |
| WNT2 | (Bayle et al., 2008) |
| WNT5A | (Vuga et al., 2009) |

TABLE 10
Genes in the fibrosis network. Genes were annotated by architectural cluster.

| | |
|---|---|
| Cluster 1 | AQP3, AREG, ARF4, ATP1A1, ATP2A2, BAG4, BCL2, BCR, BLK, CAV1, |
| (Green) | CAV2, CCL11, CCR2, CD300LF, CDCA5, CEBPB, CEP135, CREB1, |
| | CSF2RB, CXCL12, CXCR4, DMBT1, DNAJA1, EGF, EGFR, ELK1, |
| | FOXM1, FYN, GAB1, GAB2, ICAM1, IL10, KSR2, LAIR1, LIFR, LILRB4, |
| | MAPK1, MAPK3, MEF2C, MUC1, NTRK1, P53AIP1, PDGFRA, PDGFRB, |
| | PPARA, PRLR, PTPN1, PTPN11, PTPN2, PTPN5, PTPN6, RPS6KA1, |
| | RPS6KA2, RPS6KA3, RPS6KA4, RPS6KA5, SDC4, SEMA7A, SH2B1, |
| | SIRPA, SPN, STAT1, STAT3, THRB, TRADD, TTK, ZAP70 |
| Cluster 2 | ABTB1, ACVR1, APC, AXIN1, AXIN2, BMP7, COL5A3, CTNNB1, CXXC5, |
| (Red) | CXorf9, CYP11A1, DAB2, EIF2AK4, ENG, FBXL11, FBXL12, FOXO3, |
| | JUNB, LRP5, LRP6, MTMR10, MYBL1, MYC, MYO3A, NFE2L2, NFYA, |
| | NFYB, NFYC, PPARG, PPP6C, PSMD11, PTPN14, RAB25, RFX1, RHEBL1, |
| | RNF111, SFRP1, SKIL, SMAD1, SMAD2, SMAD3, SMAD4, SMAD5, |
| | SMAD6, SMAD7, SMAD9, SMARCA4, STRAP, TGFB2, TGFB3, TGFBR1, |
| | TGFBR2, TGIF1, TRIM28, TSSK1B, VASN, WNT1, WNT2, XPO1, ZEB2, |
| | ZNF8 |
| Cluster 3 | ANGPTL3, APP, CALR, CCL2, CCL3, CD36, CD44, CD46, CD47, CD93, |
| (Orange) | CHAD, CIB1, COL13A1, COL18A1, COL1A1, COL1A2, COL3A1, COL4A1, |
| | COL4A2, COL4A3, COL4A4, COL4A5, COL4A6, COL7A1, CTGF, CXCL6, |
| | CYR61, DCN, DPT, ELN, FCER2, FIGF, HABP2, HSPG2, IGF1, IGFBP3, |
| | IL1B, ILK, ITGA2, ITGA2B, ITGA3, ITGA4, ITGA5, ITGA6, ITGA9, |
| | ITGAV, ITGB1, ITGB3, ITGB5, ITGB6, ITGB8, JAM2, KLK3, KLK6, LOX, |
| | LTBP1, MMP1, MMP13, MMP14, MMP2, MMP3, MMP7, MMP8, MMP9, |
| | NID2, NOV, PAK1, PDGFA, PDGFB, PRELP, PRKG1, SAA1, SAA2, |
| | SERPINA1, SLC3A2, SNAI1, SPARC, SPP1, TFPI, TGFB1, TGM2, THBS1, |
| | THBS2, TIMP1, TIMP2, TIMP3, TIMP4, VEGFA |
| Cluster 4 | ACE, AKT1, ARHGAP1, BNIPL, CAMK4, CDC42, CDGAP, DEF6, |
| (Dark Blue) | DNAJA3, EDN1, EGR1, EIF4B, EIF4E, EIF4EBP1, ERF, FASLG, GAPDH, |
| | HMGB1, HMGB2, HSPA5, HSPA8, HSPA9, IFNG, IFNGR1, IFNGR2, IL5, |
| | IL13, IL13RA2, IL4, IRF2, IRF8, JUN, KTN1, MCF2L, MYL9, NDRG2, |
| | NFKB1, OPHN1, PAK2, PAK4, PDCD11, PIK3CA, PIK3CD, PIK3CG, |
| | PPP2R2A, PPP4C, PTEN, PRNP, RAC1, RAC2, RELA, RELB, RHOA, |
| | RNF34, ROCK1, RPS6KB1, RTKN, SIRT1, STAT6, TNF, TUBB, VAV3 |
| Cluster 5 | ASB1, ASB2, ASB4, ASB6, ASB7, ASB8, CFTR, CHEK2, COPS2, COPS3, |
| (Light Blue) | COPS4, COPS5, COPS6, COPS8, CUL1, CUL2, CUL3, CUL5, DNAJA2, |
| | GPS1, HIF1A, IL2RB, LOC51035, SOCS1, SOCS3, SPTLC1, TCEB1, |
| | TCEB2, TP53, VCP, WSB1, ZER1 |
| Cluster 6 | ADORA2A, ATP1A3, CYP27A1, DBH, F10, F2R, GJA1, GJA3, GRIN1, |
| (Yellow) | HAS2, HGF, HSD3B1, HSD3B2, IL1A, MUC4, NGFB, NQO2, PHOX2A, |
| | PLAT, PLA, PLG, SERPINB6, SERPINE1, SP1, SP3, SP4, SPHK1, TJP1 |

TABLE 11
Top 25 conserved miRNAs controlling the fibrosis network as ranked
by spanning score. As we previously described (Bertero et al., 2014),
spanning score considered (a) the fraction of network clusters targeted
by each miRNA and (b) the hypergeometric p-value of the overlap of
the target pool of the miRNA with the expanded fibrosis network. P-
values were normalized by the theoretical maximum p-value, defined
as the reciprocal of the number of simulations used to estimate the
distribution (in this case, 100,000 simulations were used).

| | | Genes | Clus- | | |
|---|---|---|---|---|---|
| | | Tar- | ters Tar- | p- | |
| Rank | Score | geted | geted | value | Family |
| 1 | 3.000 | 40 | 6 | 0.0001 | miR-181abcd/4262 |
| 2 | 2.875 | 35 | 6 | 0.0001 | miR-29abcd |
| 3 | 2.875 | 35 | 6 | 0.0001 | miR-27abc/27a-3p |
| 4 | 2.775 | 31 | 6 | 0.0001 | miR-144 |
| 5 | 2.725 | 29 | 6 | 0.0001 | miR-130ac/301ab/301b/ |
| | | | | | 301b-3p/454/721/4295/ |
| | | | | | 3666 |
| 6 | 2.675 | 27 | 6 | 0.0001 | miR-410/344de/344b-1-3p |
| 7 | 2.650 | 26 | 6 | 0.0001 | miR-374ab |
| 8 | 2.631 | 30 | 6 | 0.0003 | miR-128/128ab |
| 9 | 2.631 | 30 | 6 | 0.0003 | let-7/98/4458/4500 |
| 10 | 2.550 | 22 | 6 | 0.0001 | miR-221/222/222ab/1928 |
| 11 | 2.499 | 26 | 6 | 0.0004 | miR-93/93a/105/106a/291a- |
| | | | | | 3p/294/295/302abcde/372/ |
| | | | | | 373/428/519a/520be/ |
| | | | | | 520acd-3p/1378/1420ac |
| 12 | 2.476 | 33 | 6 | 0.0025 | miR-30abcdef/30abe-5p/ |
| | | | | | 384-5p |
| 13 | 2.424 | 23 | 6 | 0.0004 | miR-148ab-3p/152 |
| 14 | 2.405 | 27 | 6 | 0.0012 | miR-300/381/539-3p |
| 15 | 2.367 | 30 | 6 | 0.0034 | miR-19ab |
| 16 | 2.356 | 19 | 6 | 0.0003 | miR-199ab-5p |
| 17 | 2.327 | 30 | 6 | 0.0049 | miR-17/17-5p/20ab/20b- |
| | | | | | 5p/93/106ab/427/518a- |
| | | | | | 3p/519d |
| 18 | 2.300 | 22 | 6 | 0.0010 | miR-145 |
| 19 | 2.275 | 24 | 6 | 0.0020 | miR-101/101ab |
| 20 | 2.244 | 29 | 6 | 0.0084 | miR-590-3p |
| 21 | 2.240 | 32 | 6 | 0.0174 | miR-340-5p |
| 22 | 2.205 | 22 | 6 | 0.0024 | miR-543 |
| 23 | 2.149 | 18 | 6 | 0.0016 | miR-22/22-3p |
| 24 | 2.117 | 21 | 6 | 0.0043 | miR-202-3p |
| 25 | 2.084 | 17 | 5 | 0.0005 | miR-155 |

TABLE 12
Transcriptomic analysis of genes modulated in whole lung
from mice suffering from PH (chronic hypoxia + SU5416)
with or without inhibition of miR-130/301. Genes were
screened in order to isolate those that showed robust
modulation under hypoxic conditions (p < 0.05) and
moderate reversal of such modulation under conditions
of miR-130/301 suppression (p < 0.15).

| | | | | |
|---|---|---|---|---|
| | FOLD | | FOLD | |
| | CHANGE | P-VALUE | CHANGE | P-VALUE |
| | (HYP VS | (HYP VS | (Short-130 vs | (Short-130 vs |
| GENE | NORM) | NORM) | Short-NC) | Short-NC) |

ECM MODIFICATION/FOCAL ADHESION/FIBROSIS NETWORK

| | | | | |
|---|---|---|---|---|
| CXCL12 | 4.21 | 0.00003 | 0.47 | 0.00332 |
| VAV3 | 0.50 | 0.00050 | 1.84 | 0.00544 |
| SPP1 | 0.52 | 0.01558 | 2.10 | 0.02271 |
| ENG | 0.64 | 0.00589 | 1.44 | 0.02487 |
| HSPG2 | 1.69 | 0.00140 | 0.73 | 0.02571 |
| RPS6KA1 | 0.72 | 0.04068 | 1.36 | 0.03304 |
| VWF | 1.96 | 0.00103 | 0.75 | 0.05300 |
| ACTA2 | 1.97 | 0.01329 | 0.66 | 0.05871 |
| FLNB | 1.73 | 0.00340 | 0.75 | 0.06240 |
| MMP14 | 1.46 | 0.03474 | 0.76 | 0.06543 |
| RAP1A | 0.73 | 0.02120 | 1.28 | 0.08089 |
| COL4A4 | 1.65 | 0.00242 | 0.82 | 0.08462 |
| ADAM15 | 2.30 | 0.00065 | 0.83 | 0.08601 |
| LOXL2 | 1.65 | 0.00482 | 0.81 | 0.08870 |
| FBN1 | 1.47 | 0.00639 | 0.82 | 0.08991 |
| VAV2 | 1.61 | 0.00356 | 0.84 | 0.09386 |
| FBLN2 | 4.11 | 0.00017 | 0.69 | 0.09653 |
| HMGB2 | 0.36 | 0.00826 | 1.27 | 0.11500 |
| AQP1 | 1.77 | 0.00034 | 0.88 | 0.13183 |
| COL8A1 | 2.26 | 0.00106 | 0.78 | 0.13947 |

OTHER GENES

| | | | | |
|---|---|---|---|---|
| STXBP6 | 0.65 | 0.04062 | 1.61 | 0.00468 |
| NTRK2 | 17.04 | 0.00241 | 0.46 | 0.00486 |
| RFTN1 | 1.48 | 0.03409 | 0.60 | 0.00815 |
| AP1S2 | 0.71 | 0.02270 | 1.52 | 0.00972 |
| SPRY4 | 0.53 | 0.00938 | 1.44 | 0.01152 |
| HK2 | 0.62 | 0.04160 | 1.39 | 0.01291 |
| SCARF2 | 1.52 | 0.00662 | 0.65 | 0.01317 |
| SUCLG2 | 1.44 | 0.03114 | 0.75 | 0.01594 |
| RASGRP3 | 1.33 | 0.03034 | 0.60 | 0.01658 |
| PFKL | 1.70 | 0.01381 | 0.68 | 0.01698 |
| NOSTRIN | 1.45 | 0.01715 | 0.79 | 0.02066 |
| PER2 | 0.74 | 0.04897 | 1.72 | 0.02157 |
| IBTK | 0.81 | 0.04858 | 1.26 | 0.02220 |
| POLR3E | 1.33 | 0.03530 | 0.76 | 0.02251 |
| TMEM109 | 1.45 | 0.01995 | 0.78 | 0.02327 |
| P2RY2 | 2.08 | 0.00402 | 0.61 | 0.02370 |
| MGLL | 2.08 | 0.00110 | 0.72 | 0.02614 |
| SUMO1 | 0.76 | 0.02633 | 1.27 | 0.02824 |
| FAT4 | 1.99 | 0.00239 | 0.70 | 0.02914 |
| RET | 1.99 | 0.00095 | 0.71 | 0.02919 |
| GRIK5 | 1.35 | 0.02752 | 0.81 | 0.03845 |
| FANCE | 1.26 | 0.04985 | 0.78 | 0.03856 |
| GNAO1 | 0.60 | 0.03364 | 1.53 | 0.04349 |
| LSM11 | 1.77 | 0.00239 | 0.81 | 0.04518 |
| ETV5 | 0.76 | 0.04775 | 1.31 | 0.04656 |
| SLIT3 | 2.07 | 0.00474 | 0.79 | 0.04836 |
| SYTL2 | 1.50 | 0.01363 | 0.77 | 0.04958 |
| FBXL7 | 1.57 | 0.00287 | 0.80 | 0.05294 |
| STOM | 1.38 | 0.00786 | 0.80 | 0.05307 |
| NCALD | 1.34 | 0.01691 | 0.74 | 0.05340 |
| CKB | 1.59 | 0.00303 | 0.65 | 0.05403 |
| DUSP6 | 0.40 | 0.00157 | 1.51 | 0.05549 |
| MED16 | 1.31 | 0.04085 | 0.81 | 0.05553 |
| ETS2 | 0.80 | 0.04111 | 1.28 | 0.05941 |
| RCOR1 | 1.36 | 0.04418 | 0.78 | 0.06082 |
| FGFRL1 | 1.29 | 0.03930 | 0.82 | 0.06118 |
| CHPF | 1.40 | 0.01302 | 0.80 | 0.06142 |
| LIMK1 | 1.69 | 0.03329 | 0.69 | 0.06292 |
| TRIM65 | 1.73 | 0.00439 | 0.74 | 0.06351 |
| ELMO2 | 1.35 | 0.01703 | 0.83 | 0.06558 |
| ALDOA | 1.35 | 0.03924 | 0.76 | 0.06590 |
| ANKRD44 | 0.77 | 0.01418 | 1.23 | 0.06657 |
| MYH10 | 1.72 | 0.01490 | 0.74 | 0.06891 |
| MUS81 | 0.73 | 0.01422 | 1.21 | 0.06897 |
| DLL4 | 0.37 | 0.00019 | 1.24 | 0.07523 |
| SGK3 | 0.70 | 0.02614 | 1.24 | 0.07605 |
| N4BP3 | 2.35 | 0.00061 | 0.74 | 0.07614 |
| TEAD4 | 1.44 | 0.04813 | 0.83 | 0.07650 |
| CLSTN1 | 1.46 | 0.01228 | 0.85 | 0.07844 |
| CACNA1C | 1.51 | 0.00927 | 0.72 | 0.08000 |
| STMN1 | 0.54 | 0.00097 | 1.23 | 0.08115 |
| R3HCC1 | 1.32 | 0.03921 | 0.86 | 0.08524 |
| NTN1 | 1.60 | 0.01011 | 0.80 | 0.08636 |
| MRAS | 2.12 | 0.00367 | 0.78 | 0.08806 |
| MACF1 | 1.48 | 0.01205 | 0.81 | 0.09015 |
| GDPD5 | 1.58 | 0.01175 | 0.80 | 0.09115 |
| PARD3B | 1.43 | 0.01744 | 0.79 | 0.09220 |
| ATP6V1D | 0.75 | 0.02088 | 1.19 | 0.09542 |
| TNK2 | 1.37 | 0.01370 | 0.83 | 0.09660 |
| BLCAP | 1.32 | 0.03944 | 0.82 | 0.09677 |
| KCTD15 | 1.51 | 0.01645 | 0.79 | 0.09690 |
| PDK3 | 0.73 | 0.01232 | 1.17 | 0.09732 |
| FXYD6 | 2.83 | 0.00003 | 0.84 | 0.09775 |
| TBC1D1 | 1.31 | 0.03511 | 0.85 | 0.09809 |
| NRM | 2.04 | 0.00136 | 0.73 | 0.10018 |
| CALD1 | 1.68 | 0.00474 | 0.87 | 0.10083 |
| MAN2C1 | 1.36 | 0.02512 | 0.82 | 0.10102 |
| ATP6V1G1 | 0.80 | 0.03024 | 1.16 | 0.10203 |
| NR1H2 | 1.30 | 0.04410 | 0.84 | 0.10430 |
| ARNTL | 2.95 | 0.00766 | 0.54 | 0.10649 |
| NOTCH4 | 0.77 | 0.02985 | 1.47 | 0.10940 |
| SLC4A8 | 1.53 | 0.02325 | 0.83 | 0.11245 |
| RNPEP | 0.66 | 0.03557 | 1.31 | 0.11262 |
| JAG2 | 1.97 | 0.00269 | 0.79 | 0.11615 |
| D2HGDH | 0.69 | 0.02438 | 1.21 | 0.11896 |
| IFT80 | 0.64 | 0.04732 | 1.42 | 0.11902 |
| PLK2 | 0.43 | 0.00221 | 1.19 | 0.12078 |
| VBP1 | 0.76 | 0.01770 | 1.19 | 0.12101 |
| CIITA | 1.60 | 0.04010 | 0.59 | 0.12661 |
| HDAC9 | 2.40 | 0.00218 | 0.70 | 0.12870 |
| SNAPC4 | 1.36 | 0.02976 | 0.83 | 0.13009 |
| ATG3 | 0.79 | 0.02844 | 1.25 | 0.13045 |
| DOCK7 | 1.30 | 0.03670 | 0.87 | 0.13046 |
| IGFBP2 | 2.04 | 0.00295 | 0.58 | 0.13582 |
| ARL15 | 0.58 | 0.01068 | 1.25 | 0.13851 |
| MAST4 | 1.59 | 0.00734 | 0.85 | 0.13853 |
| THBD | 1.57 | 0.03021 | 0.84 | 0.14291 |
| PLXNC1 | 0.64 | 0.00913 | 1.17 | 0.14301 |
| RGS9 | 0.44 | 0.00058 | 1.26 | 0.14393 |
| TRPS1 | 0.70 | 0.02413 | 1.24 | 0.14545 |
| POLR2E | 1.22 | 0.04429 | 0.85 | 0.14817 |
| PHLDA3 | 1.45 | 0.01297 | 0.81 | 0.14988 |

TABLE 13
Pathway enrichment of genes modulated by miR-130/301 in whole lung of mice suffering from hypoxia +
SU5416-induced PH: Genes were screened as described above (see Table S5). Pathway enrichment
was performed as previously described (Parikh et al., 2012), and functional pathways were ranked
according to the hypergeometric p-value of their overlap with the screened gene set.

| | | GENES IN | # GENES IN | | |
|---|---|---|---|---|---|
| PATHWAY | DATABASE | PATHWAY | PATHWAY | P-VALUE | FDR |
| Extracellular matrix | Reactome | SPP1, LOXL2, | 9 | 2.0E−5 | 4.750E−02 |
| organization | | MMP14, ADAM15, | | | |
| | | COL8A1, COL4A4, | | | |
| | | FBN1, HSPG2, | | | |
| | | FBLN2 | | | |
| HIF-1-alpha | NCBI | CXCL12, ALDOA, | 5 | 5.0E−5 | 6.2E−02 |
| transcription factor | | ENG, PFKL, HK2 | | | |
| network | | | | | |
| MAPK signaling | KEGG | STMN1, MRAS, | 9 | 8.0E−5 | 5.7E−02 |
| pathway | | NTRK2, RAP1A, | | | |
| | | CACNA1C, FLNB, | | | |
| | | RASGRP3, RPS6KA1, | | | |
| | | DUSP6 | | | |
| Focal adhesion | KEGG | SPP1, VAV3, VAV2, | 7 | 0.0020 | 1.796E−01 |
| | | RAP1A, FLNB, | | | |
| | | COL4A4, VWF | | | |
| Circadian clock system | NCBI | ARNTL, PER2 | 2 | 0.0022 | 1.708E−01 |
| Fructose and mannose | KEGG | ALDOA, PFKL, HK2 | 3 | 0.0038 | 2.547E−01 |
| metabolism | | | | | |
| Signaling by NOTCH3 | Reactome | JAG2, DLL4 | 2 | 0.0041 | 2.421E−01 |
| Axon guidance | KEGG | CXCL12, PLXNC1, | 5 | 0.0048 | 2.464E−01 |
| | | NTN1, LIMK1, | | | |
| | | SLIT3 | | | |
| ECM-receptor | KEGG | SPP1, COL4A4, | 4 | 0.0066 | 2.687E−01 |
| interaction | | HSPG2, VWF | | | |
| Notch signaling | KEGG | JAG2, DLL4, | 3 | 0.0083 | 2.888E−01 |
| pathway | | NOTCH4 | | | |
| Circadian rhythm | NCBI | ARNTL, PER2 | 2 | 0.0085 | 2.737E−01 |
| pathway | | | | | |
| Carbon metabolism | KEGG | SUCLG2, ALDOA, | 4 | 0.0086 | 2.567E−01 |
| | | PFKL, HK2 | | | |
| Notch signaling | NCBI | JAG2, DLL4, | 3 | 0.0103 | 2.833E−01 |
| pathway | | NOTCH4 | | | |
| Dorso-ventral axis | KEGG | ETS2, NOTCH4 | 2 | 0.0183 | 2.872E−01 |
| formation | | | | | |
| Integrin signalling | NCBI | RAP1A, FLNB, | 5 | 0.0118 | 2.831E−01 |
| pathway | | ELMO2, COL8A1, | | | |
| | | COL4A4 | | | |
| Nicotinic acetylcholine | NCBI | MYH10, ACTA2 | 2 | 0.0156 | 3.556E−01 |
| receptor signaling | | | | | |
| pathway | | | | | |
| Alpha9 beta1 integrin | NCBI | SPP1, ADAM15 | 2 | 0.0183 | 3.892E−01 |
| signaling events | | | | | |
| Leukocyte | KEGG | CXCL12, VAV3, | 4 | 0.0190 | 3.851E−01 |
| transendothelial | | VAV2, RAP1A | | | |
| migration | | | | | |
| Glycolysis/ | KEGG | ALDOA, PFKL, | 3 | 0.0194 | 3.48E−01 |
| Gluconeogenesis | | HK2 | | | |
| Semaphorin | KEGG | PLXNC1, MYH10, | 3 | 0.0194 | 3.48E−01 |
| interactions | | LIMK1 | | | |
| Beta1 integrin cell | NCBI | SPP1, COL4A4, | 3 | 0.0194 | 3.48E−01 |
| surface interactions | | FBN1 | | | |
| Long-term potentiation | KEGG | RAP1A, CACNA1C, | 3 | 0.0202 | 3.492E−01 |
| | | RPS6KA1 | | | |
| CDC42 signaling | NCBI | VAV2, TNK2, LIMK1 | 3 | 0.0226 | 3.797E−01 |
| events | | | | | |
| Collecting duct acid | KEGG | ATP6V1D, ATP6V1G1 | 2 | 0.0228 | 3.576E−01 |
| secretion | | | | | |
| Integrin alphaIIb beta3 | Reactome | RAP1A, VWF | 2 | 0.0228 | 3.576E−01 |
| signaling | | | | | |
| B cell receptor | KEGG | VAV3, VAV2, | 3 | 0.0243 | 3.603E−01 |
| signaling pathway | | RASGRP3 | | | |
| Signaling by NOTCH1 | Reactome | JAG2, DLL4, | 3 | 0.0243 | 3.603E−01 |
| | | HDAC9 | | | |
| Pentose phosphate | KEGG | ALDOA, PFKL | 2 | 0.0244 | 3.509E−01 |
| pathway | | | | | |
| Osteopontin-mediated | NCBI | SPP1, VAV3 | 2 | 0.0260 | 3.700E−01 |
| events | | | | | |
| Nectin adhesion | NCBI | VAV2, RAP1A | 2 | 0.0277 | 3.512E−01 |
| pathway | | | | | |
| Circadian rhythm | KEGG | ARNTL, PER2 | 2 | 0.0277 | 3.512E−01 |
| Regulation of CDC42 | NCBI | VAV3, VAV2 | 2 | 0.0277 | 3.512E−01 |
| activity | | | | | |
| Galactose metabolism | KEGG | PFKL, HK2 | 2 | 0.0277 | 3.512E−01 |
| EPHA forward | NCBI | VAV3, VAV2 | 2 | 0.0294 | 3.538E−01 |
| signaling | | | | | |
| GPVI-mediated | Reactome | VAV3, VAV2 | 2 | 0.0294 | 3.538E−01 |
| activation cascade | | | | | |
| CXCR4-mediated | NCBI | CXCL12, LIMK1, | 3 | 0.0308 | 3.602E−01 |
| signaling events | | GNAO1 | | | |
| Signaling by NOTCH2 | Reactome | JAG2, DLL4 | 2 | 0.0311 | 3.401E−01 |
| RNA polymerase | KEGG | POLR2E, POLR3E | 2 | 0.0311 | 3.401E−01 |
| Formation of Fibrin | Reactome | THBD, VWF | 2 | 0.0311 | 3.401E−01 |
| Clot (Clotting Cascade) | | | | | |

### References for Example 5

1. Apostolopoulou, S. C., Manginas, A., Cokkinos, D. V., and Rammos, S. (2007). Long-term oral bosentan treatment in patients with pulmonary arterial hypertension related to congenital heart disease: a 2-year study. Heart 93, 350-354.
2. Austin, E. D., and Loyd, J. E. (2014). The genetics of pulmonary arterial hypertension. Circ Res 115, 189-202.
3. Badesch, D. B., Abman, S. H., Simonneau, G., Rubin, L. J., and McLaughlin, V. V. (2007). Medical therapy for pulmonary arterial hypertension: updated ACCP evidence-based clinical practice guidelines. Chest 131, 1917-1928.
4. Bertero, T., Cottrill, K., Krauszman, A., Lu, Y., Annis, S., Hale, A., Bhat, B., Waxman, A. B., Chau, B. N., Kueb ler, W. M., et al. (2014a). The microRNA-130/301 family controls vasoconstriction in pulmonary hypertension. J Biol Chem 290, 2069-2085.
5. Bertero, T., Lu, Y., Annis, S., Hale, A., Bhat, B., Saggar, R., Saggar, R., Wallace, W. D., Ross, D. J., Vargas, S. O., et al. (2014b). Systems-level regulation of microRNA networks by miR-130/301 promotes pulmonary hypertension. J Clin Invest 124, 3514-3528.
6. Boutet, K., Montani, D., Jais, X., Yaici, A., Sitbon, O., Simonneau, G., and Humbert, M. (2008). Therapeutic advances in pulmonary arterial hypertension. Ther Adv Respir Dis 2, 249-265.
7. Butcher, D. T., Alliston, T., and Weaver, V. M. (2009). A tense situation: forcing tumour progression. Nat Rev Cancer 9, 108-122.
8. Chan, S. Y., and Loscalzo, J. (2008a). Pathogenic mechanisms of pulmonary arterial hypertension. J Mol Cell Cardiol 44, 14-30.
9. Chan, S. Y., and Loscalzo, J. (2008b). Pathogenic mechanisms of pulmonary arterial hypertension. J Mol Cell Cardiol 44, 14-30.
10. Chaumais, M. C., Perrin, S., Sitbon, O., Simonneau, G., Humbert, M., and Montani, D. (2013). Pharmacokinetic evaluation of sildenafil as a pulmonary hypertension treatment. Ex Op Drug Metab Toxicol 9, 1193-1205.
11. Chou, J., Lin, J. H., Brenot, A., Kim, J. W., Provot, S., and Werb, Z. (2013). GATA3 suppresses metastasis and modulates the tumour microenvironment by regulating microRNA-29b expression. Nat Cell Biol 15, 201-213.
12. Ciuclan, L., Bonneau, O., Hussey, M., Duggan, N., Holmes, A. M., Good, R., Stringer, R., Jones, P., Morrell, N. W., Jarai, G., et al. (2011). A Novel Murine Model of Severe Pulmonary Arterial Hypertension. Am J Respir Crit Care Med 184, 1171-1182.
13. Cordenonsi, M., Zanconato, F., Azzolin, L., Forcato, M., Rosato, A., Frasson, C., Inui, M., Montagner, M., Parenti, A. R., Poletti, A., et al. (2011). The Hippo transducer TAZ confers cancer stem cell-related traits on breast cancer cells. Cell 147, 759-772.
14. Dupont, S., Morsut, L., Aragona, M., Enzo, E., Giulitti, S., Cordenonsi, M., Zanconato, F., Le Digabel, J., Forcato, M., Bicciato, S., et al. (2011). Role of YAP/TAZ in mechanotransduction. Nature 474, 179-183.
15. Friedman, R. C., Farh, K. K., Burge, C. B., and Bartel, D. P. (2009). Most mammalian mRNAs are conserved targets of microRNAs. Genome Res 19, 92-105.
16. Fujii, M., Toyoda, T., Nakanishi, H., Yatabe, Y., Sato, A., Matsudaira, Y., Ito, H., Murakami, H., Kondo, Y., Kondo, E., et al. (2012). TGF-beta synergizes with defects in the Hippo pathway to stimulate human malignant mesothelioma growth. J Exp Med 209, 479-494.
17. Galie, N., Beghetti, M., Gatzoulis, M. A., Granton, J., Berger, R. M., Lauer, A., Chiossi, E., and Landzberg, M. (2006). Bosentan therapy in patients with Eisenmenger syndrome: a multicenter, double-blind, randomized, placebo-controlled study. Circulation 114, 48-54.
18. Gan, C. T., Holverda, S., Marcus, J. T., Paulus, W. J., Marques, K. M., Bronzwaer, J. G., Twisk, J. W., Boonstra, A., Postmus, P. E., and Vonk-Noordegraaf, A. (2007a). Right ventricular diastolic dysfunction and the acute effects of sildenafil in pulmonary hypertension patients. Chest 132, 11-17.
19. Gan, C. T., Lankhaar, J. W., Westerhof, N., Marcus, J. T., Becker, A., Twisk, J. W., Boonstra, A., Postmus, P. E., and Vonk-Noordegraaf, A. (2007b). Noninvasively assessed pulmonary artery stiffness predicts mortality in pulmonary arterial hypertension. Chest 132, 1906-1912.
20. Germain, M., Eyries, M., Montani, D., Poirier, O., Girerd, B., Dorfmuller, P., Coulet, F., Nadaud, S., Maugenre, S., Guignabert, C., et al. (2013). Genome-wide association analysis identifies a susceptibility locus for pulmonary arterial hypertension. Nat Genet 45, 518-521.
21. Hansmann, G., de Jesus Perez, V. A., Alastalo, T. P., Alvira, C. M., Guignabert, C., Bekker, J. M., Schellong, S., Urashima, T., Wang, L., Morrell, N. W., et al. (2008). An antiproliferative BMP-2/PPARgamma/apoE axis in human and murine SMCs and its role in pulmonary hypertension. J Clin Invest 118, 1846-1857.
22. Harrison, R. E., Flanagan, J. A., Sankelo, M., Abdalla, S. A., Rowell, J., Machado, R. D., Elliott, C. G., Robbins, I. M., Olschewski, H., McLaughlin, V., et al. (2003). Molecular and functional analysis identifies ALK-1 as the predominant cause of pulmonary hypertension related to hereditary haemorrhagic telangiectasia. J Med Genet 40, 865-871.
23. Harvey, K. F., Zhang, X., and Thomas, D. M. (2013). The Hippo pathway and human cancer. Nat Rev Cancer 13, 246-257.
24. Im, S. S., and Osborne, T. F. (2011). Liver x receptors in atherosclerosis and inflammation. Circ Res 108, 996-1001.
25. Kajstura, J., Rota, M., Hall, S. R., Hosoda, T., D'Amario, D., Sanada, F., Zheng, H., Ogorek, B., Rondon-Clavo, C., Ferreira-Martins, J., et al. (2011). Evidence for human lung stem cells. N Engl J Med 364, 1795-1806.
26. Kim, K. M., Choi, Y. J., Hwang, J. H., Kim, A. R., Cho, H. J., Hwang, E. S., Park, J. Y., Lee, S. H., and Hong, J. H. (2014). Shear stress induced by an interstitial level of slow flow increases the osteogenic differentiation of mesenchymal stem cells through TAZ activation. PLoS One 9, e92427.
27. Kothapalli, D., Liu, S. L., Bae, Y. H., Monslow, J., Xu, T., Hawthorne, E. A., Byfield, F. J., Castagnino, P., Rao, S., Rader, D. J., et al. (2012). Cardiovascular protection by ApoE and ApoE-HDL linked to suppression of ECM gene expression and arterial stiffening. Cell Rep 2, 1259-1271.
28. Lammers, S., Scott, D., Hunter, K., Tan, W., Shandas, R., and Stenmark, K. R. (2012). Mechanics and Function of the Pulmonary Vasculature: Implications for Pulmonary Vascular Disease and Right Ventricular Function. Compr Physiol 2, 295-319.
29. Levental, K. R., Yu, H., Kass, L., Lakins, J. N., Egeblad, M., Erler, J. T., Fong, S. F., Csiszar, K., Giaccia, A., Weninger, W., et al. (2009). Matrix crosslinking forces tumor progression by enhancing integrin signaling. Cell 139, 891-906.
30. Lian, I., Kim, J., Okazawa, H., Zhao, J., Zhao, B., Yu, J., Chinnaiyan, A., Israel, M. A., Goldstein, L. S., Abujarour, R., et al. (2010). The role of YAP transcription coactivator in regulating stem cell self-renewal and differentiation. Genes Dev 24, 1106-1118.
31. Liu, F., Lagares, D., Choi, K. M., Stopfer, L., Marinkovic, A., Vrbanac, V., Probst, C. K., Hiemer, S. E., Sisson, T. H., Horowitz, J. C., et al. (2015). Mechanosignaling through YAP and TAZ drives fibroblast activation and fibrosis. Am J Physiol Lung Cell Mol Physiol 308, L344-357.
32. Lu, P., Weaver, V. M., and Werb, Z. (2012). The extracellular matrix: a dynamic niche in cancer progression. J Cell Biol 196, 395-406.
33. Mahapatra, S., Nishimura, R. A., Sorajja, P., Cha, S., and McGoon, M. D. (2006). Relationship of pulmonary arterial capacitance and mortality in idiopathic pulmonary arterial hypertension. J Am Coll Cardiol 47, 799-803.
34. Maki, J. M., Sormunen, R., Lippo, S., Kaarteenaho-Wiik, R., Soininen, R., and Myllyharju, J. (2005). Lysyl oxidase is essential for normal development and function of the respiratory system and for the integrity of elastic and collagen fibers in various tissues. Am J Pathol 167, 927-936.
35. McLaughlin, V. V., Sitbon, O., Badesch, D. B., Barst, R. J., Black, C., Galie, N., Rainisio, M., Simonneau, G., and Rubin, L. J. (2005). Survival with first-line bosentan in patients with primary pulmonary hypertension. Eur Respir J 25, 244-249.
36. Mouw, J. K., Yui, Y., Damiano, L., Bainer, R. O., Lakins, J. N., Acerbi, I., Ou, G., Wijekoon, A. C., Levental, K. R., Gilbert, P. M., et al. (2014). Tissue mechanics modulate microRNA-dependent PTEN expression to regulate malignant progression. Nat Med 20, 360-367.
37. Nave, A. H., Mizikova, I., Niess, G., Steenbock, H., Reichenberger, F., Talavera, M. L., Veit, F., Herold, S., Mayer, K., Vadasz, I., et al. (2014). Lysyl oxidases play a causal role in vascular remodeling in clinical and experimental pulmonary arterial hypertension. Arterio Thromb Vasc Biol 34, 1446-1458.
38. Obad, S., dos Santos, C. O., Petri, A., Heidenblad, M., Broom, O., Ruse, C., Fu, C., Lindow, M., Stenvang, J., Straarup, E. M., et al. (2011). Silencing of microRNA families by seed-targeting tiny LNAs. Nat Genet 43, 371-378.
39. Pan, D. (2010). The hippo signaling pathway in development and cancer. Dev Cell 19, 491-505.
40. Pencheva, N., Buss, C. G., Posada, J., Merghoub, T., and Tavazoie, S. F. (2014). Broad-spectrum therapeutic suppression of metastatic melanoma through nuclear hormone receptor activation. Cell 156, 986-1001.
41. Pencheva, N., Tran, H., Buss, C., Huh, D., Drobnjak, M., Busam, K., and Tavazoie, S. F. (2012). Convergent multi-miRNA targeting of ApoE drives LRP1/LRP8-dependent melanoma metastasis and angiogenesis. Cell 151, 1068-1082.
42. Rodriguez, C., Rodriguez-Sinovas, A., and Martinez-Gonzalez, J. (2008). Lysyl oxidase as a potential therapeutic target. Drug News Perspect 21, 218-224.
43. Schafer, M., and Werner, S. (2008). Cancer as an overhealing wound: an old hypothesis revisited. Nat Rev Mol Cell Biol 9, 628-638.
44. Stenmark, K. R., and Rabinovitch, M. (2010). Emerging therapies for the treatment of pulmonary hypertension. Pediatr Crit Care Med 11, S85-90.
45. Tang, Y., Rowe, R. G., Botvinick, E. L., Kurup, A., Putnam, A. J., Seiki, M., Weaver, V. M., Keller, E. T., Goldstein, S., Dai, J., et al. (2013). MT1-MMP-dependent control of skeletal stem cell commitment via a beta1-integrin/YAP/TAZ signaling axis. Dev Cell 25, 402-416.
46. Valastyan, S., and Weinberg, R. A. (2011). Roles for microRNAs in the regulation of cell adhesion molecules. J Cell Sci 124, 999-1006.
47. van Dongen, S., Abreu-Goodger, C., and Enright, A. J. (2008). Detecting microRNA binding and siRNA off-target effects from expression data. Nat Methods 5, 1023-1025.
48. Wang, Z., and Chesler, N. C. (2011). Pulmonary vascular wall stiffness: An important contributor to the increased right ventricular afterload with pulmonary hypertension. Pulm Circ 1, 212-223.
49. Wei, J., Bhattacharyya, S., Jain, M., and Varga, J. (2012). Regulation of Matrix Remodeling by Peroxisome Proliferator-Activated Receptor-gamma: A Novel Link Between Metabolism and Fibrogenesis. Open Rheumatol J 6, 103-115.
50. Wilmarth, K. R., and Froines, J. R. (1992). In vitro and in vivo inhibition of lysyl oxidase by aminopropionitriles. J Toxicol Environ Health 37, 411-423.
51. Zhou, Z., Hu, T., Xu, Z., Lin, Z., Zhang, Z., Feng, T., Zhu, L., Rong, Y., Shen, H., Luk, J. M., et al. (2014). Targeting Hippo pathway by specific interruption of YAP-TEAD interaction using cyclic YAP-like peptides. FASEB J.
52. Antsiferova, M., and Werner, S. (2012). The bright and the dark sides of activin in wound healing and cancer. J Cell Sci 125, 3929-3937.
53. Aragona, M., Panciera, T., Manfrin, A., Giulitti, S., Michielin, F., Elvassore, N., Dupont, S., and Piccolo, S. (2013). A mechanical checkpoint controls multicellular growth through YAP/TAZ regulation by actin-processing factors. Cell 154, 1047-1059.
54. Avouac, J., Palumbo, K., Tomcik, M., Zerr, P., Dees, C., Horn, A., Maurer, B., Akhmetshina, A., Beyer, C., Sadowski, A., et al. (2012). Inhibition of activator protein 1 signaling abrogates transforming growth factor beta-mediated activation of fibroblasts and prevents experimental fibrosis. Arthritis Rheum 64, 1642-1652.
55. Baghy, K., Derso, K., Laszlo, V., Fullar, A., Peterfia, B., Paku, S., Nagy, P., Schaff, Z., Iozzo, R. V., and Kovalszky, I. (2011). Ablation of the decorin gene enhances experimental hepatic fibrosis and impairs hepatic healing in mice. Lab Invest 91, 439-451.
56. Barry-Hamilton, V., Spangler, R., Marshall, D., McCauley, S., Rodriguez, H. M., Oyasu, M., Mikels, A., Vaysberg, M., Ghermazien, H., Wai, C., et al. (2010). Allosteric inhibition of lysyl oxidase-like-2 impedes the development of a pathologic microenvironment. Nat Med 16, 1009-1017.
57. Bartlett, J. R., Friedman, K. J., Ling, S. C., Pace, R. G., Bell, S. C., Bourke, B., Castaldo, G., Castellani, C., Cipolli, M., Colombo, C., et al. (2009). Genetic modifiers of liver disease in cystic fibrosis. JAMA 302, 1076-1083.
58. Bayle, J., Fitch, J., Jacobsen, K., Kumar, R., Lafyatis, R., and Lemaire, R. (2008). Increased expression of Wnt2 and SFRP4 in Tsk mouse skin: role of Wnt signaling in altered dermal fibrillin deposition and systemic sclerosis. J Invest Dermatol 128, 871-881.
59. Bedford, J. J., Leader, J. P., and Walker, R. J. (2003). Aquaporin expression in normal human kidney and in renal disease. J Am Soc Nephrol 14, 2581-2587.
60. Bertero, T., Lu, Y., Annis, S., Hale, A., Bhat, B., Saggar, R., Saggar, R., Wallace, W. D., Ross, D. J., Vargas, S. O., et al. (2014). Systems-level regulation of microRNA networks by miR-130/301 promotes pulmonary hypertension. J Clin Invest 124, 3514-3528.
61. Besnard, A. G., Struyf, S., Guabiraba, R., Fauconnier, L., Rouxel, N., Proost, P., Uyttenhove, C., Van Snick, J., Couillin, I., and Ryffel, B. (2013). CXCL6 antibody neutralization prevents lung inflammation and fibrosis in mice in the bleomycin model. J Leukoc Biol 94, 1317-1323.
62. Blondel, V. D., Guillaume, J. L., Hendrickx, J. M., de Kerchove, C., and Lambiotte, R. (2008). Local leaders in random networks. Phys Rev E Stat Nonlin Soft Matter Phys 77, 036114.
63. Bonilla, N., Barget, N., Andrieu, M., Roulot, D., Letoumelin, P., Grando, V., Trinchet, J. C., Ganne-Carrie, N., Beaugrand, M., Deny, P., et al. (2006). Interferon gamma-secreting HCV-specific CD8+ T cells in the liver of patients with chronic C hepatitis: relation to liver fibrosis—ANRS HC EPO7 study. J Viral Hepat 13, 474-481.
64. Bonner, J. C. (2004). Regulation of PDGF and its receptors in fibrotic diseases. Cytokine Growth Factor Rev 15, 255-273.
65. Boon, M. R., van der Horst, G., van der Pluijm, G., Tamsma, J. T., Smit, J. W., and Rensen, P. C. (2011). Bone morphogenetic protein 7: a broad-spectrum growth factor with multiple target therapeutic potency. Cytokine Growth Factor Rev 22, 221-229.
66. Bopp, A., Wartlick, F., Henninger, C., Kaina, B., and Fritz, G. (2013). Rac 1 modulates acute and subacute genotoxin-induced hepatic stress responses, fibrosis and liver aging. Cell Death Dis 4, e558.
67. Boutet, A., De Frutos, C. A., Maxwell, P. H., Mayol, M. J., Romero, J., and Nieto, M. A. (2006). Snail activation disrupts tissue homeostasis and induces fibrosis in the adult kidney. EMBO J 25, 5603-5613.
68. Brilla, C. G., Reams, G. P., Maisch, B., and Weber, K. T. (1993). Renin-angiotensin system and myocardial fibrosis in hypertension: regulation of the myocardial collagen matrix. Eur Heart J 14 Suppl J, 57-61.
69. Chakraborty, S., Chopra, P., Hak, A., Dastidar, S. G., and Ray, A. (2013). Hepatocyte growth factor is an attractive target for the treatment of pulmonary fibrosis. Expert Opin Investig Drugs 22, 499-515.
70. Chan, S. Y., Zhang, Y. Y., Hemann, C., Mahoney, C. E., Zweier, J. L., and Loscalzo, J. (2009). MicroRNA-210 controls mitochondrial metabolism during hypoxia by repressing the iron-sulfur cluster assembly proteins ISCU1/2. Cell Metab 10, 273-284.
71. Checa, M., Ruiz, V., Montano, M., Velazquez-Cruz, R., Selman, M., and Pardo, A. (2008). MMP-1 polymorphisms and the risk of idiopathic pulmonary fibrosis. Hum Genet 124, 465-472.
72. Corchero, J., Martin-Partido, G., Dallas, S. L., and Fernandez-Salguero, P. M. (2004). Liver portal fibrosis in dioxin receptor-null mice that overexpress the latent transforming growth factor-beta-binding protein-1. Int J Exp Pathol 85, 295-302.
73. de Almeida, C. J., Jasmin, J. F., Del Galdo, F., and Lisanti, M. P. (2013). Genetic ablation of caveolin-2 sensitizes mice to bleomycin-induced injury. Cell Cycle 12, 2248-2254.
74. De Minicis, S., Rychlicki, C., Agostinelli, L., Saccomanno, S., Trozzi, L., Candelaresi, C., Bataller, R., Millan, C., Brenner, D. A., Vivarelli, M., et al. (2013). Semaphorin 7A contributes to TGF-beta-mediated liver fibrogenesis. Am J Pathol 183, 820-830.
75. Della Latta, V., Cabiati, M., Rocchiccioli, S., Del Ry, S., and Morales, M. A. (2013). The role of the adenosinergic system in lung fibrosis. Pharmacol Res 76, 182-189.
76. Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
77. FastQC http://www.bioinformatics.babraham.ac.uk/projects/fastqc/.
78. Fern, R. J., Yesko, C. M., Thornhill, B. A., Kim, H. S., Smithies, O., and Chevalier, R. L. (1999). Reduced angiotensinogen expression attenuates renal interstitial fibrosis in obstructive nephropathy in mice. J Clin Invest 103, 39-46.
79. Friedman, R. C., Farh, K. K., Burge, C. B., and Bartel, D. P. (2009). Most mammalian mRNAs are conserved targets of microRNAs. Genome Res 19, 92-105.
80. Gao, X., Wang, G., Zhang, W., Peng, Q., Xue, M., and Jinhong, H. (2013). Expression of pulmonary aquaporin 1 is dramatically upregulated in mice with pulmonary fibrosis induced by bleomycin. Arch Med Sci 9, 916-921.
81. Garcia-Prieto, E., Gonzalez-Lopez, A., Cabrera, S., Astudillo, A., Gutierrez-Fernandez, A., Fanjul-Fernandez, M., Batalla-Solis, E., Puente, X. S., Fueyo, A., Lopez-Otin, C., et al. (2010). Resistance to bleomycin-induced lung fibrosis in MMP-8 deficient mice is mediated by interleukin-10. PLoS One 5, e13242.
82. Gervasi, M., Bianchi-Smiraglia, A., Cummings, M., Zheng, Q., Wang, D., Liu, S., and Bakin, A. V. (2012). JunB contributes to Id2 repression and the epithelial-mesenchymal transition in response to transforming growth factor-beta. J Cell Biol 196, 589-603.
83. Gharaee-Kermani, M., McGarry, B., Lukacs, N., Huffnagle, G., Egan, R. W., and Phan, S. H. (1998). The role of IL-5 in bleomycin-induced pulmonary fibrosis. J Leukoc Biol 64, 657-666.
84. Ghosh, A. K., Quaggin, S. E., and Vaughan, D. E. (2013). Molecular basis of organ fibrosis: potential therapeutic approaches. Exp Biol Med (Maywood) 238, 461-481.
85. Ghosh, A. K., and Vaughan, D. E. (2012). PAI-1 in tissue fibrosis. J Cell Physiol 227, 493-507.
86. Gieling, R. G., Wallace, K., and Han, Y. P. (2009). Interleukin-1 participates in the progression from liver injury to fibrosis. Am J Physiol Gastrointest Liver Physiol 296, G1324-1331.
87. Goodman, S. L., and Picard, M. (2012). Integrins as therapeutic targets. Trends Pharmacol Sci 33, 405-412.
88. Graham, B. B., Chabon, J., Gebreab, L., Poole, J., Debella, E., Davis, L., Tanaka, T., Sanders, L., Dropcho, N., Bandeira, A., et al. (2013a). Transforming growth factor-beta signaling promotes pulmonary hypertension caused by Schistosoma mansoni. Circulation 128, 1354-1364.
89. Graham, B. B., Chabon, J., Kumar, R., Kolosionek, E., Gebreab, L., Debella, E., Edwards, M., Diener, K., Shade, T., Bifeng, G., et al. (2013b). Protective role of IL-6 in vascular remodeling in Schistosoma pulmonary hypertension. Am J Respir Cell Mol Biol 49, 951-959.
90. Granel, B., Chevillard, C., and Dessein, A. (2007). [Interleukin 13 and interleukin 13 receptor involvement in systemic sclerosis]. Rev Med Interne 28, 613-622.
91. Greenlee, K. J., Werb, Z., and Kheradmand, F. (2007). Matrix metalloproteinases in lung: multiple, multifarious, and multifaceted. Physiol Rev 87, 69-98.
92. Grygielko, E. T., Martin, W. M., Tweed, C., Thornton, P., Harling, J., Brooks, D. P., and Laping, N. J. (2005). Inhibition of gene markers of fibrosis with a novel inhibitor of transforming growth factor-beta type I receptor kinase in puromycin-induced nephritis. J Pharmacol Exp Ther 313, 943-951.
93. Guo, Y., Xiao, L., Sun, L., and Liu, F. (2012). Wnt/beta-catenin signaling: a promising new target for fibrosis diseases. Physiol Res 61, 337-346.
94. Gvaramia, D., Blaauboer, M. E., Hanemaaijer, R., and Everts, V. (2013). Role of caveolin-1 in fibrotic diseases. Matrix Biol 32, 307-315.
95. Hansmann, G., Wagner, R., Schellong, S., Perez, V., Urashima, T., Wang, L., Sheikh, A., Suen, R., Stewart, D., and Rabinovitch, M. (2007). Pulmonary arterial hypertension is linked to insulin resistance and reversed by peroxisome proliferator-activated receptor-gamma activation. Circulation 115, 1275-1284.
96. Hu, B., Castillo, E., Harewood, L., Ostano, P., Reymond, A., Dummer, R., Raffoul, W., Hoetzenecker, W., Hofbauer, G. F., and Dotto, G. P. Multifocal epithelial tumors and field cancerization from loss of mesenchymal CSL signaling. Cell 149, 1207-1220.
97. Hu, B., Wu, Z., Nakashima, T., and Phan, S. H. (2012). Mesenchymal-specific deletion of C/EBPbeta suppresses pulmonary fibrosis. Am J Pathol 180, 2257-2267.
98. Huang, L. S., Berdyshev, E., Mathew, B., Fu, P., Gorshkova, I. A., He, D., Ma, W., Noth, I., Ma, S. F., Pendyala, S., et al. (2013). Targeting sphingosine kinase 1 attenuates bleomycin-induced pulmonary fibrosis. FASEB J 27, 1749-1760.
99. Huang, X. Z., Wen, D., Zhang, M., Xie, Q., Ma, L., Guan, Y., Ren, Y., Chen, J., and Hao, C. M. (2014). Sirt1 activation ameliorates renal fibrosis by inhibiting the TGF-beta/Smad3 pathway. J Cell Biochem 115, 996-1005.
100. Humphreys, B. D., Xu, F., Sabbisetti, V., Grgic, I., Naini, S. M., Wang, N., Chen, G., Xiao, S., Patel, D., Henderson, J. M., et al. (2013). Chronic epithelial kidney injury molecule-1 expression causes murine kidney fibrosis. J Clin Invest 123, 4023-4035.
101. Jeong, W. I., Park, O., Radaeva, S., and Gao, B. (2006). STAT1 inhibits liver fibrosis in mice by inhibiting stellate cell proliferation and stimulating NK cell cytotoxicity. Hepatology 44, 1441-1451.
102. Jiang, D., Liang, J., Campanella, G. S., Guo, R., Yu, S., Xie, T., Liu, N., Jung, Y., Homer, R., Meltzer, E. B., et al. (2010). Inhibition of pulmonary fibrosis in mice by CXCL10 requires glycosaminoglycan binding and syndecan-4. J Clin Invest 120, 2049-2057.
103. Kajdaniuk, D., Marek, B., Borgiel-Marek, H., and Kos-Kudla, B. (2011). Vascular endothelial growth factor (VEGF)—part 1: in physiology and pathophysiology. Endokrynol Pol 62, 444-455.
104. Kim, W., Moon, S. O., Lee, S. Y., Jang, K. Y., Cho, C. H., Koh, G. Y., Choi, K. S., Yoon, K. H., Sung, M. J., Kim, D. H., et al. (2006). COMP-angiopoietin-1 ameliorates renal fibrosis in a unilateral ureteral obstruction model. J Am Soc Nephrol 17, 2474-2483.
105. Kisseleva, T., and Brenner, D. A. (2011). Anti-fibrogenic strategies and the regression of fibrosis. Best Pract Res Clin Gastroenterol 25, 305-317.
106. Kum, Y. S., Kim, K. H., Park, T. I., Suh, I. S., Oh, H. K., Cho, C. H., Park, J. B., Chang, Y. C., Park, J. H., Lee, K. G., et al. (2007). Antifibrotic effect via the regulation of transcription factor Spl in lung fibrosis. Biochem Biophys Res Commun 363, 368-374.
107. Lai, C. F., Lin, S. L., Chiang, W. C., Chen, Y. M., Wu, V. C., Young, G. H., Ko, W. J., Kuo, M. L., Tsai, T. J., and Wu, K. D. (2014). Blockade of Cysteine-rich Protein 61 Attenuates Renal Inflammation and Fibrosis after Ischemic Kidney Injury. Am J Physiol Renal Physiol.
108. Lam, A. P., Herazo-Maya, J. D., Sennello, J. A., Flozak, A. S., Russell, S., Mutlu, G. M., Budinger, G. R., DasGupta, R., Varga, J., Kaminski, N., et al. (2014). Wnt coreceptor lrp5 is a driver of idiopathic pulmonary fibrosis. Am J Respir Crit Care Med 190, 185-195.
109. Lan, H. Y. (2011). Diverse roles of TGF-beta/Smads in renal fibrosis and inflammation. Int J Biol Sci 7, 1056-1067.
110. Law, C. W., Chen, Y., Shi, W., and Smyth, G. K. (2014). voom: precision weights unlock linear model analysis tools for RNA-seq read counts. Genome Biol 15, R29.
111. Li, H., Handsaker, B., Wysoker, A., Fennell, T., Ruan, J., Homer, N., Marth, G., Abecasis, G., and Durbin, R. (2009a). The Sequence Alignment/Map format and SAMtools. Bioinformatics 25, 2078-2079.
112. Li, Y., Tan, X., Dai, C., Stolz, D. B., Wang, D., and Liu, Y. (2009b). Inhibition of integrin-linked kinase attenuates renal interstitial fibrosis. J Am Soc Nephrol 20, 1907-1918.
113. Liedtke, C. M. (1992). Electrolyte transport in the epithelium of pulmonary segments of normal and cystic fibrosis lung. FASEB J 6, 3076-3084.
114. Liu, S., Parapuram, S. K., and Leask, A. (2013). Fibrosis caused by loss of PTEN expression in mouse fibroblasts is crucially dependent on CCN2. Arthritis Rheum 65, 2940-2944.
115. Liu, W., Ding, I., Chen, K., Olschowka, J., Xu, J., Hu, D., Morrow, G. R., and Okunieff, P. (2006). Interleukin 1beta (IL1B) signaling is a critical component of radiation-induced skin fibrosis. Radiat Res 165, 181-191.
116. Lopez, B., Gonzalez, A., Hermida, N., Valencia, F., de Teresa, E., and Diez, J. (2010). Role of lysyl oxidase in myocardial fibrosis: from basic science to clinical aspects. Am J Physiol Heart Circ Physiol 299, H1-9.
117. Madala, S. K., Schmidt, S., Davidson, C., Ikegami, M., Wert, S., and Hardie, W. D. (2012). MEK-ERK pathway modulation ameliorates pulmonary fibrosis associated with epidermal growth factor receptor activation. Am J Respir Cell Mol Biol 46, 380-388.
118. Maher, T. M. (2012). Idiopathic pulmonary fibrosis: pathobiology of novel approaches to treatment. Clin Chest Med 33, 69-83.
119. Mandal, P., Pritchard, M. T., and Nagy, L. E. (2010). Anti-inflammatory pathways and alcoholic liver disease: role of an adiponectin/interleukin-10/heme oxygenase-1 pathway. World J Gastroenterol 16, 1330-1336.
120. Mangieri, D., Corradi, D., Martorana, D., Malerba, G., Palmisano, A., Libri, I., Bartoli, V., Carnevali, M. L., Goldoni, M., Govoni, P., et al. (2012). Eotaxin/CCL11 in idiopathic retroperitoneal fibrosis. Nephrol Dial Transplant 27, 3875-3884.
121. Margadant, C., and Sonnenberg, A. (2010). Integrin-TGF-beta crosstalk in fibrosis, cancer and wound healing. EMBO Rep 11, 97-105.
122. Maring, J. A., Trojanowska, M., and ten Dijke, P. (2012). Role of endoglin in fibrosis and scleroderma. Int Rev Cell Mol Biol 297, 295-308.
123. Miao, C. G., Yang, Y. Y., He, X., Huang, C., Huang, Y., Zhang, L., Lv, X. W., Jin, Y., and Li, J. (2013). Wnt signaling in liver fibrosis: progress, challenges and potential directions. Biochimie 95, 2326-2335.
124. Murphy, M., McMahon, R., Lappin, D. W., and Brady, H. R. (2002). Gremlins: is this what renal fibrogenesis has come to? Exp Nephrol 10, 241-244.
125. Oakley, F., Mann, J., Nailard, S., Smart, D. E., Mungalsingh, N., Constandinou, C., Ali, S., Wilson, S. J., Millward-Sadler, H., Iredale, J. P., et al. (2005). Nuclear factor-kappaB 1 (p50) limits the inflammatory and fibrogenic responses to chronic injury. Am J Pathol 166, 695-708.
126. Ogata, H., Chinen, T., Yoshida, T., Kinjyo, I., Takaesu, G., Shiraishi, H., Iida, M., Kobayashi, T., and Yoshimura, A. (2006). Loss of SOCS3 in the liver promotes fibrosis by enhancing STAT3-mediated TGF-beta1 production. Oncogene 25, 2520-2530.
127. Olsen, K. C., Sapinoro, R. E., Kottmann, R. M., Kulkarni, A. A., Iismaa, S. E., Johnson, G. V., Thatcher, T. H., Phipps, R. P., and Sime, P. J. (2011). Transglutaminase 2 and its role in pulmonary fibrosis. Am J Respir Crit Care Med 184, 699-707.
128. Parikh, V. N., Jin, R. C., Rabello, S., Gulbahce, N., White, K., Hale, A., Cottrill, K. A., Shaik, R. S., Waxman, A. B., Zhang, Y. Y., et al. (2012). MicroRNA-21 Integrates Pathogenic Signaling to Control Pulmonary Hypertension: Results of a Network Bioinformatics Approach. Circulation 125, 1520-1532.
129. Pencheva, N., Buss, C. G., Posada, J., Merghoub, T., and Tavazoie, S. F. (2014). Broad-spectrum therapeutic suppression of metastatic melanoma through nuclear hormone receptor activation. Cell 156, 986-1001.
130. Preaux, A. M., Mallat, A., Nhieu, J. T., D'Ortho, M. P., Hembry, R. M., and Mavier, P. (1999). Matrix metalloproteinase-2 activation in human hepatic fibrosis regulation by cell-matrix interactions. Hepatology 30, 944-950.
131. Prele, C. M., Yao, E., O'Donoghue, R. J., Mutsaers, S. E., and Knight, D. A. (2012). STAT3: a central mediator of pulmonary fibrosis? Proc Am Thorac Soc 9, 177-182.
132. Putman, R. K., Rosas, I. O., and Hunninghake, G. M. (2014). Genetics and early detection in idiopathic pulmonary fibrosis. Am J Respir Crit Care Med 189, 770-778.
133. Reddy, V., Meyrick, B., Wong, J., Khoor, A., Liddicoat, J., Hanley, F., and Fineman, J. (1995). In utero placement of aortopulmonary shunts. A model of postnatal pulmonary hypertension with increased pulmonary blood flow in lambs. Circulation 92, 606-613.
134. Reichenbach, V., Ros, J., Fernandez-Varo, G., Casals, G., Melgar-Lesmes, P., Campos, T., Makriyannis, A., Morales-Ruiz, M., and Jimenez, W. (2012). Prevention of fibrosis progression in CC14-treated rats: role of the hepatic endocannabinoid and apelin systems. J Pharmacol Exp Ther 340, 629-637.
135. Ren, S., Johnson, B. G., Kida, Y., Ip, C., Davidson, K. C., Lin, S. L., Kobayashi, A., Lang, R. A., Hadjantonakis, A. K., Moon, R. T., et al. (2013). LRP-6 is a coreceptor for multiple fibrogenic signaling pathways in pericytes and myofibroblasts that are inhibited by DKK-1. Proc Natl Acad Sci USA 110, 1440-1445.
136. Safaeian, L., Abed, A., and Vaseghi, G. (2014). The role of Bcl-2 family proteins in pulmonary fibrosis. Eur J Pharmacol 741, 281-289.
137. Selman, M., Ruiz, V., Cabrera, S., Segura, L., Ramirez, R., Barrios, R., and Pardo, A. (2000). TIMP-1, -2, -3, and -4 in idiopathic pulmonary fibrosis. A prevailing nondegradative lung microenvironment? Am J Physiol Lung Cell Mol Physiol 279, L562-574.
138. Sfikakis, P. P. (2011). Is IL-4 a potential therapeutic target in systemic sclerosis-associated pulmonary fibrosis? Clin Immunol 141, 240-241.
139. Shi, W., Bellusci, S., and Warburton, D. (2007). Lung development and adult lung diseases. Chest 132, 651-656.
140. Sime, P. J. (2008). The antifibrogenic potential of PPARgamma ligands in pulmonary fibrosis. J Invest Med 56, 534-538.
141. Smyth, G. K., Michaud, J., and Scott, H. S. (2005). Use of within-array replicate spots for assessing differential expression in microarray experiments. Bioinformatics 21, 2067-2075.
142. Sokolovic, A., Rodriguez-Ortigosa, C. M., Bloemendaal, L. T., Oude Elferink, R. P., Prieto, J., and Bosma, P. J. (2013). Insulin-like growth factor 1 enhances bile-duct proliferation and fibrosis in Abcb4(−/−) mice. Biochim Biophys Acta 1832, 697-704.
143. Steiner, M. K., Syrkina, O. L., Kolliputi, N., Mark, E. J., Hales, C. A., and Waxman, A. B. (2009). Interleukin-6 overexpression induces pulmonary hypertension. Circ Res 104, 236-244, 228p following 244.
144. Sweetwyne, M. T., and Murphy-Ullrich, J. E. (2012). Thrombospondinl in tissue repair and fibrosis: TGF-beta-dependent and independent mechanisms. Matrix Biol 31, 178-186.
145. Swigris, J. J., and Brown, K. K. (2010). The role of endothelin-1 in the pathogenesis of idiopathic pulmonary fibrosis. BioDrugs 24, 49-54.
146. Trombetta-Esilva, J., and Bradshaw, A. D. (2012). The Function of SPARC as a Mediator of Fibrosis. Open Rheumatol J 6, 146-155.
147. Tsutsumi, H., Tanaka, T., Ohashi, N., Masuno, H., Tamamura, H., Hiramatsu, K., Araki, T., Ueda, S., Oishi, S., and Fujii, N. (2007). Therapeutic potential of the chemokine receptor CXCR4 antagonists as multifunctional agents. Biopolymers 88, 279-289.
148. Uchinami, H., Seki, E., Brenner, D. A., and D'Armiento, J. (2006). Loss of MMP 13 attenuates murine hepatic injury and fibrosis during cholestasis. Hepatology 44, 420-429.
149. van Dongen, S., Abreu-Goodger, C., and Enright, A. J. (2008). Detecting microRNA binding and siRNA off-target effects from expression data. Nat Methods 5, 1023-1025.
150. Vetrone, S. A., Montecino-Rodriguez, E., Kudryashova, E., Kramerova, I., Hoffman, E. P., Liu, S. D., Miceli, M. C., and Spencer, M. J. (2009). Osteopontin promotes fibrosis in dystrophic mouse muscle by modulating immune cell subsets and intramuscular TGF-beta. J Clin Invest 119, 1583-1594.
151. Vuga, L. J., Ben-Yehudah, A., Kovkarova-Naumovski, E., Oriss, T., Gibson, K. F., Feghali-Bostwick, C., and Kaminski, N. (2009). WNTSA is a regulator of fibroblast proliferation and resistance to apoptosis. Am J Respir Cell Mol Biol 41, 583-589.
152. Wynes, M. W., Edelman, B. L., Kostyk, A. G., Edwards, M. G., Coldren, C., Groshong, S. D., Cosgrove, G. P., Redente, E. F., Bamberg, A., Brown, K. K., et al. (2011). Increased cell surface Fas expression is necessary and sufficient to sensitize lung fibroblasts to Fas ligation-induced apoptosis: implications for fibroblast accumulation in idiopathic pulmonary fibrosis. J Immunol 187, 527-537.
153. Wynn, T. A., and Ramalingam, T. R. (2012). Mechanisms of fibrosis: therapeutic translation for fibrotic disease. Nat Med 18, 1028-1040.
154. Yamashita, C. M., Dolgonos, L., Zemans, R. L., Young, S. K., Robertson, J., Briones, N., Suzuki, T., Campbell, M. N., Gauldie, J., Radisky, D. C., et al. (2011). Matrix metalloproteinase 3 is a mediator of pulmonary fibrosis. Am J Pathol 179, 1733-1745.
155. Yang, J. J., Tao, H., Huang, C., and Li, J. (2013). Nuclear erythroid 2-related factor 2: a novel potential therapeutic target for liver fibrosis. Food Chem Toxicol 59, 421-427.
156. Yoshida, T., Ogata, H., Kamio, M., Joo, A., Shiraishi, H., Tokunaga, Y., Sata, M., Nagai, H., and Yoshimura, A. (2004). SOCS1 is a suppressor of liver fibrosis and hepatitis-induced carcinogenesis. J Exp Med 199, 1701-1707.
157. Yukawa, K., Kishino, M., Goda, M., Liang, X. M., Kimura, A., Tanaka, T., Bai, T., Owada-Makabe, K., Tsubota, Y., Ueyama, T., et al. (2005). STAT6 deficiency inhibits tubulointerstitial fibrosis in obstructive nephropathy. Int J Mol Med 15, 225-230.
158. Zardi, E. M., Navarini, L., Sambataro, G., Piccinni, P., Sambataro, F. M., Spina, C., and Dobrina, A. (2013). Hepatic PPARs: their role in liver physiology, fibrosis and treatment. Curr Med Chem 20, 3370-3396.
159. Zhang, G., Kernan, K. A., Collins, S. J., Cai, X., Lopez-Guisa, J. M., Degen, J. L., Shvil, Y., and Eddy, A. A. (2007). Plasmin(ogen) promotes renal interstitial fibrosis by promoting epithelial-to-mesenchymal transition: role of plasmin-activated signals. J Am Soc Nephrol 18, 846-859.
160. Zhang, Y. M., Bo, J., Taffet, G. E., Chang, J., Shi, J., Reddy, A. K., Michael, L. H., Schneider, M. D., Entman, M. L., Schwartz, R. J., et al. (2006). Targeted deletion of ROCK1 protects the heart against pressure overload by inhibiting reactive fibrosis. FASEB J 20, 916-925.
161. Zhou, H., Li, Y. J., Wang, M., Zhang, L. H., Guo, B. Y., Zhao, Z. S., Meng, F. L., Deng, Y. G., and Wang, R. Y. (2011). Involvement of RhoA/ROCK in myocardial fibrosis in a rat model of type 2 diabetes. Acta Pharmacol Sin 32, 999-1008.
162. Zhou, X., Hovell, C. J., Pawley, S., Hutchings, M. I., Arthur, M. J., Iredale, J. P., and Benyon, R. C. (2004). Expression of matrix metalloproteinase-2 and -14 persists during early resolution of experimental liver fibrosis and might contribute to fibrolysis. Liver Int 24, 492-501.

### Example 6: A YAP/TAZ-miR-130/301 Molecular Circuit Exerts Systems-Level Control of Fibrosis in a Network of Human Diseases

Increases in tissue stiffness and intracellular tension are controlled by molecular changes in the extracellular matrix (ECM) and are common features of fibrosis, as found in health and disease^{1, 2}. ECM remodeling is a complex process, occurring through changes in the balance between matrix deposition and matrix degradation and through collagen crosslinking enzymes such as lysyl oxidase (LOX). Two related transcriptional coactivators, YAP (Yes Associated Protein 1) and TAZ (or WWRT1) are crucial for mechanotransduction, a process that converts extracellular mechanical cues into intracellular signaling³ and regulates cellular proliferation and survival^{4, 5} as well as organ growth⁶. These coactivators have been increasingly appreciated as active factors that control ECM plasticity⁷ and even pathologic fibrosis⁸. Recently, we described a critical role of the control of extracellular matrix (ECM) stiffening by the mechanosensitive microRNA-130/301 family, as activated by YAP/TAZ, in promoting pulmonary hypertension (PH). Identification of such a self-amplifying feedback loop in PH leading to perivascular fibrosis suggested that similar molecular mechanisms involving extracellular biomechanical cues and microRNA (miRNA) activity play important roles in other fibrotic diseases.

More specifically, individual miRNA families often control multiple target genes and phenotypes, making them attractive candidates as upstream “master” regulators of seemingly diverse processes, including cell-cell and cell-matrix interactions⁹. Crosstalk between miRNA biology and the biomechanical ECM properties across tissue types, however, has been largely unexplored. Recently, we have utilized network theory to predict in silico those dominant miRNAs governing a specific disease gene network^{10, 11}. Given the broad scope of diseases where ECM plasticity may figure prominently, we hypothesized that remodeling of the extracellular matrix (ECM) could be a pathogenic feature shared among seemingly disparate disease conditions, and miRNAs could be an upstream regulator of this molecular cascade. In order to address these questions we developed an advanced network-based approach to search for a global miRNA regulator(s) of human fibrotic pathophenotypes across 137 different human diseases.

In doing so, we have now identified miR-130/301 as a master regulator of ECM biology across a cohort of diseases—all related by a shared signature of fibrosis-relevant genes. Overall, these results define this miRNA family as a crucial point of communication between biomechanical stress and fibrosis in a network of human diseases and thus emphasize the increasingly attractive candidacy of miR-130/301 for therapeutic targeting. Furthermore, our study provides novel experimental support for the notion that complex in vivo relationships deeply embedded in disease networks can be deciphered accurately from available molecular disease maps in silico, despite the noisy and often incomplete nature of such contemporary data.

### Results

Network Analysis Reveals that miR-130/301 Members Target a Shared Cohort of Fibrotic Genes Across Human Diseases Related to PH:

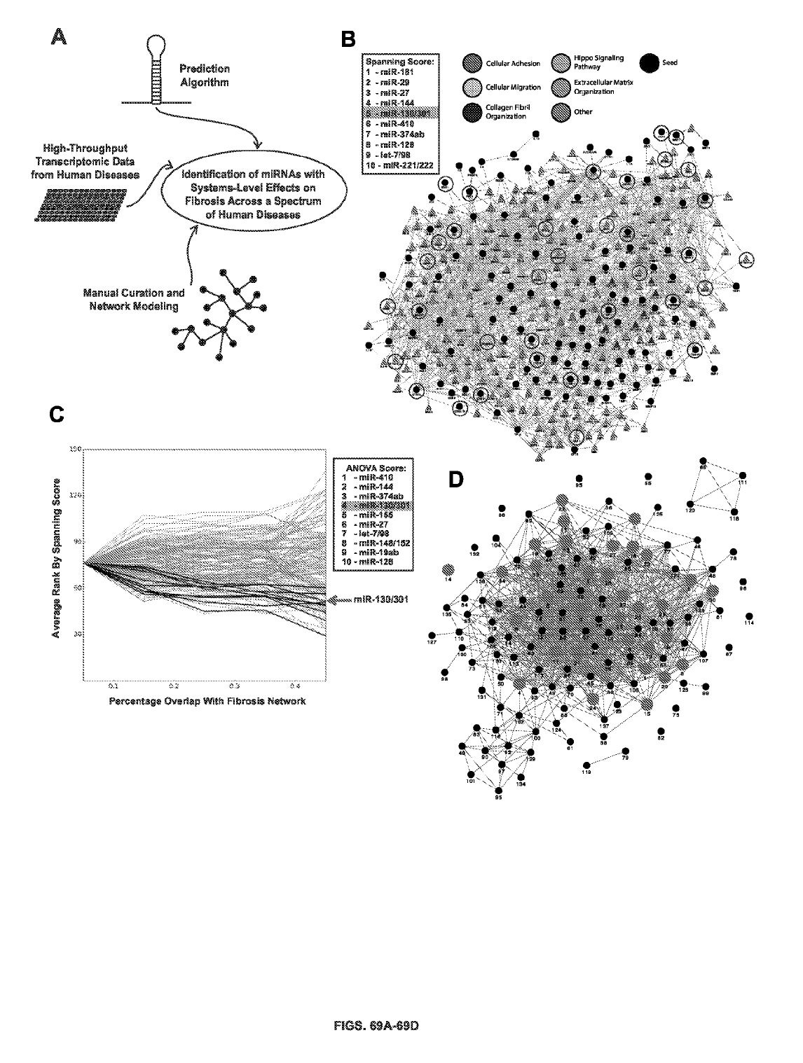

Given the importance of the YAP/TAZ-miR-130/301 circuit in PH (Bertero et al., submitted manuscript), we postulated that this feedback loop may be similarly active in controlling ECM plasticity in other fibrotic diseases. To define miRNAs that carry overarching regulatory control of tissue fibrosis across different diseases, we employed an in silico combination of miRNA target prediction, analysis of transcriptomic changes in 137 human diseases, and advanced gene network modeling (FIG. 69A). First, to identify miRNAs with functions that best span the entire extent of gene network, we previously described a “miRNA spanning score” (see Methods) based on the number and architectural distribution of predicted miRNA targets within that network¹¹. Utilizing an in silico “fibrosis network” (Bertero et al., submitted manuscript) constructed originally with curated seed genes known to be causatively involved in ECM remodeling and their first degree interactors (FIG. 69B), a broader component of factors related to ECM remodeling was evident in the predicted pool of miR-130/301 target genes and their related network neighbors.

To determine the relationship of these ECM-specific miRNA actions to other diseases, we compiled a suite of transcriptomic datasets of human diseased tissue as compared with normal controls (n=137) from publically available databases, representing a wide cross-section of human pathology (see Methods and Table 14). To control for statistical noise from wide variations in network size, specific disease gene networks were constructed by selecting the top 250 (by fold-change) significantly differentially expressed genes in each array compared with non-diseased controls. We cross-referenced these seed genes with the consolidated interactome and incorporated well-connected first-degree interactors, in order to “amplify” the molecular signal of these putative systems-level changes. Each disease network was categorized into one of four cohorts, as defined by increasing overlap with the fibrosis network. For each of the 137 disease-specific networks, miRNAs were then ranked by spanning score, as described above. With these data, we identified miR-130/301 family members among the most highly ranked miRNAs (Rank #4) with a robust one-way inverse correlation (one way ANOVA) between their assigned spanning score rank and the size of the fibrotic component for each disease network (FIG. 69C, Table 15). Coupled with the top ranking of this miRNA family by spanning score in relation to the fibrosis network directly (FIG. 69B), these findings predicted that miR-130/301 members are integral to the fibrotic program across a variety of diseases and tissue beds. Moreover, consistent with the known relevance of miR-130/301 in PH¹¹, among the 137 disease networks described above, a subset, ranked highly by their interconnectedness with the fibrosis network and the miR-130/301 family (Tables 15 and 16), was found to share a distinct cohort of fibrotic genes embedded in the overlap with a previously reported¹¹ PH disease gene network (FIG. 69D, Table 14). Thus, a combination of network analyses predicted a unique position for the miR-130/301 family at the intersection of fibrotic gene programming and this set of associated diseases.

miR-130/301 Expression Correlates with Activation of YAP/TAZ, the PPARγ-APOE-LRP8 Axis, and Matrix Stiffening in Pulmonary Fibrosis and Liver Fibrosis:

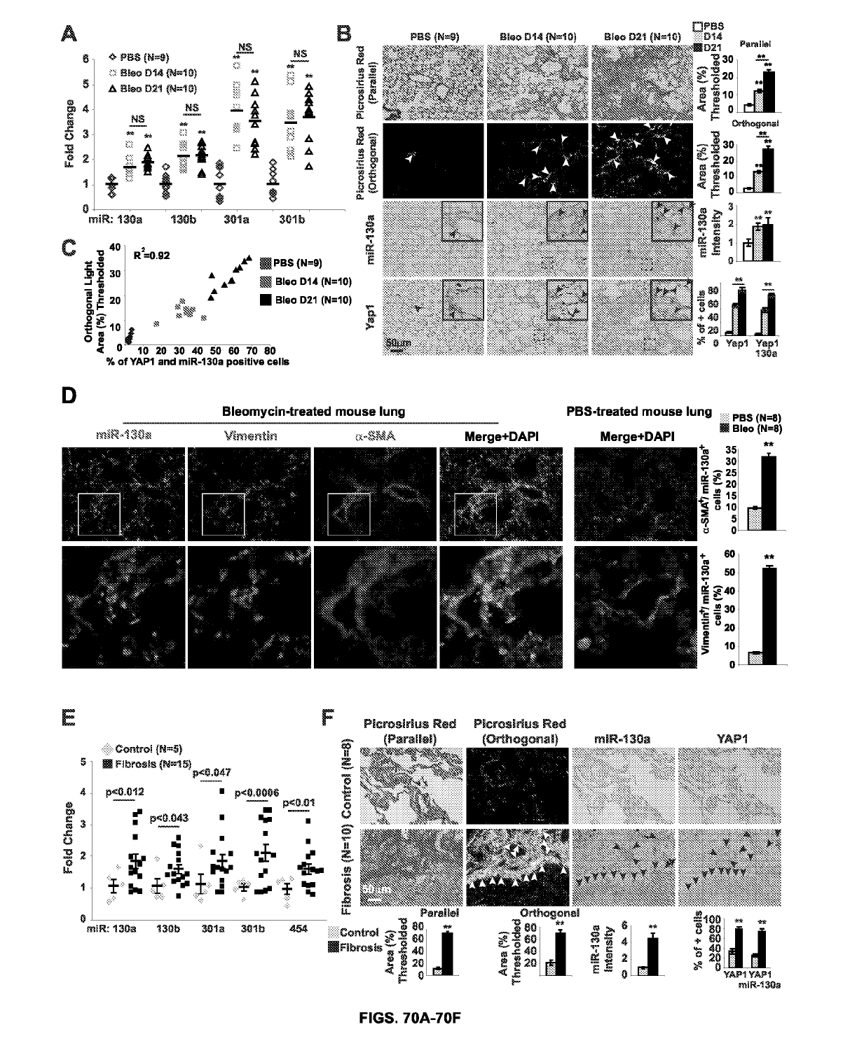

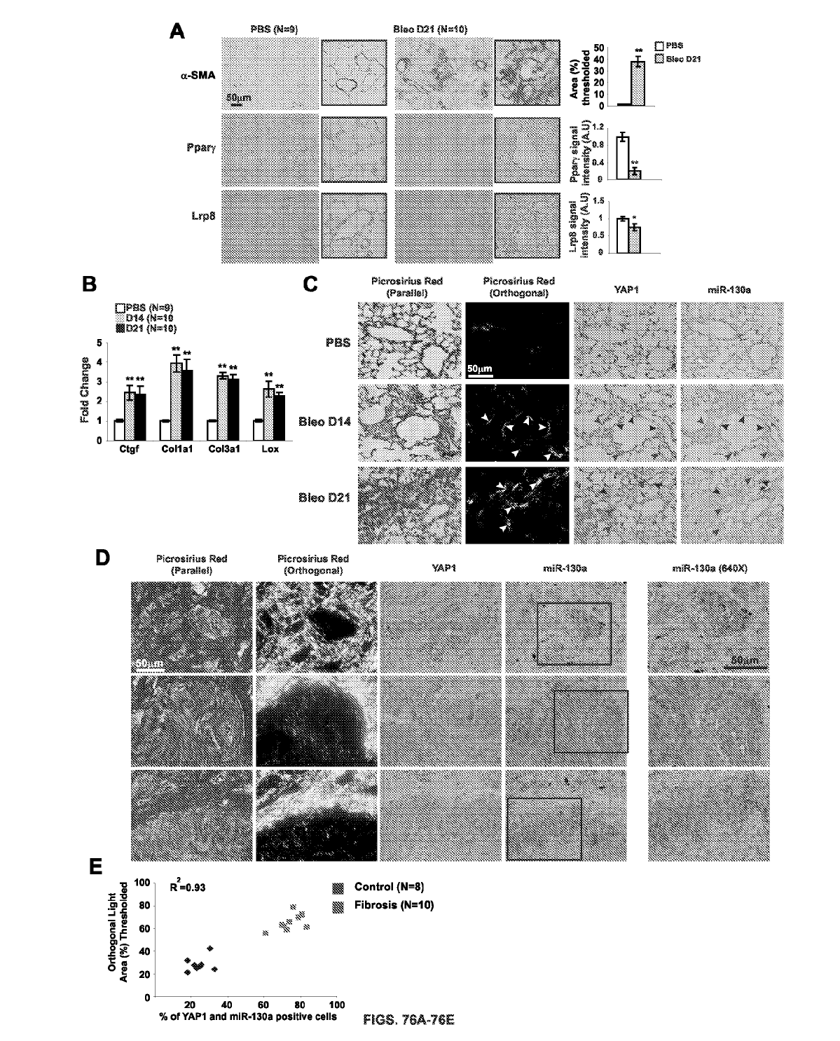

To demonstrate the putative unifying biology of miR-130/301 in this cohort of diseases, pulmonary fibrosis (Index Diseases #11 and #19, Table 14) and fibrotic liver disease (Index Disease #4, Table 14) were selected for further interrogation. In mouse models of bleomycin-induced pulmonary fibrosis (FIGS. 70A-70F), miR-130/301 expression was increased (FIGS. 70A-B). Consistent with induction of miR-130/301 by the mechanosensitive YAP/TAZ transcription factors in PH (Bertero et al., submitted manuscript), a positive correlation was observed among miR-130a expression, YAP nuclear localization, and downstream collagen crosslinking in pulmonary fibrosis (FIG. 70C). Furthermore, consistent with the direct down-regulation of the associated factors peroxisome proliferator-activated receptor gamma (PPAR), apolipoprotein E (APOE) and the apolipoprotein E receptor LRP8 by miR-130/301 in PH (Bertero et al., submitted manuscript), both Pparγ and Lrp8 were decreased in pulmonary fibrosis (FIG. 76). To define the exact cell types in which miR-130/301 was up-regulated, an in situ protocol was developed to stain simultaneously for miRNA and protein (see Methods). In the lung of mice treated with bleomycin, miR-130a expression was up-regulated in parenchymal fibroblasts, as demonstrated by co-localization of miR-130a with vimentin, a fibroblast marker, and α-smooth muscle actin (α-SMA), a marker of both activated fibroblasts and smooth muscle cells (FIG. 70D). Correspondingly, as predicted by our network algorithm (Index Diseases #11 and #19, Table 14), the same relationships between miR-130/301, YAP/TAZ, and collagen crosslinking were observed in pulmonary tissue derived from a cohort of patients suffering from idiopathic pulmonary fibrosis (FIG. 70E-F and FIGS. 76A-76E). Thus, beyond PH, the YAP/TAZ-miR-130/301 feedback loop is active in pulmonary fibrosis and specifically in fibroblasts anatomically far removed from the pulmonary vasculature itself.

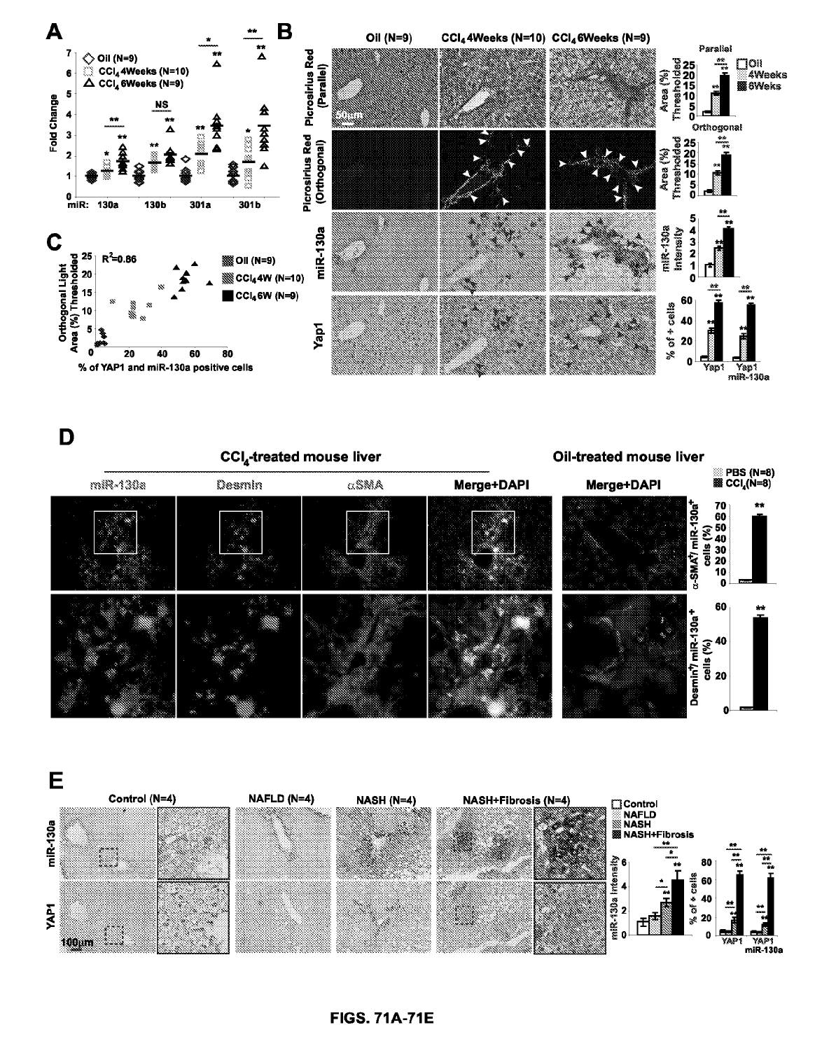

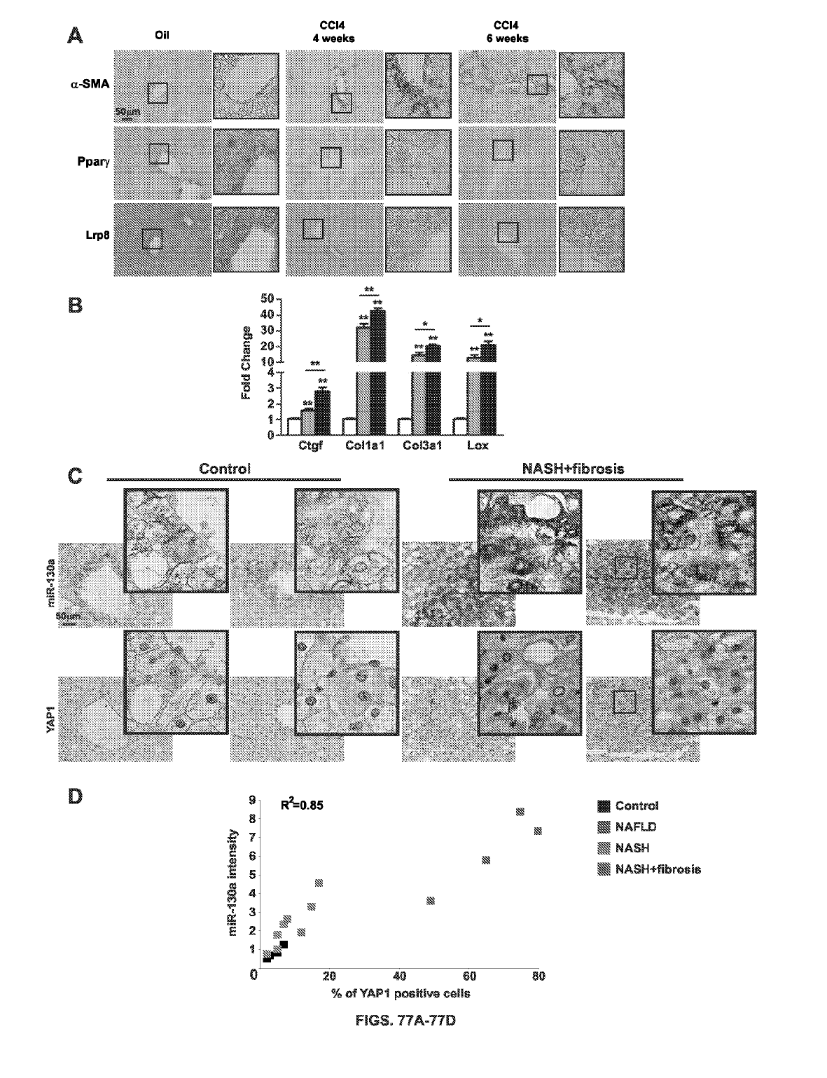

Similarly, in a mouse model of carbon tetrachloride (CCl₄)-induced liver fibrosis (FIGS. 71A-71E), miR-130/301 was increased (FIG. 71A-B) and Pparγ and Lrp8 were correspondingly decreased FIGS. 77A-77D), accompanied by a positive correlation among collagen crosslinking, miR-130a expression, and YAP nuclear localization (FIG. 71C). Via in situ liver stain of mice treated with CCl₄, miR-130a expression co-localized with both desmin, a stellate cell marker, and α-SMA (FIG. 71D). Similarly, as guided by our network predictions regarding various fibrotic liver diseases (for instance, Index Disease #4, Table 14), miR-130a and YAP were found to be increased in fibrotic human liver tissue—in this case stemming from nonalcoholic steatohepatitis (FIG. 71E). Thus, as delineated by our in silico predictions, distinct from PH, the YAP/TAZ-miR-130/301 circuit is activated across both pulmonary and hepatic diseases in animals and humans.

Forced miR-130/301 Expression Activates ECM Remodeling and Liver Fibrosis in Mice:

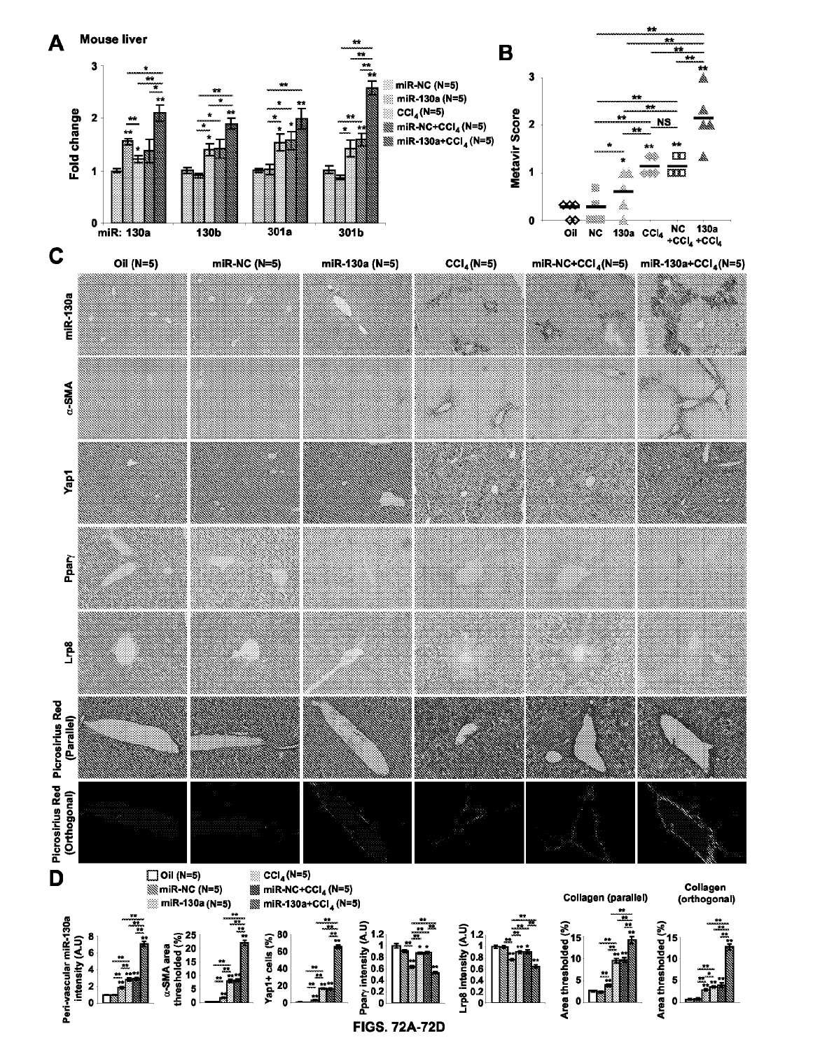

We previously demonstrated that forced expression of miR-130/301 in the lung of mice is sufficient to induce pulmonary hypertension¹¹ and pulmonary vessel fibrosis (Bertero et al., submitted manuscript). To determine if miR-130a is sufficient to induce liver fibrosis, chronic liver expression of miR-130a was studied. MiRNA delivery was achieved by serial (every 3 days during 4 weeks) intraperitoneal injections of liposomally encapsulated miR-130a oligonucleotide mimics in presence or absence of suboptimal dose of CCl₄ (0.1 mL per kg body by week). This protocol led to up-regulated miR-130a expression (but not other family members) in whole liver tissue (FIG. 72A). Such delivery down-regulated target gene expression of Pparγ and Lrp8 as well as modestly activated Yap1 nuclear localization and collagen crosslinking (FIG. 72B-D). These effects were enhanced by delivery of both miR-130a along with a decreased dose of CCl₄, promoting a robust down-regulation of Lrp8 and Pparγ and more substantially increased Yap1 activation and collagen crosslinking, compared to CCl₄ alone, CCl₄+miR-control (miR-NC), or miR-130a alone (FIG. 72C-D). Moreover, forced miR-130a and increased ECM stiffening was sufficient to activate a self-amplifying feedback loop and further increase endogenous miR-130/301 family expression (FIG. 72A). Taken together, consistent with the miR-130a-dependent actions in the pulmonary vascular space, these results demonstrated that this miRNA is sufficient to induce liver fibrosis as well.

Inhibition of the miR-130/301 Family Prevents ECM Remodeling and Disease Progression in Mouse Models of Pulmonary Fibrosis and Liver Fibrosis:

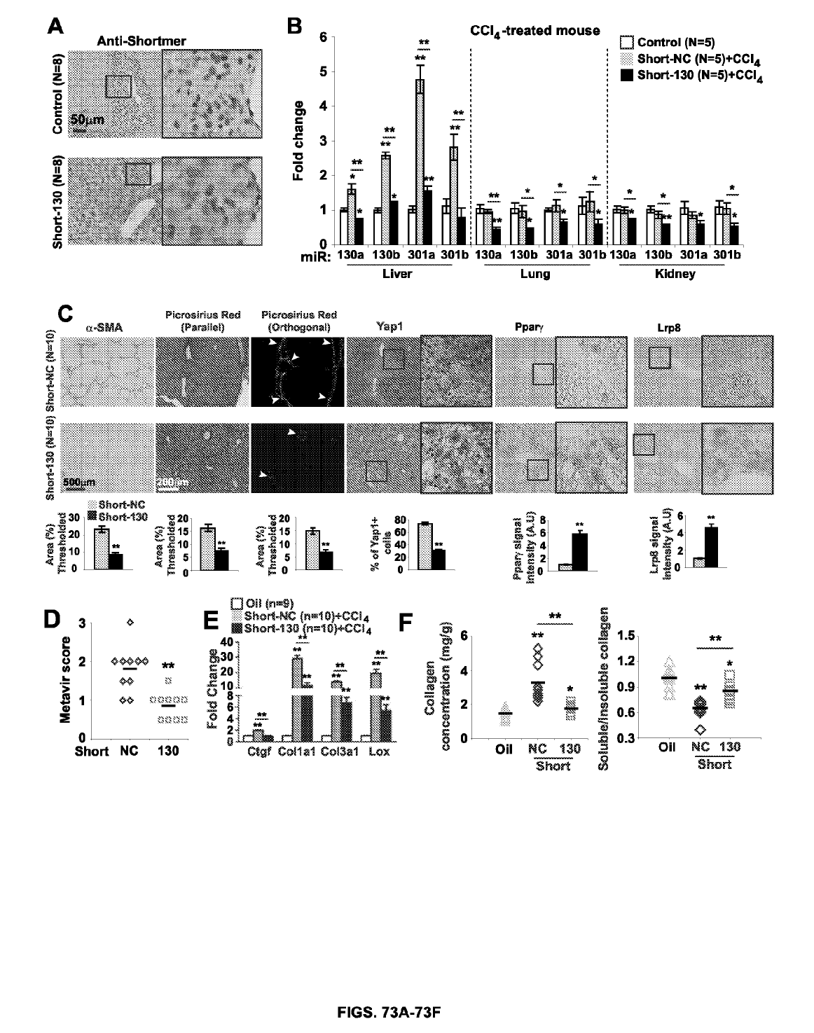

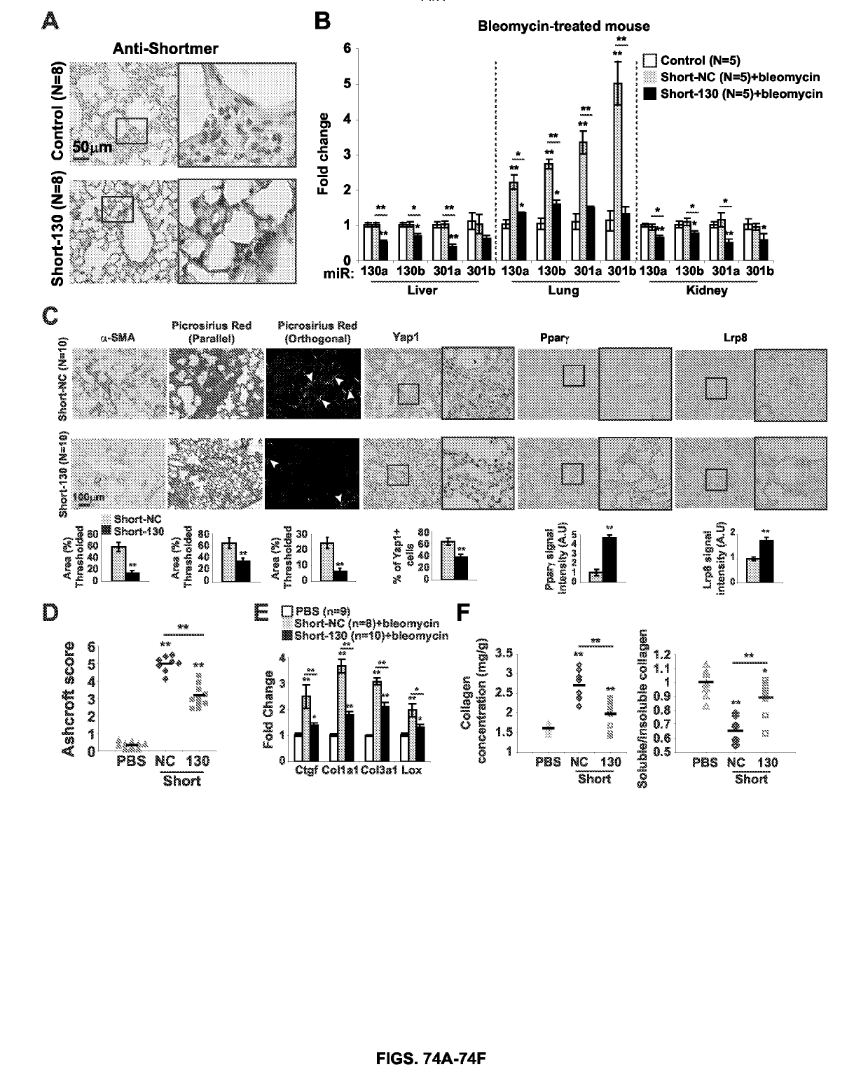

To determine whether miR-130/301 family members are necessary for control of fibrosis across both pulmonary and liver fibrosis, mice were serially administered control versus Short-130, an antisense oligonucleotide designed to inhibit all miR-130/301 family members in vivo in mouse liver (FIG. 73A-B) and mouse lung (FIG. 74A-B). In both models of pulmonary (bleomycin exposure) and liver (CCl₄ exposure) fibrosis, Short-130 inhibited miR-130/301 and reversed the down-regulation of miR-130/301 target genes Pparγ and Lrp8 (FIGS. 73A-73F and FIG. 74C), decreased Lox expression, reduced collagen expression and collagen crosslinking (FIGS. 73C, 73E, 73F, 74C, 74E and 74F), and reduced Yap nuclear localization (FIGS. 73C and 74C) and YAP activation, as reflected by CTGF expression (FIGS. 73E and 74E). In doing so, miR-130/301 inhibition significantly reduced end-stage fibrosis, as assessed by Metavir (FIG. 73D) score and Ashcroft score (FIG. 74D).

Pharmacologic Activation of APOE with LXR Agonist GW3965 Decreases Peri-Arteriolar Fibrosis and Improves Lung Fibrosis In Vivo.

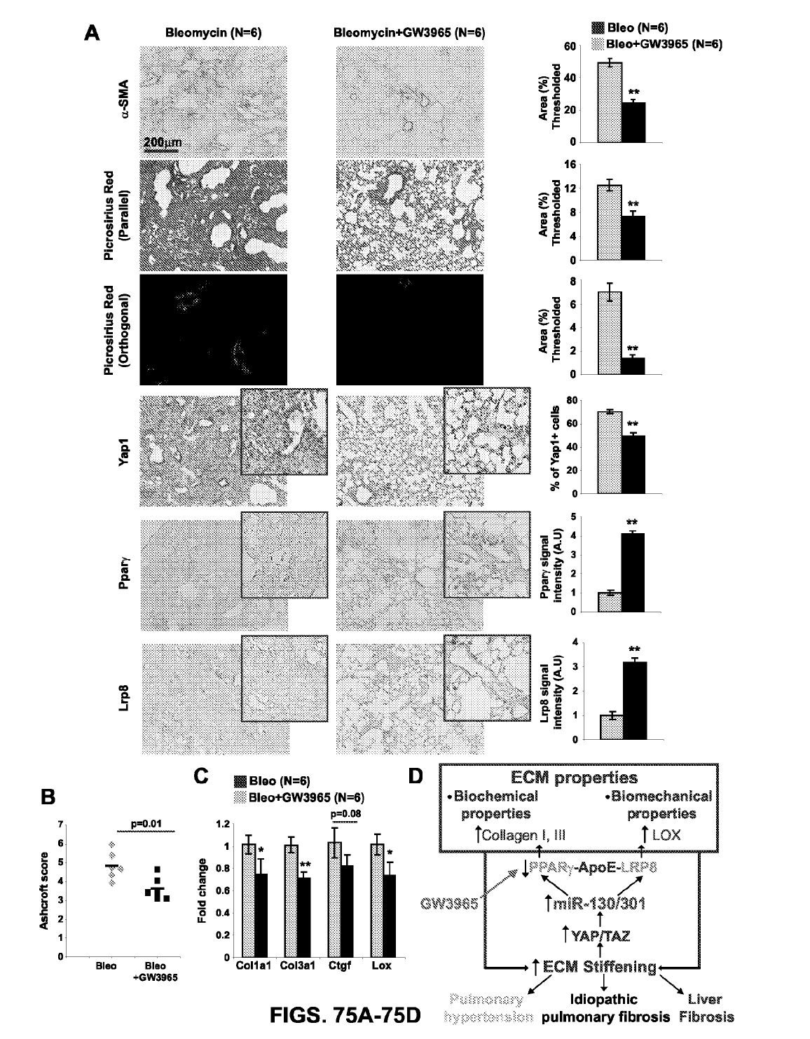

To determine whether downstream ApoE is critical for miR-130/301-induced fibrosis, we attempted to prevent lung fibrosis in bleomycin-exposed mice via treatment with a pharmacological activator of ApoE¹², the liver-X nuclear hormone receptor agonist GW3965 (FIG. 75). When GW3965 was administered serially after bleomycin exposure, collagen crosslinking (FIG. 75A) was inhibited, leading to decreased indices of lung fibrosis, as reflected by Ashcroft score (FIG. 75B). GW3965 treatment also reversed Pparγ and Lrp8 down-regulation (FIG. 75A) as well as reduced YAP nuclear localization (FIG. 75A) and YAP activation, as reflected by decreased Ctgf expression (FIG. 75C). Consequently, we can conclude that the YAP-TAZ-miR-130/301 circuit acts as a master regulator of both fibrotic gene programming and ECM remodeling across multiple pathobiological contexts in vivo. Moreover, by offering proof of our in silico predictions, these findings identify the fibrotic actions of the miR-130/301 family as a unifying molecular basis for the convergent relationship of seemingly disparate diseases (FIG. 75D).

### Discussion

By using network-based computational modeling and in vivo experimentation, we have defined the YAP/TAZ-miR-130/301 molecular circuit and its downstream control of ECM remodeling as a shared and unifying in vivo origin of a network of human diseases (FIG. 75D). Such identification of “network-based” regulators carries broad implications on the fundamental significance of ECM plasticity in the shared fibrotic origins linking seemingly disparate diseases. It also suggests the utility of related pharmacologic strategies (i.e., inhibitors of miR-130/301) for these associated diseases. Finally, the success of this approach provides much-anticipated experimental evidence for the clinical utility of re-conceptualizing diseases based on systems of shared gene networks and their molecular regulators¹³.

Our results showcase the power of advanced analysis of gene network architecture not only to predict a relevant fibrotic gene “program” shared among related human diseases but also to identify its overarching regulators, such as the miR-130/301 family, across those diseases (FIGS. 69A-69D and 75D). Currently, experimental evidence and expertise are only just emerging in support of using network theory to guide analysis across human diseases. There is increasing appreciation that “intermediate” phenotypes such as tissue fibrosis are common among human diseases previously thought to develop independently. The molecular overlap of complex human disease states has begun to be interrogated at a systems-wide level^{14, 15} but our ability to discern the existence of overarching “network-based” regulators across pathologic conditions has remained limited. In that vein, our network analysis also uncovered shared miR-130/301-specific commonalities among diseases that have rarely, if ever, been clinically associated with fibrosis (i.e., Ebola infection, schizophrenia, among others; Table 14). The putative connection of miR-130/301 and ECM biology to schizophrenia is particularly intriguing, as it is a disorder where ECM remodeling in perineuronal nets is already suspected to control final psychiatric manifestations¹⁶. Furthermore, it is possible that an even more complex and wide-reaching interactome exists among miR-130/301 and additional miRNAs predicted to recognize large portions of the same fibrotic disease network (FIGS. 69A-69D and Table 15). More detailed analyses of the architecture of that interactome could further reveal as-of-yet undescribed mechanistic connections between this miRNA family, ECM biology, and seemingly unrelated biologic processes. Future experimental validation in vivo of these principles would advance the concept of re-classifying diseases based on increasingly available data of molecular signatures that drive the origin of disease¹³.

The coupling of such network-based modeling with experimental validation also facilitated the identification of the YAP/TAZ-miR-130/301 circuit as a broad mediator of mechanotransduction across a variety of tissue beds and disease contexts. Isolated factors such as PPARγ¹⁷⁻¹⁹ and ApoE²⁰⁻²² as well as their downstream effectors CTGF²³ and the lysyl oxidase family²⁴⁻²⁶ previously have been implicated in diverse forms of pathologic fibrosis. However, their molecular interconnections with upstream regulators have been difficult to define. Separately, a growing appreciation has emerged regarding the activity of YAP/TAZ and microRNAs in general in regulating ECM biology. For instance, YAP/TAZ can drive fibrogenesis and tumorigenesis in an array of transformed tissue²⁷⁻²⁹ such as the liver³⁰ and non-transformed tissue including the lung⁸. Additionally, a cohort of so-called “fibromiRs” have been defined in specific cancer-related and non-cancer-related fibrotic diseases, including miR-155³¹, let-7³², miR-29³³⁻³⁵, miR-21³⁶⁻³⁸ and miR-199^{39, 40}, among others. Yet, only unique miRNAs, such as miR-29⁴¹ and miR-18⁴² have been found sensitive to YAP activity and tissue mechanics^{42, 43} in specific contexts such as breast cancer. Unlike prior studies of miR-130/301 family members focusing on specialized conditions such as liver cancer^{44, 45}, breast cancer^{46, 47}, scleroderma⁴⁸, our findings here emphasize the global dysregulation of the miR-130/301 family and its elusive interconnections with the molecular fibrotic machinery of the fibroblast. Given its adjustable, feedback-driven property, the miR-130/301-YAP/TAZ circuit may be responsible, at least in part, for individualized “tuning” of ECM remodeling observed among different fibrotic disorders. Given our growing appreciation of YAP/TAZ activity resulting from physical stimuli such as shear stress⁴⁹, it is also possible that miR-130/301 and other YAP/TAZ-associated miRNAs may constitute a newly defined set of factors responding to a wide range of physical alterations (i.e., vascular hemodynamics) of the microenvironment in addition to stiffness alone. Identification of these interconnections sheds a new light on the complexity of fibrotic diseases and highlights the increasingly attractive candidacy of YAP/TAZ⁵⁰ and miR-130/301^{11, 51, 52} for therapeutic targeting.

In regard to the relationship of this disease network to PH specifically, identification of the centrality of the YAP/TAZ-miR-130/301 circuit also provides molecular insight into the heterogenous clinical associations of secondary diseases found with this enigmatic vascular condition⁵³. For instance, World Health Organization (WHO) Group I pulmonary arterial hypertension (PAH) is a severe form of PH where disparate secondary diseases are catalogued together based on histopathological parallels rather than molecular similarities (including liver disease and portopulmonary hypertension among others). Alternatively, WHO Group III PH encapsulates a variety of pulmonary diseases including idiopathic pulmonary fibrosis (IPF) where hypoxia has been described as an environmental trigger linking lung pathology to PH. Yet, for a PH-related disease such as idiopathic pulmonary fibrosis (IPF), other unifying yet still unidentified molecular factors and/or cellular pathophenotypes beyond hypoxia likely figure prominently. Our data implicating the remodeling of the extracellular matrix (ECM) among this network of diseases form the foundation of two non-mutually exclusive models of how diseases such as pulmonary fibrosis and fibrotic liver diseases may intersect with PH. First, these diseases may develop in a cell autonomous fashion, driven by separate injuries [such as hypoxia, inflammation, and specific genetic mutations linked to PH¹¹] that induce miR-130/301 in distinct tissue beds. Thus, it is tempting to speculate that genetic mutations linked to PH and miR-130/301 (Bertero et al., submitted manuscript) may also contribute to separate fibrotic lung and liver diseases. A second model implicates non-cell autonomous methods of activating this molecular feedback loop among distinct anatomic tissue beds. For instance, in WHO Group III PH associated with pulmonary fibrosis, our results indicate that, independent of hypoxia, parenchymal fibrosis may activate the YAP/TAZ-miR-130/301 circuit in adventitial fibroblasts and perhaps other related mesenchymal stem cells⁵⁴, thus accelerating vascular stiffness. Moreover, given increased miR-130/301 levels in plasma of PH patients^{11, 55}, pathogenic transfer and endocrine signaling of miR-130/301 between lung and liver is an intriguing possibility. Both models may be active, and the identification of central factors common to all diseases provides a much needed molecular landmark to decipher additional complexities of disease interconnection.

Finally, the identification of a fibrotic gene program associated with a single set of miRNAs and shared across multiple diseases carries broad clinical ramifications beyond disease re-classification. At the diagnostic level, molecular screening for this fibrotic signature could facilitate the ability to identify persons at risk for such a related “syndrome” of diseases. At the therapeutic level, the efficacy of targeting individual fibrotic enzymes, such as matrix metalloproteinases (MMPs), has been suboptimal, especially when tested at the clinical stage^{56, 57}. Thus, a targeted pharmacologic combination of inhibiting the miR-130/301 family via shortmers⁵⁸ in addition to manipulating downstream miR-130/301-dependent pathways such as LXR/APOE activity (FIGS. 75A-75D) offers a rational avenue for cooperative therapeutic targeting of fibrosis that has yet to be achieved to date. Moreover, future biological confirmation of the network-based actions of miR-130/301 family may guide an even more powerful systems pharmacology approach to targeting fibrosis which has not yet been pursued in great depth.

Together, the results herein define the control of a fibrotic gene program by the YAP/TAZ-miR-130/301 circuit, shared among a network of related diseases. These findings re-define our conception of the elusive molecular origins of these diseases, thus offering much-needed molecular diagnostic and therapeutic opportunities. Moreover, by combining advanced network analyses with experimental interrogation, our results provide a roadmap to study systems-level cooperativity pervasive among miRNAs and the human disease network, in general. Such future work has the potential to uncover additional hidden yet fundamental links present in seemingly unrelated pathology.

### Methods

Collection and Analysis of Expression Array Data:

In order to include transcriptomic data in our statistical assessment of miRNA activity in fibrotic disease, we compiled a set of 137 expression arrays analyzing human tissue in a variety of disease conditions (see also Table 14). Publically available arrays were found in the Gene Expression Omnibus (GEO) database, filtered to include only analyses performed on untreated, biopsied human tissue and drawn blood (as compared with matched, non-diseased control), with one array per tissue type per disease. Diseases were excluded for which the only available tissue samples were not directly relevant to the pathology in question, as in the case of pulmonary hypertension (PH), where only peripheral blood samples from PH patients had been analyzed, as opposed to more disease-relevant solid tissue from the lung. The final dataset represented 137 distinct tissue environments across N diseases, and provided a broad cross-section of human pathology. We selected those genes in each array that showed a fold-change >+2.0 and a p-value<0.05, and cross-referenced the resulting gene set with the consolidated interactome to form a unique disease network.

Only the top 250 genes (by fold-change) were selected in cases where the number of significantly differentially expressed genes exceeded this number. These networks were then expanded to include well-connected first-degree interactors, as described above. Such truncation was necessary to optimize our ability to discern biologically meaningful molecular overlap, due to the inherent noise and effect saturation in comparing excessively large gene sets. First, because expression array analyses are susceptible to noise, we decreased the number of false-positives present in our network by including only those genes that show the greatest amount of modulation in disease. Second, because metrics such as the spanning score are based largely on the overlap of miRNA target pools with the network in question, an excessively large gene network would present a situation in which the upper end of the score spectrum can become saturated, thus increasing the difficulty in differentiating the relative influence of top-scoring miRNAs from one another. While inevitably some information would be lost in such truncation (leading to higher false-negative rate), we postulated that focusing on genes with the largest expression changes would provide a reasonable representation of the key, systems-level alterations in each diseased tissue. Furthermore, the process of first thresholding and then expanding the network by adding in well-connected first-degree interactors provided an additional method (along with the standard fold-change and p-value cutoffs described above) for isolating and amplifying the molecular signal of these putative systems-level changes. Thus, overall, we generated a conservative representation of the available expression data, favoring accurate disease networks with few false-positives over networks that are fully comprehensive.

For each of the 137 disease networks, we determined (a) the fraction of the network that overlapped with the fibrosis signature, and (b) the ranked list of miRNAs (by spanning score) that influenced the network. In order to isolate miRNAs that are specifically relevant to fibrosis (and not simply influential across all disease networks by virtue of having a large and well-connected target pool), we grouped disease networks into four cohorts according to the size of their overlap with the fibrosis signature. We then ranked miRNAs based on a one-way ANOVA means comparison test for their assigned spanning score rank for the networks in each disease cohort. miRNAs that scored well by this metric had higher average spanning scores in strongly fibrotic disease groups, relative to their performance overall. Upon identifying the miR-130/301 family as a critical regulator of fibrosis across disease contexts, we ranked each of the 137 disease networks according to (a) its overlap with the fibrosis network and (b) the spanning score rank assigned to miR-130/301 in that disease context. This was quantified as the average of two values: (1) the fraction of network genes that were shared with the fibrosis network and (2) the fraction of the highest possible rank achieved by miR-130/301 [1−rank₁₃₀/rank_{MAX}]. This ranking identified disease networks that contained significant fibrotic components and were heavily influenced by this miRNA family.

In Situ Hybridization:

The protocol for in situ hybridization for miRNA detection was based on a prior report¹¹. Specifically, 5 μm tissues sections were probed using a 3′ fluorescein isothiocyanate (FITC) labeled miRCURY LNA hsa-miR-130a detection probe (Exiqon). The miRCURY LNA scramble-miR probe was used as negative control. Following re-hydration (Sigma) tissues were formaldehyde-fixed (4% formaldehyde, Sigma) before inactivation of endogenous enzymes by acetylation buffer [873 uL of triethanolamine (Sigma) and 375 uL acetic anhydride (Fisher) in 75 ml distilled water]. Probe annealing (25 nM LNA probe) was performed in hybridization buffer (Sigma, H7782) for 16 hours at RNA-Tm-22° C. (62° C.). Following serial washes with 2×SSC, 1×SSC, and 0.5×SSC (Sigma) at 62° C., immunolabeling was performed with an anti-FITC biotinconjugated antibody for overnight at 4° C. (1:400; Sigma-Aldrich). For detection, development was achieved by adding streptavidin-biotinylated alkaline phosphatase complex (Vector Labs) followed by Nitro blue tetrazolium chloride/5-Bromo-4-chloro-3-indolyl phosphate substrate solution (NBT/BCIP, Roche), and positive staining was evident by a blue color. MiR-130a expression was quantified in the vascular wall of 15-20 pulmonary arteries or for liver tissue in 10 random 20× fields per animal using ImageJ software (NIH).

For co-detection of proteins and miRNA, after probe hybridization and following serial washes with SCC immunolabeling was performed with anti-α-SMA (1/500; Sigma-Aldrich), anti-vimentin (1/250; Abcam) and/or anti-Desmin (1/1000; Abcam) antibody for overnight at 4° C. After 3 washes in TBS Tween 0.1% slides were incubated with donkey anti-mouse and donkey anti-rabbit Alexa-conjugated antibody (Alexa 568 and Alexa 647) for 1 hour at room temperature. After 3 washes in TBS/Tween 0.1%, slides were mounted with anti-fading medium with DAPI (Vectashields, Vector).

Animals:

All animal treatments and analyses were conducted in a controlled and non-blinded manner.

Lung Fibrosis Model:

C57BL/6 mice (7-8 weeks old) were exposed to 0.035 U of bleomycin via oropharyngeal route. In the control group, saline (PBS) was administered via the same route. Mice were euthanized 14 days or 21 days after bleomycin administration.

Liver Fibrosis Model:

For chronic CCl₄-induced liver fibrosis, mice were injected (intra-peritoneal) with 1 mL per kg body weight sterile CCl₄ in a 1:5 ratio in corn oil or corn oil alone (control) every 5 days for 4-6 weeks. Livers were harvested 72 h after the last injection.

Inhibition of miR-130/301 in a Mouse Model of Pulmonary Fibrosis:

Eight-week-old mice (C57Bl6) were injected with bleomycin (1.5 U/kg Sigma Aldrich) followed by 10 intraperitoneal injections (every 2 days) of control or miR-130/301 shortmer oligonucleotides, designed as antisense inhibitors recognizing the seed sequence of this miRNA family (20 mg/kg/dose; Regulus). Shortmer generation was previously described¹¹. Two days after the last injection, lung tissue was harvested for RNA extraction or paraffin embedding.

Inhibition of miR-130/301 in a Mouse Model of Liver Fibrosis:

Eight-week-old mice (C57Bl6) were injected with CCl₄ (1 mL per kg of body weight) every 5 days accompanied by intraperitoneal injections (every 2 days) of control or miR-130/301 shortmer oligonucleotides (20 mg/kg/dose; Regulus). Two days after the last injection, liver tissue was harvested for RNA extraction or paraffin embedding.

Treatment of Mice with Oral Ingestion of the Liver-X Nuclear Hormone Receptor (LXR) Agonist GW3965:

To determine the effects of the LXR agonist GW3965 (Sigma-Aldrich) and consequent APOE induction on lung fibrosis, as previously described¹², mice were exposed to bleomycin (as described above) and simultaneously assigned to control chow or chow supplemented with GW3965 (Research Diets, Inc.) at doses of 100 mg of drug per kilogram of mouse per day (based on average daily intake of 3.5 μm of chow). After two weeks, harvest of lung tissue was performed for RNA/protein extraction or paraffin embedding.

Study Approval:

All animal experiments were approved by the Harvard Center for Comparative Medicine. All experimental procedures involving the use of human tissue were approved by Institutional Review Boards at Partners Healthcare, Boston Children's Hospital, National Institutes of Health, as well as the New England Organ Bank. Ethical approval for this study conformed to the standards of the Declaration of Helsinki. For formalin-fixed paraffin embedded lung samples and flash frozen samples, human idiopathic pulmonary fibrosis (IPF) specimens were collected from discarded surgical samples following lung transplantation. Control lung samples were collected in cases where non-IPF donor lungs were declined for transplantation. No additional clinical data were available. For formalin-fixed paraffin embedded liver samples, informed consent was obtained for standard-of-care wedge liver biopsy samples, collected from patients (Massachusetts General Hospital) undergoing weight loss surgery for the purpose of evaluating for nonalcoholic steatohepatitis (NASH). For this study, specimens were analyzed from unused surgical specimens that had previously been evaluated by a pathologist blinded to clinical data and scored as previously described^{59, 60}.

Statistics:

The number of animals in each group was calculated to measure at least a 20% difference between the means of experimental and control groups with a power of 80% and standard deviation of 10%. The number of unique patient samples for this study was determined primarily by clinical availability. In situ expression/histologic analyses of both rodent and human tissue, and pulmonary vascular hemodynamics in mice and rats were performed in a blinded fashion. Numerical quantifications for physiologic experiments using rodents or human reagents represent mean±standard error of the mean (SEM). Paired samples were compared by a 2-tailed student's t test. A P-value less than 0.05 was considered significant. Correlation analyses were performed by Pearson correlation coefficient calculation.

Network Construction:

The initial fibrosis network was constructed as previously described (Bertero et al., submitted manuscript). Briefly, we manually curated a set of 133 genes known fibrotic genes focusing only on genes known to play a causative role in tissue fibrosis. Interactions between curated genes were annotated according to a master list of protein-protein, protein-DNA, kinase-substrate, and metabolic interactions, drawn from several consolidated databases, referred to here as the “consolidated interactome”⁶¹. In order to capture any additional fibrotic genes that may have been missed in our initial curation, we also incorporated a select number of non-curated genes (“fibrosis interactors”) demonstrated to interact with a significant number of genes in our curated set. The resulting fibrosis network contained 350 nodes and 1459 edges, with a largest connected component of 339 nodes.

Network Clustering:

Clustering was performed using the Louvain method for community detection⁶³, as implemented in the NetworkX package for Python 3.3. The final partition was selected so as to maximize modularity in the graph. miRNA Target Prediction

miRNA target prediction was performed using the TargetScan 6.2 (Conserved) algorithm⁶⁴. The TargetScan algorithm detects mRNA with conserved complementarity to the “seed” (nucleotides 2-7) of a given miRNA. Because of this, miRNA that share a seed are grouped together as a family and regarded as a single unit by the algorithm. For this reason, we do not distinguish between miRNA belonging to the same family in any of our statistical analyses.

miRNA Spanning Score:

In order to rank the influence of miRNA families on the fibrosis network (and other disease networks), we ranked miRNAs based on their “spanning score”. As previously described⁶², this metric scores a miRNA family on three criteria: (1) the number of network genes which it targets, (2) the number of network clusters in which its targets reside, and (3) the hypergeometric p-value for the overlap of its target pool with the network. Each of these criteria is scored relatively, as a fraction of the maximum value achieved by any miRNA in our dataset for the network under consideration. This method provides a holistic assessment of the influence of a miRNA family on a given network of genes, considering not only the size, but also the spread and statistical significance of its target pool within the network.

Messenger RNA and miRNA Extraction:

Cells were homogenized in 1 ml of QiaZol reagent (Qiagen). Total RNA content, including small RNAs, was extracted using the miRNeasy kit (Qiagen) according to the manufacturer's instructions. Total RNA concentration was determined using a ND-1000 micro-spectrophotometer (NanoDrop Technologies)

Quantitative RT-PCR of Mature miRNAs:

Mature miRNA expression was evaluated using TaqMan MicroRNA Assays (Life Technologies/Applied Biosystems) and the Applied Biosystems 7900HT Fast Real Time PCR device (Life Technologies/Applied Biosystems). Expression levels were normalized to RNU48 or snoR55 for human or mouse experiments, respectively, and calculated using the comparative Ct method (2^{-ΔΔCt}).

Quantitative RT-PCR of Messenger RNAs:

Messenger RNAs were reverse transcribed using the Multiscript RT kit (Life Technologies) to generate cDNA. cDNA was amplified via fluorescently labeled Taqman primer sets using an Applied Biosystems 7900HT Fast Real Time PCR device. Fold-change of RNA species was calculated using the formula (2^{-ΔΔCt}), normalized to actin expression.

Lung and Liver Tissue Harvest:

After physiological measurements by direct right ventricular puncture, the pulmonary vessels were gently flushed with 1 cc of saline to remove the majority of blood cells, prior to harvesting cardiopulmonary tissue. The heart was removed, followed by dissection and weighing of the right ventricle (RV) and of the left ventricle+septum (LV+S). Organs were then harvested for histological preparation or flash frozen in liquid N2 for subsequent homogenization and extraction of RNA and/or protein. To further process lung tissue specifically, prior to excision, lungs were flushed with PBS at constant low pressure (˜10 mmHg) via right ventricular cannulation, followed by tracheal inflation of the left lung with 10% neutral-buffered formalin (Sigma-Aldrich) at a pressure of ˜20 cm H₂O. After excision and 16 hours of fixation in 10% neutral-buffered formalin at 25° C., lung and liver tissues were paraffin-embedded via an ethanol-xylene dehydration series, before being sliced into 5 μm sections (Hypercenter XP System and Embedding Center, Shandon).

Immunohistochemistry of Lung and Liver:

Lung and liver sections (5 μm) were deparaffinized and high temperature antigen retrieval was performed, followed by blocking in TBS/BSA 5%, 10% goat serum and exposure to primary antibody and biotinylated secondary antibody (Vectastain ABC kit, Vector Labs). A primary antibody against YAP1 (#4912; 1/200) was obtained from Cell Signaling. Primary antibodies against, LRP8 (ab115196; 1/100), α-SMA (1/400) were purchased from Abcam. Primary antibodies against PPARγ (sc-7273; 1/50) was purchased from Santa Cruz. Primary antibody against anti-shortmer (E5746-B3A; 1/1000) was provided by Regulus Therapeutics, as previously described². In most cases, color development was achieved by adding streptavidin-biotinylated alkaline phosphatase complex (Vector Labs) followed by Vector Red alkaline phosphatase substrate solution (Vector Labs). Levamisole was added to block endogenous alkaline phosphatase activity (Vector Labs). Pictures were obtained using an Olympus Bx51 microscope. For liver tissue and pulmonary tissue, 10 random 20× fields per animal were analyzed. Intensity of staining was quantified using ImageJ software (NIH). Degree of pulmonary fibrosis was assessed in paraffin-embedded lung sections stained for α-SMA by Ashcroft score⁶⁵. Degree of liver fibrosis was assessed in paraffin-embedded liver sections stained for α-SMA by Metavir score⁶⁵. All measurements were performed blinded to condition.

Picrosirius Red Stain and Quantification:

Picrosirius Red stain was achieved through the use of 5 μm paraffin sections stained with 0.1% Picrosirius Red (Direct Red80, Sigma) and counterstained with Weigert's hematoxylin to reveal fibrillar collagen. The sections were then serially imaged using with an analyzer and polarizer oriented parallel and orthogonal to each other. Microscope conditions (lamp brightness, condenser opening, objective, zoom, exposure time, and gain parameters) were constant throughout the imaging of all samples. A minimal threshold was set on appropriate control sections for each experiment in which only the light passing through the orthogonally-oriented polarizers representing fibrous structures (i.e., excluding residual light from the black background) was included. The threshold was maintained for all images across all conditions within each experiment. The area of the transferred regions that was covered by the thresholded light was calculated and at least 10 sections per condition were averaged together (Image J software).

Measurement of Collagen Content in Lung and Liver Tissue:

This protocol was adapted from a previously published protocol⁶⁶. Mouse lung or liver tissue was weighed, minced, and incubated in 0.5 M acetic acid at 4° C. After overnight digestion, the acetic acid-soluble and insoluble fractions were isolated by centrifugation. The soluble fraction was stored at −80° C., while the insoluble fraction was digested by overnight incubation in 6M hydrochloric acid at 85° C. Concentrations of soluble and insoluble (gelatinous) collagen fractions were determined using a Sircol Soluble Collagen Assay Kit (Biocolor) with a colorimetric reaction (measured at 550 nm) and a provided collagen reference standard curve.

TABLE 14
Disease networks ranked by their interconnectedness with the fibrosis network and the
miR-130/301 family. Distinct disease networks (n = 137) were constructed based on
expression profiling of diseased human tissue. These data were generated from the
Gene Expression Omnibus (GEO) database. Diseases were ranked based on the
influence of the miR-130/301 family (by spanning score rank) and their percentage
overlap with the fibrosis network (quantified as the average of two values: (1)
the fraction of network genes that are fibrotic and (2) the fraction of the
highest possible rank achieved by miR-130/301 [1 − rank₁₃₀/rank_{MAX}]).

| | | | Percentage | Network | miR-130/301 | |
|---|---|---|---|---|---|---|
| Index | Disease | Tissue | Fibrotic | Size | Rank | GEO ID |
| 1 | Helicobacter Pylori | Corpus Gastric | 0.677 | 130 | 10 | GDS625 |
| | Infection | Biopsy | | | | |
| 2 | Cerebral Palsy | Gracilis | 0.414 | 500 | 4 | GDS4353 |
| | | Muscle | | | | |
| 3 | Zaire | Macrophage | 0.413 | 326 | 5 | GDS4356 |
| | Ebolavirus | | | | | |
| | Infection | | | | | |
| 4 | Alcoholic | Liver Tissue | 0.485 | 132 | 17 | GDS4389 |
| | Hepatitis | | | | | |
| 5 | Pterygium | Conjunctiva | 0.44 | 386 | 14 | GDS1758 |
| 6 | Pituitary | Tumor Tissue | 0.388 | 500 | 9 | GDS4275 |
| | Gonadotrope | | | | | |
| 7 | Pituitary Null | Tumor Tissue | 0.336 | 500 | 3 | GDS4275 |
| | Cell Tumor | | | | | |
| 8 | Helicobacter Pylori | Antumn | 0.388 | 258 | 11 | GDS625 |
| | Infection | Gastric Biopsy | | | | |
| 9 | Idiopathic | CD3 Cells | 0.328 | 500 | 3 | GDS390 |
| | Thrombocytopenic | | | | | |
| | Purpura, | | | | | |
| | Remission | | | | | |
| 10 | Tuberous | Fibroblasts | 0.36 | 500 | 8 | GDS3281 |
| | Sclerosis | | | | | |
| | Complex | | | | | |
| | Periungual | | | | | |
| | Fibroma | | | | | |
| 11 | Idiopathic | Whole Lung | 0.335 | 478 | 10 | GDS1252 |
| | Pulmonary | | | | | |
| | Fibrosis | | | | | |
| 12 | Acute Rotavirus | Peripheral | 0.281 | 420 | 2 | GDS2048 |
| | Infection | Blood | | | | |
| | | Mononuclear | | | | |
| | | Cells | | | | |
| 13 | Familial Combined | Peripheral | 0.328 | 378 | 12 | GDS946 |
| | Hyperlidemia | Blood | | | | |
| 14 | Autism Fragile | Lymphoblastoid | 0.26 | 500 | 3 | GDS2824 |
| | X Type | Cells | | | | |
| 15 | Schizophrenia | Postmortem | 0.357 | 140 | 18 | GDS3345 |
| | | Prefrontal | | | | |
| | | Cortex | | | | |
| 16 | Tuberous | Fibroblasts | 0.3 | 500 | 10 | GDS3281 |
| | Sclerosis | | | | | |
| | Complex | | | | | |
| | Angiofibroma | | | | | |
| 17 | Dermatomyositis | Skeletal | 0.328 | 500 | 15 | GDS2153 |
| | | Muscle Biopsy | | | | |
| 18 | ER Negative | Tumor Tissue | 0.232 | 500 | 1 | GDS3716 |
| | Breast Cancer | | | | | |
| 19 | Idiopathic | Fibroblasts | 0.311 | 446 | 15 | GDS1012 |
| | Pulmonary | | | | | |
| | Fibrosis | | | | | |
| 20 | Bipolar | Postmortem | 0.422 | 128 | 33 | GDS3345 |
| | Disorder | Prefrontal | | | | |
| | | Cortex | | | | |
| 21 | Head and Neck | Tumor Biopsy | 0.382 | 404 | 28 | GDS3838 |
| | Squamous | | | | | |
| | Carcinoma | | | | | |
| 22 | ER Positive | Tumor Tissue | 0.252 | 500 | 10 | GDS3716 |
| | Breast Cancer | | | | | |
| 23 | UV Exposure | Skin | 0.376 | 500 | 29 | GDS400 |
| | | Fibroblasts | | | | |
| 24 | Morbid Obesity | Omental | 0.244 | 500 | 9 | GDS3679 |
| | | Adipose Tissue | | | | |
| 25 | Idiopathic | CD3 Cells | 0.256 | 500 | 11 | GDS390 |
| | Thrombocytopenic | | | | | |
| | Purpura, | | | | | |
| | Active | | | | | |
| 26 | Uterine Fibroid | Uterine | 0.32 | 500 | 21 | GDS484 |
| | | Leiomyoma | | | | |
| | | Tissue | | | | |
| 27 | Air Pollution | Peripheral | 0.256 | 500 | 12 | GDS3325 |
| | Exposure, Child | Blood | | | | |
| 28 | Scleroderma- | Fibroblasts | 0.267 | 480 | 15 | GDS1012 |
| | Associated | | | | | |
| | Pulmonary | | | | | |
| | Fibrosis | | | | | |
| 29 | Protein | Skeletal | 0.197 | 426 | 5 | GDS2868 |
| | Deficiency | Muscle | | | | |
| 30 | Aging | Frontal Cortex | 0.26 | 500 | 16 | GDS707 |
| | | Male | | | | |
| 31 | Primary | B Cells | 0.192 | 500 | 6 | GDS2723 |
| | Immunodeficiency | | | | | |
| | Syndrome | | | | | |
| 32 | Gastric Cancer | Tumor Tissue | 0.36 | 500 | 32 | GDS1210 |
| 33 | Idiopathic | Spinal Cord | 0.38 | 500 | 36 | GDS412 |
| | Amyotrophic | Gray Matter | | | | |
| | Lateral | | | | | |
| | Sclerosis | | | | | |
| 34 | Smoking | Macrophage | 0.248 | 500 | 16 | GDS3496 |
| 35 | Non-Melanoma | Actinic | 0.208 | 500 | 10 | GDS2200 |
| | Skin Cancer | Keratotic | | | | |
| | | Lesion | | | | |
| 36 | Osteoarthritis | Chondrocytes, | 0.232 | 500 | 14 | GDS3758 |
| | | Monolayer | | | | |
| | | Culture | | | | |
| 37 | MELAS | Muscle Biopsy | 0.217 | 498 | 12 | GDS1065 |
| | Syndrome | | | | | |
| 38 | Melanoma | Biopsy | 0.196 | 500 | 9 | GDS1989 |
| | InSitu | | | | | |
| 39 | Aging | Frontal Cortex | 0.276 | 500 | 22 | GDS707 |
| | | Female | | | | |
| 40 | Typhus | HMEC1 | 0.228 | 500 | 15 | GDS3848 |
| | Infection | Endothelial | | | | |
| | | Cells | | | | |
| 41 | Juvenile | Synovial Fluid | 0.384 | 500 | 39 | GDS711 |
| | Polyarticular | | | | | |
| | Rheumatoid | | | | | |
| | Arthritis | | | | | |
| 42 | Cerebral Palsy | Semitendinosus | 0.328 | 500 | 31 | GDS4353 |
| | | Muscle | | | | |
| 43 | Papillary | Tumor Tissue | 0.236 | 500 | 17 | GDS1732 |
| | Thyroid Cancer | | | | | |
| 44 | Severe Acute | Peripheral | 0.316 | 500 | 30 | GDS1028 |
| | Respiratory | Blood | | | | |
| | Syndrome | Mononuclear | | | | |
| | | Cells | | | | |
| 45 | Rheumatoid | Synovial | 0.296 | 500 | 27 | GDS2126 |
| | Arthritis | Tissues | | | | |
| 46 | Progressive | Muscle Biopsy | 0.176 | 500 | 9 | GDS1065 |
| | External | | | | | |
| | Opthalmoplegia | | | | | |
| 47 | HIV-1 Infection | Macrophage | 0.293 | 198 | 28 | GDS3511 |
| 48 | Parkinsons | Inferior | 0.26 | 500 | 23 | GDS4154 |
| | Disease | Olivary | | | | |
| | | Nucleus | | | | |
| 49 | Mild Cystic | Nasal | 0.188 | 500 | 12 | GDS2143 |
| | Fibrosis | Respiratory | | | | |
| | | Epithelium | | | | |
| 50 | Breast Cancer | Stromal Tissue | 0.35 | 40 | 37 | GDS4114 |
| 51 | Chlamydia | Dendritic Cells | 0.236 | 500 | 24 | GDS3573 |
| | Pneumonia | | | | | |
| | Infection | | | | | |
| 52 | Essential | Platelet | 0.212 | 500 | 22 | GDS1376 |
| | Thrombocythemia | | | | | |
| 53 | Hepatitis C | Huh8 | 0.346 | 182 | 44 | GDS4160 |
| | Virus Infecton | Hepatoma | | | | |
| | | Cells | | | | |
| 54 | Diabetic | Glomeruli | 0.332 | 500 | 42 | GDS961 |
| | Nephropathy | | | | | |
| 55 | Teratozoospermia | Sperm Cells | 0.08 | 500 | 5 | GDS2697 |
| 56 | Wilms Tumor | Tumor Tissue | 0.264 | 500 | 34 | GDS1791 |
| 57 | Juvenile | Synovial Fluid | 0.4 | 500 | 55 | GDS711 |
| | Pauciarticular | | | | | |
| | Rheumatoid | | | | | |
| | Arthritis | | | | | |
| 58 | Scott Syndrome | B Lymphoblast | 0.366 | 454 | 50 | GDS1320 |
| 59 | Vulvar | Lesion Biopsy | 0.104 | 500 | 10 | GDS2418 |
| | Intraepithelial | | | | | |
| | Neoplasia | | | | | |
| 60 | Acute Myeloid | Bone Marrow | 0.187 | 182 | 23 | GDS3057 |
| | Leukemia | | | | | |
| 61 | Pediatric | Omental | 0.24 | 500 | 32 | GDS3688 |
| | Obesity | Adipose Tissue | | | | |
| 62 | Exposure To | Cultured | 0.2 | 500 | 26 | GDS3868 |
| | Laminar Shear | HUVECs | | | | |
| | Stress | | | | | |
| 63 | Juvenile | Skeletal | 0.084 | 500 | 9 | GDS3417 |
| | Dermatomyositis, | Muscle | | | | |
| | Long | | | | | |
| | Duration | | | | | |
| 64 | Amyotrophic | Muscle Biopsy | 0.06 | 500 | 6 | GDS2855 |
| | Lateral | | | | | |
| | Sclerosis | | | | | |
| 65 | Oligodendroglioma | Tumor Tissue | 0.02 | 500 | 1 | GDS1813 |
| 66 | Non-Melanoma | Squamous Cell | 0.316 | 500 | 47 | GDS2200 |
| | Skin Cancer | Carcinoma | | | | |
| | | Lesion | | | | |
| 67 | Myelodysplastic | Bone Marrow | 0.32 | 500 | 48 | GDS1392 |
| | Syndrome | CD34 | | | | |
| | | Progenitor | | | | |
| | | Cells | | | | |
| 68 | Juvenile | Skeletal | 0.068 | 500 | 10 | GDS3417 |
| | Dermatomyositis, | Muscle | | | | |
| | Short | | | | | |
| | Duration | | | | | |
| 69 | Juvenile | Synovial Fluid | 0.3 | 500 | 46 | GDS711 |
| | Spondylo- | | | | | |
| | arthropathy | | | | | |
| 70 | Endometrioma | Endometrial | 0.024 | 500 | 4 | GDS3975 |
| | | Tissue | | | | |
| 71 | Sepsis | Skeletal | 0.212 | 424 | 33 | GDS3463 |
| | | Muscle | | | | |
| 72 | Presymptomatic | Peripheral | 0.244 | 500 | 38 | GDS2362 |
| | Malaria | Blood | | | | |
| | | Mononuclear | | | | |
| | | Cells | | | | |
| 73 | Cigarette | Oral Mucosa | 0.08 | 50 | 13 | GDS3709 |
| | Smoking, | | | | | |
| | Female | | | | | |
| 74 | Acne | Inflammatory | 0.26 | 470 | 41 | GDS2478 |
| | | Papules | | | | |
| 75 | Non-Union | Callous Bone | 0.012 | 500 | 4 | GDS369 |
| | Skeletal | | | | | |
| | Fracture | | | | | |
| 76 | Epstein-Barr | Tumor Tissue | 0.26 | 500 | 44 | GDS3610 |
| | Positive | | | | | |
| | Nasopharangeal | | | | | |
| | Carcinoma | | | | | |
| 77 | Emphysema | Lung Tissue | 0.296 | 500 | 50 | GDS737 |
| 78 | Respiratory | Bronchial | 0.204 | 500 | 36 | GDS2023 |
| | Syncytial Virus | Epithelial Cell | | | | |
| | Infection | Culture | | | | |
| 79 | Ulcerative | Descending | 0.06 | 500 | 14 | GDS3268 |
| | Colitis | Colon, | | | | |
| | | Uninflamed | | | | |
| 80 | Idiopathic | Hematopoietic | 0.232 | 500 | 41 | GDS2397 |
| | Myelofibrosis | CD34 Stem | | | | |
| | | Cells | | | | |
| 81 | Multiple | Brain Lesion | 0.08 | 500 | 18 | GDS4218 |
| | Sclerosis | | | | | |
| 82 | Alzheimers | Hippocampal | 0.212 | 500 | 39 | GDS4136 |
| | | CA1 Gray | | | | |
| | | Matter | | | | |
| 83 | Spastic | Muscle Biopsy | 0.012 | 500 | 9 | GDS2855 |
| | Paraplegia | | | | | |
| 84 | Autosomal | Polymorphonuclear | 0.124 | 500 | 27 | GDS3820 |
| | Dominant | Cells | | | | |
| | Monocytopenia | | | | | |
| 85 | Familial | Spinal Cord | 0.22 | 500 | 42 | GDS412 |
| | Amyotrophic | Gray Matter | | | | |
| | Lateral | | | | | |
| | Sclerosis | | | | | |
| 86 | Type 2 Diabetes | Pancreatic | 0.076 | 500 | 20 | GDS4337 |
| | | Islets | | | | |
| 87 | Preeclampsia | Placental | 0.136 | 500 | 31 | GDS3467 |
| | | Tissue | | | | |
| 88 | Morbid Obesity | Subcutaneous | 0.297 | 128 | 57 | GDS3679 |
| | | Adipose Tissue | | | | |
| 89 | Carious Pulpal | Dental Pulp | 0.264 | 500 | 53 | GDS1850 |
| | Tissue | | | | | |
| 90 | Atopic | Lesional Skin | 0.012 | 500 | 15 | GDS2382 |
| | Dermatitis | | | | | |
| 91 | Acute Myeloid | Peripheral | 0.225 | 376 | 48 | GDS3057 |
| | Leukemia | Blood | | | | |
| 92 | Acute | Muscle Biopsy | 0.112 | 500 | 32 | GDS2855 |
| | Quadriplegic | | | | | |
| | Myopathy | | | | | |
| 93 | Vertical Growth | Lesion Biopsy | 0.04 | 500 | 22 | GDS1989 |
| | Phase | | | | | |
| | Melanoma | | | | | |
| 94 | Invasive Ductal | Tumor Biopsy | 0.092 | 500 | 30 | GDS3853 |
| | Carcinoma | | | | | |
| 95 | Aldosterone- | Tumor Biopsy | 0.032 | 500 | 22 | GDS2860 |
| | Producing | | | | | |
| | Adenoma | | | | | |
| 96 | Osteoarthritis | Chondrocytes, | 0.056 | 500 | 26 | GDS3758 |
| | | Matrix Culture | | | | |
| 97 | Becker | Muscle Biopsy | 0.112 | 500 | 35 | GDS2855 |
| | Muscular | | | | | |
| | Dystrophy | | | | | |
| 98 | Inflammatory | Endomyocardium | 0.012 | 500 | 20 | GDS2154 |
| | Dilated | | | | | |
| | Cardiomyopathy | | | | | |
| 99 | Obesity, Female | Cultured | 0.232 | 392 | 54 | GDS1497 |
| | | Abdominal | | | | |
| | | Adipocytes | | | | |
| 100 | Clear Cell Renal | Tumor Tissue | 0.012 | 500 | 21 | GDS2881 |
| | Cell Carcinoma | | | | | |
| | Stage II | | | | | |
| 101 | Severe Cystic | Nasal | 0.008 | 500 | 21 | GDS2143 |
| | Fibrosis | Respiratory | | | | |
| | | Epithelium | | | | |
| 102 | Clear Cell Renal | Tumor Tissue | 0.02 | 500 | 23 | GDS2881 |
| | Cell Carcinoma | | | | | |
| | Stage I | | | | | |
| 103 | Malignant | Pleura | 0.052 | 500 | 29 | GDS1220 |
| | Pleural | | | | | |
| | Mesothelioma | | | | | |
| 104 | Polycystic | Granulosa | 0.14 | 500 | 44 | GDS4399 |
| | Ovary | Cells | | | | |
| | Syndrome | | | | | |
| 105 | Abdominal | Abdominal | 0.172 | 500 | 49 | GDS2838 |
| | Aortic | Aorta | | | | |
| | Aneurysm | | | | | |
| 106 | Testicular | Tumor Biopsy | 0.144 | 500 | 45 | GDS2842 |
| | Seminoma | | | | | |
| 107 | Depression | Postmortem | 0.113 | 106 | 41 | GDS3345 |
| | | Prefrontal | | | | |
| | | Cortex | | | | |
| 108 | Parkinson's | Dorsal Motor | 0.176 | 500 | 51 | GDS4154 |
| | Disease | Nucleus of the | | | | |
| | | Vagus | | | | |
| 109 | Symptomatic | Peripheral | 0.18 | 500 | 52 | GDS2362 |
| | Malaria | Blood | | | | |
| | | Mononuclear | | | | |
| | | Cells | | | | |
| 110 | Influenza | Whole Blood | 0.212 | 500 | 57 | GDS3919 |
| | Infection | | | | | |
| 111 | Glioblastoma | Tumor Tissue | 0.008 | 500 | 28 | GDS1813 |
| 112 | Sjogrens's | Minor Salivary | 0.164 | 500 | 53 | GDS3940 |
| | Syndrome | Gland | | | | |
| 113 | Ductal | Tumor Biopsy | 0.036 | 500 | 34 | GDS3853 |
| | Carcinoma | | | | | |
| | InSitu | | | | | |
| 114 | Hereditary | Gingival | 0.164 | 500 | 55 | GDS1685 |
| | Gingival | Tissue | | | | |
| | Fibromatosis | | | | | |
| 115 | X-Linked | Basal Ganglia | 0.064 | 500 | 41 | GDS1912 |
| | Recessive | | | | | |
| | Dystonia- | | | | | |
| | Parkinsonism | | | | | |
| 116 | Hutchinson- | Fibroblasts | 0.068 | 500 | 43 | GDS1504 |
| | Gilford Progeria | | | | | |
| | Syndrome | | | | | |
| 117 | Psoriasis | Lesional Skin | 0.021 | 94 | 36 | GDS3539 |
| 118 | Astrocytic | Tumor Tissue | 0.024 | 500 | 41 | GDS1813 |
| | Tumor | | | | | |
| 119 | Ulcerative | Descending | 0.036 | 500 | 44 | GDS3268 |
| | Colitis | Colon, | | | | |
| | | Inflamed | | | | |
| 120 | Anaplastic | Tumor Tissue | 0.068 | 500 | 52 | GDS1813 |
| | Oligoastrocytoma | | | | | |
| 121 | Oral Squamous | Laser Capture | 0.184 | 500 | 70 | GDS1584 |
| | Cell Carcinoma | Cell Isolates | | | | |
| 122 | Lethal | Spinal Cord | 0.064 | 500 | 52 | GDS1295 |
| | Congenital | | | | | |
| | Contracture | | | | | |
| | Syndrome | | | | | |
| 123 | Air Pollution | Peripheral | 0.128 | 500 | 63 | GDS3325 |
| | Exposure, Adult | Blood | | | | |
| 124 | Sickle Cell | Platelets | 0.039 | 204 | 50 | GDS3318 |
| | Disease | | | | | |
| 125 | Limb | Skeletal | 0.072 | 500 | 56 | GDS2083 |
| | Immobilization | Muscle | | | | |
| 126 | Rett Syndrome | Frontal Cortex | 0.07 | 454 | 57 | GDS2613 |
| 127 | Influenza | Whole Blood | 0.004 | 500 | 49 | GDS3919 |
| | Vaccination | | | | | |
| 128 | Obesity, Male | Cultured | 0.024 | 130 | 57 | GDS1497 |
| | | Abdominal | | | | |
| | | Subcutaneous | | | | |
| | | Mature | | | | |
| | | Adipocytes | | | | |
| 129 | Emery-Dreifuss | Muscle Biopsy | 0.008 | 500 | 58 | GDS2855 |
| | Muscular | | | | | |
| | Dystrophy | | | | | |
| 130 | Heat Stress | Skeletal | 0.04 | 500 | 65 | GDS4104 |
| | | Muscle | | | | |
| 131 | Chronic | Peripheral | 0.091 | 44 | 77 | GDS4168 |
| | Lymphocytic | Blood B Cells | | | | |
| | Leukemia | | | | | |
| 132 | Weight Loss | Skeletal | 0.024 | 500 | 69 | GDS2089 |
| | | Muscle | | | | |
| 133 | Prostate Cancer | Stromal Tissue | 0.091 | 22 | 92 | GDS4114 |
| 134 | Atopic | Non-Lesional | 0.012 | 486 | 86 | GDS2382 |
| | Dermatitis | Skin | | | | |
| 135 | H1N1 Infection | Peripheral | 0.091 | 22 | 113 | GDS4240 |
| | | Blood | | | | |
| 136 | West Nile | Retinal | 0.071 | 28 | 113 | GDS4224 |
| | Infection | Pigment | | | | |
| | | Epithelium | | | | |
| | | Cells | | | | |
| 137 | Barrett's | Endoscopic | 0.037 | 108 | 108 | GDS4350 |
| | Esophagus | Biopsy | | | | |

TABLE 15
Top 25 conserved miRNAs controlling the fibrosis network as ranked
by one-way ANOVA score. Diseases were grouped into cohorts based
on their percentage overlap with the fibrosis signature. MiRNAs
were ranked according to a one-way ANOVA means comparison test
between their assigned spanning scores in each bin. High-scoring
miRNAs were those carrying preferentially high spanning scores
in diseases with a large fibrotic component, relative to their
overall performance across diseases of all types.

| | ANOVA | ANOVA | |
|---|---|---|---|
| Rank | Score | p-value | Family |
| 1 | 25.499 | 1.2668E−12 | miR-410/344de/344b-1-3p |
| 2 | 12.140 | 1.4180E−05 | miR-144 |
| 3 | 6.109 | 0.0007 | miR-374ab |
| 4 | 4.789 | 0.0098 | miR-130ac/301ab/301b/301b- |
| | | | 3p/454/721/4295/3666 |
| 5 | 4.539 | 0.0049 | miR-155 |
| 6 | 3.790 | 0.0250 | miR-27abc/27a-3p |
| 7 | 3.508 | 0.0177 | let-7/98/4458/4500 |
| 8 | 3.411 | 0.0201 | miR-148ab-3p/152 |
| 9 | 3.366 | 0.0212 | miR-19ab |
| 10 | 3.293 | 0.0236 | miR-128/128ab |
| 11 | 3.186 | 0.0266 | miR-590-3p |
| 12 | 3.014 | 0.0331 | miR-26ab/1297/4465 |
| 13 | 2.988 | 0.0342 | miR-142-3p |
| 14 | 2.672 | 0.0509 | miR-101/101ab |
| 15 | 2.659 | 0.0518 | miR-300/381/539-3p |
| 16 | 2.208 | 0.0911 | miR-592/599 |
| 17 | 1.948 | 0.1466 | miR-29abcd |
| 18 | 1.832 | 0.1455 | miR-145 |
| 19 | 1.702 | 0.1708 | miR-17/17-5p/20ab/20b-5p/ |
| | | | 93/106ab/427/518a-3p/519d |
| 20 | 1.691 | 0.1731 | miR-22/22-3p |
| 21 | 1.678 | 0.1758 | miR-543 |
| 22 | 1.640 | 0.1843 | miR-221/222/222ab/1928 |
| 23 | 1.632 | 0.1861 | miR-199ab-5p |
| 24 | 1.285 | 0.2835 | miR-93/93a/105/106a/291a- |
| | | | 3p/294/295/302abcde/372/ |
| | | | 373/428/519a/520be/520acd- |
| | | | 3p/1378/1420ac |
| 25 | 1.28404014883 | 0.2835 | miR-205/205ab |

TABLE 16
Disease networks connected with the fibrosis network and the miR-
130/301 family share fibrotic genes with the PH network. The top
25% (n = 34/137) disease networks are listed below, as ranked
by overlap with the fibrosis network and miR-130/301 spanning score (see
also FIG. 69B). For each disease (column 4), we determined the fraction
of disease genes shared with the previously described PH network ².
In most cases, this fraction was dominated by fibrotic genes (i.e.,
shared with the fibrosis network, column 5).

| | | | Network | Fibrotic |
|---|---|---|---|---|
| | | | Fraction | Fraction of |
| | | | Shared | PH/Disease |
| Index | Disease | Tissue | with PH | Overlap |
| 1 | Helicobacter Pylori | Corpus | 0.138 | 0.891 |
| | Infection | Gastric Biopsy | | |
| 2 | Cerebral Palsy | Gracilis | 0.050 | 0.560 |
| | | Muscle | | |
| 3 | Zaire Ebolavirus | Macrophage | 0.077 | 0.714 |
| | Infection | | | |
| 4 | Alcoholic Hepatitis | Liver Tissue | 0.053 | 0.717 |
| 5 | Pterygium | Conjunctiva | 0.075 | 0.653 |
| 6 | Pituitary Gonadotrope | Tumor Tissue | 0.062 | 0.710 |
| 7 | Pituitary Null Cell | Tumor Tissue | 0.058 | 0.724 |
| | Tumor | | | |
| 8 | Helicobacter Pylori | Antumn | 0.073 | 0.466 |
| | Infection | Gastric Biopsy | | |
| 9 | Idiopathic | CD3 Cells | 0.054 | 0.815 |
| | Thrombocytopenic | | | |
| | Purpura, Remission | | | |
| 10 | Tuberous Sclerosis | Fibroblasts | 0.060 | 0.600 |
| | Complex Periungual | | | |
| | Fibroma | | | |
| 11 | Idiopathic | Whole Lung | 0.056 | 0.589 |
| | Pulmonary Fibrosis | | | |
| 12 | Acute Rotavirus | Peripheral | 0.069 | 0.623 |
| | Infection | Blood Mono- | | |
| | | nuclear Cells | | |
| 13 | Familial Combined | Peripheral | 0.071 | 0.563 |
| | Hyperlidemia | Blood | | |
| 14 | Autism Fragile | Lymphoblas- | 0.044 | 0.727 |
| | X Type | toid Cells | | |
| 15 | Schizophrenia | Postmortem | 0.064 | 0.672 |
| | | Prefrontal | | |
| | | Cortex | | |
| 16 | Tuberous | Fibroblasts | 0.072 | 0.556 |
| | Sclerosis Complex | | | |
| | Angiofibroma | | | |
| 17 | Dermatomyositis | Skeletal | 0.064 | 0.594 |
| | | Muscle Biopsy | | |
| 18 | ER Negative Breast | Tumor Tissue | 0.070 | 0.457 |
| | Cancer | | | |
| 19 | Idiopathic | Fibroblasts | 0.049 | 0.449 |
| | Pulmonary | | | |
| | Fibrosis | | | |
| 20 | Bipolar Disorder | Postmortem | 0.086 | 0.640 |
| | | Prefrontal | | |
| | | Cortex | | |
| 21 | Head and Neck | Tumor Biopsy | 0.067 | 0.597 |
| | Squamous | | | |
| | Carcinoma | | | |
| 22 | ER Positive Breast | Tumor Tissue | 0.076 | 0.526 |
| | Cancer | | | |
| 23 | UV Exposure | Skin | 0.058 | 0.621 |
| | | Fibroblasts | | |
| 24 | Morbid Obesity | Omental | 0.076 | 0.526 |
| | | Adipose | | |
| | | Tissue | | |
| 25 | Idiopathic | CD3 Cells | 0.074 | 0.459 |
| | Thrombocytopenic | | | |
| | Purpura, Active | | | |
| 26 | Uterine Fibroid | Uterine | 0.076 | 0.579 |
| | | Leiomyoma | | |
| | | Tissue | | |
| 27 | Air Pollution | Peripheral | 0.046 | 0.696 |
| | Exposure, Child | Blood | | |
| 28 | Scleroderma- | Fibroblasts | 0.048 | 0.563 |
| | Associated | | | |
| | Pulmonary Fibrosis | | | |
| 29 | Protein | Skeletal | 0.042 | 0.452 |
| | Deficiency | Muscle | | |
| 30 | Aging | Frontal Cortex | 0.068 | 0.618 |
| | | Male | | |

### References for Example 6

1. Butcher, D. T., Alliston, T. & Weaver, V. M. A tense situation: Forcing tumour progression. Nat Rev Cancer. 9, 108-122 (2009).
2. Lu, P., Weaver, V. M. & Werb, Z. The extracellular matrix: A dynamic niche in cancer progression. J Cell Biol. 196, 395-406 (2012).
3. Dupont, S., et al. Role of yap/taz in mechanotransduction. Nature. 474, 179-183 (2011).
4. Cordenonsi, M., et al. The hippo transducer taz confers cancer stem cell-related traits on breast cancer cells. Cell. 147, 759-772 (2011).
5. Harvey, K. F., Zhang, X. & Thomas, D. M. The hippo pathway and human cancer. Nat Rev Cancer. 13, 246-257 (2013).
6. Pan, D. The hippo signaling pathway in development and cancer. Dev Cell. 19, 491-505 (2010).
7. Fujii, M., et al. Tgf-beta synergizes with defects in the hippo pathway to stimulate human malignant mesothelioma growth. J Exp Med. 209, 479-494 (2012).
8. Liu, F., et al. Mechanosignaling through yap and taz drives fibroblast activation and fibrosis. Am J Physiol Lung Cell Mol Physiol. 308, L344-357 (2015).
9. Valastyan, S. & Weinberg, R. A. Roles for micrornas in the regulation of cell adhesion molecules. J Cell Sci. 124, 999-1006 (2011).
10. Parikh, V. N., et al. Microrna-21 integrates pathogenic signaling to control pulmonary hypertension: Results of a network bioinformatics approach. Circulation. 125, 1520-1532 (2012).
11. Bertero, T., et al. Systems-level regulation of microrna networks by miR-130/301 promotes pulmonary hypertension. J Clin Invest. 124, 3514-3528 (2014).
12. Pencheva, N., Buss, C. G., Posada, J., Merghoub, T. & Tavazoie, S. F. Broad-spectrum therapeutic suppression of metastatic melanoma through nuclear hormone receptor activation. Cell. 156, 986-1001 (2014).
13. Chan, S. Y. & Loscalzo, J. The emerging paradigm of network medicine in the study of human disease. Circ Res. 111, 359-374 (2012).
14. Goh, K. I., et al. The human disease network. Proc Natl Acad Sci USA. 104, 8685-8690 (2007).
15. Zhou, X., Menche, J., Barabasi, A. L. & Sharma, A. Human symptoms-disease network. Nat Commun. 5, 4212 (2014).
16. Bitanihirwe, B. K. & Woo, T. U. Perineuronal nets and schizophrenia: The importance of neuronal coatings. Neurosci Biobehav Rev. 45C, 85-99 (2014).
17. Michalik, L. & Wahli, W. Involvement of PPAR nuclear receptors in tissue injury and wound repair. J Clin Invest. 116, 598-606 (2006).
18. Lakatos, H. F., et al. The role of ppars in lung fibrosis. PPAR Res. 2007, 71323 (2007).
19. Wang, Z., et al. Peroxisome proliferator-activated receptor gamma inhibits hepatic fibrosis in rats. Hepatobiliary & pancreatic diseases international: HBPD INT. 10, 64-71 (2011).
20. Kampschulte, M., et al. Western diet in apoe-ldlr double-deficient mouse model of atherosclerosis leads to hepatic steatosis, fibrosis, and tumorigenesis. Lab Invest. 94, 1273-1282 (2014).
21. Ferre, N., et al. Increased susceptibility to exacerbated liver injury in hypercholesterolemic apoe-deficient mice: Potential involvement of oxysterols. Am J Physiol Gastrointest Liver Physiol. 296, G553-562 (2009).
22. Kothapalli, D., et al. Cardiovascular protection by apoe and apoe-hdl linked to suppression of ecm gene expression and arterial stiffening. Cell Rep. 2, 1259-1271 (2012).
23. Gressner, O. A. & Gressner, A. M. Connective tissue growth factor: A fibrogenic master switch in fibrotic liver diseases. Liver Int. 28, 1065-1079 (2008).
24. Levental, K. R., et al. Matrix crosslinking forces tumor progression by enhancing integrin signaling. Cell. 139, 891-906 (2009).
25. Barry-Hamilton, V., et al. Allosteric inhibition of lysyl oxidase-like-2 impedes the development of a pathologic microenvironment. Nat Med. 16, 1009-1017 (2010).
26. Nave, A. H., et al. Lysyl oxidases play a causal role in vascular remodeling in clinical and experimental pulmonary arterial hypertension. Arterio Thromb Vasc Biol. 34, 1446-1458 (2014).
27. Calvo, F., et al. Mechanotransduction and yap-dependent matrix remodelling is required for the generation and maintenance of cancer-associated fibroblasts. Nat Cell Biol. 15, 637-646 (2013).
28. Piccolo, S., Dupont, S. & Cordenonsi, M. The biology of yap/taz: Hippo signaling and beyond. Physiol Rev. 94, 1287-1312 (2014).
29. Plouffe, S. W., Hong, A. W. & Guan, K. L. Disease implications of the hippo/yap pathway. Trends Mol Med. 21, 212-222 (2015).
30. Liu, A. M., Xu, M. Z., Chen, J., Poon, R. T. & Luk, J. M. Targeting yap and hippo signaling pathway in liver cancer. Exp Opin Therap Targ. 14, 855-868 (2010).
31. Pottier, N., et al. Identification of keratinocyte growth factor as a target of microrna-155 in lung fibroblasts: Implication in epithelial-mesenchymal interactions. PLoS One. 4, e6718 (2009).
32. Pandit, K. V., et al. Inhibition and role of let-7d in idiopathic pulmonary fibrosis. Am J Respir Crit Care Med. 182, 220-229 (2010).
33. van Rooij, E., et al. Dysregulation of micrornas after myocardial infarction reveals a role of mir-29 in cardiac fibrosis. Proc Natl Acad Sci USA. 105, 13027-13032 (2008).
34. Xiao, J., et al. Mir-29 inhibits bleomycin-induced pulmonary fibrosis in mice. Mol Ther. 20, 1251-1260 (2012).
35. Roderburg, C., et al. Micro-ma profiling reveals a role for mir-29 in human and murine liver fibrosis. Hepatology. 53, 209-218 (2011).
36. Thum, T., et al. Microrna-21 contributes to myocardial disease by stimulating map kinase signalling in fibroblasts. Nature. 456, 980-984 (2008).
37. Liu, G., et al. MiR-21 mediates fibrogenic activation of pulmonary fibroblasts and lung fibrosis. J Exp Med. 207, 1589-1597 (2010).
38. Chau, B. N., et al. MicroRNA-21 promotes fibrosis of the kidney by silencing metabolic pathways. Sci Transl Med. 4, 121ra118 (2012).
39. da Costa Martins, P. A., et al. MicroRNA-199b targets the nuclear kinase dyrk1a in an auto-amplification loop promoting calcineurin/nfat signalling. Nat Cell Biol. 12, 1220-1227 (2010).
40. Lino Cardenas, C. L., et al. MiR-199a-5p is upregulated during fibrogenic response to tissue injury and mediates tgfbeta-induced lung fibroblast activation by targeting caveolin-1. PLoS Genet. 9, e1003291 (2013).
41. Chou, J., et al. Gata3 suppresses metastasis and modulates the tumour microenvironment by regulating microrna-29b expression. Nat Cell Biol. 15, 201-213 (2013).
42. Mouw, J. K., et al. Tissue mechanics modulate microrna-dependent pten expression to regulate malignant progression. Nat Med. 20, 360-367 (2014).
43. Tumaneng, K., et al. Yap mediates crosstalk between the hippo and pi(3)k-tor pathways by suppressing pten via miR-29. Nat Cell Biol. 14, 1322-1329 (2012).
44. Jiang, J., et al. Association of microrna expression in hepatocellular carcinomas with hepatitis infection, cirrhosis, and patient survival. Clin Cancer Res. 14, 419-427 (2008).
45. Wang, W. Y., et al. High expression of microrna-130b correlates with poor prognosis of patients with hepatocellular carcinoma. Diag Pathol. 9, 160 (2014).
46. Shi, W., et al. MicroRNA-301 mediates proliferation and invasion in human breast cancer. Cancer Res. 71, 2926-2937 (2011).
47. Ma, F., et al. Upregulated microRNA-301a in breast cancer promotes tumor metastasis by targeting pten and activating wnt/beta-catenin signaling. Gene. 535, 191-197 (2014).
48. Luo, H., Zhu, H., Zhou, B., Xiao, X. & Zuo, X. MicroRNA-130b regulates scleroderma fibrosis by targeting peroxisome proliferator-activated receptor gamma. Mod Rheum. 1-8 (2015).
49. Kim, K. M., et al. Shear stress induced by an interstitial level of slow flow increases the osteogenic differentiation of mesenchymal stem cells through taz activation. PLoS One. 9, e92427 (2014).
50. Johnson, R. & Halder, G. The two faces of hippo: Targeting the hippo pathway for regenerative medicine and cancer treatment. Nat Rev Drug Discov. 13, 63-79 (2014).
51. Bertero, T., et al. The microRNA-130/301 family controls vasoconstriction in pulmonary hypertension. J Biol Chem. 290, 2069-2085 (2014).
52. Brock, M., et al. The hypoxia-induced microRNA-130a controls pulmonary smooth muscle cell proliferation by directly targeting cdkn1a. Int J Biochem Cell Biol. 61C, 129-137 (2015).
53. Simonneau, G., et al. Updated clinical classification of pulmonary hypertension. J Am Coll Cardiol. 62,D34-41 (2013).
54. Kramann, R., et al. Perivascular gli1+progenitors are key contributors to injury-induced organ fibrosis. Cell Stem Cell. 16, 51-66 (2014).
55. Wei, C., et al. Circulating miRNAs as potential marker for pulmonary hypertension. PLoS One. 8, e64396 (2013).
56. Coussens, L. M., Fingleton, B. & Matrisian, L. M. Matrix metalloproteinase inhibitors and cancer: Trials and tribulations. Science. 295, 2387-2392 (2002).
57. Hu, J., Van den Steen, P. E., Sang, Q. X. & Opdenakker, G. Matrix metalloproteinase inhibitors as therapy for inflammatory and vascular diseases. Nat Rev Drug Discov. 6, 480-498 (2007).
58. Obad, S., et al. Silencing of microRNA families by seed-targeting tiny LNAs. Nat Genet. 43, 371-378 (2011).
59. Corey, K. E., et al. Non-high-density lipoprotein cholesterol as a biomarker for nonalcoholic steatohepatitis. Clin Gastroenterol Hepatol. 10, 651-656 (2012).
60. Corey, K. E., et al. The absence of obstructive sleep apnea may protect against non-alcoholic fatty liver in patients undergoing bariatric surgery. PLoS One. 8, e62504 (2013).
61. Parikh, V. N., et al. Microrna-21 integrates pathogenic signaling to control pulmonary hypertension: Results of a network bioinformatics approach. Circulation. 125, 1520-1532 (2012).
62. Bertero, T., et al. Systems-level regulation of microrna networks by mir-130/301 promotes pulmonary hypertension. J Clin Invest. 124, 3514-3528 (2014).
63. Blondel, V. D., Guillaume, J. L., Hendrickx, J. M., de Kerchove, C. & Lambiotte, R. Local leaders in random networks. Phys Rev E Stat Nonlin Soft Matter Phys. 77, 036114 (2008).
64. Friedman, R. C., Farh, K. K., Burge, C. B. & Bartel, D. P. Most mammalian mrnas are conserved targets of micrornas. Genome Res. 19, 92-105 (2009).
65. Barry-Hamilton, V., et al. Allosteric inhibition of lysyl oxidase-like-2 impedes the development of a pathologic microenvironment. Nat Med. 16, 1009-1017 (2010).
66. Hu, B., et al. Multifocal epithelial tumors and field cancerization from loss of mesenchymal csl signaling. Cell. 149, 1207-1220 (2012).

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. A method of inhibiting, preventing or treating pulmonary hypertension (PH) or pulmonary arterial hypertension (PAH) or a symptom thereof in a subject in need thereof, comprising inhibiting activity or expression of at least one of microRNA-130a, microRNA-130b, microRNA-301a, microRNA-301b, and microRNA-454.

2. A method for inhibiting, preventing or treating a fibrotic or fibroproliferative disease or a symptom thereof in a subject in need thereof, comprising inhibiting activity or expression of at least one of microRNA-130a, microRNA-130b, microRNA-301a, microRNA-301b, and microRNA-454.

3. (canceled)

4. A method of modulating extracellular matrix deposition or vascular/tissue stiffness in a subject in need thereof, comprising inhibiting activity or expression of at least one of microRNA-130a, microRNA-130b, microRNA-301a, microRNA-301b, and microRNA-454.

5. (canceled)

6. The method of claim 1, wherein the method comprises administering to the subject an effective amount of an inhibitor of that inhibits the activity of at least two of microRNA-130a, microRNA-130b, microRNA-301a, microRNA-301b, and microRNA-454.

7. The method of claim 1, wherein the method comprises administering to the subject an effective amount of an inhibitor of that inhibits the activity of at least three of microRNA-130a, microRNA-130b, microRNA-301a, microRNA-301b, and microRNA-454.

8. The method of claim 1, wherein the method comprises administering to the subject an effective amount of an inhibitor of that inhibits the activity of at least four of microRNA-130a, microRNA-130b, microRNA-301a, microRNA-301b, and microRNA-454.

9. The method of claim 1, wherein the method comprises inhibiting the activity of microRNA-130a, microRNA-130b, microRNA-301a, and microRNA-301b.

10. The method of claim 1, wherein the method comprises inhibiting the activity of microRNA-130a, microRNA-130b, microRNA-301a, microRNA-301b, and microRNA-454.

11. (canceled)

12. The method of claim 1, wherein the inhibitor is an oligonucleotide.

13. The method of claim 1, wherein the inhibitor is an anti-miR, antagomir, antisense oligonucleotide, ribozyme, siRNA, or shRNA.

14. The method of claim 1, wherein the inhibitor comprise a nucleotide sequence that is substantially complementary to at least a portion of nucleic acid sequence selected from the group consisting of:
(human miR-130a)
(SEQ ID NO: 20)
UGCUGCUGGCCAGAGCUCUUUUCACAUUGUGCUACUGUCUGCACCUGUCA
CUAGCAGUGCAAUGUUAAAAGGGCAUUGGCCGUGUAGUG;
(human miR-130b)
(SEQ ID NO: 21)
GGCCUGCCCGACACUCUUUCCCUGUUGCACUACUAUAGGCCGCUGGGAAG
CAGUGCAAUGAUGAAAGGGCAUCGGUCAGGUC;
(human miR-301a)
(SEQ ID NO: 22)
ACUGCUAACGAAUGCUCUGACUUUAUUGCACUACUGUACUUUACAGCUAG
CAGUGCAAUAGUAUUGUCAAAGCAUCUGAAAGCAGG;
(human miR-301b)
(SEQ ID NO: 23)
GCCGCAGGUGCUCUGACGAGGUUGCACUACUGUGCUCUGAGAAGCAGUGC
AAUGAUAUUGUCAAAGCAUCUGGGACCA;
(mouse miR-130a)
(SEQ ID NO: 24)
GAGCUCUUUUCACAUUGUGCUACUGUCUAACGUGUACCGAGCAGUGCAAU
GUUAAAAGGGCAUC;
and
(mouse miR-130b)
(SEQ ID NO: 25)
CAGUGGGCUUGUUGGACACUCUUUCCCUGUUGCACUACUGUGGGCCUCUG
GGAAGCAAUGAUGAAAGGGCAUCUGUCGGGCC.
(miR-454)
(SEQ ID NO: 27)
UCUGUUUAUCACCAGAUCCUAGAACCCUAUCAAUAUUGUCUCUGCUGUG
UAAAUAGUUCUGAGUAGUGCAAUAUUGCUUAUAGGGUUUUGGUGUUUGG
AAAGAACAAUGGGCAGG;
and
any combinations thereof.

15. The method of claim 1, wherein the inhibitor comprises a nucleotide sequence that is substantially complementary to at least a portion of nucleic acid sequence selected from the group consisting of has-miR-130a-3p (cagugcaauguuaaaagggcau) (SEQ ID NO: 4), has-miR-130b-3p (cagugcaaugaugaaagggcau) (SEQ ID NO: 5), has-miR-301a-3p (cagugcaauaguauugucaaagc) (SEQ ID NO: 5), has-miR-301b-3p (cagugcaaugauauugucaaagc) (SEQ ID NO: 7), has-miR-454-3p (uagugcaauauugcuuauagggu) (SEQ ID NO: 26), and any combinations thereof.

16. The method of claim 1, wherein the inhibitor comprises the nucleotide sequence 5′-TTGCACT-3′ (SEQ ID NO: 2) or 5′-ATTGCACT-3′ (SEQ ID NO: 3).

17.-35. (canceled)

36. The method of claim 1, further comprising selecting a subject for treatment before onset of said administering, comprising assaying a biological sample from the subject for miR-130/301 and selecting the subject who has elevated level of at least one member of miR-130/301 family.

37. The method of claim 36, wherein the miR-130/301 family member is selected from the group consisting of miR-130a, miR-130b, miR-301a, miR-301b, and any combinations thereof.

38.-40. (canceled)

41. A synthetic oligonucleotide comprising a nucleotide sequence that is substantially complementary to a at least a portion of nucleic acid sequence 5′-AGUGCAA-3′ (SEQ ID NO: 1).

42. The oligonucleotide of claim 41, wherein the oligonucleotide comprises a nucleotide sequence that is substantially complementary to at least a portion of nucleic acid sequence selected from the group consisting of cagugcaauguuaaaagggcau (hsa-miR-130a-3p), (cagugcaaugaugaaagggcau (hsa-miR-130b-3p), cagugcaauaguauugucaaagc (has-miR-301a-3p), cagugcaaugauauugucaaagc (hsa-miR-301b-3p), uagugcaauauugcuuauagggu (hsa-miR-454-3p), and any combinations thereof.

43. The oligonucleotide of claim 41, wherein the oligonucleotide comprises the nucleotide sequence 5′-TTGCACT-3′(SEQ ID NO: 2) or 5′-ATTGCACT-3′ (SEQ ID NO: 3).

44. The oligonucleotide of claim 41, wherein the oligonucleotide comprises a modification selected from the group consisting of nucleobase modifications, sugar modifications, inter-sugar linkage modifications, backbone modifications, and any combinations thereof.

45.-49. (canceled)

50. An expression vector encoding an oligonucleotide of claim 31.

51.-54. (canceled)


SEQUENCE LISTING (65 sequences)

SEQ ID NO: 1 - RNA, 7 nt, Homo sapiens
agugcaa

SEQ ID NO: 2 - DNA, 7 bp, Artificial Sequence
  Description of Artificial Sequence Synthetic oligonucleotide
ttgcact

SEQ ID NO: 3 - DNA, 8 bp, Artificial Sequence
  Description of Artificial Sequence Synthetic oligonucleotide
attgcact

SEQ ID NO: 4 - RNA, 22 nt, Homo sapiens
cagugcaauguuaaaagggcau

SEQ ID NO: 5 - RNA, 22 nt, Homo sapiens
cagugcaaugaugaaagggcau

SEQ ID NO: 6 - RNA, 23 nt, Homo sapiens
cagugcaauaguauugucaaagc

SEQ ID NO: 7 - RNA, 23 nt, Homo sapiens
cagugcaaugauauugucaaagc

SEQ ID NO: 8 - RNA, 8 nt, Unknown
  Description of Unknown 3′UTR of PPARG highly conserved putative binding site oligonucleotide
uugcacua

SEQ ID NO: 9 - RNA, 25 nt, Homo sapiens
cccuucuuccaguugcacuauucug

SEQ ID NO: 10 - RNA, 28 nt, Mus musculus
uccuucuauugauugcacuauuauuuug

SEQ ID NO: 11 - RNA, 28 nt, Rattus norvegicus
uccuucuaucgauugcacuauuauuuug

SEQ ID NO: 12 - RNA, 28 nt, Dasypus novemcinctus
uccuccucccaguugcacuauuauuuug

SEQ ID NO: 13 - RNA, 28 nt, Xenopus tropicalis
acucgcccccauuugcacuauuucuaua

SEQ ID NO: 14 - RNA, 22 nt, Homo sapiens
cagugcaauguuaaaagggcau

SEQ ID NO: 15 - RNA, 7 nt, Unknown
  Description of Unknown 3′ UTR of TIMP2 highly conserved putative binding site oligonucleotide
uugcacu

SEQ ID NO: 16 - RNA, 29 nt, Unknown
  Description of Unknown TIMP2 oligonucleotide
acccuugguagguauuagacuugcacuuu

SEQ ID NO: 17 - RNA, 29 nt, Unknown
  Description of Unknown TIMP2 oligonucleotide
gccuuucguagcauuagacuuugcacuuu

SEQ ID NO: 18 - RNA, 29 nt, Unknown
  Description of Unknown TIMP2 oligonucleotide
gccuuuuguagcauuagacuuugcacuuu

SEQ ID NO: 19 - RNA, 29 nt, Unknown
  Description of Unknown TIMP2 oligonucleotide
ucccccgguagcuguuagacuugcacuuu

SEQ ID NO: 20 - RNA, 89 nt, Homo sapiens
ugcugcuggccagagcucuuuucacauugugcuacugucugcaccugucacuagcagugcaauguuaaaagggcauuggccguguagug

SEQ ID NO: 21 - RNA, 82 nt, Homo sapiens
ggccugcccgacacucuuucccuguugcacuacuauaggccgcugggaagcagugcaaugaugaaagggcaucggucagguc

SEQ ID NO: 22 - RNA, 86 nt, Homo sapiens
acugcuaacgaaugcucugacuuuauugcacuacuguacuuuacagcuagcagugcaauaguauugucaaagcaucugaaagcagg

SEQ ID NO: 23 - RNA, 78 nt, Homo sapiens
gccgcaggugcucugacgagguugcacuacugugcucugagaagcagugcaaugauauugucaaagcaucugggacca

SEQ ID NO: 24 - RNA, 64 nt, Mus musculus
gagcucuuuucacauugugcuacugucuaacguguaccgagcagugcaauguuaaaagggcauc

SEQ ID NO: 25 - RNA, 82 nt, Mus musculus
ggcuuguuggacacucuuucccuguugcacuacugugggccucugggaagcagugcaaugaugaaagggcaucugucgggcc

SEQ ID NO: 26 - RNA, 23 nt, Homo sapiens
uagugcaauauugcuuauagggu

SEQ ID NO: 27 - RNA, 115 nt, Homo sapiens
ucuguuuaucaccagauccuagaacccuaucaauauugucucugcuguguaaauaguucugaguagugcaauauugcuuauaggguuuugguguuuggaaagaacaaugggcagg

SEQ ID NO: 28 - protein, 29 aa, Artificial Sequence
  Description of Artificial Sequence Synthetic peptide
AALEALAEALEALAEALEALAEAAAAGGC

SEQ ID NO: 29 - protein, 30 aa, Artificial Sequence
  Description of Artificial Sequence Synthetic polypeptide
AALAEALAEALAEALAEALAEALAAAAGGC

SEQ ID NO: 30 - protein, 15 aa, Artificial Sequence
  Description of Artificial Sequence Synthetic peptide
ALEALAEALEALAEA

SEQ ID NO: 31 - protein, 22 aa, Artificial Sequence
  Description of Artificial Sequence Synthetic peptide
GLFEAIEGFIENGWEGMIWDYG

SEQ ID NO: 32 - protein, 23 aa, Artificial Sequence
  Description of Artificial Sequence Synthetic peptide
GLFGAIAGFIENGWEGMIDGWYG

SEQ ID NO: 33 - protein, 48 aa, Artificial Sequence
  Description of Artificial Sequence Synthetic polypeptide
GLFEAIEGFIENGWEGMIDGWYGCGLFEAIEGFIENGWEGMIDGWYGC

SEQ ID NO: 34 - protein, 44 aa, Artificial Sequence
  Description of Artificial Sequence Synthetic polypeptide
GLFEAIEGFIENGWEGMIDGGCGLFEAIEGFIENGWEGMIDGGC

SEQ ID NO: 35 - protein, 35 aa, Artificial Sequence
  Description of Artificial Sequence Synthetic polypeptide
GLFGALAEALAEALAEHLAEALAEALEALAAGGSC

SEQ ID NO: 36 - protein, 34 aa, Artificial Sequence
  Description of Artificial Sequence Synthetic polypeptide
GLFEAIEGFIENGWEGLAEALAEALEALAAGGSC

SEQ ID NO: 37 - protein, 41 aa, Artificial Sequence
  Description of Artificial Sequence Synthetic polypeptide
GLFEAIEGFIENGWEGXIDGKGLFEAIEGFIENGWEGXIDG

SEQ ID NO: 38 - protein, 19 aa, Artificial Sequence
  Description of Artificial Sequence Synthetic peptide
LFEALLELLESLWELLLEA

SEQ ID NO: 39 - protein, 20 aa, Artificial Sequence
  Description of Artificial Sequence Synthetic peptide
GLFKALLKLLKSLWKLLLKA

SEQ ID NO: 40 - protein, 20 aa, Artificial Sequence
  Description of Artificial Sequence Synthetic peptide
GLFRALLRLLRSLWRLLLRA

SEQ ID NO: 41 - protein, 30 aa, Artificial Sequence
  Description of Artificial Sequence Synthetic polypeptide
WEAKLAKALAKALAKHLAKALAKALKACEA

SEQ ID NO: 42 - protein, 22 aa, Artificial Sequence
  Description of Artificial Sequence Synthetic peptide
GLFFEAIAEFIEGGWEGLIEGC

SEQ ID NO: 43 - protein, 26 aa, Artificial Sequence
  Description of Artificial Sequence Synthetic peptide
GIGAVLKVLTTGLPALISWIKRKRQQ

SEQ ID NO: 44 - protein, 8 aa, Artificial Sequence
  Description of Artificial Sequence Synthetic peptide
HHHHHWYG

SEQ ID NO: 45 - protein, 10 aa, Artificial Sequence
  Description of Artificial Sequence Synthetic peptide
CHKKKKKKHC

SEQ ID NO: 46 - protein, 16 aa, Artificial Sequence
  Description of Artificial Sequence Synthetic peptide
RQIKIWFQNRRMKWKK

SEQ ID NO: 47 - protein, 14 aa, Human immunodeficiency virus
GRKKRRQRRRPPQC

SEQ ID NO: 48 - protein, 27 aa, Artificial Sequence
  Description of Artificial Sequence Synthetic peptide
GALFLGWLGAAGSTMGAWSQPKKKRKV

SEQ ID NO: 49 - protein, 18 aa, Artificial Sequence
  Description of Artificial Sequence Synthetic peptide
LLIILRRRIRKQAHAHSK

SEQ ID NO: 50 - protein, 26 aa, Artificial Sequence
  Description of Artificial Sequence Synthetic peptide
GWTLNSAGYLLKINLKALAALAKKIL

SEQ ID NO: 51 - DNA, 8 bp, Artificial Sequence
  Description of Artificial Sequence Synthetic oligonucleotide
tcatacta

SEQ ID NO: 52 - DNA, 8 bp, Artificial Sequence
  Description of Artificial Sequence Synthetic oligonucleotide
ccactccc

SEQ ID NO: 53 - protein, 10 aa, Artificial Sequence
  Description of Artificial Sequence Synthetic peptide
KFFKFFKFFK

SEQ ID NO: 54 - DNA, 10 bp, Artificial Sequence
  Description of Artificial Sequence Synthetic oligonucleotide
ttctcctaat

SEQ ID NO: 55 - protein, 31 aa, Artificial Sequence
  Description of Artificial Sequence Synthetic polypeptide
SWLSKTAKKLENSAKKRISEGIAIAIQGGPR

SEQ ID NO: 56 - protein, 30 aa, Artificial Sequence
  Description of Artificial Sequence Synthetic polypeptide
ACYCRIPACIAGERRYGTCIYQGRLWAFCC

SEQ ID NO: 57 - protein, 36 aa, Artificial Sequence
  Description of Artificial Sequence Synthetic polypeptide
DHYNCVSSGGQCLYSACPIFTKIQGTCYRGKAKCCK

SEQ ID NO: 58 - protein, 42 aa, Artificial Sequence
  Description of Artificial Sequence Synthetic polypeptide
RRRPRPPYLPRPRPPPFFPPRLPPRIPPGFPPRFPPRFPGKR

SEQ ID NO: 59 - protein, 13 aa, Artificial Sequence
  Description of Artificial Sequence Synthetic peptide
ILPWKWPWWPWRR

SEQ ID NO: 60 - protein, 16 aa, Artificial Sequence
  Description of Artificial Sequence Synthetic peptide
AAVALLPAVLLALLAP

SEQ ID NO: 61 - protein, 11 aa, Artificial Sequence
  Description of Artificial Sequence Synthetic peptide
AALLPVLLAAP

SEQ ID NO: 62 - protein, 12 aa, Artificial Sequence
  Description of Artificial Sequence Synthetic peptide
RKCRIVVIRVCR

SEQ ID NO: 63 - protein, 18 aa, Artificial Sequence
  Description of Artificial Sequence Synthetic peptide
KLALKLALKALKAALKLA

SEQ ID NO: 64 - protein, 9 aa, Artificial Sequence
  Description of Artificial Sequence Synthetic peptide
RRRRRRRRR

SEQ ID NO: 65 - protein, 37 aa, Artificial Sequence
  Description of Artificial Sequence Synthetic polypeptide
LLGDFFRKSKEKIGKEFKRIVQRIKDFLRNLVPRTES